(19) United States
(12) Patent Application Publication
(10) Pub. No.: US 2024/0156961 A1
(43) Pub. Date: May 16, 2024

(54) TISSUE-SPECIFIC ANTIGENS FOR CANCER IMMUNOTHERAPY

(71) Applicant: BioNTech US Inc., Cambridge, MA (US)

(72) Inventors: Michael Steven Rooney, Boston, MA (US); Lauren Elizabeth Stopfer, Cambridge, MA (US)

(21) Appl. No.: 18/334,820

(22) Filed: Jun. 14, 2023

Related U.S. Application Data
(63) Continuation of application No. PCT/US21/62941, filed on Dec. 10, 2021.
(60) Provisional application No. 63/125,269, filed on Dec. 14, 2020.

(51) Int. Cl. A61K 39/00; A61P 35/00; A61P 37/04; C12N 5/0783

(52) U.S. Cl. CPC A61K 39/4632; A61K 39/0011; A61K 39/001193; A61K 39/4611; A61K 39/4644; A61P 35/00; A61P 37/04; C12N 5/0636; A61K 2039/53; C12N 2501/2301; C12N 2501/2307; C12N 2501/2315; C12N 2501/25; C12N 2501/26

(57) ABSTRACT

Provided herein are compositions relating to tissue-specific antigens, and methods for identifying tissue-specific antigens. Also provided herein are pharmaceutical compositions and methods of treatment that relate to tissue-specific antigens.

### CROSS REFERENCE

This application claims the benefit of U.S. Provisional Application No. 63/125,269, filed on Dec. 14, 2020, which is incorporated herein by reference in its entirety.

### SEQUENCE LISTING

The instant application contains a Sequence Listing which has been submitted electronically in XML format created on Jun. 14, 2023, is named 50401-727_601_SL.txt and is 7,722,446 bytes in size.

### BACKGROUND

Personalized immunotherapy using tumor-specific peptides has been described (Ott et al., Hematol. Oncol. Clin. N. Am. 28 (2014) 559-569). Prior to the present disclosure, cancer immunotherapies have mostly focused on epitopes thought to exhibit “tumor-specific” or “tumor-associated” expression patterns. Examples of such epitopes include MAGEA3, NY-ESO-1, and MSLN. Typically, these genes either suffer from low expression in tumors or non-negligible expression in essential normal tissues. These problems likely interfere with efficacy. However, focusing on tissue-specific antigens can change the scope of possible targets.

### SUMMARY

Provided herein are methods and compositions, including tissue-specific antigens not previously considered, such as tissue-specific antigens specific to non-essential tissues, that solve these problems. The tissue-specific epitope sequence can be expected to be presented on tumor cells or non-essential normal cells from a non-essential tissue of the same lineage and can be expected to have zero or a low expression level in essential tissues. The epitope sequence information of the tissue-specific antigens, e.g., antigens specific to a tumor from a particular tissue, can therefore be translated into therapeutic methods and compositions for diseases or conditions, e.g., cancer. In some embodiments the tissue-specific antigens are tumor antigens.

Provided herein is a composition comprising a tissue-specific antigen peptide comprising an epitope sequence of a protein encoded by a gene selected from the group consisting of ANKRD30A, COL10A1, CTCFL, PPIAL4G, POTEE, DLL3, MMP13, SSX1, DCAF4L2, MAGFA4, MAGEA11, MAGEC2, MAGEA12, PRAME, CLDN6, EPYC, KLK3, KLK2, KLK4, 1GM4, POTEG, RLN1, POTEH, SLC45A2, TSPAN10, PAGE5, CSAG1, PRDM7, TG, TSHR, RSPH6A, SCXB, HIST1H4K, ALPPL2, PRM2, PRM1, TNP1, LELP1, HMGB4, AKAP4, CETN1, UBQLN3, ACTL7A, ACTL9, ACTR12, PGK2, C2orJ33, KIF2B, ADAD1, SPATA8, CCDC70, TPD52L3, ACTL7B, DMRTB1, SYCN, CELA2A, CELA2B, PNLIPRP1, CTRC, AMY2A, SERPIN12, RBPJL, AQP12A, IAPP, KIRREL2, G6PC2, AQP12B, CYP11B1, CYP11B2, STAR, CYP11A1, and MC2R, wherein the protein is expressed by a cancer; a polynucleotide encoding the tissue-specific antigen peptide; one or more antigen presenting cells (APCs) comprising the tissue-specific antigen peptide; a T cell receptor (TCR) or an antibody, or a functional part thereof that is specific to an MHC:peptide complex, wherein the MHC:peptide complex comprises the tissue-specific antigen peptide; or a population of immune cells from a biological sample comprising at least one antigen specific T cell comprising the TCR.

In some embodiments, the tumor antigen epitope may comprise an epitope from any one of the proteins TSHR, TG, RSPH6A, SCXB, SSX1, or any combination thereof, and wherein the cancer comprises thyroid cancer.

Also provided herein is a population of T cells for cancer therapy for a human subject in need thereof, wherein the population of T cells comprises T cells that specifically recognize one of the epitope sequence of a protein encoded by a gene selected from the group consisting of ANKRD30A, COL10A1, CTCFL, PPIAL4G, POTEE, DLL3, MMP13, SSX1, DCAF4L2, MAGEA4, MAGEA11, MAGEC2, MAGEA12, PRAME, CLDN6, EPYC, KLK3, KLK2, KLK4, TGM4, POTEG, RLN1, POTEH, SLC45A2, TSPAN10, PAGE5, CSAG1, PRDM7, TG, TSHR, RSPH6A, SCXB, HIST1H4K, ALPPL2, PRM2, PRM1, TNP1, LELP1, HMGB4, AKAP4, CETN1, UBQLN3, ACTL7A, ACTL9, ACTRT2, PGK2, C2orf53. KIF2B, ADAD), SPATA8, CCDC70, TPD52L3, ACTL7B, DMRTB1, SYCN, CELA24, CELA2B, PNLIPRP1, CTRC, AMY2A, SERPINI2, RBPJL, AQP12A, IAPP, KIRREL2, G6PC2, AQP12B, CYP11B, CYP11B2, STAR, CYP11A1, and MC2R, wherein the epitope is expressed by a cancer cell of a human subject.

Provided herein is an improved ex vivo method for preparing tumor antigen-specific T cells, the method comprising: depleting CD14+ cells and/or CD25+ cells from a population of immune cells comprising antigen presenting cells (APCs) and T cells, thereby forming a CD14 and/or CD25 depleted population of immune cells comprising a first population of APCs and T cells, wherein the population of immune cells is from a biological sample from a human subject; and incubating the CD14 and/or CD25 depleted population of immune cells comprising a first population of APCs and T cells for a first time period in the presence of: FMS-like tyrosine kinase 3 receptor ligand (FLT3L), and (A) a polypeptide comprising at least one tumor antigen epitope sequence expressed by cancer cells of a human subject with cancer, or (B) a polynucleotide encoding the polypeptide; thereby forming a population of cells comprising stimulated T cells; expanding the population of cells comprising stimulated T cells, thereby forming an expanded population of cells comprising tumor antigen-specific T cells, wherein the tumor antigen-specific T cells comprise T cells that are specific to a complex comprising (i) the at least one tumor antigen epitope sequence and (ii) an MHC protein expressed by the cancer cells or APCs of the human subject of (b)(ii); and administering the expanded population of cells comprising tumor antigen-specific T cells to the human subject, wherein the tumor antigen epitope may be one or more of: ANKRD30A, COL10A1, CTCFL, PPIAL4G, POTEE, DLL3, MP13, SSX1, DCAF4L2, MAGEA4, MAGEA11, MAGEC2, MAGEA12, PRAME, CLDN6, EPYC, KLK3, KLK2, KLK4, TGM4, POTEG, RLN1, POTEH, SLC45A2, TSPAN10, PAGE5, CSAG1, PRDM7, TG, TSHR, RSPH6A, SCXB, HIST1H4K, ALPPL2, PRM2, PRM1, TNP1, LELP1, HMGB4, AKAP4, CETN1, UBQLN3, ACTL7A, ACTL9, ACMRT2, PGK2, C2orJ33, KIF2B, ADAD1, SPATA8, CCDC70, TPD52L3, ACTL7, DMRTB1, SYCN, CELA2A, CELA2B, PNLIPRP1, CTRC, AMY2A, SERPIN12, RBPJL, AQP12A, L4PP, KIRREL2, G6PC2, AQP12B, CYP11B1, CYP11B2, STAR, CYP11A1, and MC2R, wherein the epitope is expressed by a cancer cell of a human subject. In some embodiments, the tumor antigen epitope may comprise an epitope from any one of the proteins TSHR, TG, RSPH6A, SCXB, SSX1, or any combination thereof, and wherein the cancer comprises thyroid cancer.

Provided herein is a composition comprising a tissue-specific antigen peptide comprising an epitope sequence of a protein, wherein the epitope sequence has from 70% to 100% sequence identity to a peptide sequence selected from the group consisting of SEQ ID NOs 1-8962, wherein the protein is expressed by a cancer; a polynucleotide encoding the tissue-specific antigen peptide; one or more antigen presenting cells (APCs) presenting the tissue-specific antigen peptide; a T cell receptor (TCR) or an antibody, or a functional part thereof that is specific to an MHC:peptide complex, wherein the MHC:peptide complex comprises the tissue-specific antigen peptide; or a population of immune cells from a biological sample comprising at least one antigen specific T cell comprising the TCR.

Provided herein is a composition comprising: a tissue-specific antigen peptide comprising an epitope sequence of a protein, wherein the protein is expressed by a tumor of a target tissue; a polynucleotide encoding the tissue-specific antigen peptide; one or more antigen presenting cells (APCs) presenting the tissue-specific antigen peptide; a T cell receptor (TCR) or an antibody, or a functional part thereof that is specific to an MHC:peptide complex, wherein the MHC:peptide complex comprises the tissue-specific antigen peptide; or a population of immune cells from a biological sample comprising at least one antigen specific T cell comprising the TCR: wherein the epitope sequence binds to or is predicted to bind to a protein encoded by a MHC allele expressed by a human subject, and wherein the protein is encoded by a tissue-specific antigen epitope gene that has an expression level in the target tissue that is at least 2 fold more than an expression level of the tissue-specific antigen gene in each tissue of a plurality of non-target tissues that are different than the target tissue.

In some embodiments, the epitope sequence has from 70% to 100% sequence identity to a peptide sequence selected from the group consisting of SEQ ID NOs 6846 7061, 7359-7448, 7629-8099, and 8619-8744, and wherein the cancer comprises thyroid cancer.

In some embodiments, the protein comprises RBPJL, AQP12A, AQP12B, IAPP, CELA2A, CELA2B, AMY2A, CTRC, G6PC2, KIRREL2, PNLIPRP1, SERPINI2, SYNC, or any combination thereof, and wherein the cancer comprises pancreatic cancer.

In some embodiments, the epitope sequence has from 70% to 100% sequence identity to a peptide sequence selected from the group consisting of SEQ ID NOs 720-814, 989-1182, 1373-1565, 2120-2211, 2920-3009, 3101-3196, 3320-3440, 5193-5284, 6487-6579, 7062-7150, and 7539-7628, and wherein the cancer comprises pancreatic cancer.

In some embodiments, the protein comprises CYP11A1, CYP11B1, CYP11B2, MC2R, STAR, or any combination thereof, and wherein the cancer comprises adrenal cancer.

In some embodiments, the epitope sequence has from 70% to 100% sequence identity to a peptide sequence selected from the group consisting of SEQ ID NOs 2212-2523, 4817-4915, and 7449-7538, and wherein the cancer comprises adrenal cancer.

In some embodiments, the protein comprises ALPPL2, POTEE, PRAME, or any combination thereof, and wherein the cancer comprises uterine cancer.

In some embodiments, the epitope sequence has from 70% to 100% sequence identity to a peptide sequence selected from the group consisting of SEQ ID NOs 627-719, 5285-5431, and 6085-6183, and wherein the cancer comprises uterine cancer.

In some embodiments, the protein comprises KLK2, KLK3, KLK4, POTEH, POTEG, TGM4, RLN1, POTEE, PPIAL4G or any combination thereof, and wherein the cancer comprises prostate cancer.

In some embodiments, the epitope sequence has from 70% to 100% sequence identity to a peptide sequence selected from the group consisting of SEQ ID NOs 3441-4274, 5285-6084, 6580-6845, and 8100-8434, and wherein the cancer comprises prostate cancer.

In some embodiments, the protein comprises ANKRD30A, COL10A1, or a combination thereof and wherein the cancer comprises breast cancer.

In some embodiments, the epitope sequence has from 70% to 100% sequence identity to a peptide sequence selected from the group consisting of SEQ ID NOs 815-988, and 1749-1867, and wherein the cancer comprises breast cancer.

In some embodiments, the protein comprises CTCFL, PRAME, CLDN6, EPYC, or any combination thereof, and wherein the cancer comprises ovarian cancer.

In some embodiments, the epitope sequence has from 70% to 100% sequence identity to a peptide sequence selected from the group consisting of SEQ ID NOs 1659-1748, 1964-2119, 2827-2919, and 6085-6183, and wherein the cancer comprises ovarian cancer.

In some embodiments, the protein comprises CTCFL, and wherein the cancer comprises cervical cancer.

In some embodiments, the epitope sequence has from 70% to 100% sequence identity to a peptide sequence selected from the group consisting of SEQ ID NOs 1964-2119, and wherein the cancer comprises cervical cancer.

In some embodiments, the protein comprises POTEE, PPIAL4G, or a combination thereof, and wherein the cancer comprises colorectal cancer.

In some embodiments, the epitope sequence has from 70% to 100% sequence identity to a peptide sequence selected from the group consisting of SEQ ID Nos 5285-5431, and 5996-6084, and wherein the cancer comprises colorectal cancer.

In some embodiments, the protein comprises DLL3, and wherein the cancer comprises glioma.

In some embodiments, the epitope sequence has from 70% to 100% sequence identity to a peptide sequence selected from the group consisting of SEQ ID Nos 2619-2736, and wherein the cancer comprises glioma.

In some embodiments, the protein comprises MMP13, and wherein the cancer comprises head and neck cancer.

In some embodiments, the epitope sequence has from 70% to 100% sequence identity to a peptide sequence selected from the group consisting of SEQ ID Nos 4916-5010, and wherein the cancer comprises head and neck cancer.

In some embodiments, the protein comprises DCAF4L2, SSX1, or a combination thereof, and wherein the cancer comprises liver cancer.

In some embodiments, the epitope sequence has from 70% to 10(0% sequence identity to a peptide sequence selected from the group consisting of SEQ ID Nos 2524-2618, and 7359-7448, and wherein the cancer comprises liver cancer.

In some embodiments, the protein comprises SSX1, MAGEA4, PRAME, CSAG1, MAGEA12, MAGEA2, MAGEC2, PAGE5, PRDM7, SLC45A2, TSPAN10, or any combination thereof, and wherein the cancer comprises melanoma.

In some embodiments, the epitope sequence has from 70% to 100% sequence identity to a peptide sequence selected from the group consisting of SEQ ID Nos 1868-1963, 4458-4550, 4551-4637, 4638-4728, 4729-4816, 5011-5100, 6085-6183, 6184-6307, 7151-7264, 7359-7448, and 8745-8835, and wherein the cancer comprises melanoma.

In some embodiments, the protein comprises MAGEA11, MAGEA4, PRAME, or any combination thereof, and wherein the cancer comprises lung squamous cell carcinoma.

In some embodiments, the epitope sequence has from 70% to 100% sequence identity to a peptide sequence selected from the group consisting of SEQ ID Nos 4368-4457, 4638-4728, and 6085-6183, and wherein the cancer comprises lung squamous cell carcinoma.

In some embodiments, the protein comprises ACTL7A, ACTL7B, ACTL9, ACTRT2, ADAD1, AKAP4, C2orf53, CCDC70, CETN1, DMRTB1, HMGB4, KIF2B, LELP1, PGK2, PRM1, PRM2, SPATA8, TNP1, TPD52L3, UBQLN3, or any combination thereof, and wherein the cancer comprises testicular cancer.

In some embodiments, the epitope sequence has from 70% to 100% sequence identity to a peptide sequence selected from the group consisting of SEQ ID Nos 1-626, 1183-1372, 1566-1658, 2737-2826, 3010-3100, 3197-3319, 4275-4367, 5101-5192, 6308-6486, 7265-7358, 8435-8618, and 8836-8962, and wherein the cancer comprises testis cancer.

In some embodiments, the protein comprises KLK2, KLK3, KLK4, ANKRD30A, PRAME, MAGE4, or a combination thereof.

In some embodiments, the protein comprises KLK2, KLK3 or KLK4; and wherein the cancer comprises prostate cancer. In some embodiments, the epitope sequence has from 70% to 100% sequence identity to a peptide sequence selected from the group consisting of AYSEKVTEF (SEQ ID NO: 3534), GLWTGGKDTCGV (SEQ ID NO: 3468), HPEDTGQVF (SEQ ID NO: 3988), HPEYNRPLL (SEQ ID NO: 4143), QRVPVSHSF (SEQ ID NO: 3544), SESDTIRSI (SEQ ID NO: 4176), SLFHPEDTGQV (SEQ ID NO: 3775), SLQCVSLHL (SEQ ID NO: 3456), VILLGRHSL (SEQ ID NO: 3891), VLVHPQWVL (SEQ ID NO: 3757), LFHPEDTGQVF (SEQ ID NO: 3827), RPRSLQCVSL (SEQ ID NO: 3578), GYLQGLVSF (SEQ ID NO: 4094), IRNKSVILL (SEQ ID NO: 3974), KLQCVDLHV (SEQ ID NO: 3740), LLANGRMPTV (SEQ ID NO: 4029), LRPGDDSTL (SEQ ID NO: 3767), MPALPMVL (SEQ ID NO: 3874), NRPLLANDL (SEQ ID NO: 4216), SLQCVSLHL (SEQ ID NO: 3456), TWIAPPLQV (SEQ ID NO: 3784), VFQVSHSF (SEQ ID NO: 3828) and YSEKVTEFML (SEQ ID NO: 3454). In some embodiments, the epitope sequence has from 70% to 100% sequence identity to a peptide sequence selected from the group consisting of AYSEKVTEF (SEQ ID NO: 3534), HPEDTGQVF (SEQ ID NO: 3988), HPEYNRPLL (SEQ ID NO: 4143), QRVPVSHSF (SEQ ID NO: 3544), LFHPEDTGQVF (SEQ ID NO: 3827), GYLQGLVSF (SEQ ID NO: 4094), IRNKSVILL (SEQ ID NO: 3974), KLQCVDLHV (SEQ ID NO: 3740), LLANGRMPTV (SEQ ID NO: 4029), LRPGDDSTL (SEQ ID NO: 3767), MPALPMVL (SEQ ID NO: 3874), NRPLLANDL (SEQ ID NO: 4216), SLQCVSLHL (SEQ ID NO: 3456), TWIAPPLQV (SEQ ID NO: 3784), VFQVSHSF (SEQ ID NO: 3828) and YSEKVTEFML (SEQ ID NO: 3454).

In some embodiments, the protein comprises ANKRD30A; and wherein the cancer comprises breast cancer. In some embodiments, the epitope sequence has from 70% to 100% sequence identity to a peptide sequence selected from the group consisting of LLSHGAVIEV (SEQ ID NO: 831), SIPTKALEL (SEQ ID NO: 942), SQYSGQLKV (SEQ ID NO: 927), SVPNKALEL (SEQ ID NO: 941), SLSKILDTV (SEQ ID NO: 826) and SLDQKLFQL (SEQ ID NO: 827). In some embodiments, the epitope sequence has from 70% to 100% sequence identity to a peptide sequence selected from the group consisting of LLSHGAVIEV (SEQ ID NO: 831), SIPTKALEL (SEQ ID NO: 942), SVPNKALEL (SEQ ID NO: 941), SLSKILDTV (SEQ ID NO: 826) and SLDQKLFQL (SEQ ID NO: 827).

In some embodiments, the protein comprises PRAME; and wherein the cancer comprises squamous cell lung cancer: melanoma; ovarian cancer, uterine cancer, or am combination thereof. In some embodiments, the epitope sequence has from 70% to 100% sequence identity to a peptide sequence selected from the group consisting of DSLFFLRGR (SEQ ID NO: 6132), ELFSYLIEK (SEQ ID NO: 6108), FYDPEPILC (SEQ ID NO: 6166), ISISALQSL (SEQ ID NO: 6161), ITDDQLLAL (SEQ ID NO: 6158), KRKKNVLRL (SEQ ID NO: 6173), LQSLLQHLI (SEQ ID NO: 6146), LSHIHASSY (SEQ ID NO: 6152), PYLGQMINL (SEQ ID NO: 6120), QLLALLPSL (SEQ ID NO: 6093), SFYGNSISI (SEQ ID NO: 6174), SLLQHLIGL (SEQ ID NO: 6095), SPSVSQLSVL (SEQ ID NO: 6139), SPYLGQMINL (SEQ ID NO: 6138), TSPRRLVEL (SEQ ID NO: 6159), VLYPVPLESY (SEQ ID NO: 6154), VSPEPLQAL (SEQ ID NO: 6156), YLHARLREL (SEQ ID NO:6157) and RLDQLLRHV (SEQ ID NO:6104). In some embodiments, the epitope sequence has from 70% to 100% sequence identity to a peptide sequence of SLLQHLIGL (SEQ ID NO: 6095).

In some embodiments, the protein comprises MAGE4; and wherein the cancer comprises squamous cell lung cancer. In some embodiments, the epitope sequence has from 70% to 100% sequence identity to a peptide sequence selected from the group consisting of EVDPASNTY (SEQ ID NO: 4638), GVYDGREHTV (SEQ ID NO: 4653), KEVDPASNTY (SEQ ID NO: 4640), KVDELAHFL (SEQ ID NO: 4648), QIFPKTGL (SEQ ID NO: 4692), QSPQGASAL (SEQ ID NO: 4707), SALPTTISF (SEQ ID NO: 4699), TVYGEPRKL (SEQ ID NO: 4722), VYGEPRKL (SEQ ID NO: 4727), YPSLREAAL (SEQ ID NO: 4689), ALLEEEEGV (SEQ ID NO: 4698) and KVLEHVVRV (SEQ ID NO: 4697). In some embodiments, the epitope sequence has from 70% to 100% sequence identity to a peptide sequence selected from the group consisting of EVDPASNTY (SEQ ID NO: 4638), GVYDGREHTV (SEQ ID NO: 4653), KVDELAHFL (SEQ ID NO: 4648) and KVLEHVVRV (SEQ ID NO: 4697).

In some embodiments, the target tissue is a non-essential tissue.

In some embodiments, each non-target tissue is an essential tissue.

In some embodiments, tissue-specific antigen peptide is an isolated, purified, and/or synthetic peptide.

In some embodiments, the tissue-specific antigen peptide further comprises an accessory sequence flanking the epitope sequence.

In some embodiments, the polynucleotide comprises deoxyribonucleic acid (DNA).

In some embodiments, the polynucleotide comprises ribonucleic acid (RNA).

In some embodiments, the composition comprises a viral vector containing the polynucleotide.

In some embodiments, the viral vector is an adenovirus viral vector, an adeno-associated virus (AAV) viral vector, a Herpes Simplex virus (HSV) viral vector, a Semliki Forest Virus (SFV) viral vector, a lentivirus viral vector, a retrovirus viral vector, a poxvirus viral vector, an alpha virus viral vector, a vaccinia virus viral vector, a hepatitis B virus (HBV) viral vector, a human papillomavirus viral vector, or a pseudotype thereof, or any combination thereof.

In some embodiments, the tissue-specific antigen peptide activates CD8+ T cells, CD4+ T cells, or both.

Provided herein is a composition for autologous T cell therapy for a cancer in a subject in need thereof, wherein the composition comprises a population of T cells expressing an antigen specific TCR, wherein the antigen is a cancer antigen as disclosed herein. Contemplated is a population of immune cells from a biological sample comprising at least one antigen specific T cell comprising the TCR: wherein the epitope sequence binds to or is predicted to bind to a protein encoded by a MHC allele expressed by the human subject in need of the autologous T cell therapy, and the TCR binds to the epitope when presented in a complex by the protein encoded by a MHC allele expressed by the human subject, wherein the epitope is a tissue specific epitope that is encoded by a tissue-specific antigen epitope gene that has an expression level in the target tissue that is at least 2 fold more than an expression level of the tissue-specific antigen gene in each tissue of a plurality of non-target tissues that are different than the target tissue. In some embodiments, the T cell is a non-engineered cell. In some embodiments, the T cell is autologous to the subject. In some embodiments, the T cell is modified ex vivo.

In some embodiments, the TCR is specific to the tissue-specific antigen peptide in a complex with a class I MHC protein or a class II MHC protein.

In some embodiments, the at least one antigen specific T cell expresses CD8 or CD4.

In some embodiments, the at least one antigen specific T cell comprise an exogenous polynucleotide encoding the TCR.

In some embodiments, the at least one antigen specific T cell comprises a TCR that is specific for an epitope sequence that has from 70% to 100% sequence identity to a peptide sequence selected from the group consisting of SEQ ID NOs 6846-7061, 7359-7448, 7629-8099, and 8619-8744, and wherein the cancer comprises thyroid cancer.

In some embodiments, the at least one antigen specific T cell comprises a TCR that is specific for an epitope sequence from the protein RBPJL, AQP12A, AQP12B, IAPP, CELA2A, CELA2B, AMY2A, CTRC, G6PC2, KIRREL2, PNLIPRP1, SERPINI2, SYNC, or any combination thereof, and wherein the cancer comprises pancreatic cancer.

In some embodiments, the at least one antigen specific T cell comprises a TCR that is specific for an epitope sequence that has from 70% to 100% sequence identity to a peptide sequence selected from the group consisting of SEQ ID NOs 720-814, 989-1182, 1373-1565, 2120-2211, 2920-3009, 3101-3196, 3320-3440, 5193-5284, 6487-6579, 7062-7150, and 7539-7628, and wherein the cancer comprises pancreatic cancer.

In some embodiments, the at least one antigen specific T cell comprises a TCR that is specific for an epitope sequence from the protein: CYP11A1, CYP11B1, CYP11B2, MC2R, STAR, or any combination thereof, and wherein the cancer comprises adrenal cancer.

In some embodiments, the at least one antigen specific T cell comprises a TCR that is specific for an epitope sequence that has from 70% to 100% sequence identity to a peptide sequence selected from the group consisting of SEQ ID NOs 2212-2523, 4817-4915, and 7449-7538, and wherein the cancer comprises adrenal cancer.

In some embodiments, the at least one antigen specific T cell comprises a TCR that is specific for an epitope sequence from the protein: ALPPL2, POTEE, PRAME, or any combination thereof, and wherein the cancer comprises uterine cancer.

In some embodiments, the at least one antigen specific T cell comprises a TCR that is specific for an epitope sequence that has from 70% to 100% sequence identity to a peptide sequence selected from the group consisting of SEQ ID NOs 627-719, 5285-5431, and 6085-6183, and wherein the cancer comprises uterine cancer.

In some embodiments, the at least one antigen specific T cell comprises a TCR that is specific for an epitope sequence from the protein: KLK2, KLK3, KLK4, POTEH, POTEG, TGM4, RLN1, POTEE, PPIAL4G or any combination thereof, and wherein the cancer comprises prostate cancer.

In some embodiments, the at least one antigen specific T cell comprises a TCR that is specific for an epitope sequence that has from 70% to 100% sequence identity to a peptide sequence selected from the group consisting of SEQ ID NOs 3441-4274, 5285-6084, 6580-6845, and 8100-8434, and wherein the cancer comprises prostate cancer.

In some embodiments, the at least one antigen specific T cell comprises a TCR that is specific for an epitope sequence from the protein: ANKRD30A, COL10A1, or a combination thereof and wherein the cancer comprises breast cancer.

In some embodiments, the at least one antigen specific T cell comprises a TCR that is specific for an epitope sequence that has from 70% to 100% sequence identity to a peptide sequence selected from the group consisting of SEQ ID NOs 815-988, and 1749-1867, and wherein the cancer comprises breast cancer.

In some embodiments, the at least one antigen specific T cell comprises a TCR that is specific for an epitope sequence from the protein: CTCFL, PRAME, CLDN6, EPYC, or any combination thereof, and wherein the cancer comprises ovarian cancer.

In some embodiments, the at least one antigen specific T cell comprises a TCR that is specific for an epitope sequence that has from 70% to 100% sequence identity to a peptide sequence selected from the group consisting of SEQ ID NOs 1659-1748, 1964-2119, 2827-2919, and 6085-6183, and wherein the cancer comprises ovarian cancer.

In some embodiments, the at least one antigen specific T cell comprises a TCR that is specific for an epitope sequence from the protein: CTCFL, and wherein the cancer comprises cervical cancer.

In some embodiments, the at least one antigen specific T cell comprises a TCR that is specific for an epitope sequence that has from 70% to 100% sequence identity to a peptide sequence selected from the group consisting of SEQ ID NOs 1964-2119, and wherein the cancer comprises cervical cancer.

In some embodiments, the at least one antigen specific T cell comprises a TCR that is specific for an epitope sequence from the protein: POTEE, PPIAL4G, or a combination thereof, and wherein the cancer comprises colorectal cancer.

In some embodiments, the at least one antigen specific T cell comprises a TCR that is specific for an epitope sequence that has from 70% to 100% sequence identity to a peptide sequence selected from the group consisting of SEQ ID Nos 5285-5431, and 5996-6084, and wherein the cancer comprises colorectal cancer.

In some embodiments, the at least one antigen specific T cell comprises a TCR that is specific for an epitope sequence from the protein, DLL3, and wherein the cancer comprises glioma.

In some embodiments, the at least one antigen specific T cell comprises a TCR that is specific for an epitope sequence that has from 70% to 100% sequence identity to a peptide sequence selected from the group consisting of SEQ ID Nos 2619-2736, and wherein the cancer comprises glioma.

In some embodiments, the at least one antigen specific T cell comprises a TCR that is specific for an epitope sequence from the protein, MMP13, and wherein the cancer comprises head and neck cancer.

In some embodiments, the at least one antigen specific T cell comprises a TCR that is specific for an epitope sequence that has from 70% to 100% sequence identity to a peptide sequence selected from the group consisting of SEQ ID Nos 4916-5010, and wherein the cancer comprises head and neck cancer.

In some embodiments, the at least one antigen specific T cell comprises a TCR that is specific for an epitope sequence from the protein, DCAF4L2, or SSX1, or a combination thereof, and wherein the cancer comprises liver cancer.

In some embodiments, the at least one antigen specific T cell comprises a TCR that is specific for an epitope sequence that has from 70% to 100% sequence identity to a peptide sequence selected from the group consisting of SEQ ID Nos 2524-2618, and 7359-7448, and wherein the cancer comprises liver cancer.

In some embodiments, the at least one antigen specific T cell comprises a TCR that is specific for an epitope sequence from the protein: SSX1, MAGEA4, PRAME, CSAG1, MAGEA12, MAGEA2, MAGEC2, PAGE5, PRDM7, SLC45A2, TSPAN10, or any combination thereof, and wherein the cancer comprises melanoma.

In some embodiments, the at least one antigen specific T cell comprises a TCR that is specific for an epitope sequence that has from 70% to 100% sequence identity to a peptide sequence selected from the group consisting of SEQ ID Nos 1868-1963, 4458-4550, 4551-4637, 4638-4728, 4729-4816, 5011-5100, 6085-6183, 6184-6307, 7151-7264, 7359-7448, and 8745-8835, and wherein the cancer comprises melanoma.

In some embodiments, the at least one antigen specific T cell comprises a TCR that is specific for an epitope sequence from the protein: MAGEA11, MAGEA4, PRAME, or any combination thereof, and wherein the cancer comprises lung squamous cell carcinoma.

In some embodiments, the at least one antigen specific T cell comprises a TCR that is specific for an epitope sequence that has from 70% to 100% sequence identity to a peptide sequence selected from the group consisting of SEQ ID Nos 4368-4457, 4638-4728, and 6085-6183, and wherein the cancer comprises lung squamous cell carcinoma.

In some embodiments, the at least one antigen specific T cell comprises a TCR that is specific for an epitope sequence from the protein: ACTL7A, ACTL7B, ACTL9, ACTRT2, ADAD1, AKAP4, C2orf53, CCDC70, CETN1, DMRTB1, HMGB4, KIF2B, LELP1, PGK2, PRM1, PRM2, SPATA8, TNP1, TPD52L3, UBQLN3, or any combination thereof, and wherein the cancer comprises testicular cancer.

In some embodiments, the at least one antigen specific T cell comprises a TCR that is specific for an epitope sequence that has from 70% to 100% sequence identity to a peptide sequence selected from the group consisting of SEQ ID Nos 1-626, 1183-1372, 1566-1658, 2737-2826, 3010-3100, 3197-3319, 4275-4367, 5101-5192, 6308-6486, 7265-7358, 8435-8618, and 8836-8962, and wherein the cancer comprises testis cancer.

In some embodiments, the composition comprises the at least one antigen specific T cell, and wherein the tissue-specific antigen peptide comprises an epitope sequence of a protein encoded by a gene selected from the group consisting of: ANKRD30A, DLL3, PRAME, CLDN6, EPYC, SLC45A2, TSPAN10, TSHR, LELP1, AQP12A, KIRREL2, G6PC2, AQP12B, and MC2R.

In some embodiments, the biological sample is from a subject with the cancer or a donor other than a subject with the cancer.

In some embodiments, the donor has a natural immune response to the tissue-specific antigen peptide.

In some embodiments, the cancer comprises prostate cancer, and wherein the donor is female.

In some embodiments, the cancer comprises breast cancer or ovarian cancer, and wherein the donor is male.

In some embodiments, the protein is encoded by a tissue-specific antigen epitope gene that has an mRNA expression level in each non-target tissue of a plurality of non-target tissues that are different than a target tissue of the tumor that is at most about 5 mRNA transcripts per one million total mRNA transcripts in each respective non-target tissue.

In some embodiments, the protein is encoded by a tissue-specific antigen epitope gene that has an mRNA expression level in a target tissue that is at least about 100 mRNA transcripts per one million total mRNA transcripts in the target tissue.

Provided herein is a pharmaceutical composition comprising a composition described herein, and a pharmaceutically acceptable carrier.

Provided herein is a method comprising identifying an epitope sequence, wherein the epitope sequence binds to or is predicted to bind to a protein encoded by a MHC allele expressed by a human subject, and is encoded by a tissue-specific antigen epitope gene that has an expression level in a tumor from a target tissue that is at least 2 fold greater than an expression level of the tissue-specific antigen epitope gene in each tissue of a plurality of non-target tissues that are different than the target tissue.

Provided herein is a method of preparing T cells comprising a T cell receptor (TCR) specific to a complex of (i) a epitope sequence of a tissue specific antigen peptide of a protein and (ii) a protein encoded by an HLA allele of a human subject, the method comprising: incubating T cells in the presence of antigen presenting cells (APCs) comprising the epitope sequence, wherein the APCs express the protein encoded by an HLA allele of a human subject.

In some embodiments, the APCs comprise a polypeptide comprising the epitope sequence or a polynucleotide encoding a polypeptide comprising the epitope sequence. In some embodiments, the APCs are APCs from a human subject. In some embodiments, the T cells are T cells from a human subject. In some embodiments, the method further comprises administering the T cells to a human subject in need thereof.

Provided herein is a method of treatment, comprising: administering a composition to a human subject in need thereof, wherein the composition comprises: a tissue-specific antigen peptide comprising an epitope sequence of a protein, wherein the epitope sequence is expressed by the tumor; a polynucleotide encoding the tissue-specific antigen peptide; one or more antigen presenting cells (APCs) presenting the tissue-specific antigen peptide; a T cell receptor (TCR) specific to the tissue-specific antigen peptide; or a population of immune cells from a biological sample comprising at least one antigen specific T cell comprising the TCR, wherein the epitope sequence binds to or is predicted to bind to a protein encoded by a MHC allele expressed by the human subject, and wherein the protein is encoded by a tissue-specific antigen epitope gene that has an expression level in the tumor that is at least 2 fold more than an expression level of the tissue-specific antigen gene in each tissue of a plurality of non-target tissues that are different than the target tissue.

In some embodiments, each tissue of the plurality of tissues is an essential tissue.

In some embodiments, the plurality of tissues comprise skeletal muscle, coronary artery, heart, adipose, uterus, vagina, skin, salivary gland, brain, lung, esophagus, stomach, colon, small intestine, nerve, or any combination thereof.

In some embodiments, each non-target tissue of the plurality of non-target tissues is a non-essential tissue.

In some embodiments, the MHC allele is a class I MHC allele or a class II MHC allele.

Provided herein is a method of treating a cancer, comprising: administering a composition described herein to a subject in need thereof.

In some embodiments, the cancer comprises adrenal gland cancer, breast cancer, cervical cancer, colorectal cancer, fallopian tube cancer, glioma, head and neck cancer, liver cancer, squamous cell lung cancer, melanoma, ovarian cancer, pancreatic cancer, prostate cancer, testicular cancer, thyroid cancer, uterine cancer, or any combination thereof.

In some embodiments, the protein comprises KLK2, KLK3, KLK4, ANKRD30A, PRAME, MAGE4, or a combination thereof. In some embodiments, the protein comprises KLK2, KLK3 or KLK4; and wherein the cancer comprises prostate cancer. In some embodiments, the epitope sequence is AYSEKVTEF (SEQ ID NO: 3534) and the human subject expresses a protein encoded by an HLA-C06:02 or HLA-A24:02 allele, the epitope sequence is GLWTGGKDTCGV (SEQ ID NO: 3468) and the human subject expresses a protein encoded by an HLA-A02:01 allele, the epitope sequence is HPEDTGQVF (SEQ ID NO: 3988) and the human subject expresses a protein encoded by an HLA-C*04:01 or HLA-C07:01 allele, the epitope sequence is HPEYNRPLL (SEQ ID NO: 4143) and the human subject expresses a protein encoded by an HLA-C*07:01 or HLA-B07:02 allele, the epitope sequence is QRVPVSHSF (SEQ ID NO: 3544) and the human subject expresses a protein encoded by an HLA-C*07:01, HLA-C*07:02 or HLA-A24:02 allele, the epitope sequence is SESDTIRSI (SEQ ID NO: 4176) and the human subject expresses a protein encoded by an HLA-B13:02 allele, the epitope sequence is SLFHPEDTGQV (SEQ ID NO: 3775) and the human subject expresses a protein encoded by an HLA-A02:01 allele, the epitope sequence is SLQCVSLHL (SEQ ID NO: 3456) and the human subject expresses a protein encoded by an HLA-A02:01 allele, the epitope sequence is VILLGRHSL (SEQ ID NO: 3891) and the human subject expresses a protein encoded by an HLA-B08:01 allele, the epitope sequence is VLVHPQWVL (SEQ ID NO: 3757) and the human subject expresses a protein encoded by an HLA-A02:01 allele, the epitope sequence is LFHPEDTGQVF (SEQ ID NO: 3827) and the human subject expresses a protein encoded by an HLA-A24:02 allele, the epitope sequence is RPRSLQCVSL (SEQ ID NO: 3578) and the human subject expresses a protein encoded by an HLA-B07:02 allele, the epitope sequence is GYLQGLVSF (SEQ ID NO: 4094) and the human subject expresses a protein encoded by an HLA-A24:02 allele, the epitope sequence is IRNKSVILL (SEQ ID NO: 3974) and the human subject expresses a protein encoded by an HLA-C*06:02, HLA-C*07:02 or HLA-C07:01 allele, the epitope sequence is KLQCVDLHV (SEQ ID NO: 3740) and the human subject expresses a protein encoded by an HLA-A02:01 allele, the epitope sequence is LLANGRMPTV (SEQ ID NO: 4029) and the human subject expresses a protein encoded by an HLA-A02:01 allele, the epitope sequence is LRPGDDSTL (SEQ ID NO: 3767) and the human subject expresses a protein encoded by an HLA-C07:02 allele, the epitope sequence is MPALPMVL (SEQ ID NO: 3874) and the human subject expresses a protein encoded by an HLA-B07:02 allele, the epitope sequence is NRPLLANDL (SEQ ID NO: 4216) and the human subject expresses a protein encoded by an HLA-C*06:02, HLA-C*07:02 or HLA-C01:02 allele, the epitope sequence is SLQCVSLHL (SEQ ID NO: 3456) and the human subject expresses a protein encoded by an HLA-A02:01 allele, the epitope sequence is TWIAPPLQV (SEQ ID NO: 3784) and the human subject expresses a protein encoded by an HLA-C*04:01 or HLA-A02:01 allele, the epitope sequence is VFQVSHSF (SEQ ID NO: 3828) and the human subject expresses a protein encoded by an HLA-C*07:02 or HLA-A24:02 allele, or the epitope sequence is YSEKVTEFML (SEQ ID NO: 3454) and the human subject expresses a protein encoded by an HLA-A01:01 allele.

In some embodiments, the protein comprises ANKRD30A; and wherein the cancer comprises breast cancer. In some embodiments, the epitope sequence is LLSHGAVIEV (SEQ ID NO: 831) and the human subject expresses a protein encoded by an HLA-A02:01 allele, the epitope sequence is SQYSGQLKV (SEQ ID NO: 927) and the human subject expresses a protein encoded by an HLA-B13:02 allele, the epitope sequence is SVPNKALEL (SEQ ID NO: 941) and the human subject expresses a protein encoded by an HLA-C*04:01 or HLA-C01:02 allele, the epitope sequence is SLSKILDTV (SEQ ID NO: 826) and the human subject expresses a protein encoded by an HLA-A02:01 allele, the epitope sequence is SIPTKALEL (SEQ ID NO: 942) and the human subject expresses a protein encoded by an HLA-C*04:01 or HLA-C01:02 allele, or the epitope sequence is SLDQKLFQL (SEQ ID NO: 827) and the human subject expresses a protein encoded by an HLA-A02:01 allele.

In some embodiments, the protein comprises PRAME; and wherein the cancer comprises squamous cell lung cancer; melanoma: ovarian cancer, uterine cancer, or any combination thereof. In some embodiments, the epitope sequence is DSLFFLRGR (SEQ ID NO: 6132) and the human subject expresses a protein encoded by an HLA-A33:03 allele, the epitope sequence is ELFSYLIEK (SEQ ID NO: 6108) and the human subject expresses a protein encoded by an HLA-A03:01 allele, the epitope sequence is FYDPEPILC (SEQ ID NO: 6166) and the human subject expresses a protein encoded by an HLA-C04:01 allele, the epitope sequence is ISISALQSL (SEQ ID NO: 6161) and the human subject expresses a protein encoded by an HLA-C03:04 allele, the epitope sequence is ITDDQLLAL (SEQ ID NO: 6158) and the human subject expresses a protein encoded by an HLA-A01:01 allele, the epitope sequence is KRKKNVLRL (SEQ ID NO: 6173) and the human subject expresses a protein encoded by an HLA-C07:01 allele, the epitope sequence is LQSLLQHLI (SEQ ID NO: 6146) and the human subject expresses a protein encoded by an HLA-B13:02 allele, the epitope sequence is LSHIHASSY (SEQ ID NO: 6152) and the human subject expresses a protein encoded by an HLA-B46:01 allele, the epitope sequence is PYLGQMINL (SEQ ID NO: 6120) and the human subject expresses a protein encoded by an HLA-A24:02 allele, the epitope sequence is QLLALLPSL (SEQ ID NO: 6093) and the human subject expresses a protein encoded by an HLA-A02:01 allele, the epitope sequence is SFYGNSISI (SEQ ID NO: 6174) and the human subject expresses a protein encoded by an HLA-C07:01 allele, the epitope sequence is SLLQHLIGL (SEQ ID NO: 6095) and the human subject expresses a protein encoded by an HLA-A02:01 allele, the epitope sequence is SPSVSQLSVL (SEQ ID NO: 6139) and the human subject expresses a protein encoded by an HLA-B07:02 allele, the epitope sequence is SPYLGQMINL (SEQ ID NO: 6138) and the human subject expresses a protein encoded by an HLA-B07:02 allele, the epitope sequence is TSPRRLVEL (SEQ ID NO: 6159) and the human subject expresses a protein encoded by an HLA-C01:02 allele, the epitope sequence is VLYPVPLESY (SEQ ID NO: 6154) and the human subject expresses a protein encoded by an HLA-A03:01 allele, the epitope sequence is VSPEPLQAL (SEQ ID NO: 6156) and the human subject expresses a protein encoded by an HLA-C01:02 allele, the epitope sequence is YLHARLREL (SEQ ID NO:6157) and the human subject expresses a protein encoded by an HLA-B08:01 allele, or the epitope sequence is RLDQLLRHV (SEQ ID NO:6104) and the human subject expresses a protein encoded by an HLA-A02:01 allele.

In some embodiments, the protein comprises MAGE4: and wherein the cancer comprises squamous cell lung cancer. In some embodiments, the epitope sequence is EVDPASNTY (SEQ ID NO: 4638) and the human subject expresses a protein encoded by an HLA-A01:01 allele, the epitope sequence is GVYDGREHTV (SEQ ID NO: 4653) and the human subject expresses a protein encoded by an HLA-A02:01 allele, the epitope sequence is KEVDPASNTY (SEQ ID NO: 4640) and the human subject expresses a protein encoded by an HLA-A01:01 allele, the epitope sequence is KVDELAHFL (SEQ ID NO: 4648) and the human subject expresses a protein encoded by an HLA-A02:01 allele, the epitope sequence is QIFPKTGL (SEQ ID NO: 4692) and the human subject expresses a protein encoded by an HLA-B08:01 allele, the epitope sequence is QSPQGASAL (SEQ ID NO: 4707) and the human subject expresses a protein encoded by an HLA-C01:02 allele, the epitope sequence is SALPTTISF (SEQ ID NO: 4699) and the human subject expresses a protein encoded by an HLA-B46:01 allele, the epitope sequence is TVYGEPRKL (SEQ ID NO: 4722) and the human subject expresses a protein encoded by an HLA-C07:01 allele, the epitope sequence is VYGEPRKL (SEQ ID NO: 4727) and the human subject expresses a protein encoded by an HLA-C07:02 allele, the epitope sequence is YPSLREAAL (SEQ ID NO: 4689) and the human subject expresses a protein encoded by an HLA-B07:02 allele, the epitope sequence is ALLEEEEGV (SEQ ID NO: 4698) and the human subject expresses a protein encoded by an HLA-A02:01 allele, or the epitope sequence is KVLEHVVRV (SEQ ID NO: 4697) and the human subject expresses a protein encoded by an HLA-A02:01 allele.

Provided herein is a method comprising (a) contacting a T cell with an antigen peptide in complex with an HLA of an APC; and (b) determining a sequence of a TCR of the T cell that recognizes the antigen peptide in complex with the HLA, wherein the T cell is suspected to have zero or reduced immune tolerance to a tissue of origin of the antigen peptide. In some embodiments, the T cell is from a female subject, and the antigen peptide is specific to a tissue selected from the group consisting of: Bulbourethral gland, epididymis, penis, prostate, scrotum, seminal vesicle, testicle. In some embodiments, the T cell is from a female subject, and the antigen peptide is specific to prostate. In some embodiments, the T cell is from a male subject, and the antigen peptide is specific to a tissue selected from the group consisting of: Bartholin's gland, fallopian tube, ovary, Skene's gland, uterus, cervix, vagina, and any combination thereof. In some embodiments, the T cell is from a male subject, and the antigen peptide is specific to ovary. In some embodiments, the T cell is from a Type I diabetes patient, and the antigen peptide is specific to pancreas. In some embodiments, the T cell is from a subject that has auto-immune thyroid condition, and the antigen peptide is specific to thyroid. In some embodiments, the T cell is from a subject that is negative for an allele of the HLA. The In some embodiments, the T cell is from a subject that is negative for an allele of the HLA and the antigen peptide binds to the HLA encoded by the allele of the HLA

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 is a boxplot illustrating expression levels of gene ANKRD30A in a number of different normal tissues and tumors.

FIG. 2 is a boxplot illustrating expression levels of gene COL10A1 in a number of different normal tissues and tumors.

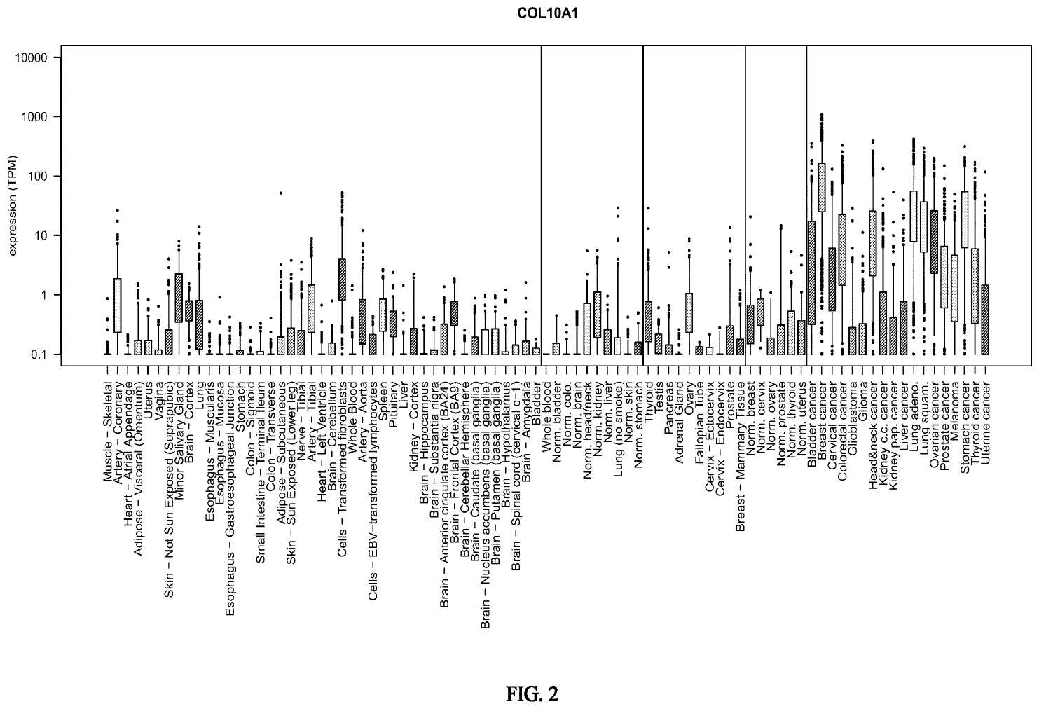

FIG. 3 is a boxplot illustrating expression levels of gene CTCFL in a number of different normal tissues and tumors.

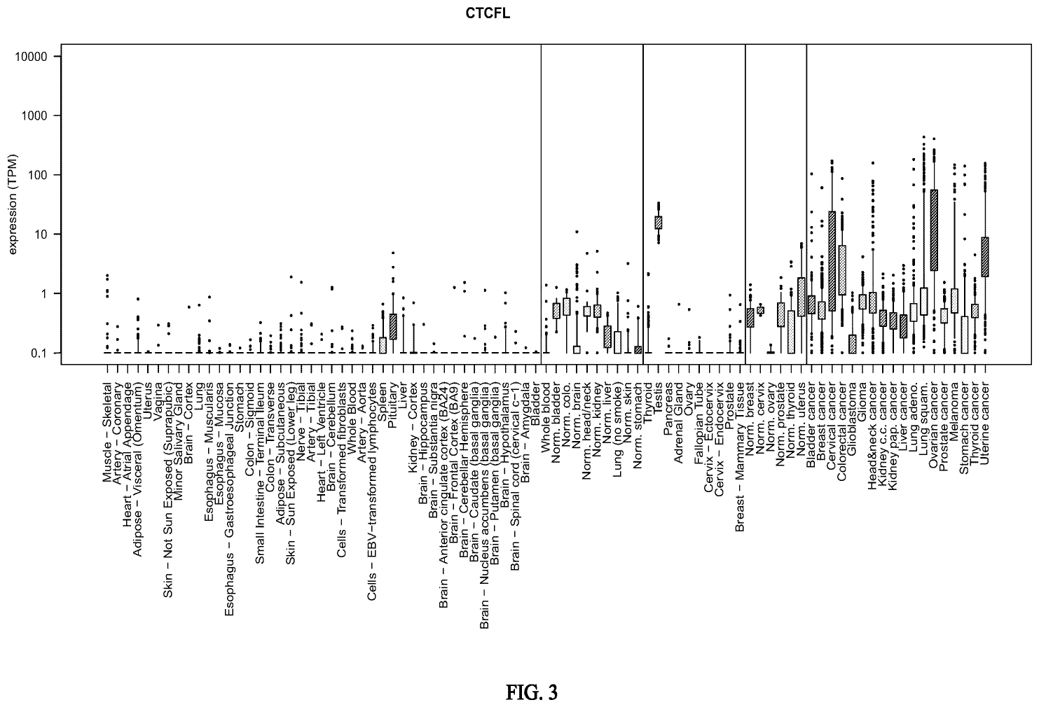

FIG. 4 is a boxplot illustrating expression levels of gene PPIAL4G in a number of different normal tissues and tumors.

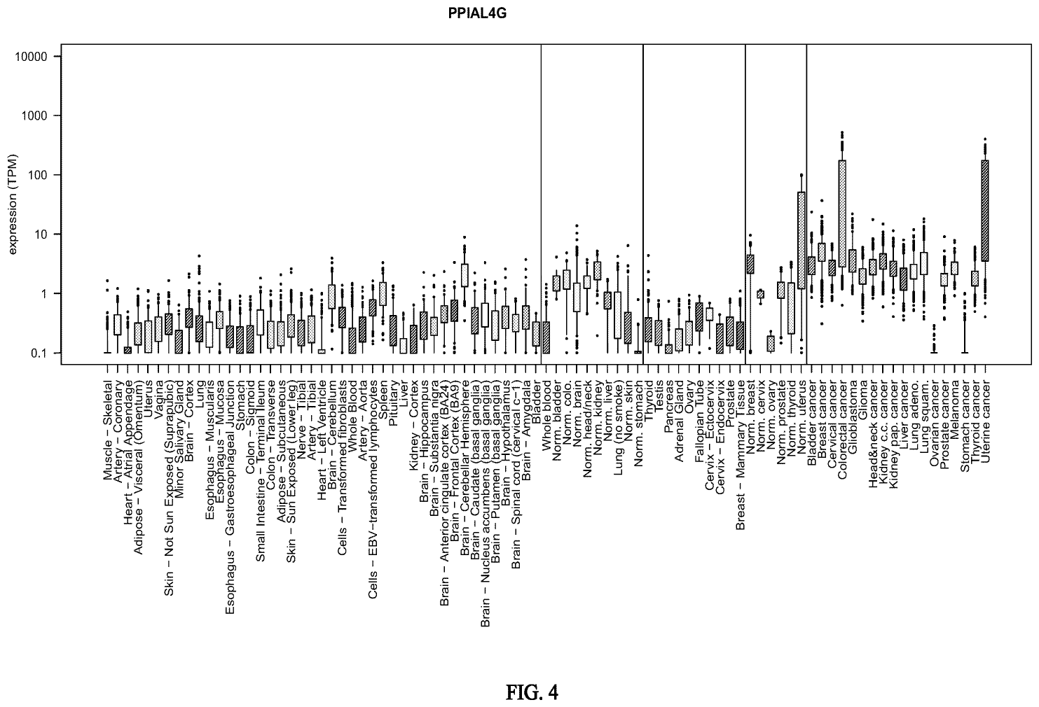

FIG. 5 is a boxplot illustrating expression levels of gene POTEE in a number of different normal tissues and tumors.

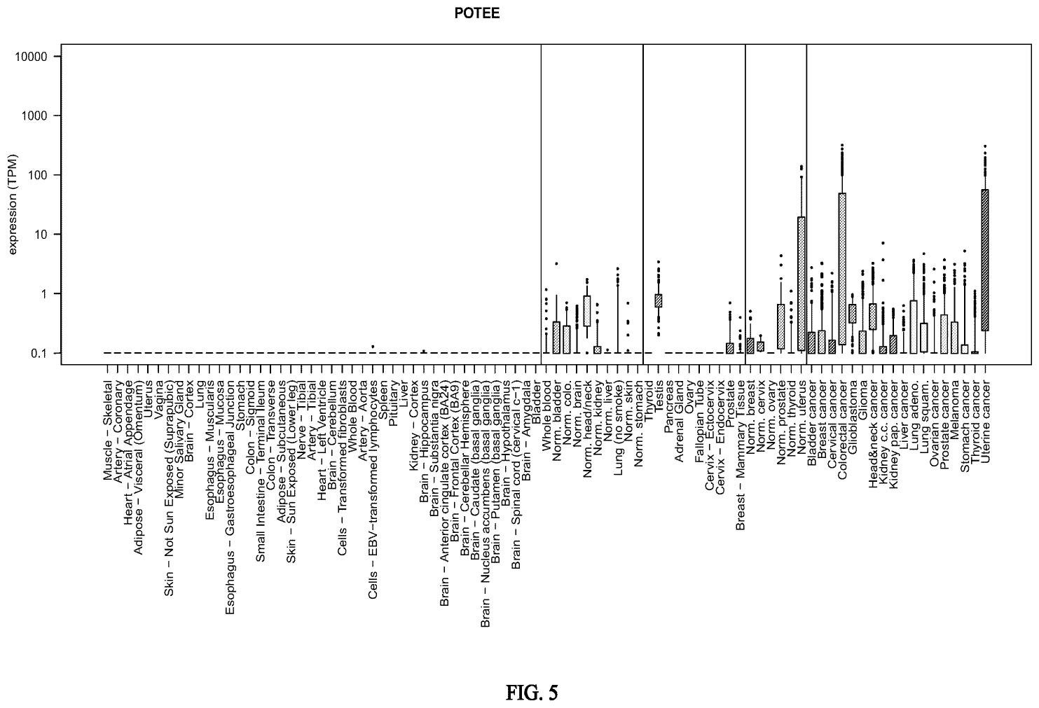

FIG. 6 is a boxplot illustrating expression levels of gene DLL3 in a number of different normal tissues and tumors.

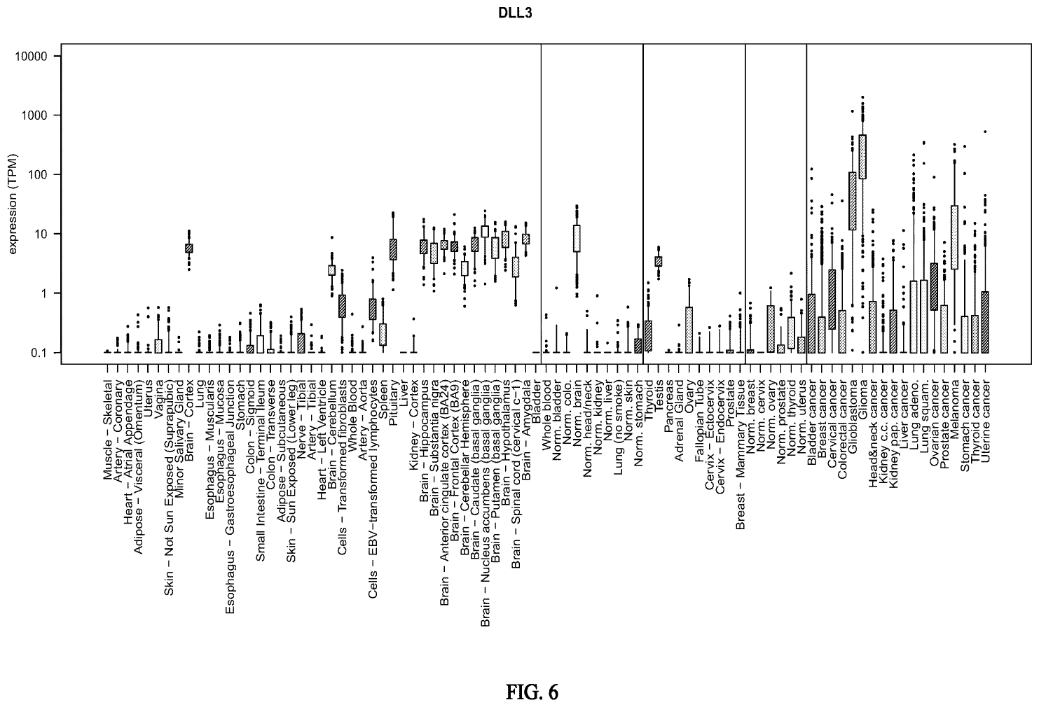

FIG. 7 is a boxplot illustrating expression levels of gene MMP13 in a number of different normal tissues and tumors.

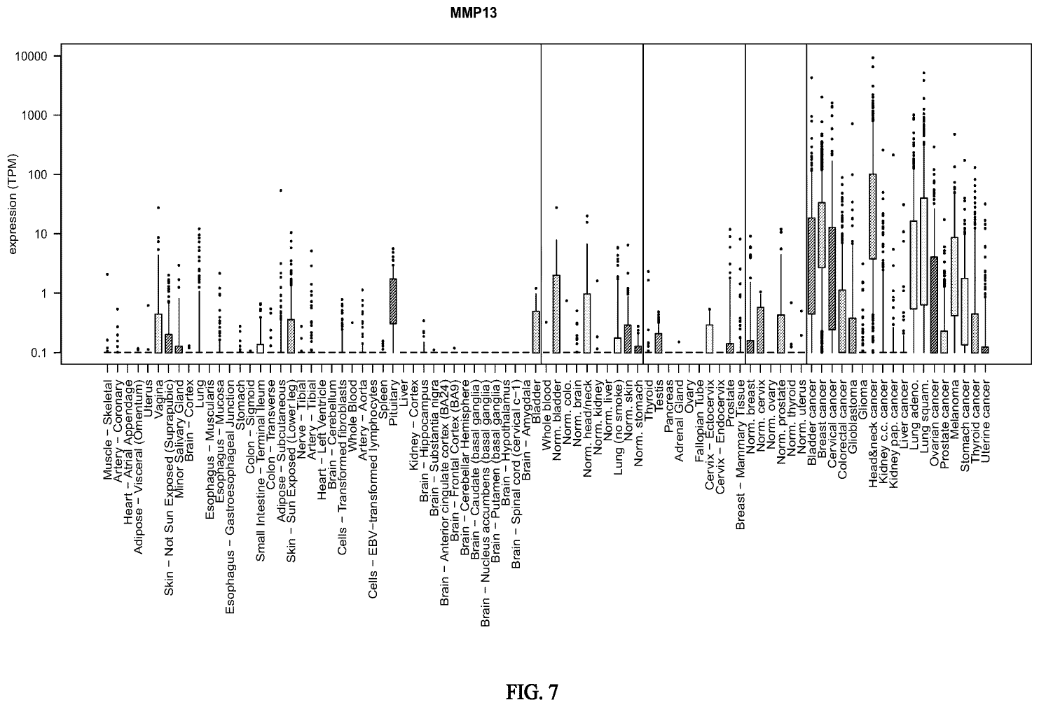

FIG. 8 is a boxplot illustrating expression levels of gene SSX1 in a number of different normal tissues and tumors.

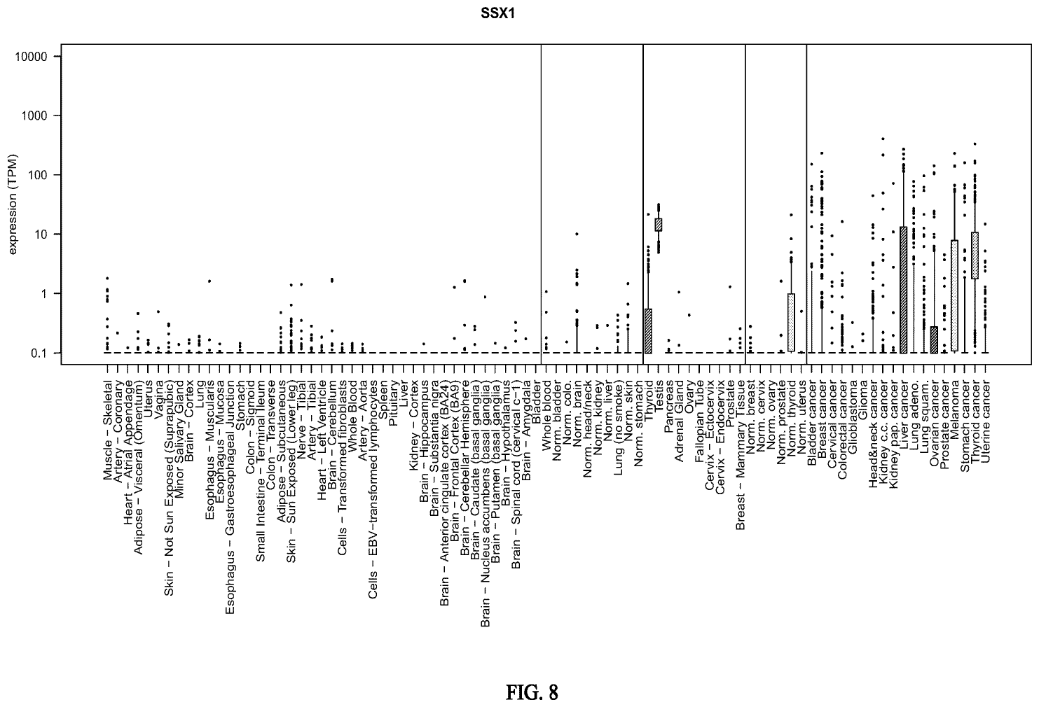

FIG. 9 is a boxplot illustrating expression levels of gene DCAF4L2 in a number of different normal tissues and tumors.

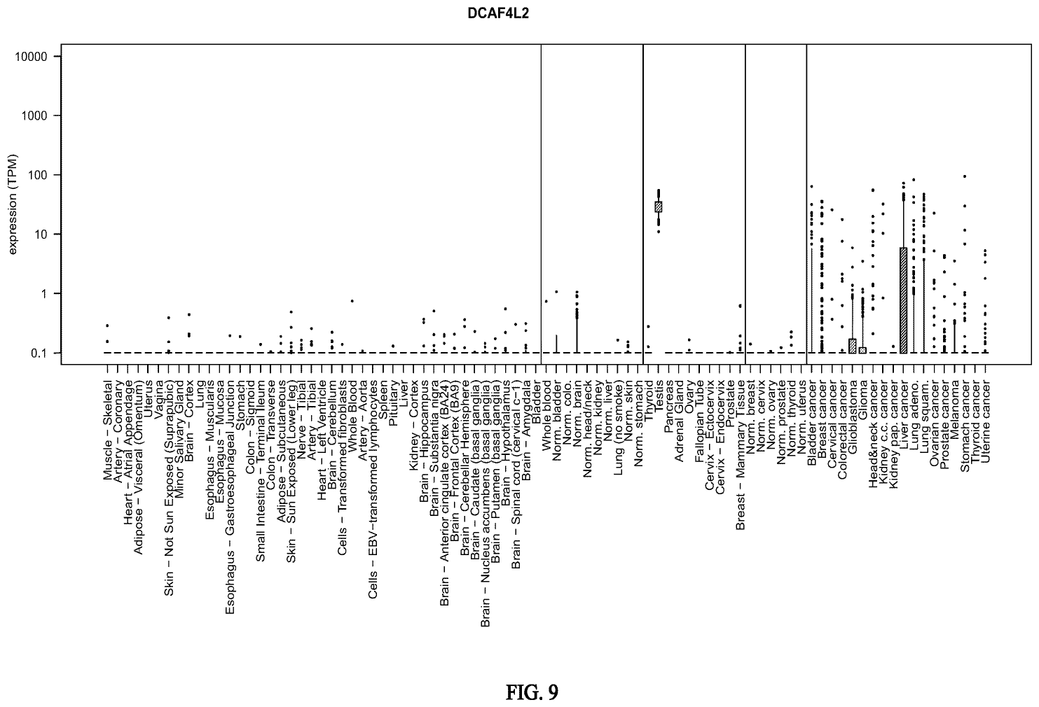

FIG. 10 is a boxplot illustrating expression levels of gene MAGEA4 in a number of different normal tissues and tumors.

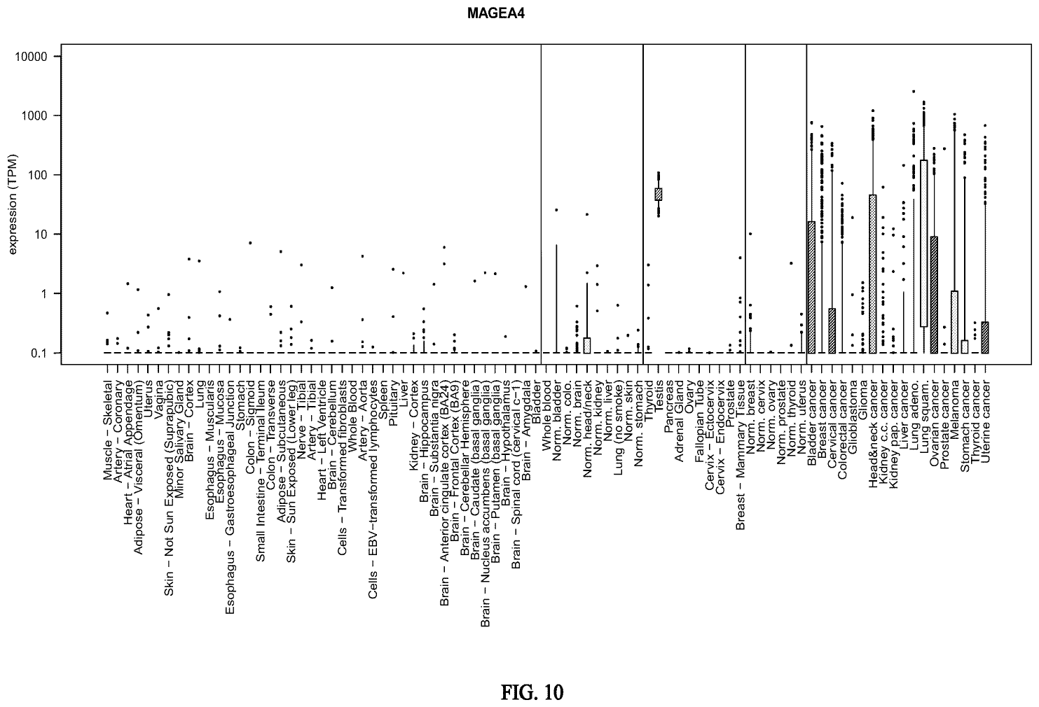

FIG. 11 is a boxplot illustrating expression levels of gene MAGEA11 in a number of different normal tissues and tumors.

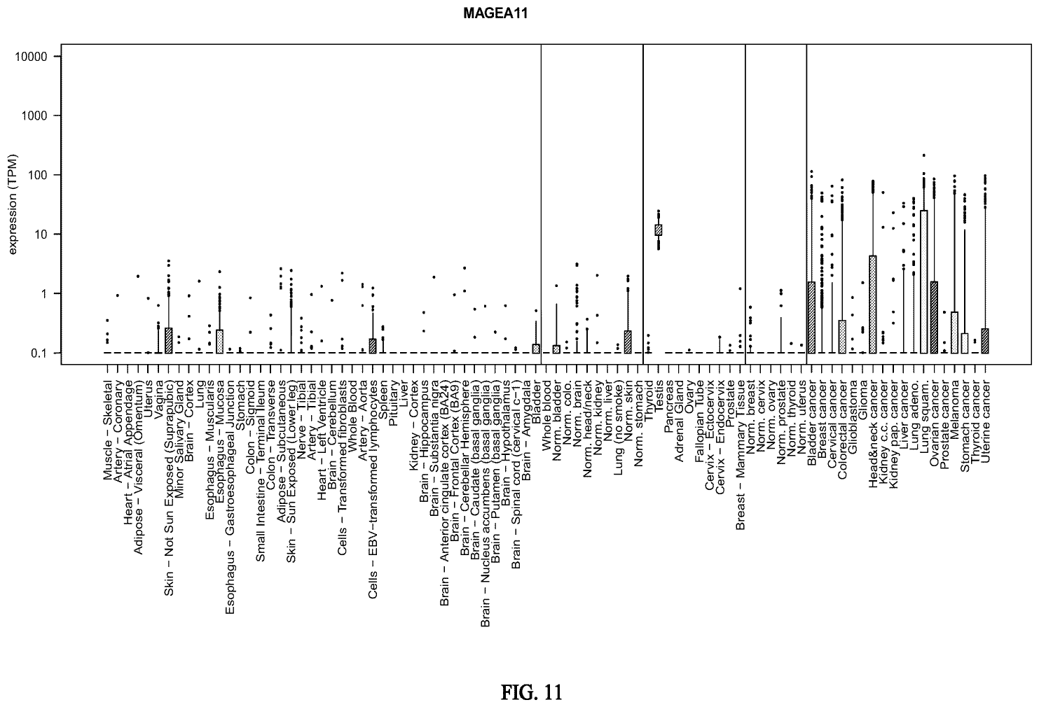

FIG. 12 is a boxplot illustrating expression levels of gene MAGEC2 in a number of different normal tissues and tumors.

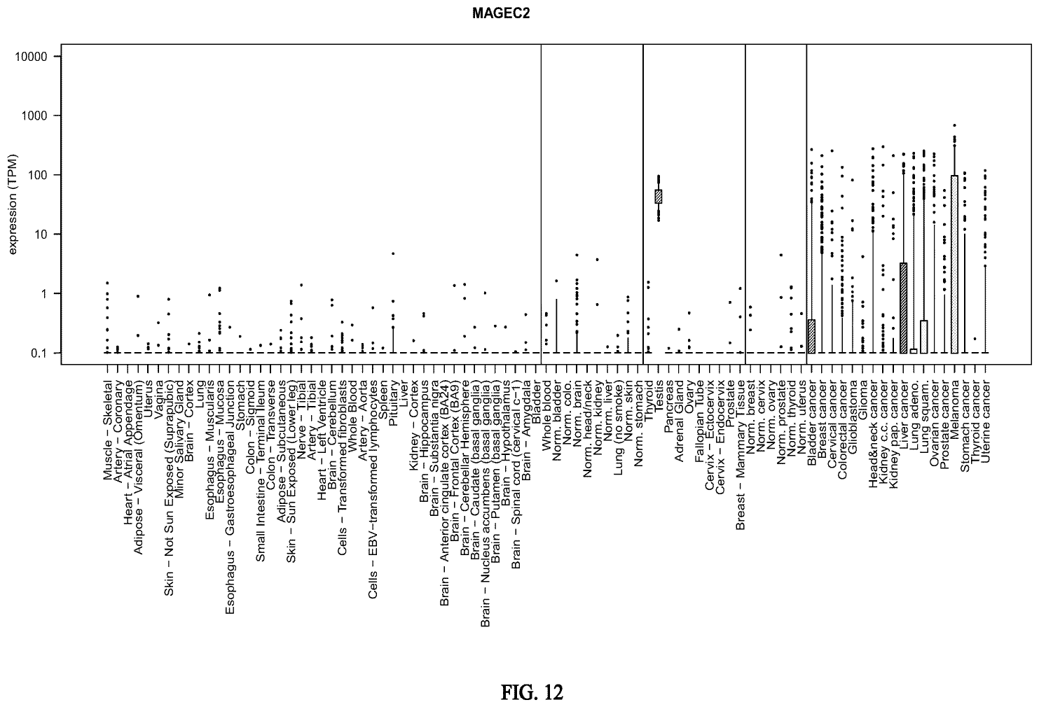

FIG. 13 is a boxplot illustrating expression levels of gene MAGEA12 in a number of different normal tissues and tumors.

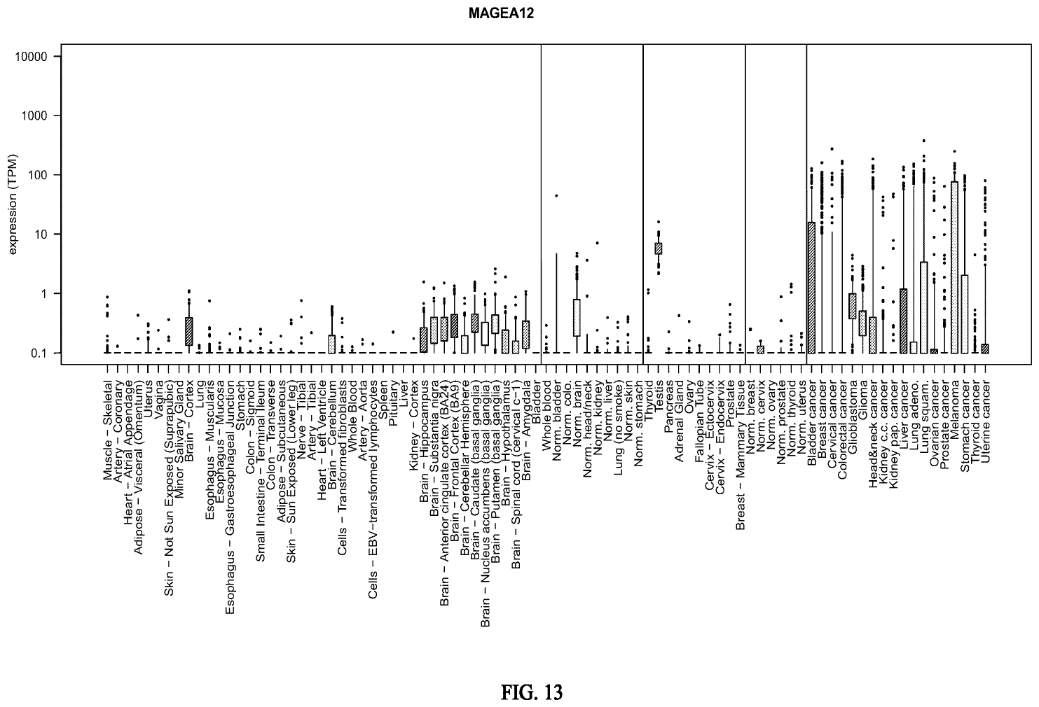

FIG. 14 is a boxplot illustrating expression levels of gene PRAME in a number of different normal tissues and tumors.

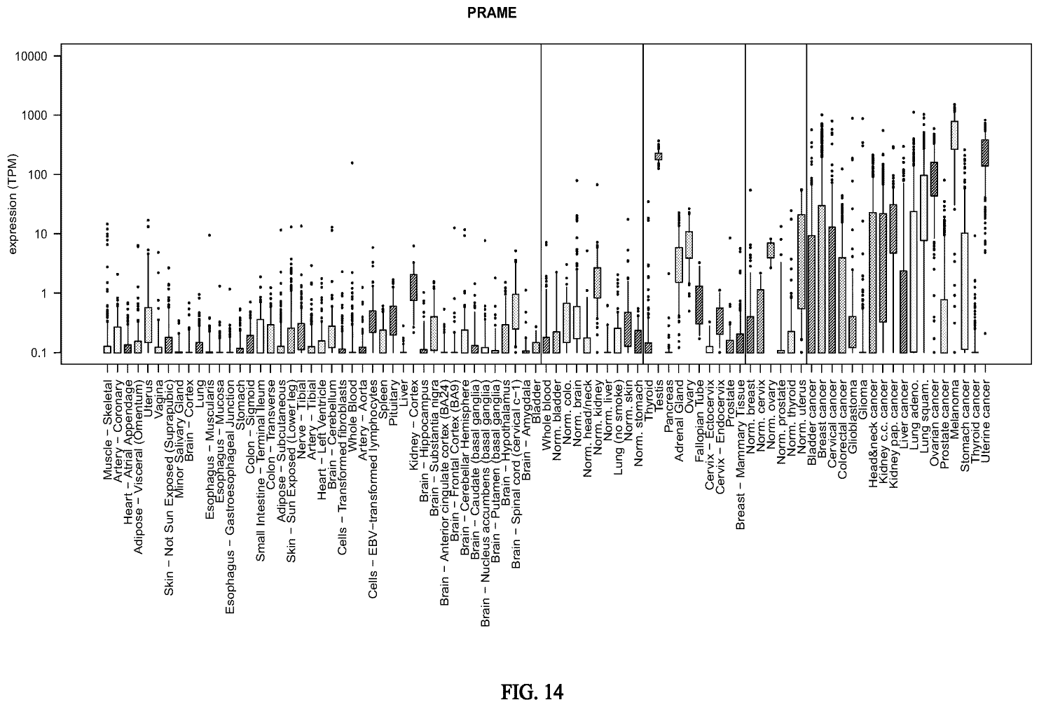

FIG. 15 is a boxplot illustrating expression levels of gene CLDN6 in a number of different normal tissues and tumors.

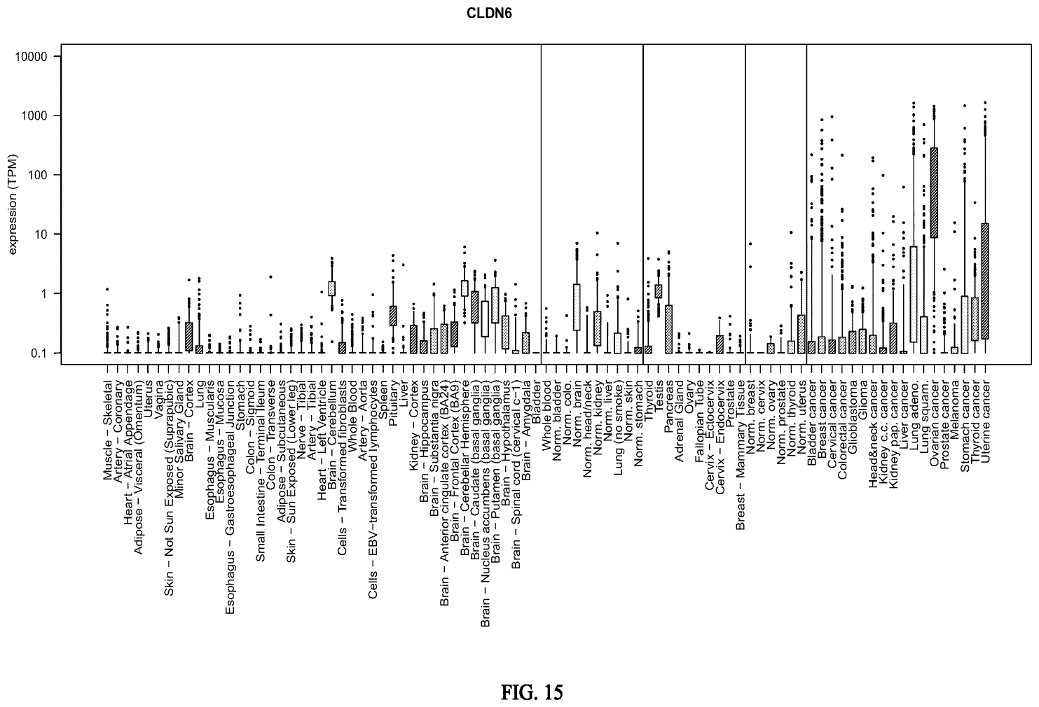

FIG. 16 is a boxplot illustrating expression levels of gene EPYC in a number of different normal tissues and tumors.

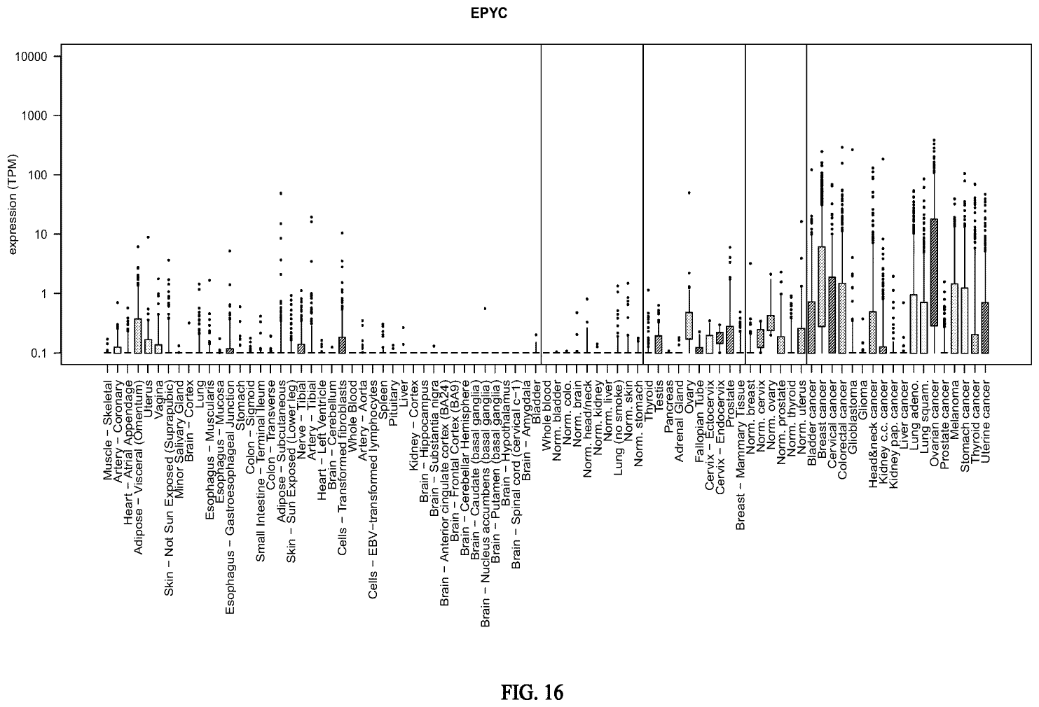

FIG. 17 is a boxplot illustrating expression levels of gene KLK3 in a number of different normal tissues and tumors.

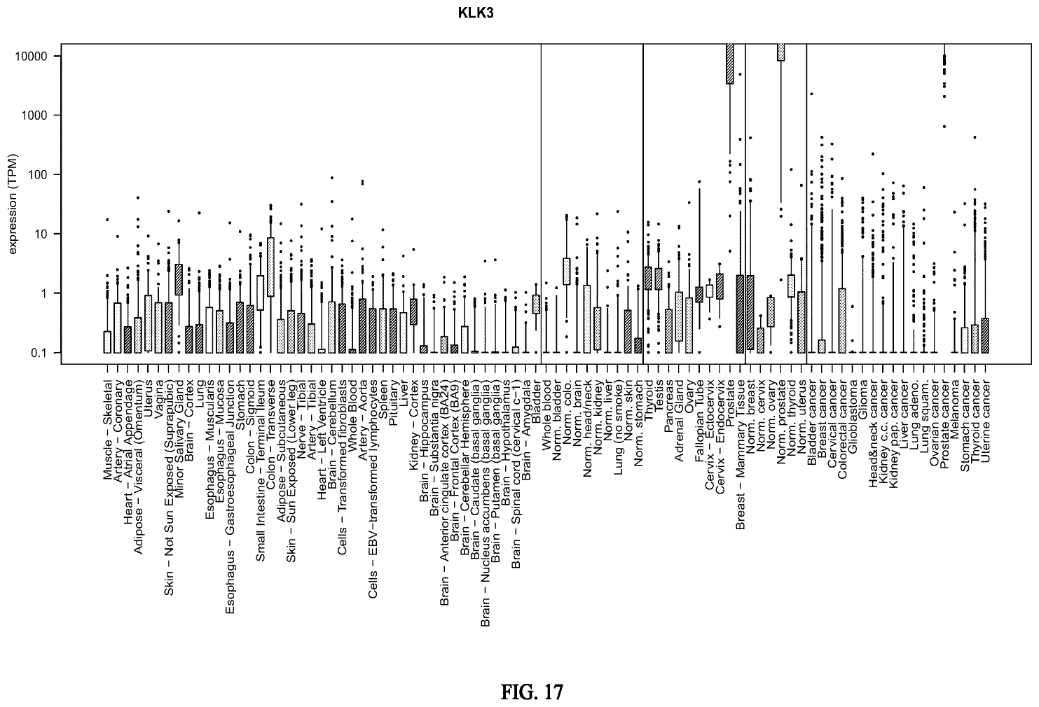

FIG. 18 is a boxplot illustrating expression levels of gene KLK2 in a number of different normal tissues and tumors.

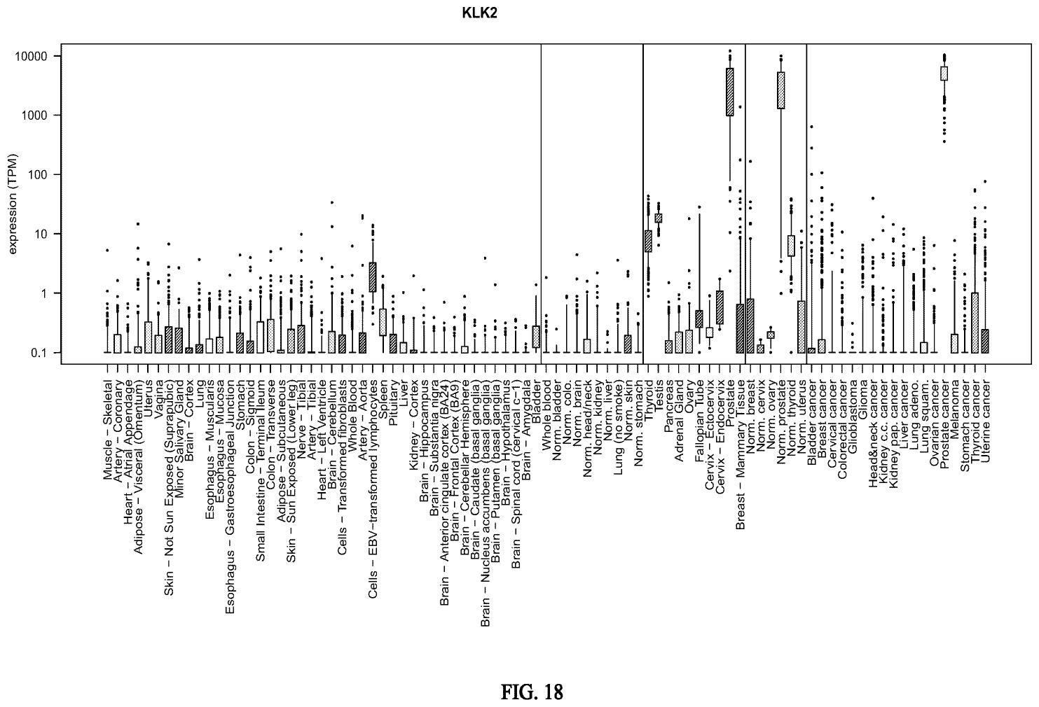

FIG. 19 is a boxplot illustrating expression levels of gene KLK4 in a number of different normal tissues and tumors.

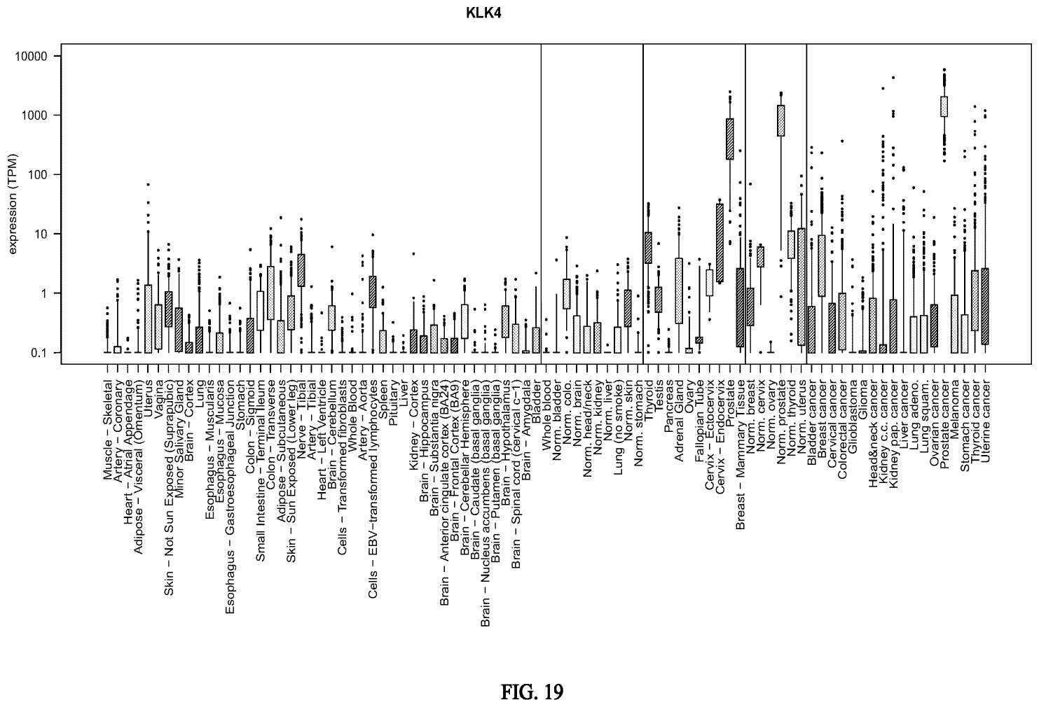

FIG. 20 is a boxplot illustrating expression levels of gene TGM4 in a number of different normal tissues and tumors.

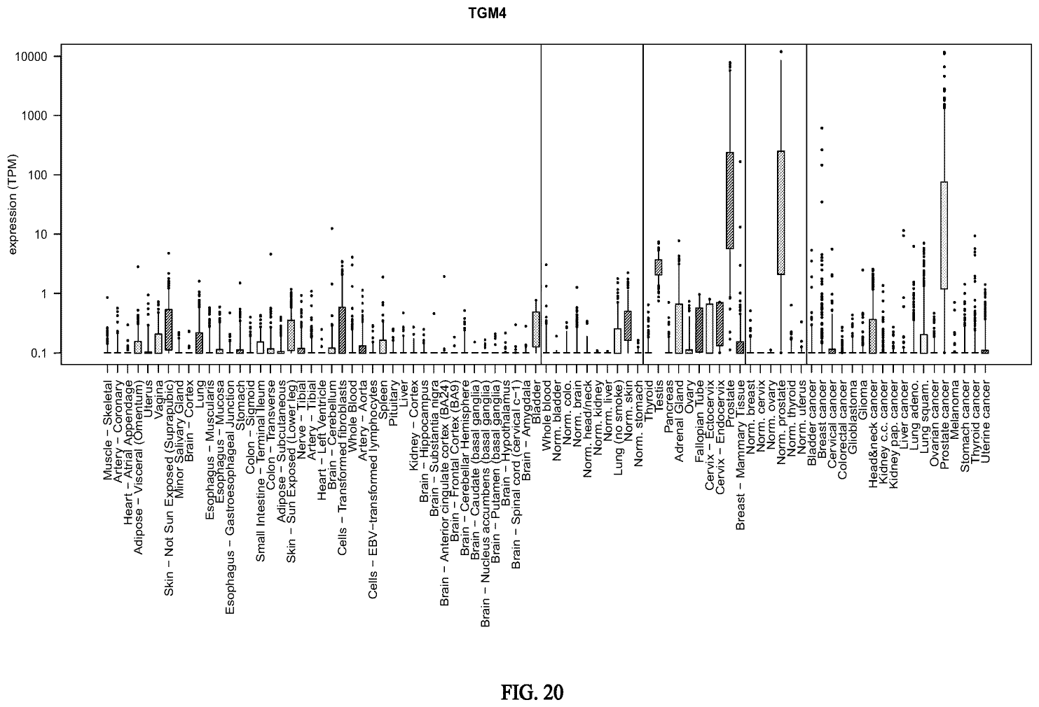

FIG. 21 is a boxplot illustrating expression levels of gene POTEG in a number of different normal tissues and tumors.

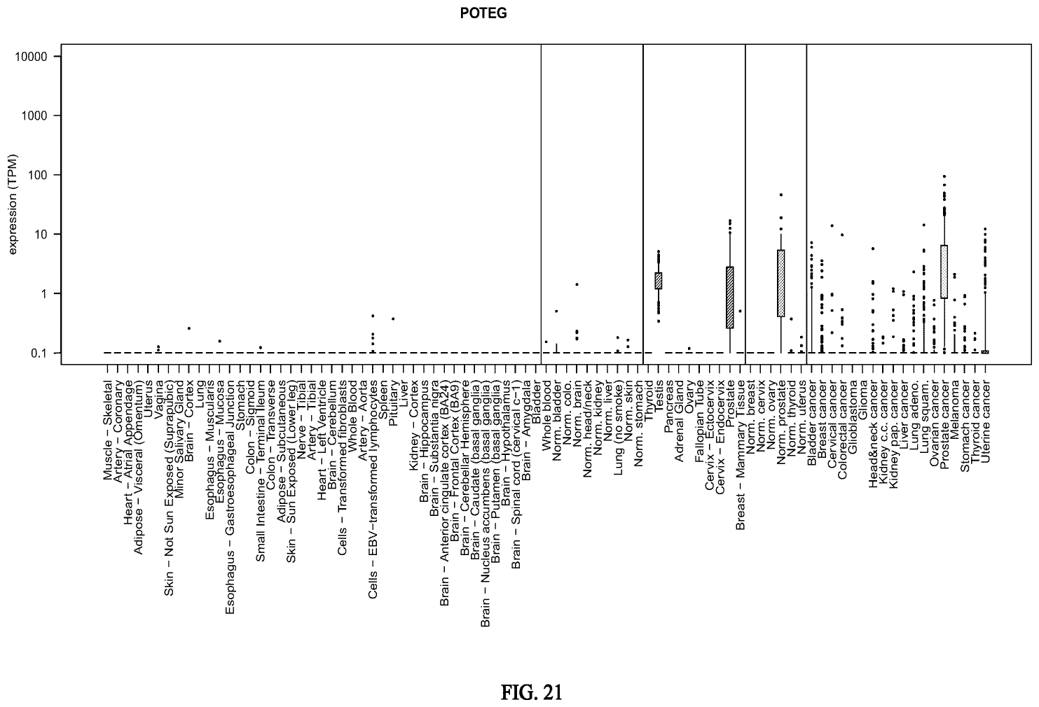

FIG. 22 is a boxplot illustrating expression levels of gene RLN1 in a number of different normal tissues and tumors.

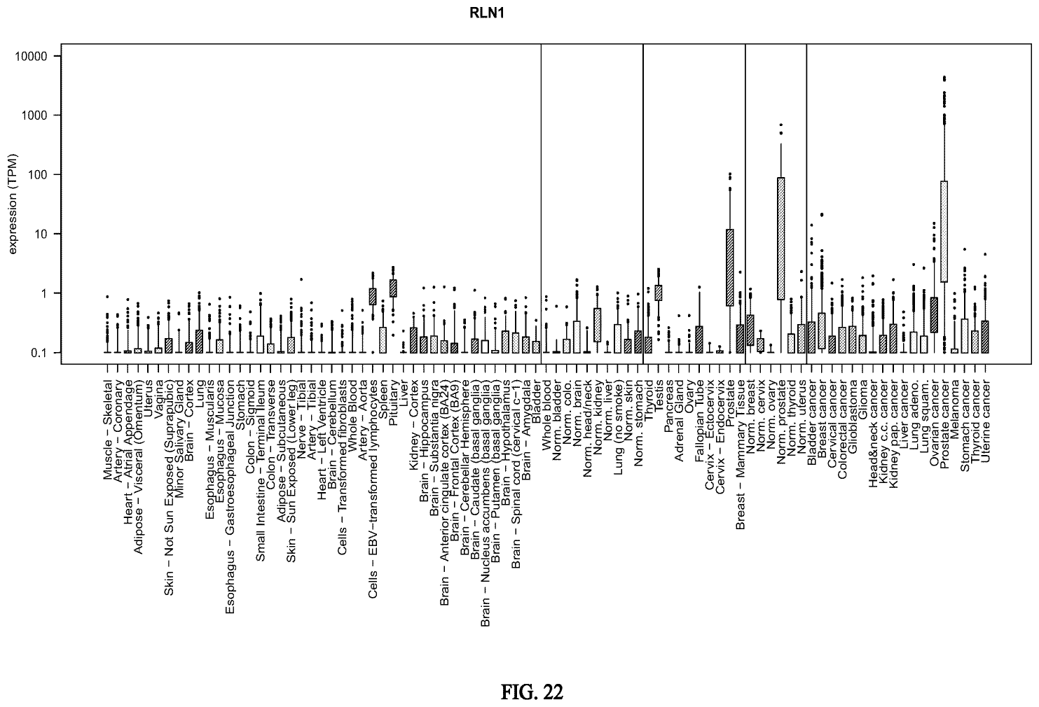

FIG. 23 is a boxplot illustrating expression levels of gene POTEH in a number of different normal tissues and tumors.

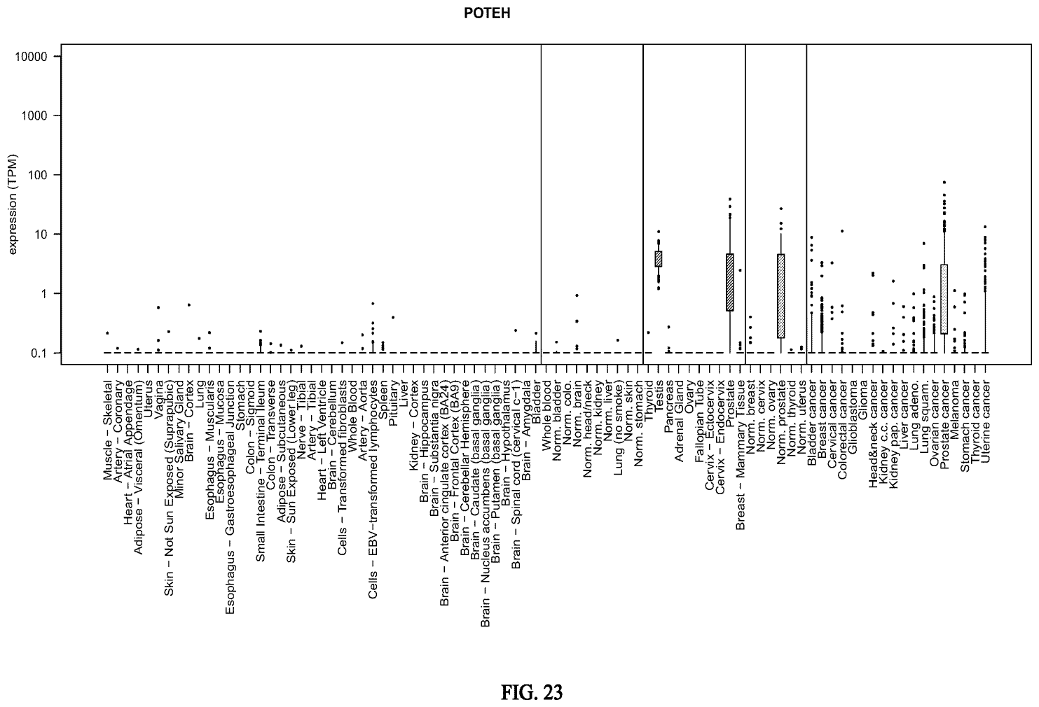

FIG. 24 is a boxplot illustrating expression levels of gene SLC45A2 in a number of different normal tissues and tumors.

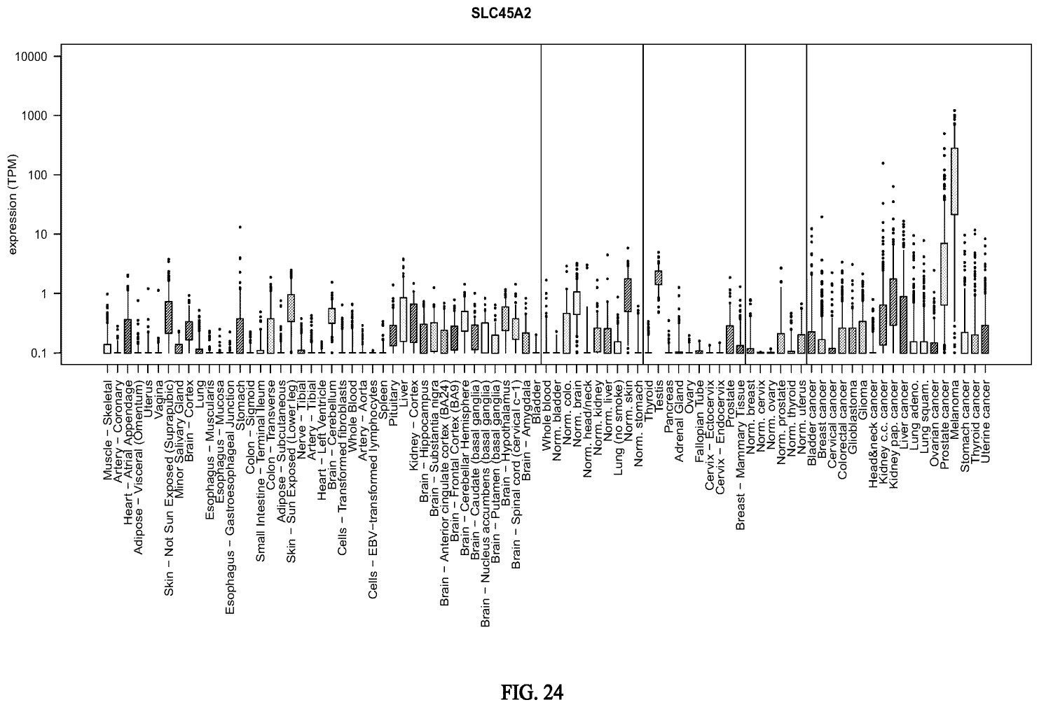

FIG. 25 is a boxplot illustrating expression levels of gene TSPAN10 in a number of different normal tissues and tumors.

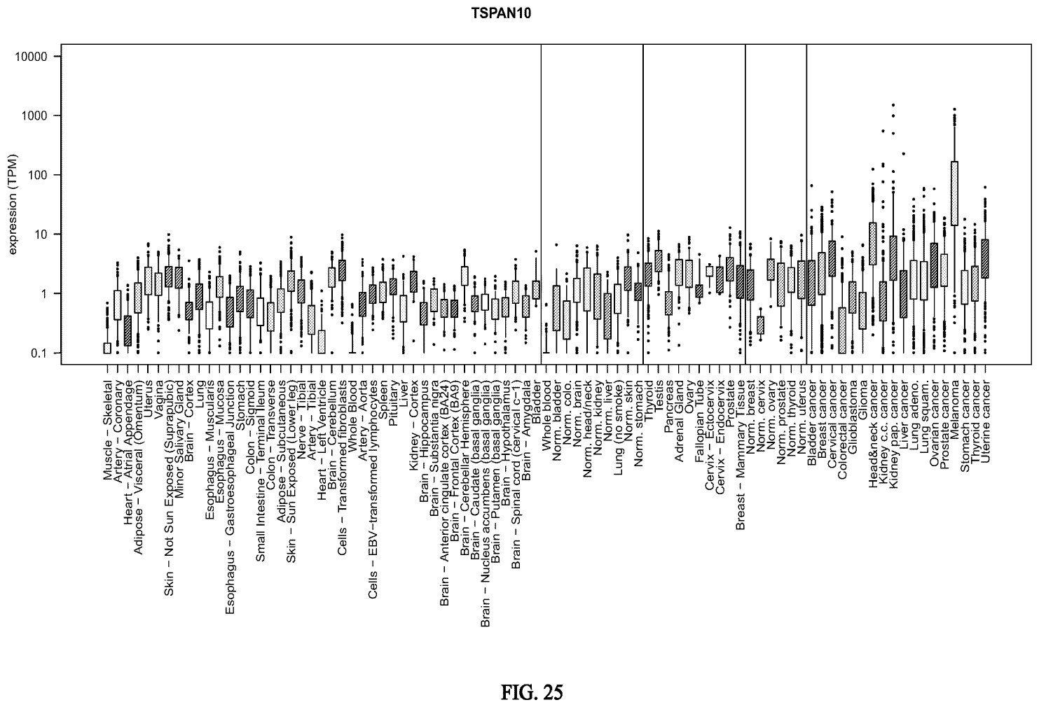

FIG. 26 is a boxplot illustrating expression levels of gene PAGE5 in a number of different normal tissues and tumors.

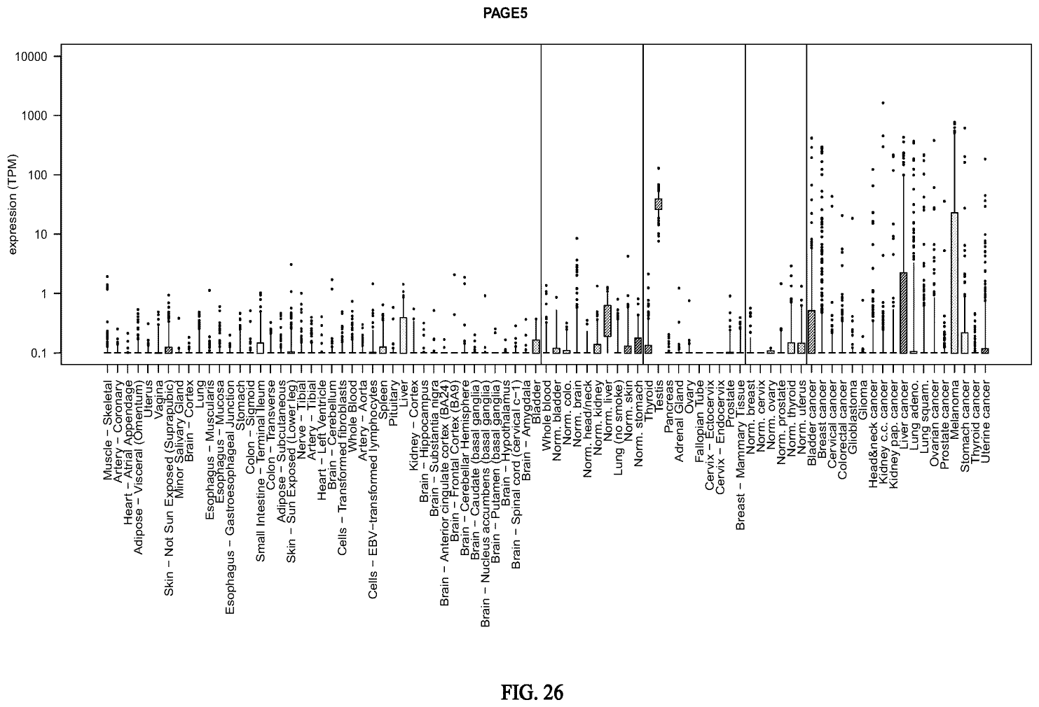

FIG. 27 is a boxplot illustrating expression levels of gene CSAG1 in a number of different normal tissues and tumors.

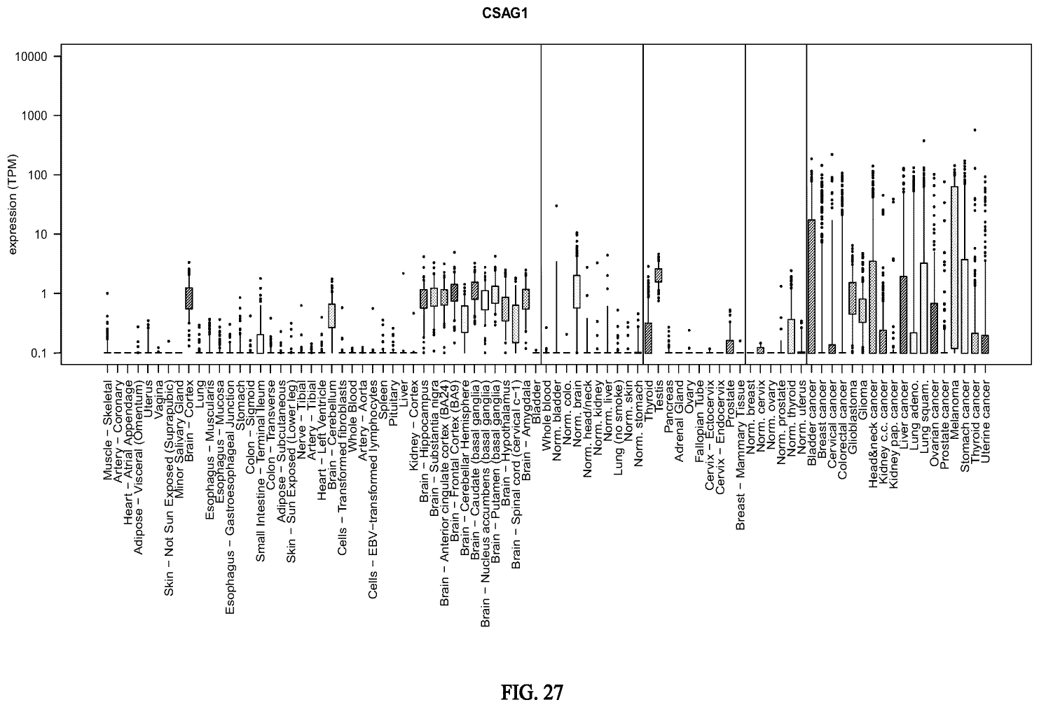

FIG. 28 is a boxplot illustrating expression levels of gene PRDM7 in a number of different normal tissues and tumors.

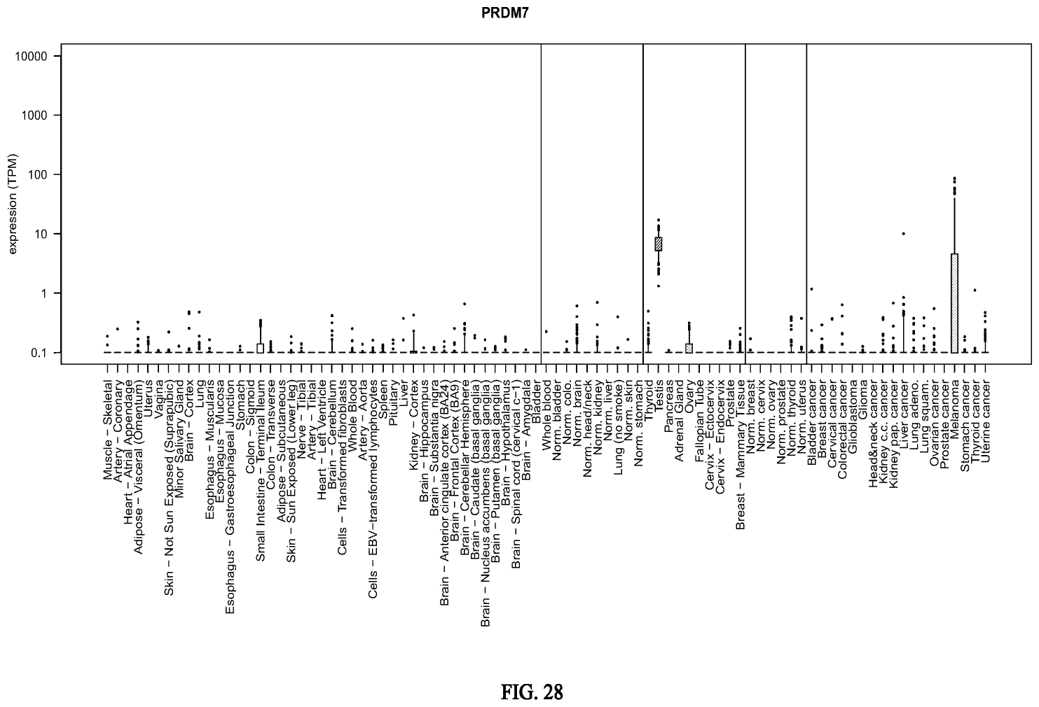

FIG. 29 is a boxplot illustrating expression levels of gene TG in a number of different normal tissues and tumors.

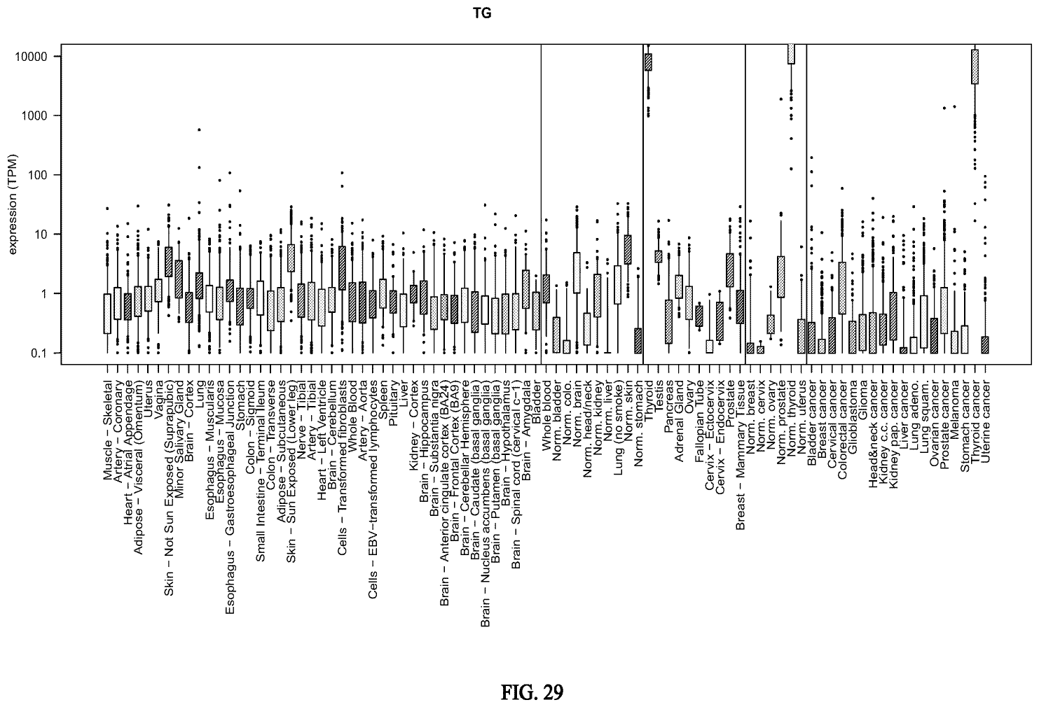

FIG. 30 is a boxplot illustrating expression levels of gene TSHR in a number of different normal tissues and tumors.

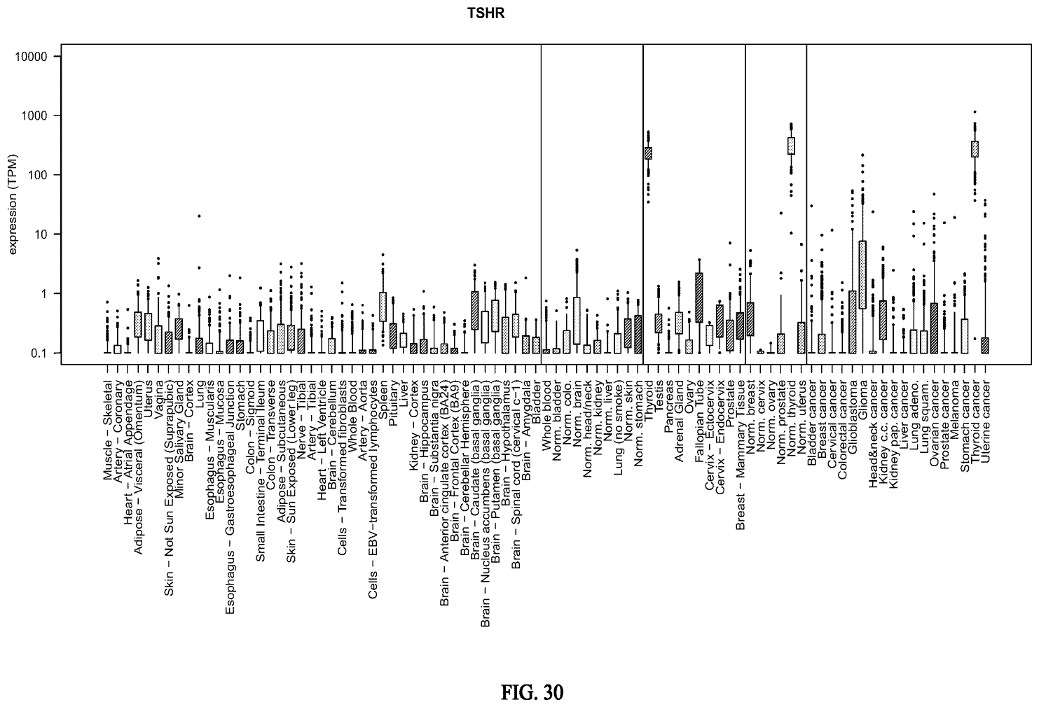

FIG. 31 is a boxplot illustrating expression levels of gene RSPH6A in a number of different normal tissues and tumors.

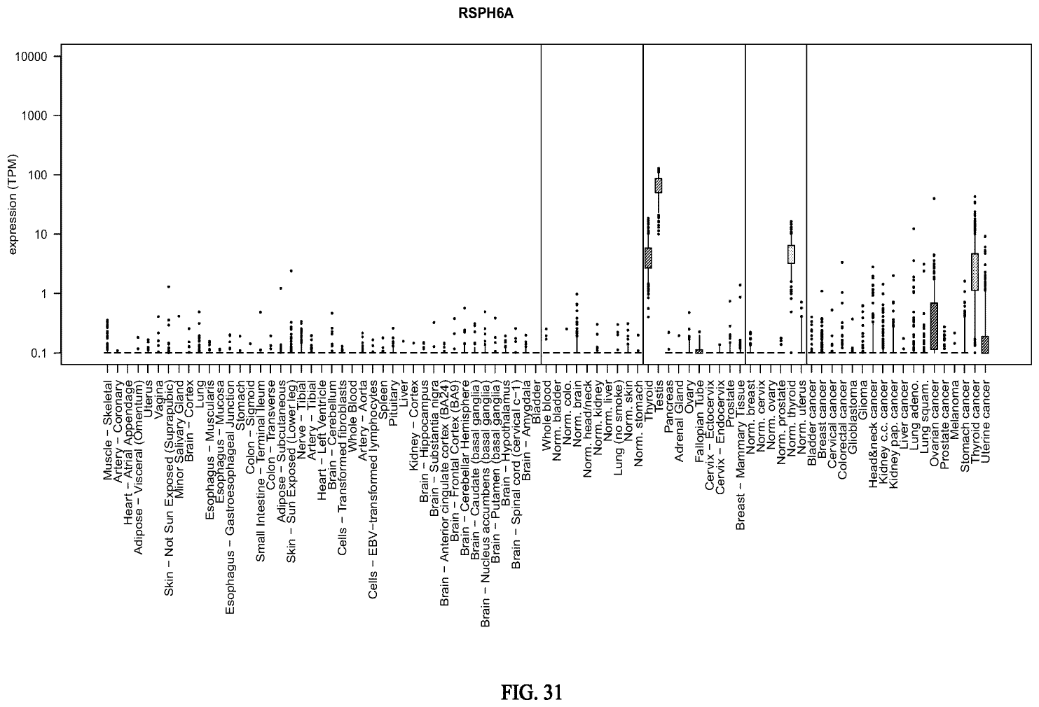

FIG. 32 is a boxplot illustrating expression levels of gene SCXB in a number of different normal tissues and tumors.

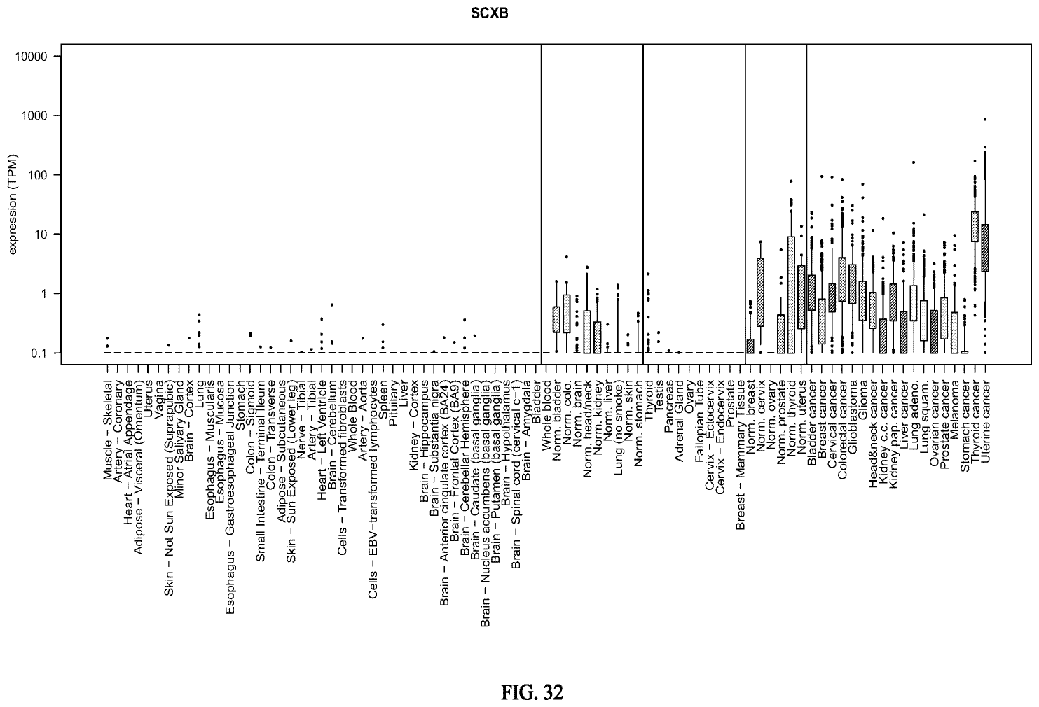

FIG. 33 is a boxplot illustrating expression levels of gene HIST1H4K in a number of different normal tissues and tumors.

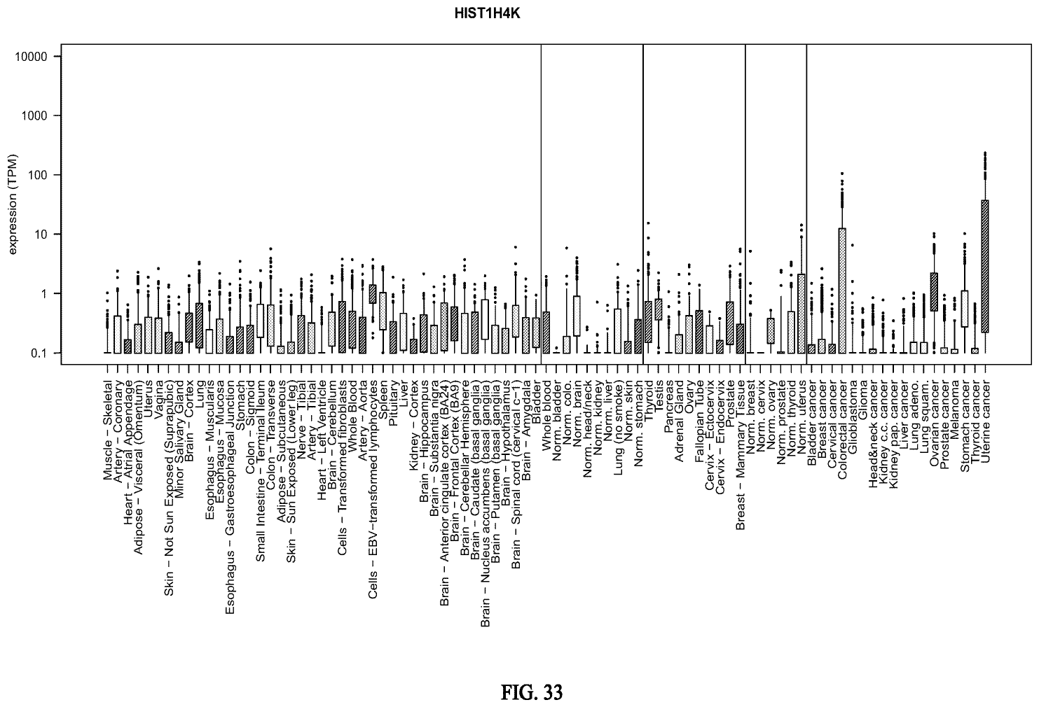

FIG. 34 is a boxplot illustrating expression levels of gene ALPPL2 in a number of different normal tissues and tumors.

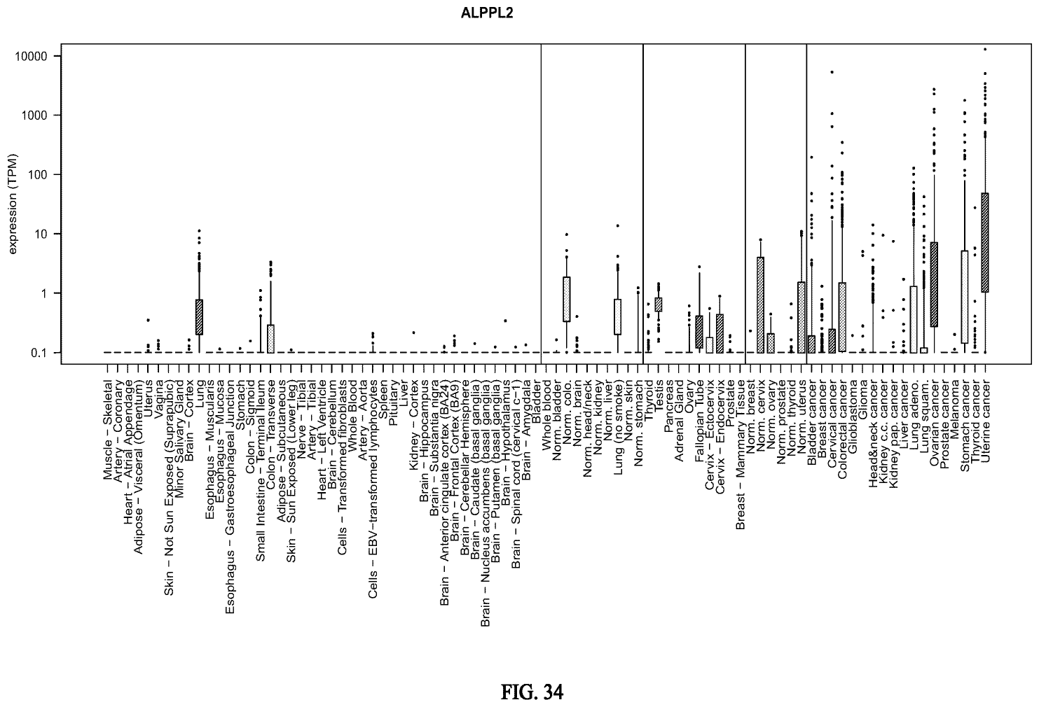

FIG. 35 is a boxplot illustrating expression levels of gene PRM2 in a number of different normal tissues and tumors.

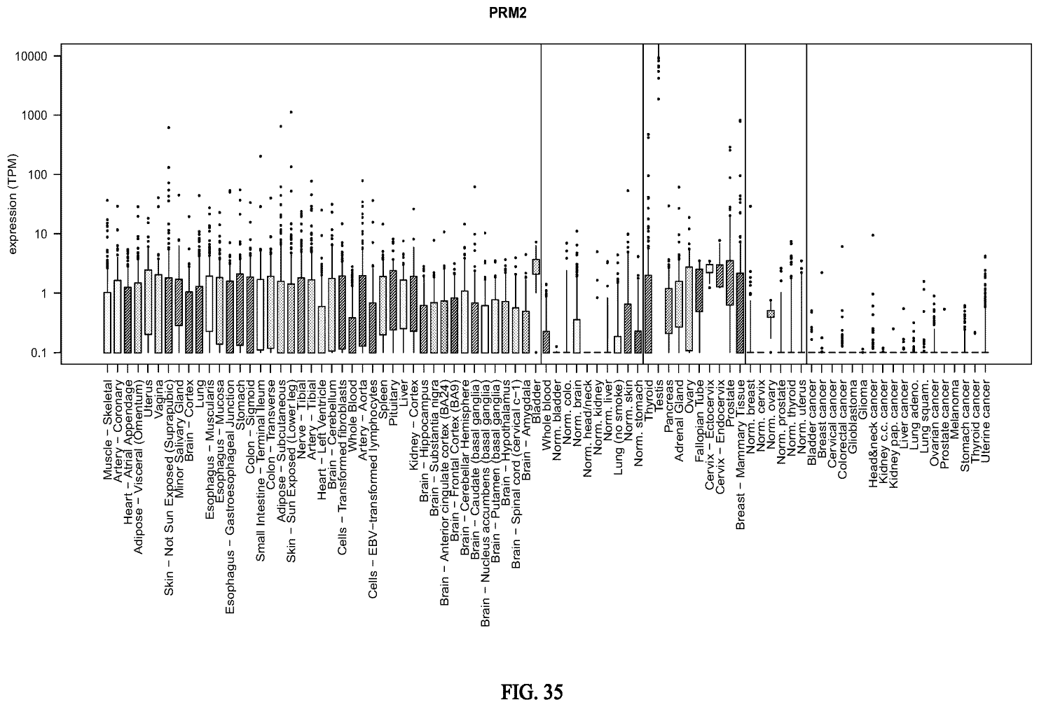

FIG. 36 is a boxplot illustrating expression levels of gene PRM1 in a number of different normal tissues and tumors.

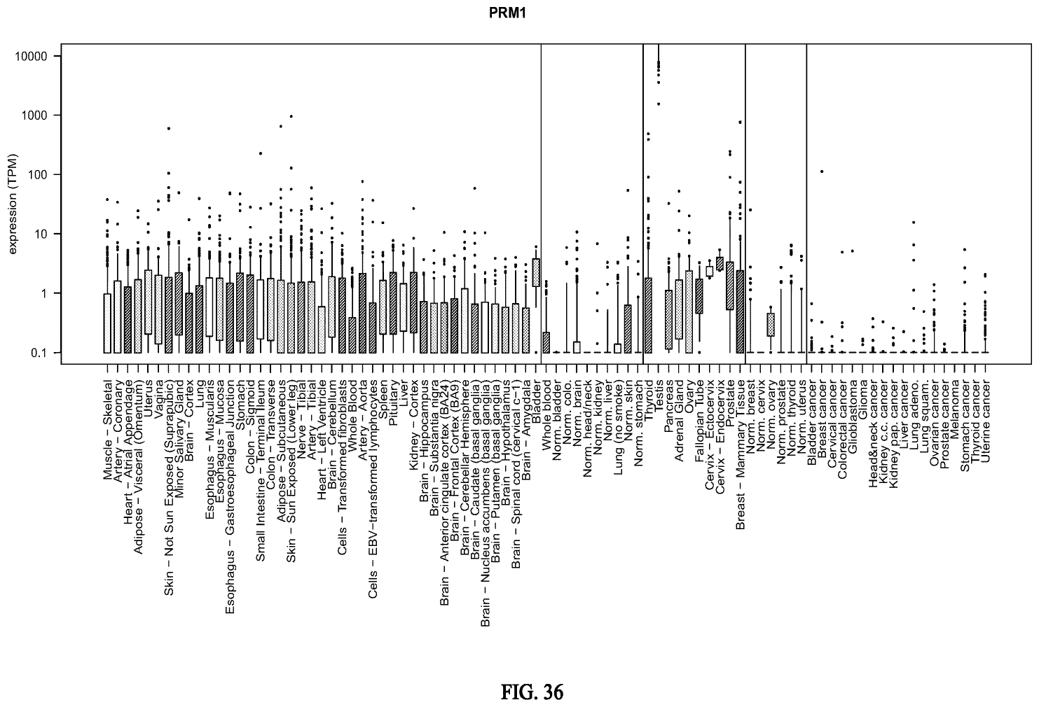

FIG. 37 is a boxplot illustrating expression levels of gene TNP1 in a number of different normal tissues and tumors.

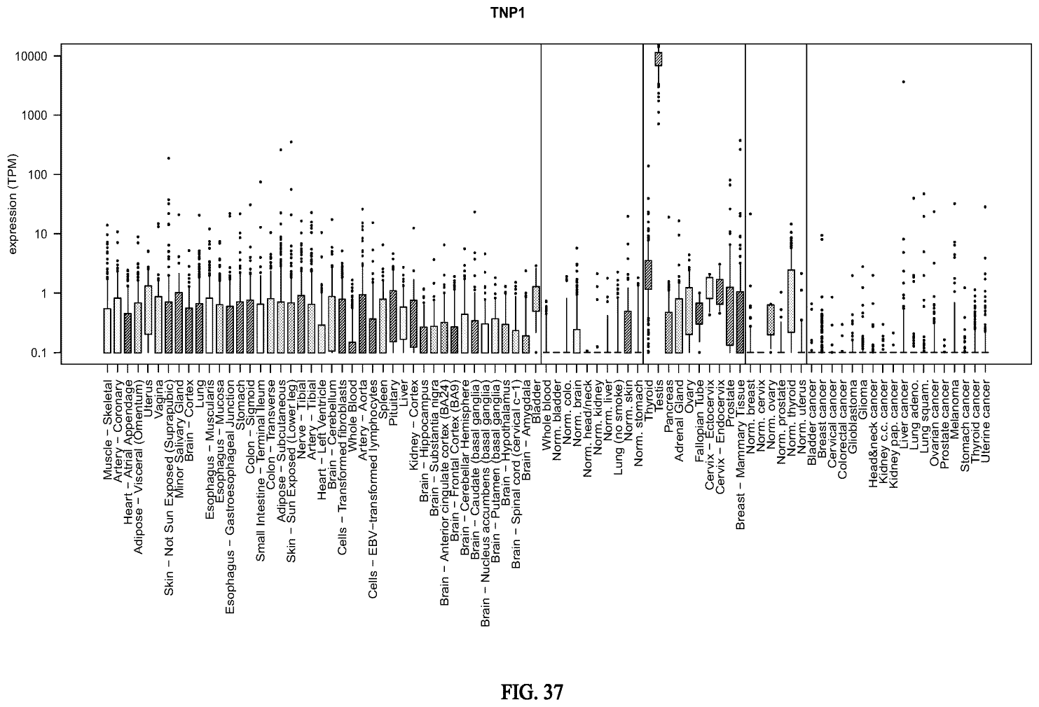

FIG. 38 is a boxplot illustrating expression levels of gene LELP1 in a number of different normal tissues and tumors.

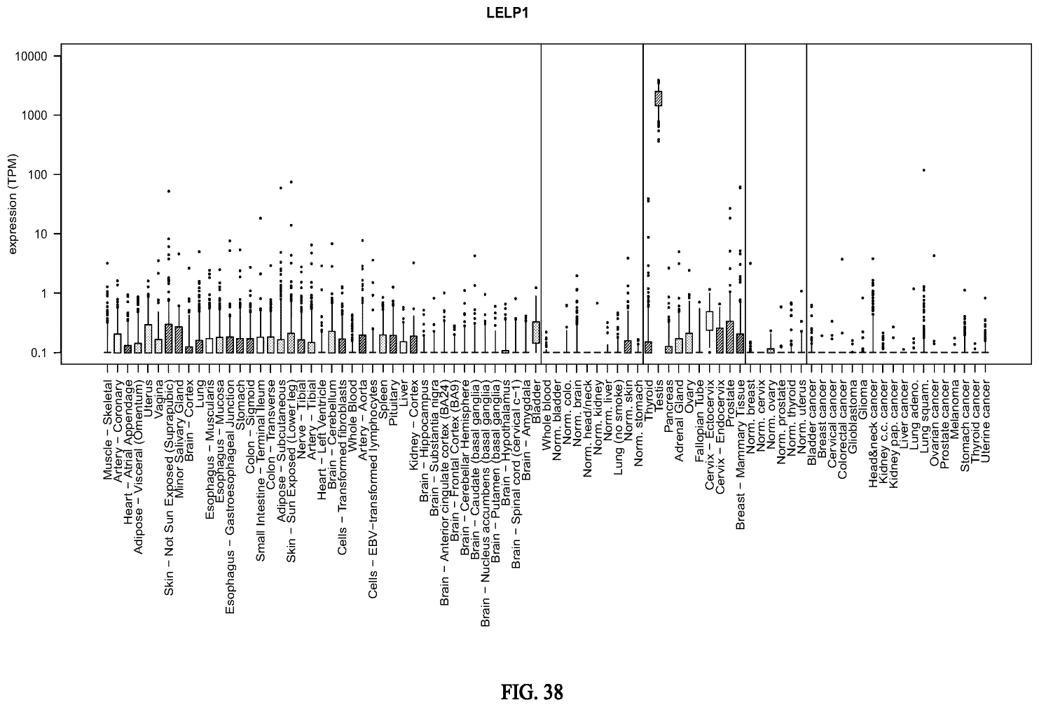

FIG. 39 is a boxplot illustrating expression levels of gene HMGB4 in a number of different normal tissues and tumors.

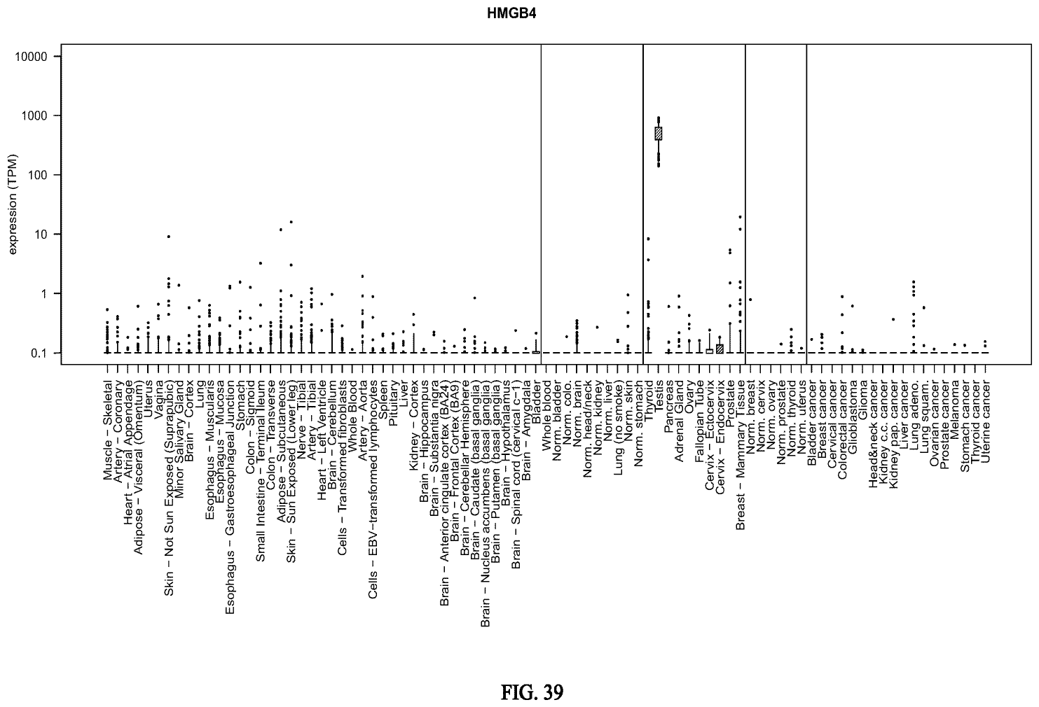

FIG. 40 is a boxplot illustrating expression levels of gene AKAP4 in a number of different normal tissues and tumors.

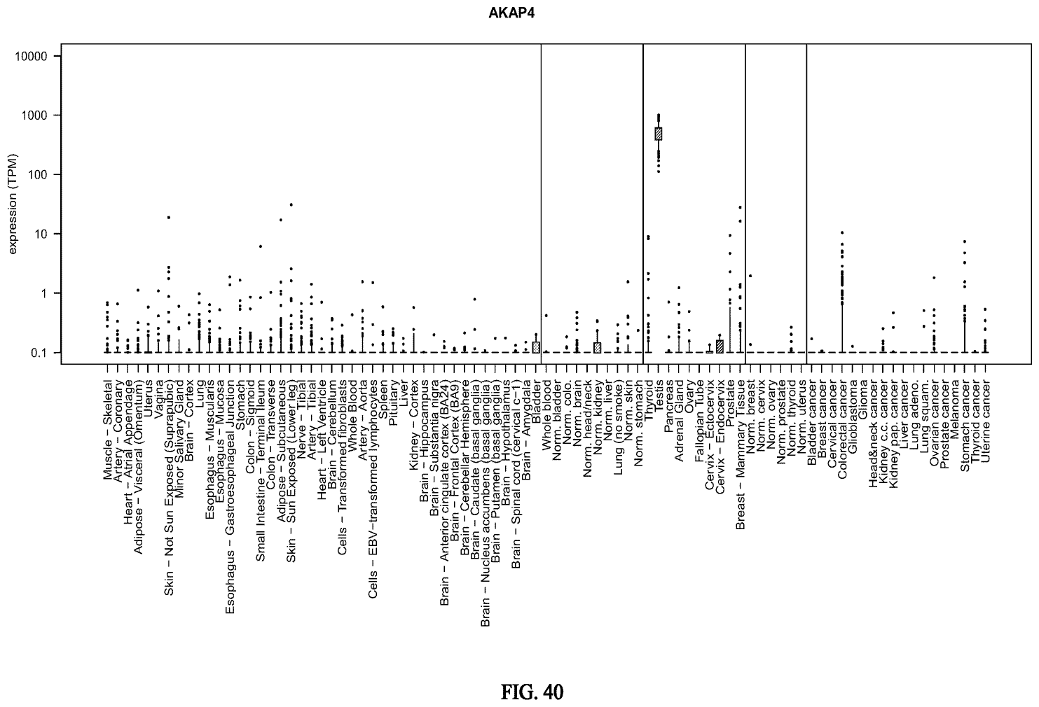

FIG. 41 is a boxplot illustrating expression levels of gene CETN1 in a number of different normal tissues and tumors.

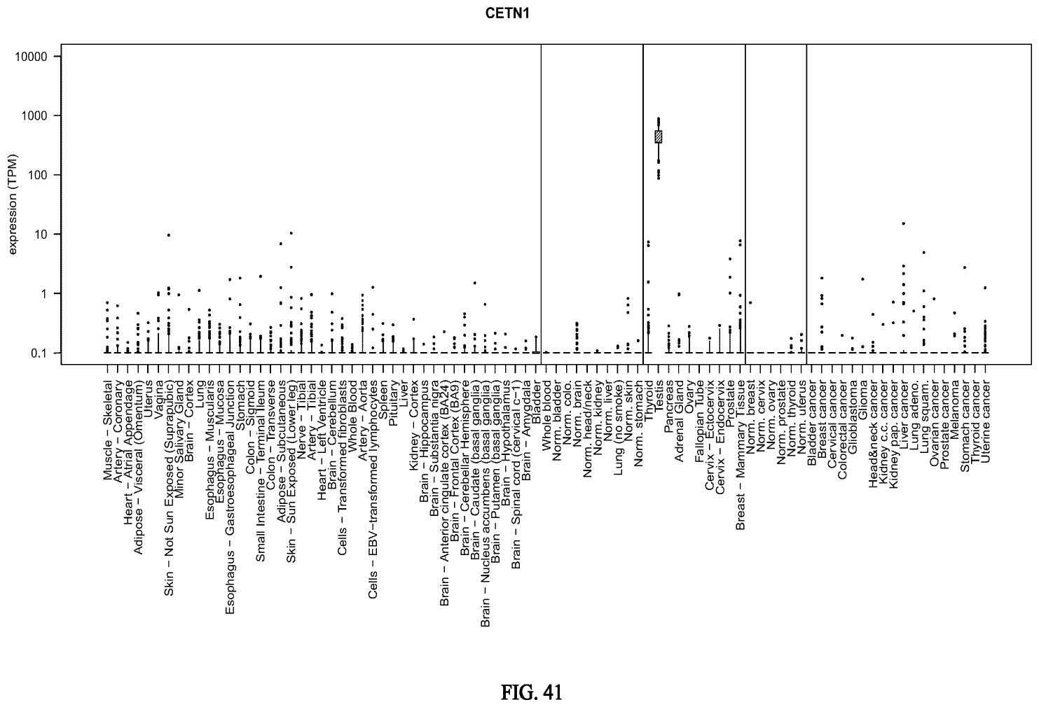

FIG. 42 is a boxplot illustrating expression levels of gene UBQLN3 in a number of different normal tissues and tumors.

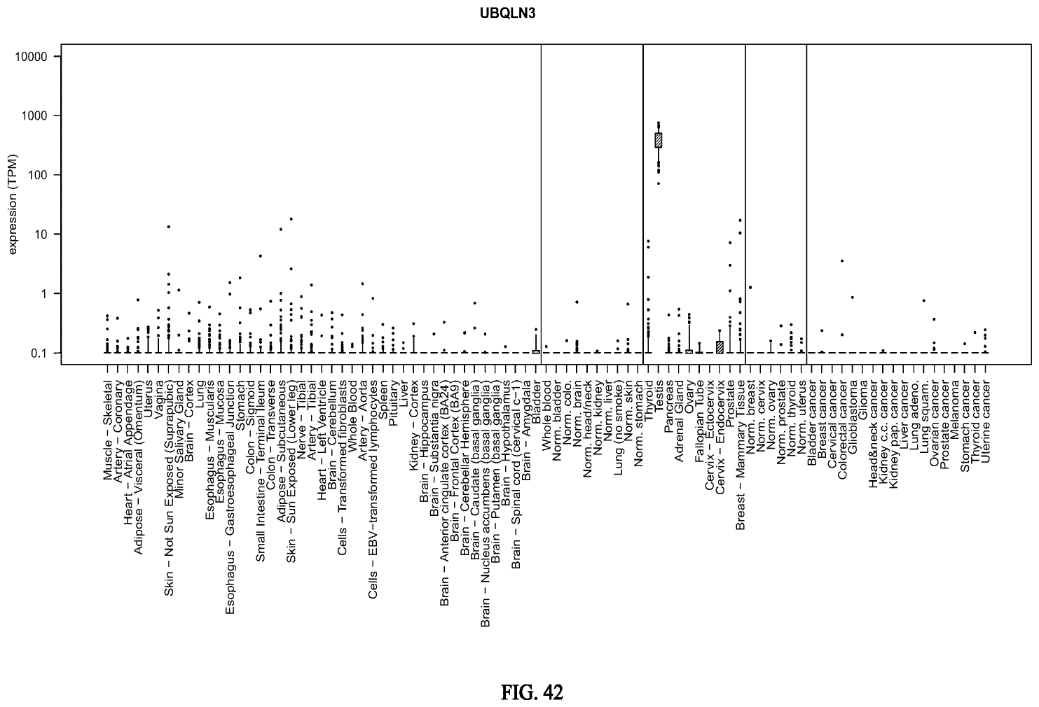

FIG. 43 is a boxplot illustrating expression levels of gene ACTL7A in a number of different normal tissues and tumors.

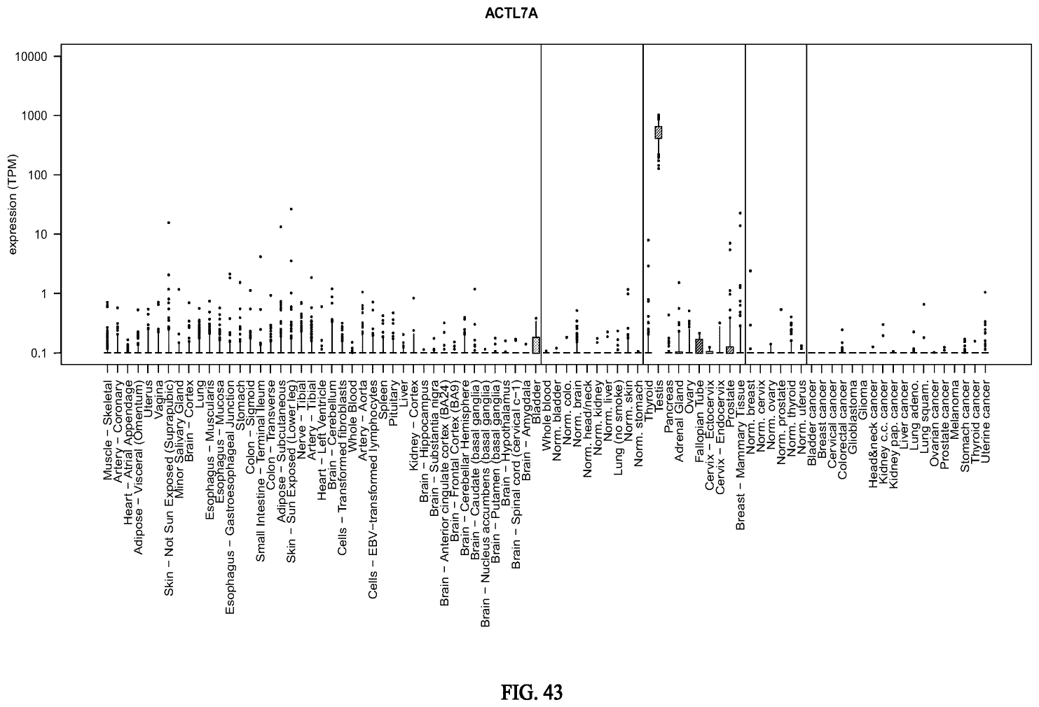

FIG. 44 is a boxplot illustrating expression levels of gene ACTL9 in a number of different normal tissues and tumors.

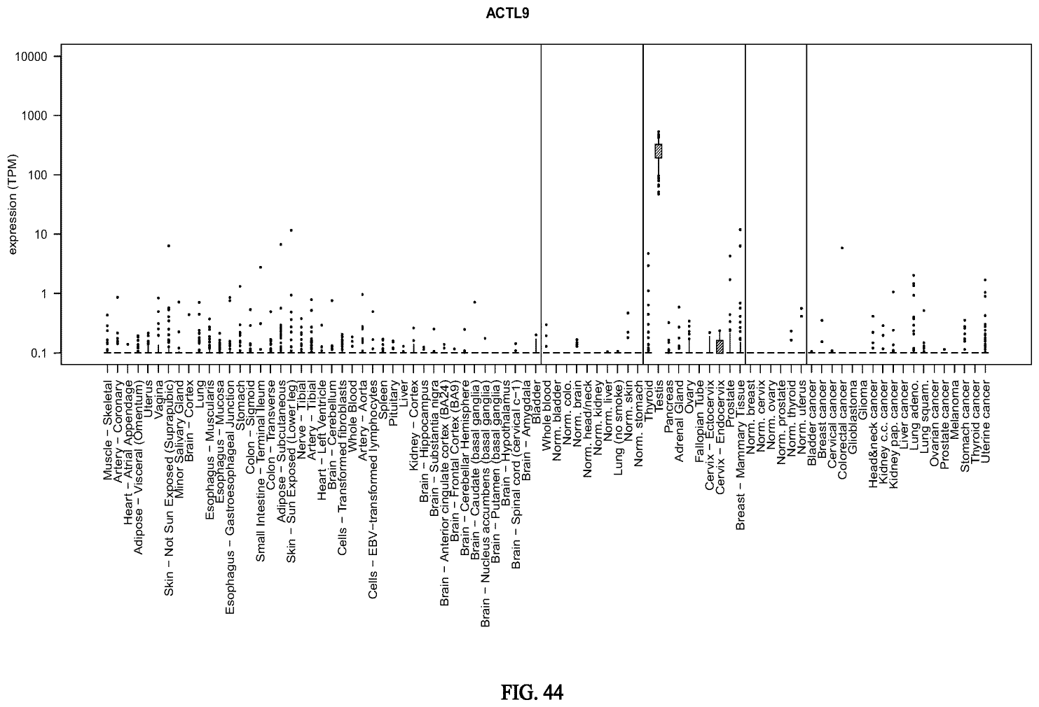

FIG. 45 is a boxplot illustrating expression levels of gene ACTRT2 in a number of different normal tissues and tumors.

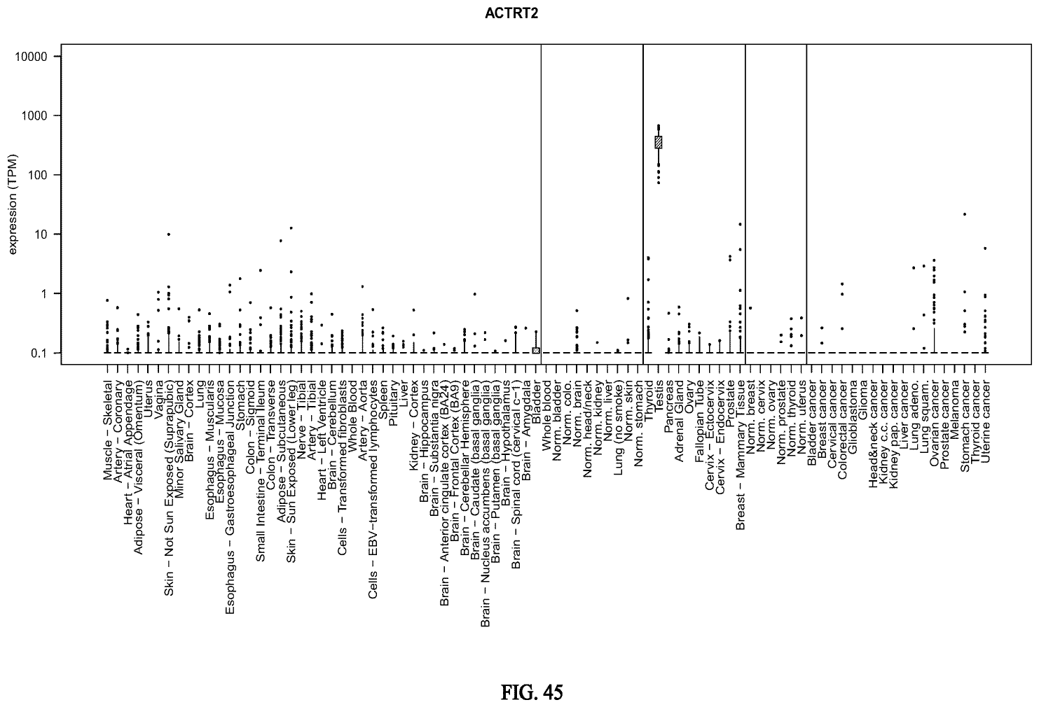

FIG. 46 is a boxplot illustrating expression levels of gene PGK2 in a number of different normal tissues and tumors.

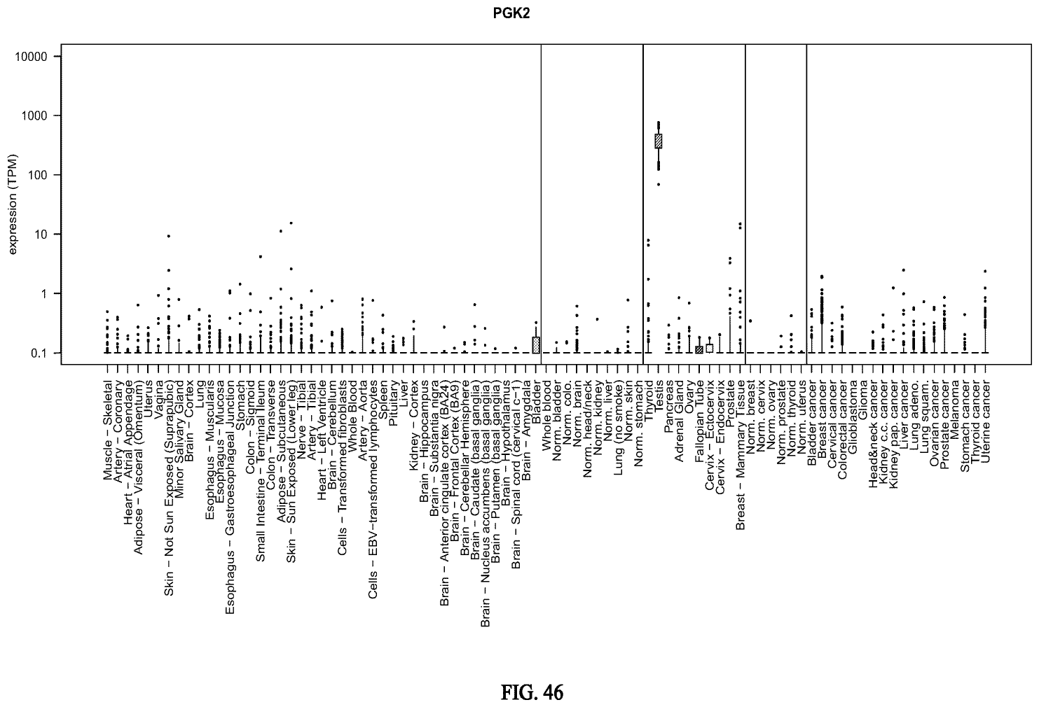

FIG. 47 is a boxplot illustrating expression levels of gene C2orf53 in a number of different normal tissues and tumors.

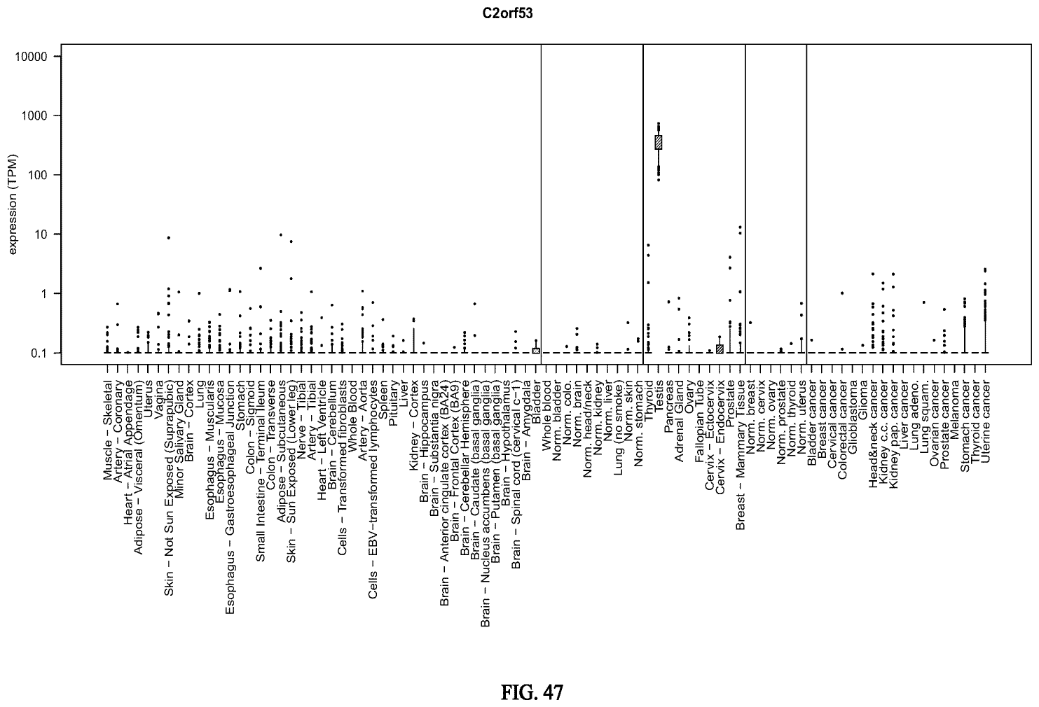

FIG. 48 is a boxplot illustrating expression levels of gene KIF2B in a number of different normal tissues and tumors.

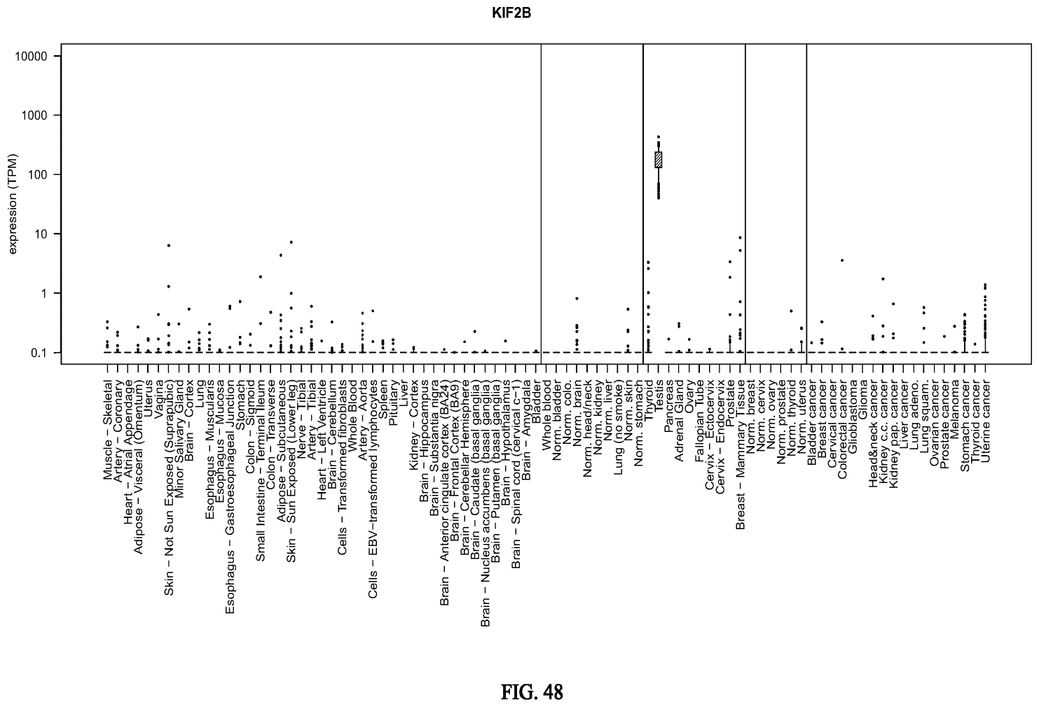

FIG. 49 is a boxplot illustrating expression levels of gene ADAD1 in a number of different normal tissues and tumors.

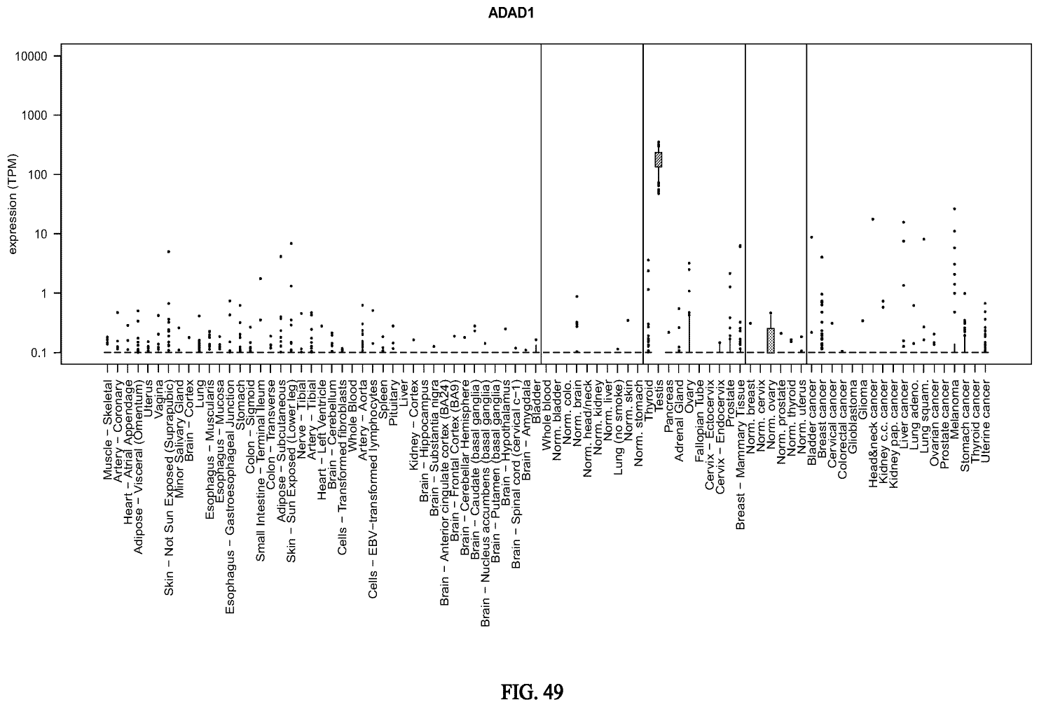

FIG. 50 is a boxplot illustrating expression levels of gene SPATA8 in a number of different normal tissues and tumors.

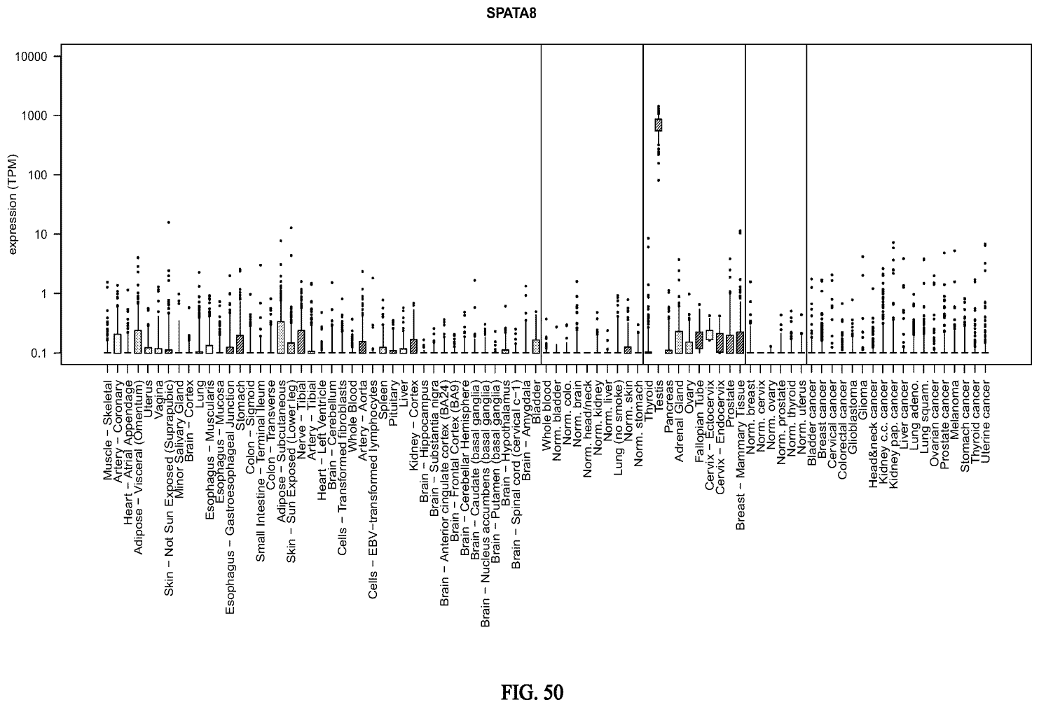

FIG. 51 is a boxplot illustrating expression levels of gene CCDC70 in a number of different normal tissues and tumors.

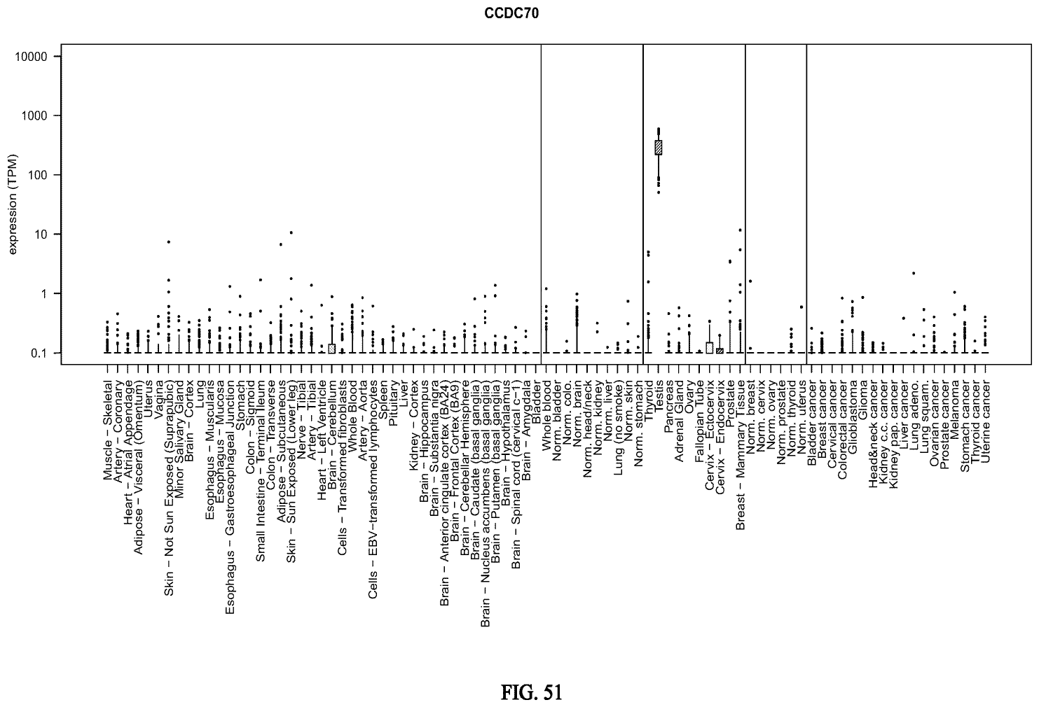

FIG. 52 is a boxplot illustrating expression levels of gene TPD52L3 in a number of different normal tissues and tumors.

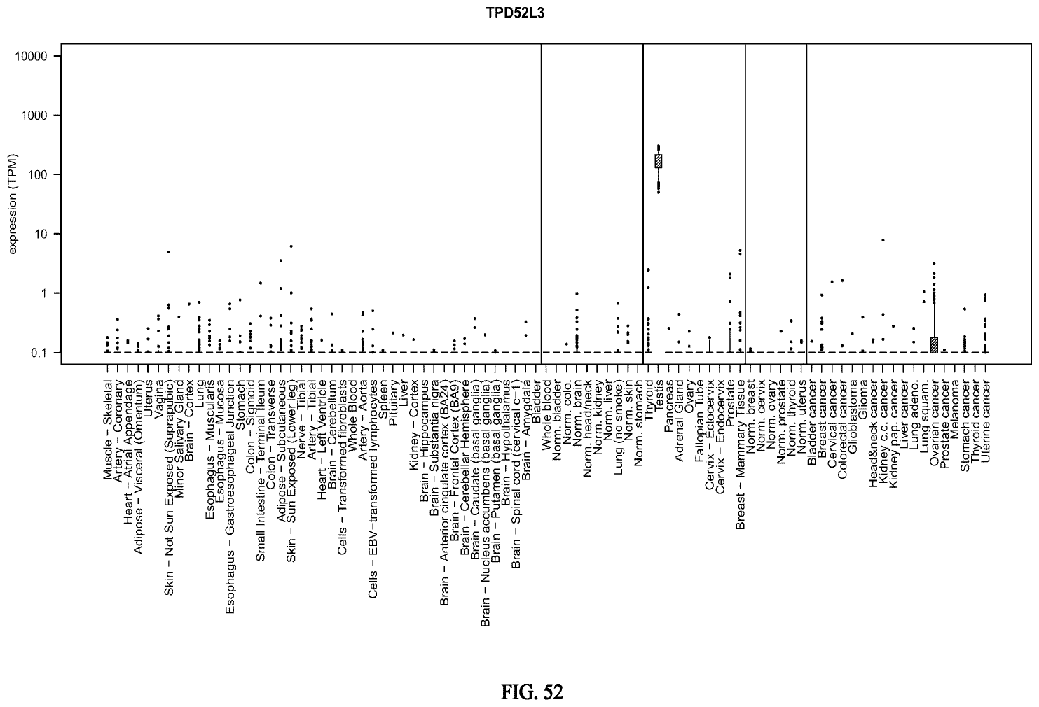

FIG. 53 is a boxplot illustrating expression levels of gene ACTL7B in a number of different normal tissues and tumors.

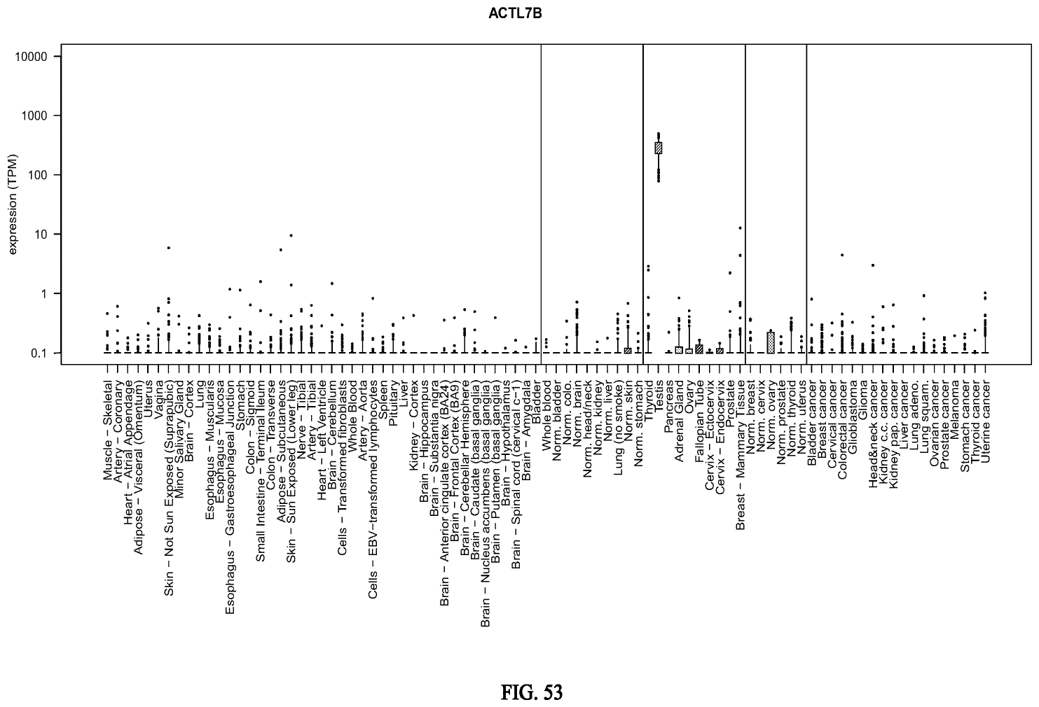

FIG. 54 is a boxplot illustrating expression levels of gene DMRTB1 in a number of different normal tissues and tumors.

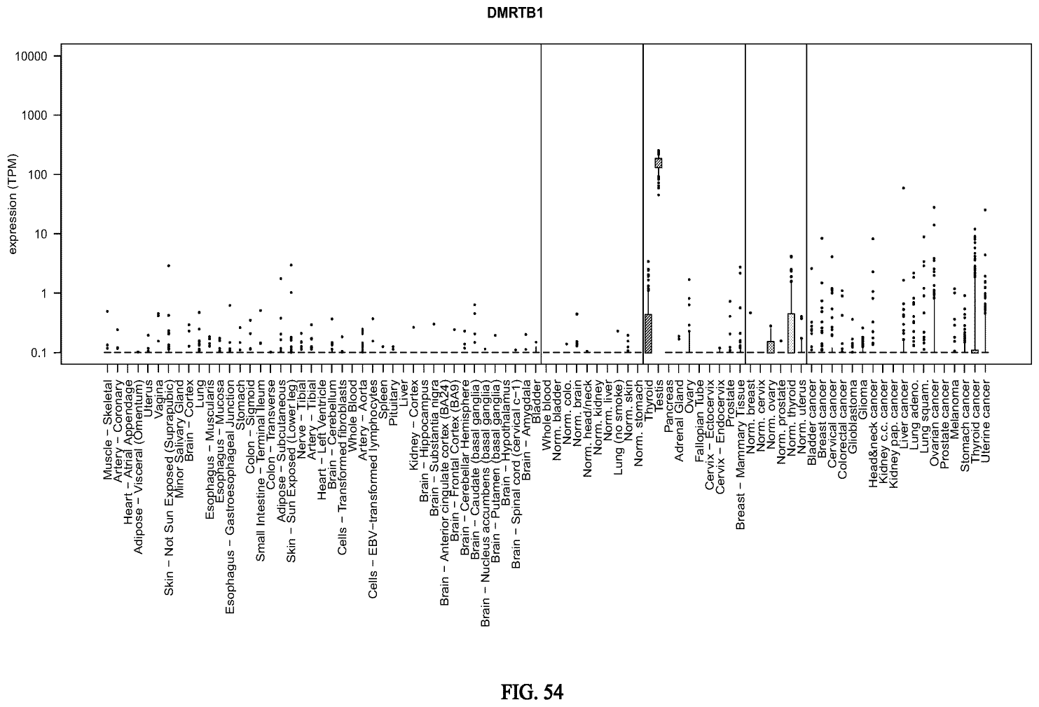

FIG. 55 is a boxplot illustrating expression levels of gene SYCN in a number of different normal tissues and tumors.

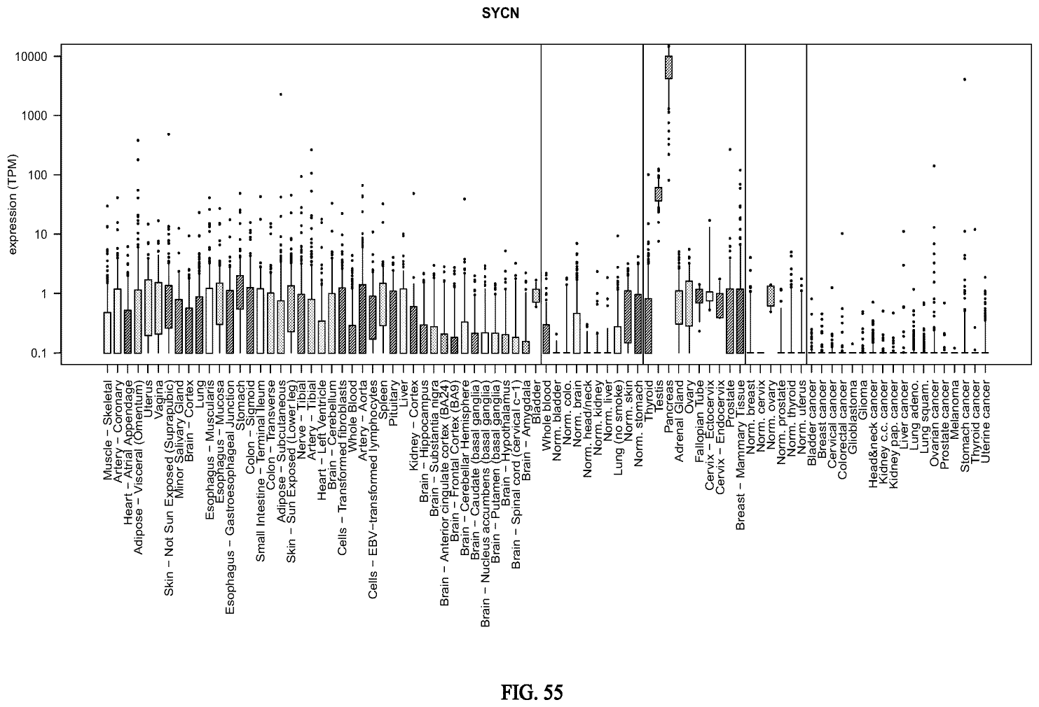

FIG. 56 is a boxplot illustrating expression levels of gene CELA2A in a number of different normal tissues and tumors.

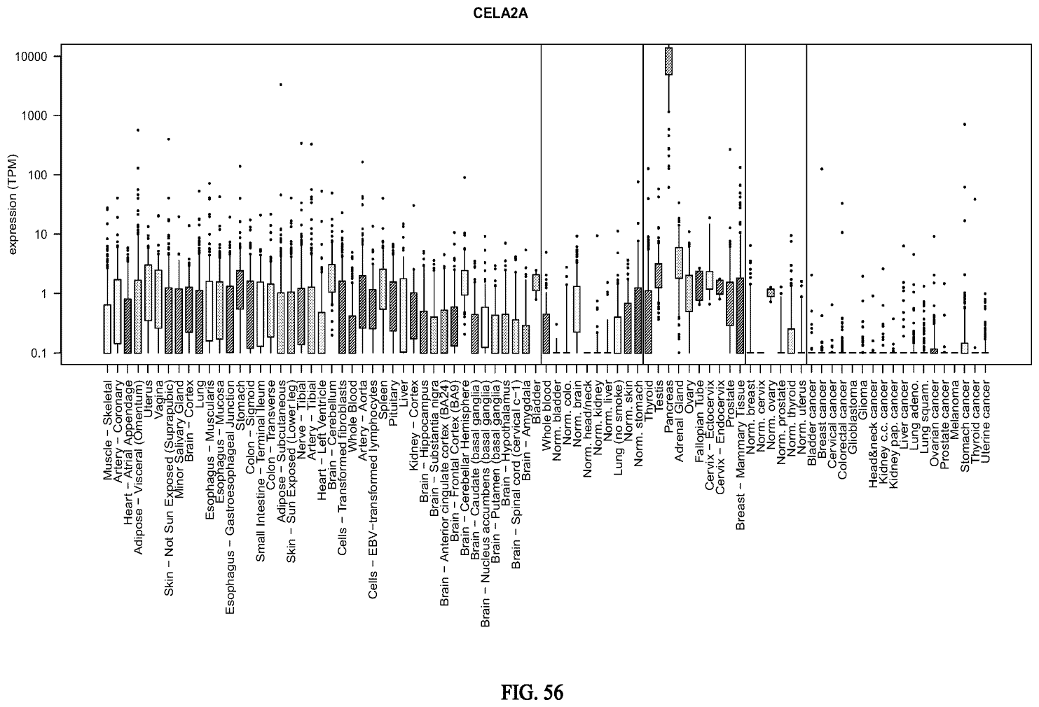

FIG. 57 is a boxplot illustrating expression levels of gene CELA2B in a number of different normal tissues and tumors.

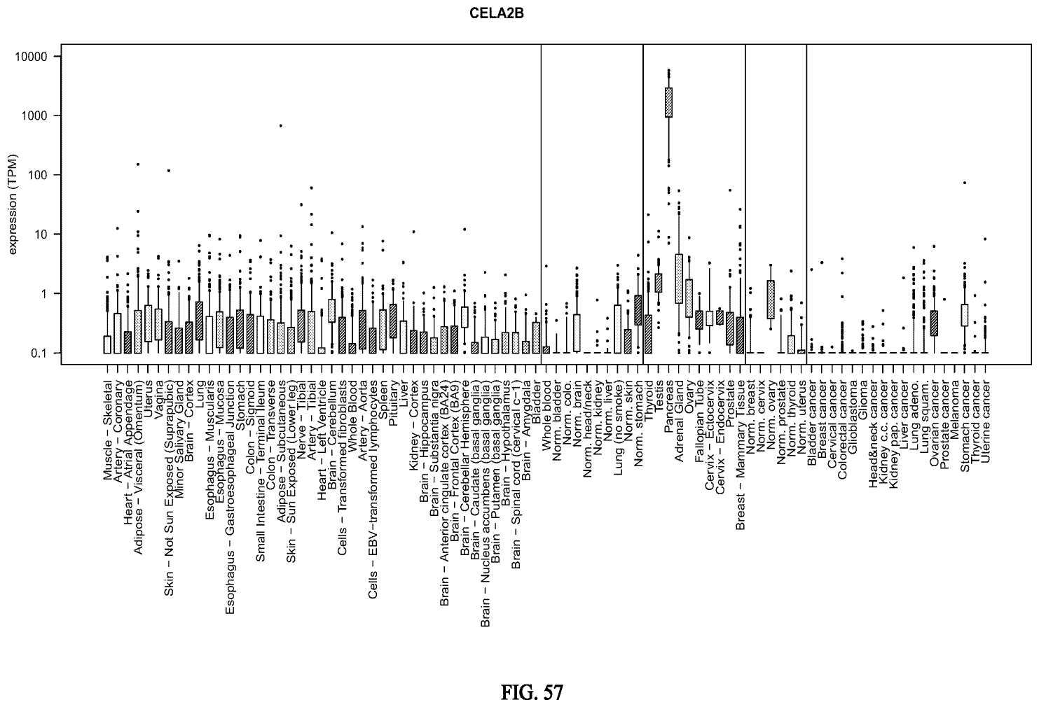

FIG. 58 is a boxplot illustrating expression levels of gene PNLIPRP1 in a number of different normal tissues and tumors.

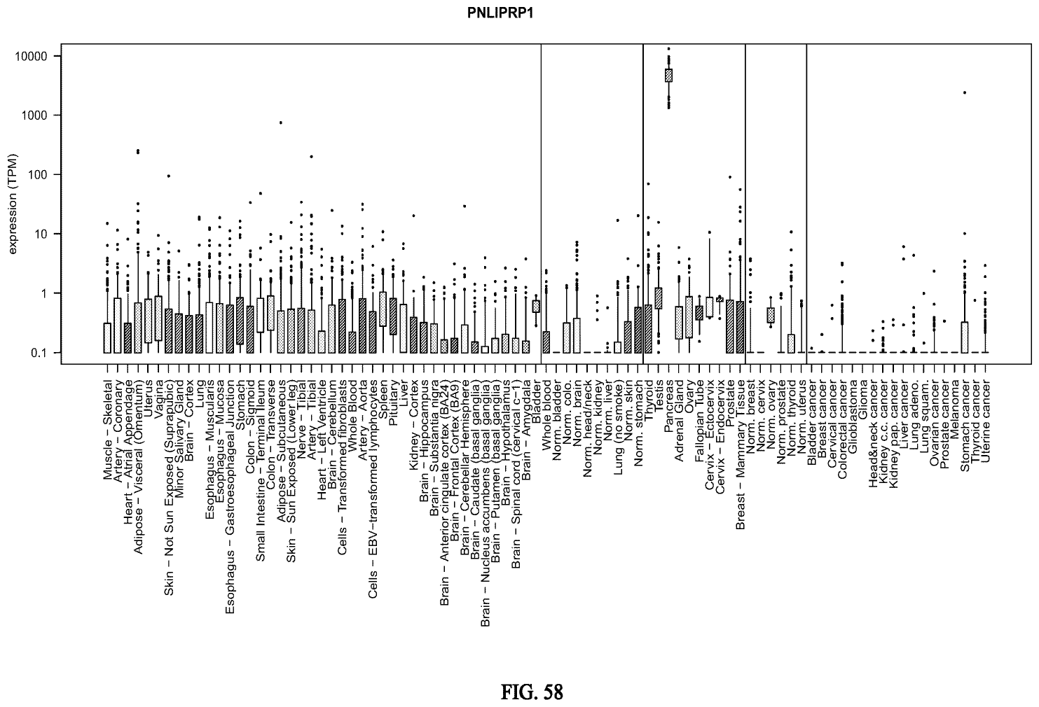

FIG. 59 is a boxplot illustrating expression levels of gene CTRC in a number of different normal tissues and tumors.

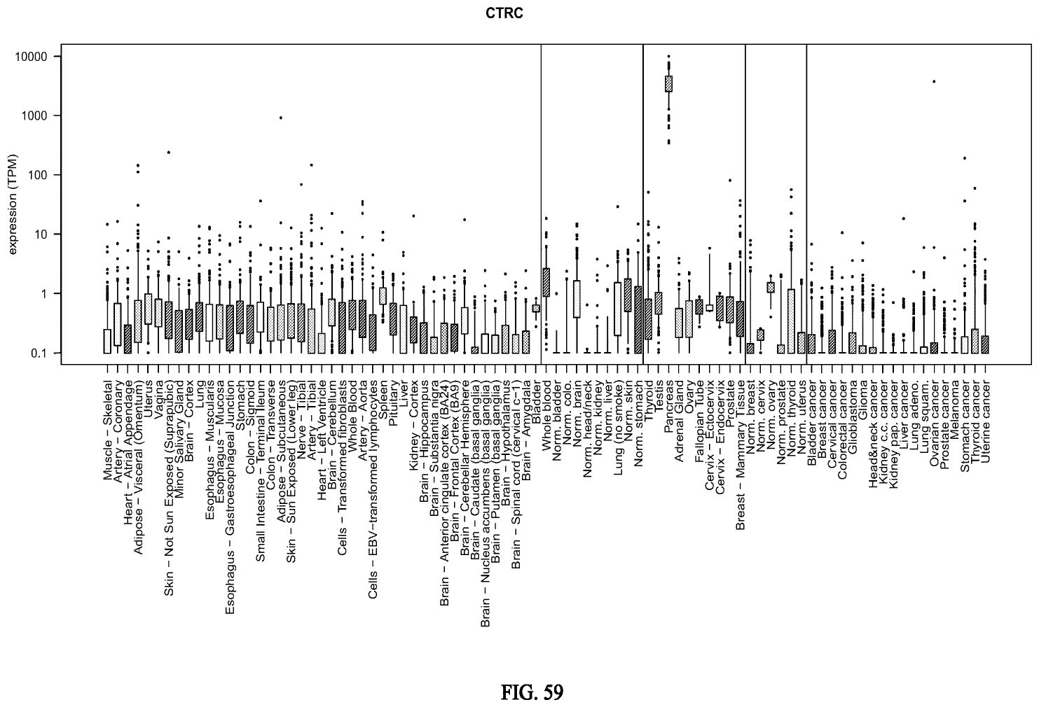

FIG. 60 is a boxplot illustrating expression levels of gene AMY2A in a number of different normal tissues and tumors.

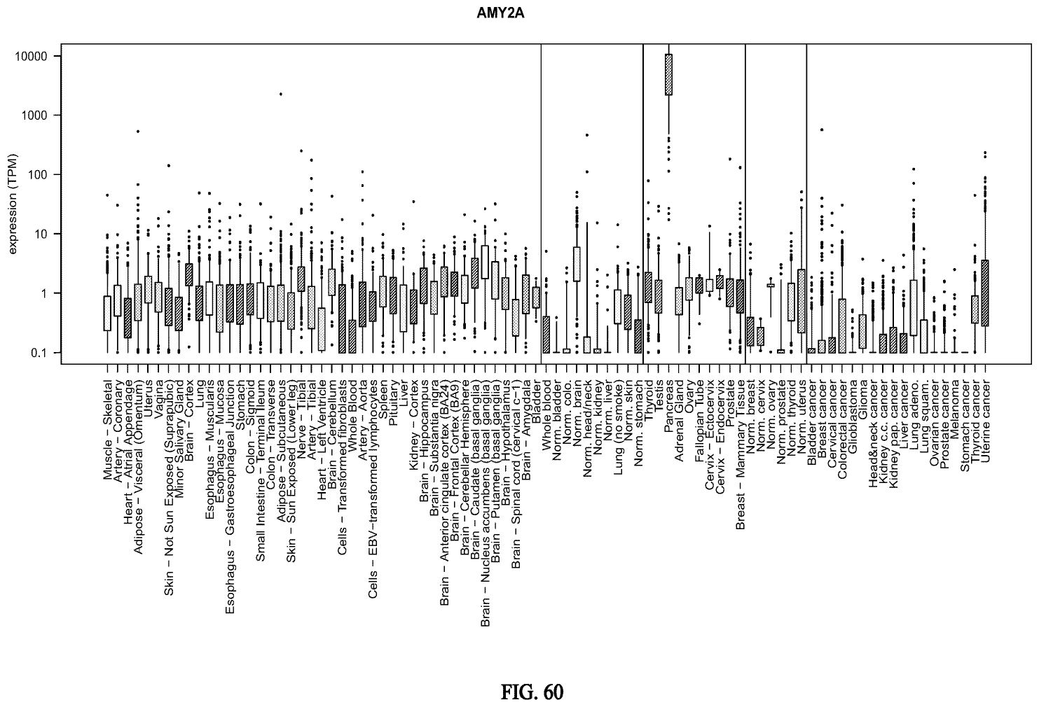

FIG. 61 is a boxplot illustrating expression levels of gene SERPINI2 in a number of different normal tissues and tumors.

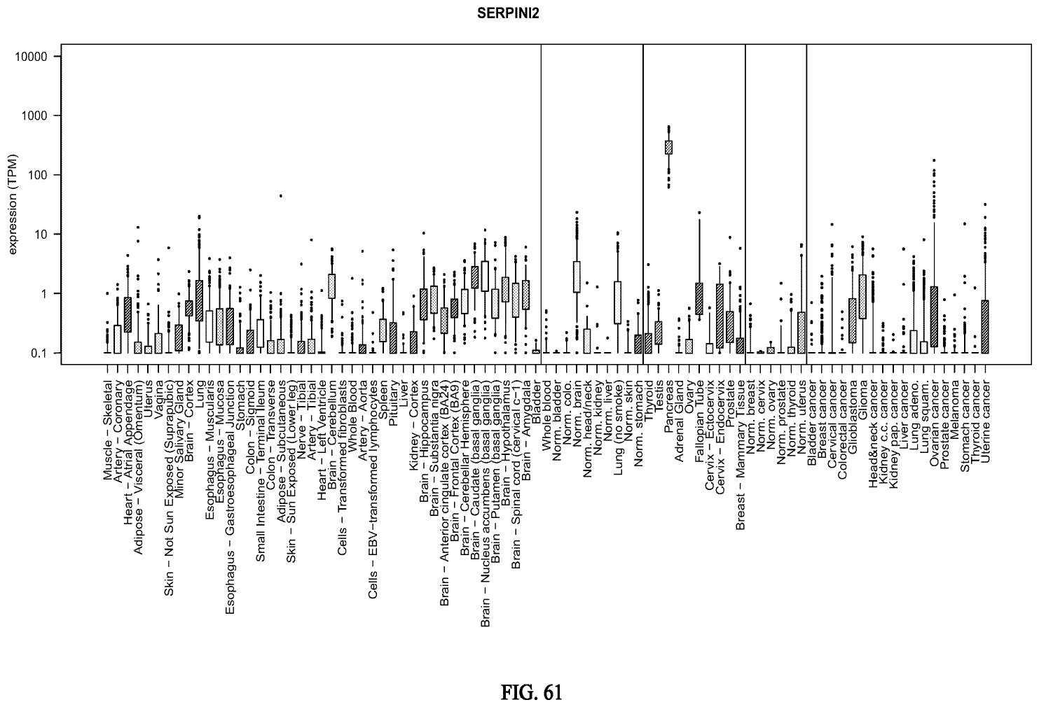

FIG. 62 is a boxplot illustrating expression levels of gene RBPJL in a number of different normal tissues and tumors.

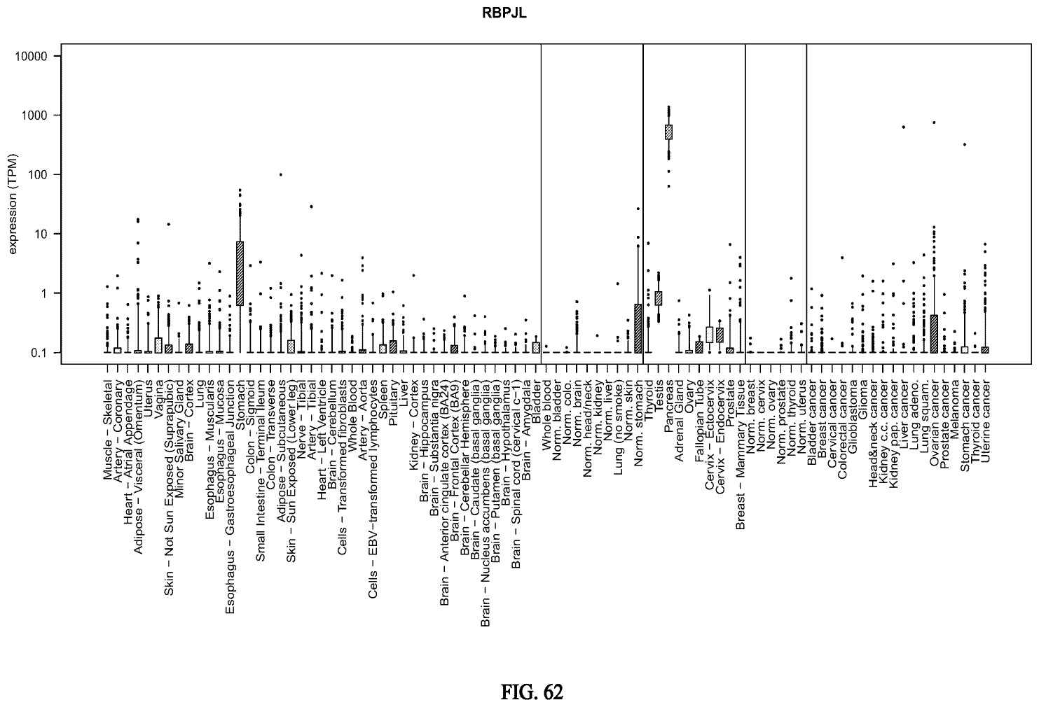

FIG. 63 is a boxplot illustrating expression levels of gene AQP12A in a number of different normal tissues and tumors.

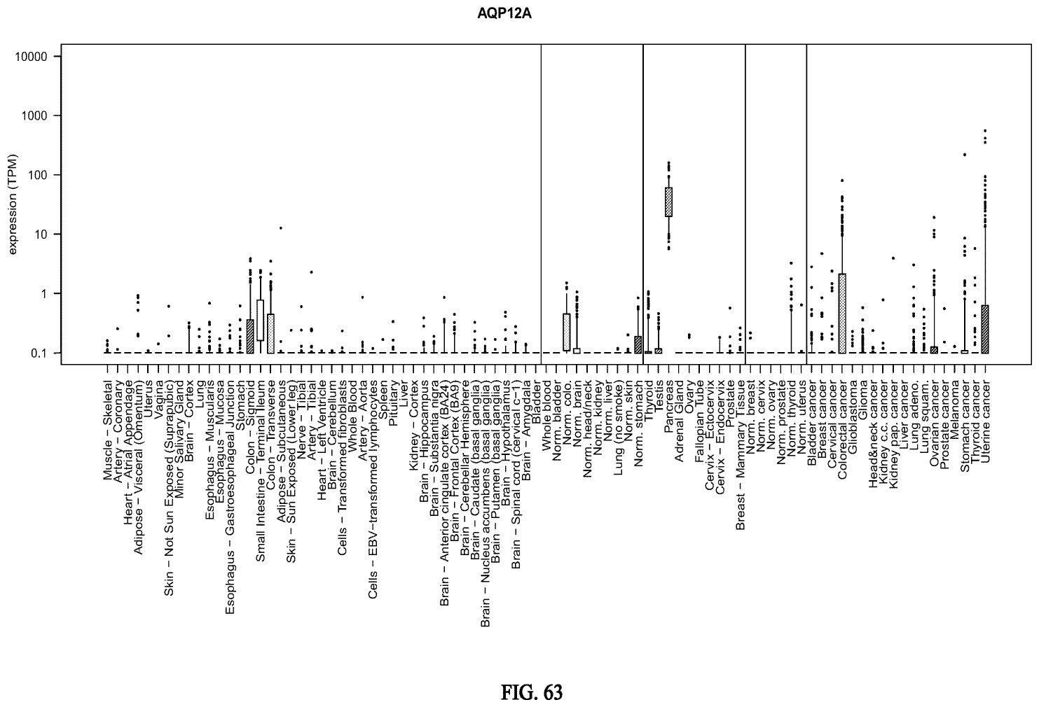

FIG. 64 is a boxplot illustrating expression levels of gene IAPP in a number of different normal tissues and tumors.

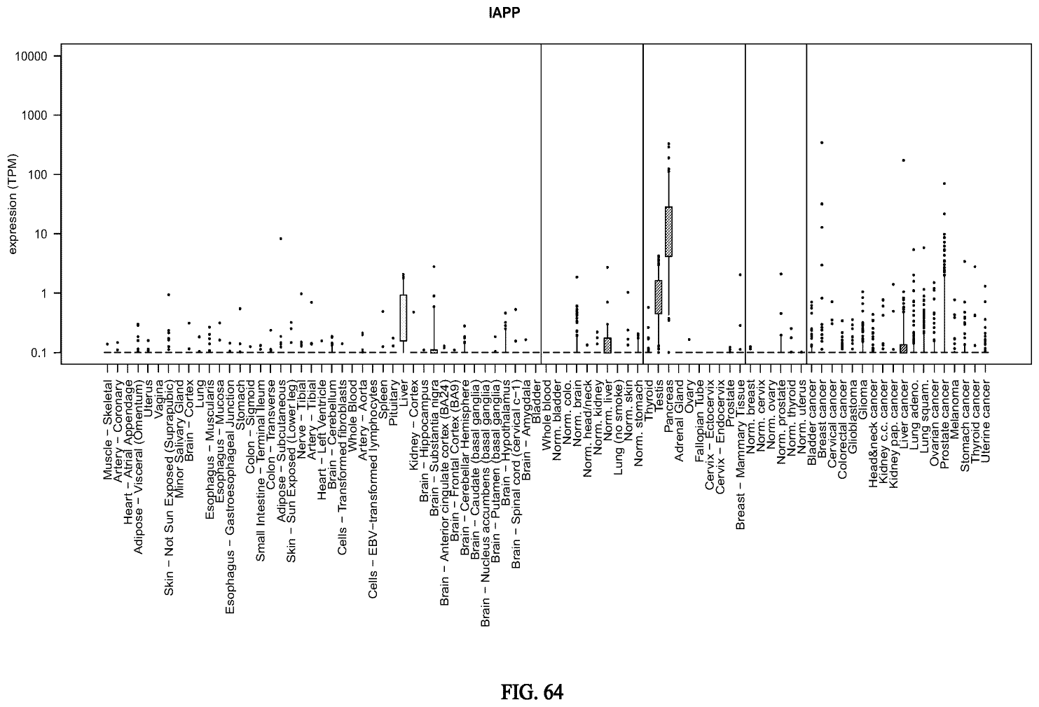

FIG. 65 is a boxplot illustrating expression levels of gene KIRREL2 in a number of different normal tissues and tumors.

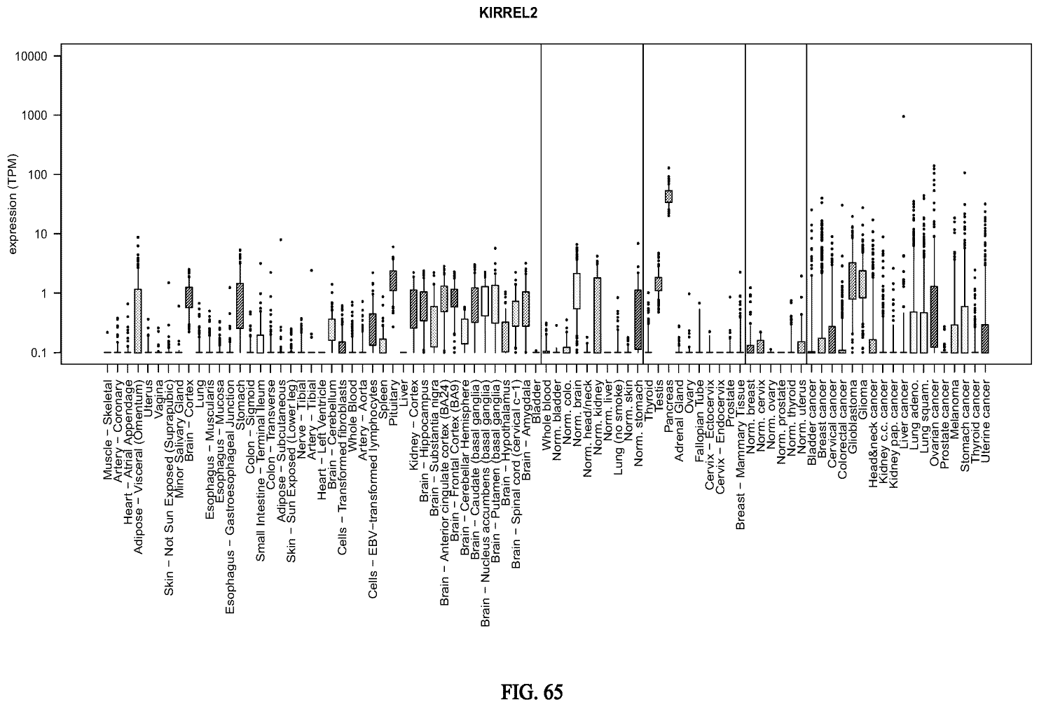

FIG. 66 is a boxplot illustrating expression levels of gene G6PC2 in a number of different normal tissues and tumors.

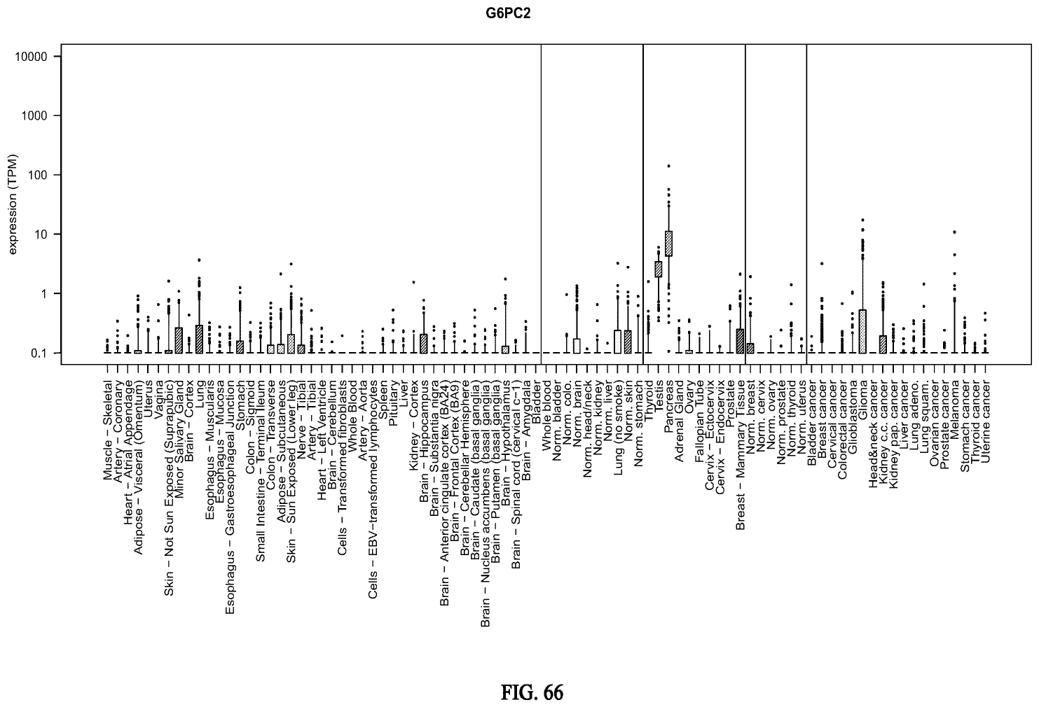

FIG. 67 is a boxplot illustrating expression levels of gene AQP12B in a number of different normal tissues and tumors.

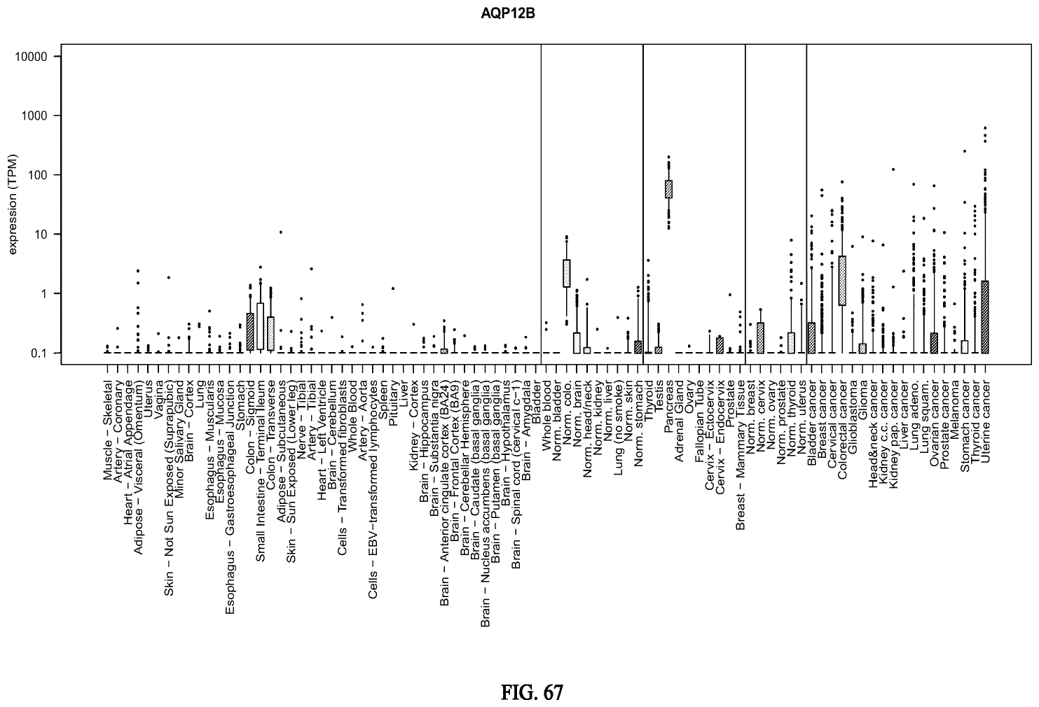

FIG. 68 is a boxplot illustrating expression levels of gene CYP11B1 in a number of different normal tissues and tumors.

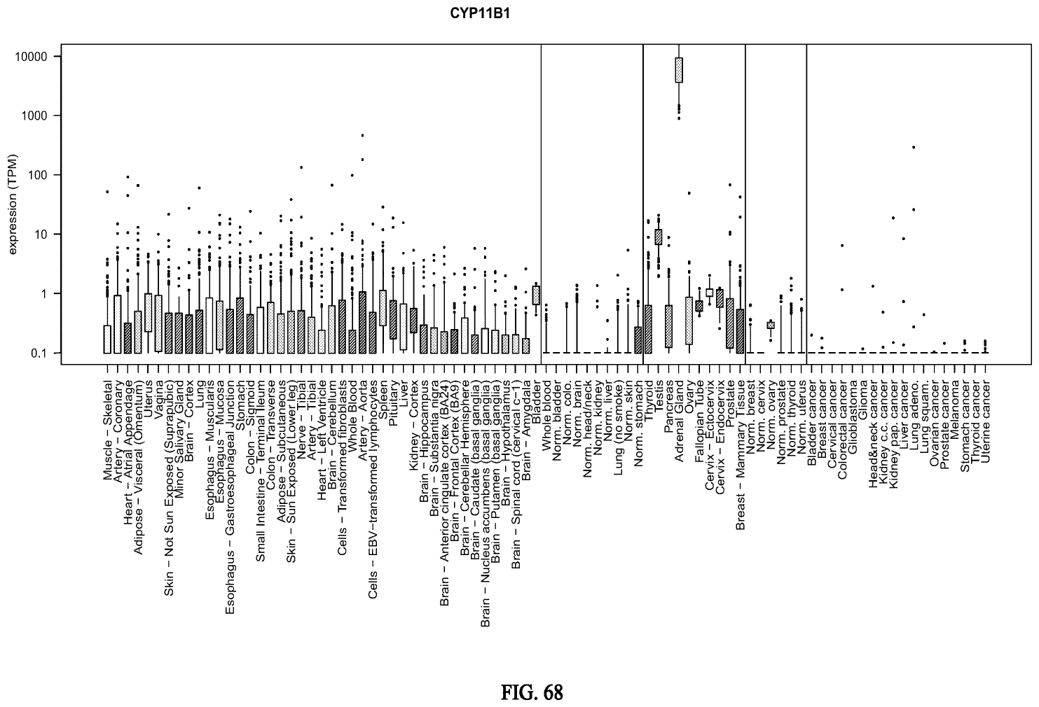

FIG. 69 is a boxplot illustrating expression levels of gene CYP11B2 in a number of different normal tissues and tumors.

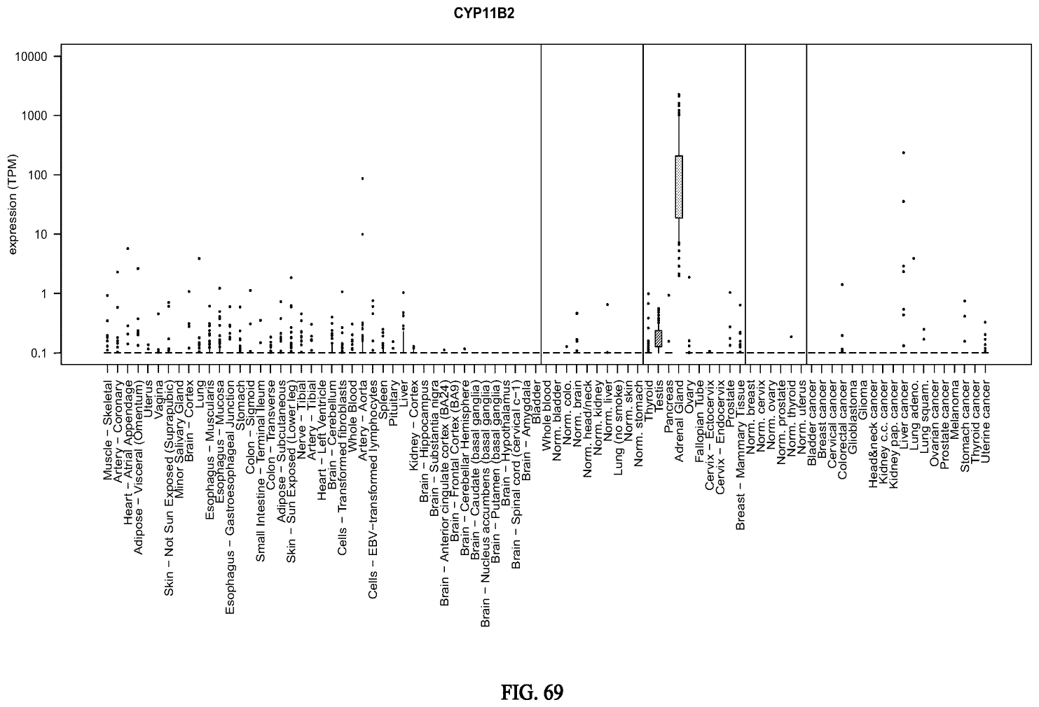

FIG. 70 is a boxplot illustrating expression levels of gene STAR in a number of different normal tissues and tumors.

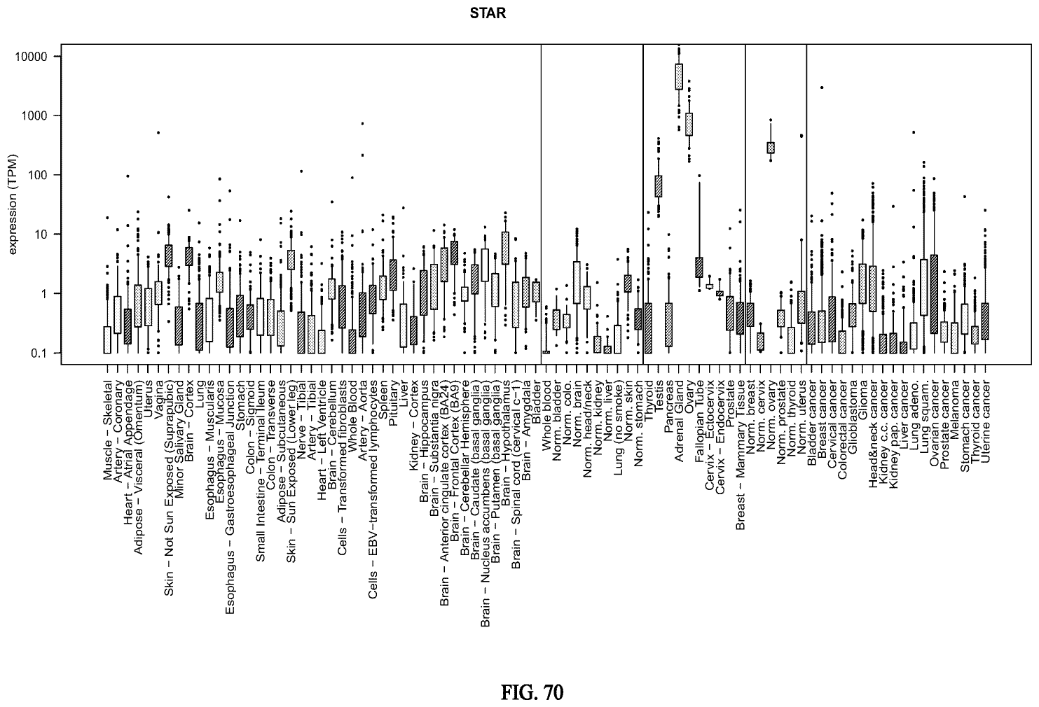

FIG. 71 is a boxplot illustrating expression levels of gene CYP11A1 in a number of different normal tissues and tumors.

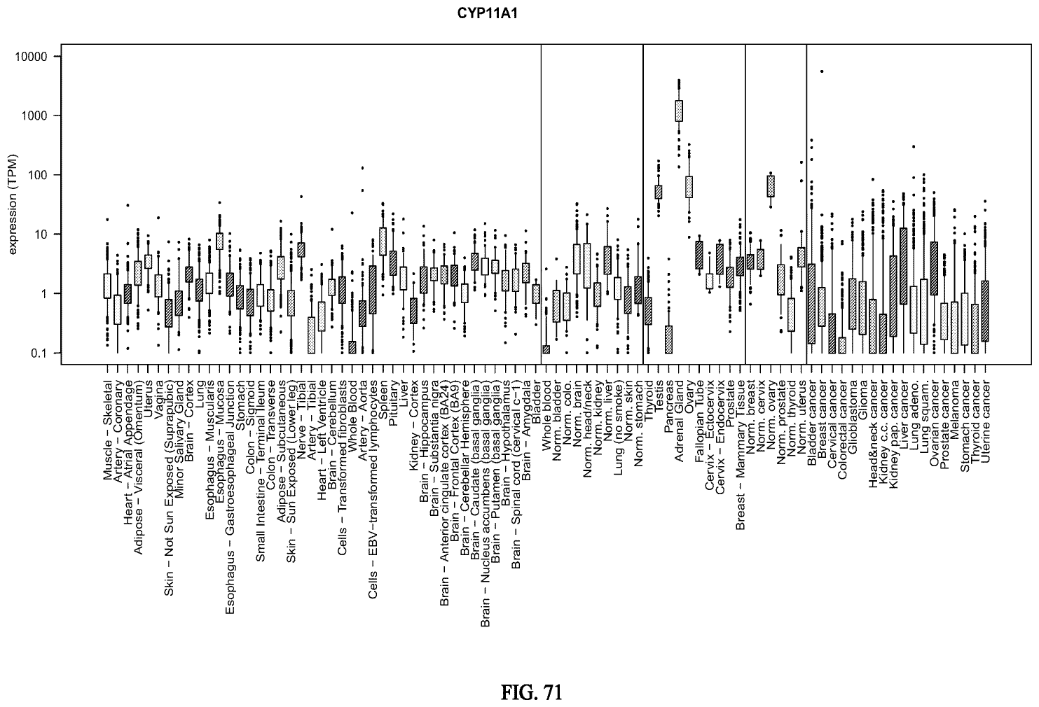

FIG. 72 is a boxplot illustrating expression levels of gene MC2R in a number of different normal tissues and tumors.

FIG. 73 shows a schematic of an exemplary workflow for epitope mapping using targeted proteomics. FIG. 73 discloses SEQ ID NO: 8970.

FIG. 74 depicts exemplary, graphs demonstrating spectral validation of class I HLA epitopes by mass spectrometry of endogenous peptides using targeted proteomics. Chromatographs of 6 characteristic fragment ions for the light (endogenous) (“HPEYNRPLL” disclosed as SEQ ID NO: 4143) and heavy isotope-labeled synthetic peptide sequence “HPEYNRPLL (SEQ ID NO: 8969)” derived from KLK4 (HLA*B-07:02, where the endogenous peptide was identified in a human prostate specimen) are shown. Matched chromatography retention times and a high dot product similarity score (0.992, calculated using Skyline software) of peptide fragment ions provide validation that this epitope is processed and presented on the HLA-B*07:02 molecule.

FIG. 75 depicts two exemplary spectrums showing spectral validation of endogenous peptides using targeted proteomics. Spectrum for the light (endogenous) HPEYNRPLL (SEQ ID NO: 4143) epitope identified on a human prostate specimen (left) and the corresponding heavy isotope-labeled synthetic peptide (right) (“HPEYNRPLL” disclosed as SEQ ID NO: 8969) are shown. B and Y fragment ions are shown, and display high spectral similarity, confirming detection of the endogenous epitope. For each peptide, the top 200 more intense ions were plotted, and corresponding mass error of highlighted b and y ions are plotted below spectrum plots.

FIG. 76 depicts exemplary flow cytometry plots of peptide-MHC multimer staining of target epitopes after naive T cell inductions in healthy donors with the indicated HLA-I molecules. Multimer positive populations and the percentage of multimer positive cells is shown. The top panel displays positive sample identifications using a combinatorial multimer analysis. The bottom panel displays results from a confirmation combinatorial analysis performed on frozen samples following the initial identification from the top panel. Multimer positive cells from analyses in the bottom panel are sorted for downstream TCR identification. FIG. 76 discloses SEQ ID NOS 4143, 3757 and 6095, respectively, in order of appearance.

FIG. 77 depicts graphs showing exemplary TCR clonotypes identified from the 10× genomics pipeline. Each graph originated from a single sorted, multimer positive, population. The samples in this case all contained two unique TCR clonotypes, identified by a paired alpha and beta sequence. In the case where the 10× genomics pipeline identified a clonotype that contained multiple of either alpha or beta sequences, all possible combinations were synthesized for antigen specificity and avidity.

FIG. 78 depicts exemplary plots showing avidity of exemplary TCRs. The plots reflect the CD69 expression on transduced Jurkat cells (identified by the co-expression of murine TCR, CD8, and CD3) following an overnight coculture with a target cell line presenting the HLA and loaded with a variable amount of peptide. Of the seven TCRs tested, five of them show increased expression of CD69 in a peptide dependent manner. The concentration required to achieve a 50% activation (EC50) is calculated from these plots and the results are shown on the plot. Target cells were previously transduced to overexpress the allele of interest. A375s were plated at 50K/well 2-5 hours prior to pulsing with peptide for 1 hour prior to the addition of effector cells. T2s were plated at 10K/well prior to pulsing with peptide for 1 hour prior to the addition of effector cells. Peptide was pulsed with a final concentration between 10e3 and 10e-1 nM. The cells were co-cultured overnight before harvest and staining for CD69 expression via flow using a CD8, CD3, and murine TCR constant antibodies as lineage markers for effector cells.

FIG. 79 depicts exemplary plots showing endogenous activity of two different exemplary TcRs. avidity of exemplary TcRs, the plots here reflect the activation of two different TCR sequences (hereafter named mTCR21-033 and mTCR-034) following a coculture with the cell line mDA-PCa-2b which is endogenous for both HLA-B07 and KLK4. These plots are showing an increase in activation of mTCR21-033, but not mTCR21-034 following a 24-hour treatment with a cocktail of interferons (IFN). The IFN treatment increases the expression of surface HLA on cell lines, and the increased surface expression of the HLA can provide more expression of HLA-B07 bound to the KLK4 epitope, mDA-PCa-2b cells were plated at 50K/well in F12K media. The next day the cultures were treated with a cocktail of interferon alpha, beta, and gamma all at 1 U/μL final concentration. The next day the cells were washed with RPMI supplemented with 10% FBS and Glutamax. The cultures were then pulsed with peptide at a final concentration of 2 μM for 1 hour before the addition of effector cells. The cells were co-cultured overnight before harvest and staining for CD69 expression via flow using a CD8, CD3, and murine TCR constant antibodies as lineage markers for effector cells and HLA-B07 as a lineage marker for the target cells.

### DETAILED DESCRIPTION

### Definitions

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually: C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example. “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” can mean that a particular feature, structure, or characteristic described in connection with the embodiments is included in at least some embodiments, but not necessarily all embodiments, of the present disclosures. To facilitate an understanding of the present disclosure, a number of terms and phrases are defined below.

“Major Histocompatibility Complex” or “MHC” can refer to a cluster of genes that plays a role in control of the cellular interactions responsible for physiologic immune responses. In humans, the MHC complex is also known as the human leukocyte antigen (HLA) complex. For a detailed description of the MHC and HLA complexes, see, Paul, Fundamental Immunology, 3^{rd} Ed., Raven Press, New York (1993). “Proteins or molecules of the major histocompatibility complex (MHC)”, “MHC molecules”, “MHC proteins” or “HLA proteins” are to be understood as meaning proteins capable of binding peptides resulting from the proteolytic cleavage of protein antigens transporting them to the cell surface and presenting them there to specific cells, in particular cytotoxic T-lymphocytes, T-helper cells, or B cells. The major histocompatibility complex in the genome comprises the genetic region whose gene products expressed on the cell surface are important for binding and presenting endogenous and/or foreign antigens and thus for regulating immunological processes. The major histocompatibility complex is classified into two gene groups coding for different proteins, namely molecules of MHC class I and molecules of MHC class II. The cellular biology and the expression patterns of the two MHC classes are adapted to these different roles.

“Human Leukocyte Antigen” or “HLA” can refer to a human class I or class II Major Histocompatibility Complex (MHC) protein (see, e.g., Stites, et al., Immunology, 8^{th} Ed., Lange Publishing, Los Altos, Calif. (1994).

“Polypeptide” and “peptide” are used interchangeably and as used herein can refer to a polymer of amino acid residues. A “mature protein” is a protein which is full-length and which, optionally, includes glycosylation or other modifications typical for the protein in a given cellular environment. Polypeptides and proteins disclosed herein (including functional portions and functional variants thereof) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine. The present disclosure further contemplates that expression of polypeptides described herein in an engineered cell can be associated with post-translational modifications of one or more amino acids of the polypeptide constructs. Non-limiting examples of post-translational modifications include phosphorylation, acylation including acetylation and formylation, glycosylation (including N-linked and O-linked), amidation, hydroxylation, alkylation including methylation and ethylation, ubiquitination, addition of pyrrolidone carboxylic acid, formation of disulfide bridges, sulfation, myristoylation, palmitoylation, isoprenylation, farnesylation, geranylation, glypiation, lipoylation and iodination. The nomenclature used to describe peptides or proteins follows the conventional practice wherein the amino group is presented to the left (the amino- or N-terminus) and the carboxyl group to the right (the carboxy- or C-terminus) of each amino acid residue. When amino acid residue positions are referred to in a peptide epitope they are numbered in an amino to carboxyl direction with position one being the residue located at the amino terminal end of the epitope, or the peptide or protein of which it can be a part. In the formula representing selected specific embodiments of the present disclosure, the amino- and carboxyl-terminal groups, although not specifically shown, are in the form they would assume at physiologic pH values, unless otherwise specified. In the amino acid structure formula, each residue is generally represented by standard three letter or single letter designations. The L-form of an amino acid residue is represented by a capital single letter or a capital first letter of a three-letter symbol, and the D-form for those amino acid residues having D-forms is represented by a lower case single letter or a lower case three letter symbol. However, when three letter symbols or full names are used without capitals, they can refer to L amino acid residues. Glycine has no asymmetric carbon atom and is simply referred to as “Gly” or “G”. The amino acid sequences of peptides set forth herein are generally designated using the standard single letter symbol. (A, Alanine; C. Cysteine; D, Aspartic Acid; E, Glutamic Acid; F, Phenylalanine; G, Glycine; H, Histidine: I, Isoleucine; K, Lysine: L, Leucine; M, Methionine; N, Asparagine; P, Proline; Q, Glutamine; R, Arginine; S, Serine: T, Threonine: V, Valine; W. Tryptophan; and Y, Tyrosine.)

An “immunogenic” peptide or an “immunogenic” epitope can refer to a peptide or a peptide containing an epitope that comprises an allele-specific motif such that the peptide will bind an HLA molecule and induce a cell-mediated or humoral response, for example, cytotoxic T lymphocyte (CTL (e.g., CD8+)), helper T lymphocyte (Th (e.g., CD4*)) and/or B lymphocyte response. Thus, immunogenic peptides described herein are capable of binding to an appropriate HLA molecule and thereafter inducing a CTL (cytotoxic) response, or a HTL (and humoral) response, to the peptide.

A “reference” can be used to correlate and compare the results obtained in the methods of the present disclosure from a tumor specimen. Typically the “reference” may be obtained on the basis of one or more normal specimens, in particular specimens which are not affected by a cancer disease, either obtained from a patient or one or more different individuals, for example, healthy individuals, in particular individuals of the same species. A “reference” can be determined empirically by testing a sufficiently large number of normal specimens.

An “epitope” can be the collective features of a molecule, such as primary, secondary and tertiary peptide structure, and charge, that together form a site recognized by, for example, an immunoglobulin, T cell receptor, HLA molecule, or chimeric antigen receptor. Alternatively, an epitope can be defined as a set of amino acid residues which is involved in recognition by a particular immunoglobulin, or in the context of T cells, those residues necessary for recognition by T cell receptor proteins, chimeric antigen receptors, and/or Major Histocompatibility Complex (MHC) receptors. Epitopes can be prepared by isolation from a natural source, or they can be synthesized according to standard protocols in the art. Synthetic epitopes can comprise artificial amino acid residues, “amino acid mimetics,” such as D isomers of naturally-occurring L amino acid residues or non-naturally-occurring amino acid residues such as cyclohexylalanine. Throughout this disclosure, epitopes may be referred to in some cases as peptides or peptide epitopes. It is to be appreciated that proteins or peptides that comprise an epitope or an analog described herein as well as additional amino acid(s) are still within the bounds of the present disclosure. In certain embodiments, the peptide comprises a fragment of an antigen. In certain embodiments, there is a limitation on the length of a peptide of the present disclosure. The embodiment that is length-limited occurs when the protein or peptide comprising an epitope described herein comprises a region (i.e., a contiguous series of amino acid residues) having 100% identity with a native sequence. In order to avoid the definition of epitope from reading, e.g., on whole natural molecules, there is a limitation on the length of any region that has 100% identity with a native peptide sequence. Thus, for a peptide comprising an epitope described herein and a region with 100% identity with a native peptide sequence, the region with 100% identity to a native sequence generally has a length of: less than or equal to 600 amino acid residues, less than or equal to 500 amino acid residues, less than or equal to 400 amino acid residues, less than or equal to 250 amino acid residues, less than or equal to 100 amino acid residues, less than or equal to 85 amino acid residues, less than or equal to 75 amino acid residues, less than or equal to 65 amino acid residues, and less than or equal to 50 amino acid residues. In certain embodiments, an “epitope” described herein is comprised by a peptide having a region with less than 51 amino acid residues that has 100% identity to a native peptide sequence, in any increment down to 5 amino acid residues; for example 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 amino acid residues.

A “T cell epitope” is to be understood as meaning a peptide sequence which can be bound by the MHC molecules of class I or II in the form of a peptide-presenting MHC molecule or MHC complex and then, in this form, be recognized and bound by T cells, such as T-lymphocytes or T-helper cells.

As used herein, the term “affinity” can refer to a measure of the strength of binding between two members of a binding pair, for example, an HLA-binding peptide and a class I or II HLA. Kr is the dissociation constant and has units of molarity. The affinity constant is the inverse of the dissociation constant. An affinity constant is sometimes used as a generic term to describe this chemical entity. It is a direct measure of the energy of binding. Affinity may be determined experimentally, for example by surface plasmon resonance (SPR) using commercially available Biacore SPR units. Affinity may also be expressed as the inhibitory concentration 50 (IC₅₀), that concentration at which 50% of the peptide is displaced. Likewise, In(IC₅₀) refers to the natural log of the IC₅₀. K_{off} refers to the off-rate constant, for example, for dissociation of an HLA-binding peptide and a class I or 11 HLA. Throughout this disclosure, “binding data” results can be expressed in terms of “IC₅₀.” IC₅₀ is the concentration of the tested peptide in a binding assay at which 50% inhibition of binding of a labeled reference peptide is observed. Given the conditions in which the assays are run (i.e., limiting HLA protein and labeled reference peptide concentrations), these values approximate K_{D} values. Assays for determining binding are well known in the art and are described in detail, for example, in PCT publications WO 94/20127 and WO 94/03205, and other publications such Sidney et al., Current Protocols in Immunology 18.3.1 (1998); Sidney, et al., J. Immunol. 154:247 (1995): and Sette, et al., Mol. Immunol. 31:813 (1994). Alternatively, binding can be expressed relative to binding by a reference standard peptide. For example, can be based on its IC₅₀, relative to the IC % of a reference standard peptide. Binding can also be determined using other assay systems including those using: live cells (e.g., Ceppellini et al., Nature 339:392 (1989); Christnick et al., Nature 352:67 (1991); Busch et al., Int. Immunol. 2:443 (1990); Hill et al., J. Immunol. 147:189 (1991); del Guercio et al., J. Immunol. 154:685 (1995)), cell free systems using detergent lysates (e.g., Cerundolo et al., J. Immunol. 21:2069 (1991)), immobilized purified MHC (e.g., Hill et al., J. Immunol. 152, 2890 (1994); Marshall et al., J. Immunol. 152:4946 (1994)), ELISA systems (e.g., Reay et al., EMBO J. 11:2829 (1992)), surface plasmon resonance (e.g., Khilko et al., J. Biol. Chem. 268:15425 (1993)): high flux soluble phase assays (Hammer et al., J. Exp. Med. 180:2353 (1994)), and measurement of class I MHC stabilization or assembly (e.g., Ljunggren et al., Nature 346:476 (1990); Schumacher et al., Cell 62:563 (1990); Townsend et al., Cell 62:285 (1990); Parker et al., J. Immunol. 149:1896 (1992)). “Cross-reactive binding” indicates that a peptide is bound by more than one HLA molecule: a synonym is degenerate binding.

“Synthetic peptide” can refer to a peptide that is obtained from a non-natural source, e.g., is man-made. Such peptides can be produced using such methods as chemical synthesis or recombinant DNA technology. In some embodiments, “Synthetic peptides” may include “fusion proteins.”

The term “motif” can refer to a pattern of residues in an amino acid sequence of defined length, for example, a peptide of less than about 15 amino acid residues in length, or less than about 13 amino acid residues in length, for example, from about 8 to about 13 amino acid residues (e.g., 8, 9, 10, 11, 12, or 13) for a class I HLA motif and from about 6 to about 25 amino acid residues (e.g., 6, 7, 8, 9, 10, 11, 12, 13, 14, 15, 16, 17, 18, 19, 20, 21, 22, 23, 24, or 25) for a class II HLA motif, which is recognized by a particular HLA molecule. Motifs are typically different for each HLA protein encoded by a given human HLA allele. These motifs differ in their pattern of the primary and secondary anchor residues. In some embodiments, an MHC class I motif identifies a peptide of 9, 10, or 11 amino acid residues in length.

According to the present disclosure, the term “vaccine” can relate to a pharmaceutical preparation (pharmaceutical composition) or product that upon administration induces an immune response, for example, a cellular or humoral immune response, which recognizes and attacks a pathogen or a diseased cell such as a cancer cell. A vaccine may be used for the prevention or treatment of a disease. The term “individualized cancer vaccine” or “personalized cancer vaccine” concerns a particular cancer patient and means that a cancer vaccine is adapted to the needs or special circumstances of an individual cancer patient.

A“protective immune response” or “therapeutic immune response” can refer to a CTL and/or an HTL response to an antigen derived from an pathogenic antigen (e.g., a tissue-specific antigen), which in some way prevents or at least partially arrests disease symptoms, side effects or progression. The immune response can also include an antibody response which has been facilitated by the stimulation of helper T cells.

The term “antibody” as used herein, can refer to an immunoglobulin protein comprising two heavy chains, bound to each other, wherein each heavy chain can also be paired with a light chain.

A “functional part of an antibody” as used herein can refer to a part that has at least one shared property as said antibody in kind, not necessarily in amount. The functional part is capable of binding the same antigen as the antibody, albeit not necessarily to the same extent. A functional part of an antibody preferably comprises at least a heavy chain variable domain (Vi) and a light chain variable domain (V). In some embodiments, a functional part of an antibody comprises at least a heavy chain variable domain (V_{H}). Non-limiting examples of a functional part of an antibody can be a single domain antibody, a single chain antibody, a nanobody, an unibody, a single chain variable fragment (scFv), a bi-specific T-cell engager (BiTE), a Fab fragment and a F(ab′)₂ fragment.

“Antigen processing” or “processing” and its grammatical equivalents can refer to the degradation of a polypeptide or antigen into procession products, which are fragments of said polypeptide or antigen (e.g., the degradation of a polypeptide into peptides) and the association of one or more of these fragments (e.g., via binding) with MHC molecules for presentation by cells, for example, antigen presenting cells, to specific T cells.

“Antigen presenting cells” (APC) can be cells which present peptide fragments of protein antigens in association with MHC molecules on their cell surface. Some APCs may activate antigen specific T cells. Professional antigen-presenting cells are very efficient at internalizing antigen, either by phagocytosis or by receptor-mediated endocytosis, and then displaying a fragment of the antigen, bound to a class II MHC molecule, on their membrane. The T cell recognizes and interacts with the antigen-class II MHC molecule complex on the membrane of the antigen presenting cell. An additional co-stimulatory signal is then produced by the antigen presenting cell, leading to activation of the T cell. The expression of co-stimulatory molecules is a defining feature of professional antigen-presenting cells. The main types of professional antigen-presenting cells are dendritic cells, which have the broadest range of antigen presentation, and are probably the most important antigen presenting cells, macrophages, B-cells, and certain activated epithelial cells. Dendritic cells (DCs) are leukocyte populations that present antigens captured in peripheral tissues to T cells via both MHC class II and I antigen presentation pathways. It is well known that dendritic cells are potent inducers of immune responses and the activation of these cells is a critical step for the induction of antitumoral immunity. Dendritic cells are conveniently categorized as “immature” and “mature” cells, which can be used as a simple way to discriminate between two well characterized phenotypes. However, this nomenclature should not be construed to exclude all possible intermediate stages of differentiation. Immature dendritic cells are characterized as antigen presenting cells with a high capacity for antigen uptake and processing, which correlates with the high expression of Fc receptor (FcR) and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1 BB).

The terms “identical” and its grammatical equivalents as used herein or “sequence identity” in the context of two nucleic acid sequences or amino acid sequences of polypeptides can refer to the residues in the two sequences which are the same when aligned for maximum correspondence over a specified comparison window. A “comparison window”, as used herein, can refer to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman, Adv. Appl. Math., 2:482 (1981); by the alignment algorithm of Needleman and Wunsch, J. Mol. Biol., 48:443 (1970); by the search for similarity method of Pearson and Lipman, Proc. Nat. Acad. Sci. U.S.A., 85:2444 (1988); by computerized implementations of these algorithms (including, but not limited to CLUSTAL in the PC/Gene program by Intelligentics, Mountain View Calif., GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp, Gene, 73:237-244 (1988) and Higgins and Sharp, CABIOS, 5:151-153 (1989); Corpet et al., Nucleic Acids Res., 16:10881-10890 (1988); Huang et al., Computer Applications in the Biosciences, 8:155-165 (1992); and Pearson et al., Methods in Molecular Biology, 24:307-331 (1994). Alignment is also often performed by inspection and manual alignment. In one class of embodiments, the polypeptides herein have at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference polypeptide, or a fragment thereof, e.g., as measured by BLASTP (or CLUSTAL, or any other available alignment software) using default parameters. Similarly, nucleic acids can also be described with reference to a starting nucleic acid, e.g., they can have 50%, 60%, 70%, 75%, 80%, 85%, 90%, 98%, 99% or 100% sequence identity to a reference nucleic acid or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, said percentage of residues in the smaller molecule finds a match residue in the larger molecule in accordance with the order by which the two molecules are optimally aligned.

The term “substantially identical” and its grammatical equivalents as applied to nucleic acid or amino acid sequences can mean that a nucleic acid or amino acid sequence comprises a sequence that has at least 90% sequence identity or more, at least 95%, at least 98% and at least 99%, compared to a reference sequence using the programs described above, e.g., BLAST, using standard parameters. For example, the BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1992)). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. In embodiments, the substantial identity exists over a region of the sequences that is at least about 50 residues in length, over a region of at least about 100 residues, and in embodiments, the sequences are substantially identical over at least about 150 residues. In embodiments, the sequences are substantially identical over the entire length of the coding regions.

The term “vector” as used herein can mean a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

A polypeptide, antibody, polynucleotide, vector, cell, or composition which is “isolated” can be a polypeptide, antibody, polynucleotide, vector, cell, or composition which is in a form not found in nature. Isolated polypeptides, antibodies, polynucleotides, vectors, cells, or compositions include those which have been purified to a degree that they are no longer in a form in which they are found in nature. In some embodiments, a polypeptide, antibody, polynucleotide, vector, cell, or composition which is isolated is substantially pure. For example, isolated peptides do not contain some or all of the materials normally associated with the peptides in their in situ environment. For example, a naturally-occurring polynucleotide or peptide present in a living animal is not isolated, but the same polynucleotide or peptide, separated from some or all of the coexisting materials in the natural system, is isolated. Such a polynucleotide could be part of a vector, and/or such a polynucleotide or peptide could be part of a composition, and still be “isolated” in that such vector or composition is not part of its natural environment. Isolated RNA molecules include in vivo or in vitro RNA transcripts of the DNA molecules described herein, and further include such molecules produced synthetically.

The terms “polynucleotide”, “nucleotide”, “nucleic acid”, “polynucleic acid” or “oligonucleotide” and their grammatical equivalents arc used interchangeably herein and can refer to polymers of nucleotides of any length, and include DNA and RNA, for example, mRNA. Thus, these terms includes double and single stranded DNA, triplex DNA, as well as double and single stranded RNA. It also includes modified, for example, by methylation and/or by capping, and unmodified forms of the polynucleotide. The term is also meant to include molecules that include non-naturally occurring or synthetic nucleotides as well as nucleotide analogs. The nucleic acid sequences and vectors disclosed or contemplated herein may be introduced into a cell by, for example, transfection, transformation, or transduction. The nucleotides can be deoxyribonucleotides, ribonucleotides, modified nucleotides or bases, and/or their analogs, or any substrate that can be incorporated into a polymer by DNA or RNA polymerase. In some embodiments, the polynucleotide and nucleic acid can be in vitro transcribed mRNA. In some embodiments, the polynucleotide that is administered using the methods of the present disclosure is mRNA.

“Transfection,” “transformation,” or “transduction” as used herein can refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.). Methods in Molecular Biology, Vol. 7, Gene Transfer and Expression Protocols, Humana Press (1991)); DEAE-dextran: electroporation; cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)). Phage or viral vectors can be introduced into host cells, after growth of infectious particles in suitable packaging cells, many of which are commercially available.

The term “subject” can refer to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The terms “effective amount” or “therapeutically effective amount” or “therapeutic effect” can refer to an amount of a therapeutic effective to “treat” a disease or disorder in a subject or mammal. The therapeutically effective amount of a drug has a therapeutic effect and as such can prevent the development of a disease or disorder; slow down the development of a disease or disorder; slow down the progression of a disease or disorder; relieve to some extent one or more of the symptoms associated with a disease or disorder; reduce morbidity and mortality; improve quality of life; or a combination of such effects.

The terms “treating” or “treatment” or “to treat” or “alleviating” or “to alleviate” can refer to both (1) therapeutic measures that cure, slow down, lessen symptoms of, and/or halt progression of a diagnosed pathologic condition or disorder; and (2) prophylactic or preventative measures that prevent or slow the development of a targeted pathologic condition or disorder. Thus those in need of treatment include those already with the disorder; those prone to have the disorder, and those in whom the disorder is to be prevented.

“Pharmaceutically acceptable” can refer to a generally non-toxic, inert, and/or physiologically compatible composition or component of a composition.

A “pharmaceutical excipient” or “excipient” can comprise a material such as an adjuvant, a carrier, pH-adjusting and buffering agents, tonicity adjusting agents, wetting agents, preservatives, and the like. A “pharmaceutical excipient” is an excipient which is pharmaceutically acceptable.

A “tissue-specific” antigen can refer to an epitope sequence that is encoded by a gene that has a higher expression level in a target tissue than a non-target tissue.

### Tissue-Specific Antigens

Tissue-specific antigens can have great potential as targets for immunotherapies. Among others, provided herein are tissue-specific antigens, compositions containing tissue-specific antigens or producing tissue-specific antigens, and methods of identifying tissue-specific antigens. One advantage of targeting tissue-specific antigens for immunotherapies can be that tissue-specific genes are typically expressed very highly in their given tissue, enhancing their likelihood of robust presentation. It is possible such an approach can eliminate both the tumor as well as the corresponding healthy tissue of the same lineage. However, in many cases, this can be an acceptable trade-off. For instance, CAR-T therapies targeting the CD19 surface marker eliminate both healthy B cells and leukemic B cells. While the loss of normal B cells may compromise immune function, patients are able to tolerate B cell ablation.

In some embodiments, the tissue-specific antigens are specific to non-essential tissues. The tissue-specific epitope sequence can be expected to be presented on tumor cells or non-essential normal cells from a non-essential tissue of the same lineage, and can be expected to have zero or a low expression level in essential tissues. The epitope sequence information of the tissue-specific antigens, e.g., antigens specific to a tumor from a particular tissue, can therefore be translated into therapeutic methods and compositions for diseases or conditions, e.g., cancer. In some embodiments, the tissue-specific antigens provided herein can be expressed at a high level in a tumor tissue that originates or is at a non-essential tissue. The tissue-specific antigens, in some embodiments, may or may not be expressed in a normal non-essential tissue, while they can be expressed at a relatively very low level in essential tissues.

As provided herein, a tissue-specific antigen can refer to an epitope sequence that is encoded by a gene that has a higher expression level in a target tissue than a non-target tissue, in which case, the tissue-specific antigen can be referred to as being “specific to the target tissue”. In some embodiments, a target tissue-specific antigen is from an epitope gene that has an expression level in the target tissue that is at least 1.1, at least 1.2, at least 1.3, at least 1.4, at least 1.5, at least 1.6, at least 1.7, at least 1.8, at least 1.9, at least 2, at least 2.1, at least 2.3, at least 2.4, at least 2.5, at least 2.6, at least 2.7, at least 2.8, at least 2.9, at least 3, at least 3.2, at least 3.4, at least 3.5, at least 3.6, at least 3.8, at least 4, at least 4.5, at least 5, at least 6, at least 7, at least 8, at least 9, at least 10, at least 11, at least 12, at least 13, at least 14, at least 15, at least 16, at least 17, at least 18, at least 19, at least 20, at least 22, at least 24, at least 25, at least 26, at least 28, at least 30, at least 35, at least 40, at least 45, at least 50, at least 55, at least 60, at least 70, at least 80, at least 90, at least 100, at least 120, at least 140, at least 150, at least 160, at least 180, at least 200, at least 250, at least 300, at least 400, at least 500, at least 600, at least 700, at least 800, at least 900, at least 1000, at least 2000, at least 4000, at least 5000, at least 10⁴, at least 10⁵, or at least 10⁶ folds higher than its expression level in a second tissue. In some embodiments, the tissue-specific antigen can be specific to one certain type of tissue, for example, the tissue-specific antigen can be only specific to pancreatic tissue, heart tissue, prostate tissue, or epithelial tissue. In some embodiments, the tissue-specific antigen can be specific to more than one type of tissues, for example, the tissue-specific antigen can be specific to 2, 3, 4, 5, 6, 7, 8, 9, 10, 11, 12, 13, 14, 15, or more different types of tissues. The criteria for setting “tissue specificity” can vary depending on purposes of application of the subject matter provided herein. As will be discussed in details, the subject matter provided herein can be applied to various situations where different criteria for selecting tissue-specific antigens may be utilized.

In some embodiments, the tissue-specific antigen is specific to a target tissue other than in an essential tissue. In some embodiments, the target tissue is a non-essential tissue. As provided herein, an essential tissue can refer to a tissue in a living body, whose function in the maintaining the life of the body cannot be substituted by an internal or external support. As provided herein, a non-essential tissue can refer to a tissue in a living body, whose function in the maintaining the life of the body can be substituted (e.g., function of the tissue can be at least partially performed by some other tissue in the body or performed by tissue transplant or an artificial device) or foregone (e.g., function of the tissue is not required for survival of the body). In some embodiments, an essential tissue comprises brain or colon tissue. In some embodiments, an essential tissue comprises bone marrow. In some embodiments, a non-essential tissue comprises thyroid, pancreas, adrenal, fallopian, prostate, breast, ovary, or cervical tissue.

In some aspects, the present disclosure provides tissue-specific antigens, e.g. tissue-specific antigenic peptides. The tissue-specific antigens provided herein can comprise tumor epitope sequences. The tissue-specific antigens as provided herein can comprise tumor epitope sequences from tumor expressed proteins as provided herein. In some embodiments, a tumor expressed protein as provided herein is specific to a tumor from a certain type of tissue, for example, tumor expressed protein TSHR can be specific to thyroid cancer that is from thyroid tissue.

In some embodiments, the tumor expressed proteins as provided herein comprise ACTL7A, ACTL7B, ACTL9, ACTRT2, ADAD1, AKAP4, ALPPL2, AMY2A, ANKRD30A, AQP12A, AQP12B, C2orf53, CCDC70, CELA2A, CELA2B, CETN1, CLDN6, COL10A1, CSAG1, CTCFL, CTRC, CYP11A1, CYP11B1, CYP11B2, DCAF4L2, DLL3, DMRTB1, EPYC, G6PC2, HMGB4, IAPP, KIF2B, KIRREL2, KLK2, KLK3, KLK4, LELP1, MAGEA11, MAGEA12, MAGEA2, MAGEA4, MAGEC2, MC2R, MMP13, PAGE5, PGK2, PNLIPRP1, POTEE, POTEG, POTEH, PPIAL4G, PRAME, PRDM7, PRM1, PRM2, RBPJL, RLN1, RSPH6A, SCXB, SERPINI2, SLC45A2, SPATA8, SSX1, STAR, SYCN, TG, TGM4, TNP1, TPD52L3, TSHR, TSPAN10, UBQLN3, or any combination thereof.

The tumor expressed proteins provided herein can comprise TSHR, TG, RSPH6A, SCXB, SSX1, or any combination thereof, each of which can be specific to thyroid cancer. The epitope sequence provided herein can have at least 50%, at least 60%, at least 70%, at least 80%, at least 85%, at least 90%, at least 95%, or 100% sequence identity to peptide sequence selected from the group consisting of SEQ ID NOS 6846-7061, 7359-7448, 7629-8099, and 8619-8744, each of which can be specific to thyroid cancer. The epitope sequence provided herein can have at least 70% sequence identity to peptide sequence selected from the group consisting of SEQ ID NOS 6846-7061, 7359-7448, 7629-8099, and 8619-8744, each of which can be specific to thyroid cancer.

The tumor expressed proteins provided herein can comprise RBPJL, AQP12A, AQP12B, IAPP, CELA2A, CELA2B, AMY2A, CTRC, G6PC2, KIRREL2, PNLIPRP1, SERPINI2, SYNC, or any combination thereof, each of which can be specific to pancreatic cancer. The epitope sequence provided herein can have at least 50%, at least 60%, at least 70%, at least 80%, at least 85%, at least 90%, at least 95%, or 100% sequence identity to peptide sequence selected from the group consisting of SEQ ID NOS 720-814, 989-1182, 1373-1565, 2120-2211, 2920-3009, 3101-3196, 3320-3440, 5193-5284, 6487-6579, 7062-7150, and 7539-7628, each of which can be specific to pancreatic cancer. The epitope sequence provided herein can have at least 70% sequence identity to peptide sequence selected from the group consisting of SEQ ID NOS 720-814, 989-1182, 1373-1565, 2120-2211, 2920-3009, 3101-3196, 3320-3440, 5193-5284, 6487-6579, 7062-7150, and 7539-7628, each of which can be specific to pancreatic cancer.

The tumor expressed proteins provided herein can comprise CYP11A1, CYP11B1, CYP11B2, MC2R, STAR, or any combination thereof, each of which can be specific to adrenal cancer. The epitope sequence provided herein can have at least 50%, at least 60%, at least 70%, at least 80%, at least 85%, at least 90%, at least 95%, or 100/6 sequence identity to peptide sequence selected from the group consisting of SEQ ID NOS 2212-2523, 4817-4915, and 7449-7538, each of which can be specific to adrenal cancer. The epitope sequence provided herein can have at least 70% sequence identity to peptide sequence selected from the group consisting of SEQ ID NOS 2212-2523, 4817-4915, and 7449-7538, each of which can be specific to adrenal cancer.

The tumor expressed proteins provided herein can comprise ALPPL2, POTEE, PRAME, or any combination thereof, each of which can be specific to uterine cancer. The epitope sequence provided herein can have at least 50%, at least 60%, at least 70%, at least 80%, at least 85%, at least 90%, at least 95%, or 100% sequence identity to peptide sequence selected from the group consisting of SEQ ID NOS 627-719, 5285-5431, and 6085-6183, each of which can be specific to uterine cancer. The epitope sequence provided herein can have at least 70% sequence identity to peptide sequence selected from the group consisting of SEQ ID NOS 627-719, 5285-5431, and 6085-6183, each of which can be specific to uterine cancer.

The tumor expressed proteins provided herein can comprise KLK2, KLK3, KLK4, POTEH, POTEG, TGM4, RLN1, POTEE, PPIAL4G or any combination thereof, each of which can be specific to prostate cancer. The epitope sequence provided herein can have at least 50%, at least 60%, at least 70%, at least 80%, at least 85%, at least 90%, at least 95%, or 100% sequence identity to peptide sequence selected from the group consisting of SEQ ID NOS 3441-4274, 5285-6084, 6580-6845, and 8100-8434, each of which can be specific to prostate cancer. The epitope sequence provided herein can have at least 70% sequence identity to peptide sequence selected from the group consisting of SEQ ID NOS 3441-4274, 5285-6084, 6580-6845, and 8100-8434, each of which can be specific to prostate cancer.

The tumor expressed proteins provided herein can comprise ANKRD30A, COL10A1, or a combination, each of which can be specific to breast cancer. The epitope sequence provided herein can have at least 50%, at least 60%, at least 70%, at least 80%, at least 85%, at least 90%, at least 95%, or 100% sequence identity to peptide sequence selected from the group consisting of SEQ ID NOS 815-988, and 1749-1867, each of which can be specific to breast cancer. The epitope sequence provided herein can have at least 70% sequence identity to peptide sequence selected from the group consisting of SEQ ID NOS 815-988, and 1749-1867, each of which can be specific to breast cancer.

The tumor expressed proteins provided herein can comprise CTCFL, PRAME, CLDN6, EPYC, or any combination thereof, each of which can be specific to ovarian cancer. The epitope sequence provided herein can have at least 50%, at least 60%, at least 70%, at least 80%, at least 85%, at least 90%, at least 95%, or 100% sequence identity to peptide sequence selected from the group consisting of SEQ ID NOS 1659-1748, 1964-2119, 2827-2919, and 6085-6183, each of which can be specific to ovarian cancer. The epitope sequence provided herein can have at least 70% sequence identity to peptide sequence selected from the group consisting of SEQ ID NOS 1659-1748, 1964-2119, 2827-2919, and 6085-6183, each of which can be specific to ovarian cancer.

The tumor expressed proteins provided herein can comprise CTCFL, each of which can be specific to cervical cancer. The epitope sequence provided herein can have at least 50%, at least 60%, at least 70%, at least 80%, at least 85%, at least 90%, at least 95%, or 100% sequence identity to peptide sequence selected from the group consisting of SEQ ID NOS 1964-2119, each of which can be specific to cervical cancer. The epitope sequence provided herein can have at least 70% sequence identity to peptide sequence selected from the group consisting of SEQ ID NOS 1964-2119, each of which can be specific to cervical cancer.

The tumor expressed proteins provided herein can comprise POTEE, PPIAL4G, or a combination thereof, each of which can be specific to colorectal cancer. The epitope sequence provided herein can have at least 50%, at least 60%, at least 70%, at least 80%, at least 85%, at least 90%, at least 95%, or 100/6 sequence identity to peptide sequence selected from the group consisting of SEQ ID NOS 5285-5431, and 5996-6084, each of which can be specific to colorectal cancer. The epitope sequence provided herein can have at least 70% sequence identity to peptide sequence selected from the group consisting of SEQ ID NOS 5285-5431, and 5996-6084, each of which can be specific to colorectal cancer.

The tumor expressed proteins provided herein can comprise DLL3, each of which can be specific to glioma. The epitope sequence provided herein can have at least 50%, at least 60%, at least 70%, at least 80%, at least 85%, at least 90%, at least 95%, or 100% sequence identity to peptide sequence selected from the group consisting of SEQ ID NOS 2619-2736, each of which can be specific to glioma. The epitope sequence provided herein can have at least 70% sequence identity to peptide sequence selected from the group consisting of SEQ ID NOS 2619-2736, each of which can be specific to glioma.

The tumor expressed proteins provided herein can comprise MMP13, each of which can be specific to head and neck cancer. The epitope sequence provided herein can have at least 50%, at least 60%, at least 70%, at least 80%, at least 85%, at least 90%, at least 95%, or 100% sequence identity to peptide sequence selected from the group consisting of SEQ ID NOS 4916-5010, each of which can be specific to head and neck cancer. The epitope sequence provided herein can have at least 70% sequence identity to peptide sequence selected from the group consisting of SEQ ID NOS 4916-5010, each of which can be specific to head and neck cancer.

The tumor expressed proteins provided herein can comprise DCAF4L2. SSX1, or a combination thereof, each of which can be specific to liver cancer. The epitope sequence provided herein can have at least 50%, at least 60%, at least 70%, at least 80%, at least 85%, at least 90%, at least 95%, or 100% sequence identity to peptide sequence selected from the group consisting of SEQ ID NOS 2524-2618, and 7359-7448, each of which can be specific to liver cancer. The epitope sequence provided herein can have at least 70% sequence identity to peptide sequence selected from the group consisting of SEQ ID NOS 2524-2618, and 7359-7448, each of which can be specific to liver cancer.

The tumor expressed proteins provided herein can comprise SSX1. MAGEA4, PRAME, CSAG1, MAGEA12, MAGEA2, MAGEC2, PAGE5, PRDM7, SLC45A2, TSPAN10, or any combination thereof, each of which can be specific to melanoma. The epitope sequence provided herein can have at least 50%, at least 60%, at least 70%, at least 80%, at least 85%, at least 90%, at least 95%, or 100% sequence identity to peptide sequence selected from the group consisting of SEQ ID NOS 1868-1963, 4458-4550, 4551-4637,4638-4728, 4729-4816, 5011-5100, 6085-6183, 6184-6307, 7151-7264, 7359-7448, and 8745-8835, each of which can be specific to melanoma. The epitope sequence provided herein can have at least 70% sequence identity to peptide sequence selected from the group consisting of SEQ ID NOS 1868-1963, 4458-4550, 45514637, 46384728, 4729-4816, 5011-5100, 6085-6183, 6184-6307, 7151-7264, 7359-7448, and 8745-8835, each of which can be specific to melanoma.

The tumor expressed proteins provided herein can comprise MAGEA11, MAGEA4, PRAME, or any combination thereof, each of which can be specific to lung squamous cell carcinoma. The epitope sequence provided herein can have at least 50%, at least 60%, at least 70%, at least 80%, at least 85%, at least 90%, at least 95%, or 100% sequence identity to peptide sequence selected from the group consisting of SEQ ID NOS 4368-4457, 4638-4728, and 6085-6183, each of which can be specific to lung squamous cell carcinoma. The epitope sequence provided herein can have at least 70% sequence identity to peptide sequence selected from the group consisting of SEQ ID NOS 4368-4457, 4638-4728, and 6085-6183, each of which can be specific to lung squamous cell carcinoma.

The tumor expressed proteins provided herein can comprise ACTL7A, ACTL7B, ACTL9, ACTRT2, ADAD1, AKAP4, C2orf53, CCDC70, CETN1, DMRTB1, HMGB4, KIF2B, LELP1, PGK2, PRM1, PRM2, SPATA8, TNP1, TPD52L3, UBQLN3, or any combination thereof, each of which can be specific to testis cancer. The epitope sequence provided herein can have at least 50%, at least 60%, at least 70%, at least 80%, at least 85%, at least 90%, at least 95%, or 100% sequence identity to peptide sequence selected from the group consisting of SEQ ID NOS 1-626, 1183-1372, 1566-1658, 2737-2826, 3010-3100, 3197-3319, 4275-4367, 5101-5192, 6308-6486, 7265-7358, 8435-8618, and 8836-8962, each of which can be specific to testis cancer. The epitope sequence provided herein can have at least 70% sequence identity to peptide sequence selected from the group consisting of SEQ ID NOS 1-626, 1183-1372, 1566-1658, 2737-2826, 3010-3100, 3197-3319, 4275-4367, 5101-5192, 6308-6486, 7265-7358, 8435-8618, and 8836-8962, each of which can be specific to testis cancer.

Table 1A provides a summary of numerous peptide sequences that can be tissue-specific antigens, also listed are the HLA alleles that are predicted to bind to the peptide sequences, respectively, as well as the types of cancers that the peptide sequences are specific to, respectively.

Table 1B provides a summary of exemplary, peptide sequences that can be tissue-specific antigens, also listed are the HLA alleles that are predicted to bind to the peptide sequences, respectively. as well as the types of cancers that the peptide sequences are specific to, respectively.

Table 1C provides a summary of exemplary peptide sequences from Table 1B that were validated from mass spectrometry as being presented by antigen presenting cells.

TABLE 1A
Tumor Epitope Sequences

| peptide | allele | gene | cancer | SEQ |
|---|---|---|---|---|
| FNTPAMHIAY | HLA-A01:01 | ACTL7A | Testis | 1 |
| WDTVQDIWEY | HLA-A01:01 | ACTL7A | Testis | 2 |
| NTPAMHIAY | HLA-A01:01 | ACTL7A | Testis | 3 |
| TVQDIWEYLF | HLA-A01:01 | ACTL7A | Testis | 4 |
| LSEHTIRY | HLA-A01:01 | ACTL7A | Testis | 5 |
| VVEVGHGVSY | HLA-A01:01 | ACTL7A | Testis | 6 |
| YEEHGPFFLY | HLA-A01:01 | ACTL7A | Testis | 7 |
| YLLGLINSA | HLA-A02:01 | ACTL7A | Testis | 8 |
| MLFEAFNTPA | HLA-A02:01 | ACTL7A | Testis | 9 |
| YLFRQEMKI | HLA-A02:01 | ACTL7A | Testis | 10 |
| MLSGFPNRL | HLA-A02:01 | ACTL7A | Testis | 11 |
| SLQGFQPLWV | HLA-A02:01 | ACTL7A | Testis | 12 |
| FLCSEMFFKPSL | HLA-A02:01 | ACTL7A | Testis | 13 |
| AYLLGLLNSA | HLA-A02:01 | ACTL7A | Testis | 14 |
| SDLTAYLLGL | HLA-A02:01 | ACTL7A | Testis | 15 |
| ILLCGGSTML | HLA-A02:01 | ACTL7A | Testis | 16 |
| KIAPEEHAV | HLA-A02:01 | ACTL7A | Testis | 17 |
| VLPDGKEIQL | HLA-A02:01 | ACTL7A | Testis | 18 |
| ALDPIEEKKV | HLA-A02:01 | ACTL7A | Testis | 19 |
| YVLPDGKEIQL | HLA-A02:01 | ACTL7A | Testis | 20 |
| VLPERDSAV | HLA-A02:01 | ACTL7A | Testis | 21 |
| RTSGLVVEV | HLA-A02:01 | ACTL7A | Testis | 22 |
| MLSGFPNRLQK | HLA-A03:01 | ACTL7A | Testis | 23 |
| MFFKPSLIK | HLA-A03:01 | ACTL7A | Testis | 24 |
| RFLCSEMFFK | HLA-A03:01 | ACTL7A | Testis | 25 |
| AIMGDGPTKK | HLA-A03:01 | ACTL7A | Testis | 26 |
| AIMGDGPTK | HLA-A03:01 | ACTL7A | Testis | 27 |
| SGFPNRLQK | HLA-A03:01 | ACTL7A | Testis | 28 |
| STMLSGFPNR | HLA-A11:01 | ACTL7A | Testis | 29 |
| FLCSEMFFK | HLA-A11:01 | ACTL7A | Testis | 30 |
| HTIRYVLPDGK | HLA-A11:01 | ACTL7A | Testis | 31 |
| AIMGDGPTKK | HLA-A11:01 | ACTL7A | Testis | 32 |
| AIMGDGPTK | HLA-A11:01 | ACTL7A | Testis | 33 |
| KYAEMLFEAF | HLA-A24:02 | ACTL7A | Testis | 34 |
| RFLCSEMFF | HLA-A24:02 | ACTL7A | Testis | 35 |
| GFQPLWVHRF | HLA-A24:02 | ACTL7A | Testis | 36 |
| EYEEHGPFF | HLA-A24:02 | ACTL7A | Testis | 37 |
| SGFPNRLQK | HLA-A30:01 | ACTL7A | Testis | 38 |
| MFFKPSLIK | HLA-A30:01 | ACTL7A | Testis | 39 |
| QSRLSMYSY | HLA-A30:01 | ACTL7A | Testis | 40 |
| RTSGLVVEV | HLA-A30:01 | ACTL7A | Testis | 41 |
| HTQTVSCLNK | HLA-A30:01 | ACTL7A | Testis | 42 |
| HLKLVNPLR | HLA-A33:03 | ACTL7A | Testis | 43 |
| STMLSGFPNR | HLA-A33:03 | ACTL7A | Testis | 44 |
| TMLSGFPNR | HLA-A33:03 | ACTL7A | Testis | 45 |
| ETAKTGDNR | HLA-A33:03 | ACTL7A | Testis | 46 |
| EHGPFFLYR | HLA-A33:03 | ACTL7A | Testis | 47 |
| VPIYEGYPL | HLA-B07:02 | ACTL7A | Testis | 48 |
| RPKQEVTKAV | HLA-B07:02 | ACTL7A | Testis | 49 |
| FPNRLQKEL | HLA-B07:02 | ACTL7A | Testis | 50 |
| APLQTQAL | HLA-B07:02 | ACTL7A | Testis | 51 |
| LPDGKEIQL | HLA-B07:02 | ACTL7A | Testis | 52 |
| NVHLKLVNPL | HLA-B08:01 | ACTL7A | Testis | 53 |
| HLKLVNPL | HLA-B08:01 | ACTL7A | Testis | 54 |
| TNREKYAEM | HLA-B08:01 | ACTL7A | Testis | 55 |
| DIKKKCCF | HLA-B08:01 | ACTL7A | Testis | 56 |
| DIKKKCCFV | HLA-B08:01 | ACTL7A | Testis | 57 |
| DIALKRDL | HLA-B08:01 | ACTL7A | Testis | 58 |
| SEMFFKPSLI | HLA-B13:02 | ACTL7A | Testis | 59 |
| YEGYPLPSI | HLA-B13:02 | ACTL7A | Testis | 60 |
| SEMFFKPSL | HLA-B13:02 | ACTL7A | Testis | 61 |
| FEYEEHGPFFL | HLA-B13:02 | ACTL7A | Testis | 62 |
| GLPRPTHKI | HLA-B13:02 | ACTL7A | Testis | 63 |
| YLFRQEMKI | HLA-B13:02 | ACTL7A | Testis | 64 |
| RTSGLVVEV | HLA-B13:02 | ACTL7A | Testis | 65 |
| YAGSDLTAY | HLA-B46:01 | ACTL7A | Testis | 66 |
| IAYQSRLSM | HLA-B46:01 | ACTL7A | Testis | 67 |
| YSYGRTSQL | HLA-B46:01 | ACTL7A | Testis | 68 |
| YVVPIYEGY | HLA-B46:01 | ACTL7A | Testis | 69 |
| VEVGHGVSY | HLA-B46:01 | ACTL7A | Testis | 70 |
| YSYGRTSGL | HLA-C01:02 | ACTL7A | Testis | 71 |
| IAPEEHAVL | HLA-C01:02 | ACTL7A | Testis | 72 |
| IAYQSRLSM | HLA-C01:02 | ACTL7A | Testis | 73 |
| VLPERDSAV | HLA-C01:02 | ACTL7A | Testis | 74 |
| QAPLQTQAL | HLA-C01:02 | ACTL7A | Testis | 75 |
| YSYGRTSGL | HLA-C03:04 | ACTL7A | Testis | 76 |
| IAYQSRLSM | HLA-C03:04 | ACTL7A | Testis | 77 |
| SAVWTGGSI | HLA-C03:04 | ACTL7A | Testis | 78 |
| ASLQGFQPL | HLA-C03:04 | ACTL7A | Testis | 79 |
| LAPEEHAVE | HLA-C03:04 | ACTL7A | Testis | 80 |
| FEYEEHGPFF | HLA-C04:01 | ACTL7A | Testis | 81 |
| MWAPPAAIM | HLA-C04:01 | ACTL7A | Testis | 82 |
| YEEHGPFFL | HLA-C04:01 | ACTL7A | Testis | 83 |
| VQDIWEYLF | HLA-C04:01 | ACTL7A | Testis | 84 |
| EYEEHGPFF | HLA-C04:01 | ACTL7A | Testis | 85 |
| VQDIWEYL | HLA-C04:01 | ACTL7A | Testis | 86 |
| YSYGRTSGL | HLA-C07:01 | ACTL7A | Testis | 87 |
| MHIAYQSRL | HLA-C07:01 | ACTL7A | Testis | 88 |
| YSYGRTSGLV | HLA-C07:01 | ACTL7A | Testis | 89 |
| LRHGIIVD | HLA-C07:01 | ACTL7A | Testis | 90 |
| TVQDIWEY | HLA-C07:01 | ACTL7A | Testis | 91 |
| HRFEYEEHG | HLA-C07:01 | ACTL7A | Testis | 92 |
| YSYGRTSGL | HLA-C07:02 | ACTL7A | Testis | 93 |
| MHIAYQSRL | HLA-C07:02 | ACTL7A | Testis | 94 |
| FKPSLIKSM | HLA-C07:02 | ACTL7A | Testis | 95 |
| IAPEEHAVL | HLA-C07:02 | ACTL7A | Testis | 96 |
| SRLSMYSY | HLA-C07:02 | ACTL7A | Testis | 97 |
| VTSQSLLSIY | HLA-A01:01 | ACTL7B | Testis | 98 |
| FTDDHLHII | HLA-A01:01 | ACTL7B | Testis | 99 |
| TSQSLLSIY | HLA-A01:01 | ACTL7B | Testis | 100 |
| IIDLGSQY | HLA-A01:01 | ACTL7B | Testis | 101 |
| FTDDHLHI | HLA-A01:01 | ACTL7B | Testis | 102 |
| YLMQLLNEA | HLA-A02:01 | ACTL7B | Testis | 103 |
| LMFETFGIPA | HLA-A02:01 | ACTL7B | Testis | 104 |
| YAELMFETFGI | HLA-A02:01 | ACTL7B | Testis | 105 |
| FLPEEELGL | HLA-A02:01 | ACTL7B | Testis | 106 |
| AELMFETFGI | HLA-A02:01 | ACTL7B | Testis | 107 |
| ELMFETFGI | HLA-A02:01 | ACTL7B | Testis | 108 |
| FLPEEELGLV | HLA-A02:01 | ACTL7B | Testis | 109 |
| LMFETFGI | HLA-A02:01 | ACTL7B | Testis | 110 |
| NYLMQLLNEA | HLA-A02:01 | ACTL7B | Testis | 111 |
| KILPEEHAV | HLA-A02:01 | ACTL7B | Testis | 112 |
| GLVPEELRV | HLA-A02:01 | ACTL7B | Testis | 113 |
| ILPEEHAVLV | HLA-A02:01 | ACTL7B | Testis | 114 |
| ILPEEHAVL | HLA-A02:01 | ACTL7B | Testis | 115 |
| SLAGSTQPGL | HLA-A02:01 | ACTL7B | Testis | 116 |
| LLCPGDSPAV | HLA-A02:01 | ACTL7B | Testis | 117 |
| SLLSIYSYGK | HLA-A03:01 | ACTL7B | Testis | 118 |
| KMKPRKVHK | HLA-A03:01 | ACTL7B | Testis | 119 |
| LLSIYSYGK | HLA-A03:01 | ACTL7B | Testis | 120 |
| AVAAAPERK | HLA-A03:01 | ACTL7B | Testis | 121 |
| HIIEHIKKK | HLA-A03:01 | ACTL7B | Testis | 122 |
| SLLSIYSYGK | HLA-A11:01 | ACTL7B | Testis | 123 |
| AVAAAPERK | HLA-A11:01 | ACTL7B | Testis | 124 |
| LLSIYSYGK | HLA-A11:01 | ACTL7B | Testis | 125 |
| VIIDLGSQYCK | HLA-A11:01 | ACTL7B | Testis | 126 |
| AFQQLWVSK | HLA-A11:01 | ACTL7B | Testis | 127 |
| KYAELMFETF | HLA-A24:02 | ACTL7B | Testis | 128 |
| RFRCSEMLF | HLA-A24:02 | ACTL7B | Testis | 129 |
| EKYAELMFETF | HLA-A24:02 | ACTL7B | Testis | 130 |
| GYAGEPRPTYF | HLA-A24:02 | ACTL7B | Testis | 131 |
| AFTDDHLHI | HLA-A24:02 | ACTL7B | Testis | 132 |
| KMKPRKVHK | HLA-A30:01 | ACTL7B | Testis | 133 |
| ATRNSPMPL | HLA-A30:01 | ACTL7B | Testis | 134 |
| MATRNSPMPL | HLA-A30:01 | ACTL7B | Testis | 135 |
| KIKAVIIDL | HLA-A30:01 | ACTL7B | Testis | 136 |
| HIIEHIKKK | HLA-A30:01 | ACTL7B | Testis | 137 |
| TMLDGFPER | HLA-A33:03 | ACTL7B | Testis | 138 |
| DVLPGLTSR | HLA-A33:03 | ACTL7B | Testis | 139 |
| CTMLDGFPER | HLA-A33:03 | ACTL7B | Testis | 140 |
| EAADAGDTR | HLA-A33:03 | ACTL7B | Testis | 141 |
| APLKLVNPL | HLA-B07:02 | ACTL7B | Testis | 142 |
| RPTYFISSTV | HLA-B07:02 | ACTL7B | Testis | 143 |
| FPERFQREL | HLA-B07:02 | ACTL7B | Testis | 144 |
| SPMPLGTAQ | HLA-B07:02 | ACTL7B | Testis | 145 |
| VPISEGDVL | HLA-B07:02 | ACTL7B | Testis | 146 |
| SNREKYAEL | HLA-B08:01 | ACTL7B | Testis | 147 |
| HIKKKCCYA | HLA-B08:01 | ACTL7B | Testis | 148 |
| HIKKKCCYAAF | HLA-B08:01 | ACTL7B | Testis | 149 |
| AGDTRKWTL | HLA-B08:01 | ACTL7B | Testis | 150 |
| AAPERKTSV | HLA-B08:01 | ACTL7B | Testis | 151 |
| ERFQRELSL | HLA-B08:01 | ACTL7B | Testis | 152 |
| AELMFETFGI | HLA-B13:02 | ACTL7B | Testis | 153 |
| SEMLFQPSL | HLA-B13:02 | ACTL7B | Testis | 154 |
| WEYIFRTAMKI | HLA-B13:02 | ACTL7B | Testis | 155 |
| GLVPEELRV | HLA-B13:02 | ACTL7B | Testis | 156 |
| YIFRTAMKI | HLA-B13:02 | ACTL7B | Testis | 157 |
| HLHIIEHI | HLA-B13:02 | ACTL7B | Testis | 158 |
| YSYGKTSGL | HLA-B46:01 | ACTL7B | Testis | 159 |
| LLNEAGHAF | HLA-B46:01 | ACTL7B | Testis | 160 |
| SILASLQAF | HLA-B46:01 | ACTL7B | Testis | 161 |
| VIIDLGSQY | HLA-B46:01 | ACTL7B | Testis | 162 |
| YAGEPRPTY | HLA-B46:01 | ACTL7B | Testis | 163 |
| AMHVTSQSL | HLA-B46:01 | ACTL7B | Testis | 164 |
| YSYGKTSGL | HLA-C01:02 | ACTL7B | Testis | 165 |
| ILPEEHAVL | HLA-C01:02 | ACTL7B | Testis | 166 |
| FTDDHLHII | HLA-C01:02 | ACTL7B | Testis | 167 |
| AAPERKTSV | HLA-C01:02 | ACTL7B | Testis | 168 |
| FLPEEELGL | HLA-C01:02 | ACTL7B | Testis | 169 |
| YSYGKTSGL | HLA-C03:04 | ACTL7B | Testis | 170 |
| MATRNSPMPL | HLA-C03:04 | ACTL7B | Testis | 171 |
| SVWTGGSIL | HLA-C03:04 | ACTL7B | Testis | 172 |
| STQPGLPEL | HLA-C03:04 | ACTL7B | Testis | 173 |
| AAFLPEEEL | HLA-C03:04 | ACTL7B | Testis | 174 |
| FTDDHLHIL | HLA-C04:01 | ACTL7B | Testis | 175 |
| MLDGFPERF | HLA-C04:01 | ACTL7B | Testis | 176 |
| RFRCSEMLF | HLA-C04:01 | ACTL7B | Testis | 177 |
| VQDIWEYIF | HLA-C04:01 | ACTL7B | Testis | 178 |
| ERFQRELSI | HLA-C07:01 | ACTL7B | Testis | 179 |
| YSYGKTSGL | HLA-C07:01 | ACTL7B | Testis | 180 |
| ERFRCSEML | HLA-C07:01 | ACTL7B | Testis | 181 |
| TRPGPDASL | HLA-C07:01 | ACTL7B | Testis | 182 |
| LRVDYELPD | HLA-C07:01 | ACTL7B | Testis | 183 |
| YSYGKTSGL | HLA-C07:02 | ACTL7B | Testis | 184 |
| ERFQRELSI | HLA-C07:02 | ACTL7B | Testis | 185 |
| RFRCSEMLF | HLA-C07:02 | ACTL7B | Testis | 186 |
| TRPGPDASL | HLA-C07:02 | ACTL7B | Testis | 187 |
| ILPEEHAVL | HLA-C07:02 | ACTL7B | Testis | 188 |
| TRNSPMPL | HLA-C07:02 | ACTL7B | Testis | 189 |
| VVDTGHGVTY | HLA-A01:01 | ACTL9 | Testis | 190 |
| YTVPVFQGY | HLA-A01:01 | ACTL9 | Testis | 191 |
| ASQSVLSVY | HLA-A01:01 | ACTL9 | Testis | 192 |
| LVENIKHHY | HLA-A01:01 | ACTL9 | Testis | 193 |
| FLAEMLLQA | HLA-A02:01 | ACTL9 | Testis | 194 |
| HLLEHDLRV | HLA-A02:01 | ACTL9 | Testis | 195 |
| FLAEMLLQAGL | HLA-A02:01 | ACTL9 | Testis | 196 |
| KLVEVAFESL | HLA-A02:01 | ACTL9 | Testis | 197 |
| MLLQAGLPL | HLA-A02:01 | ACTL9 | Testis | 198 |
| AMYVASQSV | HLA-A02:01 | ACTL9 | Testis | 199 |
| AFLAEMLLQA | HLA-A02:01 | ACTL9 | Testis | 200 |
| RVLPELTLV | HLA-A02:01 | ACTL9 | Testis | 201 |
| AFLAEMLLQAGL | HLA-A02:01 | ACTL9 | Testis | 202 |
| KLPDGRTVTL | HLA-A02:01 | ACTL9 | Testis | 203 |
| ELLFNPPEV | HLA-A02:01 | ACTL9 | Testis | 204 |
| ALPAETHVV | HLA-A02:01 | ACTL9 | Testis | 205 |
| LLFNPPEV | HLA-A02:01 | ACTL9 | Testis | 206 |
| STMAKQSLRK | HLA-A03:01 | ACTL9 | Testis | 207 |
| TMAKQSLRK | HLA-A03:01 | ACTL9 | Testis | 208 |
| GMVADRLPPK | HLA-A03:01 | ACTL9 | Testis | 209 |
| MVADRLPPK | HLA-A03:01 | ACTL9 | Testis | 210 |
| VVIDMGTGTCK | HLA-A03:01 | ACTL9 | Testis | 211 |
| STMAKQSLRK | HLA-A11:01 | ACTL9 | Testis | 212 |
| MVADRLPPK | HLA-A11:01 | ACTL9 | Testis | 213 |
| ATILGCQPK | HLA-A11:01 | ACTL9 | Testis | 214 |
| VVIDMGTGTCK | HLA-A11:01 | ACTL9 | Testis | 215 |
| ATILGCQPKK | HLA-A11:01 | ACTL9 | Testis | 216 |
| LFQCPELLF | HLA-A24:02 | ACTL9 | Testis | 217 |
| HYCYVASDF | HLA-A24:02 | ACTL9 | Testis | 218 |
| MYVASQSVL | HLA-A24:02 | ACTL9 | Testis | 219 |
| HHYCYVASDF | HLA-A24:02 | ACTL9 | Testis | 220 |
| QYEEQGPYI | HLA-A24:02 | ACTL9 | Testis | 221 |
| RFRAELLRA | HLA-A30:01 | ACTL9 | Testis | 222 |
| SLRSPAMYV | HLA-A30:01 | ACTL9 | Testis | 223 |
| MVADRLPPK | HLA-A30:01 | ACTL9 | Testis | 224 |
| ASRPKSSES | HLA-A30:01 | ACTL9 | Testis | 225 |
| ATILGCQPK | HLA-A30:01 | ACTL9 | Testis | 226 |
| NIKHHYCYV | HLA-A30:01 | ACTL9 | Testis | 227 |
| STMAKQSLR | HLA-A33:03 | ACTL9 | Testis | 228 |
| QTFIGEAAR | HLA-A33:03 | ACTL9 | Testis | 229 |
| SVLSVYAHGR | HLA-A33:03 | ACTL9 | Testis | 230 |
| EEQGPYIVYR | HLA-A33:03 | ACTL9 | Testis | 231 |
| EQGPYIVYR | HLA-A33:03 | ACTL9 | Testis | 232 |
| NVVNKPLQR | HLA-A33:03 | ACTL9 | Testis | 233 |
| SPNVVNKPL | HLA-B07:02 | ACTL9 | Testis | 234 |
| KPATSGQSGL | HLA-B07:02 | ACTL9 | Testis | 235 |
| RPEQEYKRTL | HLA-B07:02 | ACTL9 | Testis | 236 |
| LPDGRTVTL | HLA-B07:02 | ACTL9 | Testis | 237 |
| SPATNREKL | HLA-B07:02 | ACTL9 | Testis | 238 |
| MAKQSLRKLSL | HLA-B08:01 | ACTL9 | Testis | 239 |
| NIKHHYCYV | HLA-B08:01 | ACTL9 | Testis | 240 |
| TMAKQSLRKLSL | HLA-B08:01 | ACTL9 | Testis | 241 |
| KQSLRKLSL | HLA-B08:01 | ACTL9 | Testis | 242 |
| ELIWRHLL | HLA-B08:01 | ACTL9 | Testis | 243 |
| EQEYKRTL | HLA-B08:01 | ACTL9 | Testis | 244 |
| VDWDAAELI | HLA-B13:02 | ACTL9 | Testis | 245 |
| VVDWDAAELI | HLA-B13:02 | ACTL9 | Testis | 246 |
| AELIWRHLL | HLA-B13:02 | ACTL9 | Testis | 247 |
| GQSGLQTFI | HLA-B13:02 | ACTL9 | Testis | 248 |
| GQASPTYTV | HLA-B13:02 | ACTL9 | Testis | 249 |
| RVLPELTLV | HLA-B13:02 | ACTL9 | Testis | 250 |
| YTVPVFQGY | HLA-B46:01 | ACTL9 | Testis | 251 |
| FAGQASPTY | HLA-B46:01 | ACTL9 | Testis | 252 |
| FSVWIGGSI | HLA-B46:01 | ACTL9 | Testis | 253 |
| VAAQPTRNF | HLA-B46:01 | ACTL9 | Testis | 254 |
| VLREQYEEQ | HLA-B46:01 | ACTL9 | Testis | 255 |
| ASRPKSSES | HLA-B46:01 | ACTL9 | Testis | 256 |
| MLLQAGLPL | HLA-C01:02 | ACTL9 | Testis | 257 |
| LSPVGLSTM | HLA-C01:02 | ACTL9 | Testis | 258 |
| FIGEAARVL | HLA-C01:02 | ACTL9 | Testis | 259 |
| KLPDGRTVTL | HLA-C01:02 | ACTL9 | Testis | 260 |
| FSPATNREK | HLA-C01:02 | ACTL9 | Testis | 261 |
| YAHGRVSGL | HLA-C03:04 | ACTL9 | Testis | 262 |
| MLLQAGLPL | HLA-C03:04 | ACTL9 | Testis | 263 |
| FSVWIGGSI | HLA-C03:04 | ACTL9 | Testis | 264 |
| FIGEAARVL | HLA-C03:04 | ACTL9 | Testis | 265 |
| FSDPPFSPA | HLA-C03:04 | ACTL9 | Testis | 266 |
| LFQCPELLF | HLA-C04:01 | ACTL9 | Testis | 267 |
| FSDPPFSPA | HLA-C04:01 | ACTL9 | Testis | 268 |
| RLDLAGNHL | HLA-C04:01 | ACTL9 | Testis | 269 |
| VVDWDAAEL | HLA-C04:01 | ACTL9 | Testis | 270 |
| HYCYVASDF | HLA-C04:01 | ACTL9 | Testis | 271 |
| FRAELLRAL | HLA-C07:01 | ACTL9 | Testis | 272 |
| MRADLAQNV | HLA-C07:01 | ACTL9 | Testis | 273 |
| MRADLAQNVL | HLA-C07:01 | ACTL9 | Testis | 274 |
| LRSGIVVD | HLA-C07:01 | ACTL9 | Testis | 275 |
| RAFQSCWVL | HLA-C07:01 | ACTL9 | Testis | 276 |
| RSGIVVDWD | HLA-C07:01 | ACTL9 | Testis | 277 |
| FRAELLRAL | HLA-C07:02 | ACTL9 | Testis | 278 |
| ATHDHPLLF | HLA-C07:02 | ACTL9 | Testis | 279 |
| MYVASQSVL | HLA-C07:02 | ACTL9 | Testis | 280 |
| ARVLPELTL | HLA-C07:02 | ACTL9 | Testis | 281 |
| FNPPEVPGL | HLA-C07:02 | ACTL9 | Testis | 282 |
| LSDQAVLALY | HLA-A01:01 | ACTRT2 | Testis | 283 |
| YLSDQAVLALY | HLA-A01:01 | ACTRT2 | Testis | 284 |
| FYLSDQAVLALY | HLA-A01:01 | ACTRT2 | Testis | 285 |
| DTDIQKILF | HLA-A01:01 | ACTRT2 | Testis | 286 |
| LSDQAVLAL | HLA-A01:01 | ACTRT2 | Testis | 287 |
| HLFEWELGV | HLA-A02:01 | ACTRT2 | Testis | 288 |
| KHLFEWELGV | HLA-A02:01 | ACTRT2 | Testis | 289 |
| KMAEVMFENFGV | HLA-A02:01 | ACTRT2 | Testis | 290 |
| YLSDQAVLAL | HLA-A02:01 | ACTRT2 | Testis | 291 |
| YLSDQAVLA | HLA-A02:01 | ACTRT2 | Testis | 292 |
| WKHLFEWELGV | HLA-A02:01 | ACTRT2 | Testis | 293 |
| SLSSFKQMWV | HLA-A02:01 | ACTRT2 | Testis | 294 |
| VMFENFGVPA | HLA-A02:01 | ACTRT2 | Testis | 295 |
| LWKHLFEWELGV | HLA-A02:01 | ACTRT2 | Testis | 296 |
| KLPDQNIISL | HLA-A02:01 | ACTRT2 | Testis | 297 |
| SLGDPLHQA | HLA-A02:01 | ACTRT2 | Testis | 298 |
| RLLKELEQL | HLA-A02:01 | ACTRT2 | Testis | 299 |
| GLDDRLLKEL | HLA-A02:01 | ACTRT2 | Testis | 300 |
| SLPHAVTKL | HLA-A02:01 | ACTRT2 | Testis | 301 |
| GLDDRLLKE | HLA-A02:01 | ACTRT2 | Testis | 302 |
| IVTSLSSFK | HLA-A03:01 | ACTRT2 | Testis | 303 |
| SIVTSLSSFK | HLA-A03:01 | ACTRT2 | Testis | 304 |
| TLFHGLDDRLLK | HLA-A03:01 | ACTRI2 | Testis | 305 |
| VIFDNGSGFCK | HLA-A03:01 | ACTRT2 | Testis | 306 |
| HTFPCQLDK | HLA-A03:01 | ACTRT2 | Testis | 307 |
| HLFEWELGVK | HLA-A03:01 | ACTRT2 | Testis | 308 |
| SIVTSLSSFK | HLA-A11:01 | ACTRT2 | Testis | 309 |
| IVTSLSSFK | HLA-A11:01 | ACTRT2 | Testis | 310 |
| HTFPCQLDK | HLA-A11:01 | ACTRT2 | Testis | 311 |
| VIFDNGSGFCK | HLA-A11:01 | ACTRT2 | Testis | 312 |
| YSLPHAVTK | HLA-A11:01 | ACTRT2 | Testis | 313 |
| KMAEVMFENF | HLA-A24:02 | ACTRT2 | Testis | 314 |
| RLWKHLFEW | HLA-A24:02 | ACTRT2 | Testis | 315 |
| LYASACVTQL | HLA-A24:02 | ACTRT2 | Testis | 316 |
| FYLSDQAVL | HLA-A24:02 | ACTRT2 | Testis | 317 |
| VLSGGTTLF | HLA-A24:02 | ACTRT2 | Testis | 318 |
| RWFSTWIGA | HLA-A30:01 | ACTRT2 | Testis | 319 |
| YSLPHAVTK | HLA-A30:01 | ACTRT2 | Testis | 320 |
| HTFPCQLDK | HLA-A30:01 | ACTRT2 | Testis | 321 |
| ITKCDTDIQK | HLA-A30:01 | ACTRT2 | Testis | 322 |
| GLVDDIKKK | HLA-A30:01 | ACTRT2 | Testis | 323 |
| VTKLHVAGR | HLA-A33:03 | ACTRT2 | Testis | 324 |
| EFGTSVVQR | HLA-A33:03 | ACTRT2 | Testis | 325 |
| EFGTSVVQRR | HLA-A33:03 | ACTRT2 | Testis | 326 |
| AVTKLHVAGR | HLA-A33:03 | ACTRT2 | Testis | 327 |
| DFKEFGTSVVQR | HLA-A33:03 | ACTRT2 | Testis | 328 |
| GPRHMVSSI | HLA-B07:02 | ACTRT2 | Testis | 329 |
| FGPRHMVSSI | HLA-B07:02 | ACTRT2 | Testis | 330 |
| VPIFEGYSL | HLA-B07:02 | ACTRT2 | Testis | 331 |
| LPDGNIISL | HLA-B07:02 | ACTRT2 | Testis | 332 |
| QPLLATEPSL | HLA-B07:02 | ACTRT2 | Testis | 333 |
| DIKKKLCYV | HLA-B08:01 | ACTRT2 | Testis | 334 |
| FGPRHMVSSI | HLA-B08:01 | ACTRT2 | Testis | 335 |
| DIKKKLCYVAL | HLA-B08:01 | ACTRT2 | Testis | 336 |
| EANQKKYFV | HLA-B08:01 | ACTRT2 | Testis | 337 |
| EALYKQEAL | HLA-B08:01 | ACTRT2 | Testis | 338 |
| ADFKEFGTSV | HLA-B13:02 | ACTRT2 | Testis | 339 |
| AEVMFENFGV | HLA-B13:02 | ACTRT2 | Testis | 340 |
| FEGYSLPHAV | HLA-B13:02 | ACTRT2 | Testis | 341 |
| HQAPEALFV | HLA-B13:02 | ACTRT2 | Testis | 342 |
| HLFEWELGV | HLA-B13:02 | ACTRT2 | Testis | 343 |
| KCDTDIQKI | HLA-B13:02 | ACTRT2 | Testis | 344 |
| SIVTSLSSF | HLA-B46:01 | ACTRT2 | Testis | 345 |
| FSTWIGASI | HLA-B46:01 | ACTRT2 | Testis | 346 |
| YASACVTGL | HLA-B46:01 | ACTRT2 | Testis | 347 |
| VIFDNGSGF | HLA-B46:01 | ACTRT2 | Testis | 348 |
| SSIVGHLKF | HLA-B46:01 | ACTRT2 | Testis | 349 |
| ASIVTSLSSF | HLA-B46:01 | ACTRT2 | Testis | 350 |
| LSDQAVLAL | HLA-C01:02 | ACTRI2 | Testis | 351 |
| YASACVTGL | HLA-C01:02 | ACTRT2 | Testis | 352 |
| FSTWIGASI | HLA-C01:02 | ACTRT2 | Testis | 353 |
| KLPDGNIISL | HLA-C01:02 | ACTRT2 | Testis | 354 |
| SLPHAVTKL | HLA-C01:02 | ACTRT2 | Testis | 355 |
| FGPRHMVSSI | HLA-C01:02 | ACTRT2 | Testis | 356 |
| YASACVTGL | HLA-C03:04 | ACTRT2 | Testis | 357 |
| FSTWIGASI | HLA-C03:04 | ACTRT2 | Testis | 358 |
| IVLSGGTTL | HLA-C03:04 | ACTRT2 | Testis | 359 |
| HALDSPAVI | HLA-C03:04 | ACTRT2 | Testis | 360 |
| VALEPEKEL | HLA-C03:04 | ACTRT2 | Testis | 361 |
| LSDQAVLAL | HLA-C03:04 | ACTRT2 | Testis | 362 |
| FYLSDQAVLAL | HLA-C04:01 | ACTRT2 | Testis | 363 |
| LHQAPEALF | HLA-C04:01 | ACTRT2 | Testis | 364 |
| ALDSPAVIF | HLA-C04:01 | ACTRT2 | Testis | 365 |
| IFDNGSGF | HLA-C04:01 | ACTRT2 | Testis | 366 |
| IFDNGSGFC | HLA-C04:01 | ACTRT2 | Testis | 367 |
| FSTWIGASI | HLA-C07:01 | ACTRT2 | Testis | 368 |
| NREKMAEVM | HLA-C07:01 | ACTRT2 | Testis | 369 |
| FYLSDQAVL | HLA-C07:01 | ACTRT2 | Testis | 370 |
| RRPEEVLRE | HLA-C07:01 | ACTRT2 | Testis | 371 |
| RRPEEVLREY | HLA-C07:01 | ACTRT2 | Testis | 372 |
| SRRPEEVL | HLA-C07:01 | ACTRT2 | Testis | 373 |
| FYLSDQAVL | HLA-C07:02 | ACTRT2 | Testis | 374 |
| LYKQEALQL | HLA-C07:02 | ACTRT2 | Testis | 375 |
| KYFVGEEAL | HLA-C07:02 | ACTRT2 | Testis | 376 |
| RRPEEVLREY | HLA-C07:02 | ACTRT2 | Testis | 377 |
| SRRPEEVL | HLA-C07:02 | ACTRT2 | Testis | 378 |
| LTSKLPMFY | HLA-A01:01 | ADAD1 | Testis | 379 |
| ALTSKLPMFY | HLA-A01:01 | ADAD1 | Testis | 380 |
| VTARRSLLRY | HLA-A01:01 | ADAD1 | Testis | 381 |
| LSELAYVSKVHY | HLA-A01:01 | ADAD1 | Testis | 382 |
| LLSHFIQPV | HLA-A02:01 | ADAD1 | Testis | 383 |
| ALLSHFIQPV | HLA-A02:01 | ADAD1 | Testis | 384 |
| VVLSELAYV | HLA-A02:01 | ADAD1 | Testis | 385 |
| KMASKVTQV | HLA-A02:01 | ADAD1 | Testis | 386 |
| SLVPSAYPL | HLA-A02:01 | ADAD1 | Testis | 387 |
| KLALDELLQL | HLA-A02:01 | ADAD1 | Testis | 388 |
| NLSSISNPV | HLA-A02:01 | ADAD1 | Testis | 389 |
| SLLRYFYRQL | HLA-A02:01 | ADAD1 | Testis | 390 |
| MLSRFNLLA | HLA-A02:01 | ADAD1 | Testis | 391 |
| AMLSRFNLL | HLA-A02:01 | ADAD1 | Testis | 392 |
| AMMEKSIFCT | HLA-A02:01 | ADAD1 | Testis | 393 |
| ALDELLQL | HLA-A02:01 | ADAD1 | Testis | 394 |
| FIIERAGQHEV | HLA-A02:01 | ADAD1 | Testis | 395 |
| VLHDTHAVV | HLA-A02:01 | ADAD1 | Testis | 396 |
| VLSELAYV | HLA-A02:01 | ADAD1 | Testis | 397 |
| QLDEPEPRI | HLA-A02:01 | ADAD1 | Testis | 398 |
| ALDELLQLD | HLA-A02:01 | ADAD1 | Testis | 399 |
| MLSRFNLLAK | HLA-A03:01 | ADAD1 | Testis | 400 |
| CLYMNQLPK | HLA-A03:01 | ADAD1 | Testis | 401 |
| GMSMASRLCK | HLA-A03:01 | ADAD1 | Testis | 402 |
| RVDDALTSK | HLA-A03:01 | ADAD1 | Testis | 403 |
| AVVDGIQYK | HLA-A03:01 | ADAD1 | Testis | 404 |
| MSMASRLCK | HLA-A11:01 | ADAD1 | Testis | 405 |
| SISNPVLPPK | HLA-A11:01 | ADAD1 | Testis | 406 |
| AVVDGIQYK | HLA-A11:01 | ADAD1 | Testis | 407 |
| SSLAAFIIER | HLA-A11:01 | ADAD1 | Testis | 408 |
| VVLSELAYVSK | HLA-A11:01 | ADAD1 | Testis | 409 |
| RYFYRQLLLF | HLA-A24:02 | ADAD1 | Testis | 410 |
| RYFYRQLLL | HLA-A24:02 | ADAD1 | Testis | 411 |
| YFYRQLLLF | HLA-A24:02 | ADAD1 | Testis | 412 |
| KYSSSLAAF | HLA-A24:02 | ADAD1 | Testis | 413 |
| MSMASRLCK | HLA-A30:01 | ADAD1 | Testis | 414 |
| LSRFNLLAK | HLA-A30:01 | ADAD1 | Testis | 415 |
| TARRSLLSK | HLA-A30:01 | ADAD1 | Testis | 416 |
| AVVDGIQYK | HLA-A30:01 | ADAD1 | Testis | 417 |
| KIPKEFIMK | HLA-A30:01 | ADAD1 | Testis | 418 |
| RQLLLFYSK | HLA-A30:01 | ADAD1 | Testis | 419 |
| DTHAVVTAR | HLA-A33:03 | ADAD1 | Testis | 420 |
| YVSKVHYEGR | HLA-A33:03 | ADAD1 | Testis | 421 |
| RSLLRYFYR | HLA-A33:03 | ADAD1 | Testis | 422 |
| RFNQLISNR | HLA-A33:03 | ADAD1 | Testis | 423 |
| ERFNQLISNR | HLA-A33:03 | ADAD1 | Testis | 424 |
| SPFKSGMSM | HLA-B07:02 | ADAD1 | Testis | 425 |
| SSPFKSGMSM | HLA-B07:02 | ADAD1 | Testis | 426 |
| QPVYISSIL | HLA-B07:02 | ADAD1 | Testis | 427 |
| IGPYFAFCAV | HLA-B07:02 | ADAD1 | Testis | 428 |
| FPAEPVVL | HLA-B07:02 | ADAD1 | Testis | 429 |
| IVKERFNQL | HLA-B08:01 | ADAD1 | Testis | 430 |
| NLEYKFLSL | HLA-B08:01 | ADAD1 | Testis | 431 |
| AMLSRFNLL | HLA-B08:01 | ADAD1 | Testis | 432 |
| DIKPDGRVL | HLA-B08:01 | ADAD1 | Testis | 433 |
| SELAYVSKV | HLA-B13:02 | ADAD1 | Testis | 434 |
| VEGKIYLTV | HLA-B13:02 | ADAD1 | Testis | 435 |
| FAFCAVVDGI | HLA-B13:02 | ADAD1 | Testis | 436 |
| AQGDVSLEI | HLA-B13:02 | ADAD1 | Testis | 437 |
| HQFAQMQRV | HLA-B13:02 | ADAD1 | Testis | 438 |
| QQHGYGSWI | HLA-B13:02 | ADAD1 | Testis | 439 |
| FQSSQVPSF | HLA-B46:01 | ADAD1 | Testis | 440 |
| LTSKLPMFY | HLA-B46:01 | ADAD1 | Testis | 441 |
| LSHFIQPVY | HLA-B46:01 | ADAD1 | Testis | 442 |
| AAKCMSASY | HLA-B46:01 | ADAD1 | Testis | 443 |
| IPKEFIMKY | HLA-B46:01 | ADAD1 | Testis | 444 |
| EAKCKLKSY | HLA-B46:01 | ADAD1 | Testis | 445 |
| SAYPLQMNL | HLA-C01:02 | ADAD1 | Testis | 446 |
| YSSSLAAFI | HLA-C01:02 | ADAD1 | Testis | 447 |
| SLVPSAYPL | HLA-C01:02 | ADAD1 | Testis | 448 |
| QVPSFAQML | HLA-C01:02 | ADAD1 | Testis | 449 |
| SSPFKSGMSM | HLA-C01:02 | ADAD1 | Testis | 450 |
| AEPVVLSEL | HLA-C01:02 | ADAD1 | Testis | 451 |
| SAYPLQMNL | HLA-C03:04 | ADAD1 | Testis | 452 |
| SAFEANEEL | HLA-C03:04 | ADAD1 | Testis | 453 |
| VAVEGKIYL | HLA-C03:04 | ADAD1 | Testis | 454 |
| FAQMQRVQL | HLA-C03:04 | ADAD1 | Testis | 455 |
| FYSKNPAMM | HLA-C04:01 | ADAD1 | Testis | 456 |
| RYFYRQLLL | HLA-C04:01 | ADAD1 | Testis | 457 |
| YSSSLAAFI | HLA-C04:01 | ADAD1 | Testis | 458 |
| PFPAEPVVL | HLA-C04:01 | ADAD1 | Testis | 459 |
| ALDELLQL | HLA-C04:01 | ADAD1 | Testis | 460 |
| LRYFYRQLL | HLA-C07:01 | ADAD1 | Testis | 461 |
| LRYFYRQLLL | HLA-C07:01 | ADAD1 | Testis | 462 |
| RYFYRQLLL | HLA-C07:01 | ADAD1 | Testis | 463 |
| TRWEVLGVQ | HLA-C07:01 | ADAD1 | Testis | 464 |
| LRYFYRQL | HLA-C07:01 | ADAD1 | Testis | 465 |
| RRSLLRYF | HLA-C07:01 | ADAD1 | Testis | 466 |
| LRYFYRQLL | HLA-C07:02 | ADAD1 | Testis | 467 |
| RYFYRQLLL | HLA-C07:02 | ADAD1 | Testis | 468 |
| FYSKNPAMM | HLA-C07:02 | ADAD1 | Testis | 469 |
| IKPDGRVL | HLA-C07:02 | ADAD1 | Testis | 470 |
| HYEGRHIQY | HLA-C07:02 | ADAD1 | Testis | 471 |
| YADQVNIDY | HLA-A01:01 | AKAP4 | Testis | 472 |
| FADSISKGLMVY | HLA-A01:01 | AKAP4 | Testis | 473 |
| YSVYADQVNIDY | HLA-A01:01 | AKAP4 | Testis | 474 |
| SSENCYSVY | HLA-A01:01 | AKAP4 | Testis | 475 |
| CSIDDLSFY | HLA-A01:01 | AKAP4 | Testis | 476 |
| YSDTTMMSD | HLA-A01:01 | AKAP4 | Testis | 477 |
| ASQFNVPMLY | HLA-A01:01 | AKAP4 | Testis | 478 |
| SIDDLSFYV | HLA-A02:01 | AKAP4 | Testis | 479 |
| VLMTDSDFV | HLA-A02:01 | AKAP4 | Testis | 480 |
| MMSDDIDWL | HLA-A02:01 | AKAP4 | Testis | 481 |
| GQLEKLPQV | HLA-A02:01 | AKAP4 | Testis | 482 |
| YLMNRPQNL | HLA-A02:01 | AKAP4 | Testis | 483 |
| LLDWLLANL | HLA-A02:01 | AKAP4 | Testis | 484 |
| FIDKLVESV | HLA-A02:01 | AKAP4 | Testis | 485 |
| GLMVYANQV | HLA-A02:01 | AKAP4 | Testis | 486 |
| KLVESVMKL | HLA-A02:01 | AKAP4 | Testis | 487 |
| QLLDWLLANL | HLA-A02:01 | AKAP4 | Testis | 488 |
| VLMTDSDFVSAV | HLA-A02:01 | AKAP4 | Testis | 420 |
| MDMSNIVLML | HLA-A02:01 | AKAP4 | Testis | 490 |
| WIAASQFNV | HLA-A02:01 | AKAP4 | Testis | 491 |
| ALAELEEQA | HLA-A02:01 | AKAP4 | Testis | 492 |
| SLEEKEIIV | HLA-A02:01 | AKAP4 | Testis | 493 |
| SLQKQLQAV | HLA-A02:01 | AKAP4 | Testis | 494 |
| ALAELEEQAA | HLA-A02:01 | AKAP4 | Testis | 495 |
| YQDSLGHEV | HLA-A02:01 | AKAP4 | Testis | 496 |
| RISPRTPASK | HLA-A03:01 | AKAP4 | Testis | 497 |
| MMVSLMKTLK | HLA-A03:01 | AKAP4 | Testis | 498 |
| FLYSELSNK | HLA-A03:01 | AKAP4 | Testis | 499 |
| SFLYSELSNK | HLA-A03:01 | AKAP4 | Testis | 500 |
| VMKLCLIMAK | HLA-A03:01 | AKAP4 | Testis | 501 |
| KLLNENPFK | HLA-A03:01 | AKAP4 | Testis | 502 |
| ALSPSTSTCK | HLA-A03:01 | AKAP4 | Testis | 503 |
| SQFNVPMLY | HLA-A03:01 | AKAP4 | Testis | 504 |
| ASDMMVSLMK | HLA-A11:01 | AKAP4 | Testis | 505 |
| SQSLSYASLK | HLA-A11:01 | AKAP4 | Testis | 506 |
| ATDIMEAMLK | HLA-A11:01 | AKAP4 | Testis | 507 |
| QSLSYASLK | HLA-A11:01 | AKAP4 | Testis | 508 |
| SSLVIQMAHK | HLA-A11:01 | AKAP4 | Testis | 509 |
| SVMKLCLIMAK | HLA-A11:01 | AKAP4 | Testis | 510 |
| SICPSPGNK | HLA-A11:01 | AKAP4 | Testis | 511 |
| ASANKPNFR | HLA-A11:01 | AKAP4 | Testis | 512 |
| VVLKRVLLR | HLA-A11:01 | AKAP4 | Testis | 513 |
| SVLNWLLSDLQK | HLA-A11:0] | AKAP4 | Testis | 514 |
| FYVNRLSSL | HLA-A24:02 | AKAP4 | Testis | 515 |
| KYALGFQHAL | HLA-A24:02 | AKAP4 | Testis | 516 |
| SQFNVPMLYF | HLA-A24:02 | AKAP4 | Testis | 517 |
| SFYVNRLSSL | HLA-A24:02 | AKAP4 | Testis | 518 |
| FYVNRLSSLV | HLA-A24:02 | AKAP4 | Testis | 519 |
| VYADQVNIDYL | HLA-A24:02 | AKAP4 | Testis | 520 |
| KYSNDGAAL | HLA-A24:02 | AKAP4 | Testis | 521 |
| QFNVPMLYF | HLA-A24:02 | AKAP4 | Testis | 522 |
| NYQDSLGHEVI | HLA-A24:02 | AKAP4 | Testis | 523 |
| VYADQVNI | HLA-A24:02 | AKAP4 | Testis | 524 |
| KSKCLHHSI | HLA-A30:01 | AKAP4 | Testis | 525 |
| RGTRCIHSGA | HLA-A30:01 | AKAP4 | Testis | 526 |
| GTRCIHSGA | HLA-A30:01 | AKAP4 | Testis | 527 |
| KTLKVHSSGK | HLA-A30:01 | AKAP4 | Testis | 528 |
| ASMSNRSDK | HLA-A30:01 | AKAP4 | Testis | 529 |
| KERDKGKMK | HLA-A30:01 | AKAP4 | Testis | 530 |
| GDKDGQLEK | HLA-A30:01 | AKAP4 | Testis | 531 |
| HTKEIVSDI | HLA-A30:01 | AKAP4 | Testis | 532 |
| RQPDEAVGK | HLA-A30:01 | AKAP4 | Testis | 533 |
| KEFADSISK | HLA-A30:01 | AKAP4 | Testis | 534 |
| EVMKFAKER | HLA-A33:03 | AKAP4 | Testis | 535 |
| MMSDDIDWLR | HLA-A33:03 | AKAP4 | Testis | 536 |
| DIDWLRSHR | HLA-A33:03 | AKAP4 | Testis | 537 |
| SVKQLESHR | HLA-A33:03 | AKAP4 | Testis | 538 |
| YLMNRPQNLR | HLA-A33:03 | AKAP4 | Testis | 539 |
| DDLSFYVNR | HLA-A33:03 | AKAP4 | Testis | 540 |
| VNIDYLMNR | HLA-A33:03 | AKAP4 | Testis | 541 |
| EAVGKVAR | HLA-A33:03 | AKAP4 | Testis | 542 |
| DFVSAVKR | HLA-A33:03 | AKAP4 | Testis | 543 |
| RPQNLRLEM | HLA-B07:02 | AKAP4 | Testis | 544 |
| SPRTPASKI | HLA-B07:02 | AKAP4 | Testis | 545 |
| KPIPASVVL | HLA-B07:02 | AKAP4 | Testis | 546 |
| APSDPVSVL | HLA-B07:02 | AKAP4 | Testis | 547 |
| INRPQNERLEM | HLA-B07:02 | AKAP4 | Testis | 548 |
| TPASKIASEM | HLA-B07:02 | AKAP4 | Testis | 549 |
| MLKRLVSAL | HLA-B08:01 | AKAP4 | Testis | 550 |
| YLMNRPQNL | HLA-B08:01 | AKAP4 | Testis | 551 |
| AMLKRLVSAL | HLA-B08:01 | AKAP4 | Testis | 552 |
| MEAMLKRLVSAL | HLA-B08:01 | AKAP4 | Testis | 553 |
| YVNRLSSL | HLA-B08:01 | AKAP4 | Testis | 554 |
| HDPKCRNQSL | HLA-B08:01 | AKAP4 | Testis | 555 |
| DPKCRNQSL | HLA-B08:01 | AKAP4 | Testis | 556 |
| EHILKEGL | HLA-B08:01 | AKAP4 | Testis | 557 |
| SDLQKYAL | HLA-B08:01 | AKAP4 | Testis | 558 |
| MAYEAVEL | HLA-B08:01 | AKAP4 | Testis | 559 |
| MDMSNIVLMLI | HLA-B13:02 | AKAP4 | Testis | 560 |
| MDMSNIVLML | HLA-B13:02 | AKAP4 | Testis | 561 |
| MSNIVLMLL | HLA-B13:02 | AKAP4 | Testis | 562 |
| MDMSNIVLM | HLA-B13:02 | AKAP4 | Testis | 563 |
| KMDMSNIVLMLI | HLA-B13:02 | AKAP4 | Testis | 564 |
| HILKEGLTI | HLA-B13:02 | AKAP4 | Testis | 565 |
| YQDSLGHEV | HLA-B13:02 | AKAP4 | Testis | 566 |
| GQQDQDRKV | HLA-B13:02 | AKAP4 | Testis | 567 |
| RSHRQVCKV | HLA-B13:02 | AKAP4 | Testis | 568 |
| GQSAKALSV | HLA-B13:02 | AKAP4 | Testis | 569 |
| MAYSDTTMM | HLA-B46:01 | AKAP4 | Testis | 570 |
| MMAYSDTTM | HLA-B46:01 | AKAP4 | Testis | 571 |
| YANQVASDM | HLA-B46:01 | AKAP4 | Testis | 572 |
| CSIDDLSFY | HLA-B46:01 | AKAP4 | Testis | 573 |
| YALGFQHAL | HLA-B46:01 | AKAP4 | Testis | 574 |
| VASDMMVSL | HLA-B46:01 | AKAP4 | Testis | 575 |
| LAKDLIVSA | HLA-B46:01 | AKAP4 | Testis | 576 |
| SGKPIPASV | HLA-B46:01 | AKAP4 | Testis | 577 |
| YSNDGAAL | HLA-B46:01 | AKAP4 | Testis | 578 |
| MMAYSDTTM | HLA-C01:02 | AKAP4 | Testis | 579 |
| YALGFQHAL | HLA-C01:02 | AKAP4 | Testis | 580 |
| YLMNRPQNL | HLA-C01:02 | AKAP4 | Testis | 581 |
| KMDMSNIVL | HLA-C01:02 | AKAP4 | Testis | 582 |
| VASDMMVSL | HLA-C01:02 | AKAP4 | Testis | 583 |
| ISPDGECSI | HLA-C01:02 | AKAP4 | Testis | 584 |
| QAPSDPVSVL | HLA-C01:02 | AKAP4 | Testis | 585 |
| QAPSDPVSV | HLA-C01:02 | AKAP4 | Testis | 586 |
| YSNDGAAL | HLA-C01:02 | AKAP4 | Testis | 587 |
| AMPQNYQDSL | HLA-C01:02 | AKAP4 | Testis | 588 |
| MAYSDTTMM | HLA-C03:04 | AKAP4 | Testis | 589 |
| YALGFQHAL | HLA-C03:04 | AKAP4 | Testis | 590 |
| VASDMMVSL | HLA-C03:04 | AKAP4 | Testis | 591 |
| YANQVASDM | HLA-C03:04 | AKAP4 | Testis | 592 |
| AASQFNVPM | HLA-C03:04 | AKAP4 | Testis | 593 |
| MAYEAVEL | HLA-C03:04 | AKAP4 | Testis | 594 |
| FADSISKGL | HLA-C03:04 | AKAP4 | Testis | 595 |
| YSNDGAAL | HLA-C03:04 | AKAP4 | Testis | 596 |
| YQDSLGHEV | HLA-C04:01 | AKAP4 | Testis | 597 |
| YLMNRPQNL | HLA-C04:01 | AKAP4 | Testis | 598 |
| ASDMMVSLM | HLA-C04:01 | AKAP4 | Testis | 599 |
| FADSISKGL | HLA-C04:01 | AKAP4 | Testis | 600 |
| SIDDLSFYV | HLA-C04:01 | AKAP4 | Testis | 601 |
| KMDMSNIVI | HLA-C04:01 | AKAP4 | Testis | 602 |
| ATDIMEAML | HLA-C04:01 | AKAP4 | Testis | 603 |
| QFNVPMLYF | HLA-C04:01 | AKAP4 | Testis | 604 |
| LLDWLLANL | HLA-C04:01 | AKAP4 | Testis | 605 |
| FVDVSTLNV | HLA-C04:01 | AKAP4 | Testis | 606 |
| KMDMSNIVLM | HLA-C04:01 | AKAP4 | Testis | 607 |
| FYVNRLSSL | HLA-C07:01 | AKAP4 | Testis | 608 |
| YLMNRPQNL | HLA-C07:01 | AKAP4 | Testis | 609 |
| MAYSDTTMM | HLA-C07:01 | AKAP4 | Testis | 610 |
| NRPQNLRLEM | HLA-C07:01 | AKAP4 | Testis | 611 |
| SQFNVPMLY | HLA-C07:01 | AKAP4 | Testis | 612 |
| CRNQSLEF | HLA-C07:01 | AKAP4 | Testis | 613 |
| HRAPGPSTC | HLA-C07:01 | AKAP4 | Testis | 614 |
| SREGGQKSF | HLA-C07:01 | AKAP4 | Testis | 615 |
| HRGVCKVDL | HLA-C07:01 | AKAP4 | Testis | 616 |
| APSDPVSVL | HLA-C07:01 | AKAP4 | Testis | 617 |
| FYVNRLSSL | HLA-C07:02 | AKAP4 | Testis | 618 |
| YLMNRPQNL | HLA-C07:02 | AKAP4 | Testis | 619 |
| SFYVNRLSSL | HLA-C07:02 | AKAP4 | Testis | 620 |
| MAYSDTTMM | HLA-C07:02 | AKAP4 | Testis | 621 |
| NRPQNLRLEM | HLA-C07:02 | AKAP4 | Testis | 622 |
| FYVNRLSSLV | HLA-C07:02 | AKAP4 | Testis | 623 |
| HRAPGPSTC | HLA-C07:02 | AKAP4 | Testis | 624 |
| NRPQNLRL | HLA-C07:02 | AKAP4 | Testis | 625 |
| KYSNDGAAL | HLA-C07:02 | AKAP4 | Testis | 626 |
| MTEAALLLL | HLA-A01:01 | ALPPL2 | Uterine | 627 |
| FLAMDRFPY | HLA-A01:01 | ALPPL2 | Uterine | 628 |
| WLAKHQGARY | HLA-A01:01 | ALPPL2 | Uterine | 629 |
| MTEAALLL | HLA-A01:01 | ALPPL2 | Uterine | 630 |
| RTELLQASL | HLA-A01:01 | ALPPL2 | Uterine | 631 |
| FLAMDRFPYV | HLA-A02:01 | ALPPL2 | Uterine | 632 |
| TFLAMDRFPYV | HLA-A02:01 | ALPPL2 | Uterine | 633 |
| ETFLAMDRFPYV | HLA-A02:01 | ALPPL2 | Uterine | 634 |
| FLAMDRFPYVA | HLA-A02:01 | ALPPL2 | Uterine | 635 |
| FLAMDRFPYVAL | HLA-A02:01 | ALPPL2 | Uterine | 636 |
| TFLAMDRFPYVA | HLA-A02:01 | ALPPL2 | Uterine | 637 |
| YVWNRTELL | HLA-A02:01 | ALPPL2 | Uterine | 638 |
| LLQASLDPSV | HLA-A02:01 | ALPPL2 | Uterine | 639 |
| SLMEMTEAALLL | HLA-A02:01 | ALPPL2 | Uterine | 640 |
| SLMEMTEAALL | HLA-A02:01 | ALPPL2 | Uterine | 641 |
| KLGPETFLA | HLA-A02:01 | ALPPL2 | Uterine | 642 |
| LLLLGTATA | HLA-A02:01 | ALPPL2 | Uterine | 643 |
| KLGPETFL | HLA-A02:01 | ALPPL2 | Uterine | 644 |
| ALSKTYSV | HLA-A02:01 | ALPPL2 | Uterine | 645 |
| ALSKTYSVDK | HLA-A03:01 | ALPPL2 | Uterine | 646 |
| GAYAHTVNR | HLA-A03:01 | ALPPL2 | Uterine | 647 |
| AGAYAHTVNR | HLA-A03:01 | ALPPL2 | Uterine | 648 |
| KLGPETFLAM | HLA-A03:01 | ALPPL2 | Uterine | 649 |
| ALLLLSRNPR | HLA-A03:01 | ALPPL2 | Uterine | 650 |
| GAYAHTVNR | HLA-A11:01 | ALPPL2 | Uterine | 651 |
| AGAYAHTVNR | HLA-A11:01 | ALPPL2 | Uterine | 652 |
| ALSKTYSVDK | HLA-A11:01 | ALPPL2 | Uterine | 653 |
| AALLLLSR | HLA-A11:01 | ALPPL2 | Uterine | 654 |
| EAALLLLSR | HLA-A11:01 | ALPPL2 | Uterine | 655 |
| RYVWNRTELL | HLA-A24:02 | ALPPL2 | Uterine | 656 |
| AYAHTVNRNW | HLA-A24:02 | ALPPL2 | Uterine | 657 |
| ARYVWNRTELL | HLA-A24:02 | ALPPL2 | Uterine | 658 |
| VWNRTELL | HLA-A24:02 | ALPPL2 | Uterine | 659 |
| TFLAMDRF | HLA-A24:02 | ALPPL2 | Uterine | 660 |
| LSKTYSVDK | HLA-A30:01 | ALPPL2 | Uterine | 661 |
| RVQHASPAGA | HLA-A30:01 | ALPPL2 | Uterine | 662 |
| GAYAHTVNR | HLA-A30:01 | ALPPL2 | Uterine | 663 |
| ALSKTYSVDK | HLA-A30:01 | ALPPL2 | Uterine | 664 |
| KTYSVDKHV | HLA-A30:01 | ALPPL2 | Uterine | 665 |
| AAHPGPSVV | HLA-A30:01 | ALPPL2 | Uterine | 666 |
| YTACDLAPR | HLA-A33:03 | ALPPL2 | Uterine | 667 |
| LLLLSRNPR | HLA-A33:03 | ALPPL2 | Uterine | 668 |
| GAYAHTVNR | HLA-A33:03 | ALPPL2 | Uterine | 669 |
| EAALLLLSR | HLA-A33:03 | ALPPL2 | Uterine | 670 |
| ETFLAMDR | HLA-A33:03 | ALPPL2 | Uterine | 671 |
| APRAGTTDAA | HLA-B07:02 | ALPPL2 | Uterine | 672 |
| APRAGTTDA | HLA-B07:02 | ALPPL2 | Uterine | 673 |
| HPGPSVVPAL | HLA-B07:02 | ALPPL2 | Uterine | 674 |
| GPSVVPALL | HLA-B07:02 | ALPPL2 | Uterine | 675 |
| FLAMDRFPYV | HLA-B08:01 | ALPPL2 | Uterine | 676 |
| FPYVALSKTYSV | HLA-B08:01 | ALPPL2 | Uterine | 677 |
| VALSKTYSV | HLA-B08:01 | ALPPL2 | Uterine | 678 |
| YVWNRTELL | HLA-B08:01 | ALPPL2 | Uterine | 679 |
| TELLQASL | HLA-B08:01 | ALPPL2 | Uterine | 680 |
| MEMTEAALLL | HLA-B13:02 | ALPPL2 | Uterine | 681 |
| MEMTEAALL | HLA-B13:02 | ALPPL2 | Uterine | 682 |
| MEMTEAALLLL | HLA-B13:02 | ALPPL2 | Uterine | 683 |
| RQAAEALGA | HLA-B13:02 | ALPPL2 | Uterine | 684 |
| ALSKTYSV | HLA-B13:02 | ALPPL2 | Uterine | 685 |
| AAHPQPSVV | HLA-B13:02 | ALPPL2 | Uterine | 686 |
| FLAMDRFPY | HLA-B46:01 | ALPPL2 | Uterine | 687 |
| FLAMDRFPYV | HLA-B46:01 | ALPPL2 | Uterine | 688 |
| YVWNRTELL | HLA-B46:01 | ALPPL2 | Uterine | 689 |
| AAHPGPSVV | HLA-B46:01 | ALPPL2 | Uterine | 690 |
| AAHPGPSV | HLA-B46:01 | ALPPL2 | Uterine | 691 |
| LAKHQGARY | HLA-B46:01 | ALPPL2 | Uterine | 692 |
| YVWNRTELL | HLA-C01:02 | ALPPL2 | Uterine | 693 |
| LGPETFLAM | HLA-C01:02 | ALPPL2 | Uterine | 694 |
| MTEAALLLL | HLA-C01:02 | ALPPL2 | Uterine | 695 |
| PGPSVVPAL | HLA-C01:02 | ALPPL2 | Uterine | 696 |
| LAPRAGTTD | HLA-C01:02 | ALPPL2 | Uterine | 697 |
| AAHPGPSVV | HLA-C03:04 | ALPPL2 | Uterine | 698 |
| YVWNRTELL | HLA-C03:04 | ALPPL2 | Uterine | 699 |
| FLAMDRFPYV | HLA-C03:04 | ALPPL2 | Uterine | 700 |
| AAHPGPSV | HLA-C03:04 | ALPPL2 | Uterine | 701 |
| DAAHPGPSV | HLA-C03:04 | ALPPL2 | Uterine | 702 |
| YVWNRTELL | HLA-C04:01 | ALPPL2 | Uterine | 703 |
| FLAMDRFPYV | HLA-C04:01 | ALPPL2 | Uterine | 704 |
| MTEAALLLL | HLA-C04:01 | ALPPL2 | Uterine | 705 |
| VWNRTELL | HLA-C04:01 | ALPPL2 | Uterine | 706 |
| KLGPETFL | HLA-C04:01 | ALPPL2 | Uterine | 707 |
| YVWNRTELL | HLA-C07:01 | ALPPL2 | Uterine | 708 |
| FLAMDRFPYV | HLA-C07:01 | ALPPL2 | Uterine | 709 |
| ARYVWNRTELL | HLA-C07:01 | ALPPL2 | Uterine | 710 |
| HPGPSVVP | HLA-C07:01 | ALPPL2 | Uterine | 711 |
| YSVDKHVP | HLA-C07:01 | ALPPL2 | Uterine | 712 |
| HPGPSVVPA | HLA-C07:01 | ALPPL2 | Uterine | 713 |
| YVWNRTELL | HLA-C07:02 | ALPPL2 | Uterine | 714 |
| RYVWNRTELL | HLA-C07:02 | ALPPL2 | Uterine | 715 |
| FLAMDRFPYV | HLA-C07:02 | ALPPL2 | Uterine | 716 |
| NRTELLQASL | HLA-C07:02 | ALPPL2 | Uterine | 717 |
| LGPETFLAM | HLA-C07:02 | ALPPL2 | Uterine | 718 |
| PGPSVVPAL | HLA-C07:02 | ALPPL2 | Uterine | 719 |
| VSPPNENVAIY | HLA-A01:01 | AMY2A | Pancreas | 720 |
| NNDDWSFSL | HLA-A01:01 | AMY2A | Pancreas | 721 |
| FNNDDWSFSL | HLA-A01:01 | AMY2A | Pancreas | 722 |
| LIGLLDLAL | HLA-A01:01 | AMY2A | Pancreas | 723 |
| YVDAVINHMC | HLA-A01:01 | AMY2A | Pancreas | 724 |
| RLTGLLDLAL | HLA-A02:01 | AMY2A | Pancreas | 725 |
| IVFNNDDWSPSL | HLA-A02:01 | AMY2A | Pancreas | 726 |
| RLTGLLDLA | HLA-A02:01 | AMY2A | Pancreas | 727 |
| YLKNWGEGWGFV | HLA-A02:01 | AMY2A | Pancreas | 728 |
| FVPSDRALVFV | HLA-A02:01 | AMY2A | Pancreas | 729 |
| FVPSDRALV | HLA-A02:01 | AMY2A | Pancreas | 730 |
| AVINHMCGNAV | HLA-A02:01 | AMY2A | Pancreas | 731 |
| AVINHMQGNA | HLA-A02:01 | AMY2A | Pancreas | 732 |
| NMVIFRNVV | HLA-A02:01 | AMY2A | Pancreas | 733 |
| HMCGNAVSA | HLA-A02:01 | AMY2A | Pancreas | 734 |
| RLTGLLDL | HLA-A02:01 | AMY2A | Pancreas | 735 |
| VPSDRALVFV | HLA-A02:01 | AMY2A | Pancreas | 736 |
| FNNDDWSFSL | HLA-A02:01 | AMY2A | Pancreas | 737 |
| NNDDWSFSL | HLA-A02:01 | AMY2A | Pancreas | 738 |
| NWGEGWGFV | HLA-A02:01 | AMY2A | Pancreas | 739 |
| RQIRNMVIFR | HLA-A03:01 | AMY2A | Pancreas | 740 |
| TGLLDLALEK | HLA-A03:01 | AMY2A | Pancreas | 141 |
| RLTGLLDLALEK | HLA-A03:01 | AMY2A | Pancreas | 742 |
| AIYNPFRPW | HLA-A03:01 | AMY2A | Pancreas | 743 |
| QIRNMVIFR | HLA-A03:01 | AMY2A | Pancreas | 744 |
| AIYNPFRPWW | HLA-A03:01 | AMY2A | Pancreas | 745 |
| TQLLDLALEK | HLA-A11:01 | AMY2A | Pancreas | 746 |
| RQIRNMVIFR | HLA-A11:01 | AMY2A | Pancreas | 747 |
| LTGLLDLALEK | HLA-A11:01 | AMY2A | Pancreas | 748 |
| ATYNPFRPWWER | HLA-A11:01 | AMY2A | Pancreas | 749 |
| AIYNPFRPW | HLA-A11:01 | AMY2A | Pancreas | 750 |
| QVIDLGGEPIK | HLA-A11:01 | AMY2A | Pancreas | 751 |
| NVAIYNPFR | HLA-A11:01 | AMY2A | Pancreas | 752 |
| TYNPFRPWW | HLA-A24:02 | AMY2A | Pancreas | 753 |
| VFNNDDWSF | HLA-A24:02 | AMY2A | Pancreas | 754 |
| RQIRNMVIF | HLA-A24:02 | AMY2A | Pancreas | 755 |
| NYNDATQVI | HLA-A24:02 | AMY2A | Pancreas | 756 |
| RWRQIRNMVI | HLA-A30:01 | AMY2A | Pancreas | 757 |
| RQIRNMVIFR | HLA-A30:01 | AMY2A | Pancreas | 758 |
| QVRDCRLTG | HLA-A30:01 | AMY2A | Pancreas | 759 |
| RNMVIFRNV | HLA-A30:01 | AMY2A | Pancreas | 760 |
| AIYNPFRPW | HLA-A30:01 | AMY2A | Pancreas | 761 |
| WSFSLTLQT | HLA-A30:01 | AMY2A | Pancreas | 762 |
| QIRNMVIFR | HLA-A33:03 | AMY2A | Pancreas | 763 |
| NVAIYNPFR | HLA-A33:03 | AMY2A | Pancreas | 764 |
| YNPFRPWWER | HLA-A33:03 | AMY2A | Pancreas | 765 |
| YNPFRPWWER | HLA-A33:03 | AMY2A | Pancreas | 766 |
| VPSDRALVF | HLA-B07:02 | AMY2A | Pancreas | 767 |
| FVPSDRALVF | HLA-B07:02 | AMY2A | Pancreas | 768 |
| QVRDCRLTGL | HLA-B07:02 | AMY2A | Pancreas | 769 |
| VPSDRALV | HLA-B07:02 | AMY2A | Pancreas | 770 |
| VPSDRALVFV | HLA-B07:02 | AMY2A | Pancreas | 771 |
| WRQIRNMVI | HLA-B08:01 | AMY2A | Pancreas | 772 |
| WRQIRNMVIF | HLA-B08:01 | AMY2A | Pancreas | 773 |
| QVRDCRLTGL | HLA-B08:01 | AMY2A | Pancreas | 774 |
| DATQVIDI | HLA-B08:01 | AMY2A | Pancreas | 775 |
| VPSDRALVF | HLA-B08:01 | AMY2A | Pancreas | 776 |
| TGLLDLAL | HLA-B08:01 | AMY2A | Pancreas | 777 |
| RNMVIFRNV | HLA-B13:02 | AMY2A | Pancreas | 778 |
| FNNDDWSFSLTL | HLA-B13:02 | AMY2A | Pancreas | 779 |
| NDDWSFSLTL | HLA-B13:02 | AMY2A | Pancreas | 780 |
| RQIRNMVI | HLA-B13:02 | AMY2A | Pancreas | 781 |
| RQIRNMVIF | HLA-B13:02 | AMY2A | Pancreas | 782 |
| RLTGLLDL | HLA-B13:02 | AMY2A | Pancreas | 783 |
| FVPSDRALVF | HLA-B46:01 | AMY2A | Pancreas | 784 |
| RQIRNMVIF | HLA-B46:01 | AMY2A | Pancreas | 785 |
| AIYNPFRPW | HLA-B46:01 | AMY2A | Pancreas | 786 |
| VFNNDDWSF | HLA-B46:01 | AMY2A | Pancreas | 787 |
| FNNDDWSF | HLA-B46:01 | AMY2A | Pancreas | 788 |
| FVPSDRALV | HLA-C01:02 | AMY2A | Pancreas | 789 |
| FVPSDRALVF | HLA-C01:02 | AMY2A | Pancreas | 790 |
| LTGLLDLAL | HLA-C01:02 | AMY2A | Pancreas | 791 |
| FVPSDRAL | HLA-C01:02 | AMY2A | Pancreas | 792 |
| FVPSDRALVF | HLA-C03:04 | AMY2A | Pancreas | 793 |
| VPSDRALVF | HLA-C03:04 | AMY2A | Pancreas | 794 |
| FVPSDRALV | HLA-C03:04 | AMY2A | Pancreas | 795 |
| AIYNPFRPW | HLA-C03:04 | AMY2A | Pancreas | 796 |
| FVPSDRAL | HLA-C03:04 | AMY2A | Pancreas | 797 |
| GFVPSDRAL | HLA-C03:04 | AMY2A | Pancreas | 798 |
| NYNDATQVI | HLA-C04:01 | AMY2A | Pancreas | 799 |
| FVPSDRALVF | HLA-C04:01 | AMY2A | Pancreas | 800 |
| WRQIRNMVI | HLA-C04:01 | AMY2A | Pancreas | 801 |
| VFNNDDWSF | HLA-C04:01 | AMY2A | Pancreas | 802 |
| NNDDWSFSL | HLA-C04:01 | AMY2A | Pancreas | 803 |
| WRQIRNMVI | HLA-C07:01 | AMY2A | Pancreas | 804 |
| CRLTGLLDL | HLA-C07:01 | AMY2A | Pancreas | 805 |
| NYNDATQVI | HLA-C07:01 | AMY2A | Pancreas | 806 |
| IRNMVIFRN | HLA-C07:01 | AMY2A | Pancreas | 807 |
| CRLTGLLD | HLA-C07:01 | AMY2A | Pancreas | 808 |
| NNDDWSFSL | HLA-C07:01 | AMY2A | Pancreas | 809 |
| WRQIRNMVI | HLA-C07:02 | AMY2A | Pancreas | 810 |
| CRLTQLLDL | HLA-C07:02 | AMY2A | Pancreas | 811 |
| NYNDATQVI | HLA-C07:02 | AMY2A | Pancreas | 812 |
| FVPSDRAL | HLA-C07:02 | AMY2A | Pancreas | 813 |
| VPSDRALVF | HLA-C07:02 | AMY2A | Pancreas | 814 |
| MNVDVSSTIY | HLA-A01:01 | ANKRD30A | Breast | 815 |
| NVDVSSTIY | HLA-A01:01 | ANKRD30A | Breast | 816 |
| LVDVYGNTALHY | HLA-A01:01 | ANKRD30A | Breast | 817 |
| KMNVDVSSTIY | HLA-A01:01 | ANKRD30A | Breast | 818 |
| GADINLVDVY | HLA-A01:01 | ANKRD30A | Breast | 819 |
| RKMNVDVSSTIY | HLA-A01:01 | ANKRD30A | Breast | 820 |
| PSESKQKDY | HLA-A01:01 | ANKRD30A | Breast | 821 |
| TIDIHFLER | HLA-A01:01 | ANKRD30A | Breast | 822 |
| YSCDSRSLF | HLA-A01:01 | ANKRD30A | Breast | 823 |
| YLLHENCML | HLA-A02:01 | ANKRD30A | Breast | 824 |
| AVYSEILSV | HLA-A02:01 | ANKRD30A | Breast | 825 |
| SLSKILDTV | HLA-A02:01 | ANKRD30A | Breast | 826 |
| SLDQKLFQL | HLA-A02:01 | ANKRD30A | Breast | 827 |
| SLIPLLLSI | HLA-A02:01 | ANKRD30A | Breast | 828 |
| YAVYSEILSV | HLA-A02:01 | ANKRD30A | Breast | 829 |
| NMWLQQQLV | HLA-A02:01 | ANKRD30A | Breast | 830 |
| LLSHGAVIEV | HLA-A02:01 | ANKRD30A | Breast | 831 |
| SLFESSAKI | HLA-A02:01 | ANKRD30A | Breast | 832 |
| KLLSHGAVIEV | HLA-A02:01 | ANKRD30A | Breast | 833 |
| FLKAPCRMKV | HLA-A02:01 | ANKRD30A | Breast | 834 |
| FLLIKNANA | HLA-A02:01 | ANKRD30A | Breast | 835 |
| AVYSEILSVV | HLA-A02:01 | ANKRD30A | Breast | 836 |
| ASLTPLLLSI | HLA-A02:01 | ANKRD30A | Breast | 837 |
| ILIDSGADINLV | HLA-A02:01 | ANKRD30A | Breast | 838 |
| VLIAENTML | HLA-A02:01 | ANKRD30A | Breast | 839 |
| ILIDSGADINL | HLA-A02:01 | ANKRD30A | Breast | 840 |
| SLFESSAKIQV | HLA-A02:01 | ANKRD30A | Breast | 841 |
| ILKEKNAEL | HLA-A02:01 | ANKRD30A | Breast | 842 |
| ILIDSGADI | HLA-A02:01 | ANKRD30A | Breast | 843 |
| ILNEKIREEL | HLA-A02:01 | ANKRD30A | Breast | 844 |
| IMEYIRKLSK | HLA-A03:01 | ANKRD30A | Breast | 845 |
| KMQHHLLKEK | HLA-A03:01 | ANKRD30A | Breast | 846 |
| RMKVSIPTK | HLA-A03:01 | ANKRD30A | Breast | 847 |
| LELMDMQTFK | HLA-A03:01 | ANKRD30A | Breast | 848 |
| LLHENCMLKK | HLA-A03:01 | ANKRD30A | Breast | 849 |
| KALELMDMQTFK | HLA-A03:01 | ANKRD30A | Breast | 850 |
| KVLEKGRSK | HLA-A03:01 | ANKRD30A | Breast | 851 |
| ELMDMQTFK | HLA-A03:01 | ANKRD30A | Breast | 852 |
| SLCETVSQK | HLA-A03:01 | ANKRD30A | Breast | 853 |
| RIYQYEKEK | HLA-A03:01 | ANKRD30A | Breast | 854 |
| KLEDSTSLSK | HLA-A03:01 | ANKRD30A | Breast | 855 |
| AVYSEILSVVAK | HLA-A03:01 | ANKRD30A | Breast | 856 |
| CVARVTSNK | HLA-A11:01 | ANKRD30A | Breast | 857 |
| RSLFESSAK | HLA-A11:01 | ANKRD30A | Breast | 858 |
| LELMDMQTFK | HLA-A11:01 | ANKRD30A | Breast | 859 |
| SVPNKAFELK | HLA-A11:01 | ANKRD30A | Breast | 860 |
| ATLKHQYQEK | HLA-A11:01 | ANKRD30A | Breast | 861 |
| ITIDIHFLERK | HLA-A11:01 | ANKRD30A | Breast | 862 |
| VCIPESIYQK | HLA-A11:01 | ANKRD30A | Breast | 863 |
| ITIDIHFLER | HLA-A11:01 | ANKRD30A | Breast | 864 |
| AVIEVHNK | HLA-A11:01 | ANKRD30A | Breast | 865 |
| QVCIPESIYQK | HLA-A11:01 | ANKRD30A | Breast | 866 |
| AVYSEILSVVAK | HLA-A11:01 | ANKRD30A | Breast | 867 |
| CIPESIYQK | HLA-A11:01 | ANKRD30A | Breast | 868 |
| KYKCTALML | HLA-A24:02 | ANKRD30A | Breast | 869 |
| VYSEILSVV | HLA-A24:02 | ANKRD30A | Breast | 870 |
| HYAVYSEIL | HLA-A24:02 | ANKRD30A | Breast | 871 |
| IYQKVMEI | HLA-A24:02 | ANKRD30A | Breast | 872 |
| QYSGQLKVLI | HLA-A24:02 | ANKRD30A | Breast | 873 |
| HYAVTCGFHHI | HLA-A24:02 | ANKRD30A | Breast | 874 |
| QYQEKENKYF | HLA-A24:02 | ANKRD30A | Breast | 875 |
| EYSCDSRSLF | HLA-A24:02 | ANKRD30A | Breast | 876 |
| QYSGQLKVL | HLA-A24:02 | ANKRD30A | Breast | 877 |
| RMKVSIPTK | HLA-A30:01 | ANKRD30A | Breast | 878 |
| RSKMIACPTK | HLA-A30:01 | ANKRD30A | Breast | 879 |
| GMKVSIPTK | HLA-A30:01 | ANKRD30A | Breast | 880 |
| RSKMIACPT | HLA-A30:01 | ANKRD30A | Breast | 881 |
| AQRKSKSLK | HLA-A30:01 | ANKRD30A | Breast | 882 |
| KSKSLKINL | HLA-A30:01 | ANKRD30A | Breast | 883 |
| KLKEESLTK | HLA-A30:01 | ANKRD30A | Breast | 884 |
| SQKDVCLPK | HLA-A30:01 | ANKRD30A | Breast | 885 |
| SQKDVCVPK | HLA-A30:01 | ANKRD30A | Breast | 886 |
| SLRETVSQK | HLA-A30:01 | ANKRD30A | Breast | 887 |
| KVLEKGRSK | HLA-A30:01 | ANKRD30A | Breast | 888 |
| ITIDIHFLER | HLA-A33:03 | ANKRD30A | Breast | 889 |
| FTWAAKGRPR | HLA-A33:03 | ANKRD30A | Breast | 890 |
| FTWPAKGRPR | HLA-A33:03 | ANKRD30A | Breast | 891 |
| NSWDSESLR | HLA-A33:03 | ANKRD30A | Breast | 892 |
| TVHSCERAR | HLA-A33:03 | ANKRD30A | Breast | 893 |
| NTLVSEHAQR | HLA-A33:03 | ANKRD30A | Breast | 894 |
| NYNNHLKNR | HLA-A33:03 | ANKRD30A | Breast | 895 |
| DEAAPLVER | HLA-A33:03 | ANKRD30A | Breast | 896 |
| EAAPLVER | HLA-A33:03 | ANKRD30A | Breast | 897 |
| IFNYNNHLKNR | HLA-A33:03 | ANKRD30A | Breast | 898 |
| FNYNNHLKNR | HLA-A33:03 | ANKRD30A | Breast | 899 |
| TPREITSPA | HLA-B07:02 | ANKRD30A | Breast | 900 |
| KPSAFKPAI | HLA-B07:02 | ANKRD30A | Breast | 901 |
| HPRLASAV | HLA-B07:02 | ANKRD30A | Breast | 902 |
| TPREIMSPA | HLA-B07:02 | ANKRD30A | Breast | 903 |
| WPAKGRPRKI | HLA-B07:02 | ANKRD30A | Breast | 904 |
| VPKATHQKEM | HLA-B07:02 | ANKRD30A | Breast | 905 |
| KPSAFEPAI | HLA-B07:02 | ANKRD30A | Breast | 906 |
| TPDEAASL | HLA-B07:02 | ANKRD30A | Breast | 907 |
| VPNKALEL | HLA-B07:02 | ANKRD30A | Breast | 908 |
| EPPGKPSAP | HLA-B07:02 | ANKRD30A | Breast | 909 |
| QMKKKFCVL | HLA-B08:01 | ANKRD30A | Breast | 910 |
| MEQMKKKFCVL | HLA-B08:01 | ANKRD30A | Breast | 911 |
| KMEQMKKKPCVL | HLA-B08:01 | ANKRD30A | Breast | 912 |
| MKKKFCVL | HLA-B08:01 | ANKRD30A | Breast | 913 |
| EQMKKKFCVL | HLA-B08:01 | ANKRD30A | Breast | 914 |
| MLKLEIATL | HLA-B08:01 | ANKRD30A | Breast | 915 |
| MEQMKKKFCVLK | HLA-B08:01 | ANKRD30A | Breast | 916 |
| TLKLKEESL | HLA-B08:01 | ANKRD30A | Breast | 917 |
| ILKEKNAEL | HLA-B08:01 | ANKRD30A | Breast | 918 |
| VNKYKCTAL | HLA-B08:01 | ANKRD30A | Breast | 919 |
| EVHNKASL | HLA-B08:01 | ANKRD30A | Breast | 920 |
| EAQRKSKSL | HLA-B08:01 | ANKRD30A | Breast | 921 |
| SEQIVEFLLI | HLA-B13:02 | ANKRD30A | Breast | 922 |
| KEILAMLKLEI | HLA-B13:02 | ANKRD30A | Breast | 923 |
| QEACANILI | HLA-B13:02 | ANKRD30A | Breast | 924 |
| HEEVVTFLV | HLA-B13:02 | ANKRD30A | Breast | 925 |
| HQEACANILI | HLA-B13:02 | ANKRD30A | Breast | 926 |
| SQYSGQLKV | HLA-B13:02 | ANKRD30A | Breast | 927 |
| EQIVEFLLI | HLA-B13:02 | ANKRD30A | Breast | 928 |
| AVYSEILSV | HLA-B13:02 | ANKRD30A | Breast | 929 |
| EQHRKELEV | HLA-B13:02 | ANKRD30A | Breast | 930 |
| KQDKEILEA | HLA-B13:02 | ANKRD30A | Breast | 931 |
| SQLENQKV | HLA-B13:02 | ANKRD30A | Breast | 932 |
| SAFEPATEM | HLA-B46:01 | ANKRD30A | Breast | 933 |
| SAFEPAIEM | HLA-B46:01 | ANKRD30A | Breast | 934 |
| YSCDSRSLF | HLA-B46:01 | ANKRD30A | Breast | 935 |
| HIHEQIMEY | HLA-B46:01 | ANKRD30A | Breast | 936 |
| SAFKPAIEM | HLA-B46:01 | ANKRD30A | Breast | 937 |
| YAVYSEILSV | HLA-B46:01 | ANKRD30A | Breast | 938 |
| ILKEKNAEL | HLA-B46:01 | ANKRD30A | Breast | 939 |
| HLKKRIYQY | HLA-B46:01 | ANKRD30A | Breast | 940 |
| SVPNKALEL | HLA-C01:02 | ANKRD30A | Breast | 941 |
| SIPTKALEL | HLA-C01:02 | ANKRD30A | Breast | 942 |
| SVPNKAFEL | HLA-C01:02 | ANKRD30A | Breast | 943 |
| KTPDEAASL | HLA-C01:02 | ANKRD30A | Breast | 944 |
| KTPDEAAPL | HLA-C01:02 | ANKRD30A | Breast | 945 |
| SAFEPAIEM | HLA-C01:02 | ANKRD30A | Breast | 946 |
| HSCERAREL | HLA-C01:02 | ANKRD30A | Breast | 947 |
| SAFEPATEM | HLA-C03:04 | ANKRD30A | Breast | 948 |
| SAFEPAIEM | HLA-C03:04 | ANKRD30A | Breast | 949 |
| SAFKPAIEM | HLA-C03:04 | ANKRD30A | Breast | 950 |
| RASQYSGQL | HLA-C03:04 | ANKRD30A | Breast | 951 |
| STIYNNEVL | HLA-C03:04 | ANKRD30A | Breast | 952 |
| YAVYSEILSV | HLA-C03:04 | ANKRD30A | Breast | 953 |
| VSIPTKALEL | HLA-C03:04 | ANKRD30A | Breast | 954 |
| VSIPTKAL | HLA-C03:04 | ANKRD30A | Breast | 955 |
| YSCDSRSI | HLA-C03:04 | ANKRD30A | Breast | 956 |
| HQEACANIL | HLA-C04:01 | ANKRD30A | Breast | 957 |
| GHEEVVTFL | HLA-C04:01 | ANKRD30A | Breast | 958 |
| YFEDIKILK | HLA-C04:01 | ANKRD30A | Breast | 959 |
| FHIAGDACL | HLA-C04:01 | ANKRD30A | Breast | 960 |
| SVPNKAFEL | HLA-C04:01 | ANKRD30A | Breast | 961 |
| HYAVYSEIL | HLA-C04:01 | ANKRD30A | Breast | 962 |
| AFEPAIEM | HLA-C04:01 | ANKRD30A | Breast | 963 |
| AFEPATEM | HLA-C04:01 | ANKRD30A | Breast | 964 |
| IFNYNNHL | HLA-C04:01 | ANKRD30A | Breast | 965 |
| TPDEAASL | HLA-C04:01 | ANKRD30A | Breast | 966 |
| SWDTESLC | HLA-C04:01 | ANKRD30A | Breast | 967 |
| SWDSESLC | HLA-C04:01 | ANKRD30A | Breast | 968 |
| HRTPLMKAL | HLA-C07:01 | ANKRD30A | Breast | 969 |
| SIYQKVMEI | HLA-C07:01 | ANKRD30A | Breast | 970 |
| KRSEQIVEF | HLA-C07:01 | ANKRD30A | Breast | 971 |
| FHHIHEQIM | HLA-C07:01 | ANKRD30A | Breast | 972 |
| ERKMQHHLL | HLA-C07:01 | ANKRD30A | Breast | 973 |
| NKYKCTALM | HLA-C07:01 | ANKRD30A | Breast | 974 |
| RRNADILNE | HLA-C07:01 | ANKRD30A | Breast | 975 |
| KRSEQIVE | HLA-C07:01 | ANKRD30A | Breast | 976 |
| CGFHHIHEQ | HLA-C07:01 | ANKRD30A | Breast | 977 |
| RSEQIVEF | HLA-C07:01 | ANKRD30A | Breast | 978 |
| HIHEQIMEY | HLA-C07:01 | ANKRD30A | Breast | 979 |
| HRTPLMKAL | HLA-C07:02 | ANKRD30A | Breast | 980 |
| KRSEQIVEF | HLA-C07:02 | ANKRD30A | Breast | 981 |
| HYAVYSEIL | HLA-C07:02 | ANKRD30A | Breast | 982 |
| FHHIHEQIM | HLA-C07:02 | ANKRD30A | Breast | 983 |
| KYKCTALML | HLA-C07:02 | ANKRD30A | Breast | 984 |
| SIYQKVMEI | HLA-C07:02 | ANKRD30A | Breast | 985 |
| FHIAGDACL | HLA-C07:02 | ANKRD30A | Breast | 986 |
| HIHEQIMEY | HLA-C07:02 | ANKRD30A | Breast | 987 |
| KTPDEAASL | HLA-C07:02 | ANKRD30A | Breast | 988 |
| CSGHTLLEY | HLA-A01:01 | AQP12B; | Pancreas | 989 |
| | | AQP12A | | |
| FACSGHTLLEY | HLA-A01:01 | AQP12B; | Pancreas | 990 |
| | | AQP12A | | |
| HLFQRNLFY | HLA-A01:01 | AQP12B; | Pancreas | 991 |
| | | AQP12A | | |
| LSDLHLLQS | HLA-A01:01 | AQP12B; | Pancreas | 992 |
| | | AQP12A | | |
| LSDLHLLQ | HLA-A01:01 | AQP12B; | Pancreas | 993 |
| | | AQP12A | | |
| LLEYVQVY | HLA-A01:01 | AQP12B; | Pancreas | 994 |
| | | AQP12A | | |
| FLLFLAHGV | HLA-A02:01 | AQP12B; | Pancreas | 995 |
| | | AQP12A | | |
| LMRLCWAWEL | HLA-A02:01 | AQP12A | Pancreas | 996 |
| LLLTLLFLL | HLA-A02:01 | AQP12B; | Pancreas | 997 |
| | | AQP12A | | |
| ALLPVGAYEV | HLA-A02:01 | AQP12B; | Pancreas | 998 |
| | | AQP12A | | |
| LLPVGAYEV | HLA-A02:01 | AQP12B; | Pancreas | 999 |
| | | AQP12A | | |
| LFLLFLAHGV | HLA-A02:01 | AQP12B; | Pancreas | 1000 |
| | | AQP12A | | |
| FLLFLAHGVT | HLA-A02:01 | AQP12B; | Pancreas | 1001 |
| | | AQP12A | | |
| TLMRLCWAWEL | HLA-A02:01 | AQP12A | Pancreas | 1002 |
| SLSFFFATFA | HLA-A02:01 | AQP12A | Pancreas | 1003 |
| FTSAFFNPA | HLA-A02:01 | AQP12B; | Pancreas | 1004 |
| | | AQP12A | | |
| TLLEYVQVYWL | HLA-A02:01 | AQP12B; | Pancreas | 1005 |
| | | AQP12A | | |
| SLPGTLLKL | HLA-A02:01 | AQP12B; | Pancreas | 1006 |
| | | AQP12A | | |
| TLLEYVQV | HLA-A02:01 | AQP12B; | Pancreas | 1007 |
| | | AQP12A | | |
| ALAASVTFA | HLA-A02:01 | AQP12B; | Pancreas | 1008 |
| | | AQP12A | | |
| TLVELGPWA | HLA-A02:01 | AQP12B; | Pancreas | 1009 |
| | | AQP12A | | |
| FLAHGVTL | HLA-A02:01 | AQP12B; | Pancreas | 1010 |
| | | AQP12A | | |
| HLFQRNLFY | HLA-A03:01 | AQP12B; | Pancreas | 1011 |
| | | AQP12A | | |
| RNLFYGQKNK | HLA-A03:01 | AQP12B; | Pancreas | 1012 |
| | | AQP12A | | |
| HLFQRNLFYGQK | HLA-A03:01 | AQP12B | Pancreas | 1013 |
| | | AQP12A | | |
| ALLVTVTAY | HLA-A03:01 | AQP12B; | Pancreas | 1014 |
| | | AQP12A | | |
| QSLPGTLLK | HLA-A03:01 | AQP12A | Pancreas | 1015 |
| QSLPGTLLK | HLA-A11:01 | AQP12A | Pancreas | 1016 |
| MAEQSLPGTLLK | HLA-A11:01 | AQP12A | Pancreas | 1017 |
| HLFQRNLFY | HLA-A11:01 | AQP12B; | Pancreas | 1018 |
| | | AQP12A | | |
| ATFALCEAAR | HLA-A11:01 | AQP12A | Pancreas | 1019 |
| GRLPHLFQR | HLA-A11:01 | AQP12B; | Pancreas | 1020 |
| | | AQP12A | | |
| AYTAGPFTSAFF | HLA-A24:02 | AQP12B; | Pancreas | 1021 |
| | | AQP12A | | |
| SLSFFFATF | HLA-A24:02 | AQP12B; | Pancreas | 1022 |
| | | AQP12A | | |
| SFFFATFAL | HLA-A24:02 | AQP12A | Pancreas | 1023 |
| AYSGPAVALL | HLA-A24:02 | AQP12B; | Pancreas | 1024 |
| | | AQP12A | | |
| AYSGPAVAL | HLA-A24:02 | AQP12B; | Pancreas | 1025 |
| | | AQP12A | | |
| LLTLLFLLF | HLA-A24:02 | AQP12B; | Pancreas | 1026 |
| | | AQP12A | | |
| KYRAPRGKPA | HLA-A30:01 | AQP12B; | Pancreas | 1027 |
| | | AQP12A | | |
| ASKALLPVGA | HLA-A30:01 | AQP12B; | Pancreas | 1028 |
| | | AQP12A | | |
| RAPRQKPAPA | HLA-A30:01 | AQP12B; | Pancreas | 1029 |
| | | AQP12A | | |
| KNKYRAPRQK | HLA-A30:01 | AQP12B: | Pancreas | 1030 |
| | | AQP12A | | |
| HTLLEYVQV | HLA-A30:01 | AQP12B; | Pancreas | 1031 |
| | | AQP12A | | |
| SVREPGRSGV | HLA-A30:01 | AQP12B; | Pancreas | 1032 |
| | | AQP12A | | |
| EVFAREAMR | HLA-A33:03 | AQP12A | Pancreas | 1033 |
| YEVFAREAMR | HLA-A33:03 | AQP12A | Pancreas | 1034 |
| TFALCEAAR | HLA-A33:03 | AQP12A | Pancreas | 1035 |
| LFYGQKNKYR | HLA-A33:03 | AQP12B; | Pancreas | 1036 |
| | | AQP12A | | |
| MAQSCSSALR | HLA-A33:03 | AQP12B; | Pancreas | 1037 |
| | | AQP12A | | |
| FYGQKNKYR | HLA-A33:03 | AQP12B; | Pancreas | 1038 |
| | | AQP12A | | |
| AARRASKAL | HLA-B07:02 | AQP12B; | Pancreas | 1039 |
| | | AQP12A | | |
| APRGKPAPA | HLA-B07:02 | AQP12B; | Pancreas | 1040 |
| | | AQP12A | | |
| RAPRGKPAPA | HLA-B07:02 | AQP12B; | Pancreas | 1041 |
| | | AQP12A | | |
| GPDLLLTLL | HLA-B07:02 | AQP12B; | Pancreas | 1042 |
| | | AQP12A | | |
| AYSGPAVAL | HLA-B07:02 | AQP12B; | Pancreas | 1043 |
| | | AQP12A | | |
| LLHLRHSPPA | HLA-B08:01 | AQP12B; | Pancreas | 1044 |
| | | AQP12A | | |
| LLHLRHSPP | HLA-B08:01 | AQP12B; | Pancreas | 1045 |
| | | AQP12A | | |
| LMRLCWAWEL | HLA-B08:01 | AQP12A | Pancreas | 1046 |
| LLHQGRLPHL | HLA-B08:01 | AQP12B; | Pancreas | 1047 |
| | | AQP12A | | |
| DLHLLQSL | HLA-B08:01 | AQP12B; | Pancreas | 1048 |
| | | AQP12A | | |
| SANPTVSL | HLA-B08:01 | AQP12B; | Pancreas | 1049 |
| | | AQP12A | | |
| EMRTLVEL | HLA-B08:01 | AQP12B; | Pancreas | 1050 |
| | | AQP12A | | |
| LEMRTLVEL | HLA-B13:02 | AQP12B | Pancreas | 1051 |
| | | AQP12A | | |
| WELSDLHLL | HLA-B13:02 | AQP12B; | Pancreas | 1052 |
| | | AQP12A | | |
| LEYVQVYWL | HLA-B13:02 | AQP12B; | Pancreas | 1053 |
| | | AQP12A | | |
| SLPGTLLKL | HLA-B13:02 | AQP12B: | Pancreas | 1054 |
| | | AQP12A | | |
| RLPHLFQRN | HLA-B13:02 | AQP12B; | Pancreas | 1055 |
| | | AQP12A | | |
| ALLPVGAYEV | HLA-B13:02 | AQP12B; | Pancreas | 1056 |
| | | AQP12A | | |
| YTAGPFTSAF | HLA-B46:01 | AQP12B; | Pancreas | 1057 |
| | | AQP12A | | |
| FAREAMRTL | HLA-B46:01 | AQP12A | Pancreas | 1058 |
| FAREAVGAV | HLA-B46:01 | AQP12B; | Pancreas | 1059 |
| | | AQP12A | | |
| TAGPFTSAF | HLA-B46:01 | AQP12B; | Pancreas | 1060 |
| | | AQP12A | | |
| ASANPTVSL | HLA-B46:01 | AQP12B; | Pancreas | 1061 |
| | | AQP12A | | |
| SANPTVSL | HLA-B46:01 | AQP12B; | Pancreas | 1062 |
| | | AQP12A | | |
| YSGPAVALL | HLA-C01:02 | AQP12B; | Pancreas | 1063 |
| | | AQP12A | | |
| MAQSCSSAL | HLA-C01:02 | AQP12B; | Pancreas | 1064 |
| | | AQP12A | | |
| FGPDLLLTL | HLA-C01:02 | AQP12B; | Pancreas | 1065 |
| | | AQP12A | | |
| LGPLTGMVL | HLA-C01:02 | AQP12B; | Pancreas | 1066 |
| | | AQP12A | | |
| SLPGTLLKL | HLA-C01:02 | AQP12B; | Pancreas | 1067 |
| | | AQP12A | | |
| MAQSCSSAL | HLA-C03:04 | AQP12B; | Pancreas | 1068 |
| | | AQP12A | | |
| FAREAMRTL | HLA-C03:04 | AQP12A | Pancreas | 1069 |
| FACSGHTLL | HLA-C03:04 | AQP12B; | Pancreas | 1070 |
| | | AQP12A | | |
| FACSGHTL | HLA-C03:04 | AQP12B; | Pancreas | 1071 |
| | | AQP12A | | |
| MAQSCSSAL | HLA-C04:01 | AQP12B; | Pancreas | 1072 |
| | | AQP12A | | |
| AYSGPAVAL | HLA-C04:01 | AQP12B: | Pancreas | 1073 |
| | | AQP12A | | |
| MRLCWAWEL | HLA-C04:01 | AQP12A | Pancreas | 1074 |
| FFNPALAASV | HLA-C04:01 | AQP12B; | Pancreas | 1075 |
| | | AQP12A | | |
| AFCFHLTLL | HLA-C04:01 | AQP12B; | Pancreas | 1076 |
| | | AQP12A | | |
| CFHLTLLHL | HLA-C04:01 | AQP12B | Pancreas | 1077 |
| | | AQP12A | | |
| MRLCWAWEL | HLA-C07:01 | AQP12A | Pancreas | 1078 |
| FAREAMRTL | HLA-C07:01 | AQP12A | Pancreas | 1079 |
| YRAPRGKPA | HLA-C07:01 | AQP12B: | Pancreas | 1080 |
| | | AQP12A | | |
| TLLEYVQVY | HLA-C07:01 | AQP12B; | Pancreas | 1081 |
| | | AQP12A | | |
| CSGHTLLEY | HLA-C07:01 | AQP12B; | Pancreas | 1082 |
| | | AQP12A | | |
| MRLCWAWEL | HLA-C07:02 | AQP12A | Pancreas | 1083 |
| FAREAMRTL | HLA-C07:02 | AQP12A | Pancreas | 1084 |
| SFFFATFAL | HLA-C07:02 | AQP12A | Pancreas | 1085 |
| AYSGPAVAL | HLA-C07:02 | AQP12B; | Pancreas | 1086 |
| | | AQP12A | | |
| FGPDLLLTL | HLA-C07:02 | AQP12B; | Pancreas | 1087 |
| | | AQP12A | | |
| FLEMRTLVEL | HLA-A01:01 | AQP12B | Pancreas | 1088 |
| LTRLCWAWEL | HLA-A01:01 | AQP12B | Pancreas | 1089 |
| FATFTLCEA | HLA-A01:01 | AQP12B | Pancreas | 1090 |
| CTLTRLCWA | HLA-A01:01 | AQP12B | Pancreas | 1091 |
| FLEMRTLV | HLA-A01:01 | AQP12B | Pancreas | 1092 |
| ESLPGTLLK | HLA-A01:01 | AQP12B | Pancreas | 1093 |
| FLEMRTLVE | HLA-A01:01 | AQP12B | Pancreas | 1094 |
| FLEMRTLVEL | HLA-A02:01 | AQP12B | Pancreas | 1095 |
| FLMAEESLPGT | HLA-A02:01 | AQP12B | Pancreas | 1096 |
| CFLEMRTLVEL | HLA-A02:01 | AQP12B | Pancreas | 1097 |
| RLHPDAPLL | HLA-A02:01 | AQP12B | Pancreas | 1098 |
| ACFLEMRTLVEL | HLA-A02:01 | AQP12B | Pancreas | 1099 |
| FLEMRTLVELG | HLA-A02:01 | AQP12B | Pancreas | 1100 |
| TLTRICWAWEL | HLA-A02:01 | AQP12B | Pancreas | 1101 |
| FLMAEESL | HLA-A02:01 | AQP12B | Pancreas | 1102 |
| EFLMAEESLPGT | HLA-A02:01 | AQP12B | Pancreas | 1103 |
| RLHPDAPLLGL | HLA-A02:01 | AQP12B | Pancreas | 1104 |
| TLCEAARRA | HLA-A02:01 | AQP12B | Pancreas | 1105 |
| FLMAEESLP | HLA-A02:01 | AQP12B | Pancreas | 1106 |
| FLEMRTLV | HLA-A02:01 | AQP12B | Pancreas | 1107 |
| SLSFFFATFTL | HLA-A02:01 | AQP12B | Pancreas | 1108 |
| GMQAACTLTR | HLA-A03:01 | AQP12B | Pancreas | 1109 |
| MAEESLPGTLLK | HLA-A03:01 | AQP12B | Pancreas | 1110 |
| TLCEAARRASK | HLA-A03:01 | AQP12B | Pancreas | 1111 |
| AEESLPGTLLK | HLA-A03:01 | AQP12B | Pancreas | 1112 |
| ESLPGTLLK | HLA-A03:01 | AQP12B | Pancreas | 1113 |
| RLHPDAPLLGL | HLA-A03:01 | AQP12B | Pancreas | 1114 |
| RLHPDAPLLG | HLA-A03:01 | AQP12B | Pancreas | 1115 |
| ESLPGTLLK | HLA-A11:01 | AQP12B | Pancreas | 1116 |
| MAEESLPGTLLK | HLA-A11:01 | AQP12B | Pancreas | 1117 |
| AEESLPGTLLK | HLA-A11:01 | AQP12B | Pancreas | 1118 |
| ATFTLCEAAR | HLA-A11:01 | AQP12B | Pancreas | 1119 |
| GTGAGHAGR | HLA-A11:01 | AQP12B | Pancreas | 1120 |
| SFFFATFTL | HLA-A24:02 | AQP12B | Pancreas | 1121 |
| LSFFFATFTL | HLA-A24:02 | AQP12B | Pancreas | 1122 |
| SLSFFFATFTL | HLA-A24:02 | AQP12B | Pancreas | 1123 |
| RLHPDAPLL | HLA-A24:02 | AQP12B | Pancreas | 1124 |
| FFFATTL | HLA-A24:02 | AQP12B | Pancreas | 1125 |
| RGVSAWHAA | HLA-A30:01 | AQP12B | Pancreas | 1126 |
| HGRGVSAWHA | HLA-A30:01 | AQP12B | Pancreas | 1127 |
| CTLTRLCWA | HLA-A30:01 | AQP12B | Pancreas | 1128 |
| RLHPDAPLL | HLA-A30:01 | AQP12B | Pancreas | 1129 |
| ESLPGTLLK | HLA-A30:01 | AQP12B | Pancreas | 1130 |
| ACFLEMRTL | HLA-A30:01 | AQP12B | Pancreas | 1131 |
| TFTLCEAAR | HLA-A33:03 | AQP12B | Pancreas | 1132 |
| ATFTLCEAAR | HLA-A33:03 | AQP12B | Pancreas | 1133 |
| FTLCEAARR | HLA-A33:03 | AQP12B | Pancreas | 1134 |
| ESLPGTLLK | HLA-A33:03 | AQP12B | Pancreas | 1135 |
| VPHGRGVSA | HLA-B07:02 | AQP12B | Pancreas | 1136 |
| HPDAPLLGL | HLA-B07:02 | AQP12B | Pancreas | 1137 |
| GVPHGRGVSA | HLA-B07:02 | AQP12B | Pancreas | 1138 |
| VPHGRGVSAW | HLA-B07:02 | AQP12B | Pancreas | 1139 |
| FLEMRTLVEL | HLA-B08:01 | AQP12B | Pancreas | 1140 |
| VPHGRGVSA | HLA-B08:01 | AQP12B | Pancreas | 1141 |
| FLEMRTLV | HLA-B08:01 | AQP12B | Pancreas | 1142 |
| ESLPGTLL | HLA-B08:01 | AQP12B | Pancreas | 1143 |
| LSFFFATFTL | HLA-B13:02 | AQP12B | Pancreas | 1144 |
| FLEMRTLVEL | HLA-B13:02 | AQP12B | Pancreas | 1145 |
| MQAACTLTRL | HLA-B13:02 | AQP12B | Pancreas | 1146 |
| RLHPDAPLL | HLA-B13:02 | AQP12B | Pancreas | 1147 |
| AVGAVQLGA | HLA-B13:02 | AQP12B | Pancreas | 1148 |
| HPDAPLLGL | HLA-B13:02 | AQP12B | Pancreas | 1149 |
| GAVQLGACF | HLA-B46:01 | AQP12B | Pancreas | 1150 |
| FATFTLCEA | HLA-B46:01 | AQP12B | Pancreas | 1151 |
| MQAACTLTRL | HLA-B46:01 | AQP12B | Pancreas | 1152 |
| VSAWHAAEA | HLA-B46:01 | AQP12B | Pancreas | 1153 |
| VPHGRGVSA | HLA-B46:01 | AQP12B | Pancreas | 1154 |
| HAAEAGGTG | HLA-B46:01 | AQP12B | Pancreas | 1155 |
| RLHPDAPLL | HLA-C01:02 | AQP12B | Pancreas | 1156 |
| SFFFATFTL | HLA-C01:02 | AQP12B | Pancreas | 1157 |
| FLEMRTLVEL | HLA-C01:02 | AQP12B | Pancreas | 1158 |
| LHPDAPLLGL | HLA-C01:02 | AQP12B | Pancreas | 1159 |
| RLHPDAPL | HLA-C01:02 | AQP12B | Pancreas | 1160 |
| FATFTLCEA | HLA-C03:04 | AQP12B | Pancreas | 1161 |
| QAACTLTRL | HLA-C03:04 | AQP12B | Pancreas | 1162 |
| MQAACTLTRL | HLA-C03:04 | AQP12B | Pancreas | 1163 |
| FFFATFTL | HLA-C03:04 | AQP12B | Pancreas | 1164 |
| ESLPGTLL | HLA-C03:04 | AQP12B | Pancreas | 1165 |
| FLMAEESL | HLA-C03:04 | AQP12B | Pancreas | 1166 |
| RLHPDAPLL | HLA-C04:01 | AQP12B | Pancreas | 1167 |
| TRLCWAWEL | HLA-C04:01 | AQP12B | Pancreas | 1168 |
| LHPDAPLLGL | HLA-C04:01 | AQP12B | Pancreas | 1169 |
| FFFATFTL | HLA-C04:01 | AQP12B | Pancreas | 1170 |
| SFFFATFTL | HLA-C04:01 | AQP12B | Pancreas | 1171 |
| FFFATFTLC | HLA-C04:01 | AQP12B | Pancreas | 1172 |
| TRLCWAWEL | HLA-C07:01 | AQP12B | Pancreas | 1173 |
| SFFFATFTL | HLA-C07:01 | AQP12B | Pancreas | 1174 |
| LTRLCWAWEL | HLA-C07:01 | AQP12B | Pancreas | 1175 |
| ACFLEMRTL | HLA-C07:01 | AQP12B | Pancreas | 1176 |
| GRLHPDAPLL | HLA-C07:01 | AQP12B | Pancreas | 1177 |
| TRICWAWEL | HLA-C07:02 | AQP12B | Pancreas | 1178 |
| SFFFATFTL | HLA-C07:02 | AQP12B | Pancreas | 1179 |
| RLHPDAPLL | HLA-C07:02 | AQP12B | Pancreas | 1180 |
| LHPDAPLL | HLA-C07:02 | AQP12B | Pancreas | 1181 |
| FFFATFTL | HLA-C07:02 | AQP12B | Pancreas | 1182 |
| NSDFAPHPY | HLA-A01:01 | C2orf53 | Testis | 1183 |
| SNSDFAPHPY | HLA-A01:01 | C2orf53 | Testis | 1184 |
| CDSNSDFAPHPY | HLA-A01:01 | C2orf53 | Testis | 1185 |
| ASSPSNHWLY | HLA-A01:01 | C2orf53 | Testis | 1186 |
| SSPSNHWLY | HLA-A01:01 | C2orf53 | Testis | 1187 |
| RLLAFPQLL | HLA-A02:01 | C2orf53 | Testis | 1188 |
| RLLAFPQLLPCV | HLA-A02:01 | C2orf53 | Testis | 1189 |
| YMPGVPPA | HLA-A02:01 | C2orf53 | Testis | 1190 |
| ALASVQGHWV | HLA-A02:01 | C2orf53 | Testis | 1191 |
| ALAQALVVQL | HLA-A02:01 | C2orf53 | Testis | 1192 |
| WLYPSPPLT | HLA-A02:01 | C2orf53 | Testis | 1193 |
| LLAFPQLLPCV | HLA-A02:01 | C2orf53 | Testis | 1194 |
| WLYPSPPL | HLA-A02:01 | C2orf53 | Testis | 1195 |
| PRLLAFPQLL | HLA-A02:01 | C2orf53 | Testis | 1196 |
| PLLPPPQV | HLA-A02:01 | C2orf53 | Testis | 1197 |
| SVQGHWVRV | HLA-A02:01 | C2orf53 | Testis | 1198 |
| QLVDSSPHNL | HLA-A02:01 | C2orf53 | Testis | 1199 |
| RLLAFPQL | HLA-A02:01 | C2orf53 | Testis | 1200 |
| YLSLPRPRA | HLA-A02:01 | C2orf53 | Testis | 1201 |
| RQTWRWHQYR | HLA-A03:01 | C2orf53 | Testis | 1202 |
| RALHLLPEK | HLA-A03:01 | C2orf53 | Testis | 1203 |
| RLHSNRQTWR | HLA-A03:01 | C2orf53 | Testis | 1204 |
| RLLAFPQLLPC | HLA-A03:01 | C2orf53 | Testis | 1205 |
| VSLELILQK | HLA-A03:01 | C2orf53 | Testis | 1206 |
| WLYPSPPLTP | HLA-A03:01 | C2orf53 | Testis | 1207 |
| VSLELILQK | HLA-A11:01 | C2orf53 | Testis | 1208 |
| ATAFAAPVAR | HLA-A11:01 | C2orf53 | Testis | 1209 |
| RALHLLPEK | HLA-A11:01 | C2orf53 | Testis | 1210 |
| QTWRWHQYR | HLA-A11:01 | C2orf53 | Testis | 1211 |
| ASVQGHWVR | HLA-A11:01 | C2orf53 | Testis | 1212 |
| LYPSPPLTPSF | HLA-A24:02 | C2orf53 | Testis | 1213 |
| FFHQNYLSL | HLA-A24:02 | C2orf53 | Testis | 1214 |
| RYRTGPRLLAF | HLA-A24:02 | C2orf53 | Testis | 1215 |
| RLLLLQHLW | HLA-A24:02 | C2orf53 | Testis | 1216 |
| SLPSSPTFF | HLA-A24:02 | C2orf53 | Testis | 1217 |
| RYRTGPRLL | HLA-A24:02 | C2orf53 | Testis | 1218 |
| RTRPAATAFA | HLA-A30:01 | C2orf53 | Testis | 1219 |
| RTRPAATAFAA | HLA-A30:01 | C2orf53 | Testis | 1220 |
| HRTRPAATAFA | HLA-A30:01 | C2orf53 | Testis | 1221 |
| VSLELILQK | HLA-A30:01 | C2orf53 | Testis | 1222 |
| RYRTGPRLL | HLA-A30:01 | C2orf53 | Testis | 1223 |
| SVLPGRPTW | HLA-A30:01 | C2orf53 | Testis | 1224 |
| QTWRWHQYR | HLA-A33:03 | C2orf53 | Testis | 1225 |
| NRQTWRWHQYR | HLA-A33:03 | C2orf53 | Testis | 1226 |
| RQTWRWHQYR | HLA-A33:03 | C2orf53 | Testis | 1227 |
| TAFAAPVAR | HLA-A33:03 | C2orf53 | Testis | 1228 |
| NYLSLPRPR | HLA-A33:03 | C2orf53 | Testis | 1229 |
| RPRSPSCPL | HLA-B07:02 | C2orf53 | Testis | 1230 |
| APKQVTTSL | HLA-B07:02 | C2orf53 | Testis | 1231 |
| RPTWGFSQL | HLA-B07:02 | C2orf53 | Testis | 1232 |
| LPRYRTGPRL | HLA-B07:02 | C2orf53 | Testis | 1233 |
| LRPRSPSCPL | HLA-B07:02 | C2orf53 | Testis | 1234 |
| SPSLPSSPTE | HLA-B07:02 | C2orf53 | Testis | 1235 |
| SPSQPQNSSL | HLA-B07:02 | C2orf53 | Testis | 1236 |
| LPRYRTGPRL | HLA-B08:01 | C2orf53 | Testis | 1237 |
| CLRPRSPSCPL | HLA-B08:01 | C2orf53 | Testis | 1238 |
| LPRYRTGPRLL | HLA-B08:01 | C2orf53 | Testis | 1239 |
| PGPKRPVSL | HLA-B08:01 | C2orf53 | Testis | 1240 |
| APKQVTTSL | HLA-B08:01 | C2orf53 | Testis | 1241 |
| GPKRPVSL | HLA-B08:01 | C2orf53 | Testis | 1242 |
| VEYPICLVCL | HLA-B13:02 | C2orf53 | Testis | 1243 |
| RPTWGFSQLV | HLA-B13:02 | C2orf53 | Testis | 1244 |
| GQAPVVEYPI | HLA-B13:02 | C2orf53 | Testis | 1245 |
| GQESGPLRI | HLA-B13:02 | C2orf53 | Testis | 1246 |
| GQARALALL | HLA-B13:02 | C2orf53 | Testis | 1247 |
| RLLAFPQLL | HLA-B13:02 | C2orf53 | Testis | 1248 |
| RTRPAATAF | HLA-B46:01 | C2orf53 | Testis | 1249 |
| HRTRPAATAP | HLA-B46:01 | C2orf53 | Testis | 1250 |
| RTGPRLLAF | HLA-B46:01 | C2orf53 | Testis | 1251 |
| FFHQNYLSL | HLA-B46:01 | C2orf53 | Testis | 1252 |
| FSGPPPRAP | HLA-B46:01 | C2orf53 | Testis | 1253 |
| PSPPLTPSF | HLA-B46:01 | C2orf53 | Testis | 1254 |
| FAPHPYSPSL | HLA-C01:02 | C2orf53 | Testis | 1255 |
| SSPHNLQPL | HLA-C01:02 | C2orf53 | Testis | 1256 |
| SLPSSPTFF | HLA-C01:02 | C2orf53 | Testis | 1257 |
| FFHQNYLSL | HLA-C01:02 | C2orf53 | Testis | 1258 |
| RLPQGQARAL | HLA-C01:02 | C2orf53 | Testis | 1259 |
| AATAFAAPV | HLA-C03:04 | C2orf53 | Testis | 1260 |
| FAPHPYSPSL | HLA-C03:04 | C2orf53 | Testis | 1261 |
| IAHDLRLLL | HLA-C03:04 | C2orf53 | Testis | 1262 |
| FAAPVARQN | HLA-C03:04 | C2orf53 | Testis | 1263 |
| FFHQNYLSL | HLA-C04:01 | C2orf53 | Testis | 1264 |
| SLPSSPTFF | HLA-C04:01 | C2orf53 | Testis | 1265 |
| AHDLRLLLL | HLA-C04:01 | C2orf53 | Testis | 1266 |
| LVDSSPHNL | HLA-C04:01 | C2orf53 | Testis | 1267 |
| YRTGPRLLAF | HLA-C07:01 | C2orf53 | Testis | 1268 |
| RRIAHDLRL | HLA-C07:01 | C2orf53 | Testis | 1269 |
| YRTGPRLLA | HLA-C07:01 | C2orf53 | Testis | 1270 |
| RRIAHDLRLL | HLA-C07:01 | C2orf3 | Testis | 1271 |
| KRPVSLEL | HLA-C07:01 | C2orf53 | Testis | 1272 |
| TGQAPVVEY | HLA-C07:01 | C2orf53 | Testis | 1273 |
| FFHQNYLSL | HLA-C07:02 | C2orf53 | Testis | 1274 |
| YRTGPRLLAF | HLA-C07:02 | C2orf53 | Testis | 1275 |
| RRIAHDLRL | HLA-C07:02 | C2orf53 | Testis | 1276 |
| KRPVSLEL | HLA-C07:02 | C2orf53 | Testis | 1277 |
| YRTGPRLL | HLA-C07:02 | C2orf53 | Testis | 1278 |
| NRPGPSRGVSY | HLA-C07:02 | C2orf53 | Testis | 1279 |
| WTFRGKIHAR | HLA-A01:01 | CCDC70 | Testis | 1280 |
| WMENNGHIA | HLA-A01:01 | CCDC70 | Testis | 1281 |
| WVEERALLE | HLA-A01:01 | CCDC70 | Testis | 1282 |
| LLEGEKALW | HLA-A01:01 | CCDC70 | Testis | 1283 |
| LWEDKTSLW | HLA-A01:01 | CCDC70 | Testis | 1284 |
| LWEEENALW | HLA-A01:01 | CCDC70 | Testis | 1285 |
| SLWEEENAL | HLA-A02:01 | CCDC70 | Testis | 1286 |
| ALWEDKTSL | HLA-A02:01 | CCDC70 | Testis | 1287 |
| SLAASSPSI | HLA-A02:01 | CCDC70 | Testis | 1288 |
| ALWEEEKAL | HLA-A02:01 | CCDC70 | Testis | 1289 |
| ALWERDRNL | HLA-A02:01 | CCDC70 | Testis | 1290 |
| ALWERDRNLL | HLA-A02:01 | CCDC70 | Testis | 1291 |
| ALWEEEKALWV | HLA-A02:01 | CCDC70 | Testis | 1292 |
| ALLEGEKAL | HLA-A02:01 | CCDC70 | Testis | 1293 |
| ALWVEERALL | HLA-A02:01 | CCDC70 | Testis | 1294 |
| MLEDGPHNA | HLA-A02:01 | CCDC70 | Testis | 1295 |
| RLIRKMFSFK | HLA-A03:01 | CCDC70 | Testis | 1296 |
| LIRKMFSFK | HLA-A03:01 | CCDC70 | Testis | 1297 |
| KMFSFKVSR | HLA-A03:01 | CCDC70 | Testis | 1298 |
| ATPPFRLIRK | HLA-A03:01 | CCDC70 | Testis | 1299 |
| ASSPSIRQK | HLA-A03:01 | CCDC70 | Testis | 1300 |
| ATPPFRLIRK | HLA-A11:01 | CCDC70 | Testis | 1301 |
| RLIRKMFSFK | HLA-A11:01 | CCDC70 | Testis | 1302 |
| KTFWKKYRTFWK | HLA-A11:01 | CCDC70 | Testis | 1303 |
| ASSPSIRQK | HLA-A11:01 | CCDC70 | Testis | 1304 |
| ATPPFRLIR | HLA-A11:01 | CCDC70 | Testis | 1305 |
| RLIRKMFSF | HLA-A24:02 | CCDC70 | Testis | 1306 |
| TFWKKYRTF | HLA-A24:02 | CCDC70 | Testis | 1307 |
| RWMGLACF | HLA-A24:02 | CCDC70 | Testis | 1308 |
| PFWEEEKTP | HLA-A24:02 | CCDC70 | Testis | 1309 |
| IFREKIEDF | HLA-A24:02 | CCDC70 | Testis | 1310 |
| RLIRKMPSFK | HLA-A30:01 | CCDC70 | Testis | 1311 |
| LIRKMFSFK | HLA-A30:01 | CCDC70 | Testis | 1312 |
| KYRTFWKEDK | HLA-A30:01 | CCDC70 | Testis | 1313 |
| KYRTFWKED | HLA-A30:01 | CCDC70 | Testis | 1314 |
| ASSPSIRQK | HLA-A30:01 | CCDC70 | Testis | 1315 |
| SSPSIRQKK | HLA-A30:01 | CCDC70 | Testis | 1316 |
| TFRGKIHAFR | HLA-A33:03 | CCDC70 | Testis | 1317 |
| SFWEMEKSFR | HLA-A33:03 | CCDC70 | Testis | 1318 |
| DFREEMWTFR | HLA-A33:03 | CCDC70 | Testis | 1319 |
| EDGPHNANR | HLA-A33:03 | CCDC70 | Testis | 1320 |
| ENALWEEER | HLA-A33:03 | CCDC70 | Testis | 1321 |
| SPSIRQKKL | HLA-B07:02 | CCDC70 | Testis | 1322 |
| SPSIRQKKLM | HLA-B07:02 | CCDC70 | Testis | 1323 |
| SSPSIRQKKL | HLA-B07:02 | CCDC70 | Testis | 1324 |
| IHAFRGQIL | HLA-B07:02 | CCDC70 | Testis | 1325 |
| FRLIRKMFSF | HLA-B08:01 | CCDC70 | Testis | 1326 |
| TFRGKIHAF | HLA-B08:01 | CCDC70 | Testis | 1327 |
| RLIRKMFSF | HLA-B08:01 | CCDC70 | Testis | 1328 |
| ALWEDKTSL | HLA-B08:01 | CCDC70 | Testis | 1329 |
| FWKEDNAL | HLA-B08:01 | CCDC70 | Testis | 1330 |
| ALWERDRNL | HLA-B08:01 | CCDC70 | Testis | 1331 |
| RSLAASSPSI | HLA-B13:02 | CCDC70 | Testis | 1332 |
| MATPPFRLI | HLA-B13:02 | CCDC70 | Testis | 1333 |
| REEMWTFRGKI | HLA-B13:02 | CCDC70 | Testis | 1334 |
| RQKKLMHKL | HLA-B13:02 | CCDC70 | Testis | 1335 |
| KAFREEMKI | HLA-B13:02 | CCDC70 | Testis | 1336 |
| GQILGFWEE | HLA-B13:02 | CCDC70 | Testis | 1337 |
| FSFKVSRWM | HLA-B46:01 | CCDC70 | Testis | 1338 |
| WTFRGKIHAP | HLA-B46:01 | CCDC70 | Testis | 1339 |
| HAFRGQILGF | HLA-B46:01 | CCDC70 | Testis | 1340 |
| VSRWMGLAC | HLA-B46:01 | CCDC70 | Testis | 1341 |
| TFRGKIHAF | HLA-B46:01 | CCDC70 | Testis | 1342 |
| ALWEDKTSL | HLA-B46:01 | CCDC70 | Testis | 1343 |
| MATPPFRLI | HLA-C0L:02 | CCDC70 | Testis | 1344 |
| SLAASSPSI | HLA-C01:02 | CCDC70 | Testis | 1345 |
| FSFKVSRWM | HLA-C01:02 | CCDC70 | Testis | 1346 |
| ALWEDKTSL | HLA-C01:02 | CCDC70 | Testis | 1347 |
| SLWEEENAL | HLA-C01:02 | CCDC70 | Testis | 1348 |
| ALWEEEKAL | HLA-C01:02 | CCDC70 | Testis | 1349 |
| MATPPERLI | HLA-C03:04 | CCDC70 | Testis | 1350 |
| FSFKVSRWM | HLA-C03:04 | CCDC70 | Testis | 1351 |
| MGLACFRSI | HLA-C03:04 | CCDC70 | Testis | 1352 |
| HAFRGQIL | HLA-C03:04 | CCDC70 | Testis | 1353 |
| MATPPFRLI | HLA-C04:01 | CCDC70 | Testis | 1354 |
| FWEEERPFW | HLA-C04:01 | CCDC70 | Testis | 1355 |
| FWMENNGHI | HLA-C04:01 | CCDC70 | Testis | 1356 |
| SFWEMEKSP | HLA-C04:01 | CCDC70 | Testis | 1357 |
| FWEEEKTF | HLA-C04:01 | CCDC70 | Testis | 1358 |
| LWEEENAL | HLA-C04:01 | CCDC70 | Testis | 1359 |
| NRGQRLLAF | HLA-C07:01 | CCDC70 | Testis | 1360 |
| FSFKVSRWM | HLA-C07:01 | CCDC70 | Testis | 1361 |
| FKVSRWMGL | HLA-C07:01 | CCDC70 | Testis | 1362 |
| FRGQILGF | HLA-C07:01 | CCDC70 | Testis | 1363 |
| FRGKIHAF | HLA-C07:01 | CCDC70 | Testis | 1364 |
| ANRGQRLL | HLA-C07:01 | CCDC70 | Testis | 1365 |
| NRGQRLLAF | HLA-C07:02 | CCDC70 | Testis | 1366 |
| TFRGKIHAF | HLA-C07:02 | CCDC70 | Testis | 1367 |
| TFWKKYRTF | HLA-C07:02 | CCDC70 | Testis | 1368 |
| FRLIRKMFSF | HLA-C07:02 | CCDC70 | Testis | 1369 |
| FRGKIHAF | HLA-C07:02 | CCDC70 | Testis | 1370 |
| FRGQILGF | HLA-C07:02 | CCDC70 | Testis | 1371 |
| FREEMWTF | HLA-C07:02 | CCDC70 | Testis | 1372 |
| LSCGDPTYPPY | HLA-A01:01 | CELA2A | Pancreas | 1373 |
| CGDPTYPPY | HLA-A01:01 | CELAZA | Pancreas | 1374 |
| LTDKIQLACL | HLA-A01:01 | CELA2A; | Pancreas | 1375 |
| | | CELA2B | | |
| ALSCGDPTYPPY | HLA-A01:01 | CELA2A | Pancreas | 1376 |
| LTDKIQLAC | HLA-A01:01 | CELA2A; | Pancreas | 1377 |
| | | CELA2B | | |
| NSWPWQVSLQY | HLA-A01:01 | CELA2A; | Pancreas | 1378 |
| | | CELA2B | | |
| LTDKIQLA | HLA-A01:01 | CELA2A; | Pancreas | 1379 |
| | | CELA2B | | |
| SLIANSWVL | HLA-A02:01 | CELA2A; | Pancreas | 1380 |
| | | CELA2B | | |
| ILPNNYPCYV | HLA-A02:01 | CELA2A; | Pancreas | 1381 |
| | | CELA2B | | |
| ALLKLANPV | HLA-A02:01 | CELAZA: | Pancreas | 1382 |
| | | CELA2B | | |
| GLGRHNLYV | HLA-A02:01 | CELA2A | Pancreas | 1383 |
| LLSTLVAGA | HLA-A02:01 | CELA2A; | Pancreas | 1384 |
| | | CELA2B | | |
| LLLSTLVAGA | HLA-A02:01 | CELA2A; | Pancreas | 1385 |
| | | CELA2B | | |
| YIDWINSVI | HLA-A02:01 | CELA2A | Pancreas | 1386 |
| IQLACLPPA | HLA-A02:01 | CELA2A; | Pancreas | 1387 |
| | | CELA2B | | |
| SLIANSWV | HLA-A02:01 | CELA2A; | Pancreas | 1388 |
| | | CELA2B | | |
| LLSTLVAGAL | HLA-A02:01 | CELA2A; | Pancreas | 1389 |
| | | CELA2B | | |
| SLTDKIQLA | HLA-A02:01 | CELA2A; | Pancreas | 1390 |
| | | CELA2B | | |
| VLQQGRLLV | HLA-A02:01 | CELA2A | Pancreas | 1391 |
| KLANPVSL | HLA-A02:01 | CELA2A; | Pancreas | 1392 |
| | | CELA2B | | |
| SLTDKIQL | HLA-A02:01 | CELA2A; | Pancreas | 1393 |
| | | CELA2B | | |
| YVAESGSLAV | HLA-A02:01 | CELA2A; | Pancreas | 1394 |
| | | CELA2B | | |
| VLQQGRLLVV | HLA-A02:01 | CELA2A | Pancreas | 1395 |
| KLANPVSLTDK | HLA-A03:01 | CELA2A; | Pancreas | 1396 |
| | | CELA2B | | |
| SSAWWGSSVK | HLA-A03:01 | CELA2A | Pancreas | 1397 |
| RTYRVGLGR | HLA-A03:01 | CELA2A | Pancreas | 1398 |
| LKLANPVSLTDK | HLA-A03:01 | CELA2A; | Pancreas | 1399 |
| | | CELA2B | | |
| SVFTRVSNY | HLA-A03:01 | CELA2A | Pancreas | 1400 |
| SVSKIVVHK | HLA-A11:01 | CELAZA; | Pancreas | 1401 |
| | | CELA2B | | |
| GSLAVSVSK | HLA-A11:01 | CELA2A; | Pancreas | 1402 |
| | | CELA2B | | |
| SGSLAVSVSK | HLA-A11:01 | CELA2A; | Pancreas | 1403 |
| | | CELA2B | | |
| PTYPPYVTR | HLA-A11:01 | CELA2A | Pancreas | 1404 |
| RWQVHGIVSF | HLA-A24:02 | CELA2A | Pancreas | 1405 |
| TYPPYVTRV | HLA-A24:02 | CELA2A | Pancreas | 1406 |
| NYYHKPSVF | HLA-A24:02 | CELA2A | Pancreas | 1407 |
| NYIDWINSVI | HLA-A24:02 | CELA2A | Pancreas | 1408 |
| NYIDWINSV | HLA-A24:02 | CELA2A | Pancreas | 1409 |
| SSRTYRVGL | HLA-A30:01 | CELA2A | Pancreas | 1410 |
| GSLAVSVSK | HLA-A30:01 | CELA2A; | Pancreas | 1411 |
| | | CELA2B | | |
| SVKTSMICA | HLA-A30:01 | CELA2A | Pancreas | 1412 |
| RTLLLSTLV | HLA-A30:01 | CELA2A; | Pancreas | 1413 |
| | | CELA2B | | |
| KGNDIALLK | HLA-A30:01 | CELA2A; | Pancreas | 1414 |
| | | CELA2B | | |
| SVSKIVVHK | HLA-A30:01 | CELA2A; | Pancreas | 1415 |
| | | CELA2B | | |
| SVFTRVSNY | HLA-A30:01 | CELA2A | Pancreas | 1416 |
| CISSSRTYR | HLA-A33:03 | CELA2A | Pancreas | 1417 |
| HCISSSRTYR | HLA-A33:03 | CELA2A | Pancreas | 1418 |
| YYHKPSVFTR | HLA-A33:03 | CELA2A | Pancreas | 1419 |
| NYYHKPSVFTR | HLA-A33:03 | CELA2A | Pancreas | 1420 |
| PTYPPYVTR | HLA-A33:03 | CELA2A | Pancreas | 1421 |
| RPNSWPWQVSL | HLA-B07:02 | CELA2A; | Pancreas | 1422 |
| | | CELA2B | | |
| YPPYVTRVV | HLA-B07:02 | CELA2A | Pancreas | 1423 |
| ARPNSWPWQVSL | HLA-B07:02 | CELA2A; | Pancreas | 1424 |
| | | CELA2B | | |
| RPNSWPWQV | HLA-B07:02 | CELA2A; | Pancreas | 1425 |
| | | CELA2B | | |
| LPPAGTIL | HLA-B07:02 | CELA2A; | Pancreas | 1426 |
| | | CELA2B | | |
| MIRTLLLSTL | HLA-B08:01 | CELA2A; | Pancreas | 1427 |
| | | CELA2B | | |
| LLKLANPVSL | HLA-B08:01 | CELA2A; | Pancreas | 1428 |
| | | CELA2B | | |
| SSRTYRVGL | HLA-B08:01 | CELA2A | Pancreas | 1429 |
| SLTDKIQL | HLA-B08:01 | CELA2A; | Pancreas | 1430 |
| | | CELA2B | | |
| VLQQGRLL | HLA-B08:01 | CELA2A | Pancreas | 1431 |
| KLANPVSL | HLA-B08:01 | CELA2A; | Pancreas | 1432 |
| | | CELA2B | | |
| RTLLLSTLV | HLA-B13:02 | CELA2A; | Pancreas | 1433 |
| | | CELA2B | | |
| IDWINSVIA | HLA-B13:02 | CELAZA | Pancreas | 1434 |
| SNYIDWINSV | HLA-B13:02 | CELA2A | Pancreas | 1435 |
| RVSNYIDWI | HLA-B13:02 | CELA2A | Pancreas | 1436 |
| SLAVSVSKI | HLA-B13:02 | CELA2A; | Pancreas | 1437 |
| | | CELA2B | | |
| NQISKGNDI | HLA-B13:02 | CELA2A | Pancreas | 1438 |
| YATCSSSAW | HLA-B46:01 | CELA2A | Pancreas | 1439 |
| WQVHGIVSF | HLA-B46:01 | CELA2A | Pancreas | 1440 |
| SVFTRVSNY | HLA-B46:01 | CELA2A | Pancreas | 1441 |
| SSAWWGSSV | HLA-B46:01 | CELA2A | Pancreas | 1442 |
| NSWPWQVSL | HLA-B46:01 | CELA2A; | Pancreas | 1443 |
| | | CELA2B | | |
| CLPPAGTIL | HLA-C01:02 | CELA2A; | Pancreas | 1444 |
| | | CELA2B | | |
| YIDWINSVI | HLA-C01:02 | CELA2A | Pancreas | 1445 |
| NSWPWQVSL | HLA-C01:02 | CELA2A; | Pancreas | 1446 |
| | | CELA2B | | |
| SAWWGSSV | HLA-C01:02 | CELA2A | Pancreas | 1447 |
| SSAWWGSSV | HLA-C03:04 | CELA2A | Pancreas | 1448 |
| YATCSSSAW | HLA-C03:04 | CELA2A | Pancreas | 1449 |
| LSTLVAGAL | HLA-C03:04 | CELA2A; | Pancreas | 1450 |
| | | CELA2B | | |
| NSWPWQVSL | HLA-C03:04 | CELA2A; | Pancreas | 1451 |
| | | CELA2B | | |
| LANPVSLTD | HLA-C03:04 | CELA2A; | Pancreas | 1452 |
| | | CELA2B | | |
| YIDWINSVI | HLA-C04:01 | CELA2A | Pancreas | 1453 |
| WYHTCGGSL | HLA-C04:01 | CELA2A; | Pancreas | 1454 |
| | | CELA2B | | |
| CLPPAGTIL | HLA-C04:01 | CELA2A; | Pancreas | 1455 |
| | | CELA2B | | |
| NYYHKPSVF | HLA-C04:01 | CELA2A | Pancreas | 1456 |
| TYPPYVTRV | HLA-C04:01 | CELA2A | Pancreas | 1457 |
| YYHKPSVF | HLA-C04:01 | CELAZA | Pancreas | 1458 |
| NYIDWINSV | HLA-C07:01 | CELA2A | Pancreas | 1459 |
| IRTLLLSTL | HLA-C07:01 | CELA2A; | Pancreas | 1460 |
| | | CELA2B | | |
| YRVGLGRHNL | HLA-C07:01 | CELA2A | Pancreas | 1461 |
| NSWPWQVSL | HLA-C07:01 | CELA2A; | Pancreas | 1462 |
| | | CELA2B | | |
| TNGAVPDVL | HLA-C07:01 | CELA2A | Pancreas | 1463 |
| LANPVSLTD | HLA-C07:01 | CELA2A; | Pancreas | 1464 |
| | | CELA2B | | |
| NYYHKPSVF | HLA-C07:02 | CELA2A | Pancreas | 1465 |
| TYPPYVTRV | HLA-C07:02 | CELA2A | Pancreas | 1466 |
| NYIDWINSV | HLA-C07:02 | CELA2A | Pancreas | 1467 |
| YYHKPSVF | HLA-C07:02 | CELA2A | Pancreas | 1468 |
| ARPNSWPWQV | HLA-C07:02 | CELA2A; | Pancreas | 1469 |
| | | CELA2B | | |
| LTSVIGQNYY | HLA-A01:01 | CELA2B | Pancreas | 1470 |
| LTSVLGCNY | HLA-A01:01 | CELA2B | Pancreas | 1471 |
| LTSVLGCNYYY | HLA-A01:01 | CELA2B | Pancreas | 1472 |
| YSSNGQWY | HLA-A01:01 | CELA2B | Pancreas | 1473 |
| MLGQHNLYV | HLA-A02:01 | CELA2B | Pancreas | 1474 |
| VMLGQHNLYV | HLA-A02:01 | CELA2B | Pancreas | 1475 |
| LVAGALSCGV | HLA-A02:01 | CELA2B | Pancreas | 1476 |
| MLGQHNLYVA | HLA-A02:01 | CELA2B | Pancreas | 1477 |
| VLQRCSLIL | HLA-A02:01 | CELA2B | Pancreas | 1478 |
| YRVMLGQHNLYV | HLA-A02:01 | CELA2B | Pancreas | 1479 |
| ALSCGVSTYA | HLA-A02:01 | CELA2B | Pancreas | 1480 |
| FVRDLCSSQLEL | HLA-A02:01 | CELA2B | Pancreas | 1481 |
| SLILFFVRDL | HLA-A02:01 | CELA2B | Pancreas | 1482 |
| RVMLGQHNLYV | HLA-A02:01 | CELA2B | Pancreas | 1483 |
| ALPDDLKQG | HLA-A02:01 | CELA2B | Pancreas | 1484 |
| DLKQGQLLV | HLA-A02:01 | CELA2B | Pancreas | 1485 |
| DLCSSQLEL | HLA-A02:01 | CELA2B | Pancreas | 1486 |
| SQLELTGV | HLA-A02:01 | CELA2B | Pancreas | 1487 |
| ALPDDLKQGQL | HLA-A02:01 | CELA2B | Pancreas | 1488 |
| AGSSGIYRV | HLA-A02:01 | CELA2B | Pancreas | 1489 |
| SVLGCNYYYK | HLA-A03:01 | CELA2B | Pancreas | 1490 |
| VLGCNYYYK | HLA-A03:01 | CELA2B | Pancreas | 1491 |
| SSGWWGSTVK | HLA-A03:01 | CELA2B | Pancreas | 1492 |
| SIFTRVSNY | HLA-A03:01 | CELA2B | Pancreas | 1493 |
| SVLGCNYYY | HLA-A03:01 | CELA2B | Pancreas | 1494 |
| SVLQQNYYYK | HLA-A11:01 | CELA2B | Pancreas | 1495 |
| STYAPDMSR | HLA-A11:01 | CELA2B | Pancreas | 1496 |
| SSQWWGSTVK | HLA-A11:01 | CELA2B | Pancreas | 1497 |
| SSSGWWGSTVK | HLA-A11:01 | CELA2B | Pancreas | 1498 |
| GVSAGSSGIYR | HLA-A11:01 | CELA2B | Pancreas | 1499 |
| NYYYKPSIF | HLA-A24:02 | CELA2B | Pancreas | 1500 |
| YYKPSIFTRV | HLA-A24:02 | CELA2B | Pancreas | 1501 |
| CNYYYKPSIF | HLA-A24:02 | CELA2B | Pancreas | 1502 |
| TYAPDMSRM | HLA-A24:02 | CELA2B | Pancreas | 1503 |
| YYYKPSIF | HLA-A24:02 | CELA2B | Pancreas | 1504 |
| AAHCIRQIK | HLA-A30:01 | CELA2B | Pancreas | 1505 |
| KVLQRCSLI | HLA-A30:01 | CELA2B | Pancreas | 1506 |
| RVMLGQHNI | HLA-A30:01 | CELA2B | Pancreas | 1507 |
| MSRMLGGEEA | HLA-A30:01 | CELA2B | Pancreas | 1508 |
| SVLGCNYYYK | HLA-A30:01 | CELA2B | Pancreas | 1509 |
| SIFTRVSNY | HLA-A30:01 | CELA2B | Pancreas | 1510 |
| YYKPSIFTR | HLA-A33:03 | CELA2B | Pancreas | 1511 |
| YYYKPSIFTR | HLA-A33:03 | CELA2B | Pancreas | 1512 |
| STYAPDMSR | HLA-A33:03 | CELA2B | Pancreas | 1513 |
| NYYYKPSIFTR | HLA-A33:03 | CELA2B | Pancreas | 1514 |
| RVMLGQHNL | HLA-B07:02 | CELA2B | Pancreas | 1515 |
| KPSIFTRVS | HLA-B07:02 | CELA2B | Pancreas | 1516 |
| FVRDLCSSQL | HLA-B07:02 | CELA2B | Pancreas | 1517 |
| APDMSRML | HLA-B07:02 | CELA2B | Pancreas | 1518 |
| LPDDLKQGQL | HLA-B07:02 | CELA2B | Pancreas | 1519 |
| APDMSRMLG | HLA-B07:02 | CELA2B | Pancreas | 1520 |
| WGRLQTNGAL | HLA-B08:01 | CELA2B | Pancreas | 1521 |
| IIKKAHKVL | HLA-B08:01 | CELA2B | Pancreas | 1522 |
| KVLQRCSL | HLA-B08:01 | CELA2B | Pancreas | 1523 |
| DLKQGQLL | HLA-B08:01 | CELA2B | Pancreas | 1524 |
| QIIKAHKVL | HLA-B08:01 | CELA2B | Pancreas | 1525 |
| GQWYHTCGGSLI | HLA-B13:02 | CELA2B | Pancreas | 1526 |
| CNYYYKPSI | HLA-B13:02 | CELA2B | Pancreas | 1527 |
| SNYNDWINSV | HLA-B13:02 | CELA2B | Pancreas | 1528 |
| RQIIKAHKV | HLA-B13:02 | CELA2B | Pancreas | 1529 |
| RVSNYNDWI | HLA-B13:02 | CELA2B | Pancreas | 1530 |
| AGSSGIYRV | HLA-B13:02 | CELA2B | Pancreas | 1531 |
| VSAGSSGIY | HLA-B46:01 | CELA2B | Pancreas | 1532 |
| SSSGIYRVM | HLA-B46:01 | CELA2B | Pancreas | 1533 |
| ALSCGVSTY | HLA-B46:01 | CELA2B | Pancreas | 1534 |
| AAHCISSSG | HLA-B46:01 | CELA2B | Pancreas | 1535 |
| VSKGNDIAL | HLA-B46:01 | CELA2B | Pancreas | 1536 |
| YAPDMSRML | HLA-C01:02 | CELA2B | Pancreas | 1537 |
| VLQRCSLIL | HLA-C01:02 | CELA2B | Pancreas | 1538 |
| RVMLGQHNL | HLA-C01:02 | CELA2B | Pancreas | 1539 |
| FVRDLCSSQL | HLA-C01:02 | CELA2B | Pancreas | 1540 |
| YAPDMSRM | HLA-C01:02 | CELA2B | Pancreas | 1541 |
| DLCSSQLEL | HLA-C01:02 | CELA2B | Pancreas | 1542 |
| YAPDMSRML | HLA-C03:04 | CELA2B | Pancreas | 1543 |
| SSSGIYRVM | HLA-C03:04 | CELA2B | Pancreas | 1544 |
| HGIGSLTSV | HLA-C03:04 | CELA2B | Pancreas | 1545 |
| VSKGNDIAL | HLA-C03:04 | CELA2B | Pancreas | 1546 |
| QIIKAHKVL | HLA-C03:04 | CELA2B | Pancreas | 1547 |
| YNDWINSVI | HLA-C04:01 | CELA2B | Pancreas | 1548 |
| FFVRDLCSSQL | HLA-C04:01 | CELA2B | Pancreas | 1549 |
| VRDLCSSQL | HLA-C04:01 | CELA2B | Pancreas | 1550 |
| NYYYKPSIF | HLA-C04:01 | CELA2B | Pancreas | 1551 |
| NYNDWINSV | HLA-C04:01 | CELA2B | Pancreas | 1552 |
| YYYKPSIF | HLA-C04:01 | CELA2B | Pancreas | 1553 |
| NYNDWINSV | HLA-C07:01 | CELA2B | Pancreas | 1554 |
| YRVMLGQHNL | HLA-C07:01 | CELA2B | Pancreas | 1555 |
| VRDLCSSQL | HLA-C07:01 | CELA2B | Pancreas | 1556 |
| NYYYKPSIF | HLA-C07:01 | CELA2B | Pancreas | 1557 |
| SSGIYRVML | HLA-C07:01 | CELA2B | Pancreas | 1558 |
| RVMLGQHNI | HLA-C07:01 | CELA2B | Pancreas | 1559 |
| QRCSLILFF | HLA-C07:01 | CELA2B | Pancreas | 1560 |
| NYYYKPSIF | HLA-C07:02 | CELA2B | Pancreas | 1561 |
| TYAPDMSRM | HLA-C07:02 | CELA2B | Pancreas | 1562 |
| NYNDWINSV | HLA-C07:02 | CELA2B | Pancreas | 1563 |
| YYKPSIFTR | HLA-C07:02 | CELA2B | Pancreas | 1564 |
| YYYKPSIF | HLA-C07:02 | CELA2B | Pancreas | 1565 |
| TIDAKELKV | HLA-A01:01 | CETN1 | Testis | 1566 |
| ISFNDFLAVM | HLA-A01:01 | CETN1 | Testis | 1567 |
| LTEDQKQEV | HLA-A01:01 | CETN1 | Testis | 1568 |
| FNDFLAVMTQKM | HLA-A01:01 | CETN1 | Testis | 1569 |
| TIDAKELK | HLA-A01:01 | CETN1 | Testis | 1570 |
| KISFNDFLAV | HLA-A02:01 | CETN1 | Testis | 1571 |
| FLAVMTQKM | HLA-A02:01 | CETN1 | Testis | 1572 |
| ISFNDFLAV | HLA-A02:01 | CETN1 | Testis | 1573 |
| KISFNDFLA | HLA-A02:01 | CETN1 | Testis | 1574 |
| GKISFNDFLAV | HLA-A02:01 | CETN1 | Testis | 1575 |
| DFLAVMTQKM | HLA-A02:01 | CETN1 | Testis | 1576 |
| TGKISFNDFLAV | HLA-A02:01 | CETN1 | Testis | 1577 |
| FLAVMTQKMS | HLA-A02:01 | CETN1 | Testis | 1578 |
| KISFNDFLAVM | HLA-A02:01 | CETN1 | Testis | 1579 |
| RLFDDDETG | HLA-A02:01 | CETN1 | Testis | 1580 |
| TIDAKELKV | HLA-A02:01 | CETN1 | Testis | 1581 |
| ELTEDQKQEV | HLA-A02:01 | CETN1 | Testis | 1582 |
| RLFDDDETGKI | HLA-A02:01 | CETN1 | Testis | 1583 |
| RLFDDDETGK | HLA-A02:01 | CETN1 | Testis | 1584 |
| GTIDAKELKV | HLA-A02:01 | CETN1 | Testis | 1585 |
| NLKRVANEL | HLA-A02:01 | CETN1 | Testis | 1586 |
| AVMTQKMSEK | HLA-A03:01 | CETN1 | Testis | 1587 |
| KPSAASTGQK | HLA-A03:01 | CETN1 | Testis | 1588 |
| AMRALGFEPRK | HLA-A03:01 | CETN1 | Testis | 1589 |
| RLFDDDETGK | HLA-A03:01 | CETN1 | Testis | 1590 |
| RALGFEPRK | HLA-A03:01 | CETN1 | Testis | 1591 |
| AVMTQKMSEK | HLA-A11:01 | CETN1 | Testis | 1592 |
| GTIDAKELK | HLA-A11:01 | CETN1 | Testis | 1593 |
| SGTIDAKELK | HLA-A11:01 | CETN1 | Testis | 1594 |
| RALGFEPRK | HLA-A11:01 | CETN1 | Testis | 1595 |
| EILKAPRLF | HLA-A24:02 | CETN1 | Testis | 1596 |
| SFNDFLAVM | HLA-A24:02 | CETN1 | Testis | 1597 |
| ILKAFRLF | HLA-A24:02 | CETN1 | Testis | 1598 |
| EVREAFDLF | HLA-A24:02 | CETN1 | Testis | 1599 |
| GQKRKVAPK | HLA-A30:01 | CETN1 | Testis | 1600 |
| RALGFEPRK | HLA-A30:01 | CETN1 | Testis | 1601 |
| AMRALGFEPRK | HLA-A30:01 | CETN1 | Testis | 1602 |
| GTIDAKELK | HLA-A30:01 | CETN1 | Testis | 1603 |
| RLFDDDETGK | HLA-A30:01 | CETN1 | Testis | 1604 |
| EVNEEEFLR | HLA-A33:03 | CETN1 | Testis | 1605 |
| AMRALGFEPR | HLA-A33:03 | CETN1 | Testis | 1606 |
| DFLAVMTQK | HLA-A33:03 | CETN1 | Testis | 1607 |
| DAKELKVAMR | HLA-A33:03 | CETN1 | Testis | 1608 |
| SAASTGQKR | HLA-A33:03 | CETN1 | Testis | 1609 |
| EPRKEEMKKM | HLA-B07:02 | CETN1 | Testis | 1610 |
| RVANELGENL | HLA-B07:02 | CETN1 | Testis | 1611 |
| KPSAASTGQ | HLA-B07:02 | CETN1 | Testis | 1612 |
| APKPELTED | HLA-B07:02 | CETN1 | Testis | 1613 |
| RKVAPKPEL | HLA-B07:02 | CETN1 | Testis | 1614 |
| EEFLRIMKKTSL | HLA-B08:01 | CETN1 | Testis | 1615 |
| NLKRVANEL | HLA-B08:01 | CETN1 | Testis | 1616 |
| FKNLKRVANEL | HLA-B08:01 | CETN1 | Testis | 1617 |
| DAKELKVAM | HLA-B08:01 | CETN1 | Testis | 1618 |
| TQQKRKVAP | HLA-B08:01 | CETN1 | Testis | 1619 |
| EILKAFRL | HLA-B08:01 | CETN1 | Testis | 1620 |
| ISFNDFLAV | HLA-B13:02 | CETN1 | Testis | 1621 |
| REAFDLFDV | HLA-B13:02 | CETN1 | Testis | 1622 |
| FDVDGSGTI | HLA-B13:02 | CETN1 | Testis | 1623 |
| TIDAKELKV | HLA-B13:02 | CETN1 | Testis | 1624 |
| ASTGQKRKV | HLA-B13:02 | CETN1 | Testis | 1625 |
| FLAVMTQKM | HLA-B46:01 | CETN1 | Testis | 1626 |
| ISFNDFLAV | HLA-B46:01 | CETN1 | Testis | 1627 |
| ISFNDFLAVM | HLA-B46:01 | CETN1 | Testis | 1628 |
| VANELGEN | HLA-B46:01 | CETN1 | Testis | 1629 |
| VANELGENL | HLA-B46:01 | CETN1 | Testis | 1630 |
| DAKELKVAM | HLA-B46:01 | CETN1 | Testis | 1631 |
| FLAVMTQKM | HLA-C01:02 | CETN1 | Testis | 1632 |
| ISFNDFLAV | HLA-C01:02 | CETN1 | Testis | 1633 |
| SFNDFLAVM | HLA-C01:02 | CETN1 | Testis | 1634 |
| VANELGENL | HLA-C01:02 | CETN1 | Testis | 1635 |
| VAPKPELTED | HLA-C01:02 | CETN1 | Testis | 1636 |
| VAPKPELTE | HLA-C01:02 | CETN1 | Testis | 1637 |
| ISFNDFLAV | HLA-C03:04 | CETN1 | Testis | 1638 |
| FLAVMTQKM | HLA-C03:04 | CETN1 | Testis | 1639 |
| VANELGENL | HLA-C03:04 | CETN1 | Testis | 1640 |
| GTIDAKEL | HLA-C03:04 | CETN1 | Testis | 1641 |
| KVAPKPEL | HLA-C03:04 | CETN1 | Testis | 1642 |
| SFNDFLAVM | HLA-C04:01 | CETN1 | Testis | 1643 |
| LFDVDGSGTI | HLA-C04:01 | CETN1 | Testis | 1644 |
| FLAVMTQKM | HLA-C04:01 | CETN1 | Testis | 1645 |
| AFDLFDVDG | HLA-C04:01 | CETN1 | Testis | 1646 |
| SFNDFLAVM | HLA-C07:01 | CETN1 | Testis | 1647 |
| ISFNDFLAV | HLA-C07:01 | CETN1 | Testis | 1648 |
| FLAVMTQKM | HLA-C07:01 | CETN1 | Testis | 1649 |
| REGTGKISF | HLA-C07:01 | CETN1 | Testis | 1650 |
| APKPELTED | HLA-C07:01 | CETN1 | Testis | 1651 |
| KVAPKPEL | HLA-C07:01 | CETN1 | Testis | 1652 |
| SFNDFLAVM | HLA-C07:02 | CETN1 | Testis | 1653 |
| FLAVMTQKM | HLA-C07:02 | CETN1 | Testis | 1654 |
| ISFNDFLAVM | HLA-C07:02 | CETN1 | Testis | 1655 |
| KRKVAPKPEL | HLA-C07:02 | CETN1 | Testis | 1656 |
| KVAPKPEL | HLA-C07:02 | CETN1 | Testis | 1657 |
| RKVAPKPEL | HLA-C07:02 | CETN1 | Testis | 1658 |
| WTAHAIRDFY | HLA-A01:01 | CLDN6 | Ovarian | 1659 |
| CWTAHAIIRDFY | HLA-A01:01 | CLDN6 | Ovarian | 1660 |
| LVALFGLLVY | HLA-A01:01 | CLDN6 | Ovarian | 1661 |
| PSEYPTKNY | HLA-A01:01 | CLDN6 | Ovarian | 1662 |
| ALFGLLVY | HLA-A01:01 | CLDN6 | Ovarian | 1663 |
| ALFGLLVYL | HLA-A02:01 | CLDN6 | Ovarian | 1664 |
| VLTSGIVFV | HLA-A02:01 | CLDN6 | Ovarian | 1665 |
| TLLGWVNGL | HLA-A02:01 | CLDN6 | Ovarian | 1666 |
| TLIPVCWTA | HLA-A02:01 | CLDN6 | Ovarian | 1667 |
| FVISGVLTL | HLA-A02:01 | CLDN6 | Ovarian | 1668 |
| YMARYSTSA | HLA-A02:01 | CLDN6 | Ovarian | 1669 |
| ALLVALFGL | HLA-A02:01 | CLDN6 | Ovarian | 1670 |
| ALFGLLVYLA | HLA-A02:01 | CLDN6 | Ovarian | 1671 |
| FVISGVLTLIPV | HLA-A02:01 | CLDN6 | Ovarian | 1672 |
| ILGVVLTLL | HLA-A02:01 | CLDN6 | Ovarian | 1673 |
| GLLVYLAGA | HLA-A02:01 | CLDN6 | Ovarian | 1674 |
| LVSCALPMWK | HLA-A03:01 | CLDN6 | Ovarian | 1675 |
| VSCALPMWK | HLA-A03:01 | CLDN6 | Ovarian | 1676 |
| GLLVYLAGAK | HLA-A03:01 | CLDN6 | Ovarian | 1677 |
| ALFGLLVYL | HLA-A03:01 | CLDN6 | Ovarian | 1678 |
| ALFGLLVY | HLA-A03:01 | CLDN6 | Ovarian | 1679 |
| LVALFGLLVY | HLA-A03:01 | CLDN6 | Ovarian | 1680 |
| VSCALPMWK | HLA-A11:01 | CLDN6 | Ovarian | 1681 |
| STSAPAISR | HLA-A11:01 | CLDN6 | Ovarian | 1682 |
| LVSCALPMWK | HLA-A11:01 | CLDN6 | Ovarian | 1683 |
| ALFGLLVYL | HLA-A11:01 | CLDN6 | Ovarian | 1684 |
| MWKALLFLTL | HLA-A24:02 | CLDN6 | Ovarian | 1685 |
| RYSTSAPAI | HLA-A24:02 | CLDN6 | Ovarian | 1686 |
| LYLGWAASGL | HLA-A24:02 | CLDN6 | Ovarian | 1687 |
| VIALLVALF | HLA-A24:02 | CLDN6 | Ovarian | 1688 |
| VFVISGVLTL | HLA-A24:02 | CLDN6 | Ovarian | 1689 |
| KARLVLTSG | HLA-A30:01 | CLDN6 | Ovarian | 1690 |
| KARLVLTSGI | HLA-A30:01 | CLDN6 | Ovarian | 1691 |
| MARYSTSAPA | HLA-A30:01 | CLDN6 | Ovarian | 1692 |
| RGPSEYPTK | HLA-A30:01 | CLDN6 | Ovarian | 1693 |
| ALFGLLVYL | HLA-A30:01 | CLDN6 | Ovarian | 1694 |
| GVLTLIPVC | HLA-A30:01 | CLDN6 | Ovarian | 1695 |
| STSAPAISR | HLA-A33:03 | CLDN6 | Ovarian | 1696 |
| YSTSAPAISR | HLA-A33:03 | CLDN6 | Ovarian | 1697 |
| FYNPLVAEAQKR | HLA-A33:03 | CLDN6 | Ovarian | 1698 |
| TSAPAISR | HLA-A33:03 | CLDN6 | Ovarian | 1699 |
| WTAHAIIR | HLA-A33:03 | CLDN6 | Ovarian | 1700 |
| LPMWKVTAF | HLA-B07:02 | CLDN6 | Ovarian | 1701 |
| ALPMWKVTAF | HLA-B07:02 | CLDN6 | Ovarian | 1702 |
| LPMWKVTAFI | HLA-B07:02 | CLDN6 | Ovarian | 1703 |
| LPMWKALLF | HLA-B07:02 | CLDN6 | Ovarian | 1704 |
| LPMWKALLFL | HLA-B07:02 | CLDN6 | Ovarian | 1705 |
| LPMWKVTAF | HLA-B08:01 | CLDN6 | Ovarian | 1706 |
| LPMWKVTAFI | HLA-B08:01 | CLDN6 | Ovarian | 1707 |
| LPMWKALLFLTL | HLA-B08:01 | CLDN6 | Ovarian | 1708 |
| DSKARLVL | HLA-B08:01 | CLDN6 | Ovarian | 1709 |
| VIALLVAL | HLA-B08:01 | CLDN6 | Ovarian | 1710 |
| RALCVIALLV | HLA-B13:02 | CLDN6 | Ovarian | 1711 |
| LTSGIVFVI | HLA-B13:02 | CLDN6 | Ovarian | 1712 |
| SEYPTKNYV | HLA-B13:02 | CLDN6 | Ovarian | 1713 |
| ALCVIALLV | HLA-B13:02 | CLDN6 | Ovarian | 1714 |
| ALFGLLVYL | HLA-B13:02 | CLDN6 | Ovarian | 1715 |
| VLTSGIVFV | HLA-B13:02 | CLDN6 | Ovarian | 1716 |
| FVISGVLTL | HLA-B46:01 | CLDN6 | Ovarian | 1717 |
| MASAGMQIL | HLA-B46:01 | CLDN6 | Ovarian | 1718 |
| VALFQLLVY | HLA-B46:01 | CLDN6 | Ovarian | 1719 |
| IIRDFYNPL | HLA-B46:01 | CLDN6 | Ovarian | 1720 |
| ISRGPSEY | HLA-B46:01 | CLDN6 | Ovarian | 1721 |
| TAHAIIRDF | HLA-B46:01 | CLDN6 | Ovarian | 1722 |
| FVISGVLTL | HLA-C01:02 | CLDN6 | Ovarian | 1723 |
| YLGWAASGL | HLA-C01:02 | CLDN6 | Ovarian | 1724 |
| ALPMWKALL | HLA-C01:02 | CLDN6 | Ovarian | 1725 |
| ALPMWKAL | HLA-C01:02 | CLDN6 | Ovarian | 1726 |
| ALPMWKVTAF | HLA-C01:02 | CLDN6 | Ovarian | 1727 |
| FVISGVLTL | HLA-C03:04 | CLDN6 | Ovarian | 1728 |
| MASAGMQIL | HLA-C03:04 | CLDN6 | Ovarian | 1729 |
| CALPMWKAL | HLA-C03:04 | CLDN6 | Ovarian | 1730 |
| KALLFLTL | HLA-C03:04 | CLDN6 | Ovarian | 1731 |
| LPMWKALLF | HLA-C04:01 | CLDN6 | Ovarian | 1732 |
| RYSTSAPAI | HLA-C04:01 | CLDN6 | Ovarian | 1733 |
| YLGWAASGL | HLA-C04:01 | CLDN6 | Ovarian | 1734 |
| LFGLLVYL | HLA-C04:01 | CLDN6 | Ovarian | 1735 |
| ILGVVLTLL | HLA-C04:01 | CLDN6 | Ovarian | 1736 |
| VFVISGVL | HLA-C04:01 | CLDN6 | Ovarian | 1737 |
| IRDFYNPLV | HLA-C07:01 | CLDN6 | Ovarian | 1738 |
| FVISGVLTL | HLA-C07:01 | CLDN6 | Ovarian | 1739 |
| ARYSTSAPAI | HLA-C07:01 | CLDN6 | Ovarian | 1740 |
| RDFYNPLVA | HLA-C07:01 | CLDN6 | Ovarian | 1741 |
| TSGIVFVI | HLA-C07:01 | CLDN6 | Ovarian | 1742 |
| SRGPSEYP | HLA-C07:01 | CLDN6 | Ovarian | 1743 |
| RYSTSAPAI | HLA-C07:02 | CLDN6 | Ovarian | 1744 |
| FVISGVLTL | HLA-C07:02 | CLDN6 | Ovarian | 1745 |
| IRDFYNPLV | HLA-C07:02 | CLDN6 | Ovarian | 1746 |
| QKRELGASL | HLA-C07:02 | CLDN6 | Ovarian | 1747 |
| SRGPSEYPT | HLA-C07:02 | CLDN6 | Ovarian | 1748 |
| YTYDEYTKGY | HLA-A01:01 | COL10A1 | Breast | 1749 |
| FTCQIPGIYY | HLA-A01:01 | COL10A1 | Breast | 1750 |
| FTCQIPGIY | HLA-A01:01 | COL10A1 | Breast | 1751 |
| MYTYDEYTKGY | HLA-A01:01 | COL10A1 | Breast | 1752 |
| NAESNGLY | HLA-A01:01 | COL10A1 | Breast | 1753 |
| GSDGKPGY | HLA-A01:01 | COL10A1 | Breast | 1754 |
| LTENDQVWL | HLA-A01:01 | COL10A1 | Breast | 1755 |
| GIYYFSYHV | HLA-A02:01 | COL10A1 | Breast | 1756 |
| MLPQIPFLL | HLA-A02:01 | COL10A1 | Breast | 1757 |
| NLVHGVFYA | HLA-A02:01 | COL10A1 | Breast | 1758 |
| FLLLVSLNL | HLA-A02:01 | COL10A1 | Breast | 1759 |
| LLLVSLNLV | HLA-A02:01 | COL10A1 | Breast | 1760 |
| FLLLVSLNLV | HLA-A02:01 | COL10A1 | Breast | 1761 |
| ILSKAYPAL | HLA-A02:01 | COL10A1 | Breast | 1762 |
| MLPQIPFLLI | HLA-A02:01 | COL10A1 | Breast | 1763 |
| MLPQIPFLLLV | HLA-A02:01 | COL10A1 | Breast | 1764 |
| GLYKNGTPV | HLA-A02:01 | COL10A1 | Breast | 1765 |
| GLDGPKGNPGL | HLA-A02:01 | COL10A1 | Breast | 1766 |
| SLSGTPLVSA | HLA-A02:01 | COL10A1 | Breast | 1767 |
| SLSGTPLV | HLA-A02:01 | COL10A1 | Breast | 1768 |
| GLYSSEYV | HLA-A02:01 | COL10A1 | Breast | 1769 |
| GLPGPPGPSA | HLA-A02:01 | COL10A1 | Breast | 1770 |
| GTHVWVGLYK | HLA-A03:01 | COL10A1 | Breast | 1771 |
| VMYTYDEYTK | HLA-A03:01 | COL10A1 | Breast | 1772 |
| VSAFTVILSK | HLA-A03:01 | COL10A1 | Breast | 1773 |
| SAFTVILSK | HLA-A03:01 | COL10A1 | Breast | 1774 |
| AIGTPIPFDK | HLA-A03:01 | COL10A1 | Breast | 1775 |
| AVMPEGFIK | HLA-A03:01 | COL10A1 | Breast | 1776 |
| GLYKNGTPVMY | HLA-A03:01 | COL10A1 | Breast | 1777 |
| AVMPEGFIK | HLA-A11:01 | COL10A1 | Breast | 1778 |
| SAFTVILSK | HLA-A11:01 | COL10A1 | Breast | 1779 |
| GTHVWVGLYK | HLA-A11:01 | COL10A1 | Breast | 1780 |
| VSAFTVILSK | HLA-A11:0] | COL10A1 | Breast | 1781 |
| CQIPGIYYF | HLA-A24:02 | COL10A1 | Breast | 1782 |
| YYFSYHVHV | HLA-A24:02 | COL10A1 | Breast | 1783 |
| MLPQIPFLL | HLA-A24:02 | COL10A1 | Breast | 1784 |
| EYVHSSFSGF | HLA-A24:02 | COL10A1 | Breast | 1785 |
| HYDPRTGIF | HLA-A24:02 | COL10A1 | Breast | 1786 |
| IGPPGIPGF | HLA-A24:02 | COL10A1 | Breast | 1787 |
| AYPAIGTPIPF | HLA-A24:02 | COL10A1 | Breast | 1788 |
| KTQFFIPYT | HLA-A30:01 | COL10A1 | Breast | 1789 |
| KTQFFIPYTI | HLA-A30:01 | COL10A1 | Breast | 1790 |
| RYQMPTGIK | HLA-A30:01 | COL10A1 | Breast | 1791 |
| SAFTVILSK | HLA-A30:01 | COL10A1 | Breast | 1792 |
| HVKGTHVWV | HLA-A30:01 | COL10A1 | Breast | 1793 |
| LVHGVFYAER | HLA-A33:03 | COL10A1 | Breast | 1794 |
| YYFSYHVHVK | HLA-A33:03 | COL10A1 | Breast | 1795 |
| YNRQQHYDPR | HLA-A33:03 | COL10A1 | Breast | 1796 |
| TIKSKGIAVR | HLA-A33:03 | COL10A1 | Breast | 1797 |
| IPFDKILYNR | HLA-A33:03 | COL10A1 | Breast | 1798 |
| PFDKILYNR | HLA-A33:03 | COL10A1 | Breast | 1799 |
| YTIKSKGIAVR | HLA-A33:03 | COL10A1 | Breast | 1800 |
| RPSLSGTPL | HLA-B07:02 | COL10A1 | Breast | 1801 |
| MPTGIKGPL | HLA-B07:02 | COL10A1 | Breast | 1802 |
| RPSLSGTPLV | HLA-B07:02 | COL10A1 | Breast | 1803 |
| QRPSLSGTPL | HLA-B07:02 | COL10A1 | Breast | 1804 |
| LPQIPFLLL | HLA-B07:02 | COL10A1 | Breast | 1805 |
| IPFLLLVSL | HLA-B07:02 | COL10A1 | Breast | 1806 |
| LPGPPGPSAV | HLA-B07:02 | COL10A1 | Breast | 1807 |
| FIKAGQRPSL | HLA-B08:01 | COL10A1 | Breast | 1808 |
| TIKSKGIAV | HLA-B08:01 | COL10A1 | Breast | 1809 |
| YTIKSKGIAV | HLA-B08:01 | COL10A1 | Breast | 1810 |
| VILSKAYPA | HLA-B08:01 | COL10A1 | Breast | 1811 |
| IPFLLLVSL | HLA-B08:01 | COL10A1 | Breast | 1812 |
| HVHVKGTHV | HLA-B08:01 | COL10A1 | Breast | 1813 |
| LPNTKTQF | HLA-B08:01 | COL10A1 | Breast | 1814 |
| TQFFIPYTI | HLA-B13:02 | COL10A1 | Breast | 1815 |
| KTQFFIPYTI | HLA-B13:02 | COL10A1 | Breast | 1816 |
| LPQIPFLLLV | HLA-B13:02 | COL10A1 | Breast | 1817 |
| HSSFSGFLV | HLA-B13:02 | COL10A1 | Breast | 1818 |
| PQIPFLLLV | HLA-B13:02 | COL10A1 | Breast | 1819 |
| GIYYFSYHV | HLA-B13:02 | COL10A1 | Breast | 1820 |
| GLYKNGTPV | HLA-B13:02 | COL10A1 | Breast | 1821 |
| YPAIGTPIPE | HLA-B46:01 | COL10A1 | Breast | 1822 |
| FSGFLVAPM | HLA-B46:01 | COL10A1 | Breast | 1823 |
| YVHSSFSGF | HLA-B46:01 | COL10A1 | Breast | 1824 |
| FTVILSKAY | HLA-B46:01 | COL10A1 | Breast | 1825 |
| ILYNRQQHY | HLA-B46:01 | COL10A1 | Breast | 1826 |
| TIKSKGIAV | HLA-B46:01 | COL10A1 | Breast | 1827 |
| SAIDLTEN | HLA-B46:01 | COL10A1 | Breast | 1828 |
| PAIGTPIPF | HLA-B46:01 | COL10A1 | Breast | 1829 |
| MLPQIPFLL | HLA-C01:02 | COL10A1 | Breast | 1830 |
| FSGFLVAPM | HLA-C01:02 | COL10A1 | Breast | 1831 |
| MLPQIPFLLL | HLA-C01:02 | COL10A1 | Breast | 1832 |
| ILSKAYPAI | HLA-C01:02 | COL10A1 | Breast | 1833 |
| VGPAGAKGM | HLA-C01:02 | COL10A1 | Breast | 1834 |
| IPGPPGPSAV | HLA-C01:02 | COL10A1 | Breast | 1835 |
| FIPYTIKSK | HLA-C01:02 | COL10A1 | Breast | 1836 |
| MLPQIPFL | HLA-C01:02 | COL10A1 | Breast | 1837 |
| FSGFLVAPM | HLA-C03:04 | COL10A1 | Breast | 1838 |
| YPAIGTPIPF | HLA-C03:04 | COL10A1 | Breast | 1839 |
| YVHSSFSGR | HLA-C03:04 | COL10A1 | Breast | 1840 |
| SANQGVTGM | HLA-C03:04 | COL10A1 | Breast | 1841 |
| MPVSAFTVI | HLA-C03:04 | COL10A1 | Breast | 1842 |
| FLLLVSLNL | HLA-C03:04 | COL10A1 | Breast | 1843 |
| KGIPGSHGL | HLA-C03:04 | COL10A1 | Breast | 1844 |
| FGKPGLPGL | HLA-C03:04 | COL10A1 | Breast | 1845 |
| VSAFTVIL | HLA-C03:04 | COL10A1 | Breast | 1846 |
| HYDPRTGIF | HLA-C04:01 | COL10A1 | Breast | 1847 |
| MLPQIPFLL | HLA-C04:01 | COL10A1 | Breast | 1848 |
| QHYDPRTGIF | HLA-C04:01 | COL10A1 | Breast | 1849 |
| TYDEYTKGYL | HLA-C04:01 | COL10A1 | Breast | 1850 |
| HYDPRTGI | HLA-C04:01 | COL10A1 | Breast | 1851 |
| KGDVGPAGL | HLA-C04:01 | COL10A1 | Breast | 1852 |
| YYFSYHVHV | HLA-C07:01 | COL10A1 | Breast | 1853 |
| ERYQMPTGI | HLA-C07:01 | COL10A1 | Breast | 1854 |
| LYKNGTPVM | HLA-C07:01 | COL10A1 | Breast | 1855 |
| HYDPRTGIF | HLA-C07:01 | COL10A1 | Breast | 1856 |
| CQIPGIYYF | HLA-C07:01 | COL10A1 | Breast | 1857 |
| SKGLYSSEY | HLA-C07:01 | COL10A1 | Breast | 1858 |
| SSESGFLVA | HLA-C07:01 | COL10A1 | Breast | 1859 |
| IPFDKILY | HLA-C07:01 | COL10A1 | Breast | 1860 |
| YYFSYHVHV | HLA-C07:02 | COL10A1 | Breast | 1861 |
| HYDPRTGIF | HLA-C07:02 | COL10A1 | Breast | 1862 |
| LYKNGTPVM | HLA-C07:02 | COL10A1 | Breast | 1863 |
| VFYAERYQM | HLA-C07:02 | COL10A1 | Breast | 1864 |
| YKNGTPVMY | HLA-C07:02 | COL10A | Breast | 1865 |
| NGTPVMYTY | HLA-C07:02 | COL10A1 | Breast | 1866 |
| FYAERYQM | HLA-C07:02 | COL10A1 | Breast | 1867 |
| QVDWSRLY | HLA-A01:01 | CSAG1 | Melanoma | 1868 |
| VSSLLACLH | HLA-A01:01 | CSAG1 | Melanoma | 1869 |
| ATTVSSLLA | HLA-A01:01 | CSAG1 | Melanoma | 1870 |
| QVDWSRLYR | HLA-A01:01 | CSAG1 | Melanoma | 1871 |
| YRDIGLVKM | HLA-A01:01 | CSAG1 | Melanoma | 1872 |
| RLYRDTGLV | HLA-A02:01 | CSAG1 | Melanoma | 1873 |
| TTACWPAFTV | HLA-A02:01 | CSAG1 | Melanoma | 1874 |
| TVSSLLACL | HLA-A02:01 | CSAG1 | Melanoma | 1875 |
| SLLACLHCPG | HLA-A02:01 | CSAG1 | Melanoma | 1876 |
| MSATTACWPA | HLA-A02:01 | CSAG1 | Melanoma | 1877 |
| SLLACLHCP | HLA-A02:01 | CSAG1 | Melanoma | 1878 |
| HPLIPGPEAL | HLA-A02:01 | CSAG1 | Melanoma | 1879 |
| SRLYRDTGLV | HLA-A02:01 | CSAG1 | Melanoma | 1880 |
| TACWPAFTV | HLA-A02:01 | CSAG1 | Melanoma | 1881 |
| PLIPGPEAL | HLA-A02:01 | CSAG1 | Melanoma | 1882 |
| VLGEARGDQV | HLA-A02:01 | CSAG1 | Melanoma | 1883 |
| PLIPGPEA | HLA-A02:01 | CSAG1 | Melanoma | 1884 |
| ACWPAFTVL | HLA-A02:01 | CSAG1 | Melanoma | 1885 |
| RLYRDTGL | HLA-A02:01 | CSAG1 | Melanoma | 1886 |
| RLYRDTGLVKM | HLA-A02:01 | CSAG1 | Melanoma | 1887 |
| YRDTGLVKM | HLA-A02:01 | CSAG1 | Melanoma | 1888 |
| RLYRDTGLVK | HLA-A03:01 | CSAG1 | Melanoma | 1889 |
| SRLYRDTGLVK | HLA-A03:01 | CSAG1 | Melanoma | 1890 |
| WSRLYRDTGLVK | HLA-A03:01 | CSAG1 | Melanoma | 1891 |
| PLIPGPEALSK | HLA-A03:01 | CSAG1 | Melanoma | 1892 |
| LIPGPEALSK | HLA-A03:01 | CSAG1 | Melanoma | 1893 |
| FSNNHPSTPK | HLA-A11:01 | CSAG1 | Melanoma | 1894 |
| QVDWSRLYR | HLA-A11:01 | CSAG1 | Melanoma | 1895 |
| RLYRDTGLVK | HLA-A11:01 | CSAG1 | Melanoma | 1896 |
| LIPGPEALSK | HLA-A11:01 | CSAG1 | Melanoma | 1897 |
| TTVSSLLAC | HLA-A11:01 | CSAG1 | Melanoma | 1898 |
| CWPAFTVL | HLA-A24:02 | CSAG1 | Melanoma | 1899 |
| CWPAFTVLG | HLA-A24:02 | CSAG1 | Melanoma | 1900 |
| ATTACWPAF | HLA-A24:02 | CSAG1 | Melanoma | 1901 |
| NHPSTPKRF | HLA-A24:02 | CSAG1 | Melanoma | 1902 |
| PLIPGPEAL | HLA-A24:02 | CSAG1 | Melanoma | 1903 |
| ACWPAFTVL | HLA-A24:02 | CSAG1 | Melanoma | 1904 |
| MSRKPRASS | HLA-A30:01 | CSAG1 | Melanoma | 1905 |
| RGRGKHPLI | HLA-A30:01 | CSAG1 | Melanoma | 1906 |
| RLYRDTGLVK | HLA-A30:01 | CSAG1 | Melanoma | 1907 |
| REKGPVKEV | HLA-A30:01 | CSAG1 | Melanoma | 1908 |
| LYRDTGLVK | HLA-A30:01 | CSAG1 | Melanoma | 1909 |
| PVKEVPGTK | HLA-A30:01 | CSAG1 | Melanoma | 1910 |
| HPSTPKRFPR | HLA-A33:03 | CSAG1 | Melanoma | 1911 |
| QVDWSRLYR | HLA-A33:03 | CSAG1 | Melanoma | 1912 |
| LVKMSRKPR | HLA-A33:03 | CSAG1 | Melanoma | 1913 |
| DTGLVKMSR | HLA-A33:03 | CSAG1 | Melanoma | 1914 |
| DQVDWSRLYR | HLA-A33:03 | CSAG1 | Melanoma | 1915 |
| HPLIPGPEAL | HLA-B07:02 | CSAG1 | Melanoma | 1916 |
| KPRASSPF | HLA-B07:02 | CSAG1 | Melanoma | 1917 |
| FPRQPKREKGPV | HLA-B07:02 | CSAG1 | Melanoma | 1918 |
| ACWPAFTVL | HLA-B07:02 | CSAG1 | Melanoma | 1919 |
| TPKRRQRGKHPL | HLA-B08:01 | CSAG1 | Melanoma | 1920 |
| WSRLYRDTGL | HLA-B08:01 | CSAG1 | Melanoma | 1921 |
| SRKPRASSPF | HLA-B08:01 | CSAG1 | Melanoma | 1922 |
| SATTVSSL | HLA-B08:01 | CSAG1 | Melanoma | 1923 |
| HPLIPGPEAL | HLA-B08:01 | CSAG1 | Melanoma | 1924 |
| PLIPGPEAL | HLA-B08:01 | CSAG1 | Melanoma | 1925 |
| TACWPAFTV | HLA-B13:02 | CSAG1 | Melanoma | 1926 |
| TTACWPAFTV | HLA-B13:02 | CSAG1 | Melanoma | 1927 |
| MSATTACWPA | HLA-B13:02 | CSAG1 | Melanoma | 1928 |
| RLYRDTGLV | HLA-B13:02 | CSAG1 | Melanoma | 1929 |
| REKGPVKEV | HLA-B13:02 | CSAG1 | Melanoma | 1930 |
| ACWPAFTV | HLA-B13:02 | CSAG1 | Melanoma | 1931 |
| MSATTVSSL | HLA-B46:01 | CSAG1 | Melanoma | 1932 |
| MSATTVSSLL | HLA-B46:01 | CSAG1 | Melanoma | 1933 |
| FSNNHPSTPKRF | HLA-B46:01 | CSAG1 | Melanoma | 1934 |
| PLIPGPEAL | HLA-B46:01 | CSAG1 | Melanoma | 1935 |
| FSNNHPSTP | HLA-B46:01 | CSAG1 | Melanoma | 1936 |
| PLIPGPEA | HLA-B46:01 | CSAG1 | Melanoma | 1937 |
| MSATTVSSL | HLA-C01:02 | CSAG1 | Melanoma | 1938 |
| SATTVSSLL | HLA-C01:02 | CSAG1 | Melanoma | 1939 |
| TVSSLLACL | HLA-C01:02 | CSAG1 | Melanoma | 1940 |
| LIPGPEAL | HLA-C01:02 | CSAG1 | Melanoma | 1941 |
| CWPAFTVL | HLA-C01:02 | CSAG1 | Melanoma | 1942 |
| SATTVSSL | HLA-C01:02 | CSAG1 | Melanoma | 1943 |
| MSATTVSSL | HLA-C03:04 | CSAG1 | Melanoma | 1944 |
| SATTVSSLL | HLA-C03:04 | CSAG1 | Melanoma | 1945 |
| MSATTVSSLL | HLA-C03:04 | CSAG1 | Melanoma | 1946 |
| ACWPAFTVL | HLA-C03:04 | CSAG1 | Melanoma | 1947 |
| TACWPAFTVL | HLA-C03:04 | CSAG1 | Melanoma | 1948 |
| YRDTGLVKM | HLA-C04:01 | CSAG1 | Melanoma | 1949 |
| LYRDTGLVKM | HLA-C04:01 | CSAG1 | Melanoma | 1950 |
| MSATTVSSL | HLA-C04:01 | CSAG1 | Melanoma | 1951 |
| CWPAFTVL | HLA-C04:01 | CSAG1 | Melanoma | 1952 |
| LIPGPEAL | HLA-C04:01 | CSAG1 | Melanoma | 1953 |
| YRDTGLVKM | HLA-C07:01 | CSAG1 | Melanoma | 1954 |
| SRLYRDTGL | HLA-C07:01 | CSAG1 | Melanoma | 1955 |
| MSATTVSSL | HLA-C07:01 | CSAG1 | Melanoma | 1956 |
| RRGRGKHPL | HLA-C07:01 | CSAG1 | Melanoma | 1957 |
| TVLGEARGD | HLA-C07:01 | CSAG1 | Melanoma | 1958 |
| YRDTGLVKM | HLA-C07:02 | CSAG1 | Melanoma | 1959 |
| SRLYRDTGL | HLA-C07:02 | CSAG1 | Melanoma | 1960 |
| LYRDTGLVKM | HLA-C07:02 | CSAG1 | Melanoma | 1961 |
| ACWPAFTVL | HLA-C07:02 | CSAG1 | Melanoma | 1962 |
| SRKPRASSP | HLA-C07:02 | CSAG1 | Melanoma | 1963 |
| CSAVFHERY | HLA-A01:01 | CTCFL | Cervical; Ovarian | 1964 |
| YSAAELKCRY | HLA-A01:01 | CTCFL | Cervical; Ovarian | 1965 |
| YCSAVFHERY | HLA-A01:01 | CTCFL | Cervical; Ovarian | 1966 |
| AISIQQELY | HLA-A01:01 | CTCFL | Cervical; Ovarian | 1967 |
| RSDEIVLTV | HLA-A01:01 | CTCFL | Cervical; Ovarian | 1968 |
| PSEESEKY | HLA-A01:01 | CTCFL | Cervical; Ovarian | 1969 |
| ITDARHHAW | HLA-A01:01 | CTCFL | Cervical; Ovarian | 1970 |
| KSDLRFLGL | HLA-A01:01 | CTCFL | Cervical; Ovarian | 1971 |
| FLVEMGFYHV | HLA-A02:01 | CTCFL | Cervical; Ovarian | 1972 |
| MMLVSAWLL | HLA-A02:01 | CTCFL | Cervical; Ovarian | 1973 |
| FLDLKLHGI | HLA-A02:01 | CTCFI | Cervical; Ovarian | 1974 |
| ILWVGNSEV | HLA-A02:01 | CTCFL | Cervical; Ovarian | 1975 |
| ILVEAAVQV | HLA-A02:01 | CTCFL | Cervical; Ovarian | 1976 |
| LFLVEMGFYHV | HLA-A02:01 | CTCFL | Cervical; Ovarian | 1977 |
| LLFIGTIKV | HLA-A02:01 | CTCFL | Cervical; Ovarian | 1978 |
| RMMLVSAWL | HLA-A02:01 | CTCFL | Cervical; Ovarian | 1979 |
| LLFLVEMGFYHV | HLA-A02:01 | CTCFL | Cervical; Ovarian | 1980 |
| RMMLVSAWLL | HLA-A02:01 | CTCFL | Cervical; Ovarian | 1981 |
| GLIPTVLTL | HLA-A02:01 | CTCFL | Cervical; Ovarian | 1982 |
| KLHGILVEA | HLA-A02:01 | CTCFL | Cervical; Ovarian | 1983 |
| FLVEMGFYHVS | HLA-A02:01 | CTCFL | Cervical; Ovarian | 1984 |
| WILWVGNSEV | HLA-A02:01 | CTCFL | Cervical; Ovarian | 1985 |
| KLLFIGTIKV | HLA-A02:01 | CTCFL | Cervical; Ovarian | 1986 |
| SLAETTGLIKL | HLA-A02:01 | CTCFL | Cervical; Ovarian | 1987 |
| VLEEEVELV | HLA-A02:01 | CTCFL | Cervical; Ovarian | 1988 |
| SVLEBEVEL | HLA-A02:01 | CTCFL | Cervical; Ovarian | 1989 |
| WLIVLLFLV | HLA-A02:01 | CTCFL | Cervical; Ovarian | 1990 |
| SVLEEEVELV | HLA-A02:01 | CTCFL | Cervical; Ovarian | 1991 |
| ILQKHGENV | HLA-A02:01 | CTCFL | Cervical; Ovarian | 1992 |
| RMSSFNRHMK | HLA-A03:01 | CTCFL | Cervical; Ovarian | 1993 |
| KQAFYYSYK | HLA-A03:01 | CTCFL | Cervical; Ovarian | 1994 |
| GTMKIHILQK | HLA-A03:01 | CTCFL | Cervical; Ovarian | 1995 |
| VTNSRICYK | HLA-A03:01 | CTCFL | Cervical; Ovarian | 1996 |
| RICYKQAFYY | HLA-A03:01 | CTCFL | Cervical; Ovarian | 1997 |
| GLIPTVLTLK | HLA-A03:01 | CTCFL | Cervical; Ovarian | 1998 |
| ANFIPTVYK | HLA-A03:01 | CTCFL | Cervical; Ovarian | 1999 |
| SLAETTGLIK | HLA-A03:01 | CTCFL | Cervical; Ovarian | 2000 |
| KLLFIGTIK | HLA-A03:01 | CTCFL | Cervical; Ovarian | 2001 |
| SLLQGPLCR | HLA-A03:01 | CTCFL | Cervical; Ovarian | 2002 |
| GTMKIHILQK | HLA-A11:01 | CTCFL | Cervical; Ovarian | 2003 |
| VTNSRICYK | HLA-A11:01 | CTCFL | Cervical; Ovarian | 2004 |
| SVTNSRICYK | HLA-A11:01 | CTCFL | Cervical; Ovarian | 2005 |
| KQAFYYSYK | HLA-A11:01 | CTCFL | Cervical; Ovarian | 2006 |
| SVLSEQFTK | HLA-A11:01 | CTCFL | Cervical; Ovarian | 2007 |
| ANFIPTVYK | HLA-A11:01 | CTCFL | Cervical; Ovarian | 2008 |
| KYILTLQTV | HLA-A24:02 | CTCFL | Cervical; Ovarian | 2009 |
| KYASVEVKPF | HLA-A24:02 | CTCFL | Cervical; Ovarian | 2010 |
| KYILTLQTVHP | HLA-A24:02 | CTCFL | Cervical: Ovarian | 2011 |
| KYQCPHCATI | HLA-A24:02 | CTCFL | Cervical; Ovarian | 2012 |
| KWSGLKPQTF | HLA-A24:02 | CTCFL | Cervical; Ovarian | 2013 |
| IYAGNNMHSL | HLA-A24:02 | CTCFL | Cervical; Ovarian | 2014 |
| LYSPQEMEVLQF | HLA-A24:02 | CTCFL | Cervical; Ovarian | 2015 |
| IYAGNNMHSLL | HLA-A24:02 | CTCFL | Cervical; Ovarian | 2016 |
| RVHMRNLHA | HLA-A30:01 | CTCFL | Cervical; Ovarian | 2017 |
| RTKEQLFFV | HLA-A30:01 | CTCFL | Cervical; Ovarian | 2018 |
| RTHSEATSK | HLA-A30:01 | CTCFL | Cervical; Ovarian | 2019 |
| KQAFYYSYK | HLA-A30:01 | CTCFL | Cervical; Ovarian | 2020 |
| HMKTHTSEK | HLA-A30:01 | CTCFL | Cervical; Ovarian | 2021 |
| STKNQRKTK | HLA-A30:01 | CTCFL | Cervical; Ovarian | 2022 |
| TMKIHILQK | HLA-A30:01 | CTCFL | Cervical; Ovarian | 2023 |
| RFKCKHCSY | HLA-A30:01 | CTCFL | Cervical; Ovarian | 2024 |
| FSRWINLHR | HLA-A33:03 | CTCFL | Cervical; Ovarian | 2025 |
| TFRTVTLLR | HLA-A33:03 | CTCFL | Cervical; Ovarian | 2026 |
| DVCMFTSSR | HLA-A33:03 | CTCFL | Cervical; Ovarian | 2027 |
| SSRMSSFNR | HLA-A33:03 | CTCFL | Cervical; Ovarian | 2028 |
| MFTSSRMSSFNR | HLA-A33:03 | CTCFL | Cervical; Ovarian | 2029 |
| ETYNQGRRR | HLA-A33:03 | CTCFL | Cervical: Ovarian | 2030 |
| DCLQMLQVWQR | HLA-A33:03 | CTCFL | Cervical; Ovarian | 2031 |
| TYNQGRRRR | HLA-A33:03 | CTCFL | Cervical; Ovarian | 2032 |
| EKNQLLAER | HLA-A33:03 | CTCFL | Cervical; Ovarian | 2033 |
| KPHLCHLCL | HLA-B07:02 | CTCFL | Cervical; Ovarian | 2034 |
| HPKAGLGPEDPL | HLA-B07:02 | CTCFL | Cervical; Ovarian | 2035 |
| RPYKCNDCNM | HLA-B07:02 | CTCFL | Cervical; Ovarian | 2036 |
| RPYKCNDCNMAF | HLA-B07:02 | CTCFL | Cervical; Ovarian | 2037 |
| IPRCKFHPDCL | HLA-B07:02 | CTCFL | Cervical; Ovarian | 2038 |
| QPGPGLLWL | HLA-B07:02 | CTCFL | Cervical; Ovarian | 2039 |
| SPQEMEVL | HLA-B07:02 | CTCFL | Cervical; Ovarian | 2040 |
| GPGSGPLL | HLA-B07:02 | CTCFL | Cervical; Ovarian | 2041 |
| GPGSGPLLRL | HLA-B07:02 | CTCFL | Cervical; Ovarian | 2042 |
| MLQVWQRLFPL | HLA-B08:01 | CTCFL | Cervical; Ovarian | 2043 |
| CLKTFRTVTL | HLA-B08:01 | CTCFL | Cervical; Ovarian | 2044 |
| QVWQRLFPL | HLA-B08:01 | CTCFL | Cervical; Ovarian | 2045 |
| ELKCRYCSA | HLA-B08:01 | CTCFL | Cervical; Ovarian | 2046 |
| IPRCKFHPDCL | HLA-B08:01 | CTCFL | Cervical; Ovarian | 2047 |
| CSMCKYASV | HLA-B08:01 | CTCFL | Cervical; Ovarian | 2048 |
| EATSKRSL | HLA-B08:01 | CTCFL | Cervical; Ovarian | 2049 |
| EQFTKIKEL | HLA-B08:01 | CTCFL | Cervical; Ovarian | 2050 |
| DSKLAVSL | HLA-B08:01 | CTCFL | Cervical; Ovarian | 2051 |
| THARKSDL | HLA-B08:01 | CTCFL | Cervical; Ovarian | 2052 |
| DLKLHGIL | HLA-B08:01 | CTCFL | Cervical; Ovarian | 2053 |
| HDANFIPTV | HLA-B13:02 | CTCFL | Cervical; Ovarian | 2054 |
| LQQCVAISI | HLA-B13:02 | CTCFL | Cervical; Ovarian | 2055 |
| KQAFYYSYKI | HLA-B13:02 | CTCFL | Cervical; Ovarian | 2056 |
| QAFYYSYKI | HLA-B13:02 | CTCFL | Cervical; Ovarian | 2057 |
| YHDANFIPTV | HLA-B13:02 | CTCFL | Cervical; Ovarian | 2058 |
| YQCPHCATI | HLA-B13:02 | CTCFL | Cervical; Ovarian | 2059 |
| QQQEGVQVV | HLA-B13:02 | CTCFL | Cervical; Ovarian | 2060 |
| GLIPTVLTL | HLA-B13:02 | CTCFL | Cervical; Ovarian | 2061 |
| RSDEIVLTV | HLA-B13:02 | CTCFL | Cervical; Ovarian | 2062 |
| GFSRWILWV | HLA-B13:02 | CTCFL | Cervical; Ovarian | 2063 |
| FTSSRMSSF | HLA-B46:01 | CTCFL | Cervical; Ovarian | 2064 |
| YASVEVKPF | HLA-B46:01 | CTCFL | Cervical; Ovarian | 2065 |
| YAGNNMHSL | HLA-B46:01 | CTCFL | Cervical; Ovarian | 2066 |
| MAATEISVL | HLA-B46:01 | CTCFL | Cervical; Ovarian | 2067 |
| HDANFIPTVY | HLA-B46:01 | CTCFL | Cervical; Ovarian | 2068 |
| VAISIQQEL | HLA-B46:01 | CTCFL. | Cervical; Ovarian | 2069 |
| KCNDCNMAF | HLA-B46:01 | CTCFL | Cervical; Ovarian | 2070 |
| FSRWITSKW | HLA-B46:01 | CTCFL | Cervical; Ovarian | 2071 |
| RFKCKHCSY | HLA-B46:01 | CTCFL | Cervical; Ovarian | 2072 |
| DANFIPTVY | HLA-B46:01 | CTCFL | Cervical: Ovarian | 2073 |
| FTSSRMSSF | HLA-C01:02 | CTCFL | Cervical; Ovarian | 2074 |
| YAGNNMHSL | HLA-C01:02 | CTCFL | Cervical; Ovarian | 2075 |
| YSPQEMEVL | HLA-C01:02 | CTCFL | Cervical; Ovarian | 2076 |
| MAATEISVL | HLA-C01:02 | CTCFL | Cervical; Ovarian | 2077 |
| SAHRNLCLL | HLA-C01:02 | CTCFL | Cervical; Ovarian | 2078 |
| FTQSGTMKI | HLA-C01:02 | CTCFL | Cervical; Ovarian | 2079 |
| LIPTVLTL | HLA-C01:02 | CTCFL | Cervical; Ovarian | 2080 |
| FHPDCLQML | HLA-C01:02 | CTCFL | Cervical; Ovarian | 2081 |
| GGPGSGPLL | HLA-C01:02 | CTCFL | Cervical; Ovarian | 2082 |
| MAATEISVL | HLA-C03:04 | CTCFL | Cervical; Ovarian | 2083 |
| YAGNNMHSL | HLA-C03:04 | CTCFL | Cervical; Ovarian | 2084 |
| VAISIQQEL | HLA-C03:04 | CTCFL | Cervical; Ovarian | 2085 |
| YASVEASKL | HLA-C03:04 | ICTCFL | Cervical; Ovarian | 2086 |
| YASVEVKPF | HLA-C03:04 | CTCFL | Cervical; Ovarian | 2087 |
| FTSEAVEL | HLA-C03:04 | CTCFL | Cervical; Ovarian | 2088 |
| AATEISVL | HLA-C03:04 | CTCFL | Cervical; Ovarian | 2089 |
| HAYSAAEL | HLA-C03:04 | CTCFL | Cervical; Ovarian | 2090 |
| YHDANFIPTVY | HLA-C04:01 | CTCFL | Cervical; Ovarian | 2091 |
| YHDANFIPTV | HLA-C04:01 | CTCFL | Cervical; Ovarian | 2092 |
| HHAWLIVLL | HLA-C04:01 | CTCFL | Cervical; Ovarian | 2093 |
| VQQPGPGLL | HLA-C04:01 | CTCFL | Cervical; Ovarian | 2094 |
| LHAYSAAEL | HLA-C04:01 | CTCFL | Cervical; Ovarian | 2095 |
| HFTSEAVEL | HLA-C04:01 | CTCFL | Cervical; Ovarian | 2096 |
| VLEEEVEL | HLA-C04:01 | CTCFL | Cervical: Ovarian | 2097 |
| FTSEAVEL | HLA-C04:01 | CTCFL | Cervical; Ovarian | 2098 |
| TFRTVILL | HLA-C04:01 | CTCFL | Cervical; Ovarian | 2099 |
| HAWLIVLL | HLA-C04:01 | CTCFL | Cervical; Ovarian | 2100 |
| TRFTQSGTM | HLA-C07:01 | CTCFL | Cervical; Ovarian | 2101 |
| FRKYHDANF | HLA-C07:01 | CTCFL | Cervical; Ovarian | 2102 |
| MRTHSGVHM | HLA-C07:01 | CTCFL | Cervical; Ovarian | 2103 |
| SRICYKQAR | HLA-C07:01 | CTCFL | Cervical; Ovarian | 2104 |
| FRKYHDANFI | HLA-C07:01 | CTCFL | Cervical; Ovarian | 2105 |
| CRYCSAVF | HLA-C07:01 | CTCFL | Cervical; Ovarian | 2106 |
| RRGSRRVTW | HLA-C07:01 | CTCFL | Cervical; Ovarian | 2107 |
| ERSDEIVL | HLA-C07:01 | CTCFL | Cervical; Ovarian | 2108 |
| HRSPSELEA | HLA-C07:01 | CTCFL | Cervical; Ovarian | 2109 |
| FRKYHDANF | HLA-C07:02 | CTCFL | Cervical; Ovarian | 2110 |
| SRICYKQAF | HLA-C07:02 | CTCFL | Cervical; Ovarian | 2111 |
| FHPDCLQML | HLA-C07:02 | CTCFL | Cervical; Ovarian | 2112 |
| FYYSYKIYA | HLA-C07:02 | CTCFL | Cervical; Ovarian | 2113 |
| VHMRNLHAY | HLA-C07:02 | CTCFL | Cervical; Ovarian | 2114 |
| HHAWLIVLL | HLA-C07:02 | CTCFL | Cervical; Ovarian | 2115 |
| YKQAFYYSY | HLA-C07:02 | CTCFL | Cervical; Ovarian | 2116 |
| ARHHAWLIVL | HLA-C07:02 | CTCFL | Cervical; Ovarian | 2117 |
| FYYSYKIY | HLA-C07:02 | CTCFL | Cervical; Ovarian | 2118 |
| MRTHSGVHM | HLA-C07:02 | CTCFL | Cervical; Ovarian | 2119 |
| HSWPWQISLQY | HLA-A01:01 | CTRC | Pancreas | 2120 |
| YIDWINEKM | HLA-A01:01 | CTRC | Pancreas | 2121 |
| RIDWWGFRV | HLA-A01:01 | CTRC | Pancreas | 2122 |
| LSDTIQVAC | HLA-A01:01 | CTRC | Pancreas | 2123 |
| LLPKDYPCYV | HLA-A02:01 | CTRC | Pancreas | 2124 |
| KLQQGLQPV | HLA-A02:01 | CTRC | Pancreas | 2125 |
| SLLPKDYPCYV | HLA-A02:01 | CTRC | Pancreas | 2126 |
| TLIASNFVL | HLA-A02:01 | CTRC | Pancreas | 2127 |
| MLGITVLAAL | HLA-A02:01 | CTRC | Pancreas | 2128 |
| CQLENGSWEV | HLA-A02:01 | CTRC | Pancreas | 2129 |
| ALIKLAEHV | HLA-A02:01 | CTRC | Pancreas | 2130 |
| LLLRNDIAL | HLA-A02:01 | CTRC | Pancreas | 2131 |
| DSLLPKDYPCYV | HLA-A02:01 | CTRC | Pancreas | 2132 |
| KLAEHVEL | HLA-A02:01 | CTRC | Pancreas | 2133 |
| SLFVGVDTIHV | HLA-A02:01 | CTRC | Pancreas | 2134 |
| GLQPVVDHA | HLA-A02:01 | CTRC | Pancreas | 2135 |
| SLFVGVDTI | HLA-A02:01 | CTRC | Pancreas | 2136 |
| KLAEHVELS | HLA-A02:01 | CTRC | Pancreas | 2137 |
| RTYRVAVGK | HLA-A03:01 | CTRC | Pancreas | 2138 |
| LLRNDIALIK | HLA-A03:01 | CTRC | Pancreas | 2139 |
| TRTYRVAVGK | HLA-A03:01 | CTRC | Pancreas | 2140 |
| RLWINGPIADK | HLA-A03:01 | CTRC | Pancreas | 2141 |
| VVYTRVSAY | HLA-A03:01 | CTRC | Pancreas | 2142 |
| RTYRVAVGK | HLA-A11:01 | CTRC | Pancreas | 2143 |
| GVDTIHVHK | HLA-A11:01 | CTRC | Pancreas | 2144 |
| TTWRWKTKK | HLA-A11:01 | CTRC | Pancreas | 2145 |
| SAYIDWINEK | HLA-A11:01 | CTRC | Pancreas | 2146 |
| AYIDWINEK | HLA-A11:01 | CTRC | Pancreas | 2147 |
| SRIDWWGFRVK | HLA-A11:01 | CTRC | Pancreas | 2148 |
| VYTRVSAYI | HLA-A24:02 | CTRC | Pancreas | 2149 |
| CYVTGWGRLW | HLA-A24:02 | CTRC | Pancreas | 2150 |
| KDYPCYVTGW | HLA-A24:02 | CTRC | Pancreas | 2151 |
| AYIDWINEK | HLA-A24:02 | CTRC | Pancreas | 2152 |
| VWPWERTTW | HLA-A24:02 | CTRC | Pancreas | 1215 |
| RVKKTMVCA | HLA-A30:01 | CTRC | Pancreas | 2154 |
| RTYRVAVGK | HLA-A30:01 | CTRC | Pancreas | 2155 |
| RTTWRWKTK | HLA-A30:01 | CTRC | Pancreas | 2156 |
| AYIDWINEK | HLA-A30:01 | CTRC | Pancreas | 2157 |
| CISNTRTYR | HLA-A33:03 | CTRC | Pancreas | 2158 |
| CSRIDWWGFR | HLA-A33:03 | CTRC | Pancreas | 2159 |
| HCISNTRTYR | HLA-A33:03 | CTRC | Pancreas | 2160 |
| EVFGIVSFGSR | HLA-A33:03 | CTRC | Pancreas | 2161 |
| WVWTPSTSTR | HLA-A33:03 | CTRC | Pancreas | 2162 |
| DYPCYVTGWGR | HLA-A33:03 | CTRC | Pancreas | 2163 |
| MPSCCAMIL | HLA-B07:02 | CTRC | Pancreas | 2164 |
| LPSSSLQSM | HLA-B07:02 | CTRC | Pancreas | 2165 |
| RPHSWPWQISL | HLA-B07:02 | CTRC | Pancreas | 2166 |
| RPHSWPWQI | HLA-B07:02 | CTRC | Pancreas | 2167 |
| HVHKRWNAL | HLA-B08:01 | CTRC | Pancreas | 2168 |
| IHVHKRWNAL | HLA-B08:01 | CTRC | Pancreas | 2169 |
| HVHKRWNALL | HLA-B08:01 | CTRC | Pancreas | 2170 |
| WINEKMQL | HLA-B08:01 | CTRC | Pancreas | 2171 |
| CLPEKDSL | HLA-B08:01 | CTRC | Pancreas | 2172 |
| AEHVELSDTI | HLA-B13:02 | CTRC | Pancreas | 2173 |
| CQLENGSWEV | HLA-B13:02 | CTRC | Pancreas | 2174 |
| VELSDTIQV | HLA-B13:02 | CTRC | Pancreas | 2175 |
| RIDWWGFRV | HLA-B13:02 | CTRC | Pancreas | 2176 |
| SLFVGVDTI | HLA-B13:02 | CTRC | Pancreas | 2177 |
| RPHSWPWQI | HLA-B13:02 | CTRC | Pancreas | 2178 |
| VVYTRVSAY | HLA-B46:01 | CTRC | Pancreas | 2179 |
| ASSCGVPSF | HLA-B46:01 | CTRC | Pancreas | 2180 |
| HSWPWQISL | HLA-B46:01 | CTRC | Pancreas | 2181 |
| CSRIDWWGF | HLA-B46:01 | CTRC | Pancreas | 2182 |
| HCISNTRTY | HLA-B46:01 | CTRC | Pancreas | 2183 |
| HSWPWQISL | HLA-C01:02 | CTRC | Pancreas | 2184 |
| AMILPSSSL | HLA-C01:02 | CTRC | Pancreas | 2185 |
| YIDWINEKM | HLA-C01:02 | CTRC | Pancreas | 2186 |
| ILPSSSLQSM | HLA-C01:02 | CTRC | Pancreas | 2187 |
| CLPEKDSLL | HLA-C01:02 | CTRC | Pancreas | 2188 |
| CLPEKDSL | HLA-C01:02 | CTRC | Pancreas | 2189 |
| HSWPWQISL | HLA-C03:04 | CTRC | Pancreas | 2190 |
| LGITVLAAL | HLA-C03:04 | CTRC | Pancreas | 2191 |
| AMILPSSSL | HLA-C03:04 | CTRC | Pancreas | 2192 |
| CATPGPTVW | HLA-C03:04 | CTRC | Pancreas | 2193 |
| MILPSSSL | HLA-C03:04 | CTRC | Pancreas | 2194 |
| YIDWINEKM | HLA-C04:01 | CTRC | Pancreas | 2195 |
| MPSCCAMIL | HLA-C04:01 | CTRC | Pancreas | 2196 |
| RHTCGGTLI | HLA-C04:01 | CTRC | Pancreas | 2197 |
| SWEVFGIVSF | HLA-C04:01 | CTRC | Pancreas | 2198 |
| VEDEEGSLP | HLA-C04:01 | CTRC | Pancreas | 2199 |
| VEDEEGSL | HLA-C04:01 | CTRC | Pancreas | 2200 |
| WRHTCGGTL | HLA-C07:01 | CTRC | Pancreas | 2201 |
| HKRWNALLL | HLA-C07:01 | CTRC | Pancreas | 2202 |
| WRHTCGGTLI | HLA-C07:01 | CTRC | Pancreas | 2203 |
| HSWPWQISL | HLA-C07:01 | CTRC | Pancreas | 2204 |
| TRKKPVVY | HLA-C07:01 | CTRC | Pancreas | 2205 |
| TRKKPVVYT | HLA-C07:01 | CTRC | Pancreas | 2206 |
| WRHTQGGTL | HLA-C07:02 | CTRC | Pancreas | 2207 |
| HKRWNALLL | HLA-C07:02 | CTRC | Pancreas | 2208 |
| VHKRWNALL | HLA-C07:02 | CTRC | Pancreas | 2209 |
| TRKKPVVY | HLA-C07:02 | CTRC | Pancreas | 2210 |
| ARPHSWPWQI | HLA-C07:02 | CTRC | Pancreas | 2211 |
| KADIYTQNFY | HLA-A01:01 | CYP11A1 | Adrenal Gland | 2212 |
| TSMTLQWHLY | HLA-A01:01 | CYP11A1 | Adrenal Gland | 2213 |
| TTSMTLQWHLY | HLA-A01:01 | CYP11A1 | Adrenal Gland | 2214 |
| HHDYRGILY | HLA-A01:01 | CYP11A1 | Adrenal Gland | 2215 |
| YMIPAKTLV | HLA-A02:01 | CYP11A1 | Adrenal Gland | 2216 |
| FAFESITNV | HLA-A02:01 | CYP11A1 | Adrenal Gland | 2217 |
| TMLQLVPLL | HLA-A02:01 | CYP11A1 | Adrenal Gland | 2218 |
| FLINMLENFRV | HLA-A02:01 | CYP11A1 | Adrenal Gland | 2219 |
| YMIPAKTLVQV | HLA-A02:01 | CYP11A1 | Adrenal Gland | 2220 |
| MLAKGLPPRSV | HLA-A02:01 | CYP11A1 | Adrenal Gland | 2221 |
| VLRDYMIPA | HLA-A02:01 | CYP11A1 | Adrenal Gland | 2222 |
| YQMFHTSVPM | HLA-A02:01 | CYP11A1 | Adrenal Gland | 2223 |
| KLGNVESVYV | HLA-A02:01 | CYP11A1 | Adrenal Gland | 2224 |
| RLHPISVTL | HLA-A02:01 | CYP11A1 | Adrenal Gland | 2225 |
| GLPPRSVLV | HLA-A02:01 | CYP11A1 | Adrenal Gland | 2226 |
| NLPPDLFRL | HLA-A02:01 | CYP11A1 | Adrenal Gland | 2227 |
| FLIPPWVAY | HLA-A02:01 | CYP11A1 | Adrenal Gland | 2228 |
| RLFRTKTWK | HLA-A03:01 | CYP11A1 | Adrenal Gland | 2229 |
| MLQLVPLLK | HLA-A03:01 | CYP11A1 | Adrenal Gland | 2230 |
| ILYRLLGDSK | HLA-A03:01 | CYP11A1 | Adrenal Gland | 2231 |
| FLIPPWVAY | HLA-A03:01 | CYP11A1 | Adrenal Gland | 2232 |
| GLPPRSVLVK | HLA-A03:01 | CYP11A1 | Adrenal Gland | 2233 |
| AAWDVIFSK | HLA-A11:01 | CYP11A1 | Adrenal Gland | 2234 |
| ATMLQLVPLLK | HLA-A11:01 | CYP11A1 | Adrenal Gland | 2235 |
| SVLHRRIKK | HLA-A11:01 | CYP11A1 | Adrenal Gland | 2236 |
| MLQLVPLLK | HLA-A11:01 | CYP11A1 | Adrenal Gland | 2237 |
| HVAAWDVIFSK | HLA-A11:01 | CYP11A1 | Adrenal Gland | 2238 |
| RFIDAIYQMF | HLA-A24:02 | CYP11A1 | Adrenal Gland | 2239 |
| IYTQNFYWEL | HLA-A24:02 | CYP11A1 | Adrenal Gland | 2240 |
| GWLNLYHFW | HLA-A24:02 | CYP11A1 | Adrenal Gland | 2241 |
| IYALGREPTF | HLA-A24:02 | CYP11A1 | Adrenal Gland | 2242 |
| TFFFDPENF | HLA-A24:02 | CYP11A1 | Adrenal Gland | 2243 |
| ATKNFLPLL | HLA-A30:01 | CYP11A1 | Adrenal Gland | 2244 |
| STRSPRPFN | HLA-A30:01 | CYP11A1 | Adrenal Gland | 2245 |
| RLFRTKTWK | HLA-A30:01 | CYP11A1 | Adrenal Gland | 2246 |
| KYGPIYREK | HLA-A30:01 | CYP11A1 | Adrenal Gland | 2247 |
| NFYWELRQK | HLA-A30:01 | CYP11A1 | Adrenal Gland | 2248 |
| AAWDVIPSK | HLA-A30:01 | CYP11A1 | Adrenal Gland | 2249 |
| DFVSVLHRR | HLA-A33:03 | CYP11A1 | Adrenal Gland | 2250 |
| YTQNFYWELR | HLA-A33:03 | CYP11A1 | Adrenal Gland | 2251 |
| VAYHQYYQR | HLA-A33:03 | CYP11A1 | Adrenal Gland | 2252 |
| EVVNPEAQR | HLA-A33:03 | CYP11A1 | Adrenal Gland | 2253 |
| ESITNVIFGER | HLA-A33:03 | CYP11A1 | Adrenal Gland | 2254 |
| APREGLGRL | HLA-B07:02 | CYP11A1 | Adrenal Gland | 2255 |
| SAPREGLGRL | HLA-B07:02 | CYP11A1 | Adrenal Gland | 2256 |
| VPMLNLPPDL | HLA-B07:02 | CYP11A1 | Adrenal Gland | 2257 |
| SPQDNGWLNL | HLA-B07:02 | CYP11A1 | Adrenal Gland | 2258 |
| IPSPGDNGWL | HLA-B07:02 | CYP11A1 | Adrenal Gland | 2259 |
| TLRLHPISV | HLA-B08:01 | CYP11A1 | Adrenal Gland | 2260 |
| MATMLQLVPL | HLA-B08:01 | CYP11A1 | Adrenal Gland | 2261 |
| TLQWHLYEM | HLA-B08:01 | CYP11A1 | Adrenal Gland | 2262 |
| TIFLINML | HLA-B08:01 | CYP11A1 | Adrenal Gland | 2263 |
| YMIPAKTL | HLA-B08:01 | CYP11A1 | Adrenal Gland | 2264 |
| AWKKDRVAL | HLA-B08:01 | CYP11A1 | Adrenal Gland | 2265 |
| AELEMTIFLI | HLA-B13:02 | CYP11A1 | Adrenal Gland | 2266 |
| LEMTIFLI | HLA-B13:02 | CYP11A1 | Adrenal Gland | 2267 |
| RNLGFGWGV | HLA-B13:02 | CYP11A1 | Adrenal Gland | 2268 |
| RLHPISVTL | HLA-B13:02 | CYP11A1 | Adrenal Gland | 2269 |
| RQGMLEEVV | HLA-B13:02 | CYP11A1 | Adrenal Gland | 2270 |
| AQGDMATML | HLA-B13:02 | CYP11A1 | Adrenal Gland | 2271 |
| FAFESITNV | HLA-B46:01 | CYP11A1 | Adrenal Gland | 2272 |
| YQMFHTSVPM | HLA-B46:01 | CYP11A1 | Adrenal Gland | 2273 |
| FLIPPWVAY | HLA-B46:01 | CYP11A1 | Adrenal Gland | 2274 |
| LSKDKNITY | HLA-B46:01 | CYP11A1 | Adrenal Gland | 2275 |
| RLHPISVTL | HLA-B46:01 | CYP11A1 | Adrenal Gland | 2276 |
| RLHPISVTL | HLA-C01:02 | CYP11A1 | Adrenal Gland | 2277 |
| LMPEKPISF | HLA-C01:02 | CYP11A1 | Adrenal Gland | 2278 |
| YTQNFYWEL | HLA-C01:02 | CYP11A1 | Adrenal Gland | 2279 |
| MAPEATKNFL | HLA-C01:02 | CYP11A1 | Adrenal Gland | 2280 |
| MAPEATKNF | HLA-C01:02 | CYP11A1 | Adrenal Gland | 2281 |
| RSPRPFNEI | HLA-C01:02 | CYP11A1 | Adrenal Gland | 2282 |
| FAFESITNV | HLA-C03:04 | CYP11A1 | Adrenal Gland | 2283 |
| YALGREPTF | HLA-C03:04 | CYP11A1 | Adrenal Gland | 2284 |
| FAFESITNVI | HLA-C03:04 | CYP11A1 | Adrenal Gland | 2285 |
| YTQNFYWEL | HLA-C03:04 | CYP11A1 | Adrenal Gland | 2286 |
| YVIDPEDVAL | HLA-C03:04 | CYP11A1 | Adrenal Gland | 2287 |
| YYQRPIGVL | HLA-C04:01 | CYP11A1 | Adrenal Gland | 2288 |
| FIDATYQMF | HLA-C04:01 | CYP11A1 | Adrenal Gland | 2289 |
| HHDYRGILY | HLA-C04:01 | CYP11A1 | Adrenal Gland | 2290 |
| NFDPTRWL | HLA-C04:01 | CYP11A1 | Adrenal Gland | 2291 |
| FFDPENFD | HLA-C04:01 | CYP11A1 | Adrenal Gland | 2292 |
| HWCKWPSMI | HLA-C04:01 | CYP11A1 | Adrenal Gland | 2293 |
| YRGILYRLL | HLA-C07:01 | CYP11A1 | Adrenal Gland | 2294 |
| YRLLGDSKM | HLA-C07:01 | CYP11A1 | Adrenal Gland | 2295 |
| YYQRPIGVL | HLA-C07:01 | CYP11A1 | Adrenal Gland | 2296 |
| SRDFVSVL | HLA-C07:01 | CYP11A1 | Adrenal Gland | 2297 |
| VSRDFVSVL | HLA-C07:01 | CYP11A1 | Adrenal Gland | 2298 |
| YYQRPIGVL | HLA-C07:02 | CYP11A1 | Adrenal Gland | 2299 |
| YRGILYRLL | HLA-C07:02 | CYP11A1 | Adrenal Gland | 2300 |
| RFIDAIYQM | HLA-C07:02 | CYP11A1 | Adrenal Gland | 2301 |
| FRVEIQHL | HLA-C07:02 | CYP11A1 | Adrenal Gland | 2302 |
| SSDLVLQNY | HLA-A01:01 | CYP11B2; | Adrenal Gland | 2303 |
| | | CYP11B1 | | |
| SMDAPTSRLY | HLA-A01:01 | CYP11B1 | Adrenal Gland | 2304 |
| LTQEDIKMVY | HLA-A01:01 | CYP11B2; | Adrenal Gland | 2305 |
| | | CYP11B1 | | |
| ASSDLVLQNY | HLA-A01:01 | CYP11B1 | Adrenal Gland | 2306 |
| TLDVQPSIFHY | HLA-A01:01 | CYP11B2; | Adrenal Gland | 2307 |
| | | CYP11B1 | | |
| WLDIRGSGRNFY | HLA-A01:01 | CYP11B1 | Adrenal Gland | 2308 |
| FILRPSMFPL | HLA-A02:01 | CYP11B1 | Adrenal Gland | 2309 |
| FPLLMTLFEL | HLA-A02:01 | CYP11B2; | Adrenal Gland | 2310 |
| | | CYP11B1 | | |
| RLAEAEMLL | HLA-A02:01 | CYP11B2; | Adrenal Gland | 2311 |
| | | CYP11B1 | | |
| RLYPVGLFL | HLA-A02:01 | CYP11B2; | Adrenal Gland | 2312 |
| | | CYP11B1 | | |
| SMFPLLTFRA | HLA-A02:01 | CYP11B1 | Adrenal Gland | 2313 |
| LLMTLFELA | HLA-A02:01 | CYP11B2; | Adrenal Gland | 2314 |
| | | CYP11B1 | | |
| LLMILFEL | HLA-A02:01 | CYP11B2; | Adrenal Gland | 2315 |
| | | CYP11B1 | | |
| MLPEDVEKL | HLA-A02:01 | CYP11B2; | Adrenal Gland | 2316 |
| | | CYP11B1 | | |
| SIFHYTIEA | HLA-A02:01 | CYP11B2; | Adrenal Gland | 2317 |
| | | CYP11B1 | | |
| ILRPSMFPL | HLA-A02:01 | CYP11B1 | Adrenal Gland | 2318 |
| ALFGERLGL | HLA-A02:01 | CYP11B2; | Adrenal Gland | 2319 |
| | | CYP11B1 | | |
| RLAEAEMLLL | HLA-A02:01 | CYP11B2; | Adrenal Gland | 2320 |
| | | CYP11B1 | | |
| ALFGERLGLV | HLA-A02:01 | CYP11B2; | Adrenal Gland | 2321 |
| | | CYP11B1 | | |
| SLHPHRMSL | HLA-A02:01 | CYP11B1 | Adrenal Gland | 2322 |
| TLFELARNPNV | HLA-A02:01 | CYP11B1 | Adrenal Gland | 2323 |
| RLAEAEMLLLL | HLA-A02:01 | CYP11B2; | Adrenal Gland | 2324 |
| | | CYP11B1 | | |
| TVFPLLMTL | HLA-A02:01 | CYP11B1 | Adrenal Gland | 2325 |
| RSLSRWTSPK | HLA-A03:01 | CYP11B1 | Adrenal Gland | 2326 |
| SLSRWTSPK | HLA-A03:01 | CYP11B1 | Adrenal Gland | 2327 |
| FLHALEVMFK | HLA-A03:01 | CYP11B2; | Adrenal Gland | 2328 |
| | | CYP11B1 | | |
| SMFPLLTFR | HLA-A03:01 | CYP11B1 | Adrenal Gland | 2329 |
| LMFMPRSLSR | HLA-A03:01 | CYP11B2; | Adrenal Gland | 2330 |
| | | CYP11B1 | | |
| RLYPVGLFL | HLA-A03:01 | CYP11B2; | Adrenal Gland | 2331 |
| | | CYP11B1 | | |
| RLYPVGLFLER | HLA-A03:01 | CYP11B2; | Adrenal Gland | 2332 |
| | | CYP11B1 | | |
| ALFPRPERY | HLA-A03:01 | CYP11B2; | Adrenal Gland | 2333 |
| | | CYP11B1 | | |
| RVFLYSLGR | HLA-A03:01 | CYP11B1 | Adrenal Gland | 2334 |
| SLSRWTSPK | HLA-A11:01 | CYP11B1 | Adrenal Gland | 2335 |
| SMFPLLTFR | ILA-A11:01 | CYP11B1 | Adrenal Gland | 2336 |
| RSLSRWTSPK | HLA-A11:01 | CYP11B1 | Adrenal Gland | 2337 |
| RVFLYSLGR | HLA-A11:0] | CYP11B1 | Adrenal Gland | 2338 |
| ATTELPLLR | HLA-A11:01 | CYP11B2; | Adrenal Gland | 2339 |
| | | CYP11B1 | | |
| STGSPIQLR | HLA-A11:01 | CYP11B1 | Adrenal Gland | 2340 |
| QTFQELGPIFR | HLA-A11:01 | CYP11B2; | Adrenal Gland | 2341 |
| | | CYP11B1 | | |
| ASISEHPQK | HLA-A11:01 | CYP11B2; | Adrenal Gland | 2342 |
| | | CYP11B1 | | |
| RWLRLLQIW | HLA-A24:02 | CYP11B2; | Adrenal Gland | 2343 |
| | | CYP11B1 | | |
| RYNPQRWLDI | HLA-A24:02 | CYP11B2; | Adrenal Gland | 2344 |
| | | CYP11B1 | | |
| VFPLLMTLF | HLA-A24:02 | CYP11B1 | Adrenal Gland | 2345 |
| RNFYHVPFGF | HLA-A24:02 | CYP11B1 | Adrenal Gland | 2346 |
| MFKSTVQLMF | HLA-A24:02 | CYP11B2; | Adrenal Gland | 2347 |
| | | CYP11B1 | | |
| QYTSIVAEL | HLA-A24:02 | CYP11B1 | Adrenal Gland | 2348 |
| TFQELGPIF | HLA-A24:02 | CYP11B2; | Adrenal Gland | 2349 |
| | | CYP11B1 | | |
| NFYHVPFGF | HLA-A24:02 | CYP11B1 | Adrenal Gland | 2350 |
| RSRHSASFG | HLA-A30:01 | CYP11B1 | Adrenal Gland | 2351 |
| RSLSRWTSPK | HLA-A30:01 | CYP11B1 | Adrenal Gland | 2352 |
| RAKAEVCMA | HLA-A30:01 | CYP11B1 | Adrenal Gland | 2353 |
| AYRQHRGHK | HLA-A30:01 | CYP11B2; | Adrenal Gland | 2354 |
| | | CYP11B1 | | |
| ASISEHPQK | HLA-A30:01 | CYP11B2; | Adrenal Gland | 2355 |
| | | CYP11B1 | | |
| QYGDNCIQK | HLA-A30:01 | CYP11B2; | Adrenal Gland | 2356 |
| | | CYP11B1 | | |
| ALKETLRLY | HLA-A30:01 | CYP11B2; | Adrenal Gland | 2357 |
| | | CYP11B1 | | |
| RWTSPKVWK | HLA-A30:01 | CYP11B1 | Adrenal Gland | 2358 |
| SMFPLLTFR | HLA-A33:03 | CYP11B1 | Adrenal Gland | 2359 |
| MSLEPWVAYR | HLA-A33:03 | CYP11B1 | Adrenal Gland | 2360 |
| HISASFGRWGR | HLA-A33:03 | CYP11B1 | Adrenal Gland | 2361 |
| SASFGRWGR | HLA-A33:03 | CYP11B1 | Adrenal Gland | 2362 |
| MFMPRSLSR | HLA-A33:03 | CYP11B2; | Adrenal Gland | 2363 |
| | | CYP11B1 | | |
| EVLSPNAVQR | HLA-A33:03 | CYP11B1 | Adrenal Gland | 2364 |
| ELGPIFRSR | HLA-A33:03 | CYP11B1 | Adrenal Gland | 2365 |
| STGSPIQLR | HLA-A33:03 | CYP11B1 | Adrenal Gland | 2366 |
| MPRRPGNRWL | HLA-B07:02 | CYP11B1 | Adrenal Gland | 2367 |
| HPQKATTEL | HLA-B07:02 | CYP11B2; | Adrenal Gland | 2368 |
| | | CYP11B1 | | |
| SPIQLRTLSM | HLA-B07:02 | CYP11B1 | Adrenal Gland | 2369 |
| RPQQYTSIV | HLA-B07:02 | CYP11B1 | Adrenal Gland | 2370 |
| SPSSASLNF | HLA-B07:02 | CYP11B2; | Adrenal Gland | 2371 |
| | | CYP11B1 | | |
| RPSMFPLLTF | HLA-B07:02 | CYP11B1 | Adrenal Gland | 2372 |
| SLHPHRMSL | HLA-B08:01 | CYP11B1 | Adrenal Gland | 2373 |
| NAVQRFLPM | HLA-B08:01 | CYP11B1 | Adrenal Gland | 2374 |
| MLLLLHHVL | HLA-B08:01 | CYP11B2; | Adrenal Gland | 2375 |
| | | CYP11B1 | | |
| WLSLQRAQAL | HLA-B08:01 | CYP11B1 | Adrenal Gland | 2376 |
| DIKMVYSF | HLA-B08:01 | CYP11B2; | Adrenal Gland | 2377 |
| | | CYP11B1 | | |
| SQALKKKVL | HLA-B08:01 | CYP11B2; | Adrenal Gland | 2378 |
| | | CYP11B1 | | |
| QALKKKVL | HLA-B08:01 | CYP11B2; | Adrenal Gland | 2379 |
| | | CYP11B1 | | |
| SLNFLHAL | HLA-B08:01 | CYP11B2; | Adrenal Gland | 2380 |
| | | CYP11B1 | | |
| AEAEMLLLL | HLA-B13:02 | CYP11B2; | Adrenal Gland | 2381 |
| | | CYP11B1 | | |
| LNFLHALEV | HLA-B13:02 | CYP11B2; | Adrenal Gland | 2382 |
| | | CYP11B1 | | |
| KEHFEAWDCI | HLA-B13:02 | CYP11B2; | Adrenal Gland | 2383 |
| | | CYP11B1 | | |
| AEMLLLLHHV | HLA-B13:02 | CYP11B2; | Adrenal Gland | 2384 |
| | | CYP11B1 | | |
| RLYPVGLFL | HLA-B13:02 | CYP11B2; | Adrenal Gland | 2385 |
| | | CYP11B1 | | |
| YGDNCIQKI | HLA-B13:02 | CYP11B2; | Adrenal Gland | 2386 |
| | | CYP11B1 | | |
| GQDSEALKY | HLA-B13:02 | CYP11B1 | Adrenal Gland | 2387 |
| RQHRGHKC | HLA-B13:02 | CYP11B2; | Adrenal Gland | 2388 |
| | | CYP11B1 | | |
| LAFSRPQQY | HLA-B46:01 | CYP11B1 | Adrenal Gland | 2389 |
| YSFILRPSM | HLA-B46:01 | CYP11B1 | Adrenal Gland | 2390 |
| YTIEASNLAL | HLA-B46:01 | CYP11B2; | Adrenal Gland | 2391 |
| | | CYP11B1 | | |
| MSLEPWVAY | HLA-B46:01 | CYP11B1 | Adrenal Gland | 2392 |
| VARDESQAL | HLA-B46:01 | CYP11B2; | Adrenal Gland | 2393 |
| | | CYP11B1 | | |
| SLHPHRMSL | HLA-B46:01 | CYP11B1 | Adrenal Gland | 2394 |
| AARVPRTVL | HLA-B46:01 | CYP11B1 | Adrenal Gland | 2395 |
| SLHPHRMSL | HLA-C01:02 | CYP11B1 | Adrenal Gland | 2396 |
| SMDAPTSRL | HLA-C01:02 | CYP11B1 | Adrenal Gland | 2397 |
| YSLGRNPAL | HLA-C01:02 | CYP11B1 | Adrenal Gland | 2398 |
| RVPRTVLPF | HLA-C01:02 | CYP11B1 | Adrenal Gland | 2399 |
| NGPEWRFNRL | HLA-C01:02 | CYP11B2; | Adrenal Gland | 2400 |
| | | CYP11B1 | | |
| HSPSSASL | HLA-C01:02 | CYP11B2; | Adrenal Gland | 2401 |
| | | CYP11B1 | | |
| MLPEDVEKL | HLA-C01:02 | CYP11B2; | Adrenal Gland | 2402 |
| | | CYP11B1 | | |
| ELPLLRAAL | HLA-C01:02 | CYP11B2; | Adrenal Gland | 2403 |
| | | CYP11B1 | | |
| YSLGRNPAL | HLA-C03:04 | CYP11B1 | Adrenal Gland | 2404 |
| YSFILRPSM | HLA-C03:04 | CYP11B1 | Adrenal Gland | 2405 |
| NAVQRFLPM | HLA-C03:04 | CYP11B1 | Adrenal Gland | 2406 |
| YTIEASNLAL | HLA-C03:04 | CYP11B2; | Adrenal Gland | 2407 |
| | | CYP11B1 | | |
| VARDFSQAL | HLA-C03:04 | CYP11B2; | Adrenal Gland | 2408 |
| | | CYP11B1 | | |
| MAVPWLSL | HLA-C03:04 | CYP11B1 | Adrenal Gland | 2409 |
| SLHPHRMSL | HLA-C03:04 | CYP11B1 | Adrenal Gland | 2410 |
| RYDLGGAGM | HLA-C04:01 | CYP11B1 | Adrenal Gland | 2411 |
| KYDLGGAGM | HLA-C04:01 | CYP11B1 | Adrenal Gland | 2412 |
| FRYDLGGAGM | HLA-C04:01 | CYP11B1 | Adrenal Gland | 2413 |
| SMDAPTSRL | HLA-C04:01 | CYP11B1 | Adrenal Gland | 2414 |
| TLDVQPSIF | HLA-C04:01 | CYP11B2; | Adrenal Gland | 2415 |
| | | CYP11B1 | | |
| VFPLLMTLF | HLA-C04:01 | CYP11B1 | Adrenal Gland | 2416 |
| FRSRHSASF | HLA-C07:01 | CYP11B1 | Adrenal Gland | 2417 |
| MRQCLGRRL | HLA-C07:01 | CYP11B2; | Adrenal Gland | 2418 |
| | | CYP11B1 | | |
| ERYNPQRWL | HLA-C07:01 | CYP11B2; | Adrenal Gland | 2419 |
| | | CYP11B1 | | |
| SRLYPVGLFL | HLA-C07:01 | CYP11B1 | Adrenal Gland | 2420 |
| RYNPQRWL | HLA-C07:01 | CYP11B2; | Adrenal Gland | 2421 |
| | | CYP11B1 | | |
| RRPGNRWL | HLA-C07:01 | CYP11B1 | Adrenal Gland | 2422 |
| CRNGPEWRF | HLA-C07:01 | CYP11B1 | Adrenal Gland | 2423 |
| FRSRHSASF | HLA-C07:02 | CYP11B1 | Adrenal Gland | 2424 |
| LRPSMPPLL | HLA-C07:02 | CYP11B1 | Adrenal Gland | 2425 |
| MFKSTVQLM | HLA-C07:02 | CYP11B2; | Adrenal Gland | 2426 |
| | | CYP11B1 | | |
| MRQCLGRRL | HLA-C07:02 | CYP11B2; | Adrenal Gland | 2427 |
| | | CYP11B1 | | |
| FKSTVQLMF | HLA-C07:02 | CYP11B2; | Adrenal Gland | 2428 |
| | | CYP11B1 | | |
| ARVPRTVL | HLA-C07:02 | CYP11B1 | Adrenal Gland | 2429 |
| NYHIPAGVL | HLA-C07:02 | CYP11B1 | Adrenal Gland | 2430 |
| VSSDLVLQNY | HLA-A01:01 | CYP11B2 | Adrenal Gland | 2431 |
| GTLVQVFLY | HLA-A01:01 | CYP11B2 | Adrenal Gland | 2432 |
| VVSSDLVLQNY | HLA-A01:01 | CYP11B2 | Adrenal Gland | 2433 |
| AGTLVQVFLY | HLA-A01:01 | CYP11B2 | Adrenal Gland | 2434 |
| ILEPWVAY | HLA-A01:01 | CYP11B2 | Adrenal Gland | 2435 |
| YTGIVAELL | HLA-A01:01 | CYP11B2 | Adrenal Gland | 2436 |
| SLSRWISPKV | HLA-A02:01 | CYP11B2 | Adrenal Gland | 2437 |
| FILRPGTSPL | HLA-A02:01 | CYP11B2 | Adrenal Gland | 2438 |
| RMILEPWVA | HLA-A02:01 | CYP11B2 | Adrenal Gland | 2439 |
| LLLKAELSL | HLA-A02:01 | CYP11B2 | Adrenal Gland | 2440 |
| FLVETLTQEDI | HLA-A02:01 | CYP11B2 | Adrenal Gland | 2441 |
| LLHHVLKHFL | HLA-A02:01 | CYP11B2 | Adrenal Gland | 2442 |
| FLYSLGRNA | HLA-A02:01 | CYP11B2 | Adrenal Gland | 2443 |
| TLVQVFLYSL | HLA-A02:01 | CYP11B2 | Adrenal Gland | 2444 |
| AFPLLMTLFEL | HLA-A02:01 | CYP11B2 | Adrenal Gland | 2445 |
| FLVETLTQE | HLA-A02:01 | CYP11B2 | Adrenal Gland | 2446 |
| IPAGTLVQV | HLA-A02:01 | CYP11B2 | Adrenal Gland | 2447 |
| TLFELARNPDV | HLA-A02:01 | CYP11B2 | Adrenal Gland | 2448 |
| IVAELLLKA | HLA-A02:01 | CYP11B2 | Adrenal Gland | 2449 |
| ILRPGTSPLL | HLA-A02:01 | CYP11B2 | Adrenal Gland | 2450 |
| FLYSLGRNAA | HLA-A02:01 | CYP11B2 | Adrenal Gland | 2451 |
| RSLSRWISPK | HLA-A03:01 | CYP11B2 | Adrenal Gland | 2452 |
| SLSRWISPK | HLA-A03:01 | CYP11B2 | Adrenal Gland | 2453 |
| RLNPDVLSPK | HLA-A03:01 | CYP11B2 | Adrenal Gland | 2454 |
| MILEPWVAY | HLA-A03:01 | CYP11B2 | Adrenal Gland | 2455 |
| SLSRWISPK | HLA-A11:01 | CYP11B2 | Adrenal Gland | 2456 |
| RSLSRWISPK | HLA-A11:01 | CYP11B2 | Adrenal Gland | 2457 |
| GTSPLLTFR | HLA-A11:01 | CYP11B2 | Adrenal Gland | 2458 |
| GIVAELLLK | HLA-A11:01 | CYP11B2 | Adrenal Gland | 2459 |
| RWISPKVWK | HLA-A11:01 | CYP11B2 | Adrenal Gland | 2460 |
| RNFHHVPFGF | HLA-A24:02 | CYP11B2 | Adrenal Gland | 2461 |
| LYSLGRNAALF | HLA-A24:02 | CYP11B2 | Adrenal Gland | 2462 |
| AFPLLMTLF | HLA-A24:02 | CYP11B2 | Adrenal Gland | 2463 |
| HYTGIVAEL | HLA-A24:02 | CYP11B2 | Adrenal Gland | 2464 |
| NFHHVPPGF | HLA-A24:02 | CYP11B2 | Adrenal Gland | 2465 |
| RARALGTRA | HLA-A30:01 | CYP11B2 | Adrenal Gland | 2466 |
| RSLSRWISPK | HLA-A30:01 | CYP11B2 | Adrenal Gland | 2467 |
| RARALGTRAA | HLA-A30:01 | CYP11B2 | Adrenal Gland | 2468 |
| RWISPKVWK | HLA-A30:01 | CYP11B2 | Adrenal Gland | 2469 |
| LSRWISPKV | HLA-A30:01 | CYP11B2 | Adrenal Gland | 2470 |
| MILEPWVAYR | HLA-A33:03 | CYP11B2 | Adrenal Gland | 2471 |
| GTRAARAPR | HLA-A33:03 | CYP11B2 | Adrenal Gland | 2472 |
| YSLGRNAALFPR | HLA-A33:03 | CYP11B2 | Adrenal Gland | 2473 |
| DVLSPKAVQR | HLA-A33:03 | CYP11B2 | Adrenal Gland | 2474 |
| IYQELAFNR | HLA-A33:03 | CYP11B2 | Adrenal Gland | 2475 |
| SPLLTFRAI | HLA-B07:02 | CYP11B2 | Adrenal Gland | 2476 |
| RPQHYTGIV | HLA-B07:02 | CYP11B2 | Adrenal Gland | 2477 |
| AARAPRTVL | HLA-B07:02 | CYP11B2 | Adrenal Gland | 2478 |
| GPRMVCVML | HLA-B07:02 | CYP11B2 | Adrenal Gland | 2479 |
| SPKAVQRFL | HLA-B07:02 | CYP11B2 | Adrenal Gland | 2480 |
| WLSLQRARAL | HLA-B08:01 | CYP11B2 | Adrenal Gland | 2481 |
| KAVQRFLPM | HLA-B08:01 | CYP11B2 | Adrenal Gland | 2482 |
| SPKAVQRFLPM | HLA-B08:01 | CYP11B2 | Adrenal Gland | 2483 |
| ELLLKAEL | HLA-B08:01 | CYP11B2 | Adrenal Gland | 2484 |
| HHVLKHEL | HLA-B08:01 | CYP11B2 | Adrenal Gland | 2485 |
| HPGNRWLRL | HLA-B08:01 | CYP11B2 | Adrenal Gland | 2486 |
| LERVVSSDLV | HLA-B13:02 | CYP11B2 | Adrenal Gland | 2487 |
| LEMHQTFQEL | HLA-B13:02 | CYP11B2 | Adrenal Gland | 2488 |
| VDTTAFPLL | HLA-B13:02 | CYP11B2 | Adrenal Gland | 2489 |
| EQGYEHLHL | HLA-B13:02 | CYP11B2 | Adrenal Gland | 2490 |
| ARNPDVQQI | HLA-B13:02 | CYP11B2 | Adrenal Gland | 2491 |
| RNPDVQQI | HLA-B13:02 | CYP11B2 | Adrenal Gland | 2492 |
| KAVQRFLPM | HLA-B46:01 | CYP11B2 | Adrenal Gland | 2493 |
| LAFNRPQHY | HLA-B46:01 | CYP11B2 | Adrenal Gland | 2494 |
| RAPRTVLPF | HLA-B46:01 | CYP11B2 | Adrenal Gland | 2495 |
| MILEPWVAY | HLA-B46:01 | CYP11B2 | Adrenal Gland | 2496 |
| ILEPWVAY | HLA-B46:01 | CYP11B2 | Adrenal Gland | 2497 |
| ILRPGTSPL | HLA-B46:01 | CYP11B2 | Adrenal Gland | 2498 |
| YSLGRNAAL | HLA-C01:02 | CYP11B2 | Adrenal Gland | 2499 |
| ILRPGTSPL | HLA-C01:02 | CYP11B2 | Adrenal Gland | 2500 |
| RAPRTVLPF | HLA-C01:02 | CYP11B2 | Adrenal Gland | 2501 |
| LRPGTSPL | HLA-C01:02 | CYP11B2 | Adrenal Gland | 2502 |
| AFPLLMTL | HLA-C01:02 | CYP11B2 | Adrenal Gland | 2503 |
| YSLGRNAAL | HLA-C03:04 | CYP11B2 | Adrenal Gland | 2504 |
| TAFPLLMTL | HLA-C03:04 | CYP11B2 | Adrenal Gland | 2505 |
| KAVQRFLPM | HLA-C03:04 | CYP11B2 | Adrenal Gland | 2506 |
| VAAPWLSL | HLA-C03:04 | CYP11B2 | Adrenal Gland | 2507 |
| RYNLGGPRM | HLA-C04:01 | CYP11B2 | Adrenal Gland | 2508 |
| GYEHLHLEM | HLA-C04:01 | CYP11B2 | Adrenal Gland | 2509 |
| HYTGIVAEL | HLA-C04:01 | CYP11B2 | Adrenal Gland | 2510 |
| AFPLLMTLF | HLA-C04:01 | CYP11B2 | Adrenal Gland | 2511 |
| AFPLLMTL | HLA-C04:01 | CYP11B2 | Adrenal Gland | 2512 |
| FRYNLGGPRM | HLA-C07:01 | CYP11B2 | Adrenal Gland | 2513 |
| LRPGTSPLL | HLA-C07:01 | CYP11B2 | Adrenal Gland | 2514 |
| FRYNLGGPRMV | HLA-C07:01 | CYP11B2 | Adrenal Gland | 2515 |
| RNPDVQQIL | HLA-C07:01 | CYP11B2 | Adrenal Gland | 2516 |
| ARNPDVQQI | HLA-C07:01 | CYP11B2 | Adrenal Gland | 2517 |
| ARAPRTVL | HLA-C07:01 | CYP11B2 | Adrenal Gland | 2518 |
| HYTGIVAEL | HLA-C07:02 | CYP11B2 | Adrenal Gland | 2519 |
| LRPGTSPLL | HLA-C07:02 | CYP11B2 | Adrenal Gland | 2520 |
| FRYNLGGPRM | HLA-C07:02 | CYP11B2 | Adrenal Gland | 2521 |
| ARAPRTVL | HLA-C07:02 | CYP11B2 | Adrenal Gland | 2522 |
| ARNPDVQQIL | HLA-C07:02 | CYP11B2 | Adrenal Gland | 2523 |
| MAVREDLYCFSY | HLA-A01:01 | DCAF4L2 | Liver | 2524 |
| WSCAWSLSIHAY | HLA-A01:01 | DCAF4L2 | Liver | 2525 |
| SSDVLAQQF | HLA-A01:01 | DCAF4L2 | Liver | 2526 |
| LMAVREDLY | HLA-A01:01 | DCAF4L2 | Liver | 2527 |
| QVEAGGSKY | HLA-A01:01 | DCAF4L2 | Liver | 2528 |
| HLDSHLLLC | HLA-A01:01 | DCAF4L2 | Liver | 2529 |
| LTTPELRVY | HLA-A01:01 | DCAF4L2 | Liver | 2530 |
| ILQDGQFLV | HLA-A02:01 | DCAF4L2 | Liver | 2531 |
| VLLPASLFI | HLA-A02:01 | DCAF4L2 | Liver | 2532 |
| RVYPHKTLYV | HLA-A02:01 | DCAF4L2 | Liver | 2533 |
| AVLLPASLFI | HLA-A02:01 | DCAF4L2 | Liver | 2534 |
| SMCWASLNHL | HLA-A02:01 | DCAF4L2 | Liver | 2535 |
| QILQDGQFLV | HLA-A02:01 | DCAF4L2 | Liver | 2536 |
| SLFIGSFPGM | HLA-A02:01 | DCAF4L2 | Liver | 2537 |
| FLRFANYCRI | HLA-A02:01 | DCAF4L2 | Liver | 2538 |
| QQFAIMTPL | HLA-A02:01 | DCAF4L2 | Liver | 2539 |
| GLADTPSCAVLL | HLA-A02:01 | DCAF4L2 | Liver | 2540 |
| GLADTPSCAV | HLA-A02:01 | DCAF4L2 | Liver | 2541 |
| GLADTPSCA | HLA-A02:01 | DCAF4L2 | Liver | 2542 |
| GLTTPELRV | HLA-A02:01 | DCAF4L2 | Liver | 2543 |
| TLYVPNRKV | HLA-A02:01 | DCAF4L2 | Liver | 2544 |
| HLLLCFVGL | HLA-A02:01 | DCAF4L2 | Liver | 2545 |
| SLNHLDSHLL | HLA-A02:01 | DCAF4L2 | Liver | 2546 |
| GLNAPSMLRK | HLA-A03:01 | DCAF4L2 | Liver | 2547 |
| RVYPHKTLY | HLA-A03:01 | DCAF4L2 | Liver | 2548 |
| KLWDLRATK | HLA-A03:01 | DCAF4L2 | Liver | 2549 |
| KTLYVPNRKT | HLA-A11:01 | DCAF4L2 | Liver | 2550 |
| GLNAPSMLRK | HLA-A11:01 | DCAF4L2 | Liver | 2551 |
| RVYPHKTLY | HLA-A11:01 | DCAF4L2 | Liver | 2552 |
| GTIKLWDLR | HLA-A11:01 | DCAF4L2 | Liver | 2553 |
| SSLASDRFNR | HLA-A11:01 | DCAF4L2 | Liver | 2554 |
| QFAIMTPLLF | HLA-A24:02 | DCAF4L2 | Liver | 2555 |
| KYGIITMRQL | HLA-A24:02 | DCAF4L2 | Liver | 2556 |
| AWSCAWSLSI | HLA-A24:02 | DCAF4L2 | Liver | 2557 |
| AYLPVHVNE | HLA-A24:02 | DCAF4L2 | Liver | 2558 |
| VYPHKTLYV | HLA-A24:02 | DCAF4L2 | Liver | 2559 |
| RVYPHKTLYV | HLA-A30:01 | DCAF4L2 | Liver | 2560 |
| ASDRFNRILA | HLA-A30:01 | DCAF4L2 | Liver | 2561 |
| RVYPHKTLY | HLA-A30:01 | DCAF4L2 | Liver | 2562 |
| KLWDLRATK | HLA-A30:01 | DCAF4L2 | Liver | 2563 |
| GSKYGIIM | HLA-A30:01 | DCAF4L2 | Liver | 2564 |
| RVSCMQRKK | HLA-A30:01 | DCAF4L2 | Liver | 2565 |
| FANYCRIAR | HLA-A33:03 | DCAF4L2 | Liver | 2566 |
| FLRFANYCR | HLA-A33:03 | DCAF4L2 | Liver | 2567 |
| CYTRIWSLR | HLA-A33:03 | DCAF4L2 | Liver | 2568 |
| SKYGIITMR | HLA-A33:03 | DCAF4L2 | Liver | 2569 |
| ELRVSCMQR | HLA-A33:03 | DCAF4L2 | Liver | 2570 |
| FPGMRRPGM | HLA-B07:02 | DCAF4L2 | Liver | 2571 |
| APSMLRKNQL | HLA-B07:02 | DCAF4L2 | Liver | 2572 |
| VPNRKVNSM | HLA-B07:02 | DCAF4L2 | Liver | 2573 |
| LPASLFIGSF | HLA-B07:02 | DCAF4L2 | Liver | 2574 |
| FPGMRRPGML | HLA-B08:01 | DCAF4L2 | Liver | 2575 |
| FPGMRRPGM | HLA-B08:01 | DCAF4L2 | Liver | 2576 |
| CMQRKKVQI | HLA-B08:01 | DCAF4L2 | Liver | 2577 |
| DAWSCAWSL | HLA-B08:01 | DCAF4L2 | Liver | 2578 |
| TGTIKLWDL | HLA-B08:01 | DCAF4L2 | Liver | 2579 |
| PSMLRKNQL | HLA-B08:01 | DCAF4L2 | Liver | 2580 |
| WSCAWSLSI | HLA-B13:02 | DCAF4L2 | Liver | 2581 |
| LDSHLLLCFV | HLA-B13:02 | DCAF4L2 | Liver | 2582 |
| QQFAIMTPLL | HLA-B13:02 | DCAF4L2 | Liver | 2583 |
| SQQVLLTNV | HLA-B13:02 | DCAF4L2 | Liver | 2584 |
| GLTTPELRV | HLA-B13:02 | DCAF4L2 | Liver | 2585 |
| TLYVPNRKV | HLA-B13:02 | DCAF4L2 | Liver | 2586 |
| FAIMTPLLF | HLA-B46:01 | DCAF4L2 | Liver | 2587 |
| LSIHAYHSF | HLA-B46:01 | DCAF4L2 | Liver | 2588 |
| LLTTIPSPY | HLA-B46:01 | DCAF4L2 | Liver | 2589 |
| FQIPDAWSC | HLA-B46:01 | DCAF4L2 | Liver | 2590 |
| SIHAYHSF | HLA-B46:01 | DCAF4L2 | Liver | 2591 |
| WSLRHGHLL | HLA-C01:02 | DCAF4L2 | Liver | 2592 |
| FAIMTPLLF | HLA-C01:02 | DCAF4L2 | Liver | 2593 |
| LADTPSCAV | HLA-C01:02 | DCAF4L2 | Liver | 2594 |
| YVPNRKVNSM | HLA-C01:02 | DCAF4L2 | Liver | 2595 |
| YLPVHVNEE | HLA-C01:02 | DCAF4L2 | Liver | 2596 |
| LSHDSAVTSL | HLA-C01:02 | DCAF4L2 | Liver | 2597 |
| FAIMTPLLF | HLA-C03:04 | DCAF4L2 | Liver | 2598 |
| SAVTSLQIL | HLA-C03:04 | DCAF4L2 | Liver | 2599 |
| LANTNTDQL | HLA-C03:04 | DCAF4L2 | Liver | 2600 |
| FAIMTPLL | HLA-C03:04 | DCAF4L2 | Liver | 2601 |
| FANYCRIAREL | HLA-C03:04 | DCAF4L2 | Liver | 2602 |
| SHDSAVTSL | HLA-C04:01 | DCAF4L2 | Liver | 2603 |
| FRGAPGLLM | HLA-C04:01 | DCAF4L2 | Liver | 2604 |
| FAIMTPLLF | HLA-C04:01 | DCAF4L2 | Liver | 2605 |
| LWDLRATKC | HLA-C04:01 | DCAF4L2 | Liver | 2606 |
| HLDSHLLL | HLA-C04:01 | DCAF4L2 | Liver | 2607 |
| FRGAPGLLM | HLA-C07:01 | DCAF4L2 | Liver | 2608 |
| LRFANYCRI | HLA-C07:01 | DCAF4L2 | Liver | 2609 |
| LRKNQLGFL | HLA-C07:01 | DCAF4L2 | Liver | 2610 |
| RRPGMLCSF | HLA-C07:01 | DCAF4L2 | Liver | 2611 |
| MRGLTTPEL | HLA-C07:01 | DCAF4L2 | Liver | 2612 |
| CRSGEIFGI | HLA-C07:01 | DCAF4L2 | Liver | 2613 |
| FRGAPGLLM | HLA-C07:02 | DCAF4L2 | Liver | 2614 |
| RRPGMLCSF | HLA-C07:02 | DCAF4L2 | Liver | 2615 |
| LRFANYCRI | HLA-C07:02 | DCAF4L2 | Liver | 2616 |
| NYCRIAREL | HLA-C07:02 | DCAF4L2 | Liver | 2617 |
| FRGAPGLL | HLA-C07:02 | DCAF4L2 | Liver | 2618 |
| YVISAPSIY | HLA-A01:01 | DLL3 | Glioma | 2619 |
| FSGLVCACAPGY | HLA-A01:01 | DLL3 | Glioma | 2620 |
| RSFECTCPRQFY | HLA-A01:01 | DLL3 | Glioma | 2621 |
| CTCPRGFY | HLA-A01:01 | DLL3 | Glioma | 2622 |
| DVDPQGIY | HLA-A01:01 | DLL3 | Glioma | 2623 |
| GADPDSAY | HLA-A01:01 | DLL3 | Glioma | 2624 |
| DVDPQGIYV | HLA-A01:01 | DLL3 | Glioma | 2625 |
| LLFPYPSSI | HLA-A02:01 | DLL3 | Glioma | 2626 |
| YMGARCEFPV | HLA-A02:01 | DLL3 | Glioma | 2627 |
| YLLPPALGL | HLA-A02:01 | DLL3 | Glioma | 2628 |
| YLLPPALGLL | HLA-A02:01 | DLL3 | Glioma | 2629 |
| LLFPYPSSILSV | HLA-A02:01 | DLL3 | Glioma | 2630 |
| RLPCRLFFRV | HLA-A02:01 | DLL3 | Glioma | 2631 |
| LLFPYPSSIL | HLA-A02:01 | DLL3 | Glioma | 2632 |
| ALPDALNNL | HLA-A02:01 | DLL3 | Glioma | 2633 |
| YLLPPALGLLV | HLA-A02:01 | DLL3 | Glioma | 2634 |
| LPLPDGLLQV | HLA-A02:01 | DLL3 | Glioma | 2635 |
| PLPDGLLQV | HLA-A02:01 | DLL3 | Glioma | 2636 |
| GLLSQTVIL | HLA-A02:01 | DLL3 | Glioma | 2637 |
| LLAGTPEPSV | HLA-A02:01 | DLL3 | Glioma | 2638 |
| ALPAAPPGL | HLA-A02:01 | DLL3 | Glioma | 2639 |
| GLLSQTVILA | HLA-A02:01 | DLL3 | Glioma | 2640 |
| RIFFRVCLK | HLA-A03:01 | DLL3 | Glioma | 2641 |
| CRLFFRVCLK | HLA-A03:01 | DLL3 | Glioma | 2642 |
| RLFFRVCLKP | HLA-A03:01 | DLL3 | Glioma | 2643 |
| LPCRLFFRVCLK | HLA-A03:01 | DLL3 | Glioma | 2644 |
| SLLARVAGR | HLA-A03:01 | DLL3 | Glioma | 2645 |
| RLAAGGPWAR | HLA-A03:01 | DLL3 | Glioma | 2646 |
| YLLPPALGL | HLA-A03:01 | DLL3 | Glioma | 2647 |
| RLFFRVCLK | HLA-A11:01 | DLL3 | Glioma | 2648 |
| STSSCLSPR | HLA-A11:01 | DLL3 | Glioma | 2649 |
| RSFECTCPR | HLA-A11:01 | DLL3 | Glioma | 2650 |
| VISAPSIYAR | HLA-A11:01 | DLL3 | Glioma | 2651 |
| GTFSFILET | HLA-A11:01 | DLL3 | Glioma | 2652 |
| GTFSFIIETWR | HLA-A11:01 | DLL3 | Glioma | 2653 |
| YVISAPSIYAR | HLA-A11:01 | DLL3 | Glioma | 2654 |
| IYAREVATPLF | HLA-A24:02 | DLL3 | Glioma | 2655 |
| RYLLPPALGL | HLA-A24:02 | DLL3 | Glioma | 2656 |
| GYMGARCEF | HLA-A24:02 | DLL3 | Glioma | 2657 |
| TFSFILETW | HLA-A24:02 | DLL3 | Glioma | 2658 |
| IYVISAPSI | HLA-A24:02 | DLL3 | Glioma | 2659 |
| AYICHCPPGF | HLA-A24:02 | DLL3 | Glioma | 2660 |
| VPELQIHSF | HLA-A24:02 | DLL3 | Glioma | 2661 |
| RARCEPPAV | HLA-A30:01 | DLL3 | Glioma | 2662 |
| RLFFRVCLK | HLA-A30:01 | DLL3 | Glioma | 2663 |
| RVAGRRRLA | HLA-A30:01 | DLL3 | Glioma | 2664 |
| CTRLCRPRSA | HLA-A30:01 | DLL3 | Glioma | 2665 |
| QTFSFIIET | HLA-A30:01 | DLL3 | Glioma | 2666 |
| STSSCLSPR | HLA-A33:03 | DLL3 | Glioma | 2667 |
| FSFIIETWR | HLA-A33:03 | DLL3 | Glioma | 2668 |
| TFSFIIETWR | HLA-A33:03 | DLL3 | Glioma | 2669 |
| ELRFSYRAR | HLA-A33:03 | DLL3 | Glioma | 2670 |
| YVISAPSIYAR | HLA-A33:03 | DLL3 | Glioma | 2671 |
| ECEAPLVCR | HLA-A33:03 | DLL3 | Glioma | 2672 |
| ISAPSIYAR | HLA-A33:03 | DLL3 | Glioma | 2673 |
| APRSPCSARL | HLA-B07:02 | DLL3 | Glioma | 2674 |
| SPRGPSSAT | HLA-B07:02 | DLL3 | Glioma | 2675 |
| SPRMSGLL | HLA-B07:02 | DLL3 | Glioma | 2676 |
| SPCALGAAL | HLA-B07:02 | DLL3 | Glioma | 2677 |
| QPGAPAPDL | HLA-B07:02 | DLL3 | Glioma | 2678 |
| VAGRRRLAA | HLA-B08:01 | DLL3 | Glioma | 2679 |
| ALRCRCRAGF | HLA-B08:01 | DLL3 | Glioma | 2680 |
| YMGARCEFPV | HLA-B08:01 | DLL3 | Glioma | 2681 |
| YAREVATPL | HLA-B08:01 | DLL3 | Glioma | 2682 |
| TPEPSVHAL | HLA-B08:01 | DLL3 | Glioma | 2683 |
| YPSSILSV | HLA-B08:01 | DLL3 | Glioma | 2684 |
| CLDLGHAL | HLA-B08:01 | DLL3 | Glioma | 2685 |
| VILALIFL | HLA-B08:01 | DLL3 | Glioma | 2686 |
| SQTVILALI | HLA-B13:02 | DLL3 | Glioma | 2687 |
| RDAWPGTFSFII | HLA-B13:02 | DLL3 | Glioma | 2688 |
| WELRFSYRA | HLA-B13:02 | DLL3 | Glioma | 2689 |
| RDAWPGTFSF | HLA-B13:02 | DLL3 | Glioma | 2690 |
| LSQTVILALI | HLA-B13:02 | DLL3 | Glioma | 2691 |
| DQIGGGLCL | HLA-B13:02 | DLL3 | Glioma | 2692 |
| PLPDGLLQV | HLA-B13:02 | DLL3 | Glioma | 2693 |
| ALIFLPQTR | HLA-B13:02 | DLL3 | Glioma | 2694 |
| YVISAPSIY | HLA-B46:01 | DLL3 | Glioma | 2695 |
| YAREVATPL | HLA-B46:01 | DLL3 | Glioma | 2696 |
| YAREVATPLF | HLA-B46:01 | DLL3 | Glioma | 2697 |
| LLFPYPSSI | HLA-B46:01 | DLL3 | Glioma | 2698 |
| DAWPGTFSF | HLA-B46:01 | DLL3 | Glioma | 2699 |
| WARDIQRAG | HLA-B46:01 | DLL3 | Glioma | 2700 |
| FIIETWREE | HLA-B46:01 | DLL3 | Glioma | 2701 |
| LLPPALGLL | HLA-C01:02 | DLL3 | Glioma | 2702 |
| YAREVATPL | HLA-C01:02 | DLL3 | Glioma | 2703 |
| YLLPPALGL | HLA-C01:02 | DLL3 | Glioma | 2704 |
| LLFPYPSSI | HLA-C01:02 | DLL3 | Glioma | 2705 |
| VHPDGASAL | HLA-C01:02 | DLL3 | Glioma | 2706 |
| ALPDALNNL | HLA-C01:02 | DLL3 | Glioma | 2707 |
| SAPSRCGPGL | HLA-C01:02 | DLL3 | Glioma | 2708 |
| TVPVSTSSCL | HLA-C01:02 | DLL3 | Glioma | 2709 |
| YAREVATPL | HLA-C03:04 | DLL3 | Glioma | 2710 |
| VATPLFPPL | HLA-C03:04 | DLL3 | Glioma | 2711 |
| YLLPPALGL | HLA-C03:04 | DLL3 | Glioma | 2712 |
| LLFPYPSSI | HLA-C03:04 | DLL3 | Glioma | 2713 |
| FILETWREEL | HLA-C03:04 | DLL3 | Glioma | 2714 |
| YAHFSGLVC | HLA-C03:04 | DLL3 | Glioma | 2715 |
| FRDAWPGTF | HLA-C04:01 | DLL3 | Glioma | 2716 |
| LFPYPSSIL | HLA-C04:01 | DLL3 | Glioma | 2717 |
| SQDAGSRLL | HLA-C04:01 | DLL3 | Glioma | 2718 |
| LLPPALGLL | HLA-C04:01 | DLL3 | Glioma | 2719 |
| VFELQIHSF | HLA-C04:01 | DLL3 | Glioma | 2720 |
| SFECTCPRGF | HLA-C04:01 | DLL3 | Glioma | 2721 |
| FRDAWPGTF | HLA-C07:01 | DLL3 | Glioma | 2722 |
| TRPAGVFEL | HLA-C07:01 | DLL3 | Glioma | 2723 |
| QRYLLPPAL | HLA-C07:01 | DLL3 | Glioma | 2724 |
| FRVCLKPGL | HLA-C07:01 | DLL3 | Glioma | 2725 |
| ARLPCRLFF | HLA-C07:01 | DLL3 | Glioma | 2726 |
| RPGDPQRYL | HLA-C07:01 | DLL3 | Glioma | 2727 |
| ARGPVYTEQ | HLA-C07:01 | DLL3 | Glioma | 2728 |
| ARGPVYTE | HLA-C07:01 | DLL3 | Glioma | 2729 |
| FRDAWPGTP | HLA-C07:02 | DLL3 | Glioma | 2730 |
| TRPAGVFEL | HLA-C07:02 | DLL3 | Glioma | 2731 |
| ARLPCRLFF | HLA-C07:02 | DLL3 | Glioma | 2732 |
| QRYLLPPAL | HLA-C07:02 | DLL3 | Glioma | 2733 |
| GYMGARCEF | HLA-C07:02 | DLL3 | Glioma | 2734 |
| ARGPVYTEQ | HLA-C07:02 | DLL3 | Glioma | 2735 |
| LRPGDPQRY | HLA-C07:02 | DLL3 | Glioma | 2736 |
| FTDFVRPLNI | HLA-A01:01 | DMRTB | Testis | 2737 |
| GGDFQPSYY | HLA-A01:01 | DMRTB1 | Testis | 2738 |
| FGAEAAGSGY | HLA-A01:01 | DMRTB1 | Testis | 2739 |
| VSEPGGDFQPSY | HLA-A01:01 | DMRTB1 | Testis | 2740 |
| FTDFVRPLN | HLA-A01:01 | DMRTB1 | Testis | 2741 |
| YLPPPPPPL | HLA-A02:01 | DMRTB1 | Testis | 2742 |
| FLPPGYLSA | HLA-A02:01 | DMRTB1 | Testis | 2743 |
| FLPPGYLSAL | HLA-A02:01 | DMRTB1 | Testis | 2744 |
| SMHPYCPFPL | HLA-A02:01 | DMRTB1 | Testis | 2745 |
| YLSALHFL | HLA-A02:0] | DMRTB1 | Testis | 2746 |
| YLDAPPGVPL | HLA-A02:01 | DMRTB1 | Testis | 2747 |
| SLAGPPFGA | HLA-A02:01 | DMRTB1 | Testis | 2748 |
| YLDAPPGV | HLA-A02:01 | DMRTB1 | Testis | 2749 |
| YLISERQKIMAA | HLA-A02:01 | DMRTB1 | Testis | 2750 |
| GYLDAPPGV | HLA-A02:01 | DMRTB1 | Testis | 2751 |
| YLISERQKI | HLA-A02:01 | DMRTB1 | Testis | 2752 |
| KIMAAQKVLK | HLA-A03:01 | DMRTB1 | Testis | 2753 |
| IMAAQKVLK | HLA-A03:01 | DMRTB1 | Testis | 2754 |
| RQKIMAAQKVLK | HLA-A03:01 | DMRTB1 | Testis | 2755 |
| HPYCPFPLGY | HLA-A03:01 | DMRTB1 | Testis | 2756 |
| ALQPVLGGR | HLA-A03:01 | DMRTB1 | Testis | 2757 |
| KIMAAQKVLK | HLA-A11:01 | DMRTB1 | Testis | 2758 |
| IMAAQKVLK | HLA-A11:01 | DMRTB1 | Testis | 2759 |
| AALCAQGPK | HLA-A11:01 | DMRTB1 | Testis | 2760 |
| SSFSLTVLF | HLA-A11:01 | DMRTB1 | Testis | 2761 |
| RTVPGPLFT | HLA-A11:01 | DMRTB1 | Testis | 2762 |
| ACFFEQPPR | HLA-A11:01 | DMRTB1 | Testis | 2763 |
| GYLSALHFL | HLA-A24:02 | DMRTB1 | Testis | 2764 |
| PGYLSALHFL | HLA-A24:02 | DMRTB1 | Testis | 2765 |
| GYLSALHF | HLA-A24:02 | DMRTB1 | Testis | 2766 |
| YYLPPPPPPL | HLA-A24:02 | DMRTB1 | Testis | 2767 |
| PPPPPPSSF | HLA-A24:02 | DMRTB1 | Testis | 2768 |
| RGRNPGPRA | HLA-A30:01 | DMRTB1 | Testis | 2769 |
| RQKIMAAQK | HLA-A30:01 | DMRTB1 | Testis | 2770 |
| RCRNHGFLV | HLA-A30:01 | DMRTB1 | Testis | 2771 |
| RWKQCLCEK | HLA-A30:01 | DMRTB1 | Testis | 2772 |
| KCYLISERQK | HLA-A30:01 | DMRTB1 | Testis | 2773 |
| MVRTPKCSR | HLA-A33:03 | DMRTB1 | Testis | 2774 |
| CFFEQPPRGR | HLA-A33:03 | DMRTB1 | Testis | 2775 |
| KMVRTPKCSR | HLA-A33:03 | DMRTB1 | Testis | 2776 |
| EKCYLISER | HLA-A33:03 | DMRTB1 | Testis | 2777 |
| FFEQPPRGR | HLA-A33:03 | DMRTB1 | Testis | 2778 |
| DADPGPEGR | HLA-A33:03 | DMRTB1 | Testis | 2779 |
| RPMRTVPGPL | HLA-B07:02 | DMRTB1 | Testis | 2780 |
| VPAASLRPL | HLA-B07:02 | DMRTB1 | Testis | 2781 |
| GPLDLRRPM | HLA-B07:02 | DMRTB1 | Testis | 2782 |
| APVPVPAASL | HLA-B07:02 | DMRTB1 | Testis | 2783 |
| GPRALQPVI | HLA-B07:02 | DMRTB1 | Testis | 2784 |
| YLISERQKIMAA | HLA-B08:01 | DMRTB1 | Testis | 2785 |
| SERQKIMAA | HLA-B08:01 | DMRTB1 | Testis | 2786 |
| YLISERQKIM | HLA-B08:01 | DMRTB1 | Testis | 2787 |
| NINPDRAL | HLA-B08:01 | DMRTB1 | Testis | 2788 |
| CLCEKCYL | HLA-B08:01 | DMRTB1 | Testis | 2789 |
| VPVPAASL | HLA-B08:01 | DMRTB1 | Testis | 2790 |
| RNHQFLVPV | HLA-B13:02 | DMRTB1 | Testis | 2791 |
| GDFQPSYYL | HLA-B13:02 | DMRTB1 | Testis | 2792 |
| FTDFVRPLNI | HLA-B13:02 | DMRTB1 | Testis | 2793 |
| YLISERQKI | HLA-B13:02 | DMRTB1 | Testis | 2794 |
| SQYQGGGLV | HLA-B13:02 | DMRTB1 | Testis | 2795 |
| RQKIMAAQKV | HLA-B13:02 | DMRTB1 | Testis | 2796 |
| SSMHPYCPF | HLA-B46:01 | DMRTB1 | Testis | 2797 |
| SSFSLTVLF | HLA-B46:01 | DMRTB1 | Testis | 2798 |
| YLPPPPPPL | HLA-B46:01 | DMRTB1 | Testis | 2799 |
| SMHPYCPF | HLA-B46:01 | DMRTB1 | Testis | 2800 |
| FLPPGYLSA | HLA-B46:01 | DMRTB1 | Testis | 2801 |
| YLPPPPPPL | HLA-C01:02 | DMRTB1 | Testis | 2802 |
| FLPPGYLSAL | HLA-C01:02 | DMRTB1 | Testis | 2803 |
| YLDAPPGVPL | HLA-C01:02 | DMRTB1 | Testis | 2804 |
| YCPFPLGYL | HLA-C01:02 | DMRTB1 | Testis | 2805 |
| AAAPAPVPV | HLA-C03:04 | DMRTB1 | Testis | 2806 |
| AAAAAPAPV | HLA-C03:04 | DMRTB1 | Testis | 2807 |
| RAAVAMPSL | HLA-C03:04 | DMRTB1 | Testis | 2808 |
| SGYPGPLDL | HLA-C03:04 | DMRTB1 | Testis | 2809 |
| YLPPPPPPL | HLA-C03:04 | DMRTB1 | Testis | 2810 |
| YLPPPPPPL | HLA-C04:01 | DMRTB1 | Testis | 2811 |
| YLDAPPGVPL | HLA-C04:01 | DMRTB1 | Testis | 2812 |
| YYLPPPPPPL | HLA-C04:01 | DMRTB1 | Testis | 2813 |
| SFSLTVLF | HLA-C04:01 | DMRTB1 | Testis | 2814 |
| NTDDQDAEV | HLA-C04:01 | DMRTB1 | Testis | 2815 |
| FTDFVRPL | HLA-C04:01 | DMRTB1 | Testis | 2816 |
| FRHVSRSQY | HLA-C07:01 | DMRTB1 | Testis | 2817 |
| MRTVPGPLF | HLA-C07:01 | DMRTB1 | Testis | 2818 |
| GRNPGPRAL | HLA-C07:01 | DMRTB1 | Testis | 2819 |
| SGYPGPLDL | HLA-C07:01 | DMRTB1 | Testis | 2820 |
| CRNHGFLVP | HLA-C07:01 | DMRTB1 | Testis | 2821 |
| FRHVSRSQY | HLA-C07:02 | DMRTB1 | Testis | 2822 |
| MRTVPGPLF | HLA-C07:02 | DMRTB1 | Testis | 2823 |
| LFTDFVRPL | HLA-C07:02 | DMRTB1 | Testis | 2824 |
| GRNPGPRAL | HLA-C07:02 | DMRTB1 | Testis | 2825 |
| YLPPPPPPL | HLA-C07:02 | DMRTB1 | Testis | 2826 |
| ATLEDLQNLY | HLA-A01:01 | EPYC | Ovarian | 2827 |
| TLEDLDNLY | HLA-A01:01 | EPYC | Ovarian | 2828 |
| CTCISTTVY | HLA-A01:01 | EPYC | Ovarian | 2829 |
| TLESINYDSETY | HLA-A01:01 | EPYC | Ovarian | 2830 |
| VTAPTLESINY | HLA-A01:01 | EPYC | Ovarian | 2831 |
| NLYNYENIPV | HLA-A02:01 | EPYC | Ovarian | 2832 |
| TLAGLVLGL | HLA-A02:01 | EPYC | Ovarian | 2833 |
| TLAGLVLGLV | HLA-A02:01 | EPYC | Ovarian | 2834 |
| KTLAGLVLGL | HLA-A02:01 | EPYC | Ovarian | 2835 |
| NLDHIPLPL | HLA-A02:01 | EPYC | Ovarian | 2836 |
| YMCLPRLPV | HLA-A02:01 | EPYC | Ovarian | 2837 |
| YLTDNNLDHI | HLA-A02:01 | EPYC | Ovarian | 2838 |
| GLVIFDAAV | HLA-A02:01 | EPYC | Ovarian | 2839 |
| LLCTCISTTV | HLA-A02:01 | EPYC | Ovarian | 2840 |
| RLDGNPINL | HLA-A02:01 | EPYC | Ovarian | 2841 |
| QLPELPTTL | HLA-A02:01 | EPYC | Ovarian | 2842 |
| SLSDLKRIDL | HLA-A02:01 | EPYC | Ovarian | 2843 |
| KLPQLRELV | HLA-A02:01 | EPYC | Ovarian | 2844 |
| KDMYDLHHLY | HLA-A03:01 | EPYC | Ovarian | 2845 |
| RLDGNPINLSK | HLA-A03:01 | EPYC | Ovarian | 2846 |
| ISNNRLGRK | HLA-A03:01 | EPYC | Ovarian | 2847 |
| HELDAIPPLPK | HLA-A03:01 | EPYC | Ovarian | 2848 |
| ELDAIPPLPK | HLA-A03:01 | EPYC | Ovarian | 2849 |
| TAYFYSRENR | HLA-A11:01 | EPYC | Ovarian | 2850 |
| ATVMPSGNR | HLA-A11:01 | EPYC | Ovarian | 2851 |
| ISNNRLGRK | HLA-A11:01 | EPYC | Ovarian | 2852 |
| LTPPPQPEK | HLA-A11:01 | EPYC | Ovarian | 2853 |
| SRFNRIKKINK | HLA-A11:01 | EPYC | Ovarian | 2854 |
| AYFYSRFNR | HLA-A11:01 | EPYC | Ovarian | 2855 |
| YFYSRFNRI | HLA-A24:02 | EPYC | Ovarian | 2856 |
| AYFYSRFNRI | HLA-A24:02 | EPYC | Ovarian | 2857 |
| TYIRKALEDI | HLA-A24:02 | EPYC | Ovarian | 2858 |
| PELPTTLTF | HLA-A24:02 | EPYC | Ovarian | 2859 |
| MYDLHHLYL | HLA-A24:02 | EPYC | Ovarian | 2860 |
| YSRFNRIKK | HLA-A30:01 | EPYC | Ovarian | 2861 |
| RFNRIKKINK | HLA-A30:01 | EPYC | Ovarian | 2862 |
| FYSRFNRIKK | HLA-A30:01 | EPYC | Ovarian | 2863 |
| NVKNLTYIRK | HLA-A30:01 | EPYC | Ovarian | 2864 |
| TAYFYSRFNR | HLA-A33:03 | EPYC | Ovarian | 2865 |
| NVKNLTYIR | HLA-A33:03 | EPYC | Ovarian | 2866 |
| AYFYSRFNR | HLA-A33:03 | EPYC | Ovarian | 2867 |
| TFIDISNNR | HLA-A33:03 | EPYC | Ovarian | 2868 |
| LPRLPVGSL | HLA-B07:02 | EPYC | Ovarian | 2869 |
| CLPRLPVGSL | HLA-B07:02 | EPYC | Ovarian | 2870 |
| YMCLPRLPVGSL | HLA-B07:02 | EPYC | Ovarian | 2871 |
| MCLPRLPVGSL | HLA-B07:02 | EPYC | Ovarian | 2872 |
| SPQEPEFTGV | HLA-B07:02 | EPYC | Ovarian | 2873 |
| LPRLPVGSLV | HLA-B07:02 | EPYC | Ovarian | 2874 |
| LPQLRELVI | HLA-B08:01 | EPYC | Ovarian | 2875 |
| LTYIRKAL | HLA-B08:01 | EPYC | Ovarian | 2876 |
| YFYSRFNRI | HLA-B08:01 | EPYC | Ovarian | 2877 |
| DMYDLHHL | HLA-B08:01 | EPYC | Ovarian | 2878 |
| LPELPTTL | HLA-B08:01 | EPYC | Ovarian | 2879 |
| LPENLRAL | HLA-B08:01 | EPYC | Ovarian | 2880 |
| HELDAIPPL | HLA-B13:02 | EPYC | Ovarian | 2881 |
| YENIPVDKV | HLA-B13:02 | EPYC | Ovarian | 2882 |
| RELVLRDNKI | HLA-B13:02 | EPYC | Ovarian | 2883 |
| RQLPELPTT | HLA-B13:02 | EPYC | Ovarian | 2884 |
| SRFNRIKKI | HLA-B13:02 | EPYC | Ovarian | 2885 |
| KVEIEIATV | HLA-B13:02 | EPYC | Ovarian | 2886 |
| LQNNNILEM | HLA-B46:01 | JEPYC | Ovarian | 2887 |
| LTSNLISEI | HLA-B46:01 | EPYC | Ovarian | 2888 |
| LSKTPQAYM | HLA-B46:01 | EPYC | Ovarian | 2889 |
| SINYDSETY | HLA-B46:01 | EPYC | Ovarian | 2890 |
| FCNVKNLTY | HLA-B46:01 | EPYC | Ovarian | 2891 |
| LSKTPQAY | HLA-B46:01 | EPYC | Ovarian | 2892 |
| VMPSGNREL | HLA-C01:02 | EPYC | Ovarian | 2893 |
| FIDISNNRL | HLA-C01:02 | EPYC | Ovarian | 2894 |
| KTPQAYMCL | HLA-C01:02 | EPYC | Ovarian | 2895 |
| QLPELPTTL | HLA-C01:02 | EPYC | Ovarian | 2896 |
| FDAAVTAPTL | HLA-C03:04 | EPYC | Ovarian | 2897 |
| LQNNNILEM | HLA-C03:04 | EPYC | Ovarian | 2898 |
| VTAPTLESI | HLA-C03:04 | EPYC | Ovarian | 2899 |
| TVYCDDHEL | HLA-C03:04 | EPYC | Ovarian | 2900 |
| AAVTAPTI | HLA-C03:04 | EPYC | Ovarian | 2901 |
| DAAVTAPTL | HLA-C03:04 | EPYC | Ovarian | 2902 |
| KTLAGLVL | HLA-C03:04 | EPYC | Ovarian | 2903 |
| MYDLHHLYL | HLA-C04:01 | EPYC | Ovarian | 2904 |
| FIDISNNRL | HLA-C04:01 | EPYC | Ovarian | 2905 |
| TYDATLEDL | HLA-C04:01 | EPYC | Ovarian | 2906 |
| IFDAAVTAP | HLA-C04:01 | EPYC | Ovarian | 2907 |
| SRFNRIKKI | HLA-C07:01 | EPYC | Ovarian | 2908 |
| LRDNKIRQL | HLA-C07:01 | EPYC | Ovarian | 2909 |
| FRKLPQLREL | HLA-C07:01 | EPYC | Ovarian | 2910 |
| KRIDLTSNL | HLA-C07:01 | EPYC | Ovarian | 2911 |
| YNYENIPVD | HLA-C07:01 | EPYC | Ovarian | 2912 |
| RDNKIRQL | HLA-C07:01 | EPYC | Ovarian | 2913 |
| MYDLHHLYL | HLA-C07:02 | EPYC | Ovarian | 2914 |
| YFYSRENRI | HLA-C07:02 | EPYC | Ovarian | 2915 |
| LRDNKIRQL | HLA-C07:02 | EPYC | Ovarian | 2916 |
| VYCDDHEL | HLA-C07:02 | EPYC | Ovarian | 2917 |
| KRIDLTSNL | HLA-C07:02 | EPYC | Ovarian | 2918 |
| NKNDFASL | HLA-C07:02 | EPYC | Ovarian | 2919 |
| LTSLTILQLY | HLA-A01:01 | G6PC2 | Pancreas | 2920 |
| IPTHEEHLFY | HLA-A01:01 | G6PC2 | Pancreas | 2921 |
| PTHEEHLFY | HLA-A01:01 | G6PC2 | Pancreas | 2922 |
| LSCRGGNNY | HLA-A01:01 | G6PC2 | Pancreas | 2923 |
| FLFAVGFYL | HLA-A02:01 | G6PC2 | Pancreas | 2924 |
| VLNIDLLWSV | HLA-A02:01 | G6PC2 | Pancreas | 2925 |
| FLFAVGFYLL | HLA-A02:01 | G6PC2 | Pancreas | 2926 |
| RLTWSFLWSV | HLA-A02:01 | G6PC2 | Pancreas | 2927 |
| AMGASCVWYV | HLA-A02:01 | G6PC2 | Pancreas | 2928 |
| FLWSVFWLI | HLA-A02:01 | G6PC2 | Pancreas | 2929 |
| LTWSFLWSV | HLA-A02:01 | G6PC2 | Pancreas | 2930 |
| NLFLFLFAV | HLA-A02:01 | G6PC2 | Pancreas | 2931 |
| RLLCALTSL | HLA-A02:01 | G6PC2 | Pancreas | 2932 |
| FIATHFPHQV | HLA-A02:01 | G6PC2 | Pancreas | 2933 |
| FLWSVFWLIQI | HLA-A02:01 | G6PC2 | Pancreas | 2934 |
| FLWSVFWL | HLA-A02:01 | G6PC2 | Pancreas | 2935 |
| VLFGLGFAI | HLA-A02:01 | G6PC2 | Pancreas | 2936 |
| HLFYVLSFCK | HLA-A03:01 | G6PC2 | Pancreas | 2937 |
| MLMKQSGKK | HLA-A03:01 | G6PC2 | Pancreas | 2938 |
| LLWSVPIAK | HLA-A03:01 | G6PC2 | Pancreas | 2939 |
| VIGDWLNLIFK | HLA-A03:01 | G6PC2 | Pancreas | 2940 |
| LLWSVPIAKK | HLA-A03:01 | G6PC2 | Pancreas | 2941 |
| QTASLGTYLK | HLA-A11:01 | G6PC2 | Pancreas | 2942 |
| HLFYVLSFCK | HLA-A11:01 | G6PC2 | Pancreas | 2943 |
| TASLGTYLK | HLA-A11:01 | G6PC2 | Pancreas | 2944 |
| GVLIIQHLQK | HLA-A11:01 | G6PC2 | Pancreas | 2945 |
| AVIGDWLNLIFK | HLA-A11:01 | G6PC2 | Pancreas | 2946 |
| AVGFYLLLR | HLA-A11:01 | G6PC2 | Pancreas | 2947 |
| TYLKTNLFLF | HLA-A24:02 | G6PC2 | Pancreas | 2948 |
| IYFPLCFQF | HLA-A24:02 | G6PC2 | Pancreas | 2949 |
| TWSFLWSVF | HLA-A24:02 | G6PC2 | Pancreas | 2950 |
| NYTLSFRLL | HLA-A24:02 | G6PC2 | Pancreas | 2951 |
| KDYRAYYTFL | HLA-A30:01 | G6PC2 | Pancreas | 2952 |
| GTKMIWVAV | HLA-A30:01 | G6PC2 | Pancreas | 2953 |
| KTNLFLFLFA | HLA-A30:01 | G6PC2 | Pancreas | 2954 |
| WLNLIFKWK | HLA-A30:01 | G6PC2 | Pancreas | 2955 |
| SVFWLIQISV | HLA-A30:01 | G6PC2 | Pancreas | 2956 |
| IFKWILFGHR | HLA-A33:03 | G6PC2 | Pancreas | 2957 |
| NSEMFLLSCR | HLA-A33:03 | G6PC2 | Pancreas | 2958 |
| FMSNVGDPR | HLA-A33:03 | G6PC2 | Pancreas | 2959 |
| DKFSITLHR | HLA-A33:03 | G6PC2 | Pancreas | 2960 |
| EMFLLSCR | HLA-A33:03 | G6PC2 | Pancreas | 2961 |
| NNYTLSFR | HLA-A33:03 | G6PC2 | Pancreas | 2962 |
| TPGIQTASL | HLA-B07:02 | G6PC2 | Pancreas | 2963 |
| YPNHSSPCL | HLA-B07:02 | G6PC2 | Pancreas | 2964 |
| IPYSVHMLM | HLA-B07:02 | G6PC2 | Pancreas | 2965 |
| HTPGIQTASL | HLA-B07:02 | G6PC2 | Pancreas | 2966 |
| RPYWWVQET | HLA-B07:02 | G6PC2 | Pancreas | 2967 |
| YLLLRVLNI | HLA-B08:01 | G6PC2 | Pancreas | 2968 |
| FLHRNGVL | HLA-B08:01 | G6PC2 | Pancreas | 2969 |
| NLIFKWKSI | HLA-B08:01 | G6PC2 | Pancreas | 2970 |
| NLIFKWIL | HLA-B08:01 | G6PC2 | Pancreas | 2971 |
| NLIFKWILF | HLA-B08:01 | G6PC2 | Pancreas | 2972 |
| IDLLWSVPI | HLA-B13:02 | G6PC2 | Pancreas | 2973 |
| MDFLHRNGVLII | HLA-B13:02 | G6PC2 | Pancreas | 2974 |
| MDFLHRNGVLI | HLA-B13:02 | G6PC2 | Pancreas | 2975 |
| FLWSVFWLI | HLA-B13:02 | G6PC2 | Pancreas | 2976 |
| WLIQISVCI | HLA-B13:02 | G6PC2 | Pancreas | 2977 |
| SIWPCNGRI | HLA-B13:02 | G6PC2 | Pancreas | 2978 |
| WIHIDTTPF | HLA-B46:01 | G6PC2 | Pancreas | 2979 |
| FAINSEMFI | HLA-B46:01 | G6PC2 | Pancreas | 2980 |
| RAYYTFLNF | HLA-B46:01 | G6PC2 | Pancreas | 2981 |
| ISRVFIATH | HLA-B46:01 | G6PC2 | Pancreas | 2982 |
| LQKDYRAY | HLA-B46:01 | G6PC2 | Pancreas | 2983 |
| FIPYSVHML | HLA-C01:02 | G6PC2 | Pancreas | 2984 |
| FAINSEMFL | HLA-C01:02 | G6PC2 | Pancreas | 2985 |
| GMDKFSITL | HLA-C01:02 | G6PC2 | Pancreas | 2986 |
| IWPCNGRIL | HLA-C01:02 | G6PC2 | Pancreas | 2987 |
| HTPGIQTASL | HLA-C01:02 | G6PC2 | Pancreas | 2988 |
| FAINSEMFL | HLA-C03:04 | G6PC2 | Pancreas | 2989 |
| FAVGFYLLL | HLA-C03:04 | G6PC2 | Pancreas | 2990 |
| LSFCKSASI | HLA-C03:04 | G6PC2 | Pancreas | 2991 |
| FAVGFYLL | HLA-C03:04 | G6PC2 | Pancreas | 2992 |
| IGDWLNLIF | HLA-C04:01 | G6PC2 | Pancreas | 2993 |
| VGDPRNIFF | HLA-C04:01 | G6PC2 | Pancreas | 2994 |
| YPNHSSPCL | HLA-C04:01 | G6PC2 | Pancreas | 2995 |
| FFIYFPLCF | HLA-C04:01 | G6PC2 | Pancreas | 2996 |
| AFIPYSVHM | HLA-C04:01 | G6PC2 | Pancreas | 2997 |
| HRHAGGRGL | HLA-C07:01 | G6PC2 | Pancreas | 2998 |
| YRAYYTFLNF | HLA-C07:01 | G6PC2 | Pancreas | 2999 |
| LRVLNIDLL | HLA-C07:01 | G6PC2 | Pancreas | 3000 |
| CRGGNNYTL | HLA-C07:01 | G6PC2 | Pancreas | 3001 |
| NRCPEPHCL | HLA-C07:01 | G6PC2 | Pancreas | 3002 |
| THFPHQVIL | HLA-C07:01 | G6PC2 | Pancreas | 3003 |
| YRAYYTFLNF | HLA-C07:02 | G6PC2 | Pancreas | 3004 |
| AYYTFLNFM | HLA-C07:02 | G6PC2 | Pancreas | 3005 |
| LRVLNIDLL | HLA-C07:02 | G6PC2 | Pancreas | 3006 |
| SRVFIATHF | HLA-C07:02 | G6PC2 | Pancreas | 3007 |
| IYFPLCFQF | HLA-C07:02 | G6PC2 | Pancreas | 3008 |
| NRCPEPHCL | HLA-C07:02 | G6PC2 | Pancreas | 3009 |
| ATDLEKHPY | HLA-A01:01 | HMGB4 | Testis | 3010 |
| TATDLEKHPY | HLA-A01:01 | HMGB4 | Testis | 3011 |
| WSTATDLEKHPY | HLA-A01:01 | HMGB4 | Testis | 3012 |
| YFEELELY | HLA-A01:01 | HMGB4 | Testis | 3013 |
| VSSYVHFLLNY | HLA-A01:01 | HMGB4 | Testis | 3014 |
| YQEEMMNYV | HLA-A02:01 | HMGB4 | Testis | 3015 |
| LLFCQDHYA | HLA-A02:01 | HMGB4 | Testis | 3016 |
| FLLFCQDHYA | HLA-A02:01 | HMGB4 | Testis | 3017 |
| FLLFCQDHYAQL | HLA-A02:01 | HMGB4 | Testis | 3018 |
| KMWSTATDL | HLA-A02:01 | HMGB4 | Testis | 3019 |
| LLFCQDHYAQL | HLA-A02:01 | HMGB4 | Testis | 3020 |
| NVSSYVHFL | HLA-A02:01 | HMGB4 | Testis | 3021 |
| LLRAKYFEEL | HLA-A02:01 | HMGB4 | Testis | 3022 |
| SFLLFCQDHYA | HLA-A02:01 | HMGB4 | Testis | 3023 |
| AKYEALAKL | HLA-A02:01 | HMGB4 | Testis | 3024 |
| FCQDHYAQL | HLA-A02:01 | HMGB4 | Testis | 3025 |
| FLLNYRNKF | HLA-A02:01 | HMGB4 | Testis | 3026 |
| ALAKLDKA | HLA-A02:01 | HMGB4 | Testis | 3027 |
| LLNYRNKFK | HLA-A03:01 | HMGB4 | Testis | 3028 |
| MMNYVGKRK | HLA-A03:01 | HMGB4 | Testis | 3029 |
| MMNYVGKRKK | HLA-A03:01 | HMGB4 | Testis | 3030 |
| SSYVHFLLNY | HLA-A03:01 | HMGB4 | Testis | 3031 |
| RVALLRAK | HLA-A03:01 | HMGB4 | Testis | 3032 |
| RVALLRAKY | HLA-A03:01 | HMGB4 | Testis | 3033 |
| SSYVHFLLNY | HLA-A11:01 | HMGB4 | Testis | 3034 |
| SSYVHFLLNYR | HLA-A11:01 | HMGB4 | Testis | 3035 |
| YVGFKEFSRK | HLA-A11:01 | HMGB4 | Testis | 3036 |
| SYVHFLLNYR | HLA-A11:01 | HMGB4 | Testis | 3037 |
| STATDLEK | HLA-A11:01 | HMGB4 | Testis | 3038 |
| SVVQVAKATGK | HLA-A11:01 | HMGB4 | Testis | 3039 |
| VGFKEFSRK | HLA-A11:01 | HMGB4 | Testis | 3040 |
| RYQEEMMNYV | HLA-A24:02 | HMGB4 | Testis | 3041 |
| HFLLNYRNKF | HLA-A24:02 | HMGB4 | Testis | 3042 |
| RPPSSFLLF | HLA-A24:02 | HMGB4 | Testis | 3043 |
| KYFEELELY | HLA-A24:02 | HMGB4 | Testis | 3044 |
| RRPPSSFLLF | HLA-A24:02 | HMGB4 | Testis | 3045 |
| KAKYEALAK | HLA-A30:01 | HMGB4 | Testis | 3046 |
| SARNRCRGK | HLA-A30:01 | HMGB4 | Testis | 3047 |
| KYRMSARNR | HLA-A30:01 | HMGB4 | Testis | 3048 |
| KARYQEEMMNYV | HLA-A30:01 | HMGB4 | Testis | 3049 |
| SEKWRSISK | HLA-A30:01 | HMGB4 | Testis | 3050 |
| HFLLNYRNK | HLA-A30:01 | HMGB4 | Testis | 3051 |
| YVHFLLNYR | HLA-A33:03 | HMGB4 | Testis | 3052 |
| SYVHFLLNYR | HLA-A33:03 | HMGB4 | Testis | 3053 |
| TYVGFKEFSR | HLA-A33:03 | HMGB4 | Testis | 3054 |
| NYVGKRKKR | HLA-A33:03 | HMGB4 | Testis | 3055 |
| EMMNYVGKR | HLA-A33:03 | HMGB4 | Testis | 3056 |
| HPYEQRVAL | HLA-B07:02 | HMGB4 | Testis | 3057 |
| KHPYEQRVAL | HLA-B07:02 | HMGB4 | Testis | 3058 |
| HPYEQRVALL | HLA-B07:02 | HMGB4 | Testis | 3059 |
| EPRRPPSSF | HLA-B07:02 | HMGB4 | Testis | 3060 |
| RPPSSFLLF | HLA-B07:02 | HMGB4 | Testis | 3061 |
| LLRAKYFEEL | HLA-B08:01 | HMGB4 | Testis | 3062 |
| LLRAKYFEELEL | HLA-B08:01 | HMGB4 | Testis | 3063 |
| HPYEQRVAL | HLA-B08:01 | HMGB4 | Testis | 3064 |
| HEKAKYEAL | HLA-B08:01 | HMGB4 | Testis | 3065 |
| EIQLKPKAN | HLA-B08:01 | HMGB4 | Testis | 3066 |
| RENPNWSVV | HLA-B13:02 | HMGB4 | Testis | 3067 |
| YQEEMMNYV | HLA-B13:02 | HMGB4 | Testis | 3068 |
| KEQQPNTYV | HLA-B13:02 | HMGB4 | Testis | 3069 |
| EKHPYEQRV | HLA-B13:02 | HMGB4 | Testis | 3070 |
| AKYFEELEL | HLA-B13:02 | HMGB4 | Testis | 3071 |
| IQLKPKANV | HLA-B13:02 | HMGB4 | Testis | 3072 |
| SSYVHFLLNY | HLA-B46:01 | HMGB4 | Testis | 3073 |
| FLLNYRNKF | HLA-B46:01 | HMGB4 | Testis | 3074 |
| NTYVGFKEF | HLA-B46:01 | HMGB4 | Testis | 3075 |
| LAKLDKARY | HLA-B46:01 | HMGB4 | Testis | 3076 |
| RPPSSFLLF | HLA-C01:02 | HMGB4 | Testis | 3077 |
| VSSYVHFLL | HLA-C01:02 | HMGB4 | Testis | 3078 |
| KMWSTATDL | HLA-C01:02 | HMGB4 | Testis | 3079 |
| RRPPSSFL | HLA-C01:02 | HMGB4 | Testis | 3080 |
| KHPYEQRVAL | HLA-C01:02 | HMGB4 | Testis | 3081 |
| VSSYVHFLL | HLA-C03:04 | HMGB4 | Testis | 3082 |
| HPYEQRVAL | HLA-C03:04 | HMGB4 | Testis | 3083 |
| FCQDHYAQL | HLA-C03:04 | HMGB4 | Testis | 3084 |
| SSYVHFLL | HLA-C03:04 | HMGB4 | Testis | 3085 |
| RPPSSFLLF | HLA-C04:01 | HMGB4 | Testis | 3086 |
| YQEEMMNYV | HLA-C04:01 | HMGB4 | Testis | 3087 |
| RRPPSSFLL | HLA-C04:01 | HMGB4 | Testis | 3088 |
| YFEELELY | HLA-C04:01 | HMGB4 | Testis | 3089 |
| KYFEELEL | HLA-C04:01 | HMGB4 | Testis | 3090 |
| KYEALAKL | HLA-C04:01 | HMGB4 | Testis | 3091 |
| RRPPSSFLL | HLA-C07:01 | HMGB4 | Testis | 3092 |
| LRAKYFEEL | HLA-C07:01 | HMGB4 | Testis | 3093 |
| NVSSYVHFL | HLA-C07:01 | UMGB4 | Testis | 3094 |
| RRPPSSFL | HLA-C07:01 | HMGB4 | Testis | 3095 |
| RRPPSSFLL | HLA-C07:02 | HMGB4 | Testis | 3096 |
| LRAKYFEEL | HLA-C07:02 | HMGB4 | Testis | 3097 |
| RRPPSSFLLF | HLA-C07:02 | HMGB4 | Testis | 3098 |
| KYFEELEL | HLA-C07:02 | HMGB4 | Testis | 3099 |
| SYVHFLLNY | HLA-C07:02 | HMGB4 | Testis | 3100 |
| STNVGSNTY | HLA-A01:01 | IAPP | Pancreas | 3101 |
| SSTNVGSNTY | HLA-A01:01 | IAPP | Pancreas | 3102 |
| LSSTNVGSNTY | HLA-A01:01 | IAPP | Pancreas | 3103 |
| VLKREPLNY | HLA-A01:01 | IAPP | Pancreas | 3104 |
| FLIVLSVAL | HLA-A02:01 | IAPP | Pancreas | 3105 |
| VLSRNILLEL | HLA-A02:01 | IAPP | Pancreas | 3106 |
| VFLIVLSVAL | HLA-A02:01 | IAPP | Pancreas | 3107 |
| VLSVALNHL | HLA-A02:01 | IAPP | Pancreas | 3108 |
| GILKLQVFL | HLA-A02:01 | IAPP | Pancreas | 3109 |
| KLQVFLIVL | HLA-A02:01 | IAPP | Pancreas | 3110 |
| CLDQIPIFTV | HLA-A02:01 | IAPP | Pancreas | 3111 |
| RQEWIIPV | HLA-A02:01 | IAPP | Pancreas | 3112 |
| MGILKLQVFL | HLA-A02:01 | IAPP | Pancreas | 3113 |
| TVFQENHQV | HLA-A02:01 | IAPP | Pancreas | 3114 |
| RLANFLVHS | HLA-A02:01 | IAPP | Pancreas | 3115 |
| CLDQIPIFT | HLA-A02:01 | IAPP | Pancreas | 3116 |
| VLSVALNHLK | HLA-A03:01 | IAPP | Pancreas | 3117 |
| ILLELRGAK | HLA-A03:01 | IAPP | Pancreas | 3118 |
| IVLSVALNHLK | HLA-A03:01 | IAPP | Pancreas | 3119 |
| TVFQENHQVEK | HLA-A03:01 | IAPP | Pancreas | 3120 |
| IVLSVALNH | HLA-A03:01 | IAPP | Pancreas | 3121 |
| LSVALNHLK | HLA-A11:01 | IAPP | Pancreas | 3122 |
| VLSVALNHLK | HLA-A11:01 | IAPP | Pancreas | 3123 |
| IVLSVALNHLK | HLA-A11:01 | IAPP | Pancreas | 3124 |
| TVFQENHQVEK | HLA-A11:01 | IAPP | Pancreas | 3125 |
| ATLPHVQR | HLA-A11:01 | IAPP | Pancreas | 3126 |
| ATPIESHQVEK | HLA-A11:01 | IAPP | Pancreas | 3127 |
| RWKSGNATL | HLA-A24:02 | IAPP | Pancreas | 3128 |
| VQRSAWQIF | HLA-A24:02 | IAPP | Pancreas | 3129 |
| NFLVHSSNNP | HLA-A24:02 | IAPP | Pancreas | 3130 |
| LDQIPIFTVF | HLA-A24:02 | IAPP | Pancreas | 3131 |
| DQIPIFTVF | HLA-A24:02 | IAPP | Pancreas | 3132 |
| RCLDQIPIF | HLA-A24:02 | IAPP | Pancreas | 3133 |
| KSKVIRWKS | HLA-A30:01 | IAPP | Pancreas | 3134 |
| ATQRLANFLV | HLA-A30:01 | IAPP | Pancreas | 3135 |
| KVIRWKSGNA | HLA-A30:01 | IAPP | Pancreas | 3136 |
| KSKVIRWKSGNA | HLA-A30:01 | IAPP | Pancreas | 3137 |
| KSKVIRWK | HLA-A30:01 | IAPP | Pancreas | 3138 |
| QVFLIVLSV | HLA-A30:01 | IAPP | Pancreas | 3139 |
| RWKSGNATL | HLA-A30:01 | IAPP | Pancreas | 3140 |
| NATLPHVQR | HLA-A33:03 | IAPP | Pancreas | 3141 |
| NTATCATQR | HLA-A33:03 | IAPP | Pancreas | 3142 |
| HLKATPIER | HLA-A33:03 | IAPP | Pancreas | 3143 |
| EWIIPVLSR | HLA-A33:03 | IAPP | Pancreas | 3144 |
| IPVLSRNIL | HLA-B07:02 | IAPP | Pancreas | 3145 |
| IPVLSRNILL | HLA-B07:02 | IAPP | Pancreas | 3146 |
| IIPVLSRNIL | HLA-B07:02 | IAPP | Pancreas | 3147 |
| LPHVQRSAW | HLA-B07:02 | IAPP | Pancreas | 3148 |
| LNHLKATPI | HLA-B08:01 | IAPP | Pancreas | 3149 |
| MGILKLQVF | HLA-B08:01 | IAPP | Pancreas | 3150 |
| MGILKLQVFL | HLA-B08:01 | IAPP | Pancreas | 3151 |
| ILKLQVFLI | HLA-B08:01 | IAPP | Pancreas | 3152 |
| ILKLQVFL | HLA-B08:01 | IAPP | Pancreas | 3153 |
| GILKLQVF | HLA-B08:01 | IAPP | Pancreas | 3154 |
| LDQIPIFTV | HLA-B13:02 | IAPP | Pancreas | 3155 |
| IERQEWIIPV | HLA-B13:02 | IAPP | Pancreas | 3156 |
| TQRLANFLV | HLA-B13:02 | IAPP | Pancreas | 3157 |
| IERCLDQIPI | HLA-B13:02 | IAPP | Pancreas | 3158 |
| RQEWIIPVL | HLA-B13:02 | IAPP | Pancreas | 3159 |
| DQIPIFTV | HLA-B13:02 | IAPP | Pancreas | 3160 |
| HVQRSAWQI | HLA-B13:02 | IAPP | Pancreas | 3161 |
| FLVHSSNNF | HLA-B46:01 | IAPP | Pancreas | 3162 |
| STNVQSNTY | HLA-B46:01 | IAPP | Pancreas | 3163 |
| HSSNNFGAI | HLA-B46:01 | IAPP | Pancreas | 3164 |
| VQRSAWQIF | HLA-B46:01 | IAPP | Pancreas | 3165 |
| FLIVLSVAL | HLA-B46:01 | IAPP | Pancreas | 3166 |
| SSNNFGAIL | HLA-C01:02 | IAPP | Pancreas | 3167 |
| LSRNILLEL | HLA-C01:02 | IAPP | Pancreas | 3168 |
| HSSNNFGAI | HLA-C01:02 | IAPP | Pancreas | 3169 |
| IIPVLSRNI | HLA-C01:02 | IAPP | Pancreas | 3170 |
| REPLNYLPL | HLA-C01:02 | IAPP | Pancreas | 3171 |
| TLPHVQRSAW | HLA-C01:02 | IAPP | Pancreas | 3172 |
| VLSVALNHL | HLA-C01:02 | IAPP | Pancreas | 3173 |
| HSSNNFGAI | HLA-C03:04 | IAPP | Pancreas | 3174 |
| SSNNFGAIL | HLA-C03:04 | IAPP | Pancreas | 3175 |
| FLIVLSVAL | HLA-C03:04 | IAPP | Pancreas | 3176 |
| KATPIERCL | HLA-C03:04 | IAPP | Pancreas | 3177 |
| RQEWIIPVL | HLA-C04:01 | IAPP | Pancreas | 3178 |
| SSNNFGAIL | HLA-C04:01 | IAPP | Pancreas | 3179 |
| RWKSGNATL | HLA-C04:01 | IAPP | Pancreas | 3180 |
| HSSNNFGAI | HLA-C04:01 | IAPP | Pancreas | 3181 |
| FLIVLSVAL | HLA-C04:01 | IAPP | Pancreas | 3182 |
| CLDQIPIF | HLA-C04:01 | IAPP | Pancreas | 3183 |
| VLSVALNHL | HLA-C04:01 | IAPP | Pancreas | 3184 |
| ERQEWIIPV | HLA-C07:01 | IAPP | Pancreas | 3185 |
| NTYGKRNAV | HLA-C07:01 | IAPP | Pancreas | 3186 |
| LKREPLNYL | HLA-C07:01 | IAPP | Pancreas | 3187 |
| KRNAVEVL | HLA-C07:01 | IAPP | Pancreas | 3188 |
| SRNILLEL | HLA-C07:01 | IAPP | Pancreas | 3189 |
| RQEWHIPVL | HLA-C07:01 | IAPP | Pancreas | 3190 |
| ERQEWIIPV | HLA-C07:02 | IAPP | Pancreas | 3191 |
| LKREPLNYL | HLA-C07:02 | IAPP | Pancreas | 3192 |
| RWKSGNATL | HLA-C07:02 | IAPP | Pancreas | 3193 |
| KRNAVEVI | HLA-C07:02 | IAPP | Pancreas | 3194 |
| SRNILLEL | HLA-C07:02 | IAPP | Pancreas | 3195 |
| KREPLNYL | HLA-C07:02 | IAPP | Pancreas | 3196 |
| FGDIQEGIY | HLA-A01:01 | KIF2B | Testis | 3197 |
| VTEINRENY | HLA-A01:01 | KIF2B | Testis | 3198 |
| STYEKLDLKVY | HLA-A01:01 | KIF2B | Testis | 3199 |
| PSDNVVMVH | HLA-A01:01 | KIF2B | Testis | 3200 |
| TSCENTLNTLRY | HLA-A01:01 | KIF2B | Testis | 3201 |
| KIDLETILL | HLA-A01:01 | KIF2B | Testis | 3202 |
| YQFTAQPLV | HLA-A02:01 | KIF2B | Testis | 3203 |
| KVYGTFFEI | HLA-A02:01 | KIF2B | Testis | 3204 |
| KLLLADLHV | HLA-A02:01 | KIF2B | Testis | 3205 |
| YALVAQDVFL | HLA-A02:01 | KIF2B | Testis | 3206 |
| ALKECILAL | HLA-A02:01 | KIF2B | Testis | 3207 |
| IMHGKFSLV | HLA-A02:01 | KIF2B | Testis | 3208 |
| ALVAQDVFL | HLA-A02:01 | KIF2B | Testis | 3209 |
| ALVAQDVFLL | HLA-A02:01 | KIF2B | Testis | 3210 |
| RIMHGKFSLV | HLA-A02:01 | KIF2B | Testis | 3211 |
| LLLADLHV | HLA-A02:01 | KIF2B | Testis | 3212 |
| TLKDLDIITV | HLA-A02:01 | KIF2B | Testis | 3213 |
| HLDSSKISV | HLA-A02:01 | KIF2B | Testis | 3214 |
| ALTEIQKKL | HLA-A02:01 | KIF2B | Testis | 3215 |
| GLQEKEVCCV | HLA-A02:01 | KIF2B | Testis | 3216 |
| TLLGKDTTI | HLA-A02:01 | KIF2B | Testis | 3217 |
| VLEDGNQQI | HLA-A02:01 | KIF2B | Testis | 3218 |
| CLWEIQKL | HLA-A02:01 | KIF2B | Testis | 3219 |
| KVYDLLNWK | HLA-A03:01 | KIF2B | Testis | 3220 |
| LLLRNSTYEK | ILA-A03:01 | KIF2B | Testis | 3221 |
| KVYDLLNWKK | HLA-A03:01 | KIF2B | Testis | 3222 |
| RVPSKPCLMK | HLA-A03:01 | KIF2B | Testis | 3223 |
| KVYGTFFEIY | HLA-A03:01 | KIF2B | Testis | 3224 |
| STYEKLDLK | HLA-A03:01 | KIF2B | Testis | 3225 |
| STYEKLDLK | HLA-A11:01 | KIF2B | Testis | 3226 |
| KVYDLLNWK | HLA-A11:01 | KIF2B | Testis | 3227 |
| VETLPTLLGK | HLA-A11:01 | KIF2B | Testis | 3228 |
| KVYDLLNWKK | HLA-A11:01 | KIF2B | Testis | 3229 |
| GTFFEIYGGK | HLA-A11:01 | KIF2B | Testis | 3230 |
| AVFQIILK | HLA-A11:01 | KIF2B | Testis | 3231 |
| RQLEGAEINK | HLA-ALI:01 | KIF2B | Testis | 3232 |
| RYLQNQTFCF | HLA-A24:02 | KIF2B | Testis | 3233 |
| IYALVAQDVF | HLA-A24:02 | KIF2B | Testis | 3234 |
| IYGGKVYDLL | HLA-A24:02 | KIF2B | Testis | 3235 |
| VYQFTAQPL | HLA-A24:02 | KIF2B | Testis | 3236 |
| IYGGKVYDL | HLA-A24:02 | KIF2B | Testis | 3237 |
| RYANRVKKL | HLA-A24:02 | KIF2B | Testis | 3238 |
| RTSRQTPVNA | HLA-A30:01 | KIF2B | Testis | 3239 |
| RVKKLNVDV | HLA-A30:01 | KIF2B | Testis | 3240 |
| RSDKRIHLAV | HLA-A30:01 | KIF2B | Testis | 3241 |
| LLRNSTYEK | HLA-A30:01 | KIF2B | Testis | 3242 |
| KVYDLLNWK | HLA-A30:01 | KIF2B | Testis | 3243 |
| RSLSILEQK | HLA-A30:01 | KIF2B | Testis | 3244 |
| TFFEIYGGK | HLA-A30:01 | KIF2B | Testis | 3245 |
| KVYDLLNWKK | HLA-A30:01 | KIF2B | Testis | 3246 |
| IMHMIEEYR | HLA-A33:03 | KIF2B | Testis | 3247 |
| EIMHMIEEYR | HLA-A33:03 | KIF2B | Testis | 3248 |
| HYPIGHEAPR | HLA-A33:03 | KIF2B | Testis | 3249 |
| NVDVRPYHR | HLA-A33:03 | KIF2B | Testis | 3250 |
| QWLENIQER | HLA-A33:03 | KIF2B | Testis | 3251 |
| EGIYVAIQR | HLA-A33:03 | KIF2B | Testis | 3252 |
| EIQKLQEQR | HLA-A33:03 | KIF2B | Testis | 3253 |
| RPYHRGHYPI | HLA-B07:02 | KIF2B | Testis | 3254 |
| RPLNQRETTL | HLA-B07:02 | KIF2B | Testis | 3255 |
| HPMPPPPLSPL | HLA-B07:02 | KIF2B | Testis | 3256 |
| TPFRASKLIL | HLA-B07:02 | KIF2B | Testis | 3257 |
| MPPPPLSPL | HLA-B07:02 | KIF2B | Testis | 3258 |
| MPPPPLSPLAL | HLA-B07:02 | KIF2B | Testis | 3259 |
| HPMPPPPL | HLA-B07:02 | KIF2B | Testis | 3260 |
| LMKQKKSPCL | HLA-B08:01 | KIF2B | Testis | 3261 |
| LMKQKKSPC | HLA-B08:01 | KIF2B | Testis | 3262 |
| RPYHRGHYPI | HLA-B08:01 | KIF2B | Testis | 3263 |
| EIRARRAL | HLA-B08:01 | KIF2B | Testis | 3264 |
| NWKKKLQVL | HLA-B08:01 | KIF2B | Testis | 3265 |
| EIQKKLKLL | HLA-B08:01 | KIF2B | Testis | 3266 |
| ESKQKVDL | HLA-B08:01 | KIF2B | Testis | 3267 |
| ALKECILAL | HLA-B08:01 | KIF2B | Testis | 3268 |
| YQFTAQPLV | HLA-B13:02 | KIF2B | Testis | 3269 |
| KVYGTFFEL | HLA-B13:02 | KIF2B | Testis | 3270 |
| LDLKVYGTFFEI | HLA-B13:02 | KIF2B | Testis | 3271 |
| IDFCIARSLSI | HLA-B13:02 | KIF2B | Testis | 3272 |
| SLSILEQKI | HLA-B13:02 | KIF2B | Testis | 3273 |
| GQNSSTCMI | HLA-B13:02 | KIF2B | Testis | 3274 |
| LQRDEFIKI | HLA-B13:02 | KIF2B | Testis | 3275 |
| MVHESKQKV | HLA-B13:02 | KIF2B | Testis | 3276 |
| YALVAQDVF | HLA-B46:01 | KIF2B | Testis | 3277 |
| FLLLRNSTY | HLA-B46:01 | KIF2B | Testis | 3278 |
| QTFCFDHAF | HLA-B46:01 | KIF2B | Testis | 3279 |
| RASKLTLVL | HLA-B46:01 | KIF2B | Testis | 3280 |
| EIMHMIEEY | HLA-B46:01 | KIF2B | Testis | 3281 |
| IMHMIEEY | HLA-B46:01 | KIF2B | Testis | 3282 |
| YEIMHMIEEY | HLA-B46:01 | KIF2B | Testis | 3283 |
| HAFDDKASN | HLA-B46:01 | KIF2B | Testis | 3284 |
| MPPPPLSPL | HLA-C01:02 | KIF2B | Testis | 3285 |
| RVPSKPCLM | HLA-C01:02 | KIF2B | Testis | 3286 |
| RASKLTLVL | HLA-C01:02 | KIF2B | Testis | 3287 |
| VAQDVFLLL | HLA-C01:02 | KIF2B | Testis | 3288 |
| CLPESPCL | HLA-C01:02 | KIF2B | Testis | 3289 |
| PMPPPPLSPL | HLA-C01:02 | KIF2B | Testis | 3290 |
| ESPCLSPL | HLA-C01:02 | KIF2B | Testis | 3291 |
| LALAPSSAI | HLA-C03:04 | KIF2B | Testis | 3292 |
| RASKLTLVL | HLA-C03:04 | KIF2B | Testis | 3293 |
| YALVAQDVF | HLA-C03:04 | KIF2B | Testis | 3294 |
| VAQDVFLLL | HLA-C03:04 | KIF2B | Testis | 3295 |
| FCLPESPCL | HLA-C03:04 | KIF2B | Testis | 3296 |
| LALKECILAL | HLA-C03:04 | KIF2B | Testis | 3297 |
| AFDDKASNEL | HLA-C04:01 | KIF2B | Testis | 3298 |
| RVPSKPCLM | HLA-C04:01 | KIF2B | Testis | 3299 |
| YLQNQTFCF | HLA-C04:01 | KIF2B | Testis | 3300 |
| YQFTAQPLV | HLA-C04:01 | KIF2B | Testis | 3301 |
| MPPPPLSPL | HLA-C04:01 | KIF2B | Testis | 3302 |
| NYWVTVEWV | HLA-C04:01 | KIF2B | Testis | 3303 |
| FDDKASNEL | HLA-C04:01 | KIF2B | Testis | 3304 |
| KIDLETILL | HLA-C04:01 | KIF2B | Testis | 3305 |
| FRASKLTLV | HLA-C07:01 | KIF2B | Testis | 3306 |
| FRASKLTLVL | HLA-C07:01 | KIF2B | Testis | 3307 |
| FRKGMATCF | HLA-C07:01 | KIF2B | Testis | 3308 |
| TRYLQNQTP | HLA-C07:01 | KIF2B | Testis | 3309 |
| TRNPNYEIM | HLA-C07:01 | KIF2B | Testis | 3310 |
| ERAGGVHHD | HLA-C07:01 | KIF2B | Testis | 3311 |
| TRNPNYEI | HLA-C07:01 | KIF2B | Testis | 3312 |
| FRASKLTLV | HLA-C07:02 | KIF2B | Testis | 3313 |
| FRASKLTLVL | HLA-C07:02 | KIF2B | Testis | 3314 |
| FRKGMATCF | HLA-C07:02 | KIF2B | Testis | 3315 |
| RYANRVKKL | HLA-C07:02 | KIF2B | Testis | 3316 |
| TRNPNYEIM | HLA-C07:02 | KIF2B | Testis | 3317 |
| VRPYHRGHY | HLA-C07:02 | KIF2B | Testis | 3318 |
| VYGTFFETY | HLA-C07:02 | KIF2B | Testis | 3319 |
| ETKDPTNGYY | HLA-A01:01 | KIRREL2 | Pancreas | 3320 |
| TLETKDPTNGYY | HLA-A01:01 | KIRREL2 | Pancreas | 3321 |
| LETKDPTNGYY | HLA-A01:01 | KIRREL2 | Pancreas | 3322 |
| DTAITLSLQY | HLA-A01:01 | KIRREL2 | Pancreas | 3323 |
| TKDPTNGYY | HLA-A01:01 | KIRREL2 | Pancreas | 3324 |
| TLETKDPINGY | HLA-A01:01 | KIRREL2 | Pancreas | 3325 |
| FLTEPVSCEV | HLA-A02:01 | KIRREL2 | Pancreas | 3326 |
| GLGPQLISV | HLA-A02:01 | KIRREL2 | Pancreas | 3327 |
| FLVETFPA | HLA-A02:01 | KIRREL2 | Pancreas | 3328 |
| TLLMVITGV | HLA-A02:01 | KIRREL2 | Pancreas | 3329 |
| MLRMRVPALL | HLA-A02:01 | KIRREL2 | Pancreas | 3330 |
| VLFGPILQA | HLA-A02:01 | KIRREL2 | Pancreas | 3331 |
| FLCQATAQPPV | HLA-A02:01 | KIRREL2 | Pancreas | 3332 |
| ILQAKPEPV | HLA-A02:01 | KIRREL2 | Pancreas | 3333 |
| FLQQPEDLVVLL | HLA-A02:01 | KIRREL2 | Pancreas | 3334 |
| FLQQPEDLV | HLA-A02:01 | KIRREL2 | Pancreas | 3335 |
| TTLLMVITGV | HLA-A02:01 | KIRREL2 | Pancreas | 3336 |
| LLMVITGV | HLA-A02:01 | KIRREL2 | Pancreas | 3337 |
| TLSASPHTV | HLA-A02:01 | KIRREL2 | Pancreas | 3338 |
| VLLGEEARL | HLA-A02:01 | KIRREL2 | Pancreas | 3339 |
| VLGGPSVSL | HLA-A02:01 | KIRREL2 | Pancreas | 3340 |
| VLVPPEAPQV | HLA-A02:01 | KIRREL2 | Pancreas | 3341 |
| RLGEGGAQA | HLA-A02:01 | KIRREL2 | Pancreas | 3342 |
| ALHSAPAFL | HLA-A02:01 | KIRREL2 | Pancreas | 3343 |
| FLQQPEDLVV | HLA-A02:01 | KIRREL2 | Pancreas | 3344 |
| ATFHQTLLK | HLA-A03:01 | KIRREL2 | Pancreas | 3345 |
| VLFGPILQAK | HLA-A03:01 | KIRREL2 | Pancreas | 3346 |
| GATFHQTLLK | HLA-A03:01 | KIRREL2 | Pancreas | 3347 |
| RLYRARAGY | HLA-A03:01 | KIRREL2 | Pancreas | 3348 |
| VLFGPILQA | HLA-A03:01 | KIRREL2 | Pancreas | 3349 |
| ATAQPPVTGY | HLA-A03:01 | KIRREL2 | Pancreas | 3350 |
| ATFHQTLLK | HLA-A11:01 | KIRREL2 | Pancreas | 3351 |
| GATFHQTLLK | HLA-A11:01 | KIRREL2 | Pancreas | 3352 |
| ASASFSEQK | HLA-A11:01 | KIRREL2 | Pancreas | 3353 |
| VLFGPILQAK | HLA-A11:01 | KIRREL2 | Pancreas | 3354 |
| ATFHQTLLKE | HLA-A11:01 | KIRREL2 | Pancreas | 3355 |
| ATQAGLRSR | HLA-A11:01 | KIRREL2 | Pancreas | 3356 |
| GTQESDFSR | HLA-A11:01 | KIRREL2 | Pancreas | 3357 |
| RVPALLVLLF | HLA-A24:02 | KIRREL2 | Pancreas | 3358 |
| LWFRDGVLL | HLA-A24:02 | KIRREL2 | Pancreas | 3359 |
| SYIKPTSF | HLA-A24:02 | KIRREL2 | Pancreas | 3360 |
| RLPCALGAYW | HLA-A24:02 | KIRREL2 | Pancreas | 3361 |
| LQPPGTPTF | HLA-A24:02 | KIRREL2 | Pancreas | 3362 |
| QYPPEVTL | HLA-A24:02 | KIRREL2 | Pancreas | 3363 |
| PFSHDDGATF | HLA-A24:02 | KIRREL2 | Pancreas | 3364 |
| KVRQVSPPA | HLA-A30:01 | KIRREL2 | Pancreas | 3365 |
| RSRPAQLHV | HLA-A30:01 | KIRREL2 | Pancreas | 3366 |
| KVRGVSVSL | HLA-A30:01 | KIRREL2 | Pancreas | 6367 |
| RMRVPALLV | HLA-A30:01 | KIRREL2 | Pancreas | 3368 |
| ATFHQTLLK | HLA-A30:01 | KIRREL2 | Pancreas | 3369 |
| ETKDPTNGY | HLA-A30:01 | KIRREL2 | Pancreas | 3370 |
| MVPPCRLYR | HLA-A33:03 | KIRREL2 | Pancreas | 3371 |
| FSRSFNCSAR | HLA-A33:03 | KIRREL2 | Pancreas | 3372 |
| DASFSCAWR | HLA-A33:03 | KIRREL2 | Pancreas | 3373 |
| CFRGRAGWSR | HLA-A33:03 | KIRREL2 | Pancreas | 3374 |
| ETFPAPESR | HLA-A33:03 | KIRREL2 | Pancreas | 3375 |
| EVSNAVGSANR | HLA-A33:03 | KIRREL2 | Pancreas | 3376 |
| SFNCSARNR | HLA-A33:03 | KIRREL2 | Pancreas | 3377 |
| EDAGDYVCR | HLA-A33:03 | KIRREL2 | Pancreas | 3378 |
| APAFLRGPA | HLA-B07:02 | KIRREL2 | Pancreas | 3379 |
| HPRAFTSYI | HLA-B07:02 | KIRREL2 | Pancreas | 3380 |
| FPAPESRGGL | HLA-B07:02 | KIRREL2 | Pancreas | 3381 |
| APAFLRGPARL | HLA-B07:02 | KIRREL2 | Pancreas | 3382 |
| SPLGPPGTPTF | HLA-B07:02 | KIRREL2 | Pancreas | 3383 |
| SPDSRVTSF | HLA-B07:02 | KIRREL2 | Pancreas | 3384 |
| APQVLGGPSV | HLA-B07:02 | KIRREL2 | Pancreas | 3385 |
| VPPEAPQVL | HLA-B07:02 | KIRREL2 | Pancreas | 3386 |
| MLRMRVPAL | HLA-B08:01 | KIRREL2 | Pancreas | 3387 |
| MLRMRVPALL | HLA-B08:01 | KIRREL2 | Pancreas | 3388 |
| MLRMRVPALLVL | HLA-B08:01 | KIRREL2 | Pancreas | 3389 |
| FVCRARSQAL | HLA-B08:01 | KIRREL2 | Pancreas | 3390 |
| DLHIRPVEL | HLA-B08:01 | KIRREL2 | Pancreas | 3391 |
| YYKVRGVSV | HLA-B08:01 | KIRREL2 | Pancreas | 3392 |
| TSYIKPTSF | HLA-B08:01 | KIRREL2 | Pancreas | 3393 |
| YDFNPHLGMV | HLA-B13:02 | KIRREL2 | Pancreas | 3394 |
| RDLLPTVRI | HLA-B13:02 | KIRREL2 | Pancreas | 3395 |
| ADASFLTEPV | HLA-B13:02 | KIRREL2 | Pancreas | 3396 |
| RDLLPTVRIV | HLA-B13:02 | KIRREL2 | Pancreas | 3397 |
| CQATAQPPV | HLA-B13:02 | KIRREL2 | Pancreas | 3398 |
| GQHDLHIRP | HLA-B13:02 | KIRREL2 | Pancreas | 3399 |
| GQHDLHIRPV | HLA-B13:02 | KIRREL2 | Pancreas | 3400 |
| GLISVLHI | HLA-B13:02 | KIRREL2 | Pancreas | 3401 |
| LQYPPEVTL | HLA-B13:02 | KIRREL2 | Pancreas | 3402 |
| FSHDDGATF | HLA-B46:01 | KIRREL2 | Pancreas | 3403 |
| FASPAPDAV | HLA-B46:01 | KIRREL2 | Pancreas | 3404 |
| FTSYIKPTSF | HLA-B46:01 | KIRREL2 | Pancreas | 3405 |
| AAATTTLLM | HLA-B46:01 | KIRREL2 | Pancreas | 3406 |
| TSYIKPTSF | HLA-B46:01 | KIRREL2 | Pancreas | 3407 |
| TAQPPVTGY | HLA-B46:01 | KIRREL2 | Pancreas | 3408 |
| YAAFPTPSH | HLA-B46:01 | KIRREL2 | Pancreas | 3409 |
| VAAATTTLL | HLA-C01:02 | KIRREL2 | Pancreas | 3410 |
| FLPPPSPL | HLA-C01:02 | KIRREL2 | Pancreas | 3411 |
| RVPALLVLL | HLA-C01:02 | KIRREL2 | Pancreas | 3412 |
| NAPPVVTAL | HLA-C01:02 | KIRREL2 | Pancreas | 3413 |
| EAPGQGLFL | HLA-C01:02 | KIRREL2 | Pancreas | 3414 |
| RGPARLQCL | HLA-C01:02 | KIRREL2 | Pancreas | 3415 |
| VAAATTTLL | HLA-C03:04 | KIRREL2 | Pancreas | 3416 |
| FASPAPDAV | HLA-C03:04 | KIRREL2 | Pancreas | 3417 |
| FSHDDGATF | HLA-C03:04 | KIRREL2 | Pancreas | 3418 |
| AAATTTLLM | HLA-C03:04 | KIRREL2 | Pancreas | 3419 |
| NAPPVVTAL | HLA-C03:04 | KIRREL2 | Pancreas | 3420 |
| LQYPPEVTL | HLA-C03:04 | KIRREL2 | Pancreas | 3421 |
| YAAFPTPSH | HLA-C03:04 | KIRREL2 | Pancreas | 3422 |
| FYDFNPHLGM | HLA-C04:01 | KIRREL2 | Pancreas | 3423 |
| SHDDGATFV | HLA-C04:01 | KIRREL2 | Pancreas | 3424 |
| FYDFNPHL | HLA-C04:01 | KIRREL2 | Pancreas | 3425 |
| FYDFNPHLG | HLA-C04:01 | KIRREL2 | Pancreas | 3426 |
| QYPPEVTL | HLA-C04:01 | KIRREL2 | Pancreas | 3427 |
| LRMRVPALL | HLA-C07:01 | KIRREL2 | Pancreas | 3428 |
| MRVPALLVL | HLA-C07:01 | KIRREL2 | Pancreas | 3429 |
| MLRMRVPALL | HLA-C07:01 | KIRREL2 | Pancreas | 3430 |
| TRRGGAQVL | HLA-C07:01 | KIRREL2 | Pancreas | 3431 |
| RRGGAQVL | HLA-C07:01 | KIRREL2 | Pancreas | 3432 |
| DRGPIVHTD | HLA-C07:01 | KIRREL2 | Pancreas | 3433 |
| SRPAQLHVL | HLA-C07:01 | KIRREL2 | Pancreas | 3434 |
| LRMRVPALL | HLA-C07:02 | KIRREL2 | Pancreas | 3435 |
| MRVPALLVL | HLA-C07:02 | KIRREL2 | Pancreas | 3436 |
| SRPAQLHVL | HLA-C07:02 | KIRREL2 | Pancreas | 3437 |
| LYRARAGYL | HLA-C07:02 | KIRREL2 | Pancreas | 3438 |
| QYPPEVTL | HLA-C07:02 | KIRREL2 | Pancreas | 3439 |
| NAPPVVTAL | HLA-C07:02 | KIRREL2 | Pancreas | 3440 |
| LSNDMCARAY | HLA-A01:01 | KLK2 | Prostate | 3441 |
| HSQPWQVAVY | HLA-A01:01 | KLK2 | Prostate | 3442 |
| YSEKVTEFM | HLA-A01:01 | KLK2 | Prostate | 3443 |
| STCLLGTCY | HLA-A01:01 | KLK2 | Prostate | 3444 |
| LSTCLLGTCY | HLA-A01:01 | KLK2 | Prostate | 3445 |
| DTCGVSHPY | HLA-A01:01 | KLK2 | Prostate | 3446 |
| LLSNDMCARAY | HLA-A01:01 | KLK2 | Prostate | 3447 |
| HLLSNDMCARAY | HLA-A01:01 | KLK2 | Prostate | 3448 |
| SNDMCARAY | HLA-A01:01 | KLK2 | Prostate | 3449 |
| ITDVVKVLG | HLA-A01:01 | KLK2 | Prostate | 3450 |
| YSEKVTEF | HLA-A01:01 | KLK2 | Prostate | 3451 |
| ITDVVKVLGL | HLA-A01:01 | KLK2 | Prostate | 3452 |
| YSEKVTEFMLC | HLA-A01:01 | KLK2 | Prostate | 3453 |
| YSEKVTEFML | HLA-A01:01 | KLK2 | Prostate | 3454 |
| ITDVVKVL | HLA-A01:01 | KLK2 | Prostate | 3455 |
| SLQCVSLHL | HLA-A02:01 | KLK2 | Prostate | 3456 |
| LVLSIALSV | HLA-A02:01 | KLK2 | Prostate | 3457 |
| VILGVHLSV | HLA-A02:01 | KLK2 | Prostate | 3458 |
| VLAPQESSV | HLA-A02:01 | KLK2 | Prostate | 3459 |
| FMLCAGLWTG | HLA-A02:01 | KLK2 | Prostate | 3460 |
| ILGVHLSVMV | HLA-A02:01 | KLK2 | Prostate | 3461 |
| SLLCTPRWCI | HLA-A02:01 | KLK2 | Prostate | 3462 |
| FMLCAGLWT | HLA-A02:01 | KLK2 | Prostate | 3463 |
| SLQCVSLHLL | HLA-A02:01 | KLK2 | Prostate | 3464 |
| VLSTCLLGTCYV | HLA-A02:01 | KLK2 | Prostate | 3465 |
| HLSVMVCFKV | HLA-A02:01 | KLK2 | Prostate | 3466 |
| LLGTCYVTSI | HLA-A02:01 | KLK2 | Prostate | 3467 |
| GLWTGGKDTCGV | HLA-A02:01 | KLK2 | Prostate | 3468 |
| MWDLVLSI | HLA-A02:01 | KLK2 | Prostate | 3469 |
| FLRPRSLQCV | HLA-A02:01 | KLK2 | Prostate | 3470 |
| RVILGVHLSV | HLA-A02:01 | KLK2 | Prostate | 3471 |
| ALSVGCTGI | HLA-A02:01 | KLK2 | Prostate | 3472 |
| RSLQCVSLHL | HLA-A02:01 | KLK2 | Prostate | 3473 |
| ALSVQCTGAV | HLA-A02:01 | KLK2 | Prostate | 3474 |
| CLLGTCYV | HLA-A02:01 | KLK2 | Prostate | 3475 |
| VLSTCLLGT | HLA-A02:01 | KLK2 | Prostate | 3476 |
| LLSNDMCARA | HLA-A02:01 | KLK2 | Prostate | 3477 |
| WLRVILGVHLSV | HLA-A02:01 | KLK2 | Prostate | 3478 |
| KVTEFMLCA | HLA-A02:01 | KLK2 | Prostate | 3479 |
| CLLGTCYVT | HLA-A02:01 | KLK2 | Prostate | 3480 |
| SVCEPPSPV | HLA-A02:01 | KLK2 | Prostate | 3481 |
| MLCAGLWTG | HLA-A02:01 | KLK2 | Prostate | 3482 |
| DLVLSIALSV | HLA-A02:01 | KLK2 | Prostate | 3483 |
| LLGTCYVTSIAV | HLA-A02:01 | KLK2 | Prostate | 3484 |
| GLPTQEPAL | HLA-A02:01 | KLK2 | Prostate | 3485 |
| ALPEKPAV | HLA-A02:01 | KLK2 | Prostate | 3486 |
| RLSEPAKITDV | HLA-A02:01 | KLK2 | Prostate | 3487 |
| AKITDVVKV | HLA-A02:01 | KLK2 | Prostate | 3488 |
| ALPEKPAVYT | HLA-A02:01 | KLK2 | Prostate | 3489 |
| KITDVVKV | HLA-A02:01 | KLK2 | Prostate | 3490 |
| KITDVVKVL | HLA-A02:01 | KLK2 | Prostate | 3491 |
| ALPEKPAVY | HLA-A02:01 | KLK2 | Prostate | 3492 |
| AWLRVILGV | HLA-A02:01 | KLK2 | Prostate | 3493 |
| STGDHLLRL | HLA-A02:01 | KLK2 | Prostate | 3494 |
| HLLSNDMCA | HLA-A02:01 | KLK2 | Prostate | 3495 |
| SLLKHQSL | HLA-A02:01 | KLK2 | Prostate | 3496 |
| ALSVGCTGA | HLA-A02:01 | KLK2 | Prostate | 3497 |
| RLSEPAKI | HLA-A02:01 | KLK2 | Prostate | 3498 |
| ALSHVPCL | HLA-A02:01 | KLK2 | Prostate | 3499 |
| FLRPRSL?C | HLA-A02:01 | KLK2 | Prostate | 3500 |
| NLFEPEDTG | HLA-A02:01 | KLK2 | Prostate | 3501 |
| HPLYNMSLLK | HLA-A03:01 | KLK2 | Prostate | 3502 |
| ALSHVPCLK | HLA-A03:01 | KLK2 | Prostate | 3503 |
| PLYNMSLLK | HLA-A03:01 | KLK2 | Prostate | 3504 |
| FPHPLYNMSLLK | HLA-A03:01 | KLK2 | Prostate | 3505 |
| HLSVMVCFK | HLA-A03:01 | KLK2 | Prostate | 3506 |
| PHPLYNMSLLK | HLA-A03:01 | KLK2 | Prostate | 3507 |
| VHLSVMVCFK | HLA-A03:01 | KLK2 | Prostate | 3508 |
| MLLRLSEPAK | HLA-A03:01 | KLK2 | Prostate | 3509 |
| GALSHVPCLK | HLA-A03:01 | KLK2 | Prostate | 3510 |
| AVYTKVVHY | HLA-A03:01 | KLK2 | Prostate | 3511 |
| AVYSHGWAH | HLA-A03:01 | KLK2 | Prostate | 3512 |
| AVYTKVVHYR | HLA-A03:01 | KLK2 | Prostate | 3513 |
| ALPEKPAVY | HLA-A03:01 | KLK2 | Prostate | 3514 |
| AVYTKVVHYRK | HLA-A03:01 | KLK2 | Prostate | 3515 |
| AVDGAGQKK | HLA-A03:01 | KLK2 | Prostate | 3516 |
| HPLYNMSLLK | HLA-A11:01 | KLK2 | Prostate | 3517 |
| AVYTKVVHYR | HLA-A11:01 | KLK2 | Prostate | 3518 |
| ALSHVPCLK | HLA-A11:01 | KLK2 | Prostate | 3519 |
| HLSVMVCFK | HLA-A11:01 | KLK2 | Prostate | 3520 |
| GALSHVPCLK | HLA-A11:01 | KLK2 | Prostate | 3521 |
| PLYNMSLLK | HLA-A11:01 | KLK2 | Prostate | 3522 |
| AVYTKVVHY | HLA-A11:01 | KLK2 | Prostate | 3523 |
| VHLSVMVCFK | HLA-A11:01 | KLK2 | Prostate | 3524 |
| VTSIAVDPGK | HLA-A11:01 | KLK2 | Prostate | 3525 |
| AVYTKVVHYRK | HLA-A11:01 | KLK2 | Prostate | 3526 |
| AVDGAGQKK | HLA-A11:01 | KLK2 | Prostate | 3527 |
| ASTGDHLLR | HLA-A11:01 | KLK2 | Prostate | 3528 |
| SVGCTGIAR | HLA-A11:01 | KLK2 | Prostate | 3529 |
| GSIEPEEFLR | HLA-A11:01 | KLK2 | Prostate | 3530 |
| AVYSHGWAH | HLA-A11:01 | KLK2 | Prostate | 3531 |
| SIEPEEFLR | HLA-ALI:01 | KLK2 | Prostate | 3532 |
| VWLGRHNLF | HLA-A24:02 | KLK2 | Prostate | 3533 |
| AYSEKVTEF | HLA-A24:02 | KLK2 | Prostate | 3534 |
| RAYSEKVTEF | HLA-A24:02 | KLK2 | Prostate | 3535 |
| QVWLGRHNLF | HLA-A24:02 | KLK2 | Prostate | 3536 |
| SFPHPLYNM | HLA-A24:02 | KLK2 | Prostate | 3537 |
| EFMLCAGLW | HLA-A24:02 | KLK2 | Prostate | 3538 |
| TEFMLCAGLW | HLA-A24:02 | KLK2 | Prostate | 3539 |
| VYTKVVHYRKWI | HLA-A24:02 | KLK2 | Prostate | 3540 |
| VYTKVVHYRKW | HLA-A24:02 | KLK2 | Prostate | 3541 |
| VYTKVVHYR | HLA-A24:02 | KLK2 | Prostate | 3542 |
| VYSHGWAHC | HLA-A24:02 | KLK2 | Prostate | 3543 |
| QRVPVSHSF | HLA-A24:02 | KLK2 | Prostate | 3544 |
| SWGPEPCAL | HLA-A24:02 | KLK2 | Prostate | 3545 |
| VHLSVMVCF | HLA-A24:02 | KLK2 | Prostate | 3546 |
| RTRQVLAPQ | HLA-A30:01 | KLK2 | Prostate | 3547 |
| KTQARGSLS | HLA-A30:01 | KLK2 | Prostate | 3548 |
| KTQARGSLSA | HLA-A30:01 | KLK2 | Prostate | 3549 |
| RTRGVLAPQE | HLA-A30:01 | KLK2 | Prostate | 3550 |
| RTRGVLAPQESS | HLA-A30:01 | KLK2 | Prostate | 3551 |
| RTRGVLAPQES | HLA-A30:01 | KLK2 | Prostate | 3552 |
| RTRGVLAP | HLA-A30:01 | KLK2 | Prostate | 3553 |
| LLRLSEPAK | HLA-A30:01 | KLK2 | Prostate | 3554 |
| GSRTPSQPT | HLA-A30:01 | KLK2 | Prostate | 3555 |
| AVYTKVVHY | HLA-A30:01 | KLK2 | Prostate | 3556 |
| KITDVVKVL | HLA-A30:01 | KLK2 | Prostate | 3557 |
| AVDGAGQKK | HLA-A30:01 | KLK2 | Prostate | 3558 |
| LVLSIALSV | HLA-A30:01 | KLK2 | Prostate | 3559 |
| SVCEPPSPV | HLA-A30:01 | KLK2 | Prostate | 3560 |
| RVILGVHLSV | HLA-A30:01 | KLK2 | Prostate | 3561 |
| VILGVHLSV | HLA-A30:01 | KLK2 | Prostate | 3562 |
| TSIAVDPGK | HLA-A30:01 | KLK2 | Prostate | 3563 |
| VYTKVVHYR | HLA-A33:03 | KLK2 | Prostate | 3564 |
| MSLLKHQSLR | HLA-A33:03 | KLK2 | Prostate | 3565 |
| AVYTKVVHYR | HLA-A33:03 | KLK2 | Prostate | 3566 |
| CLKSLLCTPR | HLA-A33:03 | KLK2 | Prostate | 3567 |
| SLLKHQSLR | HLA-A33:03 | KLK2 | Prostate | 3568 |
| LSATASHTR | HLA-A33:03 | KLK2 | Prostate | 3569 |
| INMSLLKHQSLR | HLA-A33:03 | KLK2 | Prostate | 3570 |
| SVGCTGIAR | HLA-A33:03 | KLK2 | Prostate | 3571 |
| YNMSLLKHQSLR | HLA-A33:03 | KLK2 | Prostate | 3572 |
| EPEEFLRPR | HLA-A33:03 | KLK2 | Prostate | 3573 |
| GAVPLIQSR | HLA-A33:03 | KLK2 | Prostate | 3574 |
| STGDHLLRLR | HLA-A33:03 | KLK2 | Prostate | 3575 |
| SIEPEEFLR | HLA-A33:03 | KLK2 | Prostate | 3576 |
| TGDHLLRLR | HLA-A33:03 | KLK2 | Prostate | 3577 |
| RPRSLQCVSL | HLA-B07:02 | KLK2 | Prostate | 3578 |
| RPRSLQCVSLHL | HLA-B07:02 | KLK2 | Prostate | 3579 |
| FPHPLYNMSI | HLA-B07:02 | KLK2 | Prostate | 3580 |
| KPAVYTKVV | HLA-B07:02 | KLK2 | Prostate | 3581 |
| SPIPVLSTCL | HLA-B07:02 | KLK2 | Prostate | 3582 |
| HPLYNMSLL | HLA-B07:02 | KLK2 | Prostate | 3583 |
| HPQWVLTAAHCL | HLA-B07:02 | KLK2 | Prostate | 3584 |
| LRPRSLQCVSL | HLA-B07:02 | KLK2 | Prostate | 3585 |
| RPRSLQCVS | HLA-B07:02 | KLK2 | Prostate | 3586 |
| SPIPVLSTC | HLA-B07:02 | KLK2 | Prostate | 3587 |
| IPVLSTCLL | HLA-B07:02 | KLK2 | Prostate | 3588 |
| SPIPVLSTCLL | HLA-B07:02 | KLK2 | Prostate | 3589 |
| GPAHPGAST | HLA-B07:02 | KLK2 | Prostate | 3590 |
| TPSQPTPEC | HLA-B07:02 | KLK2 | Prostate | 3591 |
| FLRPRSLQCVSL | HLA-B08:01 | KLK2 | Prostate | 3592 |
| MSLLKHQSL | HLA-B08:01 | KLK2 | Prostate | 3593 |
| NMSLLKHQSL | HLA-B08:01 | KLK2 | Prostate | 3594 |
| FLRPRSLQCV | HLA-B08:01 | KLK2 | Prostate | 3595 |
| FLRPRSLQC | HLA-B08:01 | KLK2 | Prostate | 3596 |
| SLLKHQSL | HLA-B08:01 | KLK2 | Prostate | 3597 |
| FPHPLYNMSL | HLA-B08:01 | KLK2 | Prostate | 3598 |
| ILGVHLSVM | HLA-B08:01 | KLK2 | Prostate | 3599 |
| YNMSLLKHQSL | HLA-B08:01 | KLK2 | Prostate | 3600 |
| CFKVSHHGAL | HLA-B08:01 | KLK2 | Prostate | 3601 |
| EEFLRPRSL | HLA-B08:01 | KLK2 | Prostate | 3602 |
| DLVLSIAL | HLA-B08:01 | KLK2 | Prostate | 3603 |
| HPLYNMSL | HLA-B08:01 | KLK2 | Prostate | 3604 |
| VPCLKSLL | HLA-B08:01 | KLK2 | Prostate | 3605 |
| EPAKITDV | HLA-B08:01 | KLK2 | Prostate | 3606 |
| DVVKVLGL | HLA-B08:01 | KLK2 | Prostate | 3607 |
| HVPCLKSL | HLA-B08:01 | KLK2 | Prostate | 3608 |
| RAWLRVILGV | HLA-B13:02 | KLK2 | Prostate | 3609 |
| TEFMLCAGL | HLA-B13:02 | KLK2 | Prostate | 3610 |
| MWDLVLSIAL | HLA-B13:02 | KLK2 | Prostate | 3611 |
| TEFMLCAGLW | HLA-B13:02 | KLK2 | Prostate | 3612 |
| WDLVLSIAL | HLA-B13:02 | KLK2 | Prostate | 3613 |
| KDRAWLRVI | HLA-B13:02 | KLK2 | Prostate | 3614 |
| WAHCGGVLV | HLA-B13:02 | KLK2 | Prostate | 3615 |
| WDLVLSIALSV | HLA-B13:02 | KLK2 | Prostate | 3616 |
| LSVMVCFKV | HLA-B13:02 | KLK2 | Prostate | 3617 |
| AWLRVILGV | HLA-B13:02 | KLK2 | Prostate | 3618 |
| ALSVGCTGI | HLA-B13:02 | KLK2 | Prostate | 3619 |
| KITDVVKV | HLA-B13:02 | KLK2 | Prostate | 3620 |
| RLSEPAKI | HLA-B13:02 | KLK2 | Prostate | 3621 |
| GQHRTRGV | HLA-B13:02 | KLK2 | Prostate | 3622 |
| AKITDVVKV | HLA-B13:02 | KLK2 | Prostate | 3623 |
| VILGVHLSV | HLA-B13:02 | KLK2 | Prostate | 3624 |
| HSQPWQVAV | HLA-B13:02 | KLK2 | Prostate | 3625 |
| RAYSEKVTE | HLA-B13:02 | KLK2 | Prostate | 3626 |
| HSFPHPLYNM | HLA-B46:01 | KLK2 | Prostate | 3627 |
| HSQPWQVAVY | HLA-B46:01 | KLK2 | Prostate | 3628 |
| LSNDMCARAY | HLA-B46:01 | KLK2 | Prostate | 3629 |
| LSVGCTGAV | HLA-B46:01 | KLK2 | Prostate | 3630 |
| SQPWQVAVY | HLA-B46:01 | KLK2 | Prostate | 3631 |
| RAYSEKVTEF | HLA-B46:01 | KLK2 | Prostate | 3632 |
| WAHCGGVLV | HLA-B46:01 | KLK2 | Prostate | 3633 |
| YSEKVTEFM | HLA-B46:01 | KLK2 | Prostate | 3634 |
| LSTCLLGTCY | HLA-B46:01 | KLK2 | Prostate | 3635 |
| AVYTKVVHY | HLA-B46:01 | KLK2 | Prostate | 3636 |
| AVYSHGWAH | HLA-B46:01 | KLK2 | Prostate | 3637 |
| VSHPYSQHL | HLA-B46:01 | KLK2 | Prostate | 3638 |
| VSHHGALSH | HLA-B46:01 | KLK2 | Prostate | 3639 |
| ALPEKPAVY | HLA-B46:01 | KLK2 | Prostate | 3640 |
| VSHPYSQH | HLA-B46:01 | KLK2 | Prostate | 3641 |
| IARSGWVGT | HLA-B46:01 | KLK2 | Prostate | 3642 |
| HSQPWQVAV | HLA-B46:01 | KLK2 | Prostate | 3643 |
| MSLLKHQSL | HLA-C01:02 | KLK2 | Prostate | 3644 |
| VSHPYSQHL | HLA-C01:02 | KLK2 | Prostate | 3645 |
| FPHPLYNMSL | HLA-C01:02 | KLK2 | Prostate | 3646 |
| HVPCLKSLL | HLA-C01:02 | KLK2 | Prostate | 3647 |
| HSQPWQVAV | HLA-C01:02 | KLK2 | Prostate | 3648 |
| WVGTTCLSL | HLA-C01:02 | KLK2 | Prostate | 3649 |
| YSEKVTEFM | HLA-C01:02 | KLK2 | Prostate | 3650 |
| STGDHLLRL | HLA-C01:02 | KLK2 | Prostate | 3651 |
| ILGVHLSVM | HLA-C01:02 | KLK2 | Prostate | 3652 |
| WGPEPCAL | HLA-C01:02 | KLK2 | Prostate | 3653 |
| GLPTQEPAL | HLA-C01:02 | KLK2 | Prostate | 3654 |
| HVPCLKSL | HLA-C01:02 | KLK2 | Prostate | 3655 |
| ALPEKPAV | HLA-C01:02 | KLK2 | Prostate | 3656 |
| LAPQESSV | HLA-C01:02 | KLK2 | Prostate | 3657 |
| NSPIPVLST | HLA-C01:02 | KLK2 | Prostate | 3658 |
| ALPEKPAVY | HLA-C01:02 | KLK2 | Prostate | 3659 |
| PIPVLSTCL | HLA-C01:02 | KLK2 | Prostate | 3660 |
| MSLLKHQSL | HLA-C03:04 | KLK2 | Prostate | 3661 |
| LSVGCTGAV | HLA-C03:04 | KLK2 | Prostate | 3662 |
| WAHCGGVLV | HLA-C03:04 | KLK2 | Prostate | 3663 |
| VSHPYSQHL | HLA-C03:04 | KLK2 | Prostate | 3664 |
| HSQPWQVAV | HLA-C03:04 | KLK2 | Prostate | 3665 |
| TASHTRSTI | HLA-C03:04 | KLK2 | Prostate | 3666 |
| HSFPHPLYNM | HLA-C03:04 | KLK2 | Prostate | 3667 |
| FKVSHHGAL | HLA-C03:04 | KLK2 | Prostate | 3668 |
| GALSHVPCL | HLA-C03:04 | KLK2 | Prostate | 3669 |
| LAVDPGKQQ | HLA-C03:04 | KLK2 | Prostate | 3670 |
| KITDVVKVL | HLA-C03:04 | KLK2 | Prostate | 3671 |
| WAHCQGVL | HLA-C03:04 | KLK2 | Prostate | 3672 |
| LSHVPCLKSL | HLA-C03:04 | KLK2 | Prostate | 3673 |
| STGDHLLRL | HLA-C03:04 | KLK2 | Prostate | 3674 |
| SFPHPLYNM | HLA-C04:01 | KLK2 | Prostate | 3675 |
| YSEKVTEFM | HLA-C04:01 | KLK2 | Prostate | 3676 |
| MWDLVLSIAL | HLA-C04:01 | KLK2 | Prostate | 3677 |
| AYSEKVTEF | HLA-C04:01 | KLK2 | Prostate | 3678 |
| GHNSPIPVL | HLA-C04:01 | KLK2 | Prostate | 3679 |
| FPHPLYNMSL | HLA-C04:01 | KLK2 | Prostate | 3680 |
| SLQCVSLHL | HLA-C04:01 | KLK2 | Prostate | 3681 |
| HSQPWQVAV | HLA-C04:01 | KLK2 | Prostate | 3682 |
| HSFPHPLYNM | HLA-C04:01 | KLK2 | Prostate | 3683 |
| MWDLVLSI | HLA-C04:01 | KLK2 | Prostate | 3684 |
| ITDVVKVL | HLA-C04:01 | KLK2 | Prostate | 3685 |
| MWDLVLSIA | HLA-C04:01 | KLK2 | Prostate | 3686 |
| ITDVVKVLG | HLA-C04:01 | KLK2 | Prostate | 3687 |
| TGDHLLRL | HLA-C04:01 | KLK2 | Prostate | 3688 |
| SWGPEPCAL | HLA-C04:01 | KLK2 | Prostate | 3689 |
| HSFPHPLYNM | HLA-C07:01 | KLK2 | Prostate | 3690 |
| DRAWLRVIL | HLA-C07:01 | KLK2 | Prostate | 3691 |
| QRVPVSHSF | HLA-C07:01 | KLK2 | Prostate | 3692 |
| LRLSEPAKI | HLA-C07:01 | KLK2 | Prostate | 3693 |
| HSFPHPLYNMSL | HLA-C07:01 | KLK2 | Prostate | 3694 |
| HSQPWQVAV | HLA-C07:01 | KLK2 | Prostate | 3695 |
| VSHPYSQHL | HLA-C07:01 | KLK2 | Prostate | 3696 |
| KDRAWLRVIL | HLA-C07:01 | KLK2 | Prostate | 3697 |
| SHVPCLKSL | HLA-C07:01 | KLK2 | Prostate | 3698 |
| HNSPIPVL | HLA-C07:01 | KLK2 | Prostate | 3699 |
| HSQPWQVAVY | HLA-C07:01 | KLK2 | Prostate | 3700 |
| HSFPHPLYN | HLA-C07:01 | KLK2 | Prostate | 3701 |
| SWGPEPCAL | HLA-C07:01 | KLK2 | Prostate | 3702 |
| RSGWVGTTC | HLA-C07:01 | KLK2 | Prostate | 3703 |
| KITDVVKVL | HLA-C07:01 | KLK2 | Prostate | 3704 |
| ARSGWVGTT | HLA-C07:01 | KLK2 | Prostate | 3705 |
| AYSEKVTEF | HLA-C07:02 | KLK2 | Prostate | 3706 |
| HSFPHPLYNM | HLA-C07:02 | KLK2 | Prostate | 3707 |
| SFPHPLYNM | HLA-C07:02 | KLK2 | Prostate | 3708 |
| QRVPVSHSF | HLA-C07:02 | KLK2 | Prostate | 3709 |
| SHVPCLKSI | HLA-C07:02 | KLK2 | Prostate | 3710 |
| VWLGRHNLF | HLA-C07:02 | KLK2 | Prostate | 3711 |
| FPHPLYNMSL | HLA-C07:02 | KLK2 | Prostate | 3712 |
| LRPRSLQCV | HLA-C07:02 | KLK2 | Prostate | 3713 |
| VSHPYSQHL | HLA-C07:02 | KLK2 | Prostate | 3714 |
| FKVSHHGAL | HLA-C07:02 | KLK2 | Prostate | 3715 |
| VYTKVVHY | HLA-C07:02 | KLK2 | Prostate | 3716 |
| ALPEKPAVY | HLA-C07:02 | KLK2 | Prostate | 3717 |
| LRVILGVHL | HLA-C07:02 | KLK2 | Prostate | 3718 |
| LRPDEDSSH | HLA-C07:02 | KLK2 | Prostate | 3719 |
| KITDVVKVL | HLA-C07:02 | KLK2 | Prostate | 3720 |
| HNSPIPVL | HLA-C07:02 | KLK2 | Prostate | 3721 |
| STCSVSHPY | HLA-A01:01 | KLK3 | Prostate | 3722 |
| VSHSFPHPLY | HLA-A01:01 | KLK2; KLK3 | Prostate | 3723 |
| KSTCSVSHPY | HLA-A01:01 | KLK3 | Prostate | 3724 |
| IAQPAPCSQLLY | HLA-A01:01 | KLK3 | Prostate | 3725 |
| LTDAVKVMDL | HLA-A01:01 | KLK3 | Prostate | 3726 |
| CALPERPSLY | HLA-A01:01 | KLK3 | Prostate | 3727 |
| CSGDSGGPLV | HLA-A01:01 | KLK3 | Prostate | 3728 |
| SSHDLMLLRL | HLA-A01:01 | KLK2; KLK3 | Prostate | 3729 |
| QPAPCSQLLY | HLA-A01:01 | KLK3 | Prostate | 3730 |
| LSEPAELT | HLA-A01:01 | KLK3 | Prostate | 3731 |
| ALPERPSLY | HLA-A01:01 | KLK3 | Prostate | 3732 |
| LPERPSLY | HLA-A01:01 | KLK3 | Prostate | 3733 |
| LSEPAELTD | HLA-A01:01 | KLK3 | Prostate | 3734 |
| LTDAVKVMD | HLA-A01:01 | KLK3 | Prostate | 3735 |
| HSFPHPLY | HLA-A01:01 | KLK2; KLK3 | Prostate | 3736 |
| LTDAVKVM | HLA-A01:01 | KLK3 | Prostate | 3737 |
| LTMPALPMV | HLA-A02:01 | KLK3 | Prostate | 3738 |
| FLTLSVTWIA | HLA-A02:01 | KLK3 | Prostate | 3739 |
| KLQCVDLHV | HLA-A02:01 | KLK3 | Prostate | 3740 |
| ILITELTMPA | HLA-A02:01 | KLK3 | Prostate | 3741 |
| FLTLSVTWI | HLA-A02:01 | KLK3 | Prostate | 3742 |
| FLTLAVCGGV | HLA-A02:01 | KLK3 | Prostate | 3743 |
| MLLRLSEPA | HLA-A02:01 | KLK2; KLK3 | Prostate | 3744 |
| ELTMPALPMV | HLA-A02:01 | KLK3 | Prostate | 3745 |
| FLSLPAPLQA | HLA-A02:01 | KLK3 | Prostate | 3746 |
| WIAPPLQVLV | HLA-A02:01 | KLK3 | Prostate | 3747 |
| KKLQCVDLHV | HLA-A02:01 | KLK3 | Prostate | 3748 |
| FLTPKKLQCV | HLA-A02:01 | KLK3 | Prostate | 3749 |
| LITELTMPA | HLA-A02:01 | KLK3 | Prostate | 3750 |
| FLSLPAPL | HLA-A02:01 | KLK3 | Prostate | 3751 |
| LMLLRLSEPA | HLA-A02:01 | KLK2; KLK3 | Prostate | 3752 |
| HLPQNFLPI | HLA-A02:01 | KLK3 | Prostate | 3753 |
| KLQCVDLHVI | HLA-A02:01 | KLK3 | Prostate | 3754 |
| SLVPWRGGV | HLA-A02:01 | KLK3 | Prostate | 3755 |
| LITELTMPAL | HLA-A02:01 | KLK3 | Prostate | 3756 |
| VLVHPQWVL | HLA-A02:01 | KLK2; | Prostate | 3757 |
| | | KLK3; KLK4 | | |
| LTLAVCGGV | HLA-A02:01 | KLK3 | Prostate | 3758 |
| FLTLAVCGGVLV | HLA-A02:01 | KLK3 | Prostate | 3759 |
| FLPIAQPA | HLA-A02:01 | KLK3 | Prostate | 3760 |
| VFLTLSVTWIA | HLA-A02:01 | KLK3 | Prostate | 3761 |
| LLRLSEPAEL | HLA-A02:01 | KLK3 | Prostate | 3762 |
| TLAVCGGVLV | HLA-A02:01 | KLK3 | Prostate | 3763 |
| VISNDVCAQV | HLA-A02:01 | KLK3 | Prostate | 3764 |
| FMLCAGRWT | HLA-A02:01 | KLK3 | Prostate | 3765 |
| ILITELTMPAL | HLA-A02:01 | KLK3 | Prostate | 3766 |
| FLRPGDDSTL | HLA-A02:01 | KLK3 | Prostate | 3767 |
| RLSEPAEL | HLA-A02:01 | KLK3 | Prostate | 3768 |
| VLTPKKLQCV | HLA-A02:01 | KLK3 | Prostate | 3769 |
| SLYTKVVHY | HLA-A02:01 | KLK3 | Prostate | 3770 |
| ALPERPSL | HLA-A02:01 | KLK3 | Prostate | 3771 |
| ILLGRHSL | HLA-A02:01 | KLK3 | Prostate | 3772 |
| FLTPKKLQC | HLA-A02:01 | KLK3 | Prostate | 3773 |
| SGDSGGPLV | HLA-A02:01 | KLK3 | Prostate | 3774 |
| SLFHPEDTGQV | HLA-A02:01 | KLK3 | Prostate | 3775 |
| RLSEPAELT | HLA-A02:01 | KLK3 | Prostate | 3776 |
| VLHGSLVPW | HLA-A02:01 | KLK3 | Prostate | 3777 |
| GVLVHPQWV | HLA-A02:01 | KLK2; | Prostate | 3778 |
| | | KLK3; KLK4 | | |
| WIAPPLQVL | HLA-A02:01 | KLK3 | Prostate | 3779 |
| TLSVTWIGA | HLA-A02:01 | KLK3 | Prostate | 3780 |
| ELTDAVKV | HLA-A02:01 | KLK3 | Prostate | 3781 |
| FLRPGDDST | HLA-A02:01 | KLK3 | Prostate | 3782 |
| FHPEDTGQV | HLA-A02:01 | KLK3 | Prostate | 3783 |
| TWIAPPLQV | HLA-A02:01 | KLK3 | Prostate | 3784 |
| HPLYDMSLLK | HLA-A03:01 | KLK3 | Prostate | 3785 |
| PLYDMSLLK | HLA-A03:01 | KLK3 | Prostate | 3786 |
| VLTAAHCIRK | HLA-A03:01 | KLK3 | Prostate | 3787 |
| LTAAHCIRK | HLA-A03:01 | KLK3 | Prostate | 3788 |
| SLYTKVVHYRK | HLA-A03:01 | KLK3 | Prostate | 3789 |
| FPHPLYDMSLLK | HLA-A03:01 | KLK3 | Prostate | 3790 |
| SLYTKVVHY | HLA-A03:01 | KLK3 | Prostate | 3791 |
| SLYTKVVHYR | HLA-A03:01 | KLK3 | Prostate | 3792 |
| PHPLYDMSLLK | HLA-A03:01 | KLK3 | Prostate | 3793 |
| KVVHYRKWIK | HLA-A03:01 | KLK2; KLK3 | Prostate | 3794 |
| SIEPEEFLTPK | HLA-A03:01 | KLK3 | Prostate | 3795 |
| ALPERPSLY | HLA-A03:01 | KLK3 | Prostate | 3796 |
| WVLTAAHCIRK | HLA-A03:01 | KLK3 | Prostate | 3797 |
| AVCGGVLVH | HLA-A03:01 | KLK3 | Prostate | 3798 |
| ALPERPSLYTK | HLA-A03:01 | KLK3 | Prostate | 3799 |
| LTAAHCIRK | HLA-A11:01 | KLK3 | Prostate | 3800 |
| HPLYDMSLLK | HLA-A11:01 | KLK3 | Prostate | 3801 |
| STCSVSHPY | HLA-A11:01 | KLK3 | Prostate | 3802 |
| GSIEPEEFLTPK | HLA-A11:01 | KLK3 | Prostate | 3803 |
| SSHDLMLLR | HLA-A11:01 | KLK2; KLK3 | Prostate | 3804 |
| LTAAHCIRNK | HLA-A11:01 | KLK3 | Prostate | 3805 |
| VLTAAHCIRK | HLA-A11:01 | KLK3 | Prostate | 3806 |
| PLYDMSLLK | HLA-A11:01 | KLK3 | Prostate | 3807 |
| SLYTKVVHYR | HLA-A11:01 | KLK3 | Prostate | 3808 |
| GAAPLILSR | HLA-A11:01 | KLK3 | Prostate | 3809 |
| AVCGGVLVH | HLA-A11:01 | KLK3 | Prostate | 3810 |
| GVLQGITSW | HLA-A11:01 | KLK2; KLK3 | Prostate | 3811 |
| STCSWVILI | HLA-A11:01 | KLK3 | Prostate | 3812 |
| RIVGGWECEK | HLA-A11:01 | KLK2; KLK3 | Prostate | 3813 |
| WVLTAAHCIRK | HLA-A11:01 | KLK3 | Prostate | 3814 |
| QVHPQKVTK | HLA-A11:01 | KLK3 | Prostate | 3815 |
| CYASGWGSI | HLA-A24:02 | KLK2; KLK3 | Prostate | 3816 |
| TWIGAAPLI | HLA-A24:02 | KLK3 | Prostate | 3817 |
| VFLTLSVTW | HLA-A24:02 | KLK3 | Prostate | 3818 |
| MWVPVVFLTL | HLA-A24:02 | KLK3 | Prostate | 3819 |
| KFMLCAGRW | HLA-A24:02 | KLK3 | Prostate | 3820 |
| VVFLTLSVTW | HLA-A24:02 | KLK3 | Prostate | 3821 |
| HYRKWIKDTI | HLA-A24:02 | KLK2; KLK3 | Prostate | 3822 |
| VTWIGAAPLI | HLA-A24:02 | KLK3 | Prostate | 3823 |
| TCYASGWGSI | HLA-A24:02 | KLK2; KLK3 | Prostate | 3824 |
| ILLGRHSLF | HLA-A24:02 | KLK3 | Prostate | 3825 |
| VHPQKVTKF | HLA-A24:02 | KLK3 | Prostate | 3826 |
| LFHPEDTGQVF | HLA-A24:02 | KLK3 | Prostate | 3827 |
| VFQVSHSF | HLA-A24:02 | KLK3 | Prostate | 3828 |
| GWGSIEPEEF | HLA-A24:02 | KLK2; KLK3 | Prostate | 3829 |
| FHPEDIGQVF | HLA-A24:02 | KLK3 | Prostate | 3830 |
| YTKVVHYRK | HLA-A30:01 | KLK2; KLK3 | Prostate | 3831 |
| RGRAVCGGV | HLA-A30:01 | KLK3 | Prostate | 3832 |
| VVHYRKWIK | HLA-A30:01 | KLK2; KLK3 | Prostate | 3833 |
| QVHPQKVTK | HLA-A30:01 | KLK3 | Prostate | 3834 |
| KVVHYRKWIK | HLA-A30:01 | KLK2; KLK3 | Prostate | 3835 |
| KVTKFMLCAG | HLA-A30:01 | KLK3 | Prostate | 3836 |
| SSLPHQVPA | HLA-A30:01 | KLK3 | Prostate | 3837 |
| LTMPALPMV | HLA-A30:01 | KLK3 | Prostate | 3838 |
| LTAAHCIRK | HLA-A30:01 | KLK3 | Prostate | 3839 |
| ALPERPSLYTK | HLA-A30:01 | KLK3 | Prostate | 3840 |
| SLYTKVVHY | HLA-A30:01 | KLK3 | Prostate | 3841 |
| VCAQVHPQK | HLA-A30:01 | KLK3 | Prostate | 3842 |
| HSQPWQVLV | HLA-A30:01 | KLK3 | Prostate | 3843 |
| HSFPHPLY | HLA-A30:01 | KLK2; KLK3 | Prostate | 3844 |
| HSFPHPLYD | HLA-A30:01 | KLK3 | Prostate | 3845 |
| LTAAHCIRNK | HLA-A30:01 | KLK3 | Prostate | 3846 |
| SVTWIAPPL | HLA-A30:01 | KLK3 | Prostate | 3847 |
| MSLLKNRFLR | HLA-A33:03 | KLK3 | Prostate | 3848 |
| LYTKVVHYR | HLA-A33:03 | KLK3 | Prostate | 3849 |
| YTKVVHYR | HLA-A33:03 | KLK2; KLK3 | Prostate | 3850 |
| SLYTKVVHYR | HLA-A33:03 | KLK3 | Prostate | 3851 |
| SLLKNRFLR | HLA-A33:03 | KLK3 | Prostate | 3852 |
| VTKFMLCAGR | HLA-A33:03 | KLK3 | Prostate | 3853 |
| YTKVVHYRK | HLA-A33:03 | KLK2; KLK3 | Prostate | 3854 |
| DMSLLKNRFLR | HLA-A33:03 | KLK3 | Prostate | 3855 |
| SSHDLMLLR | HLA-A33:03 | KLK2; KLK3 | Prostate | 3856 |
| YDMSLLKNR | HLA-A33:03 | KLK3 | Prostate | 3857 |
| EALSPPTQH | HLA-A33:03 | KLK3 | Prostate | 3858 |
| GAAPLILSR | HLA-A33:03 | KLK3 | Prostate | 3859 |
| QVLVASRQR | HLA-A33:03 | KLK3 | Prostate | 3860 |
| LPMVLHGSL | HLA-B07:02 | KLK3 | Prostate | 3861 |
| ALPMVLHGSL | HLA-B07:02 | KLK3 | Prostate | 3862 |
| SPDRELGSFL | HLA-B07:02 | KLK3 | Prostate | 3863 |
| MPALPMVLHGSL | HLA-B07:02 | KLK3 | Prostate | 3864 |
| RPSLYTKVV | HLA-B07:02 | KLK3 | Prostate | 3865 |
| LPMVLHGSLV | HLA-B07:02 | KLK3 | Prostate | 3866 |
| SPSILQQSSL | HLA-B07:02 | KLK3 | Prostate | 3867 |
| FPHPLYDMSL | HLA-B07:02 | KLK3 | Prostate | 3868 |
| GPLVCNGVL | HLA-B07:02 | KLK2; KLK3 | Prostate | 3869 |
| QPAPCSQLL | HLA-B07:02 | KLK3 | Prostate | 3870 |
| APSHLPQNF | HLA-B07:02 | KLK3 | Prostate | 3871 |
| QPAPCSQL | HLA-B07:02 | KLK3 | Prostate | 3872 |
| SPDCQAEAL | HLA-B07:02 | KLK3 | Prostate | 3873 |
| MPALPMVL | HLA-B07:02 | KLK3 | Prostate | 3874 |
| VPVVFLTL | HLA-B07:02 | KLK3 | Prostate | 3875 |
| CIRNKSVIL | HLA-B08:01 | KLK3 | Prostate | 3876 |
| LPMVLHGSL | HLA-B08:01 | KLK3 | Prostate | 3877 |
| ILLGRHSL | HLA-B08:01 | KLK3 | Prostate | 3878 |
| LMLLRLSEPA | HLA-B08:01 | KLK2; KLK3 | Prostate | 3879 |
| LVASRGRAV | HLA-B08:01 | KLK3 | Prostate | 3880 |
| ILLGRHSLF | HLA-B08:01 | KLK3 | Prostate | 3881 |
| LMLLRLSEPAEL | HLA-B08:01 | KLK3 | Prostate | 3882 |
| FPHPLYDMSL | HLA-B08:01 | KLK3 | Prostate | 3883 |
| LLKNRFLRPG | HLA-B08:01 | KLK3 | Prostate | 3884 |
| HCIRNKSVIL | HLA-B08:01 | KLK3 | Prostate | 3885 |
| HPLYDMSL | HLA-B08:01 | KLK3 | Prostate | 3886 |
| WVILITEL | HLA-B08:01 | KLK3 | Prostate | 3887 |
| ELGSFLSL | HLA-B08:01 | KLK3 | Prostate | 3888 |
| HPSPDREL | HLA-B08:01 | KLK3 | Prostate | 3889 |
| TPKKLQCV | HLA-B08:01 | KLK3 | Prostate | 3890 |
| VILLGRHSL | HLA-B08:01 | KLK3 | Prostate | 3891 |
| DVLTPKKL | HLA-B08:01 | KLK3 | Prostate | 3892 |
| FLTPKKLQC | HLA-B08:01 | KLK3 | Prostate | 3893 |
| RELGSFLSL | HLA-B13:02 | KLK3 | Prostate | 3894 |
| LIMPALPMV | HLA-B13:02 | KLK3 | Prostate | 3895 |
| STCSWVILI | HLA-B13:02 | KLK3 | Prostate | 3896 |
| LQAHTPSPSI | HLA-B13:02 | KLK3 | Prostate | 3897 |
| RAVCGGVLV | HLA-B13:02 | KLK3 | Prostate | 3898 |
| VTWIGAAPLI | HLA-B13:02 | KLK3 | Prostate | 3899 |
| LQCVDLHVI | HLA-B13:02 | KLK3 | Prostate | 3900 |
| TELTMPALPMV | HLA-B13:02 | KLK3 | Prostate | 3901 |
| HSQPWQVLV | HLA-B13:02 | KLK3 | Prostate | 3902 |
| AQVHPQKV | HLA-B13:02 | KLK3 | Prostate | 3903 |
| QQSSLPHQV | HLA-B13:02 | KLK3 | Prostate | 3904 |
| GVLVHPQWV | HLA-B13:02 | KLK2; | Prostate | 3905 |
| | | KLK3; KLK4 | | |
| ELTDAVKV | HLA-B13:02 | KLK3 | Prostate | 3906 |
| HLPQNFLPI | HLA-B13:02 | KLK3 | Prostate | 3907 |
| SQPWQVLV | HLA-B13:02 | KLK3 | Prostate | 3908 |
| AELTDAVKV | HLA-B13:02 | KLK3 | Prostate | 3909 |
| HSFPHPLYDM | HLA-B46:01 | KLK3 | Prostate | 3910 |
| WIAPPLQVL | HLA-B46:01 | KLK3 | Prostate | 3911 |
| LTMPALPMVL | HLA-B46:01 | KLK3 | Prostate | 3912 |
| QVFQVSHSF | HLA-B46:01 | KLK3 | Prostate | 3913 |
| LTMPALPMV | HLA-B46:01 | KLK3 | Prostate | 3914 |
| STCSVSHPY | HLA-B46:01 | KLK3 | Prostate | 3915 |
| VSHSFPHPL | HLA-B46:01 | KLK2; KLK3 | Prostate | 3916 |
| TMPALPMVL | HLA-B46:01 | KLK3 | Prostate | 3917 |
| HLPQNFLPI | HLA-B46:01 | KLK3 | Prostate | 3918 |
| WIKDTIVAN | HLA-B46:01 | KLK3 | Prostate | 3919 |
| VSHPYSQDL | HLA-B46:01 | KLK3 | Prostate | 3920 |
| SLYTKVVHY | HLA-B46:01 | KLK3 | Prostate | 3921 |
| ALPERPSLY | HLA-B46:01 | KLK3 | Prostate | 3922 |
| GSIEPEEF | HLA-B46:01 | KLK2: KLK3 | Prostate | 3923 |
| VSHPYSQD | HLA-B46:01 | KLK3 | Prostate | 3924 |
| SLPAPLQAH | HLA-B46:01 | KLK3 | Prostate | 3925 |
| TMPALPMVL | HLA-C01:02 | KLK3 | Prostate | 3926 |
| WIAPPLQVL | HLA-C01:02 | KLK3 | Prostate | 3927 |
| VSHSFPHPL | HLA-C01:02 | KLK2; KLK3 | Prostate | 3928 |
| VTWIGAAPL | HLA-C01:02 | KLK3 | Prostate | 3929 |
| HLPQNFLPI | HLA-C01:02 | KLK3 | Prostate | 3930 |
| ITELTMPAL | HLA-C01:02 | KLK3 | Prostate | 3931 |
| LTMPALPMVL | HLA-C01:02 | KLK3 | Prostate | 3932 |
| IAPPLQVLV | HLA-C01:02 | KLK3 | Prostate | 3933 |
| SVTWIAPPL | HLA-C01:02 | KLK3 | Prostate | 3934 |
| ALPERPSL | HLA-C01:02 | KLK3 | Prostate | 3935 |
| WVPVVFLTL | HLA-C01:02 | KLK3 | Prostate | 3936 |
| ASPDCQAEAL | HLA-C01:02 | KLK3 | Prostate | 3937 |
| IAPPLQVL | HLA-C01:02 | KLK3 | Prostate | 3938 |
| AQPAPCSQL | HLA-C01:02 | KLK3 | Prostate | 3939 |
| QHPSPDREL | HLA-C01:02 | KLK3 | Prostate | 3940 |
| DLPTQEPAL | HLA-C01:02 | KLK3 | Prostate | 3941 |
| SLPAPLQAH | HLA-C01:02 | KLK3 | Prostate | 3942 |
| WIAPPLQVL | HLA-C03:04 | KLK3 | Prostate | 3943 |
| VSHSFPHPL | HLA-C03:04 | KLK2; KLK3 | Prostate | 3944 |
| VTWIGAAPL | HLA-C03:04 | KLK3 | Prostate | 3945 |
| SVTWIAPPL | HLA-C03:04 | KLK3 | Prostate | 3946 |
| LTMPALPMV | HLA-C03:04 | KLK3 | Prostate | 3947 |
| LAVCGGVLV | HLA-C03:04 | KLK3 | Prostate | 3948 |
| CALPERPSL | HLA-C03:04 | KLK3 | Prostate | 3949 |
| RAVCGGVLV | HLA-C03:04 | KLK3 | Prostate | 3950 |
| LIMPALPMVL | HLA-C03:04 | KLK3 | Prostate | 3951 |
| IAPPLQVL | HLA-C03:04 | KLK3 | Prostate | 3952 |
| VSHPYSQDL | HLA-C03:04 | KLK3 | Prostate | 3953 |
| LAVCGGVL | HLA-C03:04 | KLK3 | Prostate | 3954 |
| HQVPAPSHL | HLA-C03:04 | KLK3 | Prostate | 3955 |
| WVPVVFLTL | HLA-C03:04 | KLK3 | Prostate | 3956 |
| FHPEDTGQVF | HLA-C04:01 | KLK3 | Prostate | 3957 |
| SFPHPLYDM | HLA-C04:01 | KLK3 | Prostate | 3958 |
| SHSFPHPLY | HLA-C04:01 | KLK2; KLK3 | Prostate | 3959 |
| TMPALPMVL | HLA-C04:01 | KLK3 | Prostate | 3960 |
| CYASGWGSI | HLA-C04:01 | KLK2; KLK3 | Prostate | 3961 |
| LYDMSLLKNRF | HLA-C04:01 | KLK3 | Prostate | 3962 |
| SFLSLPAPL | HLA-C04:01 | KLK3 | Prostate | 3963 |
| WIAPPLQVL | HLA-C04:01 | KLK3 | Prostate | 3964 |
| HLPQNFLPI | HLA-C04:01 | KLK3 | Prostate | 3965 |
| MWVPVVFL | HLA-C04:01 | KLK3 | Prostate | 3966 |
| VFQVSHSF | HLA-C04:01 | KLK3 | Prostate | 3967 |
| SWVILITEL | HLA-C04:01 | KLK3 | Prostate | 3968 |
| RLSEPAEL | HLA-C04:01 | KLK3 | Prostate | 3969 |
| FLSLPAPL | HLA-C04:01 | KLK3 | Prostate | 3970 |
| VMDLPTQE | HLA-C04:01 | KLK3 | Prostate | 3971 |
| SIEPEEFL | HLA-C04:01 | KLK2; KLK3 | Prostate | 3972 |
| HTPSPSIL | HLA-C04:01 | KLK3 | Prostate | 3973 |
| IRNKSVILL | HLA-C07:01 | KLK3 | Prostate | 3974 |
| YRKWIKDTI | HLA-C07:01 | KLK2; KLK3 | Prostate | 3975 |
| HSFPHPLYDM | HLA-C07:01 | KLK3 | Prostate | 3976 |
| LRLSEPAEL | HLA-C07:01 | KLK3 | Prostate | 3977 |
| HSQPWQVLV | HLA-C07:01 | KLK3 | Prostate | 3978 |
| CIRNKSVILL | HLA-C07:01 | KLK3 | Prostate | 3979 |
| GRAVCGGVL | HLA-C07:01 | KLK3 | Prostate | 3980 |
| YRKWIKDTIV | HLA-C07:01 | KLK3 | Prostate | 3981 |
| MSLLKNRFL | HLA-C07:01 | KLK3 | Prostate | 3982 |
| HSFPHPLYDMSL | HLA-C07:01 | KLK3 | Prostate | 3983 |
| HSFPHPLYD | HLA-C07:01 | KLK3 | Prostate | 3984 |
| HSQPWQVL | HLA-C07:01 | KLK3 | Prostate | 3985 |
| KHSQPWQVL | HLA-C07:01 | KLK3 | Prostate | 3986 |
| TPGPDVLIP | HLA-C07:01 | KLK3 | Prostate | 3987 |
| HPEDTGQVE | HLA-C07:01 | KLK3 | Prostate | 3988 |
| HPSPDREL | HLA-C07:01 | KLK3 | Prostate | 3989 |
| IRNKSVIL | HLA-C07:01 | KLK3 | Prostate | 3990 |
| RELGSFLSL | HLA-C07:01 | KLK3 | Prostate | 3991 |
| IRNKSVILL | HLA-C07:02 | KLK3 | Prostate | 3992 |
| YRKWIKDTI | HLA-C07:02 | KLK2; KLK3 | Prostate | 3993 |
| HSFPHPLYDM | HLA-C07:02 | KLK3 | Prostate | 3994 |
| LRLSEPAEL | HLA-C07:02 | KLK3 | Prostate | 3995 |
| TMPALPMVL | HLA-C07:02 | KLK3 | Prostate | 3996 |
| VHPQKVTKF | HLA-C07:02 | KLK3 | Prostate | 3997 |
| VSHSFPHPL | HLA-C07:02 | KLK2; KLK3 | Prostate | 3998 |
| SHSFPHPLY | HLA-C07:02 | KLK2; KLK3 | Prostate | 3999 |
| SFPHPLYDM | HLA-C07:02 | KLK3 | Prostate | 4000 |
| CYASGWGSI | HLA-C07:02 | KLK2; KLK3 | Prostate | 4001 |
| LRPGDDSTL | HLA-C07:02 | KLK3 | Prostate | 4002 |
| IAPPLQVL | HLA-C07:02 | KLK3 | Prostate | 4003 |
| IRNKSVIL | HLA-C07:02 | KLK3 | Prostate | 4004 |
| ALPERPSL | HLA-C07:02 | KLK3 | Prostate | 4005 |
| LYTKVVHY | HLA-C07:02 | KLK3 | Prostate | 4006 |
| ALPERPSLY | HLA-C07:02 | KLK3 | Prostate | 4007 |
| VSEEVCSKLY | HLA-A01:01 | KLK4 | Prostate | 4008 |
| CSKLYDPLY | HLA-A01:01 | KLK4 | Prostate | 4009 |
| LSAAHCFQNSY | HLA-A01:01 | KLK4 | Prostate | 4010 |
| VVSEEVCSKLY | HLA-A01:01 | KLK4 | Prostate | 4011 |
| VLSAAHCFQNSY | HLA-A01:01 | KLK4 | Prostate | 4012 |
| SVVSEEVCSKLY | HLA-A01:01 | KLK4 | Prostate | 4013 |
| SAAHCFQNSY | HLA-A01:01 | KLK4 | Prostate | 4014 |
| ASLSVRHPEY | HLA-A01:01 | KLK4 | Prostate | 4015 |
| SLSVRHPEY | HLA-A01:01 | KLK4 | Prostate | 4016 |
| VSEEVCSKL | HLA-A01:01 | KLK4 | Prostate | 4017 |
| VSESDTIRS | HLA-A01:01 | KLK4 | Prostate | 4018 |
| KLDESVSES | HLA-A01:01 | KLK4 | Prostate | 4019 |
| FTEWIEKT | HLA-A01:01 | KLK4 | Prostate | 4020 |
| SEEVCSKLY | HLA-A01:01 | KLK4 | Prostate | 4021 |
| ESDTIRSIS | HLA-A01:01 | KLK4 | Prostate | 4022 |
| FLGYLILGV | HLA-A02:01 | KLK4 | Prostate | 4023 |
| LLANDLMLI | HLA-A02:01 | KLK4 | Prostate | 4024 |
| FQNSYTIGL | HLA-A02:01 | KLK4 | Prostate | 4025 |
| QMVEASLSV | HLA-A02:01 | KLK4 | Prostate | 4026 |
| MLIKLDESV | HLA-A02:01 | KLK4 | Prostate | 4027 |
| VLQCVNVSV | HLA-A02:01 | KLK4 | Prostate | 4028 |
| LLANGRMPTV | HLA-A02:01 | KLK4 | Prostate | 4029 |
| SQMVEASLSV | HLA-A02:01 | KLK4 | Prostate | 4030 |
| YLILGVAGSL | HLA-A02:01 | KLK4 | Prostate | 4031 |
| WFLGYLILGV | HLA-A02:01 | KLK4 | Prostate | 4032 |
| RMPTVLQCV | HLA-A02:01 | KLK4 | Prostate | 4033 |
| FLGYLILGVA | HLA-A02:01 | KLK4 | Prostate | 4034 |
| YLILGVAGSLV | HLA-A02:01 | KLK4 | Prostate | 4035 |
| LMLIKLDESV | HLA-A02:01 | KLK4 | Prostate | 4036 |
| VLQCVNVSVV | HLA-A02:01 | KLK4 | Prostate | 4037 |
| PLLANDLMLI | HLA-A02:01 | KLK4 | Prostate | 4038 |
| GLLANGRMPTV | HLA-A02:01 | KLK4 | Prostate | 4039 |
| YNRPLLANDLML | HLA-A02:01 | KLK4 | Prostate | 4040 |
| TVLQCVNVSV | HLA-A02:01 | KLK4 | Prostate | 4041 |
| NELFCSGVLV | HLA-A02:01 | KLK4 | Prostate | 4042 |
| KLYDPLYHPS | HLA-A02:01 | KLK4 | Prostate | 4043 |
| MENELFCSGVLV | HLA-A02:01 | KLK4 | Prostate | 4044 |
| RPLLANDLML | HLA-A02:01 | KLK4 | Prostate | 4045 |
| ILGVAGSLV | HLA-A02:01 | KLK4 | Prostate | 4046 |
| MPTVLQCVNVSV | HLA-A02:01 | KLK4 | Prostate | 4047 |
| YLILGVAGS | HLA-A02:01 | KLK4 | Prostate | 4048 |
| ELFCSGVLV | HLA-A02:01 | KLK4 | Prostate | 4049 |
| KLYDPLYHPSM | HLA-A02:01 | KLK4 | Prostate | 4050 |
| KLYDPLYHP | HLA-A02:01 | KLK4 | Prostate | 4051 |
| KLDESVSES | HLA-A02:01 | KLK4 | Prostate | 4052 |
| LLANDLMLIKL | HLA-A02:01 | KLK4 | Prostate | 4053 |
| GLGLHSLEA | HLA-A02:01 | KLK4 | Prostate | 4054 |
| PLLANDLML | HLA-A02:01 | KLK4 | Prostate | 4055 |
| YTIGLGLHSL | HLA-A02:01 | KLK4 | Prostate | 4056 |
| YLQGLVSFG | HLA-A02:01 | KLK4 | Prostate | 4057 |
| KLYDPLYH | HLA-A02:01 | KLK4 | Prostate | 4058 |
| GLLANECL | HLA-A02:01 | KLK4 | Prostate | 4059 |
| YLQGLVSF | HLA-A02:01 | KLK4 | Prostate | 4060 |
| CLVSGWGLL | HLA-A02:01 | KLK4 | Prostate | 4061 |
| TIGLGLHSL | HLA-A02:01 | KLK4 | Prostate | 4062 |
| VLSAAHCFQK | HLA-A03:01 | KLK4 | Prostate | 4063 |
| LLANDLMLIK | HLA-A03:01 | KLK4 | Prostate | 4064 |
| YLQGLVSFGK | HLA-A03:01 | KLK4 | Prostate | 4065 |
| LSAAHCFQK | HLA-A03:01 | KLK4 | Prostate | 4066 |
| SMFCAGGGHDQK | HLA-A03:01 | KLK4 | Prostate | 4067 |
| WVLSAAHCFQK | HLA-A03:01 | KLK4 | Prostate | 4068 |
| KLYDPLYH | HLA-A03:01 | KLK4 | Prostate | 4069 |
| LANDLMLIK | HLA-A03:01 | KLK4 | Prostate | 4070 |
| QWVLSAAHCFQK | HLA-A03:01 | KLK4 | Prostate | 4071 |
| PLLANDLMLIK | HLA-A03:01 | KLK4 | Prostate | 4072 |
| KLYDPLYHP | HLA-A03:01 | KLK4 | Prostate | 4073 |
| IVVSEEVCSK | HLA-A03:01 | KLK4 | Prostate | 4074 |
| NPWGWFLGY | HLA-A03:01 | KLK4 | Prostate | 4075 |
| ATAGNPWGW | HLA-A03:01 | KLK4 | Prostate | 4076 |
| SVSESDTIR | HLA-A03:01 | KLK4 | Prostate | 4077 |
| SVVSEEVCSK | HLA-A03:01 | KLK4 | Prostate | 4078 |
| LSAAHCFQK | HLA-A11:01 | KLK4 | Prostate | 4079 |
| VLSAAHCFQK | HLA-A11:01 | KLK4 | Prostate | 4080 |
| SVVSEEVCSK | HLA-A11:01 | KLK4 | Prostate | 4081 |
| LLANDLMLIK | HLA-A11:01 | KLK4 | Prostate | 4082 |
| LANDLMLIK | HLA-A11:01 | KLK4 | Prostate | 4083 |
| VVSEEVCSK | HLA-A11:01 | KLK4 | Prostate | 4084 |
| YLQGLVSFGK | HLA-A11:01 | KLK4 | Prostate | 4085 |
| LQGLVSFGK | HLA-A11:01 | KLK4 | Prostate | 4086 |
| SMFCAGGGHDQK | HLA-A11:01 | KLK4 | Prostate | 4087 |
| WVLSAAHCFQK | HLA-All:01 | KLK4 | Prostate | 4088 |
| GVPGVYTNLCK | HLA-A11:01 | KLK4 | Prostate | 4089 |
| SVSESDTIR | HLA-A11:01 | KLK4 | Prostate | 4090 |
| ATAGNPWGW | HLA-A11:01 | KLK4 | Prostate | 4091 |
| GVAGSLVSG | HLA-A11:01 | KLK4 | Prostate | 4092 |
| CKFTEWIEK | HLA-A11:01 | KLK4 | Prostate | 4093 |
| QYLQGLVSF | HLA-A24:02 | KLK4 | Prostate | 4094 |
| NGYLQGLVSF | HLA-A24:02 | KLK4 | Prostate | 4095 |
| PWGWPLGYLI | HLA-A24:02 | KLK4 | Prostate | 4096 |
| ICNGYLQGLVSF | HLA-A24:02 | KLK4 | Prostate | 4097 |
| LYDPLYHPSMF | HLA-A24:02 | KLK4 | Prostate | 4098 |
| VYTNLCKFTEW | HLA-A24:02 | KLK4 | Prostate | 4099 |
| CNGYLQGLVSF | HLA-A24:02 | KLK4 | Prostate | 4100 |
| GWFLGYLIL | HLA-A24:02 | KLK4 | Prostate | 4101 |
| KLYDPLYHPSMF | HLA-A24:02 | KLK4 | Prostate | 4102 |
| ALVMENELF | HLA-A24:02 | KLK4 | Prostate | 4103 |
| SYTIGLGLHSL | HLA-A24:02 | KLK4 | Prostate | 4104 |
| SYTIGLGL | HLA-A24:02 | KLK4 | Prostate | 4105 |
| LYDPLYHPSM | HLA-A24:02 | KLK4 | Prostate | 4106 |
| SYTIGLGLH | HLA-A24:02 | KLK4 | Prostate | 4107 |
| VYTNLCKFTE | HLA-A24:02 | KLK4 | Prostate | 4108 |
| LSAAHCFQK | HLA-A30:01 | KLK4 | Prostate | 4109 |
| TIRSISIAS | HLA-A30:01 | KLK4 | Prostate | 4110 |
| VLSAAHCFQK | HLA-A30:01 | KLK4 | Prostate | 4111 |
| LANDLMLIK | HLA-A30:01 | KLK4 | Prostate | 4112 |
| ASLSVRHPE | HLA-A30:01 | KLK4 | Prostate | 4113 |
| RSISIASQCPTA | HLA-A30:01 | KLK4 | Prostate | 4114 |
| AAHCFQNSY | HLA-A30:01 | KLK4 | Prostate | 4115 |
| LVHPQWVLS | HLA-A30:01 | KLK4 | Prostate | 4116 |
| LYHPSMFCA | HLA-A30:01 | KLK4 | Prostate | 4117 |
| KFTEWIEK | HLA-A30:01 | KLK4 | Prostate | 4118 |
| VVSEEVCSK | HLA-A30:01 | KLK4 | Prostate | 4119 |
| RSISIASQC | HLA-A30:01 | KLK4 | Prostate | 4120 |
| SVVSEEVCSK | HLA-A30:01 | KLK4 | Prostate | 4121 |
| CKFTEWIEK | HLA-A30:01 | KLK4 | Prostate | 4122 |
| LCKFTEWIEK | HLA-A30:01 | KLK4 | Prostate | 4123 |
| ASLSVRHPEY | HLA-A30:01 | KLK4 | Prostate | 4124 |
| GVPGVYTNL | HLA-A30:01 | KLK4 | Prostate | 4125 |
| SVRHPEYNR | HLA-A30:01 | KLK4 | Prostate | 4126 |
| SVRHPEYNR | HLA-A33:03 | KLK4 | Prostate | 4127 |
| MVEASLSVR | HLA-A33:03 | KLK4 | Prostate | 4128 |
| LSVRHPEYNR | HLA-A33:03 | KLK4 | Prostate | 4129 |
| SGWGLLANGR | HLA-A33:03 | KLK4 | Prostate | 4130 |
| SLSVRHPEYNR | HLA-A33:03 | KLK4 | Prostate | 4131 |
| QMVEASLSVR | HLA-A33:03 | KLK4 | Prostate | 4132 |
| SVSESDTIR | HLA-A33:03 | KLK4 | Prostate | 4133 |
| ASLSVRHPEYNR | HLA-A33:03 | KLK4 | Prostate | 4134 |
| LSAAHCFQK | HLA-A33:03 | KLK4 | Prostate | 4135 |
| EYNRPLLAN | HLA-A33:03 | KLK4 | Prostate | 4136 |
| ESVSESDTIR | HLA-A33:03 | KLK4 | Prostate | 4137 |
| EWIEKTVQA | HLA-A33:03 | KLK4 | Prostate | 4138 |
| EYNRPLLAND | HLA-A33:03 | KLK4 | Prostate | 4139 |
| TNLCKFTEW | HLA-A33:03 | KLK4 | Prostate | 4140 |
| WFLGYLILG | HLA-A33:03 | KLK4 | Prostate | 4141 |
| HPQWVLSAA | HLA-B07:02 | KLK4 | Prostate | 4142 |
| HPEYNRPLL | HLA-B07:02 | KLK4 | Prostate | 4143 |
| RHPEYNRPLL | HLA-B07:02 | KLK4 | Prostate | 4144 |
| SPHSQPWQAAL | HLA-B07:02 | KLK4 | Prostate | 4145 |
| QPWQAALVM | HLA-B07:02 | KLK4 | Prostate | 4146 |
| SQPWQAALVM | HLA-B07:02 | KLK4 | Prostate | 4147 |
| RPLLANDLM | HLA-B07:02 | KLK4 | Prostate | 4148 |
| CPTAGNSCL | HLA-B07:02 | KLK4 | Prostate | 4149 |
| RPLLANDLML | HLA-B07:02 | KLK4 | Prostate | 4150 |
| NPWGWFLGYL | HLA-B07:02 | KLK4 | Prostate | 4151 |
| SPHSQPWQAA | HLA-B07:02 | KLK4 | Prostate | 4152 |
| RPLLANDL | HLA-B07:02 | KLK4 | Prostate | 4153 |
| HPQWVLSAA | HLA-B08:01 | KLK4 | Prostate | 4154 |
| LMLIKLDESV | HLA-B08:01 | KLK4 | Prostate | 4155 |
| LANGRMPTVL | HLA-B08:01 | KLK4 | Prostate | 4156 |
| YDPLYHPSMF | HLA-B08:01 | KLK4 | Prostate | 4157 |
| TIGLGLHSL | HLA-B08:01 | KLK4 | Prostate | 4158 |
| HPEYNRPL | HLA-B08:01 | KLK4 | Prostate | 4159 |
| YLILGVAGSL | HLA-B08:01 | KLK4 | Prostate | 4160 |
| LANGRMPTV | HLA-B08:01 | KLK4 | Prostate | 4161 |
| LLANGRMPTV | HLA-B08:01 | KLK4 | Prostate | 4162 |
| YLQGLVSF | HLA-B08:01 | KLK4 | Prostate | 4163 |
| NGRMPTVL | HLA-B08:01 | KLK4 | Prostate | 4164 |
| IGLGLHSL | HLA-B08:01 | KLK4 | Prostate | 4165 |
| DLMLIKLD | HLA-B08:01 | KLK4 | Prostate | 4166 |
| LIKLDESV | HLA-B08:01 | KLK4 | Prostate | 4167 |
| EWIEKTVQ | HLA-B08:01 | KLK4 | Prostate | 4168 |
| YLILGVAG | HLA-B08:01 | KLK4 | Prostate | 4169 |
| HPQWVISA | HLA-B08:01 | KLK4 | Prostate | 4170 |
| MENELFCSGVLV | HLA-B13:02 | KLK4 | Prostate | 4171 |
| MENELFCSGV | HLA-B13:02 | KLK4 | Prostate | 4172 |
| WGWFLGYLI | HLA-B13:02 | KLK4 | Prostate | 4173 |
| SQMVEASLSV | HLA-B13:02 | KLK4 | Prostate | 4174 |
| NELFCSGVLV | HLA-B13:02 | KLK4 | Prostate | 4175 |
| SESDTIRSI | HLA-B13:02 | KLK4 | Prostate | 4176 |
| GNPWGWFLGYLI | HLA-B13:02 | KLK4 | Prostate | 4177 |
| MENELFCSGVL | HLA-B13:02 | KLK4 | Prostate | 4178 |
| HCFQNSYTI | HLA-B13:02 | KLK4 | Prostate | 4179 |
| DQEPGSQMV | HLA-B13:02 | KLK4 | Prostate | 4180 |
| RMPTVLQCV | HLA-B13:02 | KLK4 | Prostate | 4181 |
| ELFCSGVLV | HLA-B13:02 | KLK4 | Prostate | 4182 |
| SQPWQAALV | HLA-B13:02 | KLK4 | Prostate | 4183 |
| FLGYLILGV | HLA-B13:02 | KLK4 | Prostate | 4184 |
| GQVGVPGV | HLA-B13:02 | KLK4 | Prostate | 4185 |
| YTIGLGLHSL | HLA-B46:01 | KLK4 | Prostate | 4186 |
| AAHCFQNSY | HLA-B46:01 | KLK4 | Prostate | 4187 |
| FQNSYTIGL | HLA-B46:01 | KLK4 | Prostate | 4188 |
| GQVGVPGVY | HLA-B46:01 | KLK4 | Prostate | 4189 |
| SAAHCFQNSY | HLA-B46:01 | KLK4 | Prostate | 4190 |
| QMVEASLSV | HLA-B46:01 | KLK4 | Prostate | 4191 |
| LANGRMPTV | HLA-B46:01 | KLK4 | Prostate | 4192 |
| YLILGVAGSL | HLA-B46:01 | KLK4 | Prostate | 4193 |
| LLANDLMLI | HLA-B46:01 | KLK4 | Prostate | 4194 |
| CGQVGVPGVY | HLA-B46:01 | KLK4 | Prostate | 4195 |
| TAGNPWGWF | HLA-B46:01 | KLK4 | Prostate | 4196 |
| YLQGLVSF | HLA-B46:01 | KLK4 | Prostate | 4197 |
| KLYDPLYH | HLA-B46:01 | KLK4 | Prostate | 4198 |
| SLSVRHPEY | HLA-B46:01 | KLK4 | Prostate | 4199 |
| TIRSISIAS | HLA-B46:01 | KLK4 | Prostate | 4200 |
| VSGWGLLAN | HLA-B46:01 | KLK4 | Prostate | 4201 |
| VSVVSEEV | HLA-B46:01 | KLK4 | Prostate | 4202 |
| SLVSGSCSQ | HLA-B46:01 | KLK4 | Prostate | 4203 |
| FQNSYTIGL | HLA-C01:02 | KLK4 | Prostate | 4204 |
| GVPGVYTNL | HLA-C01:02 | KLK4 | Prostate | 4205 |
| RMPTVLQCV | HLA-C01:02 | KLK4 | Prostate | 4206 |
| RHPEYNRPL | HLA-C01:02 | KLK4 | Prostate | 4207 |
| LANGRMPTV | HLA-C01:02 | KLK4 | Prostate | 4208 |
| NSYTIGLGL | HLA-C01:02 | KLK4 | Prostate | 4209 |
| YTIGLGLHSL | HLA-C01:02 | KLK4 | Prostate | 4210 |
| LLANDLMLI | HLA-C0L:02 | KLK4 | Prostate | 4211 |
| TIGLGLHSL | HLA-C01:02 | KLK4 | Prostate | 4212 |
| QCPTAGNSCL | HLA-C01:02 | KLK4 | Prostate | 4213 |
| QCPTAGNSC | HLA-C01:02 | KLK4 | Prostate | 4214 |
| YDPLYHPSM | HLA-C01:02 | KLK4 | Prostate | 4215 |
| NRPLLANDL | HLA-C01:02 | KLK4 | Prostate | 4216 |
| KAPCGQVGV | HLA-C01:02 | KLK4 | Prostate | 4217 |
| LANGRMPTVL | HLA-C01:02 | KLK4 | Prostate | 4218 |
| FQNSYTIGL | HLA-C03:04 | KLK4 | Prostate | 4219 |
| LANGRMPTV | HLA-C03:04 | KLK4 | Prostate | 4220 |
| YTIGLGLHSL | HLA-C03:04 | KLK4 | Prostate | 4221 |
| LILGVAGSL | HLA-C03:04 | KLK4 | Prostate | 4222 |
| NSYTIGLGL | HLA-C03:04 | KLK4 | Prostate | 4223 |
| QMVEASLSV | HLA-C03:04 | KLK4 | Prostate | 4224 |
| AALVMENEL | HLA-C03:04 | KLK4 | Prostate | 4225 |
| LANGRMPTVL | HLA-C03:04 | KLK4 | Prostate | 4226 |
| LVSGSCSQI | HLA-C03:04 | KLK4 | Prostate | 4227 |
| AGNPWGWFL | HLA-C03:04 | KLK4 | Prostate | 4228 |
| TAGNPWGWFL | HLA-C03:04 | KLK4 | Prostate | 4229 |
| LYDPLYHIPSM | HLA-C04:01 | KLK4 | Prostate | 4230 |
| LYDPLYHPSMF | HLA-C04:01 | KLK4 | Prostate | 4231 |
| FQNSYTIGL | HLA-C04:01 | KLK4 | Prostate | 4232 |
| KLYDPLYHPSMF | HLA-C04:01 | KLK4 | Prostate | 4233 |
| RMPTVLQCV | HLA-C04:01 | KLK4 | Prostate | 4234 |
| SQPWQAALVM | HLA-C04:01 | KLK4 | Prostate | 4235 |
| SQPWQAALV | HLA-C04:01 | KLK4 | Prostate | 4236 |
| RHPEYNRPLL | HLA-C04:01 | KLK4 | Prostate | 4237 |
| CFQNSYTIGL | HLA-C04:01 | KLK4 | Prostate | 4238 |
| ANDLMLIKL | HLA-C04:01 | KLK4 | Prostate | 4239 |
| LYDPLYHP | HLA-C04:01 | KLK4 | Prostate | 4240 |
| LFCSGVLV | HLA-C04:01 | KLK4 | Prostate | 4241 |
| KLDESVSES | HLA-C04:01 | KLK4 | Prostate | 4242 |
| YLQGLVSF | HLA-C04:01 | KLK4 | Prostate | 4243 |
| VSEEVCSKL | HLA-C04:01 | KLK4 | Prostate | 4244 |
| KLDESVSE | HLA-C04:01 | KLK4 | Prostate | 4245 |
| FQNSYTIGL | HLA-C07:01 | KLK4 | Prostate | 4246 |
| GRMPTVLQCV | HLA-C07:01 | KLK4 | Prostate | 4247 |
| VRHPEYNRPL | HLA-C07:01 | KLK4 | Prostate | 4248 |
| NRPLLANDL | HLA-C07:01 | KLK4 | Prostate | 4249 |
| NSYTIGLGL | HLA-C07:01 | KLK4 | Prostate | 4250 |
| LANGRMPTV | HLA-C07:01 | KLK4 | Prostate | 4251 |
| NRPLLANDLM | HLA-C07:01 | KLK4 | Prostate | 4252 |
| RHPEYNRPL | HLA-C07:01 | KLK4 | Prostate | 4253 |
| SQPWQAALV | HLA-C07:01 | KLK4 | Prostate | 4254 |
| GRMPTVLQC | HLA-C07:01 | KLK4 | Prostate | 4255 |
| AGNPWGWFL | HLA-C07:01 | KLK4 | Prostate | 4256 |
| ANGRMPTVL | HLA-C07:01 | KLK4 | Prostate | 4257 |
| SESDTIRSI | HLA-C07:01 | KLK4 | Prostate | 4258 |
| NPWGWFLGY | HLA-C07:01 | KLK4 | Prostate | 4259 |
| SYTIGLGL | HLA-C07:01 | KLK4 | Prostate | 4260 |
| TEWIEKTVQ | HLA-C07:01 | KLK4 | Prostate | 4261 |
| FQNSYTIGL | HLA-C07:02 | KLK4 | Prostate | 4262 |
| RHPEYNRPL | HLA-C07:02 | \|KLK4 | Prostate | 4263 |
| LYDPLYHPSM | HLA-C07:02 | KLK4 | Prostate | 4264 |
| VRHPEYNRPL | HLA-C07:02 | KLK4 | Prostate | 4265 |
| VHPQWVLSA | HLA-C07:02 | KLK4 | Prostate | 4266 |
| RHPEYNRPLL | HLA-C07:02 | KLK4 | Prostate | 4267 |
| NRPLLANDL | HLA-C07:02 | KLK4 | Prostate | 4268 |
| YNRPLLANDL | HLA-C07:02 | KLK4 | Prostate | 4269 |
| GYLQGLVSF | HLA-C07:02 | KLK4 | Prostate | 4270 |
| YDPLYHPSM | HLA-C07:02 | KLK4 | Prostate | 4271 |
| SYTIGLGL | HLA-C07:02 | KLK4 | Prostate | 4272 |
| VRHPEYNR | HLA-C07:02 | KLK4 | Prostate | 4273 |
| YLQGLVSF | HLA-C07:02 | KLK4 | Prostate | 4274 |
| SSDDKSKSNDPK | HLA-A01:01 | LELP1 | Testis | 4275 |
| SNDPKTEPK | HLA-A01:01 | LELP1 | Testis | 4276 |
| KSNDPKTEPK | HLA-A01:01 | LELP1 | Testis | 4277 |
| SSDDKSKS | HLA-A01:01 | LELP1 | Testis | 4278 |
| SSDDKSKSN | HLA-A01:01 | LELP1 | Testis | 4270 |
| SSDDKSKSND | HLA-A01:01 | LELP1 | Testis | 4280 |
| KLLQRCFEKC | HLA-A02:01 | LELP1 | Testis | 4281 |
| LLQRCFEKC | HLA-A02:01 | LELP1 | Testis | 4282 |
| CLKKLLQRC | HLA-A02:01 | LELP1 | Testis | 4283 |
| CQPSCLKKLL | HLA-A02:01 | LELP1 | Testis | 4284 |
| KLLQRCFEK | HLA-A02:01 | LELP1 | Testis | 4285 |
| CLPCPSQSPS | HLA-A02:01 | LELP1 | Testis | 4286 |
| CQPSCLKKL | HLA-A02:01 | LELP1 | Testis | 4287 |
| CLPCPSQSP | HLA-A02:01 | LELP1 | Testis | 4288 |
| KLLQRCFEKCPW | HLA-A02:01 | LELP1 | Testis | 4289 |
| KCQPSCLKKL | HLA-A02:01 | LELP1 | Testis | 4290 |
| KCPSSCPHA | HLA-A02:01 | LELP1 | Testis | 4291 |
| SCLKKLLQR | HLA-A02:01 | LELP1 | Testis | 4292 |
| KLLQRCFEK | HLA-A03:01 | LELP1 | Testis | 4293 |
| KKLLQRCFEK | HLA-A03:01 | LELP1 | Testis | 4294 |
| KSNDPKTEPK | HLA-A03:01 | LELP1 | Testis | 4295 |
| RCFEKCPWEK | HLA-A03:01 | LELP1 | Testis | 4296 |
| KCQPSCLKK | HLA-A03:01 | LELP1 | Testis | 4297 |
| SSCPPQPCTK | HLA-A11:01 | LELP1 | Testis | 4298 |
| KSNDPKTEPK | HLA-A11:01 | LELP1 | Testis | 4299 |
| KLLQRCFEK | HLA-A11:01 | LELP1 | Testis | 4300 |
| RCFEKCPWEK | HLA-A11:01 | LELP1 | Testis | 4301 |
| SCLKKLLQR | HLA-A11:01 | LELP1 | Testis | 4302 |
| KLLQRCFEKCPW | HLA-A24:02 | LELP1 | Testis | 4303 |
| CQPSCLKKL | HLA-A24:02 | LELP1 | Testis | 4304 |
| LLQRCFEKCPW | HLA-A24:02 | LELP1 | Testis | 4305 |
| KCPAPPKCL | HLA-A24:02 | LELP1 | Testis | 4306 |
| KCQPSCLKKL | HLA-A24:02 | LELP1 | Testis | 4307 |
| SCPPQPCTK | HLA-A24:02 | LELP1 | Testis | 4308 |
| KSKSNDPKTEPK | HLA-A30:01 | LELP1 | Testis | 4309 |
| KSNDPKTEPK | HLA-A30:01 | LELP1 | Testis | 4310 |
| CTKPCPPK | HLA-A30:01 | LELP1 | Testis | 4311 |
| RCFEKCPWEK | HLA-A30:01 | LELP1 | Testis | 4312 |
| KCQPSCLKK | HLA-A30:01 | LELP1 | Testis | 4313 |
| SSCPPQPCTK | HLA-A30:01 | LELP1 | Testis | 4314 |
| ESKCQPSCLK | HLA-A33:03 | LELP1 | Testis | 4315 |
| CLKKLLQR | HLA-A33:03 | LELP1 | Testis | 4316 |
| SCLKKLLQR | HLA-A33:03 | LELP1 | Testis | 4317 |
| PSCLKKLLQR | HLA-A33:03 | LELP1 | Testis | 4318 |
| QPSCLKKLL | HLA-B07:02 | LELP1 | Testis | 4319 |
| CPAPPKCL | HLA-B07:02 | LELP1 | Testis | 4320 |
| CPHACPPPC | HLA-B07:02 | LELP1 | Testis | 4321 |
| CPSQSPSSC | HLA-B07:02 | LELP1 | Testis | 4322 |
| CLKKLLQRCF | HLA-B08:01 | LELP1 | Testis | 4323 |
| ESKCQPSCL | HLA-B08:01 | LELP1 | Testis | 4324 |
| CPWEKCPA | HLA-B08:01 | LELP1 | Testis | 4325 |
| CPAPPKCL | HLA-B08:01 | LELP1 | Testis | 4326 |
| QPSCLKKLL | HLA-B08:01 | LELP1 | Testis | 4327 |
| QPSCLKKL | HLA-B08:01 | LELP1 | Testis | 4328 |
| CESKCQPSCL | HLA-B13:02 | LELP1 | Testis | 4329 |
| CESKCQPSC | HLA-B13:02 | LELP1 | Testis | 4330 |
| LQRCFEKCPW | HLA-B13:02 | LELP1 | Testis | 4331 |
| CQPSCLKKL | HLA-B13:02 | LELP1 | Testis | 4332 |
| CLKKLLQRC | HLA-B13:02 | LELP1 | Testis | 4333 |
| CQPSCLKK | HLA-B13:02 | LELP1 | Testis | 4334 |
| LQRCFEKCPW | HLA-B46:01 | LELP1 | Testis | 4335 |
| CLKKLLQRCF | HLA-B46:01 | LELP1 | Testis | 4336 |
| CQPSCLKKL | HLA-B46:01 | LELP1 | Testis | 4337 |
| KSNDPKTEP | HLA-B46:01 | LELP1 | Testis | 4338 |
| SSDDKSKSN | HLA-B46:01 | LELP1 | Testis | 4339 |
| HACPPPCPP | HLA-B46:01 | LELP1 | Testis | 4340 |
| CQPSCLKKL | HLA-C01:02 | LELP1 | Testis | 4341 |
| KCPAPPKCL | HLA-C01:02 | LELP1 | Testis | 4342 |
| CQPSCLKKLL | HLA-C01:02 | LELP1 | Testis | 4343 |
| KCPSSCPHAC | HLA-C01:02 | LELP1 | Testis | 4344 |
| CQPSCLKKL | HLA-C03:04 | LELP1 | Testis | 4345 |
| CPHACPPPC | HLA-C03:04 | LELP1 | Testis | 4346 |
| CPSQSPSSC | HLA-C03:04 | LELP1 | Testis | 4347 |
| HACPPPCPP | HLA-C03:04 | LELP1 | Testis | 4348 |
| KCPAPPKCL | HLA-C03:04 | LELP1 | Testis | 4349 |
| SSDDKSKSN | HLA-C03:04 | LELP1 | Testis | 4350 |
| CQPSCLKKL | HLA-C04:01 | LELP1 | Testis | 4351 |
| CFEKCPWEK | HLA-C04:01 | LELP1 | Testis | 4352 |
| CQPSCLKKLL | HLA-C04:01 | LELP1 | Testis | 4353 |
| SNDPKTEP | HLA-C04:01 | LELP1 | Testis | 4354 |
| KCPAPPKCL | HLA-C04:01 | LELP1 | Testis | 4355 |
| CQPSCLKKL | HLA-C07:01 | LELP1 | Testis | 4356 |
| LKKLLQRCF | HLA-C07:01 | LELP1 | Testis | 4357 |
| CQPSCLKKLL | HLA-C07:01 | LELP1 | Testis | 4358 |
| KSNDPKTEP | HLA-C07:01 | LELP1 | Testis | 4359 |
| SNDPKTEP | HLA-C07:01 | LELP1 | Testis | 4360 |
| KSNDPKTE | HLA-C07:01 | LELP1 | Testis | 4361 |
| CQPSCLKKL | HLA-C07:02 | LELP1 | Testis | 4362 |
| LKKLLQRCF | HLA-C07:02 | LELP1 | Testis | 4363 |
| CQPSCLKKLL | HLA-C07:02 | LELP1 | Testis | 4364 |
| KCPAPPKCL | HLA-C07:02 | LELP1 | Testis | 4365 |
| SCPPQPCTK | HLA-C07:02 | LELP1 | Testis | 4366 |
| SKCQPSCL | HLA-C07:02 | LELP1 | Testis | 4367 |
| EVDPTSHSY | HLA-A01:01 | MAGEA11 | Lung squam. | 4368 |
| LTQNWVQEKY | HLA-A01:01 | MAGEA11 | Lung squam. | 4369 |
| ETSKMKVLEY | HLA-A01:01 | MAGEA11 | Lung squam. | 4370 |
| IIDLVHLLL | HLA-A01:01 | MAGEA11 | Lung squam. | 4371 |
| KIIDLVHLL | HLA-A02:01 | MAGEA11 | Lung squam. | 4372 |
| KVLEYIANA | HLA-A02:01 | MAGEA11 | Lung squam. | 4373 |
| LLFGIDVKEV | HLA-A02:01 | MAGEA11 | Lung squam. | 4374 |
| VMWEVLSIMGV | HLA-A02:01 | MAGEA11 | Lung squam. | 4375 |
| VLWGPITQI | HLA-A02:01 | MAGEA11 | Lung squam. | 4376 |
| FLFGEPKRL | HLA-A02:01 | MAGEA11 | Lung squam. | 4377 |
| ILHDKIIDLV | HLA-A02:01 | MAGEA11 | Lung squam. | 4378 |
| VMWEVLSIM | HLA-A02:01 | MAGEA11 | Lung squam. | 4379 |
| FLWGPRAHA | HLA-A02:01 | MAGEA11 | Lung squam. | 4380 |
| ILHDKIIDL | HLA-A02:01 | MAGEA11 | Lung squam. | 4381 |
| GLLIIVLGV | HLA-A02:01 | MAGEA11 | Lung squam. | 4382 |
| ALREEGEGV | HLA-A02:01 | MAGEA11 | Lung squam. | 4383 |
| FLFGEPKRLL | HLA-A02:01 | MAGEA11 | Lung squam. | 4384 |
| IDLVHLLLRK | HLA-A03:01 | MAGEA11 | Lung squam. | 4385 |
| LLLRKYRVK | HLA-A03:01 | MAGEA11 | Lung squam. | 4386 |
| GLGCSPASIK | HLA-A03:01 | MAGEA11 | Lung squam. | 4387 |
| RLLTQNWVQEK | HLA-A03:01 | MAGEA11 | Lung squam. | 4388 |
| RKYRVKGLITK | HLA-A03:01 | MAGEA11 | Lung squam. | 4389 |
| KIIDLVHLLLR | HLA-A03:01 | MAGEA11 | Lung squam. | 4390 |
| SIMGVYAGR | HLA-A11:01 | MAGEA11 | Lung squam. | 4391 |
| LTQNWVQEK | HLA-A11:01 | MAGEA11 | Lung squam. | 4392 |
| KAEMLQSVIK | HLA-A11:01 | MAGEA11 | Lung squam. | 4393 |
| RVIMPLEQR | HLA-A11:01 | MAGEA11 | Lung squam. | 4394 |
| VYAGREHFLF | HLA-A24:02 | MAGEA11 | Lung squam. | 4395 |
| LWGPITQIF | HLA-A24:02 | MAGEA11 | Lung squam | 4396 |
| VLWGPITQIF | HLA-A24:02 | MAGEA11 | Lung squam. | 4397 |
| NYEDYFPEI | HLA-A24:02 | MAGEA11 | Lung squam. | 4398 |
| VYAGREHFL | HLA-A24:02 | MAGEA11 | Lung squam. | 4399 |
| KYRVKGLITK | HLA-A30:01 | MAGEA11 | Lung squam. | 4400 |
| MSKVSTMFS | HLA-A30:01 | MAGEA11 | Lung squam. | 4401 |
| RVKGLITKA | HLA-A30:01 | MAGEA11 | Lung squam. | 4402 |
| RVKGLITK | HLA-A30:01 | MAGEAI1 | Lung squam. | 4403 |
| LTQNWVQEK | HLA-A30:01 | MAGEA11 | Lung squam. | 4404 |
| SIMGVYAGR | HLA-A33:03 | MAGEA11 | Lung squam. | 4405 |
| LSIMGVYAGR | HLA-A33:03 | MAGEA11 | Lung squam. | 4406 |
| WVQEKYLVYR | HLA-A33:03 | MAGEA11 | Lung squam. | 4407 |
| EYIANANGR | HLA-A33:03 | MAGEA11 | Lung squam. | 4408 |
| EDYFPEIFR | HLA-A33:03 | MAGEA11 | Lung squam. | 4409 |
| RPADLTRVIM | HLA-B07:02 | MAGEA11 | Lung squam. | 4410 |
| RPADLTRVI | HLA-B07:02 | MAGEA11 | Lung squam. | 4411 |
| RPADLTRVIMPL | HLA-B07:02 | MAGEA11 | Lung squam. | 4412 |
| APYGPQLQW | HLA-B07:02 | MAGEA11 | Lung squam. | 4413 |
| SPSPPQSPQ | HLA-B07:02 | MAGEA11 | Lung squam. | 4414 |
| LLRKYRVKGL | HLA-B08:01 | MAGEA11 | Lung squam. | 4415 |
| HLLLRKYRV | HLA-B08:01 | MAGEA11 | Lung squam. | 4416 |
| LRKYRVKGL | HLA-B08:01 | MAGEA11 | Lung squam. | 4417 |
| FPTVRPADL | HLA-B08:01 | MAGEA11 | Lung squam. | 4418 |
| ILHDKIIDL | HLA-B08:01 | MAGEA11 | Lung squam. | 4419 |
| SGLLIIVL | HLA-B08:01 | MAGEA11 | Lung squam. | 4420 |
| MQLLFGIDV | HLA-B13:02 | MAGEA11 | Lung squam. | 4421 |
| WEVLSIMGV | HLA-B13:02 | MAGEA11 | Lung squam. | 4422 |
| REASVCMQL | HLA-B13:02 | MAGEA11 | Lung squam. | 4423 |
| VLWGPITQI | HLA-B13:02 | MAGEA11 | Lung squam | 4424 |
| SQDILHDKI | HLA-B13:02 | MAGEA11 | Lung squam. | 4425 |
| GLLIIVLGV | HLA-B13:02 | MAGEA11 | Lung squam. | 4426 |
| FQSTERAPY | HLA-B46:01 | MAGEA11 | Lung squam. | 4427 |
| HSYVLVTSL | HLA-B46:01 | MAGEA11 | Lung squam. | 4428 |
| LVTSLNLSY | HLA-B46:01 | MAGEA11 | Lung squam. | 4429 |
| FSPTAMDAIF | HLA-B46:01 | MAGEA11 | Lung squan. | 4430 |
| TSKMKVLEY | HLA-B46:01 | MAGEA11 | Lung squam. | 4431 |
| ILHDKIIDL | HLA-B46:01 | MAGEAI1 | Lung squam. | 4432 |
| FSPTAMDAI | HLA-C01:02 | MAGEA11 | Lung squam. | 4433 |
| SMPKSGLLI | HLA-C01:02 | MAGEA11 | Lung squam. | 4434 |
| AMDAIFGSL | HLA-C01:02 | MAGEA11 | Lung squam. | 4435 |
| RDPTSYPSL | HLA-C01:02 | MAGEA11 | Lung squam. | 4436 |
| RAPYGPQL | HLA-C01:02 | MAGEA11 | Lung squam. | 4437 |
| YVLVTSLNL | HLA-C03:04 | MAGEA11 | Lung squam. | 4438 |
| HSYVLVTSL | HLA-C03:04 | MAGEA11 | Lung squam. | 4439 |
| EAAFFSSTL | HLA-C03:04 | MAGEA11 | Lung squam. | 4440 |
| TAMDAIPGSL | HLA-C03:04 | MAGEA11 | Lung squam. | 4441 |
| AAFFSSTL | HLA-C03:04 | MAGEA11 | Lung squam. | 4442 |
| YEDYFPEIF | HLA-C04:01 | MAGEA11 | Lung squam. | 4443 |
| GTDPACYEF | HLA-C04:01 | MAGEA11 | Lung squam. | 4444 |
| AMDAIFGSL | HLA-C04:01 | MAGEA11 | Lung squam. | 4445 |
| IIDLVHLLL | HLA-C04:01 | MAGEA11 | Lung squam. | 4446 |
| IFREASVCM | HLA-C04:01 | MAGEA11 | Lung squam. | 4447 |
| FREASVCMQL | HLA-C07:01 | MAGEA11 | Lung squam. | 4448 |
| ERAPYGPQL | HLA-C07:01 | MAGEA11 | Lung squam | 4449 |
| LRKYRVKGL | HLA-C07:01 | MAGEA11 | Lung squam. | 4450 |
| KSGLLIIVL | HLA-C07:01 | MAGEA11 | Lung squam. | 4451 |
| RITGGEQVL | HLA-C07:01 | MAGEA11 | Lung squam. | 4452 |
| VYAGREHFL | HLA-C07:02 | MAGEA11 | Lung squam. | 4453 |
| FREASVCMQL | HLA-C07:02 | MAGEA11 | Lung squam. | 4454 |
| IFREASVCM | HLA-C07:02 | MAGEA11 | Lung squam. | 4455 |
| ERAPYGPQL | HLA-C07:02 | MAGEA11 | Lung squam. | 4456 |
| SYVLVTSL | HLA-C07:02 | MAGEA11 | Lung squam. | 4457 |
| LTQDLVQENY | HLA-A01:01 | MAGEA12 | Melanoma | 4458 |
| LVQENYLEY | HLA-A01:01 | MAGEA2; | Melanoma | 4459 |
| | | MAGEA12 | | |
| LLTQDLVQENY | HLA-A01:01 | MAGEA12 | Melanoma | 4460 |
| VVEVVRIGHLY | HLA-A01:01 | MAGEA12 | Melanoma | 4461 |
| MAELVHFLLLKY | HLA-A01:01 | MAGEA12 | Melanoma | 4462 |
| KMAELVHFL | HLA-A02:01 | MAGEA12 | Melanoma | 4463 |
| KMAELVHFLL | HLA-A02:01 | MAGEA12 | Melanoma | 4464 |
| FLWQPRALV | HLA-A02:01 | MAGEA12 | Melanoma | 4465 |
| RKMAELVHFL | HLA-A02:01 | MAGEA12 | Melanoma | 4466 |
| TLVEVTLREV | HLA-A02:01 | MAGEA12 | Melanoma | 4467 |
| YLQLVFGIEV | HLA-A02:01 | MAGEA2; | Melanoma | 4468 |
| | | MAGEA12 | | |
| KMAELVHFLLL | HLA-A02:01 | MAGEA12 | Melanoma | 4469 |
| KIWEELSVL | HLA-A02:01 | MAGEA12 | Melanoma | 4470 |
| LSRKMAELVHFL | HLA-A02:01 | MAGEA12 | Melanoma | 4471 |
| GLLGDNQIV | HLA-A02:01 | MAGEA12 | Melanoma | 4472 |
| ALVETSYVKV | HLA-A02:01 | MAGEA12 | Melanoma | 4473 |
| GLLIIVLAI | HLA-A02:01 | MAGEA2: | Melanoma | 4474 |
| | | MAGEA12 | | |
| FGIEVVEVV | HLA-A02:01 | MAGEA2: | Melanoma | 4475 |
| | | MAGEA12 | | |
| KMAELVHFLLLK | HLA-A03:01 | MAGEA12 | Melanoma | 4476 |
| AELVHFLLLK | HLA-A03:01 | MAGEA12 | Melanoma | 4477 |
| TSFQVALSRK | HLA-A03:01 | MAGEA12 | Melanoma | 4478 |
| RALVETSYVK | HLA-A03:01 | MAGEA12 | Melanoma | 4479 |
| GLLGDNQIVPK | HLA-A03:01 | MAGEA12 | Melanoma | 4480 |
| LLGDNQIVPK | HLA-A03:01 | MAGEA12 | Melanoma | 4481 |
| STLPTTINY | HLA-A03:01 | MAGEA12 | Melanoma | 4482 |
| TSFQVALSRK | HLA-A11:01 | MAGEA12 | Melanoma | 4483 |
| STLPTTINY | HLA-A11:01 | MAGEA12 | Melanoma | 4484 |
| ALVETSYVK | HLA-A11:01 | MAGEA12 | Melanoma | 4485 |
| AELVHFLLLK | HLA-A11:01 | MAGEA12 | Melanoma | 4486 |
| STLVEVTLR | HLA-A11:01 | MAGEA12 | Melanoma | 4487 |
| TSFQVALSR | HLA-A11:01 | MAGEA12 | Melanoma | 4488 |
| SYPPLHEWAF | HLA-A24:02 | MAGEA12 | Melanoma | 4489 |
| SYVKVLHHL | HLA-A24:02 | MAGEA12 | Melanoma | 4490 |
| SYVKVLHHLL | HLA-A24:02 | MAGEA12 | Melanoma | 4491 |
| RAREPFTKA | HLA-A30:01 | MAGEA12 | Melanoma | 4492 |
| KYRAREPFTK | HLA-A30:01 | MAGEA12 | Melanoma | 4493 |
| VVRIGHLYI | HLA-A30:01 | MAGEA12 | Melanoma | 4494 |
| STLPTTINY | HLA-A30:01 | MAGEA12 | Melanoma | 4495 |
| SVFAHPRKL | HLA-A30:01 | MAGEA2; MAGEA12 | Melanoma | 4496 |
| HFLLLKYRAR | HLA-A33:03 | MAGEA2; MAGEA12 | Melanoma | 4497 |
| DFFPVIFSK | HLA-A33:03 | MAGEA2; MAGEA12 | Melanoma | 4498 |
| LVHFLLLKYR | HLA-A33:03 | MAGEA2; MAGEA12 | Melanoma | 4499 |
| STLVEVTLR | HLA-A33:03 | MAGEA12 | Melanoma | 4500 |
| TSFQVALSR | HLA-A33:03 | MAGEA12 | Melanoma | 4501 |
| HPRKLLTQDL | HLA-B07:02 | MAGEA12 | Melanoma | 4502 |
| GPHISYPPL | HLA-B07:02 | MAGEA12 | Melanoma | 4503 |
| LPTTINYTL | HLA-B07:02 | MAGEA12 | Melanoma | 4504 |
| SPQGASTL | HLA-B07:02 | MAGEA12 | Melanoma | 4505 |
| SPSPPHSPQ | HLA-B07:02 | IMAGEA2; MAGEA12 | Melanoma | 4506 |
| LLKYRAREPF | HLA-B08:01 | MAGEA12 | Melanoma | 4507 |
| YVKVLHHLL | HLA-B08:01 | MAGEA12 | Melanoma | 4508 |
| FLLLKYRAREPF | HLA-B08:01 | MAGEA12 | Melanoma | 4509 |
| EPFTKAEML | HLA-B08:01 | MAGEA12 | Melanoma | 4510 |
| EEKIWEEL | HLA-B08:01 | MAGEA2; MAGEA12 | Melanoma | 4511 |
| YVKVLHHL | HLA-B08:01 | MAGEA12 | Melanoma | 4512 |
| SEYLQLVFGI | HLA-B13:02 | MAGEA2; MAGEA12 | Melanoma | 4513 |
| RNPQDFFPV | HLA-B13:02 | MAGEA12 | Melanoma | 4514 |
| YEFLWGPRALV | HLA-B13:02 | MAGEA12 | Melanoma | 4515 |
| GLLIIVLAI | HLA-B13:02 | MAGEA2; MAGEA12 | Melanoma | 4516 |
| KVLHHLLKI | HLA-B13:02 | MAGEA12 | Melanoma | 4517 |
| GLLGDNQIV | HLA-B13:02 | MAGEA12 | Melanoma | 4518 |
| LVQENYLEY | HLA-B46:01 | MAGEA2; MAGEA12 | Melanoma | 4519 |
| SVIRNFQDF | HLA-B46:01 | MAGEA12 | Melanoma | 4520 |
| FQVALSRKM | HLA-B46:01 | MAGEA12 | Melanoma | 4521 |
| ISYPPLHEW | HLA-B46:01 | MAGEA12 | Melanoma | 4522 |
| VIFSKASEY | HLA0B46:01 | MAGEA2; MAGEA12 | Melanoma | 4523 |
| ISGGPHISY | HLA-B46:01 | MAGEA12 | Melanoma | 4524 |
| KMAELVHFL | HLA-C01:02 | MAGEA12 | Melanoma | 4525 |
| HSPQGASTL | HLA-C01:02 | MAGEA12 | Melanoma | 4526 |
| IVPKTGLLI | HLA-C01:02 | MAGEA12 | Melanoma | 4527 |
| TLPTTINYTL | HLA-C01:02 | MAGEA12 | Melanoma | 4528 |
| EGPSTFPDL | HLA-C01:02 | MAGEA12 | Melanoma | 4529 |
| SVFAHPRKL | HLA-C03:04 | MAGEA2; MAGEA12 | Melanoma | 4530 |
| FQVALSRKM | HLA-C03:04 | MAGEA12 | Melanoma | 4531 |
| HSPQGASTL | HLA-C03:04 | MAGEA12 | Melanoma | 4532 |
| ISYPPLHEW | HLA-C03:04 | MAGEA12 | Melanoma | 4533 |
| FAHPRKLL | HLA-C03:04 | MAGEA2; MAGEA12 | Melanoma | 4534 |
| KASEYLQL | HLA-C03:04 | MAGEA2; MAGEA12 | Melanoma | 4535 |
| FQDFFPVIF | HLA-C04:01 | MAGEA12 | Melanoma | 4536 |
| IFSKASEYL | HLA-C04:01 | MAGEA2; MAGEA12 | Melanoma | 4537 |
| TFPDLETSF | HLA-C04:01 | MAGEA12 | Melanoma | 4538 |
| KMAELVHFL | HLA-C04:01 | MAGEA12 | Melanoma | 4539 |
| IWEELSVL | HLA-C04:01 | MAGEA12 | Melanoma | 4540 |
| VRIGHLYIL | HLA-C07:01 | MAGEA12 | Melanoma | 4541 |
| SVFAHPRKL | HLA-C07:01 | MAGEA2; MAGEA12 | Melanoma | 4542 |
| SVFAHPRKLL | HLA-C07:01 | MAGEA2; MAGEA12 | Melanoma | 4543 |
| KMAELVHFL | HLA-C07:01 | MAGEA12 | Melanoma | 4544 |
| KIWEELSVL | HLA-C07:01 | MAGEA12 | Melanoma | 4545 |
| SGGPHISYP | HLA-C07:01 | MAGEA12 | Melanoma | 4546 |
| VRIGHLYIL | HLA-C07:02 | MAGEA12 | Melanoma | 4547 |
| SVFAHPRKL | HLA-C07:02 | MAGEA2; MAGEA12 | Melanoma | 4548 |
| SYVKVLHHL | HLA-C07:02 | MAGEA12 | Melanoma | 4549 |
| SYPPLHEWAF | HLA-C07:02 | MAGEA12 | Melanoma | 4550 |
| LMQDLVQENY | HLA-A01:01 | MAGEA2 | Melanoma | 4551 |
| MQDLVQENY | HLA-A01:01 | MAGEA2 | Melanoma | 4552 |
| ASSFSTTINY | HLA-A01:01 | MAGEA2 | Melanoma | 4553 |
| VVEVVPISHLY | HLA-A01:01 | MAGEA2 | Melanoma | 4554 |
| MVELVHFLLLKY | HLA-A01:01 | MAGEA2 | Melanoma | 4555 |
| KMVELVHFL | HLA-A02:01 | MAGEAZ | Melanoma | 4556 |
| KMVELVHFLL | HLA-A02:01 | MAGEA2 | Melanoma | 4557 |
| RKMVELVHFL | HLA-A02:01 | MAGEA2 | Melanoma | 4558 |
| KIWEELSML | HLA-A02:01 | MAGEA2 | Melanoma | 4559 |
| FLWGPRALI | HLA-A02:01 | MAGEA2 | Melanoma | 4560 |
| ALIETSYVKV | HLA-A02:01 | MAGEA2 | Melanoma | 4561 |
| TLVEVTLGEV | HLA-A02:01 | MAGEA2 | Melanoma | 4562 |
| KMVELVHFLLL | HLA-A02:01 | MAGEA2 | Melanoma | 4563 |
| LLMQDLVQENYL | HLA-A02:01 | MAGEA2 | Melanoma | 4564 |
| ALIETSYV | HLA-A02:01 | MAGEA2 | Melanoma | 4565 |
| GLLGDNQV | HLA-A02:01 | MAGEA2 | Melanoma | 4566 |
| GLLGDNQVM | HLA-A02:01 | MAGEA2 | Melanoma | 4567 |
| TLGEVPAA | HLA-A02:01 | MAGEA2 | Melanoma | 4568 |
| VELVHFLLLK | HLA-A03:01 | MAGEA2 | Melanoma | 4569 |
| KMVELVHFLLLK | HLA-A03:01 | MAGEA2 | Melanoma | 4570 |
| LLGDNQVMPK | HLA-A03:01 | MAGEA2 | Melanoma | 4571 |
| GLLGDNQVMPK | HLA-A03:01 | MAGEA2 | Melanoma | 4572 |
| SSPSTTINY | HLA-A03:01 | MAGEA2 | Melanoma | 4573 |
| TTINYTLWR | HLA-A11:01 | MAGEA2 | Melanoma | 4574 |
| STTINYTLWR | HLA-A11:01 | MAGEA2 | Melanoma | 4575 |
| ALIETSYVK | HLA-A11:01 | MAGEA2 | Melanoma | 4576 |
| SSESTTINY | HLA-ALI:01 | MAGEA2 | Melanoma | 4577 |
| SYVKVLHHTL | HLA-A24:02 | MAGEA2 | Melanoma | 4578 |
| TWEELSMLEVF | HLA-A24:02 | MAGEA2 | Melanoma | 4579 |
| SYVKVLHHTLKI | HLA-A24:02 | MAGEA2 | Melanoma | 4580 |
| MFPDLESEF | HLA-A24:02 | MAGEA2 | Melanoma | 4581 |
| VMPKTGLLI | HLA-A24:02 | MAGEA2 | Melanoma | 4582 |
| KYRAREPVTK | HLA-A30:01 | MAGEA2 | Melanoma | 4583 |
| RAREPVTKA | HLA-A30:01 | MAGEA2 | Melanoma | 4584 |
| RAREPVTK | HLA-A30:01 | MAGEA2 | Melanoma | 4585 |
| SSFSTTINY | HLA-A30:01 | MAGEA2 | Melanoma | 4586 |
| TTINYTLWR | HLA-A33:03 | MAGEA2 | Melanoma | 4587 |
| HISYPPLHER | HLA-A33:03 | MAGEA2 | Melanoma | 4588 |
| SMLEVFEGR | HLA-A33:03 | MAGEA2 | Melanoma | 4589 |
| ISYPPLHER | HLA-A33:03 | MAGEA2 | Melanoma | 4590 |
| EFQAAISR | HLA-A33:03 | MAGEA2 | Melanoma | 4591 |
| HPRKLLMQDL | HLA-B07:02 | MAGEA2 | Melanoma | 4592 |
| VPISHLYIL | HLA-B07:02 | MAGEA2 | Melanoma | 4593 |
| MPKTGLLI | HLA-B07:02 | MAGEA2 | Melanoma | 4594 |
| SPQGASSF | HLA-B07:02 | MAGEA2 | Melanoma | 4595 |
| YPPLHERAL | HLA-B07:02 | MAGEA2 | Melanoma | 4596 |
| YVKVLHHTL | HLA-B08:01 | MAGEA2 | Melanoma | 4597 |
| LLKYRAREPV | HLA-B08:01 | MAGEA2 | Melanoma | 4598 |
| FAHPRKLLM | HLA-B08:01 | MAGEA2 | Melanoma | 4599 |
| AISRKMVEL | HLA-B08:01 | MAGEA2 | Melanoma | 4600 |
| YEFLWGPRALI | HLA-B13:02 | MAGEA2 | Melanoma | 4601 |
| CYEFLWGPRALI | HLA-B13:02 | MAGEA2 | Melanoma | 4602 |
| IEVVEVVPI | HLA-B13:02 | MAGEA2 | Melanoma | 4603 |
| KVLHHTLKI | HLA-B13:02 | MAGEA2 | Melanoma | 4604 |
| RQSDEGSSN | HLA-B13:02 | MAGEA2 | Melanoma | 4605 |
| VLHHTLKI | HLA-B13:02 | MAGEA2 | Melanoma | 4606 |
| FAHPRKLLM | HLA-B46:01 | MAGEA2 | Melanoma | 4607 |
| FQAAISRKM | HLA-B46:01 | MAGEA2 | Melanoma | 4608 |
| SSFSTTINY | HLA-B46:01 | MAGEA2 | Melanoma | 4609 |
| ISYPPLHER | HLA-B46:01 | MAGEA2 | Melanoma | 4610 |
| IGGEPHISY | HLA-B46:01 | MAGEA2 | Melanoma | 4611 |
| FSTTINYTL | HLA-C01:02 | MAGEA2 | Melanoma | 4612 |
| FAHPRKLLM | HLA-C01:02 | MAGEA2 | Melanoma | 4613 |
| VMPKTGLLI | HLA-C01:02 | MAGEA2 | Melanoma | 4614 |
| HSPQGASSF | HLA-C01:02 | MAGEA2 | Melanoma | 4615 |
| YPPLHERAL | HLA-C01:02 | MAGEA2 | Melanoma | 4616 |
| VVPISHLYI | HLA-C01:02 | MAGEA2 | Melanoma | 4617 |
| FAHPRKLLM | HLA-C03:04 | MAGEA2 | Melanoma | 4618 |
| FSTTINYTL | HLA-C03:04 | MAGEA2 | Melanoma | 4619 |
| FQAAISRKM | HLA-C03:04 | MAGEA2 | Melanoma | 4620 |
| AAISRKMVEL | HLA-C03:04 | MAGEA2 | Melanoma | 4621 |
| MFPDLESEF | HLA-C04:01 | MAGEA2 | Melanoma | 4622 |
| CQDFFPVIF | HLA-C04:01 | MAGEA2 | Melanoma | 4623 |
| FAHPRKLLM | HLA-C04:01 | MAGEA2 | Melanoma | 4624 |
| TWEELSML | HLA-C04:01 | MAGEA2 | Melanoma | 4625 |
| FAHPRKLLM | HLA-C07:01 | MAGEA2 | Melanoma | 4626 |
| FQAAISRKM | HLA-C07:01 | MAGEA2 | Melanoma | 4627 |
| FSTTINYTL | HLA-C07:01 | MAGEA2 | Melanoma | 4628 |
| SSFSTTINY | HLA-C07:01 | MAGEA2 | Melanoma | 4629 |
| ARGEALGL | HLA-C07:01 | MAGEA2 | Melanoma | 4630 |
| LEVFEGRED | HLA-C07:01 | MAGEA2 | Melanoma | 4631 |
| FAHPRKLLM | HLA-C07:02 | MAGEA2 | Melanoma | 4632 |
| MFPDLESEF | HLA-C07:02 | MAGEA2 | Melanoma | 4633 |
| FQAAISRKM | HLA-C07:02 | MAGEA2 | Melanoma | 4634 |
| SYPPLHERAL | HLA-C07:02 | MAGEA2 | Melanoma | 4635 |
| ARGEALGL | HLA-C07:02 | MAGEA2 | Melanoma | 4636 |
| SYPPLHER | HLA-C07:02 | MAGEA2 | Melanoma | 4637 |
| EVDPASNTY | HLA-A01:01 | MAGEA4 | Lung squam | 4638 |
| YTLVTCLGLSY | HLA-A01:01 | MAGEA4 | Lung squam, | 4639 |
| KEVDPASNTY | HLA-A01:01 | IMAGEA4 | Lung squam | 4640 |
| VTCLGLSY | HLA-A01:01 | MAGEA2; | Lung squam.; Melanoma | 4641 |
| | | MAGEA12; MAGEA4 | | |
| MLERVIKNY | HLA-A01:01 | MAGEA4 | Lung squam. | 4642 |
| KVLEHVVRV | HLA-A02:01 | MAGEA4 | Lung squam. | 4643 |
| ALLEEEEGV | HLA-A02:01 | MAGEA4 | Lung squam. | 4644 |
| FLWGPRALA | HLA-A02:01 | MAGEA4 | Lung squam | 4645 |
| MIFGIDVKEV | HLA-A02:01 | MAGEA4 | Lung squam | 4646 |
| ALAETSYVKV | HLA-A02:01 | MAGEA4 | Lung squam. | 4647 |
| KVDELAHFL | HLA-A02:01 | MAGEA4 | Lung squam | 4648 |
| FLWGPRALAET | HLA-A02:01 | MAGEA4 | Lung squam. | 4649 |
| ALAETSYV | HLA-A02:01 | IMAGEA4 | Lung squam. | 4650 |
| SLKMIFGIDV | HLA-A02:01 | MAGEA4 | Lung squam. | 4651 |
| ALSNKVDEL | HLA-A02:01 | MAGEA4 | Lung squam. | 4652 |
| GVYDGREHTV | HLA-A02:01 | MAGEA4 | Lung squam. | 4653 |
| PLVPGTLEEV | HLA-A02:01 | MAGEA4 | Lung squam. | 4654 |
| FLWGPRAL | HLA-A02:01 | MAGEA2; | Lung squam.; Melanoma | 4655 |
| | | MAGEA12; MAGEA4 | | |
| SLFREALSNK | HLA-A03:01 | MAGEA4 | Lung squam. | 4656 |
| KVDELAHFLLRK | HLA-A03:01 | MAGEA4 | Lung squam. | 4657 |
| MLERVIKNYK | HLA-A03:01 | MAGEA4 | Lung squam. | 4658 |
| RCFPVIFGK | HLA-A03:01 | MAGEA4 | Lung squam | 4659 |
| GLLGNNQIFPK | HLA-A03:01 | MAGEA4 | Lung squam. | 4660 |
| ELAHFLLRK | HLA-A03:01 | MAGEA4 | Lung squam. | 4661 |
| TTISFTCWR | HLA-A11:01 | MAGEA4 | Lung squam. | 4662 |
| SLFREALSNK | HLA-A11:01 | MAGEA4 | Lung squam. | 4663 |
| ALAETSYVK | HLA-A11:01 | MAGEA4 | Lung squam | 4664 |
| RCFPVIFGK | HLA-A11:01 | MAGEA4 | Lung squam | 4665 |
| GVMGVYDGR | HLA-A11:01 | MAGEA4 | Lung squam. | 4666 |
| NYKRCFPVI | HLA-A24:02 | MAGEA4 | Lung squam. | 4667 |
| NYKRCFPVIF | HLA-A24:02 | MAGEA4 | Lung squam. | 4668 |
| SYVKVLEHV | HLA-A24:02 | MAGEA4 | Lung squam. | 4669 |
| VYGEPRKLL | HLA-A24:02 | MAGEA4 | Lung squam. | 4670 |
| IFPKTGLLI | HLA-A24:02 | MAGEA4 | Lung squam. | 4671 |
| RVRIAYPSL | HLA-A30:01 | MAGEA4 | Lung squam. | 4672 |
| KYRAKELVTK | HLA-A30:01 | MAGEA4 | Lung squam. | 4673 |
| RVRIAYPSLREA | HLA-A30:01 | MAGEA4 | Lung squam. | 4674 |
| RCFPVIFGK | HLA-A30:01 | MAGEA4 | Lung squam. | 4675 |
| KVLEHVVRV | HLA-A30:01 | MAGEA4 | Lung squam. | 4676 |
| TTISFTCWR | HLA-A33:03 | MAGEA4 | Lung squam. | 4677 |
| LAHFLLRKYR | HLA-A33:03 | MAGEA4 | Lung squam. | 4678 |
| PTTISFTCWR | HLA-A33:03 | MAGEA4 | Lung squam. | 4679 |
| ELAHFLLR | HLA-A33:03 | MAGEA4 | Lung squam | 4680 |
| DELAHFLLR | HLA-A33:03 | MAGEA4 | Lung squam | 4681 |
| YPSLREAAL | HLA-B07:02 | MAGEA4 | Lung squam. | 4682 |
| RVRIAYPSL | HLA-B07:02 | MAGEA4 | Lung squam. | 4683 |
| YPSLREAALL | HLA-B07:02 | MAGEA4 | Lung squam | 4684 |
| SPQGASAL | HLA-B07:02 | MAGEA4 | Lung squam. | 4685 |
| SPLVPGTL | HLA-B07:02 | MAGEA4 | Lung squam. | 4686 |
| FLLRKYRAKEL | HLA-B08:01 | MAGEA4 | Lung squam. | 4687 |
| LLRKYRAKEL | HLA-B08:01 | MAGEA4 | Lung squam | 4688 |
| YPSLREAAL | HLA-B08:01 | MAGEA4 | Lung squam. | 4689 |
| ELVTKAEML | HLA-B08:01 | MAGEA4 | Lung squam | 4690 |
| TGLLIVL | HLA-B08:01 | MAGEA2: | Lung squam.; Melanoma | 4691 |
| | | MAGEA12; MAGEA4 | | |
| QIFPKTGL | HLA-B08:01 | MAGEA4 | Lung squam. | 4692 |
| SESLKMIFGI | HLA-B13:02 | MAGEA4 | Lung squam. | 4693 |
| YEFLWGPRAL | HLA-B13:02 | MAGEA2: | Lung squam.; Melanoma | 4694 |
| | | MAGEA12; MAGEA4 | | |
| YEFLWGPRALA | HLA-B13:02 | MAGEA4 | Lung squam. | 4695 |
| GVYDGREHTV | HLA-B13:02 | MAGEA4 | Lung squam | 4696 |
| KVLEHVVRV | HLA-B13:02 | MAGEA4 | Lung squam. | 4697 |
| ALLEEEEGV | HLA-B13:02 | MAGEA4 | Lung squam. | 4698 |
| SALPTTISF | HLA-B46:01 | MAGEA4 | Lung squam | 4699 |
| AAVSSSSPL | HLA-B46:01 | MAGEA4 | Lung squam | 4700 |
| LVTCLGLSY | HLA-B46:01 | MAGEA2: | Lung squam.; Melanoma | 4701 |
| | | MAGEA12; MAGEA4 | | |
| VIKNYKRCF | HLA-B46:01 | MAGEA4 | Lung squam. | 4702 |
| TSYVKVLEH | HLA-B46:01 | MAGEA4 | Lung squam | 4703 |
| KVDELAHFL | HLA-C01:02 | MAGEA4 | Lung squam | 4704 |
| SSPLVPGTL | HLA-C01:02 | MAGEA4 | Lung squam. | 4705 |
| SALPTTISF | HLA-C01:02 | MAGEA4 | Lung squam. | 4706 |
| QSPQGASAL | HLA-C01:02 | MAGEA4 | Lung squam | 4707 |
| TSPDAESL | HLA-C01:02 | MAGEA4 | Lung squam | 4708 |
| AAVSSSSPL | HLA-C03:04 | MAGEA4 | Lung squam, | 4709 |
| SALPTTISF | HLA-C03:04 | MAGEA4 | Lung squam. | 4710 |
| RALAETSYV | HLA-C03:04 | MAGEA4 | Lung squam. | 4711 |
| FLWGPRAL | HLA-C03:04 | MAGEA2; | Lung squam.; Melanoma | 4712 |
| | | MAGEA12; MAGEA4 | | |
| KVDELAHFL | HLA-C04:01 | MAGEA4 | Lung squam. | 4713 |
| IFPKTGLLI | HLA-C04:01 | MAGEA4 | Lung squam. | 4714 |
| VYDGREHTV | HLA-C04:01 | MAGEA4 | Lung squam | 4715 |
| KVDELAHF | HLA-C04:01 | MAGEA4 | Lung squam | 4716 |
| LRKYRAKEL | HLA-C07:01 | MAGEA4 | Lung squam. | 4717 |
| VRVNARVRI | HLA-C07:01 | MAGEA4 | Lung squam | 4718 |
| FREALSNKV | HLA-C07:01 | MAGEA4 | Lung squam | 4719 |
| KTGLLIIVL | HLA-C07:01 | MAGEA2; | Lung squam. | 4720 |
| | | MAGEA12; MAGEA4 | Melanoma | |
| RCFPVIFGK | HLA-C07:01 | MAGEA4 | Lung squam. | 4721 |
| TVYGEPRKL | HLA-C07:01 | MAGEA4 | Lung squam | 4722 |
| YKRCFPVIF | HLA-C07:02 | MAGEA4 | Lung squam. | 4723 |
| LRKYRAKEL | HLA-C07:02 | MAGEA4 | Lung squam. | 4724 |
| VRVNARVRI | HLA-C07:02 | MAGEA4 | Lung squam | 4725 |
| IFPKTGLLI | HLA-C07:02 | MAGEA4 | Lung squam. | 4726 |
| VYGEPRKI | HLA-C07:02 | MAGEA4 | Lung squam. | 4727 |
| VRIAYPSL | HLA-C07:02 | MAGEA4 | Lung squam, | 4728 |
| ASSASSTLY | HLA-A01:01 | MAGEC2 | Melanoma | 4729 |
| LVEFLLLKY | HLA-A01:01 | MAGEC2 | Melanoma | 4730 |
| EASSASSTLY | HLA-A01:01 | MAGEC2 | Melanoma | 4731 |
| VAELVEFLLLKY | HLA-A01:01 | MAGEC2 | Melanoma | 4732 |
| FLAKLNNTV | HLA-A02:01 | MAGEC2 | Melanoma | 4733 |
| VIWEVLNAV | HLA-A02:01 | MAGEC2 | Melanoma | 4734 |
| LLFGLALIEV | HLA-A02:01 | MAGEC2 | Melanoma | 4735 |
| FMELLFGL | HLA-A02:01 | MAGEC2 | Melanoma | 4736 |
| FLLLKYEAEEPV | HLA-A02:01 | MAGEC2 | Melanoma | 4737 |
| YTLDEKVAEL | HLA-A02:01 | MAGEC2 | Melanoma | 4738 |
| VMASESLSV | HLA-A02:01 | MAGEC2 | Melanoma | 4739 |
| KVLEFLAKL | HLA-A02:01 | MAGEC2 | Melanoma | 4740 |
| KVAELVEFL | HLA-A02:01 | MAGEC2 | Melanoma | 4741 |
| ILDEKVAEL | HLA-A02:01 | MAGEC2 | Melanoma | 4742 |
| SLLIILSV | HLA-A02:01 | MAGEC2 | Melanoma | 4743 |
| ALKDVEERV | HLA-A02:01 | MAGEC2 | Melanoma | 4744 |
| TLDEKVAELV | HLA-A02:01 | MAGEC2 | Melanoma | 4745 |
| ILGGPEEEEV | HLA-A02:01 | MAGEC2 | Melanoma | 4746 |
| MLMIVIKYK | HLA-A03:01 | MAGEC2 | Melanoma | 4747 |
| SSFTYTLDEK | HLA-A03:01 | MAGEC2 | Melanoma | 4748 |
| EMLMIVIKYK | HLA-A03:01 | MAGEC2 | Melanoma | 4749 |
| KDYFPVILK | HLA-A03:01 | MAGEC2 | Melanoma | 4750 |
| IILSVIFIK | HLA-A03:01 | MAGEC2 | Melanoma | 4751 |
| SSFTYTLDEK | HLA-ALI:01 | MAGEC2 | Melanoma | 4752 |
| VPSSFPSWYK | HLA-A11:01 | MAGEC2 | Melanoma | 4753 |
| IILSVIFIK | HLA-A11:01 | MAGEC2 | Melanoma | 4754 |
| SSESEESSSQK | HLA-A11:01 | MAGEC2 | Melanoma | 4755 |
| LYLVFSPSSF | HLA-A24:02 | MAGEC2 | Melanoma | 4756 |
| KYKDYFPVI | HLA-A24:02 | MAGEC2 | Melanoma | 4757 |
| KYKDYFPVIL | HLA-A24:02 | MAGEC2 | Melanoma | 4758 |
| VYGEPRELL | HLA-A24:02 | MAGEC2 | Melanoma | 4759 |
| RAHSESIKK | HLA-A30:01 | MAGEC2 | Melanoma | 4760 |
| KYKDYFPVI | HLA-A30:01 | MAGEC2 | Melanoma | 4761 |
| KYKDYFPVILK | HLA-A30:01 | MAGEC2 | Melanoma | 4762 |
| KDYFPVILK | HLA-A30:01 | MAGEC2 | Melanoma | 4763 |
| NAVGVYAGR | HLA-A33:03 | MAGEC2 | Melanoma | 4764 |
| DYFPVILKR | HLA-A33:03 | MAGEC2 | Melanoma | 4765 |
| WVQGHYLEYR | HLA-A33:03 | MAGEC2 | Melanoma | 4766 |
| DYFPVILKRAR | HLA-A33:03 | MAGEC2 | Melanoma | 4767 |
| GPRAHSESI | HLA-B07:02 | MAGEC2 | Melanoma | 4768 |
| FPSWYKDAL | HLA-B07:02 | MAGEC2 | Melanoma | 4769 |
| WGPRAHSESI | HLA-B07:02 | MAGEC2 | Melanoma | 4770 |
| SPSSFSTSSSL | HLA-B07:02 | MAGEC2 | Melanoma | 4771 |
| VPSGVIPNL | HLA-B07:02 | MAGEC2 | Melanoma | 4772 |
| MPPVPGVPF | HLA-B07:02 | MAGEC2 | Melanoma | 4773 |
| SIKKKVLEF | HLA-B08:01 | MAGEC2 | Melanoma | 4774 |
| LKRAREFMEL | HLA-B08:01 | MAGEC2 | Melanoma | 4775 |
| SIKKKVLEFL | HLA-B08:01 | MAGEC2 | Melanoma | 4776 |
| ESIKKKVL | HLA-B08:01 | MAGEC2 | Melanoma | 4777 |
| TLDEKVAEL | HLA-B08:01 | MAGEC2 | Melanoma | 4778 |
| MELLFGLALI | HLA-B13:02 | MAGEC2 | Melanoma | 4779 |
| REFMELLFGL | HLA-B13:02 | MAGEC2 | Melanoma | 4780 |
| MPENSLLIII | HLA-B13:02 | MAGEC2 | Melanoma | 4781 |
| SLLIIILSV | HLA-B13:02 | MAGEC2 | Melanoma | 4782 |
| ALKDVEERV | HLA-B13:02 | MAGEC2 | Melanoma | 4783 |
| ASEEVIWEV | HLA-B13:02 | MAGEC2 | Melanoma | 4784 |
| MASESLSVM | HLA-B46:01 | MAGEC2 | Melanoma | 4785 |
| YLVFSPSSF | HLA-B46:01 | MAGEC2 | Melanoma | 4786 |
| FVYGEPREL | HLA-B46:01 | MAGEC2 | Melanoma | 4787 |
| FSTSSSLIL | HLA-B46:01 | MAGEC2 | Melanoma | 4788 |
| HSSPPYYEF | HLA-B46:01 | MAGEC2 | Melanoma | 4789 |
| SIKKKVLEF | HLA-B46:01 | MAGEC2 | Melanoma | 4790 |
| SVMSSNVSF | HLA-B46:01 | MAGEC2 | Melanoma | 4791 |
| FSTSSSLIL | HLA-C01:02 | MAGEC2 | Melanoma | 4792 |
| SSPPYYEFL | HLA-C01:02 | MAGEC2 | Melanoma | 4793 |
| SSASSTLYL | HLA-C01:02 | MAGEC2 | Melanoma | 4794 |
| FSPSSFSTS | HLA-C01:02 | MAGEC2 | Melanoma | 4795 |
| VGPDHFCVF | HLA-C01:02 | MAGEC2 | Melanoma | 4796 |
| MASESLSVM | HLA-C03:04 | MAGEC2 | Melanoma | 4797 |
| FSTSSSLIL | HLA-C03:04 | MAGEC2 | Melanoma | 4798 |
| FVYGEPREL | HLA-C03:04 | MAGEC2 | Melanoma | 4799 |
| FANTVGLTD | HLA-C03:04 | MAGEC2 | Melanoma | 4800 |
| YKDYFPVIL | HLA-C04:01 | MAGEC2 | Melanoma | 4801 |
| FSTSSSLIL | HLA-C04:01 | MAGEC2 | Melanoma | 4802 |
| HSSPPYYEF | HLA-C04:01 | MAGEC2 | Melanoma | 4803 |
| TLDEKVAEL | HLA-C04:01 | MAGEC2 | Melanoma | 4804 |
| HFCVFANTV | HLA-C04:01 | MAGEC2 | Melanoma | 4805 |
| FVYGEPREL | HLA-C07:01 | MAGEC2 | Melanoma | 4806 |
| KRAREFMEL | HLA-C07:01 | MAGEC2 | Melanoma | 4807 |
| YKDYFPVIL | HLA-C07:01 | MAGEC2 | Melanoma | 4808 |
| HSSPPYYEF | HLA-C07:01 | MAGEC2 | Melanoma | 4809 |
| SSPPYYEF | HLA-C07:01 | MAGEC2 | Melanoma | 4810 |
| KRAREFMEL | HLA-C07:02 | MAGEC2 | Melanoma | 4811 |
| KYKDYFPVI | HLA-C07:02 | MAGEC2 | Melanoma | 4812 |
| FVYGEPREL | HLA-C07:02 | MAGEC2 | Melanoma | 4813 |
| VYGEPREL | HLA-C07:02 | MAGEC2 | Melanoma | 4814 |
| YKDYFPVIL | HLA-C07:02 | MAGEC2 | Melanoma | 4815 |
| HSSPPYYEF | HLA-C07:02 | MAGEC2 | Melanoma | 4816 |
| ISDMLGSLY | HLA-A01:01 | MC2R | Adrenal Gland | 4817 |
| AISDMLGSLY | HLA-A01:01 | MC2R | Adrenal Gland | 4818 |
| LAISDMLGSLY | HLA-A01:01 | MC2R | Adrenal Gland | 4819 |
| ITIFHALRY | HLA-A01:01 | MC2R | Adrenal Gland | 4820 |
| ISDMLGSLYK | HLA-A01:01 | MC2R | Adrenal Gland | 4821 |
| FIFCWAPFV | HLA-A02:01 | MC2R | Adrenal Gland | 4822 |
| SLLGSIFSL | HLA-A02:01 | MC2R | Adrenal Gland | 4823 |
| FIFCWAPFVL | HLA-A02:01 | MC2R | Adrenal Gland | 4824 |
| FVLSLLGSIFSL | HLA-A02:01 | MC2R | Adrenal Gland | 4825 |
| VFIFCWAPFV | HLA-A02:01 | MC2R | Adrenal Gland | 4826 |
| SLFPLMLVFI | HLA-A02:01 | MC2R | Adrenal Gland | 4827 |
| LSLLGSIFSL | HLA-A02:01 | MC2R | Adrenal Gland | 4828 |
| FIYAFRSPEL | HLA-A02:01 | MC2R | Adrenal Gland | 4829 |
| LQAPMYFFI | HLA-A02:01 | MC2R | Adrenal Gland | 4830 |
| SLFPLMLV | HLA-A02:01 | MC2R | Adrenal Gland | 4831 |
| GVLENLIVL | HLA-A02:01 | MC2R | Adrenal Gland | 4832 |
| VLENLIVLL | HLA-A02:01 | MC2R | Adrenal Gland | 4833 |
| SLYKILENI | HLA-A02:01 | MC2R | Adrenal Gland | 4834 |
| SLFQVNGML | HLA-A02:01 | MC2R | Adrenal Gland | 4835 |
| VLPEEIFFT | HLA-A02:01 | MC2R | Adrenal Gland | 4836 |
| ILRNMGYLK | HLA-A03:01 | MC2R | Adrenal Gland | 4837 |
| IILRNMGYLK | HLA-A03:01 | MC2R | Adrenal Gland | 4838 |
| ISDMLGSLYK | HLA-A03:01 | MC2R | Adrenal Gland | 4839 |
| AISDMLGSLYK | HLA-A03:01 | MC2R | Adrenal Gland | 4840 |
| AVIDPFIYA | HLA-A03:01 | MC2R | Adrenal Gland | 4841 |
| VLLAVFKNK | HLA-A03:01 | MC2R | Adrenal Gland | 4842 |
| ISDMLGSLYK | HLA-A11:01 | MC2R | Adrenal Gland | 4843 |
| STLPRANMK | HLA-A11:01 | MC2R | Adrenal Gland | 4844 |
| AISDMLGSLYK | HLA-A11:01 | MC2R | Adrenal Gland | 4845 |
| AVIDPFIYAFR | HLA-A11:01 | MC2R | Adrenal Gland | 4846 |
| AVIDPFIYA | HLA-A11:01 | MC2R | Adrenal Gland | 4847 |
| RYITIFHAL | HLA-A24:02 | MC2R | Adrenal Gland | 4848 |
| VFIFCWAPF | HLA-A24:02 | MC2R | Adrenal Gland | 4849 |
| PYCACYMSLF | HLA-A24:02 | MC2R | Adrenal Gland | 4850 |
| IYAFRSPEL | HLA-A24:02 | MC2R | Adrenal Gland | 4851 |
| VVLTVIWTF | HLA-A24:02 | MC2R | Adrenal Gland | 4852 |
| ILRNMGYLK | HLA-A30:01 | MC2R | Adrenal Gland | 4853 |
| RSHTRKIST | HLA-A30:01 | MC2R | Adrenal Gland | 4854 |
| HTRKISTLP | HLA-A30:01 | MC2R | Adrenal Gland | 4855 |
| AVIDPFIYA | HLA-A30:01 | MC2R | Adrenal Gland | 4856 |
| VLLAVFKNK | HLA-A30:01 | MC2R | Adrenal Gland | 4857 |
| RYTTIFHAL | HLA-A30:01 | MC2R | Adrenal Gland | 4858 |
| YITIFHALR | HLA-A33:03 | MC2R | Adrenal Gland | 4859 |
| MFLLARSHTR | HLA-A33:03 | MC2R | Adrenal Gland | 4860 |
| RYITIFHALR | HLA-A33:03 | MC2R | Adrenal Gland | 4861 |
| HTRKISTLPR | HLA-A33:03 | MC2R | Adrenal Gland | 4862 |
| DAFKKMIFCSR | HLA-A33:03 | MC2R | Adrenal Gland | 4863 |
| RYHSIVTMR | HLA-A33:03 | MC2R | Adrenal Gland | 4864 |
| ENILILR | HLA-A33:03 | MC2R | Adrenal Gland | 4865 |
| LPRANMKGAI | HLA-B07:02 | MC2R | Adrenal Gland | 4866 |
| APMYFFICSL | HLA-B07:02 | MC2R | Adrenal Gland | 4867 |
| LPRANMKGAITL | HLA-B07:02 | MC2R | Adrenal Gland | 4868 |
| VPTVITFTSL | HLA-B07:02 | MC2R | Adrenal Gland | 4869 |
| APFVLHVLL | HLA-B07:02 | MC2R | Adrenal Gland | 4870 |
| YLKPRGSF | HLA-B08:01 | MC2R | Adrenal Gland | 4871 |
| TMRRTVVVL | HLA-B08:01 | MC2R | Adrenal Gland | 4872 |
| HALRYHSI | HLA-B08:01 | MC2R | Adrenal Gland | 4873 |
| YITIFHAL | HLA-B08:01 | MC2R | Adrenal Gland | 4874 |
| DAFKKMIF | HLA-B08:01 | MC2R | Adrenal Gland | 4875 |
| TVITFTSI | HLA-B08:01 | MC2R | Adrenal Gland | 4876 |
| LQAPMYFFI | HLA-B13:02 | MC2R | Adrenal Gland | 4877 |
| EEIFFTISI | HLA-B13:02 | MC2R | Adrenal Gland | 4878 |
| SDMLGSLYKI | HLA-B13:02 | MC2R | Adrenal Gland | 4879 |
| SLYKILENI | HLA-B13:02 | MC2R | Adrenal Gland | 4880 |
| RRTVVVLTV | HLA-B13:02 | MC2R | Adrenal Gland | 4881 |
| FTISIVGVL | HLA-B46:01 | MC2R | Adrenal Gland | 4882 |
| FQVNGMILIM | HLA-B46:01 | MC2R | Adrenal Gland | 4883 |
| MTFCPSNPY | HLA-B46:01 | MC2R | Adrenal Gland | 4884 |
| VIDPFIYAF | HLA-B46:01 | MC2R | Adrenal Gland | 4885 |
| VLHVLLMTF | HLA-B46:01 | MC2R | Adrenal Gland | 4886 |
| YLKPRGSF | HLA-B46:01 | MC2R | Adrenal Gland | 4887 |
| IAADRYITI | HLA-C01:02 | MC2R | Adrenal Gland | 4888 |
| WAPFVLHVL | HLA-C01:02 | MC2R | Adrenal Gland | 4889 |
| ITFTSLFPL | HLA-C01:02 | MC2R | Adrenal Gland | 4890 |
| FTSLFPLML | HLA-C01:02 | MC2R | Adrenal Gland | 4891 |
| RSPELRDAF | HLA-C01:02 | MC2R | Adrenal Gland | 4892 |
| FCPSNPYCAC | HLA-C01:02 | MC2R | Adrenal Gland | 4893 |
| FTISIVGVL | HLA-C03:04 | MC2R | Adrenal Gland | 4894 |
| FSHHVPTVI | HLA-C03:04 | MC2R | Adrenal Gland | 4895 |
| IAADRYITI | HLA-C03:04 | MC2R | Adrenal Gland | 4896 |
| YAFRSPEL | HLA-C03:04 | MC2R | Adrenal Gland | 4897 |
| FIYAFRSPEL | HLA-C03:04 | MC2R | Adrenal Gland | 4898 |
| ISDMLGSLY | HLA-C04:01 | MC2R | Adrenal Gland | 4899 |
| LFQVNGMLI | HLA-C04:01 | MC2R | Adrenal Gland | 4900 |
| TFTSLFPLM | HLA-C04:01 | MC2R | Adrenal Gland | 4901 |
| LFPLMLVFI | HLA-C04:01 | MC2R | Adrenal Gland | 4902 |
| VIDPFIYAF | HLA-C04:01 | MC2R | Adrenal Gland | 4903 |
| IFCWAPFVL | HLA-C04:01 | MC2R | Adrenal Gland | 4904 |
| LRYHSIVTM | HLA-C07:01 | MC2R | Adrenal Gland | 4905 |
| RRTVVVLTV | HLA-C07:01 | MC2R | Adrenal Gland | 4906 |
| FHALRYHSI | HLA-C07:01 | MC2R | Adrenal Gland | 4907 |
| MRRTVVVL | HLA-C07:01 | MC2R | Adrenal Gland | 4908 |
| HHVPTVITF | HLA-C07:01 | MC2R | Adrenal Gland | 4909 |
| DRYITIFHA | HLA-C07:01 | MC2R | Adrenal Gland | 4910 |
| LRYHSIVTM | HLA-C07:02 | MC2R | Adrenal Gland | 4911 |
| LYAFRSPEL | HLA-C07:02 | MC2R | Adrenal Gland | 4912 |
| FHALRYHSI | HLA-C07:02 | MC2R | Adrenal Gland | 4913 |
| HHVPTVITF | HLA-C07:02 | MC2R | Adrenal Gland | 4914 |
| MRRTVVVL | HLA-C07:02 | MC2R | Adrenal Gland | 4915 |
| FSGNQVWRY | HLA-A01:01 | MMP13 | Head & neck | 4916 |
| WSDVTPLNF | HLA-A01:01 | MMP13 | Head & neck | 4917 |
| FAERYLRSYY | HLA-A01:01 | MMP13 | Head & neck | 4918 |
| LSEEDLQFAERY | HLA-A01:01 | MMP13 | Head & neck | 4919 |
| FLTKSFWPEL | HLA-A02:01 | MMP13 | Head & neck | 4920 |
| YLFFQRTHTV | HLA-A02:01 | MMP13 | Head & neck | 4492 |
| RLIEEDFPGI | HLA-A02:01 | MMP13 | Head & neck | 4922 |
| SLWSSWDYRI | HLA-A02:01 | MMP13 | Head & neck | 4923 |
| SLSLDAITSL | HLA-A02:01 | MMP13 | Head & neck | 4924 |
| MQSFFGLEV | HLA-A02:01 | MMP13 | Head & neck | 4925 |
| LMFPIYTYT | HLA-A02:01 | MMP13 | Head & neck | 4926 |
| KLDDNTLDV | HLA-A02:01 | MMP13 | Head & neck | 4927 |
| SLWSSWDYRLYL | HLA-A02:01 | MMP13 | Head & neck | 4928 |
| FKVWSDVTPL | HLA-A02:01 | MMP13 | Head & neck | 4929 |
| TLLFSGNQV | HLA-A02:01 | MMP13 | Head & neck | 4930 |
| RLHDGIADI | HLA-A02:01 | MMP13 | Head & neck | 4931 |
| SIWSNRIVRV | HLA-A02:01 | MMP13 | Head & neck | 4932 |
| GIGDKVDAV | HLA-A02:01 | MMP13 | Head & neck | 4933 |
| SLRGETMIFK | HLA-A03:01 | MMP13 | Head & neck | 4934 |
| LMFPIYTYTGK | HLA-A03:01 | MMP13 | Head & neck | 4935 |
| LIFIFRGRK | HLA-A03:01 | MMP13 | Head & neck | 4936 |
| KISELGLPK | HLA-A03:01 | MMP13 | Head & neck | 4937 |
| RVMPANSILWC | HLA-A03:01 | MMP13 | Head & neck | 4938 |
| KISELGLPK | HLA-A11:01 | MMP13 | Head & neck | 4939 |
| MIFKDRFFWR | HLA-A11:01 | MMP13 | Head & neck | 4940 |
| KSFWPELPNR | HLA-A11:01 | MMP13 | Head & neck | 4941 |
| SIWSNRIVR | HLA-A11:01 | MMP13 | Head & neck | 4942 |
| NVFPRTLKWSK | HLA-A11:01 | MMP13 | Head & neck | 4943 |
| IYFFNGPIQF | HLA-A24:02 | MMP13 | Head & neck | 4944 |
| YFFNGPIQF | HLA-A24:02 | MMP13 | Head & neck | 4945 |
| SWDYRLYLF | HLA-A24:02 | MMP13 | Head & neck | 4946 |
| VWSDVTPLNF | HLA-A24:02 | MMP13 | Head & neck | 4947 |
| RSYYHPINLA | HLA-A30:01 | MMP13 | Head & neck | 4948 |
| RSYYHPTNL | HLA-A30:01 | MMP13 | Head & neck | 4949 |
| RGRKFWALN | HLA-A30:01 | MMP13 | Head & neck | 4950 |
| LTKSFWPEL | HLA-A30:01 | MMP13 | Head & neck | 4951 |
| HAFPPGPNY | HLA-A30:01 | MMP13 | Head & neck | 4952 |
| SLRGETMIFK | HLA-A30:01 | MMP13 | Head & neck | 4953 |
| MIFKDRFFWR | HLA-A33:03 | MMP13 | Head & neck | 4954 |
| HIMDKDYPR | HLA-A33:03 | MMP13 | Head & neck | 4955 |
| IFKDRFFWR | HLA-A33:03 | MMP13 | Head & neck | 4956 |
| DLIFIFRGR | HLA-A33:03 | MMP13 | Head & neck | 4957 |
| DYRLYLFFQR | HLA-A33:03 | MMP13 | Head & neck | 4958 |
| EYSIWSNRIVR | HLA-A33:03 | MMP13 | Head & neck | 4959 |
| FPRTLKWSKM | HLA-B07:02 | MMP13 | Head & neck | 4960 |
| HPTNLAGIL | HLA-B07:02 | MMP13 | Head & neck | 4961 |
| RVMPANSIL | HLA-B07:02 | MMP13 | Head & neck | 4962 |
| YPFDGPSGL | HLA-B07:02 | MMP13 | Head & neck | 4963 |
| YPFDGPSGLL | HLA-B07:02 | MMP13 | Head & neck | 4964 |
| HPQQVDAEL | HLA-B07:02 | MMP13 | Head & neck | 4965 |
| FRGRKFWAL | HLA-B08:01 | MMP13 | Head & neck | 4966 |
| FIFRGRKFWAL | HLA-B08:01 | MMP13 | Head & neck | 4967 |
| WTHCRALPL | HLA-B08:01 | MMP13 | Head & neck | 4968 |
| YPKKISEL | HLA-B08:01 | MMP13 | Head & neck | 4969 |
| TLKWSKMNL | HLA-B08:01 | MMP13 | Head & neck | 4970 |
| EGYPKKISE | HLA-B08:01 | MMP13 | Head & neck | 4971 |
| REMQSFFGL | HLA-B13:02 | MMP13 | Head & neck | 4972 |
| ADIMISFGI | HLA-B13:02 | MMP13 | Head & neck | 4973 |
| FEYSIWSNRI | HLA-B13:02 | MMP13 | Head & neck | 4974 |
| RLHDGIADI | HLA-B13:02 | MMP13 | Head & neck | 4975 |
| GLPKEVKKI | HLA-B13:02 | MMP13 | Head & neck | 4976 |
| MQSFFGLEV | HLA-B13:02 | MMP13 | Head & neck | 4977 |
| YTYTGKSHF | HLA-B46:01 | MMP13 | Head & neck | 4978 |
| YFFNGPIQF | HLA-B46:01 | MMP13 | Head & neck | 4979 |
| WTHCRALPL | HLA-B46:01 | MMP13 | Head & neck | 4980 |
| HAFPPGPNY | HLA-B46:01 | MMP13 | Head & neck | 4981 |
| GIADIMISF | HLA-B46:01 | MMP13 | Head & neck | 4982 |
| ALMFPIYTY | HLA-B46:01 | MMP13 | Head & neck | 4983 |
| SSWDYRLYL | HLA-C01:02 | MMP13 | Head & neck | 4984 |
| RVMPANSIL | HLA-C01:02 | MMP13 | Head & neck | 4985 |
| RSYYHPTNL | HLA-C01:02 | MMP13 | Head & neck | 4986 |
| VTPLNFTRL | HLA-C01:02 | MMP13 | Head & neck | 4987 |
| YTPDMTHSEV | HLA-C01:02 | MMP13 | Head & neck | 4988 |
| VMPANSIL | HLA-C01:02 | MMP13 | Head & neck | 4989 |
| LSLDAITSI | HLA-C03:04 | MMP13 | Head & neck | 4990 |
| RVMPANSIL | HLA-C03:04 | MMP13 | Head & neck | 4991 |
| WTHCRALPL | HLA-C03:04 | MMP13 | Head & neck | 4992 |
| AAYEHPSHDL | HLA-C03:04 | MMP13 | Head & neck | 4993 |
| LSWTHCRAL | HLA-C03:04 | MMP13 | Head & neck | 4994 |
| RYDDTNHIM | HLA-C04:01 | MMP13 | Head & neck | 4995 |
| WRYDDTNHIM | HLA-C04:01 | MMP13 | Head & neck | 4996 |
| YPFDGPSGLL | HLA-C04:01 | MMP13 | Head & neck | 4997 |
| SWDYRLYLF | HLA-C04:01 | MMP13 | Head & neck | 4998 |
| FFNGPIQF | HLA-C04:01 | MMP13 | Head & neck | 4999 |
| WRYDDTNHI | HLA-C07:01 | MMP13 | Head & neck | 5000 |
| FRGRKFWAL | HLA-C07:01 | MMP13 | Head & neck | 5001 |
| YFFNGPIQF | HLA-C07:01 | MMP13 | Head & neck | 5002 |
| RSYYHPTNL | HLA-C07:01 | MMP13 | Head & neck | 5003 |
| SSWDYRLYL | HLA-C07:01 | MMP13 | Head & neck | 5004 |
| FNGPIQFEY | HLA-C07:01 | MMP13 | Head & neck | 5005 |
| YFFNGPIQF | HLA-C07:02 | MMP13 | Head & neck | 5006 |
| FRGRKFWAL | HLA-C07:02 | MMP13 | Head & neck | 5007 |
| LFFQRTHTV | HLA-C07:02 | MMP13 | Head & neck | 5008 |
| NRIDAAYEH | HLA-C07:02 | MMP13 | Head & neck | 5009 |
| GYPKKISEL | HLA-C07:02 | MMP13 | Head & neck | 5010 |
| PTDNQGTDV | HLA-A01:01 | PAGE5 | Melanoma | 5011 |
| PTDNQGTDVEAF | HLA-A01:01 | PAGE5 | Melanoma | 5012 |
| GTDVEAFQQELA | HLA-A01:01 | PAGE5 | Melanoma | 5013 |
| VLEAGEGQL | HLA-A01:01 | PAGE5 | Melanoma | 5014 |
| GTDVEAFQQ | HLA-A01:01 | PAGE5 | Melanoma | 5015 |
| FQQELALLKI | HLA-A02:01 | PAGE5 | Melanoma | 5016 |
| TLPTFDPTKV | HLA-A02:01 | PAGE5 | Melanoma | 5017 |
| FQQELALL | HLA-A02:01 | PAGE5 | Melanoma | 5018 |
| VLEAGEGQL | HLA-A02:01 | PAGE5 | Melanoma | 5019 |
| GTLPTFDPTKV | HLA-A02:01 | PAGE5 | Melanoma | 5020 |
| AFQQELALLKI | HLA-A02:01 | PAGE5 | Melanoma | 5021 |
| KVLEAGEGQL | HLA-A02:01 | PAGE5 | Melanoma | 5022 |
| EAFQQELALLKI | HLA-A02:01 | PAGE5 | Melanoma | 5023 |
| TLPTFDPTKVL | HLA-A02:01 | PAGE5 | Melanoma | 5024 |
| FQQELALLK | HLA-A02:01 | PAGE5 | Melanoma | 5025 |
| GWAGTREEV | HLA-A02:01 | PAGE5 | Melanoma | 5026 |
| IKNEGAPAV | HLA-A02:01 | PAGE5 | Melanoma | 5027 |
| AVQGTDVEA | HLA-A02:01 | PAGE5 | Melanoma | 5028 |
| ALLKIEDA | HLA-A02:01 | PAGE5 | Melanoma | 5029 |
| GTLPTFDPTK | HLA-A03:01 | PAGE5 | Melanoma | 5030 |
| AFQQELALLK | HLA-A03:01 | PAGE5 | Melanoma | 5031 |
| GIAPSGEIK | HLA-A03:01 | PAGE5 | Melanoma | 5032 |
| FQQELALLK | HLA-A03:01 | PAGE5 | Melanoma | 5033 |
| IVQQPTEEK | HLA-A03:01 | PAGE5 | Melanoma | 5034 |
| GTLPTFDPTK | HLA-A11:01 | PAGE5 | Melanoma | 5035 |
| GIAPSGEIK | HLA-A11:01 | PAGE5 | Melanoma | 5036 |
| IVQQPTEEK | HLA-A11:01 | PAGE5 | Melanoma | 5037 |
| FQQELALLK | HLA-A11:01 | PAGE5 | Melanoma | 5038 |
| AFQQELALL | HLA-A24:02 | PAGE5 | Melanoma | 5039 |
| VEAFQQELALL | HLA-A24:02 | PAGE5 | Melanoma | 5040 |
| EAFQQELALL | HLA-A24:02 | PAGE5 | Melanoma | 5041 |
| VREGTLPTF | HLA-A24:02 | PAGE5 | Melanoma | 5042 |
| VQGTDVEAF | HLA-A24:02 | PAGE5 | Melanoma | 5043 |
| GTLPTFDPTK | HLA-A30:01 | PAGE5 | Melanoma | 5044 |
| VTRSQSSER | HLA-A30:01 | PAGE5 | Melanoma | 5045 |
| SSQPVGPVI | HLA-A30:01 | PAGE5 | Melanoma | 5046 |
| PTFDPTKVL | HLA-A30:01 | PAGE5 | Melanoma | 5047 |
| GTREEVRDM | HLA-A30:01 | PAGE5 | Melanoma | 5048 |
| IVQQPTEEK | HLA-A30:01 | PAGE5 | Melanoma | 5049 |
| VTRSQSSER | HLA-A33:03 | PAGE5 | Melanoma | 5050 |
| EVRDMSEHVTR | HLA-A33:03 | PAGE5 | Melanoma | 5051 |
| MQAPWAGNR | HLA-A33:03 | PAGE5 | Melanoma | 5052 |
| DMSEHVTR | HLA-A33:03 | PAGE5 | Melanoma | 5053 |
| APAVQGTDV | HLA-B07:02 | PAGE5 | Melanoma | 5054 |
| LPTFDPTKVL | HLA-B07:02 | PAGE5 | Melanoma | 5055 |
| APWAGNRGWA | HLA-B07:02 | PAGE5 | Melanoma | 5056 |
| GPDVREGTL | HLA-B07:02 | PAGE5 | Melanoma | 5057 |
| WAGNRGWAG | HLA-B08:01 | PAGE5 | Melanoma | 5058 |
| EAFQQELAL | HLA-B08:01 | PAGE5 | Melanoma | 5059 |
| WAGNRGWAGT | HLA-B08:01 | PAGE5 | Melanoma | 5060 |
| LALLKIED | HLA-B08:01 | PAGE5 | Melanoma | 5061 |
| DPTKVLEA | HLA-B08:01 | PAGE5 | Melanoma | 5062 |
| ELALLKIED | HLA-B08:01 | PAGE5 | Melanoma | 5063 |
| FQQELALLKI | HLA-B13:02 | PAGE5 | Melanoma | 5064 |
| SSQPVGPVI | HLA-B13:02 | PAGE5 | Melanoma | 5065 |
| QQELALLKI | HLA-B13:02 | PAGE5 | Melanoma | 5066 |
| SQPVGPVIV | HLA-B13:02 | PAGE5 | Melanoma | 5067 |
| SSQPVGPVI | HLA-B46:01 | PAGE5 | Melanoma | 5068 |
| EAFQQELAL | HLA-B46:01 | PAGE5 | Melanoma | 5069 |
| GTREEVRDM | HLA-B46:01 | PAGE5 | Melanoma | 5070 |
| DVREGTLPTF | HLA-B46:01 | PAGE5 | Melanoma | 5071 |
| VQGTDVEAF | HLA-B46:01 | PAGE5 | Melanoma | 5072 |
| EVRDMSEH | HLA-B46:01 | PAGE5 | Melanoma | 5073 |
| SSQPVGPVI | HLA-C01:02 | PAGE5 | Melanoma | 5074 |
| VLEAGEGQL | HLA-C01:02 | PAGE5 | Melanoma | 5075 |
| AFQQELALL | HLA-C01:02 | PAGE5 | Melanoma | 5076 |
| TLPTFDPTKVL | HLA-C01:02 | PAGE5 | Melanoma | 5077 |
| TLPTFDPTKV | HLA-C01:02 | PAGE5 | Melanoma | 5078 |
| DAPGDGPDV | HLA-C01:02 | PAGE5 | Melanoma | 5079 |
| SSQPVGPVI | HLA-C03:04 | PAGE5 | Melanoma | 5080 |
| EAFQQELAL | HLA-C03:04 | PAGE5 | Melanoma | 5081 |
| VEAFQQELAL | HLA-C03:04 | PAGE5 | Melanoma | 5082 |
| PTFDPTKVL | HLA-C03:04 | PAGE5 | Melanoma | 5083 |
| FQQELALLK | HLA-C04:01 | PAGE5 | Melanoma | 5084 |
| AFQQELALL | HLA-C04:01 | PAGE5 | Melanoma | 5085 |
| VREGTLPTF | HLA-C04:01 | PAGE5 | Melanoma | 5086 |
| TFDPTKVL | HLA-C04:01 | PAGE5 | Melanoma | 5087 |
| TFDPTKVLE | HLA-C04:01 | PAGE5 | Melanoma | 5088 |
| TFDPTKVLEA | HLA-C04:01 | PAGE5 | Melanoma | 5089 |
| VREGTLPTF | HLA-C07:01 | PAGE5 | Melanoma | 5090 |
| SSQPVGPVI | HLA-C07:01 | PAGE5 | Melanoma | 5091 |
| FQQELALLKI | HLA-C07:01 | PAGE5 | Melanoma | 5092 |
| TREEVRDM | HLA-C07:01 | PAGE5 | Melanoma | 5093 |
| KRQEEEPPTD | HLA-C07:01 | PAGE5 | Melanoma | 5094 |
| GDGPDVREG | HLA-C07:01 | PAGE5 | Melanoma | 5095 |
| VREGTLPTF | HLA-C07:02 | PAGE5 | Melanoma | 5096 |
| AFQQELALL | HLA-C07:02 | PAGE5 | Melanoma | 5097 |
| SSQPVGPVI | HLA-C07:02 | PAGE5 | Melanoma | 5098 |
| TREEVRDM | HLA-C07:02 | PAGE5 | Melanoma | 5099 |
| FQQELALL | HLA-C07:02 | PAGE5 | Melanoma | 5100 |
| FLMKKELDY | HLA-A01:01 | PGK2 | Testis | 5101 |
| MIIGGGMAY | HLA-A01:01 | PGK2 | Testis | 5102 |
| VADKIQLIK | HLA-A01:01 | PGK2 | Testis | 5103 |
| ASIPSIKY | HLA-A01:01 | PGK2 | Testis | 5104 |
| NMEIGASLF | HLA-A01:01 | PGK2 | Testis | 5105 |
| ALMDEIVKA | HLA-A02:01 | PGK2 | Testis | 5106 |
| FLMKKELDYFA | HLA-A02:01 | PGK2 | Testis | 5107 |
| YSLAPVAVEL | HLA-A02:01 | PGK2 | Testis | 5108 |
| SLAPVAVEL | HLA-A02:01 | PGK2 | Testis | 5109 |
| IVWNGPLGV | HLA-A02:01 | PGK2 | Testis | 5110 |
| FLKDCVGAEV | HLA-A02:01 | PGK2 | Testis | 5111 |
| VIMRVDFNV | HLA-A02:01 | PGK2 | Testis | 5112 |
| GFLMKKELDYFA | HLA-A02:01 | PGK2 | Testis | 5113 |
| GMAYTFLKV | HLA-A02:01 | PGK2 | Testis | 5114 |
| KILPGVEAL | HLA-A02:01 | PGK2 | Testis | 5115 |
| CLDNGAKAV | HLA-A02:01 | PGK2 | Testis | 5116 |
| KLDVRGKRV | HLA-A02:01 | PGK2 | Testis | 5117 |
| LLEGKILPGV | HLA-A02:01 | PGK2 | Testis | 5118 |
| ELLEGKILPGV | HLA-A02:01 | PGK2 | Testis | 5119 |
| RVDFNVPMKK | HLA-A03:01 | PGK2 | Testis | 5120 |
| IMRVDFNVPMKK | HLA-A03:01 | PGK2 | Testis | 5121 |
| RIKASIPSIK | HLA-A03:01 | PGK2 | Testis | 5122 |
| KVADKIQLIK | HLA-A03:01 | PGK2 | Testis | 5123 |
| GVFEWDAPAK | HLA-A03:01 | PGK2 | Testis | 5124 |
| SLAPVAVELK | HLA-A03:01 | PGK2 | Testis | 5125 |
| GVFEWDAFAK | HLA-A11:01 | PGK2 | Testis | 5126 |
| GQMAYTFLK | HLA-A11:01 | PGK2 | Testis | 5127 |
| KASGFLMKK | HLA-A11:01 | PGK2 | Testis | 5128 |
| KVADKIQLIK | HLA-A11:01 | PGK2 | Testis | 5129 |
| VWNGPLGVF | HLA-A24:02 | PGK2 | Testis | 5130 |
| KYSLAPVAV | HLA-A24:02 | PGK2 | Testis | 5131 |
| IGGGMAYTF | HLA-A24:02 | PGK2 | Testis | 5132 |
| VWNGPLGVFEW | HLA-A24:02 | PGK2 | Testis | 5133 |
| VRITFPVDF | HLA-A24:02 | PGK2 | Testis | 5134 |
| RIKASIPSI | HLA-A30:01 | PGK2 | Testis | 5135 |
| RIKASIPSIK | HLA-A30:01 | PGK2 | Testis | 5136 |
| KASGELMKK | HLA-A30:01 | PGK2 | Testis | 5137 |
| SNKNHAQVV | HLA-A30:01 | PGK2 | Testis | 5138 |
| KGQDPSGKK | HLA-A30:01 | PGK2 | Testis | 5139 |
| IVKDIMAK | HLA-A30:01 | PGK2 | Testis | 5140 |
| HAQVVAQAR | HLA-A33:03 | PGK2 | Testis | 5141 |
| FAKALENPVR | HLA-A33:03 | PGK2 | Testis | 5142 |
| DVRGKRVIMR | HLA-A33:03 | PGK2 | Testis | 5143 |
| LTLDKLDVR | HLA-A33:03 | PGK2 | Testis | 5144 |
| LPHKASGFL | HLA-B07:02 | PGK2 | Testis | 5145 |
| NPVRPFLAI | HLA-B07:02 | PGK2 | Testis | 5146 |
| NPVRPFLAIL | HLA-B07:02 | PGK2 | Testis | 5147 |
| NPAPGSVIL | HLA-B07:02 | PGK2 | Testis | 5148 |
| NPAPGSVILL | HLA-B07:02 | PGK2 | Testis | 5149 |
| APVAVELKSL | HLA-B07:02 | PGK2 | Testis | 5150 |
| MSLSKKLTL | HLA-B08:01 | PGK2 | Testis | 5151 |
| FLMKKELDYF | HLA-B08:01 | PGK2 | Testis | 5152 |
| SLSKKLTL | HLA-B08:01 | PGK2 | Testis | 5153 |
| SGFLMIKKEL | HLA-B08:01 | PGK2 | Testis | 5154 |
| LENPVRPFLAI | HLA-B13:02 | PGK2 | Testis | 5155 |
| LEGKILPGV | HLA-B13:02 | PGK2 | Testis | 5156 |
| LDYFAKALENPV | HLA-B13:02 | PGK2 | Testis | 5157 |
| GQDPSGKKI | HLA-B13:02 | PGK2 | Testis | 5158 |
| ALMDEIVKA | HLA-B13:02 | PGK2 | Testis | 5159 |
| GMAYTFLKV | HLA-B13:02 | PGK2 | Testis | 5160 |
| MIIGGGMAY | HLA-B46:-1 | PGK2 | Testis | 5161 |
| MAYTFLKVL | HLA-B46:01 | PGK2 | Testis | 5162 |
| FAKGTKALM | HLA-B46:01 | PGK2 | Testis | 5163 |
| AEPDKIEAF | HLA-B46:01 | PGK2 | Testis | 5164 |
| SLAPVAVEL | HLA-B46:01 | PGK2 | Testis | 5165 |
| SLAPVAVEL | HLA-C01:02 | PGK2 | Testis | 5166 |
| MAYTFLKVL | HLA-C01:02 | PGK2 | Testis | 5167 |
| YSLAPVAVEL | HLA-C01:02 | PGK2 | Testis | 5168 |
| ILPGVEAL | HLA-C01:02 | PGK2 | Testis | 5169 |
| LAPVAVEL | HLA-C01:02 | PGK2 | Testis | 5170 |
| ISPGWMGL | HLA-C01:02 | PGK2 | Testis | 5171 |
| MAYTFLKVL | HLA-C03:04 | PGK2 | Testis | 5172 |
| YSLAPVAVEL | HLA-C03:04 | PGK2 | Testis | 5173 |
| MSLSKKLTL | HLA-C03:04 | PGK2 | Testis | 5174 |
| FAKGTKALM | HLA-C03:04 | PGK2 | Testis | 5175 |
| FAKGTKAL | HLA-C03:04 | PGK2 | Testis | 5176 |
| VWNGPLGVF | HLA-C04:01 | PGK2 | Testis | 5177 |
| LENPVRPFL | HLA-C04:01 | PGK2 | Testis | 5178 |
| NMEIGASLF | HLA-C04:01 | PGK2 | Testis | 5179 |
| KFDENAQV | HLA-C04:01 | PGK2 | Testis | 5180 |
| LAPVAVEL | HLA-C04:01 | PGK2 | Testis | 5181 |
| VRITFPVDF | HLA-C07:01 | PGK2 | Testis | 5182 |
| MAYTFLKVL | HLA-C07:01 | PGK2 | Testis | 5183 |
| VRITFPVDFV | HLA-C07:01 | PGK2 | Testis | 5184 |
| SLAPVAVEL | HLA-C07:01 | PGK2 | Testis | 5185 |
| KRVIMRVD | HLA-C07:01 | PGK2 | Testis | 5186 |
| LENPVRPF | HLA-C07:01 | PGK2 | Testis | 5187 |
| VRITFPVDF | HLA-C07:02 | PGK2 | Testis | 5188 |
| MAYTFLKVL | HLA-C07:02 | PGK2 | Testis | 5189 |
| MSLSKKLTL | HLA-C07:02 | PGK2 | Testis | 5190 |
| VRPFLAIL | HLA-C07:02 | PGK2 | Testis | 5191 |
| SLAPVAVEL | HLA-C07:02 | PGK2 | Testis | 5192 |
| VTDMCKTEY | HLA-A01:01 | PNLIPRP1 | Pancreas | 5193 |
| WVTDMCKTEY | HLA-A01:01 | PNLIPRP1 | Pancreas | 5194 |
| MLDILLTEY | HLA-A01:01 | PNLIPRP1 | Pancreas | 5195 |
| MLDILLVKY | HLA-A01:01 | PNLIPRP1 | Pancreas | 5196 |
| MLIFWTITL | HLA-A02:01 | PNLIPRP1 | Pancreas | 5197 |
| AQMLDILLV | HLA-A02:01 | PNLIPRP1 | Pancreas | 5198 |
| GIWAGQVLPV | HLA-A02:01 | PNLIPRP1 | Pancreas | 5199 |
| FLWNNNVINPTL | HLA-A02:01 | PNLIPRP1 | Pancreas | 5200 |
| LLTEYSYPPSKV | HLA-A02:01 | PNLIPRP1 | Pancreas | 5201 |
| KLFEVEEV | HLA-A02:01 | PNLIPRP1 | Pancreas | 5202 |
| QMLDILLV | HLA-A02:01 | PNLIPRP1 | Pancreas | 5203 |
| LIFWTITLFL | HLA-A02:01 | PNLIPRP1 | Pancreas | 5204 |
| MLIFWTITLFL | HLA-A02:01 | PNLIPRP1 | Pancreas | 5205 |
| VINPTLPKV | HLA-A02:01 | PNLIPRP1 | Pancreas | 5206 |
| KLDVGTIEKV | HLA-A02:01 | PNLIPRP1 | Pancreas | 5207 |
| KLFEVEEVNC | HLA-A02:01 | PNLIPRP1 | Pancreas | 5208 |
| LLLSDPSTI | HLA-A02:01 | PNLIPRP1 | Pancreas | 5209 |
| ILNPDGFAA | HLA-A02:01 | PNLIPRP1 | Pancreas | 5210 |
| RLDPSDADFV | HLA-A02:01 | PNLIPRP1 | Pancreas | 5211 |
| GLDPVEASF | HLA-A02:01 | PNLIPRP1 | Pancreas | 5212 |
| AAYPCTSYK | HLA-A03:01 | PNLIPRP1 | Pancreas | 5213 |
| KTEYSYPPSK | HLA-A03:01 | PNLIPRP1 | Pancreas | 5214 |
| LVKYSYPPSK | HLA-A03:01 | PNLIPRP1 | Pancreas | 5215 |
| FAAYPCTSYK | HLA-A03:01 | PNLIPRP1 | Pancreas | 5216 |
| NVINPTLPK | HLA-A03:01 | PNLIPRP1 | Pancreas | 5217 |
| KIGTRFLLY | HLA-A03:01 | PNLIPRP1 | Pancreas | 5218 |
| AAYPCTSYK | HLA-A11:01 | PNLIPRP1 | Pancreas | 5219 |
| NVINPTLPK | HLA-A11:01 | PNLIPRP1 | Pancreas | 5220 |
| STHSYEFDAK | HLA-A11:01 | PNLIPRP1 | Pancreas | 5221 |
| KILPWSPEK | HLA-ALL:01 | PNLIPRP1 | Pancreas | 5222 |
| AYPCTSYKSF | HLA-A24:02 | PNLIPRP1 | Pancreas | 5223 |
| SYKYYLESI | HLA-A24:02 | PNLIPRP1 | Pancreas | 5224 |
| SYPPSKVHLI | HLA-A24:02 | PNLIPRP1 | Pancreas | 5225 |
| SYPPSKVHL | HLA-A24:02 | PNLIPRP1 | Pancreas | 5226 |
| YYLESILNPDGF | HLA-A24:02 | PNLIPRP1 | Pancreas | 5227 |
| AAYPCTSYK | HLA-A30:01 | PNLIPRP1 | Pancreas | 5228 |
| RSRMPTDGS | HLA-A30:01 | PNLIPRP1 | Pancreas | 5229 |
| KTEYSYPPSK | HLA-A30:01 | PNLIPRP1 | Pancreas | 5230 |
| KVKFLWNNN | HLA-A30:01 | PNLIPRP1 | Pancreas | 5231 |
| KILPWSPEK | HLA-A30:01 | PNLIPRP1 | Pancreas | 5232 |
| SYKSFESDK | HLA-A30:01 | PNLIPRP1 | Pancreas | 5233 |
| NTHQYSIFR | HLA-A33:03 | PNLIPRP1 | Pancreas | 5234 |
| HYADKFAGR | HLA-A33:03 | PNLIPRP1 | Pancreas | 5235 |
| DFVACNHLR | HLA-A33:03 | PNLIPRP1 | Pancreas | 5236 |
| EASNFARWR | HLA-A33:03 | PNLIPRP1 | Pancreas | 5237 |
| EPWGGTAIR | HLA-A33:03 | PNLIPRP1 | Pancreas | 5238 |
| MPGCKKNAL | HLA-B07:02 | PNLIPRP1 | Pancreas | 5239 |
| TPGLSRITGL | HLA-B07:02 | PNLIPRP1 | Pancreas | 5240 |
| SPEKIGTRFL | HLA-B07:02 | PNLIPRP1 | Pancreas | 5241 |
| APLIPFLQF | HLA-B07:02 | PNLIPRP1 | Pancreas | 5242 |
| NPNNFQILL | HLA-B07:02 | PNLIPRP1 | Pancreas | 5243 |
| MPGCKKNAL | HLA-B08:01 | PNLIPRP1 | Pancreas | 5244 |
| HLRSYKYYL | HLA-B08:01 | PNLIPRP1 | Pancreas | 5245 |
| FARWRYGVSI | HLA-B08:01 | PNLIPRP1 | Pancreas | 5246 |
| TGQIKVAL | HLA-B08:01 | PNLIPRP1 | Pancreas | 5247 |
| YPPSKVHL | HLA-B08:01 | PNLIPRP1 | Pancreas | 5248 |
| SDADFVDVI | HLA-B13:02 | PNLIPRP1 | Pancreas | 5249 |
| AQMLDILLV | HLA-B13:02 | PNLIPRP1 | Pancreas | 5250 |
| YNFCSEDTV | HLA-B13:02 | PNLIPRP1 | Pancreas | 5251 |
| HQYSIFRGI | HLA-B13:02 | PNLIPRP1 | Pancreas | 5252 |
| TQAANNVRV | HLA-B13:02 | PNLIPRP1 | Pancreas | 5253 |
| FAAYPCTSY | HLA-B46:01 | PNLIPRP1 | Pancreas | 5254 |
| FVACNHLRSY | HLA-B46:01 | PNLIPRP1 | Pancreas | 5255 |
| YSYPPSKVH | HLA-B46:01 | PNLIPRP1 | Pancreas | 5256 |
| ILKPGSTHSY | HLA-B46:01 | PNLIPRP1 | Pancreas | 5257 |
| VACNHLRSY | HLA-B46:01 | PNLIPRP1 | Pancreas | 5258 |
| AAYPCTSY | HLA-B46:01 | PNLIPRP1 | Pancreas | 5259 |
| YSYPPSKVHL | HLA-C01:02 | PNLIPRP1 | Pancreas | 5260 |
| VLPVSRSRM | HLA-C01:02 | PNLIPRP1 | Pancreas | 5261 |
| FFPNGGESM | HLA-C01:02 | PNLIPRP1 | Pancreas | 5262 |
| SMPGCKKNAL | HLA-C01:02 | PNLIPRP1 | Pancreas | 5263 |
| YSYPPSKVHL | HLA-C03:04 | PNLIPRP1 | Pancreas | 5264 |
| HSYEFDAKI | HLA-C03:04 | PNLIPRP1 | Pancreas | 5265 |
| FAAYPCTSY | HLA-C03:04 | PNLIPRP1 | Pancreas | 5266 |
| YSYPPSKVH | HLA-C03:04 | PNLIPRP1 | Pancreas | 5267 |
| TAIRPLKIL | HLA-C03:04 | PNLIPRP1 | Pancreas | 5268 |
| FFPNGGESM | HLA-C04:01 | PNLIPRP1 | Pancreas | 5269 |
| RLDPSDADF | HLA-C04:01 | PNLIPRP1 | Pancreas | 5270 |
| GLDPVEASF | HLA-C04:01 | PNLIPRP1 | Pancreas | 5271 |
| SYPPSKVHL | HLA-C04:01 | PNLIPRP1 | Pancreas | 5272 |
| IFWTITLFL | HLA-C04:01 | PNLIPRP1 | Pancreas | 5273 |
| SFESTPEEV | HLA-C04:01 | PNLIPRP1 | Pancreas | 5274 |
| WRYGVSITL | HLA-C07:01 | PNLIPRP1 | Pancreas | 5275 |
| VREDTLLTL | HLA-C07:01 | PNLIPRP1 | Pancreas | 5276 |
| ARWRYGVSI | HLA-C07:01 | PNLIPRP1 | Pancreas | 5277 |
| SRMPTDGSL | HLA-C07:01 | PNLIPRP1 | Pancreas | 5278 |
| RSYKYYLES | HLA-C07:01 | PNLIPRP1 | Pancreas | 5279 |
| WRYGVSITL | HLA-C07:02 | PNLIPRP1 | Pancreas | 5280 |
| FFPNGGESM | HLA-C07:02 | PNLIPRP1 | Pancreas | 5281 |
| VREDTLLTL | HLA-C07:02 | PNLIPRP1 | Pancreas | 5282 |
| SYPPSKVHL | HLA-C07:02 | PNLIPRP1 | Pancreas | 5283 |
| SRMPTDGSL | HLA-C07:02 | PNLIPRP1 | Pancreas | 5284 |
| GTDPNIPDEY | HLA-A01:01 | POTEG; POTEE; | Colorectal; Prostate; | 5285 |
| | | POTEH | nc | |
| LMAKALLLY | HLA-A01:01 | POTEG; POTEE; | Colorectal; Prostate; | 5286 |
| | | POTEH | Uterine | |
| SDVDIRKDLY | HLA-A01:01 | POTEE | Colorectal; Uterine | 5287 |
| LSDYKEKQMPKY | HLA-A01:01 | POTEE | Colorectal; Uterine | 5288 |
| MKSDVDIRKDLY | HLA-A01:01 | POTEE | Colorectal; Uterine | 5289 |
| KSDVDIRKDLY | HLA-A01:01 | POTEE | Colorectal; Uterine | 5290 |
| FPDTENEEY | HLA-A01:01 | POTEE | Colorectal; Uterine | 5291 |
| FPDNESEEY | HLA-A01:01 | POTEE | Colorectal; Uterine | 5292 |
| GLTPLLLGV | HLA-A02:01 | POTEG; POTEE; | Colorectal; Prostate; | 5293 |
| | | POTEH | Uterine | |
| LLLEQNIDV | HLA-A02:01 | POTEG; POTEE; | Colorectal; Prostate; | 5294 |
| | | POTEH | Uterine | |
| GLLENLINGV | HLA-A02:01 | POTEE | Colorectal; Uterine | 5295 |
| KLMAKALLL | HLA-A02:01 | POTEG; POTEE; | Colorectal; Prostate; | 5296 |
| | | POTEH | Uterine | |
| ALAPSMMKI | HLA-A02:01 | POTEE | Colorectal; Uterine | 5297 |
| YLEDIESV | HLA-A02:01 | POTEE | Colorectal; Uterine | 5298 |
| HGLTPLLLGV | HLA-A02:01 | POTEG; POTEE; | Colorectal; Prostate; | 5299 |
| | | POTEH | Uterine | |
| TMDDDTAVL | HLA-A02:01 | POTEE | Colorectal; Uterine | 5300 |
| FLIKKKANLNAL | HLA-A02:01 | POTEG; POTEE; | Colorectal; Prostate; | 5301 |
| | | POTEH | Uterine | |
| ALFQPCFLGM | HLA-A02:01 | POTEE | Colorectal; Uterine | 5302 |
| RMQKEIAAL | HLA-A02:01 | POTEE | Colorectal; Uterine | 5303 |
| LLENLTNGV | HLA-A02:01 | POTEE | Colorectal; Uterine | 5304 |
| TMDDDTAVLV | HLA-A02:01 | POTEE | Colorectal; Uterine | 5305 |
| RELPDYLMKI | HLA-A02:01 | POTEE | Colorectal; Uterine | 5306 |
| LLLDRRCQLNV | HLA-A02:01 | POTEE | Colorectal; Uterine | 5307 |
| KYLEDIESV | HLA-A02:01 | POTEE | Colorectal; Uterine | 5308 |
| TLREELAML | HLA-A02:01 | POTEE | Colorectal; Uterine | 5309 |
| FLIKKKANL | HLA-A02:01 | POTEG; POTEE; | Colorectal; Prostate; | 5310 |
| | | POTEH | Ine | |
| ILIHEEKQIEV | HLA-A02:01 | POTEG; POTEE; | Colorectal; Prostate; | 5311 |
| | | POTEH | Uterine | |
| GLLENLTNG | HLA-A02:01 | POTEE | Colorectal; Uterine | 5312 |
| LLLDRRCQL | HLA-A02:01 | POTEG; POTEE; | Colorectal; Prostate; | 5313 |
| | | POTEH | Uterine | |
| KLMAKALLLY | HLA-A03:01 | POTEG; POTEE; | Colorectal; Prostate; | 5314 |
| | | POTEH | Uterine | |
| TMYPGMAHRMQK | HLA-A03:01 | POTEE | Colorectal; Uterine | 5315 |
| ATYNEDKLMAK | HLA-A03:01 | POTEG; POTEE; | Colorectal; Prostate; | 5316 |
| | | POTEH | Uterine | |
| GMMGGMHQK | HLA-A03:01 | POTEE | Colorectal; Uterine | 5317 |
| KTLRSKMGK | HLA-A03:01 | POTEG; POTEE; | Colorectal; Prostate; | 5318 |
| | | POTEH | Uterine | |
| RIIAPPKRK | HLA-A03:01 | POTEE | Colorectal; Uterine | 5319 |
| ILLTEAPLNPK | HLA-A03:01 | POTEE | Colorectal; Uterine | 5320 |
| ALFQPCFLGM | HLA-A03:01 | POTEE | Colorectal:Uterine | 5321 |
| AALAPSMMK | HLA-ALL:01 | POTEE | Colorectal; Uterine | 5322 |
| SAMKTLRNK | HLA-A11:01 | POTEE | Colorectal; Uterine | 5323 |
| SAMKTLRSK | HLA-A11:01 | POTEG; POTEE; | Colorectal; Prostate; | 5324 |
| | | POTEH | nc | |
| QVVKFLIKK | HLA-A11:01 | POTEG; POTEE; | Colorectal; Prostate; | 5325 |
| | | POTEH | Uterine | |
| GMMGGMHQK | HLA-A11:01 | POTEE | Colorectal; Uterine | 5326 |
| RSQEPEINK | HLA-A11:01 | POTEE | Colorectal; Uterine | 5327 |
| ATYNEDKLMAK | HLA-A11:01 | POTEG; POTEE; | Colorectal; Prostate; | 5328 |
| | | POTEH | Uterine | |
| RYGRTALIL | HLA-A24:02 | POTEG; POTEE; | Colorectal; Prostate; | 5329 |
| | | POTEH | Uterine | |
| MYPGMAHRM | HLA-A24:02 | POTEE | Colorectal; Uterine | 5330 |
| KYLEDIESV | HLA-A24:02 | POTEE | Colorectal; Uterine | 5331 |
| KYPMEHGII | HLA-A24:02 | POTEE | Colorectal; Uterine | 5332 |
| EYHRICELL | HLA-A24:02 | POTEE | Colorectal; Uterine | 5333 |
| ILTERGYRF | HLA-A24:02 | POTEE | Colorectal; Uterine | 5334 |
| KIRIIAPPK | HLA-A30:01 | POTEE | Colorectal; Uterine | 5335 |
| KTLRSKMGK | HLA-A30:01 | POTEG; POTEE; | Colorectal; Prostate; | 5336 |
| | | POTEH | Uterine | |
| KTLRNKMGK | HLA-A30:01 | POTEE | Colorectal; Uterine | 5337 |
| RTALHLASA | HLA-A30:01 | POTEG; POTEE; | Colorectal; Prostate; | 5338 |
| | | POTEH | Uterine | |
| RYGRTALILA | HLA-A30:01 | POTEG; POTEE; | Colorectal; Prostate; | 5339 |
| | | POTEH | Uterine | |
| RSQEPEINK | HLA-A30:01 | POTEE | Colorectal; Uterine | 5340 |
| VVKFLIKKK | HLA-A30:01 | POTEG; POTEE; | Colorectal; Prostate; | 5341 |
| | | POTEH | Uterine | |
| HQKESYVGK | HLA-A30:01 | POTEE | Colorectal; Uterine | 5342 |
| MLRDTDVNK | HLA-A30:01 | POTEE | Colorectal; Uterine | 5343 |
| SVKKPFGLR | HLA-A33:03 | POTEG; POTEE; | Colorectal; Prostate; | 5344 |
| | | POTEH | Uterine | |
| TMYPGMAHR | HLA-A33:03 | POTEE | Colorectal; Uterine | 5345 |
| TTMYPGMAHR | HLA-A33:03 | POTEE | Colorectal; Uterine | 5346 |
| AAWWGKVPR | HLA-A33:03 | POTEG; POTEE; | Colorectal; Prostate; | 5347 |
| | | POTEH | Uterine | |
| NALPHATLR | HLA-A33:03 | POTEE | Colorectal; Uterine | 5348 |
| NSNPENVSR | HLA-A33:03 | POTEE | Colorectal; Uterine | 5349 |
| EVVKLLLDR | HLA-A33:03 | POTEG; POTEE; | Colorectal; Prostate; | 5350 |
| | | POTEH | Uterine | |
| ENSNPENVSR | HLA-A33:03 | POTEE | Colorectal; Uterine | 5351 |
| KPFGLRSKM | HLA-B07:02 | POTEG; POTEE; | Colorectal; Prostate; | 5352 |
| | | POTEH | Uterine | |
| VPRKDLIVM | HLA-B07:02 | POTEG; POTEE; | Colorectal; Prostate; | 5353 |
| | | POTEH | Uterine | |
| RPRQQGMM | HLA-B07:02 | POTEE | Colorectal; Uterine | 5354 |
| VPIYEGNAL | HLA-B07:02 | POTEE | Colorectal; Uterine | 5355 |
| RPRQQGMMGGM | HLA-B07:02 | POTEE | Colorectal; Uterine | 5356 |
| IPDEYGNTTL | HLA-B07:02 | POTEE | Colorectal; Uterine | 5357 |
| APEEHPILL | HLA-B07:02 | POTEE | Colorectal; Uterine | 5358 |
| VPRKDLIVML | HLA-B07:02 | POTEG; POTEE; | Colorectal; Prostate; | 5359 |
| | | POTEH | Uterine | |
| FLIKKKANL | HLA-B08:01 | POTEG; POTEE; | Colorectal; Prostate; | 5360 |
| | | POTEH | Uterine | |
| LLLDRRCQL | HLA-B08:01 | POTEG; POTEE; | Colorectal; Prostate; | 5361 |
| | | POTEH | Uterine | |
| MMKIRITAP | HLA-B08:01 | POTEE | Colorectal; Uterine | 5362 |
| VLDNKKRTAL | HLA-B08:01 | POTEE | Colorectal; Uterine | 5363 |
| FLIKKKANLNAL | HLA-B08:01 | POTEG; POTEE; | Colorectal; Prostate; | 5364 |
| | POTEH | Uterine | | |
| MMKIRILA | HLA-B08:01 | POTEE | Colorectal; Uterine | 5365 |
| APPKRKYSV | HLA-B08:01 | POTEE | Colorectal; Uterine | 5366 |
| DYKEKQML | HLA-B08:01 | POTEG; POTEE | Colorectal; Prostate; | 5367 |
| | | | Uterine | |
| ESKNKHGL | HLA-B08:01 | POTEG; POTEE; | Colorectal; Prostate; | 5368 |
| | | POTEH | Uterine | |
| EEYHRICEL | HLA-B08:01 | POTEE | Colorectal; Uterine | 5369 |
| RELENFMAI | HLA-B13:02 | POTEE | Colorectal; Uterine | 5370 |
| RELPDYLMKI | HLA-B13:02 | POTEE | Colorectal; Uterine | 5371 |
| KQQVVKFLI | HLA-B13:02 | POTEG; POTEE; | Colorectal; Prostate; | 5372 |
| | | POTEH | Uterine | |
| REYAVSSHHHVI | HLA-B13:02 | POTEE | Colorectal; Uterine | 5373 |
| DRELENFMAI | HLA-B13:02 | POTEE | Colorectal; Uterine | 5374 |
| ALAPSMMKI | HLA-B13:02 | POTEE | Colorectal; Uterine | 5375 |
| ELPDYLMKI | HLA-B13:02 | POTEE | Colorectal; Uterine | 5376 |
| GLTPLLLGV | HLA-B13:02 | POTEG; POTEE; | Colorectal; Prostate; | 5377 |
| | | POTEH | Uterine | |
| KQIEVVEKM | HLA-B13:02 | POTEE | Colorectal; Uterine | 5378 |
| YSVWVGGSI | HLA-B46:01 | POTEE | Colorectal; Uterine | 5379 |
| LMAKALLLY | HLA-B46:01 | POTEG; POTEE; | Colorectal; Prostate; | 5380 |
| | | POTEH | Uterine | |
| IAALAPSMM | HLA-B46:01 | POTEE | Colorectal; Uterine | 5381 |
| YAIYNEDKL | HLA-B46:01 | POTEG; POTEE; | Colorectal; Prostate; | 5382 |
| | | POTEH | Uterine | |
| VAIQAVPSL | HLA-B46:01 | POTEE | Colorectal; Uterine | 5383 |
| IIAPPKRKY | HLA-B46:01 | POTEE | Colorectal; Uterine | 5384 |
| TAREYAVSS | HLA-B46:01 | POTEG; POTEE; | Colorectal; Prostate; | 5385 |
| | | POTEH | Uterine | |
| INKDGDREL | HLA-B46:01 | POTEE | Colorectal; Uterine | 5386 |
| TMYPGMAH | HLA-B46:01 | POTEE | Colorectal; Uterine | 5387 |
| SQRFKGSEN | HLA-B46:01 | POTEE | Colorectal; Uterine | 5388 |
| TMDDDTAVL | HLA-C01:02 | POTEE | Colorectal; Uterine | 5389 |
| ALPHATLRL | HLA-C01:02 | POTEE | Colorectal; Uterine | 5390 |
| RMQKEIAAL | HLA-C01:02 | POTEE | Colorectal; Uterine | 5391 |
| VAIQAVPSL | HLA-C01:02 | POTEE | Colorectal; Uterine | 5392 |
| KLMAKALLL | HLA-C01:02 | POTEG; POTEE; | Colorectal; Prostate; | 5393 |
| | | POTEH | | |
| TVPIYEGNAL | HLA-C01:02 | POTEE | Colorectal; Uterine | 5394 |
| VAPEEHPIL | HLA-C01:02 | POTEE | Colorectal; Uterine | 5395 |
| NIPDEYGNTIL | HLA-C01:02 | POTEE | Colorectal; Uterine | 5396 |
| SQPEKMSQEL | HLA-C01:02 | POTEE | Colorectal; Uterine | 5397 |
| VAIQAVPSL | HLA-C03:04 | POTEE | Colorectal; Uterine | 5398 |
| IAALAPSMM | HLA-C03:04 | POTEE | Colorectal; Uterine | 5399 |
| YAVSSHHHV | HLA-C03:04 | POTEE | Colorectal; Uterine | 5400 |
| YSVWVGGSI | HLA-C03:04 | POTEE | Colorectal; Uterine | 5401 |
| YAIYNEDKL | HLA-C03:04 | POTEG; POTEE; | Colorectal; Prostate; | 5402 |
| | | POTEH | Uterine | |
| IAMLRLEL | HLA-C03:04 | POTEE | Colorectal; Uterine | 5403 |
| NALPHATL | HLA-C03:04 | POTEE | Colorectal; Uterine | 5404 |
| TMDDDTAVL | HLA-C04:01 | POTEE | Colorectal; Uterine | 5405 |
| MYPGMAHRM | HLA-C04:01 | POTEE | Colorectal; Uterine | 5406 |
| RLDLAGREL | HLA-C04:01 | POTEE | Colorectal; Uterine | 5407 |
| LFQPCFLGM | HLA-C04:01 | POTEE | Colorectal; Uterine | 5408 |
| WGDYDDSAF | HLA-C04:01 | POTEG; POTEE; | Colorectal; Prostate; | 5409 |
| | | POTEH | Uterine | |
| DYDDSAFM | HLA-C04:01 | POTEG; POTEE; | Colorectal; Prostate; | 5410 |
| | | POTEH | Uterine | |
| MDDDTAVLV | HLA-C04:01 | POTEE | Colorectal; Uterine | 5411 |
| DRYGRTALI | HLA-C07:01 | POTEG; POTEE; | Colorectal; Prostate; | 5412 |
| | | POTEH | Uterine | |
| LDRYGRTALI | HLA-C07:01 | POTEG; POTEE; | Colorectal; Prostate; | 5413 |
| | | POTEH | Uterine | |
| HRAAWWGKV | HLA-C07:01 | POTEG; POTEE; | Colorectal; Prostate; | 5414 |
| | | POTEH | Uterine | |
| HRMQKEIAAL | HLA-C07:01 | POTEE | Colorectal; Uterine | 5415 |
| MYPGMAHRM | HLA-C07:01 | POTEE | Colorectal; Uterine | 5416 |
| DRYGRTALIL | HLA-C07:01 | POTEG; POTEE; | Colorectal; Prostate; | 5417 |
| | | POTEH | Uterine | |
| PRKDLIVML | HLA-C07:01 | POTEG; POTEE; | Uterine | 5418 |
| | | POTEH | Colorectal; Prostate; | |
| RRCQLNVL | HLA-C07:01 | POTEE | Colorectal; Uterine | 5419 |
| SRTPESQQF | HLA-C07:01 | POTEG; POTEE; | Colorectal; Prostate; | 5420 |
| | | POTEH | Uterine | |
| RCFPCYRES | HLA-C07:01 | POTEE | Colorectal; Uterine | 5421 |
| LSGQTAREY | HLA-C07:01 | POTEG; POTEE; | Uterine | 5422 |
| | | POTEH | Colorectal; Prostate; | |
| MYPGMAHRM | HLA-C07:02 | POTEE | Colorectal; Uterine | 5423 |
| DRYGRTALI | HLA-C07:02 | POTEG; POTEE; | Colorectal; Prostate; | 5424 |
| | | POTEH | Uterine | |
| HRMQKEIAAL | HLA-C07:02 | POTEE | Colorectal; Uterine | 5425 |
| RYGRTALIL | HLA-C07:02 | POTEG; POTEE; | Colorectal; Prostate; | 5426 |
| | | POTEH | Uterine | |
| HHHVICQLL | HLA-C07:02 | POTEE | Colorectal; Uterine | 5427 |
| PRKDLIVML | HLA-C07:02 | POTEG; POTEE; | Colorectal; Prostate; | 5428 |
| | | POTEH | Uterine | |
| SRTPESQQF | HLA-C07:02 | POTEG; POTEE; | Colorectal; Prostate; | 5429 |
| | | POTEH | Uterine | |
| VAPEEHPIL | HLA-C07:02 | POTEE | Colorectal; Uterine | 5430 |
| VRGEDLDKL | HLA-C07:02 | POTEE | Colorectal; Uterine | 5431 |
| NTALHYAIY | HLA-A01:01 | POTEG; POTEH | Prostate | 5432 |
| YGNTALHYAIY | HLA-A01:01 | POTEG; POTEH | Prostate | 5433 |
| GNTALHYATY | HLA-A01:01 | POTEG; POTEH | Prostate | 5434 |
| EYGNTALHYAIY | HLA-A01:01 | POTEG; POTEH | Prostate | 5435 |
| FPDTENEQY | HLA-A01:01 | POTEG; POTEH | Prostate | 5436 |
| YGNTALHYAIYN | HLA-A01:01 | POTEG; POTEH | Prostate | 5437 |
| IPDEYGNTALHY | HLA-A01:01 | POTEG; POTEH | Prostate | 5438 |
| LSDYKEKQMILKV | HLA-A01:01 | POTEG | Prostate | 5439 |
| NVICQLLSDY | HLA-A01:01 | POTEG; POTEH | Prostate | 5440 |
| LSEEQNTGIL | HLA-A01:01 | POTEG; POTEH | Prostate | 5441 |
| LSEEQNTGI | HLA-A01:01 | POTEG; POTEH | Prostate | 5442 |
| VLQPQPQLF | HLA-A01:01 | POTEG; POTEH | Prostate | 5443 |
| LSEEQNTG | HLA-A01:01 | POTEG; POTEH | Prostate | 5444 |
| LSEEQNTGILQ | HLA-A01:01 | POTEG; POTEH | Prostate | 5445 |
| TADNGDDGL | HLA-A01:01 | POTEG; POTEH | Prostate | 5446 |
| QLFFSFFLFI | HLA-A02:01 | POTEG; POTEH | Prostate | 5447 |
| QPQLFFSFFL | HLA-A02:01 | POTEG; POTEH | Prostate | 5448 |
| QLFPSFFL | HLA-A02:01 | POTEG; POTEH | Prostate | 5449 |
| FLFIFIFIFI | HLA-A02:01 | POTEG; POTEH | Prostate | 5450 |
| LLLDRRCQLNI | HLA-A02:01 | POTEG; POTEH | Prostate | 5451 |
| PQLFFSFFL | HLA-A02:01 | POTEG; POTEH | Prostate | 5452 |
| QLFFSFFLFIFI | HLA-A02:01 | POTEG; POTEH | Prostate | 5453 |
| FSFFLFIFI | HLA-A02:01 | POTEG; POTEH | Prostate | 5454 |
| QLFFSFFLF | HLA-A02:01 | POTEG; POTEH | Prostate | 5455 |
| FLFIFIFI | HLA-A02:01 | POTEG; POTEH | Prostate | 5456 |
| SLQPRPPEA | HLA-A02:01 | POTEG; POTEH | Prostate | 5457 |
| LLDRRCQLNI | HLA-A02:01 | POTEG; POTEH | Prostate | 5458 |
| KLLLDRRCQLNI | HLA-A02:01 | POTEG; POTEH | Prostate | 5459 |
| MVAEAGSMPA | HLA-A02:01 | POTEG; POTEH | Prostate | 5460 |
| GSMPAASSV | HLA-A02:01 | POTEG; POTEH | Prostate | 5461 |
| PQLFFSFFLFI | HLA-A02:01 | POTEG; POTEH | Prostate | 5462 |
| LLLDRRCQLNIL | HLA-A02:01 | POTEG; POTEH | Prostate | 5463 |
| GILQDEILI | HLA-A02:01 | POTEG; POTEH | Prostate | 5464 |
| QPQPQLFFSFFL | HLA-A02:01 | POTEG; POTEH | Prostate | 5465 |
| YAVSSHHNV | HLA-A02:01 | POTEG | Prostate | 5466 |
| ILQDEILI | HLA-A02:01 | POTEG; POTEH | Prostate | 5467 |
| QPQLFFSFFLFI | HLA-A02:01 | POTEG; POTEH | Prostate | 5468 |
| FLFIFIFIF | HLA-A02:01 | POTEG; POTEH | Prostate | 5469 |
| FFLFIFIFIFI | HLA-A02:01 | POTEG; POTEH | Prostate | 5470 |
| FFSFFLFIFI | HLA-A02:01 | POTEG; POTEH | Prostate | 5471 |
| LFFSFFLFI | HLA-A02:01 | POTEG; POTEH | Prostate | 5472 |
| SFFLFIFIFIFI | HLA-A02:01 | POTEG; POTEH | Prostate | 5473 |
| VLQPQPQL | HLA-A02:01 | POTEG; POTEH | Prostate | 5474 |
| ILQDEILIH | HLA-A02:01 | POTEG; POTEH | Prostate | 5475 |
| ILDNKKRTA | HLA-A02:01 | POTEG; POTEH | Prostate | 5476 |
| ILDNKKRTAL | HLA-A02:01 | POTEG; POTEH | Prostate | 5477 |
| VLQPQPQLF | HLA-A02:01 | POTEG; POTEH | Prostate | 5478 |
| ILQDEILIHE | HLA-A02:01 | POTEG; POTEH | Prostate | 5479 |
| QLSEEQNTGI | HLA-A02:01 | POTEG; POTEH | Prostate | 5480 |
| MVLQPQPQL | HLA-A02:01 | POTEG; POTEH | Prostate | 5481 |
| THMGFPENL | HLA-A02:01 | POTEG; POTEH | Prostate | 5482 |
| ILQDEILIHEE | HLA-A02:01 | POTEG; POTEH | Prostate | 5483 |
| SLQPRPPEAQ | HLA-A02:01 | POTEG; POTEH | Prostate | 5484 |
| VLQPQPQLFF | HLA-A02:01 | POTEG; POTEH | Prostate | 5485 |
| QLSEEQNTG | HLA-A02:01 | POTEG; POTEH | Prostate | 5486 |
| GHPNFPTTL | HLA-A02:01 | POTEG | Prostate | 5487 |
| IPDEYGNTAL | HLA-A02:01 | POTEG; POTEH | Prostate | 5488 |
| ALHYAIYNE | HLA-A02:01 | POTEG; POTEH | Prostate | 5489 |
| FFLFIFIFI | HLA-A02:01 | POTEG; POTEH | Prostate | 5490 |
| IPDEYGNTA | HLA-A02:01 | POTEG; POTEH | Prostate | 5491 |
| VMLKDTDMNK | HLA-A03:01 | POTEG; POTEH | Prostate | 5492 |
| GSMPAASSVK | HLA-A03:01 | POTEG; POTEH | Prostate | 5493 |
| MLKDTDMNK | HLA-A03:01 | POTEG; POTEH | Prostate | 5494 |
| MLKDTDMNKK | HLA-A03:01 | POTEG; POTEH | Prostate | 5495 |
| KVPRKDLIVMLK | HLA-A03:01 | POTEG; POTEH | Prostate | 5496 |
| LDNKKRTALTK | HLA-A03:01 | POTEG; POTEH | Prostate | 5497 |
| ALHYAIYNEDK | HLA-A03:01 | POTEG; POTEH | Prostate | 5498 |
| GSMPAASSVKK | HLA-A03:01 | POTEG; POTEH | Prostate | 5499 |
| IVMLKDTDMNK | HLA-A03:01 | POTEG; POTEH | Prostate | 5500 |
| ILQDEILIH | HLA-A03:01 | POTEG; POTEH | Prostate | 5501 |
| HCFPWCRGSGK | HLA-A03:01 | POTEG; POTEH | Prostate | 5502 |
| GILQDEILIH | HLA-A03:01 | POTEG; POTEH | Prostate | 5503 |
| QLFFSFFLF | HLA-A03:01 | POTEG; POTEH | Prostate | 5504 |
| FLFIFIFIFI | HLA-A03:01 | POTEG; POTEH | Prostate | 5505 |
| VLQPQPQLFF | HLA-A03:01 | POTEG; POTEH | Prostate | 5506 |
| FIPIFIFIFF | HLA-A03:01 | POTEG; POTEH | Prostate | 5507 |
| FLFIFIFIF | HLA-A03:01 | POTEG; POTEH | Prostate | 5508 |
| SVSQAGVQW | HLA-A03:01 | POTEG; POTEH | Prostate | 5509 |
| GSMPAASSVK | HLA-A11:01 | POTEG; POTEH | Prostate | 5510 |
| MSQEPEINK | HLA-A11:01 | POTEG; POTEH | Prostate | 5511 |
| GSMPAASSVKK | HLA-A11:01 | POTEG; POTEH | Prostate | 5512 |
| VMLKDTDMNK | HLA-A11:01 | POTEG; POTEH | Prostate | 5513 |
| KVPRKDLIVMLK | HLA-A11:01 | POTEG; POTEH | Prostate | 5514 |
| TALHYAIYNEDK | HLA-A11:01 | POTEG; POTEH | Prostate | 5515 |
| CQLNILDNK | HLA-A11:01 | POTEG; POTEH | Prostate | 5516 |
| MLKDTDMNK | HLA-A11:01 | POTEG; POTEH | Prostate | 5517 |
| IVMLKDTDMNK | HLA-AII:01 | POTEG; POTEH | Prostate | 5518 |
| SVSQAGVQW | HLA-A11:01 | POTEG; POTEH | Prostate | 5519 |
| RKVEEEMKK | HLA-A11:01 | POTEG; POTEH | Prostate | 5520 |
| RKDLIVMLK | HLA-A11:01 | POTEG; POTEH | Prostate | 5521 |
| GILQDEILIH | HLA-A11:01 | POTEG; POTEH | Prostate | 5522 |
| FSFFLFIFI | HLA-A11:01 | POTEG; POTEH | Prostate | 5523 |
| PQPQLFFSF | HLA-A24:02 | POTEG; POTEH | Prostate | 5524 |
| KWCRHCFPW | HLA-A24:02 | POTEG | Prostate | 5525 |
| QLFFSFFLF | HLA-A24:02 | POTEG; POTEH | Prostate | 5526 |
| LFFSFFLF | HLA-A24:02 | POTEG; POTEH | Prostate | 5527 |
| PQLFFSFFLF | HLA-A24:02 | POTEG; POTEH | Prostate | 5528 |
| QPQPQLFFSF | HLA-A24:02 | POTEG; POTEH | Prostate | 5529 |
| VLQPQPQLF | HLA-A24:02 | POTEG; POTEH | Prostate | 5530 |
| VLQPQPQLFFSF | HLA-A24:02 | POTEG; POTEH | Prostate | 5531 |
| SFFLFIFIF | HLA-A24:02 | POTEG; POTEH | Prostate | 5532 |
| PQPQLFFSFF | HLA-A24:02 | POTEG; POTEH | Prostate | 5533 |
| IFIFIFIFF | HLA-A24:02 | POTEG; POTEH | Prostate | 5534 |
| FFSFFLFIF | HLA-A24:02 | POTEG; POTEH | Prostate | 5535 |
| LFIFIFIFI | HLA-A24:02 | POTEG; POTEH | Prostate | 5536 |
| VLQPQPQLFF | HLA-A24:02 | POTEG; POTEH | Prostate | 5537 |
| GSMPAASSVK | HLA-A30:01 | POTEG; POTEH | Prostate | 5538 |
| HVRREDLDK | HLA-A30:01 | POTEG; POTEH | Prostate | 5539 |
| MLKDTDMNK | HLA-A30:01 | POTEG; POTEH | Prostate | 5540 |
| GSMPAASSV | HLA-A30:01 | POTEG; POTEH | Prostate | 5541 |
| SSKSNVGTS | HLA-A30:01 | POTEG | Prostate | 5542 |
| STHMGFPEN | HLA-A30:01 | POTEG; POTEH | Prostate | 5543 |
| STHMGFPENL | HLA-A30:01 | POTEG; POTEH | Prostate | 5544 |
| KNKIESHSV | HLA-A30:01 | POTEG; POTEH | Prostate | 5545 |
| RYHVRREDLDK | HLA-A30:01 | POTEG; POTEH | Prostate | 5546 |
| RSKMGKWCR | HLA-A30:01 | POTEG; POTEH | Prostate | 5547 |
| MSQEPEINK | HLA-A30:01 | POTEG; POTEH | Prostate | 5548 |
| MVLQPQPQL | HLA-A30:01 | POTEG; POTEH | Prostate | 5549 |
| CQLNILDNK | HLA-A30:01 | POTEG; POTEH | Prostate | 5550 |
| KSKVGPWGD | HLA-A30:01 | POTEG | Prostate | 5551 |
| RDFSGHPNF | HLA-A30:01 | POTEG | Prostate | 5552 |
| RKVEEEMKK | HLA-A30:01 | POTEG; POTEH | Prostate | 5553 |
| FMEPRYHVRR | HLA-A33:03 | POTEG; POTEH | Prostate | 5554 |
| RSKMGKWCR | HLA-A33:03 | POTEG; POTEH | Prostate | 5555 |
| AFMEPRYHVRR | HLA-A33:03 | POTEG; POTEH | Prostate | 5556 |
| SAFMEPRYHVRR | HLA-A33:03 | POTEG; POTEH | Prostate | 5557 |
| TLRSKMGKWCR | HLA-A33:03 | POTEG | Prostate | 5558 |
| CFPWCRGSSK | HLA-A33:03 | POTEG | Prostate | 5559 |
| WCRHCFPWCR | HLA-A33:03 | POTEG | Prostate | 5560 |
| LRSKMGKWCR | HLA-A33:03 | POTEG; POTEH | Prostate | 5561 |
| CFPWCRGSGK | HLA-A33:03 | POTEG; POTEH | Prostate | 5562 |
| DTENEQYHR | HLA-A33:03 | POTEG | Prostate | 5563 |
| NILDNKKR | HLA-A33:03 | POTEG; POTEH | Prostate | 5564 |
| TALTKAVQCR | HLA-A33:03 | POTEG | Prostate | 5565 |
| MNKKDKQKR | HLA-A33:03 | POTEG; POTEH | Prostate | 5566 |
| TALTKAVQC | HLA-A33:03 | POTEG; POTEH | Prostate | 5567 |
| EPRYHVRR | HLA-A33:03 | POTEG; POTEH | Prostate | 5568 |
| MSQEPEINK | HLA-A33:03 | POTEG; POTEH | Prostate | 5569 |
| EINKGGDR | HLA-A33:03 | POTEG; POTEH | Prostate | 5570 |
| DMNKKDKQKR | HLA-A33:03 | POTEG; POTEH | Prostate | 5571 |
| HPNFPTTLPI | HLA-B07:02 | POTEG | Prostate | 5572 |
| QPRPPEAQM | HLA-B07:02 | POTEG; POTEH | Prostate | 5573 |
| LQPRPPEAQM | HLA-B07:02 | POTEG; POTEH | Prostate | 5574 |
| RPPEAQMVL | HLA-B07:02 | POTEG; POTEH | Prostate | 5575 |
| IPDEYGNTAL | HLA-B07:02 | POTEG; POTEH | Prostate | 5576 |
| QPRPPEAQMVL | HLA-B07:02 | POTEG; POTEH | Prostate | 5577 |
| HPNFPTTL | HLA-B07:02 | POTEG | Prostate | 5578 |
| SLQPRPPEAQM | HLA-B07:02 | POTEG; POTEH | Prostate | 5579 |
| SSLQPRPPEAQM | HLA-B07:02 | POTEG; POTEH | Prostate | 5580 |
| FPWCRGSSKSNV | HLA-B07:02 | POTEG | Prostate | 5581 |
| QPRPPEAQMV | HLA-B07:02 | POTEG; POTEH | Prostate | 5582 |
| QPQLFFSFF | HLA-B07:02 | POTEG; POTEH | Prostate | 5583 |
| QPQPQLFESP | HLA-B07:02 | POTEG; POTEH | Prostate | 5584 |
| QPQPQLFF | HLA-B07:02 | POTEG; POTEH | Prostate | 5585 |
| ILDNKKRTAL | HLA-B08:01 | POTEG; POTEH | Prostate | 5586 |
| MNKKDKQKRTAL | HLA-B08:01 | POTEG; POTEH | Prostate | 5587 |
| EMKKHGSTHM | HLA-B08:01 | POTEG; POTEH | Prostate | 5588 |
| NILDNKKRTAL | HLA-B08:01 | POTEG; POTEH | Prostate | 5589 |
| LLLDRRCQLNIL | HLA-B08:01 | POTEG; POTEH | Prostate | 5590 |
| LNILDNKKRTAL | HLA-B08:01 | POTEG; POTEH | Prostate | 5591 |
| FPWCRGSSKSNV | HLA-B08:01 | POTEG | Prostate | 5592 |
| MGKWCRHCF | HLA-B08:01 | POTEG; POTEH | Prostate | 5593 |
| DRRCQLNIL | HLA-B08:01 | POTEG; POTEH | Prostate | 5594 |
| HPNFPTTL | HLA-B08:01 | POTEG | Prostate | 5595 |
| ESKNKIESH | HLA-B08:01 | POTEG; POTEH | Prostate | 5596 |
| FIFIFIFF | HLA-B08:01 | POTEG; POTEH | Prostate | 5597 |
| EEKQIEVA | HLA-B08:01 | POTEG; POTEH | Prostate | 5598 |
| TALTKAVQ | HLA-B08:01 | POTEG; POTEH | Prostate | 5599 |
| DEYGNTAL | HLA-B08:01 | POTEG; POTEH | Prostate | 5600 |
| FLFIFIFIF | HLA-B08:01 | POTEG; POTEH | Prostate | 5601 |
| QPQLFFSF | HLA-B08:01 | POTEG; POTEH | Prostate | 5602 |
| HHNVICQL | HLA-B08:01 | POTEG | Prostate | 5603 |
| HPNFPTTLPI | HLA-B13:02 | POTEG | Prostate | 5604 |
| REYAVSSHHNVI | HLA-B13:02 | POTEG | Prostate | 5605 |
| REDECALMLL | HLA-B13:02 | POTEG | Prostate | 5606 |
| FSFFLFIFI | HLA-B13:02 | POTEG; POTEH | Prostate | 5607 |
| QPQLFFSFFL | HLA-B13:02 | POTEG; POTEH | Prostate | 5608 |
| LDRRCQLNI | HLA-B13:02 | POTEG; POTEH | Prostate | 5609 |
| QLFFSFFLFI | HLA-B13:02 | POTEG; POTEH | Prostate | 5610 |
| QLFFSFFLF | HLA-B13:02 | POTEG; POTEH | Prostate | 5611 |
| REYAVSSHHNV | HLA-B13:02 | POTEG | Prostate | 5612 |
| REDECALML | HLA-B13:02 | POTEG | Prostate | 5613 |
| SQEPEINKG | HLA-B13:02 | POTEG; POTEH | Prostate | 5614 |
| KQLSEEQNT | HLA-B13:02 | POTEG; POTEH | Prostate | 5615 |
| SQAGVQWCD | HLA-B13:02 | POTEG; POTEH | Prostate | 5616 |
| KQIEVAENE | HLA-B13:02 | POTEG; POTEH | Prostate | 5617 |
| GILQDEILI | HLA-B13:02 | POTEG; POTEH | Prostate | 5618 |
| ADIESKNKI | HLA-B13:02 | POTEG; POTEH | Prostate | 5619 |
| AVSSHHNVI | HLA-B13:02 | POTEG | Prostate | 5620 |
| EQNDTQKQL | HLA-B13:02 | POTEG; POTEH | Prostate | 5621 |
| RPPEAQMV | HLA-B13:02 | POTEG; POTEH | Prostate | 5622 |
| YAVSSHHNV | HLA-B46:01 | POTEG | Prostate | 5623 |
| GSMPAASSV | HLA-B46:01 | POTEG; POTEH | Prostate | 5624 |
| NTALHYAIY | HLA-B46:01 | POTEG; POTEH | Prostate | 5625 |
| YAVSSHHNVI | HLA-B46:01 | POTEG | Prostate | 5626 |
| MVAEAGSMPA | HLA-B46:01 | POTEG; POTEH | Prostate | 5627 |
| VLQPQPQLF | HLA-B46:01 | POTEG; POTEH | Prostate | 5628 |
| MVLQPQPQLP | HLA-B46:01 | POTEG; POTEH | Prostate | 5629 |
| FSFFLFIFI | HLA-B46:01 | POTEG; POTEH | Prostate | 5630 |
| LQPQPQLFF | HLA-B46:01 | POTEG; POTEH | Prostate | 5631 |
| ESKNKIESH | HLA-B46:01 | POTEG; POTEH | Prostate | 5632 |
| KSKVGPWGDY | HLA-B46:01 | POTEG | Prostate | 5633 |
| FIFIFIFIF | HLA-B46:01 | POTEG; POTEH | Prostate | 5634 |
| FLFIFIFIF | HLA-B46:01 | POTEG; POTEH | Prostate | 5635 |
| KSKVGPWGD | HLA-B46:01 | POTEG | Prostate | 5636 |
| SLQPRPPEA | HLA-B46:01 | POTEG; POTEH | Prostate | 5637 |
| MLKVSSEN | HLA-B46:01 | POTEG | Prostate | 5638 |
| YHRDFSGH | HLA-B46:01 | POTEG | Prostate | 5639 |
| RPPEAQMVL | HLA-C01:02 | POTEG; POTEH | Prostate | 5640 |
| GSMPAASSV | HLA-C01:02 | POTEG; POTEH | Prostate | 5641 |
| VLQPQPQLF | HLA-C01:02 | POTEG; POTEH | Prostate | 5642 |
| FSGHPNFPTTL | HLA-C01:02 | POTEG | Prostate | 5643 |
| YAVSSHHNV | HLA-C01:02 | POTEG | Prostate | 5644 |
| MVLQPQPQL | HLA-C01:02 | POTEG; POTEH | Prostate | 5645 |
| AVSSHHNVI | HLA-C01:02 | POTEG | Prostate | 5646 |
| LQPQPQLFF | HLA-C01:02 | POTEG; POTEH | Prostate | 5647 |
| SGHPNFPTTL | HLA-C01:02 | POTEG | Prostate | 5648 |
| NLPNGATAD | HLA-C01:02 | POTEG | Prostate | 5649 |
| NIPDEYGNTAL | HLA-C01:02 | POTEG; POTEH | Prostate | 5650 |
| GHPNFPTTL | HLA-C01:02 | POTEG | Prostate | 5651 |
| NLPNGATA | HLA-C01:02 | POTEG | Prostate | 5652 |
| NFPTTLPI | HLA-C01:02 | POTEG | Prostate | 5653 |
| NLPNGATADN | HLA-C01:02 | POTEG | Prostate | 5654 |
| RPPEAQMV | HLA-C01:02 | POTEG; POTEH | Prostate | 5655 |
| YAVSSHHNV | HLA-C03:04 | POTEG | Prostate | 5656 |
| GSMPAASSV | HLA-C03:04 | POTEG; POTEH | Prostate | 5657 |
| YAVSSHHNVI | HLA-C03:04 | POTEG | Prostate | 5658 |
| MVLQPQPQL | HLA-C03:04 | POTEG; POTEH | Prostate | 5659 |
| AVSSHHNVI | HLA-C03:04 | POTEG | Prostate | 5660 |
| IVMLKDTDM | HLA-C03:04 | POTEG; POTEH | Prostate | 5661 |
| AGSMPAASSV | HLA-C03:04 | POTEG; POTEH | Prostate | 5662 |
| FSFFLFIFI | HLA-C03:04 | POTEG; POTEH | Prostate | 5663 |
| RPPEAQMVL | HLA-C03:04 | POTEG; POTEH | Prostate | 5664 |
| FSGHPNFPTTL | HLA-C03:04 | POTEG | Prostate | 5665 |
| FIFIFIFF | HLA-C03:04 | POTEG; POTEH | Prostate | 5666 |
| FIFIFIFIF | HLA-C03:04 | POTEG; POTEH | Prostate | 5667 |
| FIFIFIFI | HLA-C03:04 | POTEG; POTEH | Prostate | 5668 |
| VLQPQPQLF | HLA-C04:01 | POTEG; POTEH | Prostate | 5669 |
| HHNVICQLL | HLA-C04:01 | POTEG | Prostate | 5670 |
| LQPQPQLFF | HLA-C04:01 | POTEG; POTEH | Prostate | 5671 |
| REDECALML | HLA-C04:01 | POTEG | Prostate | 5672 |
| RPPEAQMVL | HLA-C04:01 | POTEG; POTEH | Prostate | 5673 |
| HRDFSGHPNF | HLA-C04:01 | POTEG | Prostate | 5674 |
| CREDECALM | HLA-C04:01 | POTEG | Prostate | 5675 |
| VLQPQPQLFF | HLA-C04:01 | POTEG; POTEH | Prostate | 5676 |
| REDECALMLL | HLA-C04:01 | POTEG | Prostate | 5677 |
| IFIFIFIFF | HLA-C04:01 | POTEG; POTEH | Prostate | 5678 |
| FFSFFLFIF | HLA-C04:01 | POTEG; POTEH | Prostate | 5679 |
| SFFLFIFIF | HLA-C04:01 | POTEG; POTEH | Prostate | 5680 |
| SFFLFIFI | HLA-C04:01 | POTEG; POTEH | Prostate | 5681 |
| FFSFFLFI | HLA-C04:01 | POTEG; POTEH | Prostate | 5682 |
| IFIFIFIF | HLA-C04:01 | POTEG; POTEH | Prostate | 5683 |
| LFFSFFLF | HLA-C04:01 | POTEG; POTEH | Prostate | 5684 |
| FFLFIFIF | HLA-C04:01 | POTEG; POTEH | Prostate | 5685 |
| CREDECALM | HLA-C07:01 | POTEG | Prostate | 5686 |
| HHNVICQLL | HLA-C07:01 | POTEG | Prostate | 5687 |
| SHHNVICQL | HLA-C07:01 | POTEG | Prostate | 5688 |
| KRTALTKAV | HLA-C07:01 | POTEG; POTEH | Prostate | 5689 |
| HRDFSGHPNF | HLA-C07:01 | POTEG | Prostate | 5690 |
| THMGFPENL | HLA-C07:01 | POTEG; POTEH | Prostate | 5691 |
| DRRCQLNIL | HLA-C07:01 | POTEG; POTEH | Prostate | 5692 |
| VRREDLDKL | HLA-C07:01 | POTEG; POTEH | Prostate | 5693 |
| SHHNVICQLL | HLA-C07:01 | POTEG | Prostate | 5694 |
| PRPPEAQMVL | HLA-C07:01 | POTEG; POTEH | Prostate | 5695 |
| RPPEAQMVL | HLA-C07:01 | POTEG; POTEH | Prostate | 5696 |
| PRYHVRRED | HLA-C07:01 | POTEG; POTEH | Prostate | 5697 |
| HPNFPTTL | HLA-C07:01 | POTEG | Prostate | 5698 |
| LPNGATAD | HLA-C07:01 | POTEG | Prostate | 5699 |
| RRCQLNIL | HLA-C07:01 | POTEG; POTEH | Prostate | 5700 |
| RYHVRRED | HLA-C07:01 | POTEG; POTEH | Prostate | 5701 |
| TENEQYHSD | HLA-C07:01 | POTEG; POTEH | Prostate | 5702 |
| REDECALML | HLA-C07:01 | POTEG | Prostate | 5703 |
| SHHNVICQL | HLA-C07:02 | POTEG | Prostate | 5704 |
| HHNVICQLL | HLA-C07:02 | POTEG | Prostate | 5705 |
| GHPNFPTTL | HLA-C07:02 | POTEG | Prostate | 5706 |
| THMGFPENL | HLA-C07:02 | POTEG; POTEH | Prostate | 5707 |
| RYHVRREDL | HLA-C07:02 | POTEG; POTEH | Prostate | 5708 |
| CREDECALM | HLA-C07:02 | POTEG | Prostate | 5709 |
| VLQPQPQLF | HLA-C07:02 | POTEG; POTEH | Prostate | 5710 |
| KRTALTKAV | HLA-C07:02 | POTEG; POTEH | Prostate | 5711 |
| HRDFSGHPNF | HLA-C07:02 | POTEG | Prostate | 5712 |
| PRPPEAQMVL | HLA-C07:02 | POTEG; POTEH | Prostate | 5713 |
| VRREDLDKL | HLA-C07:02 | POTEG; POTEH | Prostate | 5714 |
| PRPPEAQM | HLA-C07:02 | POTEG; POTEH | Prostate | 5715 |
| EYGNTALHY | HLA-C07:02 | POTEG; POTEH | Prostate | 5716 |
| RPPEAQMVL | HLA-C07:02 | POTEG; POTEH | Prostate | 5717 |
| FIFIFIFIF | HLA-C07:02 | POTEG; POTEH | Prostate | 5718 |
| RREDLDKL | HLA-C07:02 | POTEG; POTEH | Prostate | 5719 |
| FLFIFIFIF | HLA-C07:02 | POTEG; POTEH | Prostate | 5720 |
| LSDYKEKQILKV | HLA-A01:01 | POTEH | Prostate | 5721 |
| LSDYKEKQI | HLA-A01:01 | POTEH | Prostate | 5722 |
| LSDYKEKQILK | HLA-A01:01 | POTEH | Prostate | 5723 |
| LSDYKEKQIL | HLA-A01:01 | POTEH | Prostate | 5724 |
| RHNVICQLLSDY | HLA-A01:01 | POTEH | Prostate | 5725 |
| LLSDYKEKQILK | HLA-A01:01 | POTEH | Prostate | 5726 |
| LLSDYKEKQI | HLA-A01:01 | POTEH | Prostate | 5727 |
| VSSRHNVICQLL | HLA-A01:01 | POTEH | Prostate | 5728 |
| MGKWCRHCFA | HLA-A01:01 | POTEH | Prostate | 5729 |
| FAWCRGSGK | HLA-A01:01 | POTEH | Prostate | 5730 |
| YKEKQILKV | HLA-A01:01 | POTEH | Prostate | 5731 |
| YKEKQILK | HLA-A01:01 | POTEH | Prostate | 5732 |
| LTNGATADN | HLA-A01:01 | POTEH | Prostate | 5733 |
| LINGATAD | HLA-A01:01 | POTEH | Prostate | 5734 |
| VSSRHNVIC | HLA-A01:01 | POTEH | Prostate | 5735 |
| LLSDYKEKQI | HLA-A02:01 | POTEH | Prostate | 5736 |
| KMGKWCRHCFA | HLA-A02:01 | POTEH | Prostate | 5737 |
| FAWCRGSGKSNV | HLA-A02:01 | POTEH | Prostate | 5738 |
| YAVSSRHNV | HLA-A02:01 | POTEH | Prostate | 5739 |
| SKMGKWCRHCFA | HLA-A02:01 | POTEH | Prostate | 5740 |
| MGKWCRHCFA | HLA-A02:01 | POTEH | Prostate | 5741 |
| LLSDYKEKQIL | HLA-A02:01 | POTEH | Prostate | 5742 |
| KMGKWCRHCFAW | HLA-A02:01 | POTEH | Prostate | 5743 |
| HMGFPENLT | HLA-A02:01 | POTEH | Prostate | 5744 |
| QLLSDYKEKQIL | HLA-A02:01 | POTEH | Prostate | 5745 |
| QLLSDYKEKQI | HLA-A02:01 | POTEH | Prostate | 5746 |
| CQLLSDYKEKQI | HLA-A02:01 | POTEH | Prostate | 5747 |
| FAWCRGSGK | HLA-A02:01 | POTEH | Prostate | 5748 |
| KMGKWCCHCFPW | HLA-A02:01 | POTEH | Prostate | 5749 |
| AVSSRHNVICQL | HLA-A02:01 | POTEH | Prostate | 5750 |
| GKWCRHCFA | HLA-A02:01 | POTEH | Prostate | 5751 |
| AVSSRHNVI | HLA-A02:01 | POTEH | Prostate | 5752 |
| FAWCRGSGKS | HLA-A02:01 | POTEH | Prostate | 5753 |
| HMGFPENLTNGA | HLA-A02:01 | POTEH | Prostate | 5754 |
| THMGFPENLT | HLA-A02:01 | POTEH | Prostate | 5755 |
| YAVSSRHNVI | HLA-A02:01 | POTEH | Prostate | 5756 |
| LLSDYKEKQILK | HLA-A02:01 | POTEH | Prostate | 5757 |
| KWCCHCFPWC | HLA-A02:01 | POTEH | Prostate | 5758 |
| GKWCCHCFPW | HLA-A02:01 | POTEH | Prostate | 5759 |
| HMGFPENLTN | HLA-A02:01 | POTEH | Prostate | 5760 |
| KQILKVSSE | HLA-A02:01 | POTEH | Prostate | 5761 |
| KWCCHCFPW | HLA-A02:01 | POTEH | Prostate | 5762 |
| YKEKQILKV | HLA-A02:01 | POTEH | Prostate | 5763 |
| SRHNVICQL | HLA-A02:01 | POTEH | Prostate | 5764 |
| ALTKAVQCQ | HLA-A02:01 | POTEH | Prostate | 5765 |
| NLTNGATAD | HLA-A02:01 | POTEH | Prostate | 5766 |
| AVSSRHNV | HLA-A02:01 | POTEH | Prostate | 5767 |
| SDYKEKQILKV | HLA-A02:01 | POTEH | Prostate | 5768 |
| SDYKEKQIL | HLA-A02:01 | POTEH | Prostate | 5769 |
| CRQSGKNKV | HLA-A02:01 | POTEH | Prostate | 5770 |
| DYKEKQILKV | HLA-A02:01 | POTEH | Prostate | 5771 |
| ILKVSSENS | HLA-A02:01 | POTEH | Prostate | 5772 |
| MGFPENLTN | HLA-A02:01 | POTEH | Prostate | 5773 |
| ALTKAVQCQE | HLA-A02:01 | POTEH | Prostate | 5774 |
| RHNVICQLL | HLA-A02:01 | POTEH | Prostate | 5775 |
| NLTNGATADN | HLA-A02:01 | POTEH | Prostate | 5776 |
| QILKVSSEN | HLA-A02:01 | POTEH | Prostate | 5777 |
| SRHNVICQLL | HLA-A02:01 | POTEH | Prostate | 5778 |
| KEKQILKV | HLA-A02:01 | POTEH | Prostate | 5779 |
| LSDYKEKQI | HLA-A02:01 | POTEH | Prostate | 5780 |
| FAWCRGSGK | HLA-A03:01 | POTEH | Prostate | 5781 |
| CFAWCRGSGK | HLA-A03:01 | POTEH | Prostate | 5782 |
| LLSDYKEKQILK | HLA-A03:01 | POTEH | Prostate | 5783 |
| RHCFAWCRGSGK | HLA-A03:01 | POTEH | Prostate | 5784 |
| HCPAWCRGSGK | HLA-A03:01 | POTEH | Prostate | 5785 |
| FAWCRGSGKS | HLA-A03:01 | POTEH | Prostate | 5786 |
| LSDYKERQILK | HLA-A03:01 | POTEH | Prostate | 5787 |
| SDYKEKQILK | HLA-A03:01 | POTEH | Prostate | 5788 |
| TAREYAVSSR | HLA-A03:01 | POTEH | Prostate | 5789 |
| REYAVSSRH | HLA-A03:01 | POTEH | Prostate | 5790 |
| DYKEKQILK | HLA-A03:01 | POTEH | Prostate | 5791 |
| AVSSRHNVIC | HLA-A03:01 | POTEH | Prostate | 5792 |
| REYAVSSR | HLA-A03:01 | POTEH | Prostate | 5793 |
| CCRGSGKNK | HLA-A03:01 | POTEH | Prostate | 5794 |
| FAWCRGSGK | HLA-A11:01 | POTEH | Prostate | 5795 |
| LLSDYKEKQILK | HLA-A11:01 | POTEH | Prostate | 5796 |
| CFAWCRGSGK | HLA-A11:01 | POTEH | Prostate | 5797 |
| LSDYKEKQILK | HLA-A11:01 | POTEH | Prostate | 5798 |
| SDYKEKQILK | HLA-A11:01 | POTEH | Prostate | 5799 |
| RHCFAWCRGSGK | HLA-A11:01 | POTEH | Prostate | 5800 |
| QTAREYAVSSR | HLA-A11:01 | POTEH | Prostate | 5801 |
| TAREYAVSSR | HLA-ALI:01 | POTEH | Prostate | 5802 |
| HCFAWCRGSGK | HLA-A11:01 | POTEH | Prostate | 5803 |
| DYKEKQILK | HLA-A11:01 | POTEH | Prostate | 5804 |
| AVSSRHNVI | HLA-A11:01 | POTEH | Prostate | 5805 |
| AVSSRHNVIC | HLA-A11:01 | POTEH | Prostate | 5806 |
| CCRGSGKNK | HLA-A11:01 | POTEH | Prostate | 5807 |
| AREYAVSSR | HLA-A11:01 | POTEH | Prostate | 5808 |
| MGFPENLTN | HLA-A11:01 | POTEH | Prostate | 5809 |
| KWCCHCFPW | HLA-A24:02 | POTEH | Prostate | 5810 |
| KWCRHCFAW | HLA-A24:02 | POTEH | Prostate | 5811 |
| GKWCCHCEPW | HLA-A24:02 | POTEH | Prostate | 5812 |
| KMGKWCCHCFPW | HLA-A24:02 | POTEH | Prostate | 5813 |
| MGKWCCHCFPW | HLA-A24:02 | POTEH | Prostate | 5814 |
| GKWCRHCFAW | HLA-A24:02 | POTEH | Prostate | 5815 |
| RHNVICQLL | HLA-A24:02 | POTEH | Prostate | 5816 |
| MGKWCRHCFAW | HLA-A24:02 | POTEH | Prostate | 5817 |
| KMGKWCRHCFAW | HLA-A24:02 | POTEH | Prostate | 5818 |
| DYKEKQILK | HLA-A24:02 | POTEH | Prostate | 5819 |
| DYKEKQIL | HLA-A24:02 | POTEH | Prostate | 5820 |
| EYAVSSRHN | HLA-A24:02 | POTEH | Prostate | 5821 |
| EYAVSSRHNVI | HLA-A24:02 | POTEH | Prostate | 5822 |
| EYAVSSRHNV | HLA-A24:02 | POTEH | Prostate | 5823 |
| DYKEKQILKV | HLA-A24:02 | POTEH | Prostate | 5824 |
| AVSSRHNVI | HLA-A24:02 | POTEH | Prostate | 5825 |
| MGKWCRHCFA | HLA-A30:01 | POTEH | Prostate | 5826 |
| SSRHNVICQL | HLA-A30:01 | POTEH | Prostate | 5827 |
| SSRHNVICQ | HLA-A30:01 | POTEH | Prostate | 5828 |
| SDYKEKQILK | HLA-A30:01 | POTEH | Prostate | 5829 |
| KMGKWCRHCFA | HLA-A30:01 | POTEH | Prostate | 5830 |
| AVSSRHNVI | HLA-A30:01 | POTEH | Prostate | 5831 |
| RHCFAWCRGSGK | HLA-A30:01 | POTEH | Prostate | 5832 |
| CCRGSGKNK | HLA-A30:01 | POTEH | Prostate | 5833 |
| KWCRHCFAWC | HLA-A30:01 | POTEH | Prostate | 5834 |
| DYKEKQILK | HLA-A30:01 | POTEH | Prostate | 5835 |
| DYKEKQILKV | HLA-A30:01 | POTEH | Prostate | 5836 |
| SRHNVICQL | HLA-A30:01 | POTEH | Prostate | 5837 |
| KEKQILKV | HLA-A30:01 | POTEH | Prostate | 5838 |
| KNKVGPWGD | HLA-A30:01 | POTEH | Prostate | 5839 |
| AVSSRHNV | HLA-A30:01 | POTEH | Prostate | 5840 |
| TAREYAVSSR | HLA-A33:03 | POTEH | Prostate | 5841 |
| CFAWCRGSGK | HLA-A33:03 | POTEH | Prostate | 5842 |
| WCRHCFAWCR | HLA-A33:03 | POTEH | Prostate | 5843 |
| FAWCRGSGK | HLA-A33:03 | POTEH | Prostate | 5844 |
| CCHCFPWCR | HLA-A33:03 | POTEH | Prostate | 5845 |
| DYKEKQILK | HLA-A33:03 | POTEH | Prostate | 5846 |
| WCCHCFPWCR | HLA-A33:03 | POTEH | Prostate | 5847 |
| CRHCFAWCR | HLA-A33:03 | POTEH | Prostate | 5848 |
| QTAREYAVSSR | HLA-A33:03 | POTEH | Prostate | 5849 |
| EYAVSSRHN | HLA-A33:03 | POTEH | Prostate | 5850 |
| EYAVSSRHNV | HLA-A33:03 | POTEH | Prostate | 5851 |
| DYKEKQILKV | HLA-A33:03 | POTEH | Prostate | 5852 |
| EYAVSSRH | HLA-A33:03 | POTEH | Prostate | 5853 |
| ENLTNGATAD | HLA-A33:03 | POTEH | Prostate | 5854 |
| AVSSRHNVI | HLA-B07:02 | POTEH | Prostate | 5855 |
| YAVSSRHNV | HLA-B07:02 | POTEH | Prostate | 5856 |
| YAVSSRHNVI | HLA-B07:02 | POTEH | Prostate | 5857 |
| SSRHNVICQL | HLA-B07:02 | POTEH | Prostate | 5858 |
| FPCCRGSGKNKV | HLA-B07:02 | POTEH | Prostate | 5859 |
| FAWCRGSGK | HLA-B07:02 | POTEH | Prostate | 5860 |
| FPENLTNGATAD | HLA-B07:02 | POTEH | Prostate | 5861 |
| MGFPENLINGAT | HLA-B07:02 | POTEH | Prostate | 5862 |
| REYAVSSRHNVI | HLA-B07:02 | POTEH | Prostate | 5863 |
| RHNVICQLL | HLA-B07:02 | POTEH | Prostate | 5864 |
| SDYKEKQIL | HLA-B07:02 | POTEH | Prostate | 5865 |
| SRHNVICQL | HLA-B07:02 | POTEH | Prostate | 5866 |
| AVSSRHNV | HLA-B07:02 | PQTER | Prostate | 5867 |
| RHNVICQL | HLA-B07:02 | POTEH | Prostate | 5868 |
| FPCCRGSGKN | HLA-B07:02 | POTEH | Prostate | 5869 |
| MGKWCRHCFA | HLA-B08:01 | POTEH | Prostate | 5870 |
| YAVSSRHINVI | HLA-B08:01 | POTEH | Prostate | 5871 |
| AVSSRHNVI | HLA-B08:01 | POTEH | Prostate | 5872 |
| FAWCRGSGKSNV | HLA-B08:01 | POTEH | Prostate | 5873 |
| KMGKWCRHCFA | HLA-B08:01 | POTEH | Prostate | 5874 |
| SKMGKWCRHCFA | HLA-B08:01 | POTEH | Prostate | 5875 |
| FAWCRGSG | HLA-B08:01 | POTEH | Prostate | 5876 |
| FPCCRGSGKNKV | HLA-B08:01 | POTEH | Prostate | 5877 |
| SSRHNVICQL | HLA-B08:01 | POTEH | Prostate | 5878 |
| DYKEKQIL | HLA-B08:01 | POTEH | Prostate | 5879 |
| DYKEKQILKV | HLA-B08:01 | POTEH | Prostate | 5880 |
| SDYKEKQIL | HLA-B08:01 | POTEH | Prostate | 5881 |
| SGKNKVGP | HLA-B08:01 | POTEH | Prostate | 5882 |
| DYKEKQILK | HLA-B08:01 | POTEH | Prostate | 5883 |
| KEKQILKV | HLA-B08:01 | POTEH | Prostate | 5884 |
| QILKVSSE | HLA-B08:01 | POTEH | Prostate | 5885 |
| LLSDYKEKQIL | HLA-B08:01 | POTEH | Prostate | 5886 |
| REYAVSSRHNVI | HLA-B13:02 | POTEH | Prostate | 5887 |
| REYAVSSRHNV | HLA-B13:02 | POTEH | Prostate | 5888 |
| CQLLSDYKEKQI | HLA-B13:02 | POTEH | Prostate | 5889 |
| CKWCCHCFPW | HLA-B13:02 | POTEH | Prostate | 5890 |
| AREYAVSSRHNV | HLA-B13:02 | POTEH | Prostate | 5891 |
| SDYKEKQILKV | HLA-B13:02 | POTEH | Prostate | 5892 |
| YAVSSRHNV | HLA-B13:02 | POTEH | Prostate | 5893 |
| GKWCRHCFAW | HLA-B13:02 | POTEH | Prostate | 5894 |
| YAVSSRHNVI | HLA-B13:02 | POTEH | Prostate | 5895 |
| KEKQILKV | HLA-B13:02 | POTEH | Prostate | 5896 |
| YKEKQILKV | HLA-B13:02 | POTEH | Prostate | 5897 |
| AVSSRHNVI | HLA-B13:02 | POTEH | Prostate | 5898 |
| RGSGKNKV | HLA-B13:02 | POTEH | Prostate | 5899 |
| SDYKEKQIL | HLA-B13:02 | POTEH | Prostate | 5900 |
| RHNVICQLL | HLA-B13:02 | POTEH | Prostate | 5901 |
| ALTKAVQCQ | HLA-B13:02 | POTEH | Prostate | 5902 |
| KQILKVSSE | HLA-B13:02 | POTEH | Prostate | 5903 |
| AVSSRHNV | HLA-B13:02 | POTEH | Prostate | 5904 |
| YAVSSRHNV | HLA-B46:01 | POTEH | Prostate | 5905 |
| YAVSSRHNVI | HLA-B46:01 | POTEH | Prostate | 5906 |
| FAWCRGSGK | HLA-B46:01 | POTEH | Prostate | 5907 |
| SSRHNVICQL | HLA-B46:01 | POTEH | Prostate | 5908 |
| AVSSRHNVI | HLA-B46:01 | POTEH | Prostate | 5909 |
| SGKNKVGPW | HLA-B46:01 | POTEH | Prostate | 5910 |
| FAWCRGSGKSNV | HLA-B46:01 | POTEH | Prostate | 5911 |
| SRHNVICQL | HLA-B46:01 | POTEH | Prostate | 5912 |
| FAWCRGSGKS | HLA-B46:01 | POTEH | Prostate | 5913 |
| ILKVSSEN | HLA-B46:01 | POTEH | Prostate | 5914 |
| KNKVGPWGDY | HLA-B46:01 | POTEH | Prostate | 5915 |
| NLTNGATAD | HLA-B46:01 | POTEH | Prostate | 5916 |
| YAVSSRHN | HLA-B46:01 | POTEH | Prostate | 5917 |
| KQILKVSSE | HLA-B46:01 | POTEH | Prostate | 5918 |
| ILKVSSENS | HLA-B46:01 | POTEH | Prostate | 5919 |
| YAVSSRHNV | HLA-C01:02 | POTEH | Prostate | 5920 |
| AVSSRHNVI | HLA-C01:02 | POTEH | Prostate | 5921 |
| YAVSSRHNVI | HLA-C01:02 | POTEH | Prostate | 5922 |
| RHNVICQLL | HLA-C01:02 | POTEH | Prostate | 5923 |
| LSDYKEKQI | HLA-C01:02 | POTEH | Prostate | 5924 |
| SSRHNVICQL | HLA-C01:02 | POTEH | Prostate | 5929 |
| SRHNVICQL | HLA-C01:02 | POTEH | Prostate | 5926 |
| FAWCRGSGK | HLA-C01:02 | POTEH | Prostate | 5927 |
| LSDYKEKQIL | HLA-C01:02 | POTEH | Prostate | 5928 |
| NLTNGATAD | HLA-C01:02 | POTEH | Prostate | 5929 |
| VSSRHNVI | HLA-C01:02 | POTEH | Prostate | 5930 |
| RHNVICQL | HLA-C01:02 | POTEH | Prostate | 5931 |
| ILKVSSEN | HLA-C01:02 | POTEH | Prostate | 5932 |
| YAVSSRHNV | HLA-C03:04 | POTEH | Prostate | 5933 |
| YAVSSRhNVI | HLA-C03:04 | POTEH | Prostate | 5934 |
| AVSSRHNVI | HLA-C03:04 | POTEH | Prostate | 5935 |
| FAWCRGSGKSNY | HLA-C03:04 | POTEH | Prostate | 5936 |
| FAWCRGSGK | HLA-C03:04 | POTEH | Prostate | 5937 |
| EYAVSSRHNVI | HLA-C03:04 | POTEH | Prostate | 5938 |
| EYAVSSRHNV | HLA-C03:04 | POTEH | Prostate | 5939 |
| RHNVICQLL | HLA-C03:04 | POTEH | Prostate | 5940 |
| REYAVSSRHNVI | HLA-C03:04 | POTEH | Prostate | 5941 |
| YAVSSRHN | HLA-C03:04 | POTEH | Prostate | 5942 |
| SRHNVICQL | HLA-C03:04 | POTEH | Prostate | 5943 |
| LSDYKEKQIL | HLA-C03:04 | POTEH | Prostate | 5944 |
| FAWCRGSG | HLA-C03:04 | POTEH | Prostate | 5945 |
| MGFPENLTN | HLA-C03:04 | POTEH | Prostate | 5946 |
| SDYKEKQIL | HLA-C03:04 | POTEH | Prostate | 5947 |
| RHNVICQLL | HLA-C04:01 | POTEH | Prostate | 5948 |
| FAWCRGSGK | HLA-C04:01 | POTEH | Prostate | 5949 |
| SRHNVICQL | HLA-C04:01 | POTEH | Prostate | 5950 |
| SRHNVICQLL | HLA-C04:01 | POTEH | Prostate | 5951 |
| CFAWCRGSGK | HLA-C04:01 | POTEH | Prostate | 5952 |
| YAVSSRHNV | HLA-C04:01 | POTEH | Prostate | 5953 |
| AVSSRHNVI | HLA-C04:01 | POTEH | Prostate | 5954 |
| YAVSSRHNVI | HLA-C04:01 | POTEH | Prostate | 5955 |
| MGKWCRHCFA | HLA-C04:01 | POTEH | Prostate | 5956 |
| DYKEKQIL | HLA-C04:01 | POTEH | Prostate | 5957 |
| RHNVICQL | HLA-C04:01 | POTEH | Prostate | 5958 |
| LSDYKEKQI | HLA-C04:01 | POTEH | Prostate | 5959 |
| LSDYKEKQIL | HLA-C04:01 | POTEH | Prostate | 5960 |
| YKEKQILKV | HLA-C04:01 | POTEH | Prostate | 5961 |
| LTNGATADN | HLA-C04:01 | POTEH | Prostate | 5962 |
| LTNGATAD | HLA-C04:01 | POTEH | Prostate | 5963 |
| SRHNVICQL | HLA-C07:01 | POTEH | Prostate | 5964 |
| ISRHNVICQLL | HLA-C07:01 | POTEH | Prostate | 5965 |
| SSRHNVICQL | HLA-C07:01 | POTEH | Prostate | 5966 |
| RHINVICQLL | HLA-C07:01 | POTEH | Prostate | 5967 |
| YAVSSRHNV | HLA-C07:01 | POTEH | Prostate | 5968 |
| YKEKQILKV | HLA-C07:01 | POTEH | Prostate | 5969 |
| SSRHNVICQLL | HLA-C07:01 | POTEH | Prostate | 5970 |
| CRQSGKNKV | HLA-C07:01 | POTEH | Prostate | 5971 |
| AVSSRHNVI | HLA-C07:01 | POTEH | Prostate | 5972 |
| SDYKEKQIL | HLA-C07:01 | POTEH | Prostate | 5973 |
| LTNGATAD | HLA-C07:01 | POTEH | Prostate | 5974 |
| DYKEKQIL | HLA-C07:01 | POTEH | Prostate | 5975 |
| GSGKNKVGP | HLA-C07:01 | POTEH | Prostate | 5976 |
| LSDYKEKQIL | HLA-C07:01 | POTEH | Prostate | 5977 |
| KNKVGPWGD | HLA-C07:01 | POTEH | Prostate | 5978 |
| RHNVICQL | HLA-C07:01 | POTEH | Prostate | 5979 |
| HCFAWCRGS | HLA-C07:01 | POTEH | Prostate | 5980 |
| SRHNVICQL | HLA-C07:02 | POTEH | Prostate | 5981 |
| SRHNVICQLL | HLA-C07:02 | POTEH | Prostate | 5982 |
| RHNVICQLL | HLA-C07:02 | POTEH | Prostate | 5983 |
| SSRHNVICQL | HLA-C07:02 | POTEH | Prostate | 5984 |
| YAVSSRHNV | HLA-C07:02 | POTEH | Prostate | 5985 |
| SSRHNVICQLL | HLA-C07:02 | POTEH | Prostate | 5986 |
| AVSSRHNVI | HLA-C07:02 | POTEH | Prostate | 5987 |
| YKEKQILKV | HLA-C07:02 | POTEH | Prostate | 5988 |
| CRHCFAWCR | HLA-C07:02 | POTEH | Prostate | 5989 |
| DYKEKQIL | HLA-C07:02 | POTEH | Prostate | 5990 |
| NKVGPWGDY | HLA-C07:02 | POTEH | Prostate | 5991 |
| SRHNVICQ | HLA-C07:02 | POTEH | Prostate | 5992 |
| AREYAVSSR | HLA-C07:02 | POTEH | Prostate | 5993 |
| SDYKEKQIL | HLA-C07:02 | POTEH | Prostate | 5994 |
| RHNVICQL | HLA-C07:02 | POTEH | Prostate | 5995 |
| STGEKGFRY | HLA-A01:01 | PPIAL4G | Colorectal; Uterine | 5996 |
| IVEAMEHFGY | HLA-A01:01 | PPIALAG | Colorectal; Uterine | 5997 |
| LSTGEKGFRY | HLA-A01:01 | PPIAL4G | Colorectal; Uterine | 5998 |
| SVIFFDITV | HLA-A02:01 | PPIAL4G | Colorectal; Uterine | 5999 |
| MVNSVIFFDI | HLA-A02:01 | PPIAL4G | Colorectal; Uterine | 6000 |
| NSVIFFDITV | HLA-A02:01 | PPIAL4G | Colorectal; Uterine | 6001 |
| MVNSVIFFDITV | HLA-A02:01 | PPIAL4G | Colorectal; Uterine | 6002 |
| SVIFFDITVD | HLA-A02:01 | PPIAL4G | Colorectal; Uterine | 6003 |
| WLDGKHVAF | HLA-A02:01 | PPIAL4G | Colorectal; Uterine | 6004 |
| WLDGKHVAPGKV | HLA-A02:01 | PPIAL4G | Colorectal; Uterine | 6005 |
| VIFFDITV | HLA-A02:01 | PPIAL4G | Colorectal; Uterine | 6006 |
| VNSVIFFDITV | HLA-A02:01 | PPIAL4G | Colorectal; Uterine | 6007 |
| KVKERVNIV | HLA-A02:01 | PPIAL4G | Colorectal; Uterine | 6008 |
| WLDGKHVA | HLA-A02:01 | PPIAL4G | Colorectal; Uterine | 6009 |
| FADKIPKTA | HLA-A02:01 | PPIAL4G | Colorectal; Uterine | 6010 |
| FDDENLIRK | HLA-A02:01 | PPIAL4G | Colorectal; Uterine | 6011 |
| WLDGKHVAFG | HLA-A02:01 | PPIAL4G | Colorectal; Uterine | 6012 |
| EWLDGKHVA | HLA-A02:01 | PPIAL4G | Colorectal; Uterine | 6013 |
| RISIKQFADK | HLA-A03:01 | PPIAL4G | Colorectal; Uterine | 6014 |
| AMEHFGYRNSK | HLA-A03:01 | PPIAL4G | Colorectal; Uterine | 6015 |
| ALSTGEKGFRYK | HLA-A03:01 | PPIAL4G | Colorectal; Uterine | 6016 |
| KQFADKIPK | HLA-A03:01 | PPIAL4G | Colorectal; Uterine | 6017 |
| STGEKGFRYK | HLA-A03:01 | PPIAL4G | Colorectal; Uterine | 6018 |
| STGEKGFRYK | HLA-A11:01 | PPIAL4G | Colorectal; Uterine | 6019 |
| SVIFFDITVDGK | HLA-A11:01 | PPIAL4G | Colorectal; Uterine | 6020 |
| ISIKQFADK | HLA-A11:01 | PPIAL4G | Colorectal; Uterine | 6021 |
| ITVDGKPLGR | HLA-A11:01 | PPIAL4G | Colorectal; Uterine | 6022 |
| TVDGKPLGR | HLA-A11:01 | PPIAL4G | Colorectal; Uterine | 6023 |
| RYKGSCFHRI | HLA-A24:02 | PPIAL4G | Colorectal; Uterine | 6024 |
| RYKGSCFHRII | HLA-A24:02 | PPIAL4G | Colorectal; Uterine | 6025 |
| FRYKGSCFHRI | HLA-A24:02 | PPIAL4G | Colorectal; Uterine | 6026 |
| EWLDGKHVAF | HLA-A24:02 | PPIAL4G | Colorectal; Uterine | 6027 |
| KIPKTAENF | HLA-A24:02 | PPIAL4G | Colorectal; Uterine | 6028 |
| KVKERVNIV | HLA-A30:01 | PPIAL4G | Colorectal; Uterine | 6029 |
| GYRNSKTSK | HLA-A30:01 | PPIAL4G | Colorectal; Uterine | 6030 |
| KQFADKIPK | HLA-A30:01 | PPIAL4G | Colorectal; Uterine | 6031 |
| STGEKGFRYK | HLA-A30:01 | PPIAL4G | Colorectal; Uterine | 6032 |
| EAMEHFGYR | HLA-A33:03 | PPIAL4G | Colorectal; Uterine | 6033 |
| VEAMEHFGYR | HLA-A33:03 | PPIAL4G | Colorectal; Uterine | 6034 |
| FRYKGSCFHR | HLA-A33:03 | PPIAL4G | Colorectal; Uterine | 6035 |
| VAFGKVKER | HLA-A33:03 | PPIAL4G | Colorectal; Uterine | 6036 |
| TVDGKPLGR | HLA-A33:03 | PPIAL4G | Colorectal; Uterine | 6037 |
| IPKTAENFRAL | HLA-B07:02 | PPIAL4G | Colorectal; Uterine | 6038 |
| KPLGRISI | HLA-B07:02 | PPIAL4G | Colorectal; Uterine | 6039 |
| HPNGTGDKSI | HLA-B07:02 | PPIALAG | Colorectal; Uterine | 6040 |
| KPLGRISIK | HLA-B07:02 | PPIAL4G | Colorectal; Uterine | 6041 |
| FGKVKERVNI | HLA-B08:01 | PPIAL4G | Colorectal; Uterine | 6042 |
| LIRKHTGSGI | HLA-B08:01 | PPIAL4G | Colorectal; Uterine | 6043 |
| KVKERVNIV | HLA-B08:01 | PPIAL4G | Colorectal; Uterine | 6044 |
| ENLIRKHTG | HLA-B08:01 | PPIAL4G | Colorectal; Uterine | 6045 |
| WLDGKHVAF | HLA-B08:01 | PPIAL4G | Colorectal; Uterine | 6046 |
| MVNSVIFFDI | HLA-B13:02 | PPIAL4G | Colorectal; Uterine | 6047 |
| VNSVIFFDI | HLA-B13:02 | PPIAL4G | Colorectal; Uterine | 6048 |
| SVIFFDITV | HLA-B13:02 | PPIAL4G | Colorectal; Uterine | 6049 |
| RNSKTSKKI | HLA-B13:02 | PPIAL4G | Colorectal; Uterine | 6050 |
| AFGKVKERV | HLA-B13:02 | PPIAL4G | Colorectal; Uterine | 6051 |
| ITIADCGQF | HLA-B46:01 | PPIAL4G | Colorectal; Uterine | 6052 |
| HTGSGILSM | HLA-B46:01 | PPIAL4G | Colorectal; Uterine | 6053 |
| NIVEAMEHF | HLA-B46:01 | PPIAL4G | Colorectal; Uterine | 6054 |
| VIFFDITVD | HLA-B46:01 | PPIAL4G | Colorectal; Uterine | 6055 |
| STGEKGFRY | HLA-B46:01 | PPIAL4G | Colorectal; Uterine | 6056 |
| WLDGKHVAF | HLA-B46:01 | PPIAL4G | Colorectal; Uterine | 6057 |
| WLDGKHVAF | HLA-C01:02 | PPIAL4G | Colorectal; Uterine | 6058 |
| HTGSGILSM | HLA-C01:02 | PPIAL4G | Colorectal; Uterine | 6059 |
| KIPKTAENF | HLA-C01:02 | PPIAL4G | Colorectal; Uterine | 6060 |
| KIPKTAENFRAL | HLA-C01:02 | PPIAL4G | Colorectal; Uterine | 6061 |
| KIPKTAEN | HLA-C01:02 | PPIAL40 | Colorectal; Uterine | 6062 |
| HTGSGILSM | HLA-C03:04 | PPIAL4G | Colorectal; Uterine | 6063 |
| ITIADCGQF | HLA-C03:04 | PPIAL4G | Colorectal; Uterine | 6064 |
| WLDGKHVAF | HLA-C03:04 | PPIAL4G | Colorectal; Uterine | 6065 |
| FADKIPKTA | HLA-C03:04 | PPIAL4G | Colorectal; Uterine | 6066 |
| ITVDGKPL | HLA-C03:04 | PPIAL40 | Colorectal; Uterine | 6067 |
| FDITVDGKPL | HLA-C03:04 | PPIAL4G | Colorectal; Uterine | 6068 |
| WLDGKHVAF | HLA-C04:01 | PPIAL4G | Colorectal; Uterine | 6069 |
| FFDITVDGKPL | HLA-C04:01 | PPIAL4G | Colorectal; Uterine | 6070 |
| FFDITVDGK | HLA-C04:01 | PPIAL4G | Colorectal; Uterine | 6071 |
| KFDDENLI | HLA-C04:01 | PPIAL4G | Colorectal; Uterine | 6072 |
| FFDITVDG | HLA-C04:01 | PPIAL4G | Colorectal; Uterine | 6073 |
| FRYKGSCFH | HLA-C07:01 | PPIAL4G | Colorectal; Uterine | 6074 |
| FRYKGSCFHRII | HLA-C07:01 | PPIAL4G | Colorectal; Uterine | 6075 |
| FRYKGSCFHRI | HLA-C07:01 | PPIAL4G | Colorectal; Uterine | 6076 |
| HTGSGILSM | HLA-C07:01 | PPIAL4G | Colorectal; Uterine | 6077 |
| SIYGEKFDD | HLA-C07:01 | PPIAL4G | Colorectal; Uterine | 6078 |
| IFFDITVD | HLA-C07:01 | PPIAL4G | Colorectal; Uterine | 6079 |
| FRYKGSCFH | HLA-C07:02 | PPIAL4G | Colorectal; Uterine | 6080 |
| FRYKGSCF | HLA-C07:02 | PPIAL4G | Colorectal; Uterine | 6081 |
| FRYKGSCFHRII | HLA-C07:02 | PPIAL4G | Colorectal; Uterine | 6082 |
| ERVNIVEAM | HLA-C07:02 | PPIAL4G | Colorectal; Uterine | 6083 |
| YRNSKTSK | HLA-C07:02 | PPIAL4G | Colorectal; Uterine | 6084 |
| LSNLTHVLY | HLA-A01:01 | PRAME | Lung | 6085 |
| | | | squam.; Melanoma; | |
| | | | Ovarian; Uterine | |
| CSQLTTLSFY | HLA-A01:01 | PRAME | Lung | 6086 |
| | | | squam.; Melanoma; | |
| | | | Ovarian; Uterine | |
| WSGNRASLY | HLA-A01:01 | PRAME | Lung | 6087 |
| | | | squam.; Melanoma; | |
| | | | Ovarian; Uterine | |
| ITDDQLLAL | HLA-A01:01 | PRAME | Lung | 6088 |
| | | | squam.; Melanoma; | |
| | | | Ovarian; Uterine | |
| YVDSLFFLR | HLA-A01:01 | PRAME | Lung | 6089 |
| | | | squam.; Melanoma; | |
| | | | Ovarian; Uterine | |
| NLTHVLYPV | HLA-A02:01 | PRAME | Lung | 6090 |
| | | | squam.; Melanoma; | |
| | | | Ovarian; Uterine | |
| ALYVDSLFFL | HLA-A02:01 | PRAME | Lung | 6091 |
| | | | squam.; Melanoma; | |
| | | | Ovarian; Uterine | |
| TLAKFSPYL | HLA-A02:01 | PRAME | Lung | 6092 |
| | | | squam.; Melanoma; | |
| | | | Ovarian; Uterine | |
| QLLALLPSL | HLA-A02:01 | PRAME | Lung | 6093 |
| | | | squam.; Melanoma; | |
| | | | Ovarian; Uterine | |
| WLLHHSWVWV | HLA-A02:01 | PRAME | Lung | 6094 |
| | | | squam.; Melanoma; | |
| | | | Ovarian; Uterine | |
| SLLQHLIGL | HLA-A02:01 | PRAME | Lung | 6095 |
| | | | squam.; Melanoma; | |
| | | | Ovarian; Uterine | |
| SNLTHVLYPV | HLA-A02:01 | PRAME | Lung | 6096 |
| | | | squam.; Melanoma; | |
| | | | Ovarian; Uterine | |
| YELQEALISL | HLA-A02:01 | PRAME | Lung | 6097 |
| | | | squam.; Melanoma; | |
| | | | Ovarian; Uterine | |
| VLDGLDVLL | HLA-A02:01 | PRAME | Lung | 6098 |
| | | | squam.; Melanoma; | |
| | | | Ovarian; Uterine | |
| ALISLPHGL | HLA-A02:01 | PRAME | Lung | 6099 |
| | | | squam.; Melanoma; | |
| | | | Ovarian; Uterine | |
| ALQSLLQHL | HLA-A02:01 | PRAME | Lung | 6100 |
| | | | squam.; Melanoma; | |
| | | | Ovarian; Uterine | |
| RLSEGDVMHL | HLA-A02:01 | PRAME | Lung | 6101 |
| | | | squam.; Melanoma; | |
| | | | Ovarian; Uterine | |
| ALLERASATL | HLA-A02:01 | PRAME | Lung | 6102 |
| | | | squam.; Melanoma; | |
| | | | Ovarian; Uterine | |
| ELQEALISL | HLA-A02:01 | PRAME | Lung | 6103 |
| | | | squam.; Melanoma; | |
| | | | Ovarian; Uterine | |
| RLDQLLRHV | HLA-A02:01 | PRAME | Lung | 6104 |
| | | | squam.; Melanoma; | |
| | | | Ovarian; Uterine | |
| VLYPVPLESY | HLA-A03:01 | PRAME | Lung | 6105 |
| | | | squam.; Melanoma; | |
| | | | Ovarian; Uterine | |
| KIFAMPMQDIK | HLA-A03:01 | PRAME | Lung | 6106 |
| | | | squam.; Melanoma; | |
| | | | Ovarian; Uterine | |
| VELAGQSLLK | HLA-A03:01 | PRAME | Lung | 6107 |
| | | | squam.; Melanoma; | |
| | | | Ovarian; Uterine | |
| ELFSYLIEK | HLA-A03:01 | PRAME | Lung | 6108 |
| | | | squam.; Melanoma; | |
| | | | Ovarian; Uterine | |
| VLYPVPLES | HLA-A03:01 | PRAME | Lung | 6109 |
| | | | squam.; Melanoma; | |
| | | | Ovarian; Uterine | |
| ASSYISPEK | HLA-A11:01 | PRAME | Lung | 6110 |
| | | | squam.; Melanoma; | |
| | | | Ovarian; Uterine | |
| CTWKLPTLAK | HLA-A11:01 | PRAME | Lung | 6111 |
| | | | squam.; Melanoma; | |
| | | | Ovarian; Uterine | |
| ISMSVWTSPR | HLA-A11:01 | PRAME | Lung | 6112 |
| | | | squam.; Melanoma; | |
| | | | Ovarian; Uterine | |
| YVDSLFFLR | HLA-A11:01 | PRAME | Lung | 6113 |
| | | | squam.; Melanoma; | |
| | | | Ovarian; Uterine | |
| EVLVDLFLK | HLA-A11:01 | PRAME | Lung | 6114 |
| | | | squam.; Melanoma; | |
| | | | Ovarian; Uterine | |
| ELFSYLIEK | HLA-A11:01 | PRAME | Lung | 6115 |
| | | | squam.; Melanoma; | |
| | | | Ovarian; Uterine | |
| QYIAQFTSQF | HLA-A24:02 | PRAME | Lung | 6116 |
| | | | squam.; Melanoma; | |
| | | | Ovarian; Uterine | |
| RWLLHHSWVW | HLA-A24:02 | PRAME | Lung | 6117 |
| | | | squam.; Melanoma; | |
| | | | Ovarian; Uterine | |
| TWKLPTLAKF | HLA-A24:02 | PRAME | Lung | 6118 |
| | | | squam.; Melanoma; | |
| | | | Ovarian; Uterine | |
| LYVDSLFFL | HLA-A24:02 | PRAME | Lung | 6119 |
| | | | squam.; Melanoma; | |
| | | | Ovarian; Uterine | |
| PYLGQMINL | HLA-A24:02 | PRAME | Lung | 6120 |
| | | | squam.; Melanoma; | |
| | | | Ovarian; Uterine | |
| SYEDIHGTL | HLA-A24:02 | PRAME | Lung | 6121 |
| | | | squam.; Melanoma; | |
| | | | Ovarian; Uterine | |
| QSRYISMSV | HLA-A30:01 | PRAME | Lung | 6122 |
| | | | squam.; Melanoma; | |
| | | | Ovarian; Uterine | |
| KLKIPAMPM | HLA-A30:01 | PRAME | Lung | 6123 |
| | | | squam.; Melanoma; | |
| | | | Ovarian; Uterine | |
| IQSRYISMSV | HLA-A30:01 | PRAME | Lung | 6124 |
| | | | squam.; Melanoma; | |
| | | | Ovarian; Uterine | |
| ASSYISPEK | HLA-A30:01 | PRAME | Lung | 6125 |
| | | | squam.; Melanoma; | |
| | | | Ovarian; Uterine | |
| TWKLPTLAK | HLA-A30:01 | PRAME | Lung | 6126 |
| | | | squam.; Melanoma; | |
| | | | Ovarian; Uterine | |
| RLRELLCEL | HLA-A30:01 | PRAME | Lung | 6127 |
| | | | squam.; Melanoma; | |
| | | | Ovarian; Uterine | |
| HSWVWVPFR | HLA-A33:03 | PRAME | Lung | 6128 |
| | | | squam.; Melanoma; | |
| | | | Ovarian; Uterine | |
| SMSVWTSPR | HLA-A33:03 | PRAME | Lung | 6129 |
| | | | squam.; Melanoma; | |
| | | | Ovarian; Uterine | |
| ISMSVWTSPR | HLA-A33:03 | PRAME | Lung | 6130 |
| | | | squam.; Melanoma; | |
| | | | Ovarian; Uterine | |
| ETLSITNCR | HLA-A33:03 | PRAME | Lung | 6131 |
| | | | squam.; Melanoma; | |
| | | | Ovarian; Uterine | |
| DSLFFLRGR | HLA-A33:03 | PRAME | Lung | 6132 |
| | | | squam.; Melanoma; | |
| | | | Ovarian; Uterine | |
| YVDSLFFLR | HLA-A33:03 | PRAME | Lung | 6133 |
| | | | squam.; Melanoma; | |
| | | | Ovarian; Uterine | |
| RPRRWKLQVL | HLA-B07:02 | PRAME | Lung | 6134 |
| | | | squam.; Melanoma; | |
| | | | Ovarian; Uterine | |
| LPRGLGRWL | HLA-B07:02 | PRAME | Lung | 6135 |
| | | | squam.; Melanoma; | |
| | | | Ovarian; Uterine | |
| RPRRWKLQV | HLA-B07:02 | PRAME | Lung | 6136 |
| | | | squam.; Melanoma; | |
| | | | Ovarian; Uterine | |
| LPRELFPPL | HLA-B07:02 | PRAME | Lung | 6137 |
| | | | squam.; Melanoma; | |
| | | | Ovarian; Uterine | |
| SPYLGQMINL | HLA-B07:02 | PRAME | Lung | 6138 |
| | | | squam.; Melanoma; | |
| | | | Ovarian; Uterine | |
| SPSVSQLSVL | HLA-B07:02 | PRAME | Lung | 6139 |
| | | | squam.; Melanoma; | |
| | | | Ovarian; Uterine | |
| YLHARLREL | HLA-B08:01 | PRAME | Lung | 6140 |
| | | | squam.; Melanoma; | |
| | | | Ovarian; Uterine | |
| MINLRRLLL | HLA-B08:01 | PRAME | Lung | 6141 |
| | | | squam.; Melanoma; | |
| | | | Ovarian; Uterine | |
| FLRGRLDQL | HLA-B08:01 | PRAME | Lung | 6142 |
| | | | squam.; Melanoma; | |
| | | | Ovarian; Uterine | |
| KVKRKKNVL | HLA-B08:01 | PRAME | Lung | 6143 |
| | | | squam.; Melanoma; | |
| | | | Ovarian; Uterine | |
| REYELQEALI | HLA-B13:02 | PRAME | Lung | 6144 |
| | | | squam.; Melanoma; | |
| | | | Ovarian; Uterine | |
| CDELFSYLI | HLA-B13:02 | PRAME | Lung | 6145 |
| | | | squam.; Melanoma; | |
| | | | Ovarian; Uterine | |
| LQSLLQHLI | HLA-B13:02 | PRAME | Lung | 6146 |
| | | | squam.; Melanoma; | |
| | | | Ovarian; Uterine | |
| AEQPFIPVEV | HLA-B13:02 | PRAME | Lung | 6147 |
| | | | squam.; Melanoma; | |
| | | | Ovarian; Uterine | |
| EQPFIPVEV | HLA-B13:02 | PRAME | Lung | 6148 |
| | | | squam.; Melanoma; | |
| | | | Ovarian; Uterine | |
| RLCCKKLKI | HLA-B13:02 | PRAME | Lung | 6149 |
| | | | squam.; Melanoma; | |
| | | | Ovarian; Uterine | |
| RLWGSIQSR | HLA-B13:02 | PRAME | Lung | 6150 |
| | | | squam.; Melanoma; | |
| | | | Ovarian; Uterine | |
| YIAQFTSQF | HLA-B46:01 | PRAME | Lung | 6151 |
| | | | squam.; Melanoma; | |
| | | | Ovarian; Uterine | |
| LSHIHASSY | HLA-B46:01 | PRAME | Lung | 6152 |
| | | | squam.; Melanoma; | |
| | | | Ovarian; Uterine | |
| FAMPMQDIKM | HLA-B46:01 | PRAME | Lung | 6153 |
| | | | squam.; Melanoma; | |
| | | | Ovarian; Uterine | |
| VLYPVPLESY | HLA-B46:01 | PRAME | Lung | 6154 |
| | | | squam.; Melanoma; | |
| | | | Ovarian; Uterine | |
| AAFDGRHSQ | HLA-B46:01 | PRAME | Lung | 6155 |
| | | | squam.; Melanoma; | |
| | | | Ovarian; Uterine | |
| VSPEPLQAL | HLA-C01:02 | PRAME | Lung | 6156 |
| | | | squam.; Melanoma; | |
| | | | Ovarian; Uterine | |
| YLHARLREL | HLA-C01:02 | PRAME | Lung | 6157 |
| | | | squam.; Melanoma; | |
| | | | Ovarian; Uterine | |
| ITDDQLLAL | HLA-C01:02 | PRAME | Lung | 6158 |
| | | | squam.; Melanoma; | |
| | | | Ovarian; Uterine | |
| TSPRRLVEL | HLA-C01:02 | PRAME | Lung | 6159 |
| | | | squam.; Melanoma; | |
| | | | Ovarian; Uterine | |
| AWPFTCLPL | HLA-C01:02 | PRAME | Lung | 6160 |
| | | | squam.; Melanoma; | |
| | | | Ovarian; Uterine | |
| ISISALQSL | HLA-C03:04 | PRAME | Lung | 6161 |
| | | | squam.; Melanoma; | |
| | | | Ovarian; Uterine | |
| LAIAALELL | HLA-C03:04 | PRAME | Lung | 6162 |
| | | | squam.; Melanoma; | |
| | | | Ovarian; Uterine | |
| HVMNPLETL | HLA-C03:04 | PRAME | Lung | 6163 |
| | | | squam.; Melanoma; | |
| | | | Ovarian; Uterine | |
| LAIAALEL | HLA-C03:04 | PRAME | Lung | 6164 |
| | | | squam.; Melanoma; | |
| | | | Ovarian; Uterine | |
| FTSQFLSL | HLA-C03:04 | PRAME | Lung | 6165 |
| | | | squam.; Melanoma; | |
| | | | Ovarian; Uterine | |
| FYDPEPILCPCF | HLA-C04:01 | PRAME | Lung | 6166 |
| | | | squam.; Melanoma; | |
| | | | Ovarian; Uterine | |
| SYEDIHGTL | HLA-C04:01 | PRAME | Lung | 6167 |
| | | | squam.; Melanoma; | |
| | | | Ovarian; Uterine | |
| HHSWVWVPF | HLA-C04:01 | PRAME | Lung | 6168 |
| | | | squam.; Melanoma; | |
| | | | Ovarian; Uterine | |
| FYDPEPILC | HLA-C04:01 | PRAME | Lung | 6169 |
| | | | squam.; Melanoma; | |
| | | | Ovarian; Uterine | |
| FYDPEPIL | HLA-C04:01 | PRAME | Lung | 6170 |
| | | | squam.; Melanoma; | |
| | | | Ovarian; Uterine | |
| VFDECGITD | HLA-C04:01 | PRAME | Lung | 6171 |
| | | | squam.; Melanoma; | |
| | | | Ovarian; Uterine | |
| YLHARLREL | HLA-C07:01 | PRAME | Lung | 6172 |
| | | | squam.; Melanoma; | |
| | | | Ovarian; Uterine | |
| KRKKNVLRL | HLA-C07:01 | PRAME | Lung | 6173 |
| | | | squam.; Melanoma; | |
| | | | Ovarian; Uterine | |
| SFYGNSISI | HLA-C07:01 | PRAME | Lung | 6174 |
| | | | squam.; Melanoma; | |
| | | | Ovarian; Uterine | |
| LHLERLAYL | HLA-C07:01 | PRAME | Lung | 6175 |
| | | | squam.; Melanoma; | |
| | | | Ovarian; Uterine | |
| RRWKLQVL | HLA-C07:01 | PRAME | Lung | 6176 |
| | | | squam.; Melanoma; | |
| | | | Ovarian; Uterine | |
| RRWKLQVLDL | HLA-C07:01 | PRAME | Lung | 6177 |
| | | | squam.; Melanoma; | |
| | | | Ovarian; Uterine | |
| YLHARLREL | HLA-C07:02 | PRAME | Lung | 6178 |
| | | | squam.; Melanoma; | |
| | | | Ovarian; Uterine | |
| LYVDSLFFL | HLA-C07:02 | PRAME | Lung | 6179 |
| | | | squam.; Melanoma; | |
| | | | Ovarian; Uterine | |
| LHLERLAYL | HLA-C07:02 | PRAME | Lung | 6180 |
| | | | squam.; Melanoma; | |
| | | | Ovarian; Uterine | |
| KRKKNVLRL | HLA-C07:02 | PRAME | Lung | 6181 |
| | | | squam.; Melanoma; | |
| | | | Ovarian; Uterine | |
| FYDPEPIL | HLA-C07:02 | PRAME | Lung | 6182 |
| | | | squam.; Melanoma; | |
| | | | Ovarian; Uterine | |
| SYEDIHGTL | HLA-C07:02 | PRAME | Lung | 6183 |
| | | | squam.; Melanoma; | |
| | | | Ovarian; Uterine | |
| KSSANWMRY | HLA-A01:01 | PRDM7 | Melanoma | 6184 |
| LSEPQDDDY | HLA-A01:01 | PRDM7 | Melanoma | 6185 |
| ISEPQDDDYLY | HLA-A01:01 | PRDM7 | Melanoma | 6186 |
| ITEDEEAANSGY | HLA-A01:01 | PRDM7 | Melanoma | 6187 |
| ASDLPLGLH | HLA-A01:01 | PRDM7 | Melanoma | 6188 |
| YLYCEMCQNFFI | HLA-A02:01 | PRDM7 | Melanoma | 6189 |
| NMWNAITPL | HLA-A02:01 | PRDM7 | Melanoma | 6190 |
| KMNYNALITY | HLA-A02:01 | PRDM7 | Melanoma | 6191 |
| FSVNMWNAITPL | HLA-A02:01 | PRDM7 | Melanoma | 6192 |
| SLLSELPRTI | HLA-A02:01 | PRDM7 | Melanoma | 6193 |
| VLLMGPLHL | HLA-A02:01 | PRDM7 | Melanoma | 6194 |
| VNMWNAITPL | HLA-A02:01 | PRDM7 | Melanoma | 6195 |
| SMSLMLSGL | HLA-A02:01 | PRDM7 | Melanoma | 6196 |
| SLLSELPRT | HLA-A02:01 | PRDM7 | Melanoma | 6197 |
| LMLSGLEKSKI | HLA-A02:01 | PRDM7 | Melanoma | 6198 |
| ALITVGLRA | HLA-A02:01 | PRDM7 | Melanoma | 6199 |
| LLSELPRTI | HLA-A02:01 | PRDM7 | Melanoma | 6200 |
| LLMGPLHL | HLA-A02:01 | PRDM7 | Melanoma | 6201 |
| ELSGTPNLL | HLA-A02:01 | PRDM7 | Melanoma | 6202 |
| SLRELSGTPNL | HLA-A02:01 | PRDM7 | Melanoma | 6203 |
| SGDEYGQEL | HLA-A02:01 | PRDM7 | Melanoma | 6204 |
| MSLMLSGLFK | HLA-A03:01 | PRDM7 | Melanoma | 6205 |
| SLMLSGLFK | HLA-A03:01 | PRDM7 | Melanoma | 6206 |
| KMYSLRERK | HLA-A03:01 | PRDM7 | Melanoma | 6207 |
| ILIHAAVMTK | HLA-A03:01 | PRDM7 | Melanoma | 6208 |
| SGYSWLITK | HLA-A03:01 | PRDM7 | Melanoma | 6209 |
| AVMTKPKVK | HLA-A03:01 | PRDM7 | Melanoma | 6210 |
| SELPRTICK | HLA-A03:01 | PRDM7 | Melanoma | 6211 |
| MSLMLSGLFK | HLA-A11:01 | PRDM7 | Melanoma | 6212 |
| SLMLSGLFK | HLA-A11:01 | PRDM7 | Melanoma | 6213 |
| SSANWMRTK | HLA-A11:01 | PRDM7 | Melanoma | 6214 |
| SGYSWLITK | HLA-A11:01 | PRDM7 | Melanoma | 6215 |
| AVMTKPKVK | HLA-A11:01 | PRDM7 | Melanoma | 6216 |
| AHGPPTFVK | HLA-A11:01 | PRDM7 | Melanoma | 6217 |
| LYCEMCQNF | HLA-A24:02 | PRDM7 | Melanoma | 6218 |
| YLYCEMCQNF | HLA-A24:02 | PRDM7 | Melanoma | 6219 |
| LYCEMCQNFF | HLA-A24:02 | PRDM7 | Melanoma | 6220 |
| HVPHAVWPF | HLA-A24:02 | PRDM7 | Melanoma | 6221 |
| VWPFQVKNF | HLA-A24:02 | PRDM7 | Melanoma | 6222 |
| AFKDISIYF | HLA-A24:02 | PRDM7 | Melanoma | 6223 |
| RIRSGNILI | HLA-A30:01 | PRDM7 | Melanoma | 6224 |
| KTRYRNVKM | HLA-A30:01 | PRDM7 | Melanoma | 6225 |
| RCVRTSSLTA | HLA-A30:01 | PRDM7 | Melanoma | 6226 |
| RTKARDPSM | HLA-A30:01 | PRDM7 | Melanoma | 6227 |
| RYRNVKMNY | HLA-A30:01 | PRDM7 | Melanoma | 6228 |
| AFKDISIYF | HLA-A30:01 | PRDM7 | Melanoma | 6229 |
| RLKLELRRK | HLA-A30:01 | PRDM7 | Melanoma | 6230 |
| HAVWPFQVK | HLA-A30:01 | PRDM7 | Melanoma | 6231 |
| QVKPPWMAFR | HLA-A33:03 | PRDM7 | Melanoma | 6232 |
| WMRYVNCAR | HLA-A33:03 | PRDM7 | Melanoma | 6233 |
| FQYHRQIFYR | HLA-A33:03 | PRDM7 | Melanoma | 6234 |
| NLVAFQYHR | HLA-A33:03 | PRDM7 | Melanoma | 6235 |
| EMGDWEKTRYR | HLA-A33:03 | PRDM7 | Melanoma | 6236 |
| QYHRQIFYR | HLA-A33:03 | PRDM7 | Melanoma | 6237 |
| YSWLITKGR | HLA-A33:03 | PRDM7 | Melanoma | 6238 |
| EMGDWEKTR | HLA-A33:03 | PRDM7 | Melanoma | 6239 |
| HPNRSALSL | HLA-B07:02 | PRDM7 | Melanoma | 6240 |
| RPAFMCHRRQAI | HLA-B07:02 | PRDM7 | Melanoma | 6241 |
| KARDPSMSL | HLA-B07:02 | PRDM7 | Melanoma | 6242 |
| LPRGSESGAAL | HLA-B07:02 | PRDM7 | Melanoma | 6243 |
| GPSGIPQAGL | HLA-B07:02 | PRDM7 | Melanoma | 6244 |
| KPMVSEPL | HLA-B07:02 | PRDM7 | Melanoma | 6245 |
| IPQAGLGVW | HLA-B07:02 | PRDM7 | Melanoma | 6246 |
| FMCHRRQAI | HLA-B08:01 | PRDM7 | Melanoma | 6247 |
| NVKMNYNAL | HLA-B08:01 | PRDM7 | Melanoma | 6248 |
| IVRCVRTSSL | HLA-B08:01 | PRDM7 | Melanoma | 6249 |
| WMRTKARDPSM | HLA-B08:01 | PRDM7 | Melanoma | 6250 |
| HSRLKLEL | HLA-B08:01 | PRDM7 | Melanoma | 6251 |
| NALITVGL | HLA-B08:01 | PRDM7 | Melanoma | 6252 |
| DGKDKSSA | HLA-B08:01 | PRDM7 | Melanoma | 6253 |
| CEMCQNFFI | HLA-B13:02 | PRDM7 | Melanoma | 6254 |
| YCEMCQNFFI | HLA-B13:02 | PRDM7 | Melanoma | 6255 |
| MNYNALITV | HLA-B13:02 | PRDM7 | Melanoma | 6256 |
| YLYCEMCQNFFI | HLA-B13:02 | PRDM7 | Melanoma | 6257 |
| EQSKHQKI | HLA-B13:02 | PRDM7 | Melanoma | 6258 |
| RQIFYRTCRV | HLA-B13:02 | PRDM7 | Melanoma | 6259 |
| RSIHVPHAV | HLA-B13:02 | PRDM7 | Melanoma | 6260 |
| RQAIKLQV | HLA-B13:02 | PRDM7 | Melanoma | 6261 |
| FSVNMWNAI | HLA-B46:01 | PRDM7 | Melanoma | 6262 |
| FTKEEWAEM | HLA-B46:01 | PRDM7 | Melanoma | 6263 |
| MSLMLSGLF | HLA-B46:01 | PRDM7 | Melanoma | 6264 |
| FQVKNFSVNM | HLA-B46:01 | PRDM7 | Melanoma | 6265 |
| QVKPPWMAF | HLA-B46:01 | PRDM7 | Melanoma | 6266 |
| KARDPSMSL | HLA-B46:01 | PRDM7 | Melanoma | 6267 |
| SSIEPAESI | HLA-B46:01 | PRDM7 | Melanoma | 6268 |
| SLRERKGHAY | HLA-B46:01 | PRDM7 | Melanoma | 6269 |
| RTSSLTAVI | HLA-C01:02 | PRDM7 | Melanoma | 6270 |
| KARDPSMSL | HLA-C01:02 | PRDM7 | Melanoma | 6271 |
| FSVNMWNAI | HLA-C01:02 | PRDM7 | Melanoma | 6272 |
| RVIRPGCEL | HLA-C01:02 | PRDM7 | Melanoma | 6273 |
| TSPTRESL | HLA-C01:02 | PRDM7 | Melanoma | 6274 |
| MSPERSQEE | HLA-C01:02 | PRDM7 | Melanoma | 6275 |
| KIPMKNGHL | HLA-C01:02 | PRDM7 | Melanoma | 6276 |
| KGHPNRSAL | HLA-C01:02 | PRDM7 | Melanoma | 6277 |
| FSVNMWNAI | HLA-C03:04 | PRDM7 | Melanoma | 6278 |
| SSIEPAESL | HLA-C03:04 | PRDM7 | Melanoma | 6279 |
| ASENNESSL | HLA-C03:04 | PRDM7 | Melanoma | 6280 |
| KARDPSMSL | HLA-C03:04 | PRDM7 | Melanoma | 6281 |
| CAAHGPPTF | HLA-C03:04 | PRDM7 | Melanoma | 6282 |
| AANSGYSWL | HLA-C03:04 | PRDM7 | Melanoma | 6283 |
| ARDPSMSLM | HLA-C04:01 | PRDM7 | Melanoma | 6284 |
| RTSSLTAVL | HLA-C04:01 | PRDM7 | Melanoma | 6285 |
| AFKDISIYF | HLA-C04:01 | PRDM7 | Melanoma | 6286 |
| KARDPSMSL | HLA-C04:01 | PRDM7 | Melanoma | 6287 |
| VWNEASDL | HLA-C04:01 | PRDM7 | Melanoma | 6288 |
| SFNNESSL | HLA-C04:01 | PRDM7 | Melanoma | 6289 |
| SGDEYGQEL | HLA-C04:01 | PRDM7 | Melanoma | 6290 |
| LYCEMCQNFF | HLA-C04:01 | PRDM7 | Melanoma | 6291 |
| LRATRPAFM | HLA-C07:01 | PRDM7 | Melanoma | 6292 |
| SSANWMRYV | HLA-C07:01 | PRDM7 | Melanoma | 6293 |
| ARDPSMSLM | HLA-C07:01 | PRDM7 | Melanoma | 6294 |
| IRIRSGNIL | HLA-C07:01 | PRDM7 | Melanoma | 6295 |
| GRNCYEYVD | HLA-C07:01 | PRDM7 | Melanoma | 6296 |
| HRQIFYRTC | HLA-C07:01 | PRDM7 | Melanoma | 6297 |
| ARDPSMSL | HLA-C07:01 | PRDM7 | Melanoma | 6298 |
| ARDDEEQNL | HLA-C07:01 | PRDM7 | Melanoma | 6299 |
| LRATRPAFM | HLA-C07:02 | PRDM7 | Melanoma | 6300 |
| ARDPSMSLM | HLA-C07:02 | PRDM7 | Melanoma | 6301 |
| IRIRSGNIL | HLA-C07:02 | PRDM7 | Melanoma | 6302 |
| FQYHRQIFY | HLA-C07:02 | PRDM7 | Melanoma | 6303 |
| ARDPSMSL | HLA-C07:02 | PRDM7 | Melanoma | 6304 |
| IRPGCELL | HLA-C07:02 | PRDM7 | Melanoma | 6305 |
| VKPPWMAF | HLA-C07:02 | PRDM7 | Melanoma | 6306 |
| ARDDEEQNL | HLA-C07:02 | PRDM7 | Melanoma | 6307 |
| CRSQSRSRYY | HLA-A01:01 | PRM1 | Testis | 6308 |
| RSQSRSRYY | HLA-A01:01 | PRM1 | Testis | 6309 |
| CCRSQSRSRYY | HLA-A01:01 | PRM1 | Testis | 6310 |
| RSQSRSRY | HLA-A01:01 | PRM1 | Testis | 6311 |
| SQSRSRYY | HLA-A01:01 | PRM1 | Testis | 6312 |
| RAMRCCRPRY | HLA-A02:01 | PRM1 | Testis | 6313 |
| RAMRCCRPR | HLA-A02:01 | PRM1 | Testis | 6314 |
| AMRCCRPRY | HLA-A02:01 | PRM1 | Testis | 6315 |
| AMRCCRPRYR | HLA-A02:01 | PRM1 | Testis | 6316 |
| SQSRSRYYR | HLA-A02:01 | PRM1 | Testis | 6317 |
| RRAMRCCRPR | HLA-A02:01 | PRM1 | Testis | 6318 |
| RSQSRSRYYR | HLA-A02:01 | PRM1 | Testis | 6319 |
| CQTRRRAMRQ | HLA-A02:01 | PRM1 | Testis | 6320 |
| RAMRCCRPRYR | HLA-A02:01 | PRM1 | Testis | 6321 |
| MRCCRPRYR | HLA-A02:01 | PRM1 | Testis | 6322 |
| SCQTRRRAM | HLA-A02:01 | PRM1 | Testis | 6323 |
| RSCQTRRRA | HLA-A02:01 | PRM1 | Testis | 6324 |
| SRYYRQRQR | HLA-A02:01 | PRM1 | Testis | 6325 |
| YRCCRSQSR | HLA-A02:01 | PRM1 | Testis | 6326 |
| RYYRQRQRS | HLA-A02:01 | PRM1 | Testis | 6327 |
| RSQSRSRYY | HLA-A02:01 | PRM1 | Testis | 6328 |
| SCQTRRRA | HLA-A02:01 | PRM1 | Testis | 6329 |
| RAMRCCRPRY | HLA-A03:01 | PRM1 | Testis | 6330 |
| AMRCCRPRYR | HLA-A03:01 | PRM1 | Testis | 6331 |
| AMRCCRPRY | HLA-A03:01 | PRM1 | Testis | 6332 |
| RSQSRSRYY | HLA-A03:01 | PRM1 | Testis | 6333 |
| SRYYRQRQR | HLA-A03:01 | PRM1 | Testis | 6334 |
| RQRSRRRRR | HLA-A03:01 | PRM1 | Testis | 6335 |
| SQSRSRYYR | HLA-A11:01 | PRM1 | Testis | 6336 |
| RSQSRSRYYR | HLA-A11:01 | PRM1 | Testis | 6337 |
| RAMRCCRPR | HLA-A11:01 | PRM1 | Testis | 6338 |
| SRYYRQRQR | HLA-A11:01 | PRM1 | Testis | 6339 |
| RYYRQRQRS | HLA-A24:02 | PRM1 | Testis | 6340 |
| RYRCCRSQSR | HLA-A24:02 | PRM1 | Testis | 6341 |
| RYYRQRQRSR | HLA-A24:02 | PRM1 | Testis | 6342 |
| RYRPRCRRH | HLA-A24:02 | PRM1 | Testis | 6343 |
| YYRQRQRSR | HLA-A24:02 | PRM1 | Testis | 6344 |
| RYRCCRSQS | HLA-A30:01 | PRM1 | Testis | 6345 |
| RSRYYRQRQ | HLA-A30:01 | PRM1 | Testis | 6346 |
| RSRRRRRRS | HLA-A30:01 | PRM1 | Testis | 6347 |
| RSQSRSRYY | HLA-A30:01 | PRM1 | Testis | 6348 |
| RQRSRRRRR | HLA-A30:01 | PRM1 | Testis | 6349 |
| RSRYYRQR | HLA-A30:01 | PRM1 | Testis | 6350 |
| SQSRSRYYR | HLA-A33:03 | PRM1 | Testis | 6351 |
| RSQSRSRYYR | HLA-A33:03 | PRM1 | Testis | 6352 |
| AMRCCRPRYR | HLA-A33:03 | PRM1 | Testis | 6353 |
| RAMRCCRPR | HLA-A33:03 | PRM1 | Testis | 6354 |
| YYRQRQRSR | HLA-A33:03 | PRM1 | Testis | 6355 |
| SRYYRQRQR | HLA-A33:03 | PRM1 | Testis | 6356 |
| RYYRQRQR | HLA-A33:03 | PRM1 | Testis | 6357 |
| RSCQTRRRAM | HLA-B07:02 | PRM1 | Testis | 6358 |
| RPRYRPRC | HLA-B07:02 | PRM1 | Testis | 6359 |
| SCQTRRRAM | HLA-B07:02 | PRM1 | Testis | 6360 |
| RPRYRPRCR | HLA-B07:02 | PRM1 | Testis | 6361 |
| RPRYRPRCRR | HLA-B07:02 | PRM1 | Testis | 6362 |
| CQTRRRAM | HLA-B08:01 | PRM1 | Testis | 6363 |
| SCQTRRRAM | HLA-B08:01 | PRM1 | Testis | 6364 |
| MARYRCCRS | HLA-B08:01 | PRM1 | Testis | 6365 |
| SRRRRRRSC | HLA-B08:01 | PRM1 | Testis | 6366 |
| YYRQRQRSR | HLA-B08:01 | PRM1 | Testis | 6367 |
| RAMRCCRPRY | HLA-B13:02 | PRM1 | Testis | 6368 |
| AMRCCRPRY | HLA-B13:02 | PRM1 | Testis | 6369 |
| RAMRCCRPRYRP | HLA-B13:02 | PRM1 | Testis | 6370 |
| RYYRQRQRS | HLA-B13:02 | PRM1 | Testis | 6371 |
| RQRQRSRRR | HLA-B13:02 | PRM1 | Testis | 6372 |
| RQRSRRRRR | HLA-B13:02 | PRM1 | Testis | 6373 |
| RAMRCCRPRY | HLA-B46:01 | PRM1 | Testis | 6374 |
| RSQSRSRYY | HLA-B46:01 | PRM1 | Testis | 6375 |
| AMRCCRPRY | HLA-B46:01 | PRM1 | Testis | 6376 |
| RSQSRSRY | HLA-B46:01 | PRM1 | Testis | 6377 |
| RSQSRSRYY | HLA-C01:02 | PRM1 | Testis | 6378 |
| RSCQTRRRAM | HLA-C01:02 | PRM1 | Testis | 6379 |
| RAMRCCRPR | HLA-C01:02 | PRM1 | Testis | 6380 |
| AMRCCRPRY | HLA-C01:02 | PRM1 | Testis | 6381 |
| SCQTRRRAM | HLA-C01:02 | PRM1 | Testis | 6382 |
| RSQSRSRYY | HLA-C03:04 | PRM1 | Testis | 6383 |
| RAMRCCRPR | HLA-C03:04 | PRM1 | Testis | 6384 |
| RAMRCCRPRY | HLA-C03:04 | PRM1 | Testis | 6385 |
| SCQTRRRAM | HLA-C03:04 | PRM1 | Testis | 6386 |
| CQTRRRAM | HLA-C03:04 | PRM1 | Testis | 6387 |
| RAMRCCRPR | HLA-C04:01 | PRM1 | Testis | 6388 |
| SQSRSRYYR | HLA-C04:01 | PRM1 | Testis | 6389 |
| RSQSRSRYY | HLA-C04:01 | PRM1 | Testis | 6390 |
| RYRPRCRRH | HLA-C04:01 | PRM1 | Testis | 6391 |
| YYRQRQRS | HLA-C04:01 | PRM1 | Testis | 6392 |
| RYRPRCRR | HLA-C04:01 | PRM1 | Testis | 6393 |
| CRSQSRSRY | HLA-C07:01 | PRM1 | Testis | 6394 |
| CRSQSRSRYY | HLA-C07:01 | PRM1 | Testis | 6395 |
| RSQSRSRYY | HLA-C07:01 | PRM1 | Testis | 6396 |
| SRYYRQRQR | HLA-C07:01 | PRM1 | Testis | 6397 |
| CRSQSRSRY | HLA-C07:02 | PRM1 | Testis | 6398 |
| RSQSRSRYY | HLA-C07:02 | PRM1 | Testis | 6399 |
| YYRQRQRSR | HLA-C07:02 | PRM1 | Testis | 6400 |
| YRPRCRRH | HLA-C07:02 | PRM1 | Testis | 6401 |
| YRQRQRSR | HLA-C07:02 | PRM1 | Testis | 6402 |
| LSERSHEVY | HLA-A01:01 | PRM2 | Testis | 6403 |
| SLSERSHEVY | HLA-A01:01 | PRM2 | Testis | 6404 |
| LSPEHVEVY | HLA-A01:01 | PRM2 | Testis | 6405 |
| LGDPLNQNF | HLA-A01:01 | PRM2 | Testis | 6406 |
| SLSERSHEV | HLA-A02:01 | PRM2 | Testis | 6407 |
| GLSPEHVEV | HLA-A02:01 | PRM2 | Testis | 6408 |
| RSLSERSHEV | HLA-A02:01 | PRM2 | Testis | 6409 |
| QGLSPEHVEV | HLA-A02:01 | PRM2 | Testis | 6410 |
| KLPGPLTPSWKL | HLA-A02:01 | PRM2 | Testis | 6411 |
| SLGDPLNQNFL | HLA-A02:01 | PRM2 | Testis | 6412 |
| KLPGPLTPS | HLA-A02:01 | PRM2 | Testis | 6413 |
| QLSPEHVEVY | HLA-A02:01 | PRM2 | Testis | 6414 |
| SLSERSHEVY | HLA-A02:01 | PRM2 | Testis | 6415 |
| SLGDPLNQN | HLA-A02:01 | PRM2 | Testis | 6416 |
| FLSQKAAEP | HLA-A02:01 | PRM2 | Testis | 6417 |
| SLGDPLNQNF | HLA-A02:01 | PRM2 | Testis | 6418 |
| SLGDPLNQ | HLA-A02:01 | PRM2 | Testis | 6419 |
| KLPGPLTPSWK | HLA-A03:01 | PRM2 | Testis | 6420 |
| RTHGQSHYR | HLA-A03:01 | PRM2 | Testis | 6421 |
| LTPSWKLRK | HLA-A03:01 | PRM2 | Testis | 6422 |
| KLPGPLTPS | HLA-A03:01 | PRM2 | Testis | 6423 |
| RTHGQSHYR | HLA-A11:01 | PRM2 | Testis | 6424 |
| LTPSWKLRK | HLA-A11:01 | PRM2 | Testis | 6425 |
| RTHGQSHYRR | HLA-ALL:01 | PRM2 | Testis | 6426 |
| KLPGPLTPSW | HLA-A24:02 | PRM2 | Testis | 6427 |
| SLGDPLNQNF | HLA-A24:02 | PRM2 | Testis | 6428 |
| KLPGPLTPSWKL | HLA-A24:02 | PRM2 | Testis | 6429 |
| LGDPLNQNF | HLA-A24:02 | PRM2 | Testis | 6430 |
| KSRPKHQVR | HLA-A30:01 | PRM2 | Testis | 6431 |
| RYRVRSLSE | HLA-A30:01 | PRM2 | Testis | 6432 |
| RTRKRTCRR | HLA-A30:01 | PRM2 | Testis | 6433 |
| RSHEVYRQQ | HLA-A30:01 | PRM2 | Testis | 6434 |
| RSHEVYRQQL | HLA-A30:01 | PRM2 | Testis | 6435 |
| RTHGQSHYR | HLA-A33:03 | PRM2 | Testis | 6436 |
| HYRRRHCSR | HLA-A33:03 | PRM2 | Testis | 6437 |
| ERTHGQSHYR | HLA-A33:03 | PRM2 | Testis | 6438 |
| EHVEVYER | HLA-A33:03 | PRM2 | Testis | 6439 |
| EVYERTHGQ | HLA-A33:03 | PRM2 | Testis | 6440 |
| MVRYRVRSL | HLA-B07:02 | PRM2 | Testis | 6441 |
| GPLTPSWKI | HLA-B07:02 | PRM2 | Testis | 6442 |
| LPGPLTPSWKL | HLA-B07:02 | PRM2 | Testis | 6443 |
| LPGPLTPSW | HLA-B07:02 | PRM2 | Testis | 6444 |
| MVRYRVRSI | HLA-B08:01 | PRM2 | Testis | 6445 |
| MVRYRVRSLS | HLA-B08:01 | PRM2 | Testis | 6446 |
| VRYRVRSL | HLA-B08:01 | PRM2 | Testis | 6447 |
| CSRRRLHRI | HLA-B08:01 | PRM2 | Testis | 6448 |
| SLSERSHEV | HLA-B08:01 | PRM2 | Testis | 6449 |
| DPLNQNFL | HLA-B08:01 | PRM2 | Testis | 6450 |
| REHAEGTKL | HLA-B13:02 | PRM2 | Testis | 6451 |
| RSLSERSHEV | HLA-B13:02 | PRM2 | Testis | 6452 |
| CSRRRLHRI | HLA-B13:02 | PRM2 | Testis | 6453 |
| GLSPEHVEV | HLA-B13:02 | PRM2 | Testis | 6454 |
| NQNFLSQKA | HLA-B13:02 | PRM2 | Testis | 6455 |
| LSPEHVEVY | HLA-B46:01 | PRM2 | Testis | 6456 |
| MVRYRVRSL | HLA-B46:01 | PRM2 | Testis | 6457 |
| LSERSHEVY | HLA-B46:01 | PRM2 | Testis | 6458 |
| YERTHGQSH | HLA-B46:01 | PRM2 | Testis | 6459 |
| KAAEPGREH | HLA-B46:01 | PRM2 | Testis | 6460 |
| MVRYRVRSL | HLA-C01:02 | PRM2 | Testis | 6461 |
| SLSERSHEV | HLA-C01:02 | PRM2 | Testis | 6462 |
| LSPEHVEVY | HLA-C01:02 | PRM2 | Testis | 6463 |
| LSPEHVEV | HLA-C01:02 | PRM2 | Testis | 6464 |
| KLPGPLTPS | HLA-C01:02 | PRM2 | Testis | 6465 |
| MVRYRVRSL | HLA-C03:04 | PRM2 | Testis | 6466 |
| SLSERSHEV | HLA-C03:04 | PRM2 | Testis | 6467 |
| LSPEHVEVY | HLA-C03:04 | PRM2 | Testis | 6468 |
| KAAEPGREH | HLA-C03:04 | PRM2 | Testis | 6469 |
| AAEPGREH | HLA-C03:04 | PRM2 | Testis | 6470 |
| LGDPLNQNF | HLA-C04:01 | PRM2 | Testis | 6471 |
| RRHCSRRRL | HLA-C04:01 | PRM2 | Testis | 6472 |
| SLSERSHEV | HLA-C04:01 | PRM2 | Testis | 6473 |
| SHEVYRQQL | HLA-C04:01 | PRM2 | Testis | 6474 |
| LSPEHVEV | HLA-C04:01 | PRM2 | Testis | 6475 |
| LGDPLNQNFL | HLA-C04:01 | PRM2 | Testis | 6476 |
| RRHCSRRRL | HLA-C07:01 | PRM2 | Testis | 6477 |
| MVRYRVRSL | HLA-C07:01 | PRM2 | Testis | 6478 |
| RRRHRRESL | HLA-C07:01 | PRM2 | Testis | 6479 |
| VRYRVRSL | HLA-C07:01 | PRM2 | Testis | 6480 |
| LSPEHVEVY | HLA-C07:01 | PRM2 | Testis | 6481 |
| RRHCSRRRL | HLA-C07:02 | PRM2 | Testis | 6482 |
| MVRYRVRSL | HLA-C07:02 | PRM2 | Testis | 6483 |
| RRRHRRESL | HLA-C07:02 | PRM2 | Testis | 6484 |
| VRYRVRSL | HLA-C07:02 | PRM2 | Testis | 6485 |
| LSPEHVEVY | HLA-C07:02 | PRM2 | Testis | 6486 |
| FSFTYTPEY | HLA-A01:01 | RBPJL | Pancreas | 6487 |
| CSDWRWLRA | HLA-A01:01 | RBPJL | Pancreas | 6488 |
| ETGPTVCGY | HLA-A01:01 | RBPJL | Pancreas | 6489 |
| DVEAETMYRY | HLA-A01:01 | RBPJL | Pancreas | 6490 |
| ATDADALLE | HLA-A01:01 | RBPJL | Pancreas | 6491 |
| ATDADALL | HLA-A01:01 | RBPJL | Pancreas | 6492 |
| SLACTLEPV | HLA-A02:01 | RBPJL | Pancreas | 6493 |
| ALLDVDEPI | HLA-A02:01 | RBPJL | Pancreas | 6494 |
| YLSGPGWRV | HLA-A02:01 | RBPJL | Pancreas | 6495 |
| SLVCVVPDV | HLA-A02:01 | RBPJL | Pancreas | 6496 |
| YLSVEDGAFV | HLA-A02:01 | RBPJL | Pancreas | 6497 |
| FSTSLACTLEPV | HLA-A02:01 | RBPJL | Pancreas | 6498 |
| SLVQLVCTV | HLA-A02:01 | RBPJL | Pancreas | 6499 |
| TSLACTLEPV | HLA-A02:01 | RBPJL | Pancreas | 6500 |
| YLCLATEKV | HLA-A02:01 | RBPJL | Pancreas | 6501 |
| ALLESIHQE | HLA-A02:01 | RBPJL | Pancreas | 6502 |
| ALLDVDEPISQL | HLA-A02:01 | RBPJL | Pancreas | 6503 |
| SLQDRSEMQL | HLA-A02:01 | RBPJL | Pancreas | 6504 |
| GLLQRLALA | HLA-A02:01 | RBPJL | Pancreas | 6505 |
| GTFHSRLIK | HLA-A03:01 | RBPJL | Pancreas | 6506 |
| RILHAKVAQK | HLA-A03:01 | RBPJL | Pancreas | 6507 |
| KVISKPSQK | HLA-A03:01 | RBPJL | Pancreas | 6508 |
| ITLPPMIIRK | HLA-A03:01 | RBPJL | Pancreas | 6509 |
| TLPPMIIRK | HLA-A03:01 | RBPJL | Pancreas | 6510 |
| GTFHSRLIK | HLA-A11:01 | RBPJL | Pancreas | 6511 |
| QTVRILHAK | HLA-ALI:01 | RBPJL | Pancreas | 6512 |
| GTYLCLATEK | HLA-A11:01 | RBPJL | Pancreas | 6513 |
| TLPPMIIRK | HLA-A11:01 | RBPJL | Pancreas | 6514 |
| RSLPGTWTR | HLA-A11:01 | RBPJL | Pancreas | 6515 |
| ITLPPMIIR | HLA-A11:01 | RBPJL | Pancreas | 6516 |
| RYGSLVQLV | HLA-A24:02 | RBPJL | Pancreas | 6517 |
| LFYPSAFSF | HLA-A24:02 | RBPJL | Pancreas | 6518 |
| RWLRAPITI | HLA-A24:02 | RBPJL | Pancreas | 6519 |
| EFTRTNFHLF | HLA-A24:02 | RBPJL | Pancreas | 6520 |
| FYPSAFSF | HLA-A24:02 | RBPJL | Pancreas | 6521 |
| SVRPGHPGV | HLA-A30:01 | RBPJL | Pancreas | 6522 |
| GTFHSRLIK | HLA-A30:01 | RBPJL | Pancreas | 6523 |
| HSRLIKVISK | HLA-A30:01 | RBPJL | Pancreas | 6524 |
| KVISKPSQK | HLA-A30:01 | RBPJL | Pancreas | 6525 |
| FCSDWRWLR | HLA-A33:03 | RBPJL | Pancreas | 6526 |
| AFCSDWRWLR | HLA-A33:03 | RBPJL | Pancreas | 6527 |
| AAFCSDWRWLR | HLA-A33:03 | RBPJL | Pancreas | 6528 |
| ESIHQEFTR | HLA-A33:03 | RBPJL | Pancreas | 6529 |
| TYTPEYSVR | HLA-A33:03 | RBPJL | Pancreas | 6530 |
| ITLPPMIIR | HLA-A33:03 | RBPJL | Pancreas | 6531 |
| CPKEANRAL | HLA-B07:02 | RBPJL | Pancreas | 6532 |
| HEPGARRRAL | HLA-B07:02 | RBPIL | Pancreas | 6533 |
| KPSQKKQSL | HLA-B07:02 | RBPJL | Pancreas | 6534 |
| APITIPMSL | HLA-B07:02 | RBPAL | Pancreas | 6535 |
| SPPGGGGTYL | HLA-B07:02 | RBPJL | Pancreas | 6536 |
| TPVPLISTL | HLA-B07:02 | RBPJL | Pancreas | 6537 |
| EPGARRRAL | HLA-B08:01 | RBPJL | Pancreas | 6538 |
| MIRKVAKQCAL | HLA-B08:01 | RBPJL | Pancreas | 6539 |
| EGYVRYGSL | HLA-B08:01 | RBPJL | Pancreas | 6540 |
| KPSQKKQSL | HLA-B08:01 | RBPJL | Pancreas | 6541 |
| NPLTHLSL | HLA-B08:01 | RBPJL | Pancreas | 6542 |
| SDWRWLRAPI | HLA-B13:02 | RBPJL | Pancreas | 6543 |
| VEFSFSTSL | HLA-B13:02 | RBPJL | Pancreas | 6544 |
| RQWAAFTLHL | HLA-B13:02 | RBPJL | Pancreas | 6545 |
| RSSPEHTTI | HLA-B13:02 | RBPJL | Pancreas | 6546 |
| YLSGPGWRV | HLA-B13:02 | RBPJL | Pancreas | 6547 |
| RLIKVISKP | HLA-B13:02 | RBPJL | Pancreas | 6548 |
| FSFTYTPEY | HLA-B46:01 | RBPJL | Pancreas | 6549 |
| CVVPDVAAF | HLA-B46:01 | RBPJL | Pancreas | 6550 |
| FTRTNFHLF | HLA-B46:01 | RBPJL | Pancreas | 6551 |
| SARQWAAF | HLA-B46:01 | RBPJL | Pancreas | 6552 |
| PPREGYVRY | HLA-B46:01 | RBPJL | Pancreas | 6553 |
| FSTSLACTL | HLA-C01:02 | RBPJL | Pancreas | 6554 |
| SSPEHTTIL | HLA-C01:02 | RBPJL | Pancreas | 6555 |
| TMYRSPRSL | HLA-C01:02 | RBPJL | Pancreas | 6556 |
| RAPITIPMSL | HLA-C01:02 | RBPJL | Pancreas | 6557 |
| VTPVPLISTL | HLA-C01:02 | RBPJL | Pancreas | 6558 |
| FSTSLACTL | HLA-C03:04 | RBPJL | Pancreas | 6559 |
| LSLQDRSEM | HLA-C03:04 | RBPJL | Pancreas | 6560 |
| LVLRGGREL | HLA-C03:04 | RBPJL | Pancreas | 6561 |
| WAAFTLHL | HLA-C03:04 | RBPJL | Pancreas | 6562 |
| LFYPSAFSF | HLA-C04:01 | RBPJL | Pancreas | 6563 |
| FYPSAFSFTY | HLA-C04:01 | RBPJL | Pancreas | 6564 |
| MYRSPRSLV | HLA-C04:01 | RBPJL | Pancreas | 6565 |
| FYPSAFSF | HLA-C04:01 | RBPJL | Pancreas | 6566 |
| ATDADALL | HLA-C04:01 | RBPJL | Pancreas | 6567 |
| TRTNFHLFI | HLA-C07:01 | RBPJL | Pancreas | 6568 |
| VRYGSLVQL | HLA-C07:01 | RBPJL | Pancreas | 6569 |
| LRAPITIPM | HLA-C07:01 | RBPJL | Pancreas | 6570 |
| KRKHFRLVL | HLA-C07:01 | RBPJL | Pancreas | 6571 |
| LRGGVRRCL | HLA-C07:01 | RBPJL | Pancreas | 6572 |
| YRSPRSLVC | HLA-C07:01 | RBPJL | Pancreas | 6573 |
| TRTNFHLFI | HLA-C07:02 | RBPJL | Pancreas | 6574 |
| LRAPITIPM | HLA-C07:02 | RBPJL | Pancreas | 6575 |
| LFYPSAPSF | HLA-C07:02 | RBPJL | Pancreas | 6576 |
| ARQWAAFTL | HLA-C07:02 | RBPJL | Pancreas | 6577 |
| FYPSAFSF | HLA-C07:02 | RBPJL | Pancreas | 6578 |
| FYPSAFSFTY | HLA-C07:02 | RBPJL | Pancreas | 6579 |
| DSNPSELKY | HLA-A01:01 | RLN1 | Prostate | 6580 |
| ADSNPSELKY | HLA-A01:01 | RLN1 | Prostate | 6581 |
| AADSNPSELKY | HLA-A01:01 | RLN1 | Prostate | 6582 |
| FLGALSKLY | HLA-A01:01 | RLN1 | Prostate | 6583 |
| CTKRSLAKY | HLA-A01:01 | RLN1 | Prostate | 6584 |
| EAADSNPSELKY | HLA-A01:01 | RLN1 | Prostate | 6585 |
| DSNLSFEEF | HLA-A01:01 | RLN1 | Prostate | 6586 |
| DTETIIMLEF | HLA-A01:01 | RLN1 | Prostate | 6587 |
| FTREFLGALS | HLA-A01:01 | RLN1 | Prostate | 6588 |
| LLEFCLLL | HLA-A01:01 | RLN1 | Prostate | 6589 |
| LLEFCLLLN | HLA-A01:01 | RLN1 | Prostate | 6590 |
| DTETIIML | HLA-A01:01 | RLN1 | Prostate | 6591 |
| AADSNPSEL | HLA-A01:01 | RLN1 | Prostate | 6592 |
| PSELKYLGL | HLA-A01:01 | RLN1 | Prostate | 6593 |
| LSERQPSLP | HLA-A01:01 | RLN1 | Prostate | 6594 |
| IMLEFIANL | HLA-A02:01 | RLN1 | Prostate | 6595 |
| FLFHLLEFCL | HLA-A02:01 | RLN1 | Prostate | 6596 |
| SLPELQQYV | HLA-A02:01 | RLN1 | Prostate | 6597 |
| HLLEFCLLL | HLA-A02:01 | RLN1 | Prostate | 6598 |
| IIMLEFIANL | HLA-A02:01 | RLN1 | Prostate | 6599 |
| FALLEPCLLL | HLA-A02:01 | RLN1 | Prostate | 6600 |
| FIANLPPEL | HLA-A02:01 | RLN1 | Prostate | 6601 |
| ALFEKCCLI | HLA-A02:01 | RLN1 | Prostate | 6602 |
| FLFHLLEFCLLL | HLA-A02:01 | RLN1 | Prostate | 6603 |
| FLFHLLEFCLL | HLA-A02:01 | RLN1 | Prostate | 6604 |
| FLFHLLEFC | HLA-A02:01 | RLN1 | Prostate | 6605 |
| LLLNQFSRAV | HLA-A02:01 | RLN1 | Prostate | 6606 |
| FIQTVSLGI | HLA-A02:01 | RLN1 | Prostate | 6607 |
| IMLEFIANLP | HLA-A02:01 | RLN1 | Prostate | 6608 |
| LFLFHLLEFCL | HLA-A02:01 | RLN1 | Prostate | 6609 |
| MLEFIANL | HLA-A02:01 | RLN1 | Prostate | 6610 |
| IIIMLEFIANL | HLA-A02:01 | RLN1 | Prostate | 6611 |
| HLLEFCLLLN | HLA-A02:01 | RLN1 | Prostate | 6612 |
| RLFLFHLLEFCL | HLA-A02:01 | RLN1 | Prostate | 6613 |
| LFLFHLLEFCLL | HLA-A02:01 | RLN1 | Prostate | 6614 |
| ALSERQPSL | HLA-A02:01 | RLN1 | Prostate | 6615 |
| LLLNQFSRA | HLA-A02:01 | RLN1 | Prostate | 6616 |
| PSLPELQQYV | HLA-A02:01 | RLN1 | Prostate | 6617 |
| YVALFEKCCLI | HLA-A02:01 | RLN1 | Prostate | 6618 |
| LLNQFSRAV | HLA-A02:01 | RLN1 | Prostate | 6619 |
| FINKDTETI | HLA-A02:01 | RLN1 | Prostate | 6620 |
| TIIMLEFIANL | HLA-A02:01 | RLN1 | Prostate | 6621 |
| KLCGRELVRA | HLA-A02:01 | RLN1 | Prostate | 6622 |
| VAGDFIQTV | HLA-A02:01 | RLN1 | Prostate | 6623 |
| ELQQYVPAL | HLA-A02:01 | RLN1 | Prostate | 6624 |
| HLLEFCLL | HLA-A02:01 | RLN1 | Prostate | 6625 |
| KWKDDVIKL | HLA-A02:01 | RLN1 | Prostate | 6626 |
| FLGALSKL | HLA-A02:01 | RLN1 | Prostate | 6627 |
| NLPPELKAA | HLA-A02:01 | RLN1 | Prostate | 6628 |
| ALFEKCCL | HLA-A02:01 | RLN1 | Prostate | 6629 |
| ALKDSNLSF | HLA-A02:01 | RLN1 | Prostate | 6630 |
| FLFHLLEF | HLA-A02:01 | RLN1 | Prostate | 6631 |
| RLFLFHLL | HLA-A02:01 | RLN1 | Prostate | 6632 |
| KLCGRELV | HLA-A02:01 | RLN1 | Prostate | 6633 |
| KLYHPSSTKI | HLA-A02:01 | RLN1 | Prostate | 6634 |
| YLGLDTHSQ | HLA-A02:01 | RLN1 | Prostate | 6635 |
| KLYHPSSTK | HLA-A03:01 | RLN1 | Prostate | 6636 |
| SKLYHPSSTK | HLA-A03:01 | RLN1 | Prostate | 6637 |
| KLYHPSSTKIQK | HLA-A03:01 | RLN1 | Prostate | 6638 |
| KLYHPSSTKI | HLA-A03:01 | RLN1 | Prostate | 6639 |
| ALSKLYEPSSTK | HLA-A03:01 | RLN1 | Prostate | 6640 |
| LSKLYHPSSTK | HLA-A03:01 | RLN1 | Prostate | 664 |
| AEIVPSFINK | HLA-A03:01 | RLN1 | Prostate | 6642 |
| AICGMSTWSK | HLA-A03:01 | RLN1 | Prostate | 6643 |
| FIANLPPELK | HLA-A03:01 | RLN1 | Prostate | 6644 |
| CLIGCTKRSLAK | HLA-A03:01 | RLN1 | Prostate | 6645 |
| EIVPSFINK | HLA-A03:01 | RLN1 | Prostate | 6646 |
| REFLGALSK | HLA-A03:01 | RLN1 | Prostate | 6647 |
| RLFLFHLLEF | HLA-A03:01 | RLN1 | Prostate | 6648 |
| RPYVALFEK | HLA-A03:01 | RLN1 | Prostate | 6649 |
| HLLEFCLLI | HLA-A03:01 | RLN1 | Prostate | 6650 |
| RLFLFHLLE | HLA-A03:01 | RLN1 | Prostate | 6651 |
| AEIVPSFINK | HLA-A11:01 | RLN1 | Prostate | 6652 |
| AICGMSTWSK | HLA-A11:01 | RLN1 | Prostate | 6653 |
| KLYHPSSTK | HLA-A11:01 | RLN1 | Prostate | 6654 |
| EIVPSFINK | HLA-A11:01 | RLN1 | Prostate | 6655 |
| FIANLPPELK | HLA-A11:01 | RLN1 | Prostate | 6656 |
| SKLYHPSSTK | HLA-A11:01 | RLN1 | Prostate | 6657 |
| LQQYVPALK | HLA-A11:01 | RLN1 | Prostate | 6658 |
| IANLPPELK | HLA-A11:01 | RLN1 | Prostate | 6659 |
| AADSNPSELK | HLA-A11:01 | RLN1 | Prostate | 6660 |
| REFLGALSK | HLA-A11:01 | RLN1 | Prostate | 6661 |
| RPYVALFEK | HLA-A11:01 | RLN1 | Prostate | 6662 |
| SNLSFEEFKK | HLA-A11:01 | RLN1 | Prostate | 6663 |
| IVPSFINK | HLA-A11:01 | RLN1 | Prostate | 6664 |
| ADSNPSELK | HLA-A11:01 | RLN1 | Prostate | 6665 |
| SNLSFEEFK | HLA-A11:01 | RLN1 | Prostate | 6666 |
| RLFLFHLLER | HLA-A24:02 | RLN1 | Prostate | 6667 |
| LFLFHLLEF | HLA-A24:02 | RLN1 | Prostate | 6668 |
| LYHPSSTKI | HLA-A24:02 | RLN1 | Prostate | 6669 |
| PYVALFEKCCLI | HLA-A24:02 | RLN1 | Prostate | 6670 |
| SFINKDTETI | HLA-A24:02 | RLN1 | Prostate | 6671 |
| MPRLFLFHLLEF | HLA-A24:02 | RLN1 | Prostate | 6672 |
| REFLGALSKL | HLA-A24:02 | RLN1 | Prostate | 6673 |
| KLYHPSSTKI | HLA-A24:02 | RLN1 | Prostate | 6674 |
| KWKDDVIKL | HLA-A24:02 | RLN1 | Prostate | 6675 |
| PVAEIVPSF | HLA-A24:02 | RLN1 | Prostate | 6676 |
| EFCLLLNQF | HLA-A24:02 | RLN1 | Prostate | 6677 |
| ALKDSNLSP | HLA-A24:02 | RLN1 | Prostate | 6678 |
| IMLEFIANL | HLA-A24:02 | RLN1 | Prostate | 6679 |
| EFLGALSKL | HLA-A24:02 | RLN1 | Prostate | 6680 |
| KRRPYVALF | HLA-A24:02 | RLN1 | Prostate | 6681 |
| HSQKKRRPYV | HLA-A30:01 | RLN1 | Prostate | 6682 |
| SQKKRRPYV | HLA-A30:01 | RLN1 | Prostate | 6683 |
| KLYHPSSTK | HLA-A30:01 | RLN1 | Prostate | 6684 |
| LSKLYHPSS | HLA-A30:01 | RLN1 | Prostate | 6685 |
| FSRAVAAKWK | HLA-A30:01 | RLN1 | Prostate | 6686 |
| KALRTGSCFT | HLA-A30:01 | RLN1 | Prostate | 6687 |
| SQKKRRPYVA | HLA-A30:01 | RLN1 | Prostate | 6688 |
| LSKLYHPSSTK | HLA-A30:01 | RLN1 | Prostate | 6689 |
| RAQIAICGM | HLA-A30:01 | RLN1 | Prostate | 6690 |
| KWKDDVIKL | HLA-A30:01 | RLN1 | Prostate | 6691 |
| KCCLIGCTK | HLA-A30:01 | RLN1 | Prostate | 6692 |
| AEIVPSFINK | HLA-A30:01 | RLN1 | Prostate | 6693 |
| REFLGALSK | HLA-A30:01 | RLN1 | Prostate | 6694 |
| LSFEEFKKL | HLA-A30:01 | RLN1 | Prostate | 6695 |
| EIVPSFINK | HLA-A30:01 | RLN1 | Prostate | 6696 |
| HSQKKRRPY | HLA-A30:01 | RLN1 | Prostate | 6697 |
| ELKAALSER | HLA-A33:03 | RLN1 | Prostate | 6698 |
| EFKKLIRNR | HLA-A33:03 | RLN1 | Prostate | 6699 |
| CGMSTWSKR | HLA-A33:03 | RLN1 | Prostate | 6700 |
| EEFKKLIRNR | HLA-A33:03 | RLN1 | Prostate | 6701 |
| CLLLNQFSR | HLA-A33:03 | RLN1 | Prostate | 6702 |
| EFCLLLNQFSR | HLA-A33:03 | RLN1 | Prostate | 6703 |
| ALRTGSCFTR | HLA-A33:03 | RLN1 | Prostate | 6704 |
| PELKAALSER | HLA-A33:03 | RLN1 | Prostate | 6705 |
| FCLLLNQFSR | HLA-A33:03 | RLN1 | Prostate | 6706 |
| EIVPSFINK | HLA-A33:03 | RLN1 | Prostate | 6707 |
| DVIKLCGR | HLA-A33:03 | RLN1 | Prostate | 6708 |
| DTHSQKKRR | HLA-A33:03 | RLN1 | Prostate | 6709 |
| DTHSQKKR | HLA-A33:03 | RLN1 | Prostate | 6710 |
| DDVIKLCGR | HLA-A33:03 | RLN1 | Prostate | 6711 |
| TPRPVAGDF | HLA-B07:02 | RLN1 | Prostate | 6712 |
| APQTPRPVA | HLA-B07:02 | RLN1 | Prostate | 6713 |
| MPRLFLFHLL | HLA-B07:02 | RLN1 | Prostate | 6714 |
| VPALKDSNL | HLA-B07:02 | RLN1 | Prostate | 6715 |
| RPVAEIVPSF | HLA-B07:02 | RLN1 | Prostate | 6716 |
| MPRLFLFHL | HLA-B07:02 | RLN1 | Prostate | 6717 |
| LPPELKAAL | HLA-B07:02 | RLN1 | Prostate | 6718 |
| MPRLFLFHLLEF | HLA-B07:02 | RLN1 | Prostate | 6719 |
| TPRPVAEIV | HLA-B07:02 | RLN1 | Prostate | 6720 |
| HPSSTKIQKL | HLA-B07:02 | RLN1 | Prostate | 6721 |
| SPDGGKAL | HLA-B07:02 | RLN1 | Prostate | 6722 |
| MPRLFLFHLL | HLA-B08:01 | RLN1 | Prostate | 6723 |
| MPRLFLFHL | HLA-B08:01 | RLN1 | Prostate | 6724 |
| QKKRRPYVAL | HLA-B08:01 | RLN1 | Prostate | 6725 |
| SQKKRRPYVAL | HLA-B08:01 | RLN1 | Prostate | 6726 |
| SQKKRRPYV | HLA-B08:01 | RLN1 | Prostate | 6727 |
| HSQKKRRPYVAL | HLA-B08:01 | RLN1 | Prostate | 6728 |
| ELQQYVPAL | HLA-B08:01 | RLN1 | Prostate | 6729 |
| MPRLFLFHLLEF | HLA-B08:01 | RLN1 | Prostate | 6730 |
| LIRNRQSEA | HLA-B08:01 | RLN1 | Prostate | 6731 |
| DFIQTVSL | HLA-B08:01 | RLN1 | Prostate | 6732 |
| IGCTKRSL | HLA-B08:01 | RLN1 | Prostate | 6733 |
| VIKLCGREL | HLA-B08:01 | RLN1 | Prostate | 6734 |
| ALFEKCCL | HLA-B08:01 | RLN1 | Prostate | 6735 |
| STWSKRSL | HLA-B08:01 | RLN1 | Prostate | 6736 |
| HLLEFCLL | HLA-B08:01 | RLN1 | Prostate | 6737 |
| PPELKAAL | HLA-B08:01 | RLN1 | Prostate | 6738 |
| ALSERQPSL | HLA-B08:01 | RLN1 | Prostate | 6739 |
| LPPELKAAL | HLA-B08:01 | RLN1 | Prostate | 6740 |
| REFLGALSKL | HLA-B13:02 | RLN1 | Prostate | 6741 |
| RELVRAQIAI | HLA-B13:02 | RLN1 | Prostate | 6742 |
| LEFCLLLNQF | HLA-B13:02 | RLN1 | Prostate | 6743 |
| LEFIANLPP | HLA-B13:02 | RLN1 | Prostate | 6744 |
| GDFIQTVSL | HLA-B13:02 | RLN1 | Prostate | 6745 |
| LEFIANLPPEL | HLA-B13:02 | RLN1 | Prostate | 6746 |
| GDFIQTVSLGI | HLA-B13:02 | RLN1 | Prostate | 6747 |
| MLEFIANLPP | HLA-B13:02 | RLN1 | Prostate | 6748 |
| TETIIMLEFI | HLA-B13:02 | RLN1 | Prostate | 6749 |
| VAGDFIQTV | HLA-B13:02 | RLN1 | Prostate | 6750 |
| ALFEKCCLI | HLA-B13:02 | RLN1 | Prostate | 6751 |
| SLPELQQYV | HLA-B13:02 | RLN1 | Prostate | 6752 |
| QQYVPALKD | HLA-B13:02 | RLN1 | Prostate | 6753 |
| HLLEFCLLL | HLA-B13:02 | RLN1 | Prostate | 6754 |
| RQPSLPELQ | HLA-B13:02 | RLN1 | Prostate | 6755 |
| RLFLFHLLE | HLA-B13:02 | RLN1 | Prostate | 6756 |
| AGDFIQTV | HLA-B13:02 | RLN1 | Prostate | 6757 |
| RLFLFHLL | HLA-B13:02 | RLN1 | Prostate | 6758 |
| FTREFLGAL | HLA-B46:01 | RLN1 | Prostate | 6759 |
| FIANLPPEL | HLA-B46:01 | RLN1 | Prostate | 6760 |
| IAICGMSTW | HLA-B46:01 | RLN1 | Prostate | 6761 |
| FINKDTETI | HLA-B46:01 | RLN1 | Prostate | 6762 |
| FSRAVAAKW | HLA-B46:01 | RLN1 | Prostate | 6763 |
| FLGALSKLY | HLA-B46:01 | RLN1 | Prostate | 6764 |
| ALKDSNLSF | HLA-B46:01 | RLN1 | Prostate | 6765 |
| KALRTGSCF | HLA-B46:01 | RLN1 | Prostate | 6766 |
| CTKRSLAKY | HLA-B46:01 | RLN1 | Prostate | 6767 |
| ISPDGGKAL | HLA-B46:01 | RLN1 | Prostate | 6768 |
| VAGDFIQTV | HLA-B46:01 | RLN1 | Prostate | 6769 |
| VIKLCGREL | HLA-B46:01 | RLN1 | Prostate | 6770 |
| PVAEIVPSF | HLA-B46:01 | RLN1 | Prostate | 6771 |
| SLPELQQY | HLA-B46:01 | RLN1 | Prostate | 6772 |
| TIIIMLEF | HLA-B46:01 | RLN1 | Prostate | 6773 |
| FLFHLLEF | HLA-B46:01 | RLN1 | Prostate | 6774 |
| FIANLPPEL | HLA-C01:02 | RLN1 | Prostate | 6775 |
| ISPDGGKAL | HLA-C01:02 | RLN1 | Prostate | 6776 |
| FTREFLGAL | HLA-C01:02 | RLN1 | Prostate | 6777 |
| IMLEFIANL | HLA-C01:02 | RLN1 | Prostate | 6778 |
| MSTWSKRSL | HLA-C01:02 | RLN1 | Prostate | 6779 |
| SLPELQQYV | HLA-C01:02 | RLN1 | Prostate | 6780 |
| AADSNPSEL | HLA-C01:02 | RLN1 | Prostate | 6781 |
| ALSERQPSL | HLA-C01:02 | RLN1 | Prostate | 6782 |
| FIQTVSLGI | HLA-C01:02 | RLN1 | Prostate | 6783 |
| NLPPELKAAL | HLA-C01:02 | RLN1 | Prostate | 6784 |
| LPPELKAAL | HLA-C01:02 | RLN1 | Prostate | 6785 |
| SNPSELKYL | HLA-C01:02 | RLN1 | Prostate | 6786 |
| RQPSLPEL | HLA-C01:02 | RLN1 | Prostate | 6787 |
| DAPQTPRPV | HLA-C01:02 | RLN1 | Prostate | 6788 |
| FIANLPPEL | HLA-C03:04 | RLN1 | Prostate | 6789 |
| FTREFLGAL | HLA-C03:04 | RLN1 | Prostate | 6790 |
| MSTWSKRSL | HLA-C03:04 | RLN1 | Prostate | 6791 |
| AADSNPSEL | HLA-C03:04 | RLN1 | Prostate | 6792 |
| FINKDTETI | HLA-C03:04 | RLN1 | Prostate | 6793 |
| LAICGMSTW | HLA-C03:04 | RLN1 | Prostate | 6794 |
| RAQIAICQM | HLA-C03:04 | RLN1 | Prostate | 6795 |
| ISPDGGKAL | HLA-C03:04 | RLN1 | Prostate | 6796 |
| LSFEEFKKL | HLA-C03:04 | RLN1 | Prostate | 6797 |
| IANLPPEL | HLA-C03:04 | RLN1 | Prostate | 6798 |
| EAADSNPSEL | HLA-C03:04 | RLN1 | Prostate | 6799 |
| FIANLPPEL | HLA-C04:01 | RLN1 | Prostate | 6800 |
| AADSNPSEL | HLA-C04:01 | RLN1 | Prostate | 6801 |
| SLPELQQYV | HLA-C04:01 | RLN1 | Prostate | 6802 |
| LYHPSSTKI | HLA-C04:01 | RLN1 | Prostate | 6803 |
| FIQTVSLGI | HLA-C04:01 | RLN1 | Prostate | 6804 |
| KRRPYVALF | HLA-C04:01 | RLN1 | Prostate | 6805 |
| SFEEFKKLI | HLA-C04:01 | RLN1 | Prostate | 6806 |
| FHLLEFCLLL | HLA-C04:01 | RLN1 | Prostate | 6807 |
| FHLLEFCLL | HLA-C04:01 | RLN1 | Prostate | 6808 |
| SFEEFKKL | HLA-C04:01 | RLN1 | Prostate | 6809 |
| WKDDVIKL | HLA-C04:01 | RLN1 | Prostate | 6810 |
| LANLPPEL | HLA-C04:01 | RLN1 | Prostate | 6811 |
| LFHLLEFCL | HLA-C04:01 | RLN1 | Prostate | 6812 |
| LFLFHLLEF | HLA-C04:01 | RLN1 | Prostate | 6813 |
| EFCLLLNQF | HLA-C04:01 | RLN1 | Prostate | 6814 |
| DFIQTVSL | HLA-C04:01 | RLN1 | Prostate | 6815 |
| KRRPYVALF | HLA-C07:01 | RLN1 | Prostate | 6816 |
| ERQPSLPEL | HLA-C07:01 | RLN1 | Prostate | 6817 |
| LYHPSSTKI | HLA-C07:01 | RLN1 | Prostate | 6818 |
| KKRRPYVALF | HLA-C07:01 | RLN1 | Prostate | 6819 |
| VRAQIAICGM | HLA-C07:01 | RLN1 | Prostate | 6820 |
| FTREFLGAL | HLA-C07:01 | RLN1 | Prostate | 6821 |
| RRPYVALF | HLA-C07:01 | RLN1 | Prostate | 6822 |
| FIANLPPEL | HLA-C07:01 | RLN1 | Prostate | 6823 |
| MPRLFLFHLL | HLA-C07:01 | RLN1 | Prostate | 6824 |
| KRRPYVAL | HLA-C07:01 | RLN1 | Prostate | 6825 |
| GDFIQTVSL | HLA-C07:01 | RLN1 | Prostate | 6826 |
| TPRPVAGD | HLA-C07:01 | RLN1 | Prostate | 6827 |
| STWSKRSL | HLA-C07:01 | RLN1 | Prostate | 6828 |
| RRPYVALFE | HLA-C07:01 | RLN1 | Prostate | 6829 |
| KRSLSQED | HLA-C07:01 | RLN1 | Prostate | 6830 |
| LIGCTKRSL | HLA-C07:01 | RLN1 | Prostate | 6831 |
| KRRPYVALF | HLA-C07:02 | RLN1 | Prostate | 6832 |
| LYHPSSTKI | HLA-C07:02 | RLN1 | Prostate | 6833 |
| KKRRPYVALF | HLA-C07:02 | RLN1 | Prostate | 6834 |
| ERQPSLPEL | HLA-C07:02 | RLN1 | Prostate | 6835 |
| RRPYVALF | HLA-C07:02 | RLN1 | Prostate | 6836 |
| FHLLEFCLL | HLA-C07:02 | RLN1 | Prostate | 6837 |
| FTREFLGAL | HLA-C07:02 | RLN1 | Prostate | 6838 |
| FIANLPPEL | HLA-C07:02 | RLN1 | Prostate | 6839 |
| SQKKRRPYVALF | HLA-C07:02 | RLN1 | Prostate | 6840 |
| KRRPYVAL | HLA-C07:02 | RLN1 | Prostate | 6841 |
| ISPDGGKAL | HLA-C07:02 | RLN1 | Prostate | 6842 |
| KWKDDVIKL | HLA-C07:02 | RLN1 | Prostate | 6843 |
| KKRRPYVAL | HLA-C07:02 | RLN1 | Prostate | 6844 |
| IANLPPEL | HLA-C07:02 | RLN1 | Prostate | 6845 |
| FSEGAQHGPY | HLA-A01:01 | RSPH6A | Thyroid | 6846 |
| FTGYLDTPVVSY | HLA-A01:01 | RSPH6A | Thyroid | 6847 |
| FPSEFQPQPY | HLA-A01:01 | RSPH6A | Thyroid | 6848 |
| YLDTPVVSY | HLA-A01:01 | RSPH6A | Thyroid | 6849 |
| PSELGFPHY | HLA-A01:01 | RSPH6A | Thyroid | 6850 |
| YSDESRMQV | HLA-A01:01 | RSPH6A | Thyroid | 6851 |
| SLYEHLVNL | HLA-A02:01 | RSPH6A | Thyroid | 6852 |
| LLMPQVFQA | HLA-A02:01 | RSPH6A | Thyroid | 6853 |
| SLYEHLVNLL | HLA-A02:01 | RSPH6A | Thyroid | 6854 |
| GLPWTRLPHV | HLA-A02:01 | RSPH6A | Thyroid | 6855 |
| LSLYEHLVNL | HLA-A02:01 | RSPH6A | Thyroid | 6856 |
| HLAPWTTRL | HLA-A02:01 | RSPH6A | Thyroid | 6857 |
| TQWEWFHPKL | HLA-A02:01 | RSPH6A | Thyroid | 6858 |
| SMANWVHHT | HLA-A02:01 | RSPH6A | Thyroid | 6859 |
| NLWPGAYAYA | HLA-A02:01 | RSPH6A | Thyroid | 6860 |
| NLLMPQVFQA | HLA-A02:01 | RSPH6A | Thyroid | 6861 |
| SLCPQYSVAV | HLA-A02:01 | RSPH6A | Thyroid | 6862 |
| KADEGPEEV | HLA-A02:01 | RSPH6A | Thyroid | 6863 |
| YLDTPVVSY | HLA-A02:01 | RSPH6A | Thyroid | 6864 |
| ALQFLPSEL | HLA-A02:01 | RSPH6A | Thyroid | 6865 |
| RLGGMEYPSV | HLA-A02:01 | RSPH6A | Thyroid | 6866 |
| SLSQQENLL | HLA-A02:01 | RSPH6A | Thyroid | 6867 |
| GAYAYASGK | HLA-A03:01 | RSPH6A | Thyroid | 6868 |
| RCTWVNPLQK | HLA-A03:01 | RSPH6A | Thyroid | 6869 |
| SLYEHLVNLLTK | HLA-A03:01 | RSPH6A | Thyroid | 6870 |
| GAYAYASGKK | HLA-A03:01 | RSPH6A | Thyroid | 6871 |
| CTWVNPLQK | HLA-A03:01 | RSPH6A | Thyroid | 6872 |
| SVWKPPPVIPK | HLA-A03:01 | RSPH6A | Thyroid | 6873 |
| STRPCQPPFNK | HLA-A03:01 | RSPH6A | Thyroid | 6874 |
| VWKPPPVIPK | HLA-A03:01 | RSPH6A | Thyroid | 6875 |
| TTQWEWFHPK | HLA-A11:01 | RSPH6A | Thyroid | 6876 |
| TQWEWFHPK | HLA-A11:01 | RSPH6A | Thyroid | 6877 |
| GAYAYASGK | HLA-A11:01 | RSPH6A | Thyroid | 6878 |
| CTWVNPLQK | HLA-A11:01 | RSPH6A | Thyroid | 6879 |
| SVWKPPPVIPK | HLA-A11:01 | RSPH6A | Thyroid | 6880 |
| STRPCQPPENK | HLA-A11:01 | RSPH6A | Thyroid | 6881 |
| RTTQWEWFHPK | HLA-A11:01 | RSPH6A | Thyroid | 6882 |
| SYLVAEVEF | HLA-A24:02 | RSPH6A | Thyroid | 6883 |
| RSYLVAEVEF | HLA-A24:02 | RSPH6A | Thyroid | 6884 |
| MEYPSVNTGF | HLA-A24:02 | RSPH6A | Thyroid | 6885 |
| LFQQLDPTF | HLA-A24:02 | RSPH6A | Thyroid | 6886 |
| EYPSVNTGF | HLA-A24:02 | RSPH6A | Thyroid | 6887 |
| IYIGWGHKY | HLA-A24:02 | RSPH6A | Thyroid | 6888 |
| PYIRDDPALQF | HLA-A24:02 | RSPH6A | Thyroid | 6889 |
| KIKKFFTGYL | HLA-A30:01 | RSPH6A | Thyroid | 6890 |
| SFRIFLAMK | HLA-A30:01 | RSPH6A | Thyroid | 6891 |
| STRPCQPPF | HLA-A30:01 | RSPH6A | Thyroid | 6892 |
| GAYAYASGK | HLA-A30:01 | RSPH6A | Thyroid | 6893 |
| KIKKFFTGY | HLA-A30:01 | RSPH6A | Thyroid | 6894 |
| CTWVNPLQK | HLA-A30:01 | RSPH6A | Thyroid | 6895 |
| KFENIYIGW | HLA-A30:01 | RSPH6A | Thyroid | 6896 |
| YAYASGNLR | HLA-A33:03 | RSPH6A | Thyroid | 6897 |
| STAPRASTR | HLA-A33:03 | RSPH6A | Thyroid | 6898 |
| WFHPKLDTLR | HLA-A33:03 | RSPH6A | Thyroid | 6899 |
| HTQHILPQGR | HLA-A33:03 | RSPH6A | Thyroid | 6900 |
| QASQRRHSR | HLA-A33:03 | RSPH6A | Thyroid | 6901 |
| EQQPIHTCR | HLA-A33:03 | RSPH6A | Thyroid | 6902 |
| EKQKALFTR | HLA-A33:03 | RSPH6A | Thyroid | 6903 |
| MHLAPWTTR | HLA-A33:03 | RSPH6A | Thyroid | 6904 |
| APGWSQRGSL | HLA-B07:02 | RSPH6A | Thyroid | 6905 |
| APRASTRPC | HLA-B07:02 | RSPH6A | Thyroid | 6906 |
| RPEDPLSVL | HLA-B07:02 | RSPH6A | Thyroid | 6907 |
| APWTTRLSCSL | HLA-B07:02 | RSPH6A | Thyroid | 6908 |
| SPESFNPAL | HLA-B07:02 | RSPH6A | Thyroid | 6909 |
| YPSGPEIMEM | HLA-B07:02 | RSPH6A | Thyroid | 6910 |
| ILGIKRSYL | HLA-B08:01 | RSPH6A | Thyroid | 6911 |
| YLRAQIARISAA | HLA-B08:01 | RSPH6A | Thyroid | 6912 |
| YKMAEKQKAL | HLA-B08:01 | RSPH6A | Thyroid | 6913 |
| WCKILGIKRSYL | HLA-B08:01 | RSPH6A | Thyroid | 6914 |
| KMAEKQKAL | HLA-B08:01 | RSPH6A | Thyroid | 6915 |
| EGIPVLEL | HLA-B08:01 | RSPH6A | Thyroid | 6916 |
| DPLSVLESL | HLA-B08:01 | RSPH6A | Thyroid | 6917 |
| WFHPKLDTL | HLA-B08:01 | RSPH6A | Thyroid | 6918 |
| DIVPKSVW | HLA-B08:01 | RSPH6A | Thyroid | 6919 |
| CDLSLYEHLV | HLA-B13:02 | RSPH6A | Thyroid | 6920 |
| FEGIPVLELV | HLA-B13:02 | RSPH6A | Thyroid | 6921 |
| TQWEWFHPKL | HLA-B13:02 | RSPH6A | Thyroid | 6922 |
| CSLCPQYSV | HLA-B13:02 | RSPH6A | Thyroid | 6923 |
| AQIVNARKI | HLA-B13:02 | RSPH6A | Thyroid | 6924 |
| HLVNLLTKI | HLA-B13:02 | RSPH6A | Thyroid | 6925 |
| GQFNLYQTD | HLA-B13:02 | RSPH6A | Thyroid | 6926 |
| GQSSLFQQL | HLA-B13:02 | RSPH6A | Thyroid | 6927 |
| YAYASGKKF | HLA-B46:01 | RSPH6A | Thyroid | 6928 |
| LAVQNAKAY | HLA-B46:01 | RSPH6A | Thyroid | 6929 |
| SAATQVSPL | HLA-B46:01 | RSPH6A | Thyroid | 6930 |
| RSNLWPGAY | HLA-B46:01 | RSPH6A | Thyroid | 6931 |
| HSRDQAQAL | HLA-B46:01 | RSPH6A | Thyroid | 6932 |
| YLDTPVVSY | HLA-B46:01 | RSPH6A | Thyroid | 6933 |
| EARLGGMEY | HLA-B46:01 | RSPH6A | Thyroid | 6934 |
| NLWPGAYAY | HLA-B46:01 | RSPH6A | Thyroid | 6935 |
| SLYEHLVNL | HLA-C01:02 | RSPH6A | Thyroid | 6936 |
| YSPESENPAL | HLA-C01:02 | RSPH6A | Thyroid | 6937 |
| VGPPLLTPL | HLA-C01:02 | RSPH6A | Thyroid | 6938 |
| HLAPWITRL | HLA-C01:02 | RSPH6A | Thyroid | 6939 |
| ILPQGRCTW | HLA-C01:02 | RSPH6A | Thyroid | 6940 |
| NAPGWSQRGSL | HLA-C01:02 | RSPH6A | Thyroid | 6941 |
| YAYASGKKF | HLA-C03:04 | RSPH6A | Thyroid | 6942 |
| SAATQVSPL | HLA-C03:04 | RSPH6A | Thyroid | 6943 |
| FQEPPVNPL | HLA-C03:04 | RSPH6A | Thyroid | 6944 |
| TSINCDLSL | HLA-C03:04 | RSPH6A | Thyroid | 6945 |
| QVAELTTSL | HLA-C03:04 | RSPH6A | Thyroid | 6946 |
| YAYASGNL | HLA-C03:04 | RSPH6A | Thyroid | 6947 |
| FEGIPVLEL | HLA-C03:04 | RSPH6A | Thyroid | 6948 |
| IRDDPALQF | HLA-C04:01 | RSPH6A | Thyroid | 6949 |
| FQEPPVNPL | HLA-C04:01 | RSPH6A | Thyroid | 6950 |
| YSDESRMQV | HLA-C04:01 | RSPH6A | Thyroid | 6951 |
| LYEHLVNLL | HLA-C04:01 | RSPH6A | Thyroid | 6952 |
| SYLVAEVEF | HLA-C04:01 | RSPH6A | Thyroid | 6953 |
| LYEHLVNL | HLA-C04:01 | RSPH6A | Thyroid | 6954 |
| YLDTPVVSY | HLA-C04:01 | RSPH6A | Thyroid | 6955 |
| SRSGANKYL | HLA-C07:01 | RSPH6A | Thyroid | 6956 |
| FRIFLAMKQL | HLA-C07:01 | RSPH6A | Thyroid | 6957 |
| CRFWGKILGI | HLA-C07:01 | RSPH6A | Thyroid | 6958 |
| KRSYLVAEV | HLA-C07:01 | RSPH6A | Thyroid | 6959 |
| SRMQVAEL | HLA-C07:01 | RSPH6A | Thyroid | 6960 |
| PDFEGIPVL | HLA-C07:01 | RSPH6A | Thyroid | 6961 |
| PSGPEIMEM | HLA-C07:01 | RSPH6A | Thyroid | 6962 |
| QHGPYIRDD | HLA-C07:01 | RSPH6A | Thyroid | 6963 |
| WFHPKLDTL | HLA-C07:02 | RSPH6A | Thyroid | 6964 |
| IYIGWGHKY | HLA-C07:02 | RSPH6A | Thyroid | 6965 |
| YAYASGKKF | HLA-C07:02 | RSPH6A | Thyroid | 6966 |
| IRDDPALQP | HLA-C07:02 | RSPH6A | Thyroid | 6967 |
| VRSNLWPGAY | HLA-C07:02 | RSPH6A | Thyroid | 6968 |
| QRPEDPLSVL | HLA-C07:02 | RSPH6A | Thyroid | 6969 |
| ETLRLASSY | HLA-A01:01 | SCXB | Thyroid | 6970 |
| IETLRLASSY | HLA-A01:01 | SCXB | Thyroid | 6971 |
| RTNSVNTAF | HLA-A01:01 | SCXB | Thyroid | 6972 |
| PTEPADRKL | HLA-A01:01 | SCXB | Thyroid | 6973 |
| GSDSSGSDEK | HLA-A01:01 | SCXB | Thyroid | 6974 |
| YLYPEVSPL | HLA-A02:01 | SCXB | Thyroid | 6975 |
| RYLYPEVSPL | HLA-A02:01 | SCXB | Thyroid | 6976 |
| YLYPEVSPLS | HLA-A02:01 | SCXB | Thyroid | 6977 |
| RLASSYISHL | HLA-A02:01 | SCXB | Thyroid | 6978 |
| YISHLGNVLL | HLA-A02:01 | SCXB | Thyroid | 6979 |
| GRYLYPEVSPL | HLA-A02:01 | SCXB | Thyroid | 6980 |
| PGRYLYPEVSPL | HLA-A02:01 | SCXB | Thyroid | 6981 |
| MSFATLRPA | HLA-A02:01 | SCXB | Thyroid | 6982 |
| YLYPEVSPLSE | HLA-A02:01 | SCXB | Thyroid | 6983 |
| GSDEKPCRV | HLA-A02:01 | SCXB | Thyroid | 6984 |
| TLIPTEPAD | HLA-A02:01 | SCXB | Thyroid | 6985 |
| GLQGARRRA | HLA-A02:01 | SCXB | Thyroid | 6986 |
| YLYPEVSP | HLA-A02:01 | SCXB | Thyroid | 6987 |
| CLSNQRKLSK | HLA-A03:01 | SCXB | Thyroid | 6988 |
| RLASSYISH | HLA-A03:01 | SCXB | Thyroid | 6989 |
| LSNQRKLSK | HLA-A03:01 | SCXB | Thyroid | 6990 |
| YLYPEVSPL | HLA-A03:01 | SCXB | Thyroid | 6991 |
| TLRPAPPGRY | HLA-A03:01 | SCXB | Thyroid | 6992 |
| ATLRPAPPGR | HLA-A11:01 | SCXB | Thyroid | 6993 |
| RTLIPTEPADRK | HLA-A11:01 | SCXB | Thyroid | 6994 |
| LSNQRKLSK | HLA-A11:01 | SCXB | Thyroid | 6995 |
| CLSNQRKLSK | HLA-A11:01 | SCXB | Thyroid | 6996 |
| RLASSYISH | HLA-A11:01 | SCXB | Thyroid | 6997 |
| SDSSGSDEK | HLA-A11:01 | SCXB | Thyroid | 6998 |
| SYISHLGNV | HLA-A24:02 | SCXB | Thyroid | 6999 |
| SYISHLGNVL | HLA-A24:02 | SCXB | Thyroid | 7000 |
| RYLYPEVSPL | HLA-A24:02 | SCXB | Thyroid | 7001 |
| SYISHLGNVLL | HLA-A24:02 | SCXB | Thyroid | 7002 |
| LYPEVSPLS | HLA-A24:02 | SCXB | Thyroid | 7003 |
| RVHAARCGL | HLA-A30:01 | SCXB | Thyroid | 7004 |
| MSFATLRPA | HLA-A30:01 | SCXB | Thyroid | 7005 |
| RTLIPTEPA | HLA-A30:01 | SCXB | Thyroid | 7006 |
| KIETLRLASSYI | HLA-A30:01 | SCXB | Thyroid | 7007 |
| RPAPPGRYL | HLA-A30:01 | SCXB | Thyroid | 7008 |
| SYISHLGNV | HLA-A30:01 | SCXB | Thyroid | 7009 |
| HSGPAFFHAAR | HLA-A33:03 | SCXB | Thyroid | 7010 |
| SGPAFFHAAR | HLA-A33:03 | SCXB | Thyroid | 7011 |
| TLRPAPPGR | HLA-A33:03 | SCXB | Thyroid | 7012 |
| HTANARER | HLA-A33:03 | SCXB | Thyroid | 7013 |
| TANARERDR | HLA-A33:03 | SCXB | Thyroid | 7014 |
| ETLRLASSY | HLA-A33:03 | SCXB | Thyroid | 7015 |
| RPAPPGRYL | HLA-B07:02 | SCXB | Thyroid | 7016 |
| LRPAPPGRYL | HLA-B07:02 | SCXB | Thyroid | 7017 |
| IRPAPPGRYLY | HLA-B07:02 | SCXB | Thyroid | 7018 |
| QPCHSGPAF | HLA-B07:02 | SCXB | Thyroid | 7019 |
| EPRQRHTANA | HLA-B08:01 | SCXB | Thyroid | 7020 |
| YLYPEVSPL | HLA-B08:01 | SCXB | Thyroid | 7021 |
| EPRQRHTA | HLA-B08:01 | SCXB | Thyroid | 7022 |
| EPRQRHTAN | HLA-B08:01 | SCXB | Thyroid | 7023 |
| EPADRKLSK | HLA-B08:01 | SCXB | Thyroid | 7024 |
| QPKQICTF | HLA-B08:01 | SCXB | Thyroid | 7025 |
| MSFATLRPA | HLA-B13:02 | SCXB | Thyroid | 7026 |
| SSYISHLGNV | HLA-B13:02 | SCXB | Thyroid | 7027 |
| HSGPAFFHA | HLA-B13:02 | SCXB | Thyroid | 7028 |
| YLYPEVSPL | HLA-B13:02 | SCXB | Thyroid | 7029 |
| HLGNVLLA | HLA-B13:02 | SCXB | Thyroid | 7030 |
| RERDRTNSV | HLA-B13:02 | SCXB | Thyroid | 7031 |
| YLYPEVSPL | HLA-B46:01 | SCXB | Thyroid | 7032 |
| YISHLGNVL | HLA-B46:01 | SCXB | Thyroid | 7033 |
| RTNSVNTAF | HLA-B46:01 | SCXB | Thyroid | 7034 |
| RLASSYISH | HLA-B46:01 | SCXB | Thyroid | 7035 |
| YLYPEVSP | HLA-B46:01 | SCXB | Thyroid | 7036 |
| YLYPEVSPL | HLA-C01:02 | SCXB | Thyroid | 7037 |
| YISHLGNVL | HLA-C01:02 | SCXB | Thyroid | 7038 |
| ISHLGNVLL | HLA-C01:02 | SCXB | Thyroid | 7039 |
| LYPEVSPL | HLA-C01:02 | SCXB | Thyroid | 7040 |
| PAPPGRYL | HLA-C01:02 | SCXB | Thyroid | 7041 |
| YLYPEVSPL | HLA-C03:04 | SCXB | Thyroid | 7042 |
| LASSYISHL | HLA-C03:04 | SCXB | Thyroid | 7043 |
| YISHLGNVL | HLA-C03:04 | SCXB | Thyroid | 7044 |
| RPAPPGRYL | HLA-C04:01 | SCXB | Thyroid | 7045 |
| RTNSVNTAF | HLA-C04:01 | SCXB | Thyroid | 7046 |
| YLYPEVSPL | HLA-C04:01 | SCXB | Thyroid | 7047 |
| LYPEVSPL | HLA-C04:01 | SCXB | Thyroid | 7048 |
| ISHLGNVLL | HLA-C04:01 | SCXB | Thyroid | 7049 |
| LRPAPPGRYL | HLA-C07:01 | SCXB | Thyroid | 7050 |
| CRVHAARCGL | HLA-C07:01 | SCXB | Thyroid | 7051 |
| SYISHLGNV | HLA-C07:01 | SCXB | Thyroid | 7052 |
| LRPAPPGRY | HLA-C07:01 | SCXB | Thyroid | 7053 |
| RPAPPGRYL | HLA-C07:01 | SCXB | Thyroid | 7054 |
| YLYPEVSPL | HLA-C07:01 | SCXB | Thyroid | 7055 |
| RYLYPEVSP | HLA-C07:01 | SCXB | Thyroid | 7056 |
| SYISHLGNV | HLA-C07:02 | SCXB | Thyroid | 7057 |
| YLYPEVSPL | HLA-C07:02 | SCXB | Thyroid | 7058 |
| LRPAPPGRYL | HLA-C07:02 | SCXB | Thyroid | 7059 |
| LYPEVSPL | HLA-C07:02 | SCXB | Thyroid | 7060 |
| LRPAPPGRY | HLA-C07:02 | SCXB | Thyroid | 7061 |
| FTFNLANALY | HLA-A01:01 | SERPINI2 | Pancreas | 7062 |
| NTEFAVDLY | HLA-A01:01 | SERPINI2 | Pancreas | 7063 |
| KVDFKDVLY | HLA-A01:01 | SERPINI2 | Pancreas | 7064 |
| FSESSLNY | HLA-A01:01 | SERPINI2 | Pancreas | 7065 |
| FVLKSFFSA | HLA-A02:01 | SERPINI2 | Pancreas | 7066 |
| YLQEGFTV | HLA-A02:01 | SERPINI2 | Pancreas | 7067 |
| FIANHPFLFI | HLA-A02:01 | SERPINI2 | Pancreas | 7068 |
| FLWSLLLL | HLA-A02:01 | SERPINI2 | Pancreas | 7069 |
| ALYLQEGFTV | HLA-A02:01 | SERPINI2 | Pancreas | 7070 |
| FVLKSFFSAI | HLA-A02:01 | SERPINI2 | Pancreas | 7071 |
| VLYSLNITEI | HLA-A02:01 | SERPINI2 | Pancreas | 7072 |
| WLSEMQEEEV | HLA-A02:01 | SERPINI2 | Pancreas | 7073 |
| SLNYQVLEL | HLA-A02:01 | SERPINI2 | Pancreas | 7074 |
| FLWSLLLLF | HLA-A02:01 | SERPINI2 | Pancreas | 7075 |
| TLVLEMVQL | HLA-A02:01 | SERPINI2 | Pancreas | 7076 |
| YLQEGPTVK | HLA-A02:01 | SERPINI2 | Pancreas | 7077 |
| KSFFSAISEK | HLA-A03:01 | SERPINI2 | Pancreas | 7078 |
| KLITAQQILK | HLA-A03:01 | SERPINI2 | Pancreas | 7079 |
| AIYFKGDWK | HLA-A03:01 | SERPINI2 | Pancreas | 7080 |
| ALYLQEGFTVK | HLA-A03:01 | SERPINI2 | Pancreas | 7081 |
| STVKIPMMK | HLA-A11:01 | SERPINI2 | Pancreas | 7082 |
| KSFFSAISEK | HLA-A11:01 | SERPINI2 | Pancreas | 7083 |
| AIYFKGDWK | HLA-A11:01 | SERPINI2 | Pancreas | 7084 |
| TSAGEEFFVLK | HLA-A11:01 | SERPINI2 | Pancreas | 7085 |
| AGEEFFVLK | HLA-A11:01 | SERPINI2 | Pancreas | 7086 |
| QFIANHPFLF | HLA-A24:02 | SERPINI2 | Pancreas | 7087 |
| LYSLNITEIF | HLA-A24:02 | SERPINI2 | Pancreas | 7088 |
| LYLQEGFTV | HLA-A24:02 | SERPINI2 | Pancreas | 7089 |
| IYFKGDWKQKF | HLA-A24:02 | SERPINI2 | Pancreas | 7090 |
| QYLHGNKEF | HLA-A24:02 | SERPINI2 | Pancreas | 7091 |
| MMKALLRTK | HLA-A30:01 | SERPINI2 | Pancreas | 7092 |
| RTKYGYFSE | HLA-A30:01 | SERPINI2 | Pancreas | 7093 |
| KSFFSAISEK | HLA-A30:01 | SERPINI2 | Pancreas | 7094 |
| YFKGDWKQK | HLA-A30:01 | SERPINI2 | Pancreas | 7095 |
| SFFSAISEK | HLA-A30:01 | SERPINI2 | Pancreas | 7096 |
| EMISTWVER | HLA-A33:03 | SERPINI2 | Pancreas | 7097 |
| LFFGSQASR | HLA-A33:03 | SERPINI2 | Pancreas | 7098 |
| LLFFGSQASR | HLA-A33:03 | SERPINI2 | Pancreas | 7099 |
| DTQEIKGR | HLA-A33:03 | SERPINI2 | Pancreas | 7100 |
| ESILFMGR | HLA-A33:03 | SERPINI2 | Pancreas | 7101 |
| SPLGITLVL | HLA-B07:02 | SERPINI2 | Pancreas | 7102 |
| IPMMKALL | HLA-B07:02 | SERPINI2 | Pancreas | 7103 |
| FSPLGITLVL | HLA-B07:02 | SERPINI2 | Pancreas | 7104 |
| GPLTRLVL | HLA-B07:02 | SERPINI2 | Pancreas | 7105 |
| LLRTKYGYF | HLA-B08:01 | SERPINI2 | Pancreas | 7106 |
| VLKSFFSAI | HLA-B08:01 | SERPINI2 | Pancreas | 7107 |
| IPMMKALL | HLA-B08:01 | SERPINI2 | Pancreas | 7108 |
| DLYQEVSL | HLA-B08:01 | SERPINI2 | Pancreas | 7109 |
| AISEKKQEF | HLA-B08:01 | SERPINI2 | Pancreas | 7110 |
| HIPVIMSL | HLA-B08:01 | SERPINI2 | Pancreas | 7111 |
| KDVLYSLNI | HLA-B13:02 | SERPINI2 | Pancreas | 7112 |
| MDTIFLWSLLL | HLA-B13:02 | SERPINI2 | Pancreas | 7113 |
| MDTIFLWSLL | HLA-B13:02 | SERPINI2 | Pancreas | 7114 |
| YVSQVTQKV | HLA-B13:02 | SERPINI2 | Pancreas | 7115 |
| AQQQIRQTL | HLA-B13:02 | SERPINI2 | Pancreas | 7116 |
| AQKNTEFAV | HLA-B13:02 | SERPINI2 | Pancreas | 7117 |
| FTFNLANAL | HLA-B46:01 | SERPINI2 | Pancreas | 7118 |
| FIANHPFLF | HLA-B46:01 | SERPINI2 | Pancreas | 7119 |
| YSLNITEIF | HLA-B46:01 | SERPINI2 | Pancreas | 7120 |
| FTFNLANALY | HLA-B46:01 | SERPINI2 | Pancreas | 7121 |
| FAVDLYQEV | HLA-B46:01 | SERPINI2 | Pancreas | 7122 |
| TSAEKKQEF | HLA-B46:01 | SERPINI2 | Pancreas | 7123 |
| VTNPDTQEI | HLA-B46:01 | SERPINI2 | Pancreas | 7124 |
| FTFNLANAL | HLA-C01:02 | SERPINI2 | Pancreas | 7125 |
| FGPLTRLVL | HLA-C01:02 | SERPINI2 | Pancreas | 7126 |
| YGYFSESSL | HLA-C01:02 | SERPINI2 | Pancreas | 7127 |
| FSPLGITLVL | HLA-C01:02 | SERPINI2 | Pancreas | 7128 |
| FSPLGITL | HLA-C01:02 | SERPINI2 | Pancreas | 7129 |
| FTFNLANAL | HLA-C03:04 | SERPINI2 | Pancreas | 7130 |
| FAVDLYQEV | HLA-C03:04 | SERPINI2 | Pancreas | 7131 |
| YGYFSESSL | HLA-C03:04 | SERPINI2 | Pancreas | 7132 |
| IHIPVIMSL | HLA-C03:04 | SERPINI2 | Pancreas | 7133 |
| FIANHPFLF | HLA-C04:01 | SERPINI2 | Pancreas | 7134 |
| YFSESSLNY | HLA-C04:01 | SERPINI2 | Pancreas | 7135 |
| IFSPLGITL | HLA-C04:01 | SERPINI2 | Pancreas | 7136 |
| ITDSSEVYV | HLA-C04:01 | SERPINI2 | Pancreas | 7137 |
| TFNLANALYL | HLA-C04:01 | SERPINI2 | Pancreas | 7138 |
| FRKEDTQLI | HLA-C07:01 | SERPINI2 | Pancreas | 7139 |
| FIANHPFLF | HLA-C07:01 | SERPINI2 | Pancreas | 7140 |
| FTFNLANAL | HLA-C07:01 | SERPINI2 | Pancreas | 7141 |
| SAGEEFFVL | HLA-C07:01 | SERPINI2 | Pancreas | 7142 |
| SLNYQVLEL | HLA-C07:01 | SERPINI2 | Pancreas | 7143 |
| LNYQVLEL | HLA-C07:01 | SERPINI2 | Pancreas | 7144 |
| FRKEDTQLI | HLA-C07:02 | SERPINI2 | Pancreas | 7145 |
| FIANHPFLF | HLA-C07:02 | SERPINIZ | Pancreas | 7146 |
| IHIPVIMSL | HLA-C07:02 | SERPINI2 | Pancreas | 7147 |
| KKQEFTFNL | HLA-C07:02 | SERPINI2 | Pancreas | 7148 |
| SYKGDEFSL | HLA-C07:02 | SERPINI2 | Pancreas | 7149 |
| FRKEDTQL | HLA-C07:02 | SERPINI2 | Pancreas | 7150 |
| FTDFMGIVY | HLA-A01:01 | SLC45A2 | Melanoma | 7151 |
| FFTDFMGQIVY | HLA-A01:01 | SLC45A2 | Melanoma | 7152 |
| LSSDGMYEY | HLA-A01:01 | SLC45A2 | Melanoma | 7153 |
| LFFTDEMGQIVY | HLA-A01:01 | SLC45A2 | Melanoma | 7154 |
| YSAHNSTEFLIY | HLA-A01:01 | SLC45A2 | Melanoma | 7155 |
| FIDGPIKAY | HLA-A01:01 | SLC45A2 | Melanoma | 7156 |
| YTVPFNLITEY | HLA-A01:01 | SLC45A2 | Melanoma | 7157 |
| FLSPILGFL | HLA-A02:01 | SLC45A2 | Melanoma | 7158 |
| SLYSIVWFL | HLA-A02:01 | SLC45A2 | Melanoma | 7159 |
| FLSNMLFFT | HLA-A02:01 | SLC45A2 | Melanoma | 7160 |
| HLIGWTAFL | HLA-A02:01 | SLC45A2 | Melanoma | 7161 |
| SLYSYFQKV | HLA-A02:01 | SLC45A2 | Melanoma | 7162 |
| FLSPILGFLL | HLA-A02:01 | SLC45A2 | Melanoma | 7163 |
| VMSSTLYTV | HLA-A02:01 | SLC45A2 | Melanoma | 7164 |
| YLLGAIDWA | HLA-A02:01 | SLC45A2 | Melanoma | 7165 |
| LVLCSLFGV | HLA-A02:01 | SLC45A2 | Melanoma | 7166 |
| VMFFFSALV | HLA-A02:01 | SLC45A2 | Melanoma | 7167 |
| RLLGTEFQV | HLA-A02:01 | SLC45A2 | Melanoma | 7168 |
| SLADDGPPDSV | HLA-A02:01 | SLC45A2 | Melanoma | 7169 |
| GMYEYGSIEKV | HLA-A02:01 | SLC45A2 | Melanoma | 7170 |
| FLIYERGVEV | HLA-A02:01 | SLC45A2 | Melanoma | 7171 |
| TLYTVPFNL | HLA-A02:01 | SLC45A2 | Melanoma | 7172 |
| GMYEYGSIEK | HLA-A03:01 | SLC45A2 | Melanoma | 7173 |
| KVLVSYIGLK | HLA-A03:01 | SLC45A2 | Melanoma | 7174 |
| SVFSSLYSCK | HLA-A03:01 | SLC45A2 | Melanoma | 7175 |
| VLVSYIGLK | HLA-A03:01 | SLC45A2 | Melanoma | 7176 |
| ALIANPRRK | HLA-A03:01 | SLC45A2 | Melanoma | 7177 |
| SVFSSLYSY | HLA-A03:01 | SLC45A2 | Melanoma | 7178 |
| SSLYSYFQK | HLA-A11:01 | SLC45A2 | Melanoma | 7179 |
| SVFSSLYSCK | HLA-A11:01 | SLC45A2 | Melanoma | 7180 |
| SVFSSLYSYFQK | HLA-A11:01 | SLC45A2 | Melanoma | 7181 |
| SVFSSLYSY | HLA-A11:01 | SLC45A2 | Melanoma | 7182 |
| STEFLIYER | HLA-A11:01 | SLC45A2 | Melanoma | 7183 |
| RYLCISHLI | HLA-A24:02 | SLC45A2 | Melanoma | 7184 |
| LYFTGYLLF | HLA-A24:02 | SLC45A2 | Melanoma | 7185 |
| GLYFTGYLLF | HLA-A24:02 | SLC45A2 | Melanoma | 7186 |
| YRYLCISHLI | HLA-A24:02 | SLC45A2 | Melanoma | 7187 |
| VFSSLYSYF | HLA-A24:02 | SLC45A2 | Melanoma | 7188 |
| LYTVPFNLI | HLA-A24:02 | SLC45A2 | Melanoma | 7189 |
| SYIGLKGLYF | HLA-A24:02 | SLC45A2 | Melanoma | 7190 |
| GTRRAMTLK | HLA-A30:01 | SLC45A2 | Melanoma | 7191 |
| QTRRAMTLK | HLA-A30:01 | SLC45A2 | Melanoma | 7192 |
| RSRWGRRRPYI | HLA-A30:01 | SLC45A2 | Melanoma | 7193 |
| KVKNGYVNP | HLA-A30:01 | SLC45A2 | Melanoma | 7194 |
| GFIGLFPNV | HLA-A30:01 | SLC45A2 | Melanoma | 7195 |
| RSRWGRRRPY | HLA-A30:01 | SLC45A2 | Melanoma | 7196 |
| GQAGRHIYK | HLA-A30:01 | SLC45A2 | Melanoma | 7197 |
| DFMGQIVYR | HLA-A33:03 | SLC45A2 | Melanoma | 7198 |
| LVNMPPHYR | HLA-A33:03 | SLC45A2 | Melanoma | 7199 |
| TDFMGQIVYR | HLA-A33:03 | SLC45A2 | Melanoma | 7200 |
| STEFLIYER | HLA-A33:03 | SLC45A2 | Melanoma | 7201 |
| HYHALFTGTR | HLA-A33:03 | SLC45A2 | Melanoma | 7202 |
| RPYILTLGVM | HLA-B07:02 | SLC45A2 | Melanoma | 7203 |
| RPTSRLIMHSM | HLA-B07:02 | SLC45A2 | Melanoma | 7204 |
| FPNVYSTLVI | HLA-B07:02 | SLC45A2 | Melanoma | 7205 |
| RPTSRLIM | HLA-B07:02 | SLC45A2 | Melanoma | 7206 |
| QPPHWMDSL | HLA-B07:02 | SLC45A2 | Melanoma | 7207 |
| TPVLLSVGL | HLA-B07:02 | SLC45A2 | Melanoma | 7208 |
| GPDCVPRGSL | HLA-B07:02 | SLC45A2 | Melanoma | 7209 |
| WGRRRPYIL | HLA-B08:01 | SLC45A2 | Melanoma | 7210 |
| NPRRKLVWAI | HLA-B08:01 | SLC45A2 | Melanoma | 7211 |
| RLIMHSMAM | HLA-B08:01 | SLC45A2 | Melanoma | 7212 |
| TLKSLLRAL | HLA-B08:01 | SLC45A2 | Melanoma | 7213 |
| LIYERGVEV | HLA-B08:01 | SLC45A2 | Melanoma | 7214 |
| DGPIKAYL | HLA-B08:01 | SLC45A2 | Melanoma | 7215 |
| TGTRRAMTL | HLA-B08:01 | SLC45A2 | Melanoma | 7216 |
| YEYGSIEKV | HLA-B13:02 | SLC45A2 | Melanoma | 7217 |
| FDFAADFIDGPI | HLA-B13:02 | SLC45A2 | Melanoma | 7218 |
| FDFAADFI | HLA-B13:02 | SLC45A2 | Melanoma | 7219 |
| VEAAYVTPV | HLA-B13:02 | SLC45A2 | Melanoma | 7220 |
| SLYSYFQKV | HLA-B13:02 | SLC45A2 | Melanoma | 7221 |
| RLLGTEFQV | HLA-B13:02 | SLC45A2 | Melanoma | 7222 |
| SLYSIVWFL | HLA-B13:02 | SLC45A2 | Melanoma | 7223 |
| YSAHNSTEF | HLA-B46:01 | SLC45A2 | Melanoma | 7224 |
| FGVMSSTLY | HLA-B46:01 | SLC45A2 | Melanoma | 7225 |
| FLSPILGFL | HLA-B46:01 | SLC45A2 | Melanoma | 7226 |
| SVFSSLYSY | HLA-B46:01 | SLC45A2 | Melanoma | 7227 |
| FTDFMGQIVY | HLA-B46:01 | SLC45A2 | Melanoma | 7228 |
| SSKSFWSST | HLA-B46:01 | SLC45A2 | Melanoma | 7229 |
| FIDGPIKAY | HLA-B46:01 | SLC45A2 | Melanoma | 7230 |
| SMAMFGREF | HLA-B46:01 | SLC45A2 | Melanoma | 7231 |
| ISHLIGWTAF | HLA-B46:01 | SLC45A2 | Melanoma | 7232 |
| FLSPILGFL | HLA-C01:02 | SLC45A2 | Melanoma | 7233 |
| NMPPHYRYL | HLA-C01:02 | SLC45A2 | Melanoma | 7234 |
| SLPVQHAVL | HLA-C01:02 | SLC45A2 | Melanoma | 7235 |
| YSAHNSTEF | HLA-C01:02 | SLC45A2 | Melanoma | 7236 |
| LFPNVYSTL | HLA-C01:02 | SLC45A2 | Melanoma | 7237 |
| SLPVQHAV | HLA-C01:02 | SLC45A2 | Melanoma | 7238 |
| YSAHNSTEF | HLA-C03:04 | SLC45A2 | Melanoma | 7239 |
| HASSLPLPL | HLA-C03:04 | SLC45A2 | Melanoma | 7240 |
| LSVGLPSSL | HLA-C03:04 | SLC45A2 | Melanoma | 7241 |
| YSTLVLCSL | HLA-C03:04 | SLC45A2 | Melanoma | 7242 |
| GAIDWAHLEL | HLA-C03:04 | SLC45A2 | Melanoma | 7243 |
| AAYVTPVLL | HLA-C03:04 | SLC45A2 | Melanoma | 7244 |
| AAYVTPVL | HLA-C03:04 | SLC45A2 | Melanoma | 7245 |
| AIDWAHLEL | HLA-C03:04 | SLC45A2 | Melanoma | 7246 |
| LFDFAADFI | HLA-C04:01 | SLC45A2 | Melanoma | 7247 |
| LFPNVYSTI | HLA-C04:01 | SLC45A2 | Melanoma | 7248 |
| FLSPILGFL | HLA-C04:01 | SLC45A2 | Melanoma | 7249 |
| AIDWAHLEL | HLA-C04:01 | SLC45A2 | Melanoma | 7250 |
| LFDFAADF | HLA-C04:01 | SLC45A2 | Melanoma | 7251 |
| YRYLCISHL | HLA-C07:01 | SLC45A2 | Melanoma | 7252 |
| RRRPYILTL | HLA-C07:01 | SLC45A2 | Melanoma | 7253 |
| YRYLCISHLI | HLA-C07:01 | SLC45A2 | Melanoma | 7254 |
| RRAMTLKSL | HLA-C07:01 | SLC45A2 | Melanoma | 7255 |
| RRPYILTL | HLA-C07:01 | SLC45A2 | Melanoma | 7256 |
| TDFMGQIVY | HLA-C07:01 | SLC45A2 | Melanoma | 7257 |
| RRRPYILTL | HLA-C07:02 | SLC45A2 | Melanoma | 7258 |
| YRYLCISHL | HLA-C07:02 | SLC45A2 | Melanoma | 7259 |
| NMPPHYRYL | HLA-C07:02 | SLC45A2 | Melanoma | 7260 |
| RRAMTLKSL | HLA-C07:02 | SLC45A2 | Melanoma | 7261 |
| LYFTGYLLF | HLA-C07:02 | SLC45A2 | Melanoma | 7262 |
| KRPTSRLIM | HLA-C07:02 | SLC45A2 | Melanoma | 7263 |
| RRPYILTL | HLA-C07:02 | SLC45A2 | Melanoma | 7264 |
| MSGAQDNSCLY | HLA-A01:01 | SPATA8 | Testis | 7265 |
| GMSGAQDNSCLY | HLA-A01:01 | SPATA8 | Testis | 7266 |
| GAQDNSCLY | HLA-A01:01 | SPATA8 | Testis | 7267 |
| AQDNSCLY | HLA-A01:01 | SPATA8 | Testis | 7268 |
| DSEDKQEKC | HLA-A01:01 | SPATA8 | Testis | 7269 |
| PTDSEDKQ | HLA-A01:01 | SPATA8 | Testis | 7270 |
| GLKGPVWPA | HLA-A02:01 | SPATA8 | Testis | 7271 |
| CLYQEIAPS | HLA-A02:01 | SPATA8 | Testis | 7272 |
| GGLKGPVWPA | HLA-A02:01 | SPATA8 | Testis | 7273 |
| RVPSASPLI | HLA-A02:01 | SPATA8 | Testis | 7274 |
| SCLYQEIAPS | HLA-A02:01 | SPATA8 | Testis | 7275 |
| VLFHPYCWS | HLA-A02:01 | SPATA8 | Testis | 7276 |
| CLYQEIAPSF | HLA-A02:01 | SPATA8 | Testis | 7277 |
| CPVSPILLVL | HLA-A02:01 | SPATA8 | Testis | 7278 |
| GLKGPVWPAK | HLA-A02:01 | SPATA8 | Testis | 7279 |
| PVSPILLVL | HLA-A02:01 | SPATA8 | Testis | 7280 |
| EIAPSFQRL | HLA-A02:01 | SPATA8 | Testis | 7281 |
| SASPLIQKI | HLA-A02:01 | SPATA8 | Testis | 7282 |
| GVACKGREQL | HLA-A02:01 | SPATA8 | Testis | 7283 |
| CPVSPILLV | HLA-A02:01 | SPATA8 | Testis | 7284 |
| ILLVLIFQ | HLA-A02:01 | SPATA8 | Testis | 7285 |
| GLKGPVWPAK | HLA-A03:01 | SPATA8 | Testis | 7286 |
| AMTCPCGWRPFK | HLA-A03:01 | SPATA8 | Testis | 7287 |
| RVPSASPLIQK | HLA-A03:01 | SPATA8 | Testis | 7288 |
| PSASPLIQK | HLA-A03:01 | SPATA8 | Testis | 7289 |
| VPSASPLIQK | HLA-A11:01 | SPATA8 | Testis | 7290 |
| RVPSASPLIQK | HLA-A11:01 | SPATA8 | Testis | 7291 |
| MTCPCGWRPFK | HLA-A11:01 | SPATA8 | Testis | 7292 |
| PSASPLIQK | HLA-A11:01 | SPATA8 | Testis | 7293 |
| SASPLIQK | HLA-A11:01 | SPATA8 | Testis | 7294 |
| LYQEIAPSF | HLA-A24:02 | SPATA8 | Testis | 7295 |
| CLYQEIAPSF | HLA-A24:02 | SPATA8 | Testis | 7296 |
| SCLYQEIAPSF | HLA-A24:02 | SPATA8 | Testis | 7297 |
| KINRRSVLF | HLA-A24:02 | SPATA8 | Testis | 7298 |
| KCPVSPILL | HLA-A24:02 | SPATA8 | Testis | 7299 |
| RVQRRRVPSA | HLA-A30:01 | SPATA8 | Testis | 7300 |
| RVQRRRVPS | HLA-A30:01 | SPATA8 | Testis | 7301 |
| RTSSRHFSEA | HLA-A30:01 | SPATA8 | Testis | 7302 |
| PSASPLIQK | HLA-A30:01 | SPATA8 | Testis | 7303 |
| SRKDPKGSK | HLA-A30:01 | SPATA8 | Testis | 7304 |
| GLKGPVWPAK | HLA-A30:01 | SPATA8 | Testis | 7305 |
| SFQRLPCPR | HLA-A33:03 | SPATA8 | Testis | 7306 |
| EAMTCPCGWR | HLA-A33:03 | SPATA8 | Testis | 7307 |
| EAFQGWPRR | HLA-A33:03 | SPATA8 | Testis | 7308 |
| EIAPSFQR | HLA-A33:03 | SPATA8 | Testis | 7309 |
| EENSCSHGR | HLA-A33:03 | SPATA8 | Testis | 7310 |
| WPRRPQGPGV | HLA-B07:02 | SPATA8 | Testis | 7311 |
| RPFKGGPGGL | HLA-B07:02 | SPATA8 | Testis | 7312 |
| RPQGPGVAC | HLA-B07:02 | SPATA8 | Testis | 7313 |
| CPRTSSRHF | HLA-B07:02 | SPATA8 | Testis | 7314 |
| SPILLVLIF | HLA-B07:02 | SPATA8 | Testis | 7315 |
| VQRRRVPSA | HLA-B08:01 | SPATA8 | Testis | 7316 |
| IQKINRRSVL | HLA-B08:01 | SPATA8 | Testis | 7317 |
| LIQKINRRSVL | HLA-B08:01 | SPATA8 | Testis | 7318 |
| SPILLVLI | HLA-B08:01 | SPATA8 | Testis | 7319 |
| VSPILLVL | HLA-B08:01 | SPATA8 | Testis | 7320 |
| CPVSPILL | HLA-B08:01 | SPATA8 | Testis | 7321 |
| CPVSPILLV | HLA-B13:02 | SPATA8 | Testis | 7322 |
| QEIAPSFQRL | HLA-B13:02 | SPATA8 | Testis | 7323 |
| KQEKCPVSPI | HLA-B13:02 | SPATA8 | Testis | 7324 |
| ISASPLIQKI | HLA-B13:02 | SPATA8 | Testis | 7325 |
| CSHGRIQRV | HLA-B13:02 | SPATA8 | Testis | 7326 |
| ASPLIQKI | HLA-B13:02 | SPATA8 | Testis | 7327 |
| GAQDNSCLY | HLA-B46:01 | SPATA8 | Testis | 7328 |
| SSRHFSEAM | HLA-B46:01 | SPATA8 | Testis | 7329 |
| MTCPCGWRPF | HLA-B46:01 | SPATA8 | Testis | 7330 |
| PAKEENSCSH | HLA-B46:01 | SPATA8 | Testis | 7331 |
| SRKDPKGSK | HLA-B46:01 | SPATA8 | Testis | 7332 |
| RVPSASPLI | HLA-C01:02 | SPATA8 | Testis | 7333 |
| SSRHFSEAM | HLA-C01:02 | SPATA8 | Testis | 7334 |
| KINRRSVLF | HLA-C01:02 | SPATA8 | Testis | 7335 |
| LAPSFQRL | HLA-C01:02 | SPATA8 | Testis | 7336 |
| RVPSASPL | HLA-C01:02 | SPATA8 | Testis | 7337 |
| VSPILLVL | HLA-C01:02 | SPATA8 | Testis | 7338 |
| SSRHFSEAM | HLA-C03:04 | SPATA8 | Testis | 7339 |
| SASPLIQKI | HLA-C03:04 | SPATA8 | Testis | 7340 |
| RVPSASPLI | HLA-C03:04 | SPATA8 | Testis | 7341 |
| VSPILLVL | HLA-C03:04 | SPATA8 | Testis | 7342 |
| IAPSFQRL | HLA-C03:04 | SPATA8 | Testis | 7343 |
| PVSPILLVL | HLA-C03:04 | SPATA8 | Testis | 7344 |
| LYQEIAPSF | HLA-C04:01 | SPATA8 | Testis | 7345 |
| RVPSASPLI | HLA-C04:01 | SPATA8 | Testis | 7346 |
| KINRRSVLF | HLA-C04:01 | SPATA8 | Testis | 7347 |
| KCPVSPILL | HLA-C04:01 | SPATA8 | Testis | 7348 |
| VSPILLVLI | HLA-C04:01 | SPATA8 | Testis | 7349 |
| RRVPSASPL | HLA-C07:01 | SPATA8 | Testis | 7350 |
| RRVPSASPLI | HLA-C07:01 | SPATA8 | Testis | 7351 |
| CSHGRIQRV | HLA-C07:01 | SPATA8 | Testis | 7352 |
| RRSVLFHPY | HLA-C07:01 | SPATA8 | Testis | 7353 |
| RRPQGPGVA | HLA-C07:01 | SPATA8 | Testis | 7354 |
| LYQEIAPSF | HLA-C07:02 | SPATA8 | Testis | 7355 |
| RRVPSASPL | HLA-C07:02 | SPATA8 | Testis | 7356 |
| RRSVLFHPY | HLA-C07:02 | SPATA8 | Testis | 7357 |
| NRRSVLFHPY | HLA-C07:02 | SPATA8 | Testis | 7358 |
| YSEKISYVY | HLA-A01:01 | SSX1 | Liver; Melanoma; Thyroid | 2359 |
| KYSEKISYVY | HLA-A01:01 | SSX1 | Liver; Melanoma; Thyroid | 7360 |
| ATDFQGNDF | HLA-A01:01 | SSX1 | Liver; Melanoma; Thyroid | 7361 |
| ATDFQGNDFD | HLA-A01:01 | SSX1 | Liver; Melanoma; Thyroid | 7362 |
| AMTKLGFKV | HLA-A02:01 | SSX1 | Liver; Melanoma; Thyroid | 7363 |
| RLHRIPKI | HLA-A02:01 | SSX1 | Liver; Melanoma; Thyroid | 7364 |
| MTFGRLHRI | HLA-A02:01 | SSX1 | Liver; Melanoma; Thyroid | 7365 |
| KMKYSEKISYV | HLA-A02:01 | SSX1 | Liver; Melanoma; Thyroid | 7366 |
| KAMTKLGFKV | HLA-A02:01 | SSX1 | Liver; Melanoma; Thyroid | 7367 |
| QMTFGRLHRI | HLA-A02:01 | SSX1 | Liver; Melanoma; Thyroid | 7368 |
| YKAMTKLGFKV | HLA-A02:01 | SSX1 | Liver; Melanoma; Thyroid | 7369 |
| KQLVIYEEL | HLA-A02:01 | SSX1 | Liver; Melanoma; Thyroid | 7370 |
| KKMKYSEKISYV | HLA-A02:01 | SSX1 | Liver; Melanoma; Thyroid | 7371 |
| RLRERKQLV | HLA-A02:01 | SSX1 | Liver; Melanoma; Thyroid | 7372 |
| KYSEKISYV | HLA-A02:01 | SSX1 | Liver; Melanoma; Thyroid | 7373 |
| KLGFKVTL | HLA-A02:01 | SSX1 | Liver; Melanoma; Thyroid | 7374 |
| TKLGFKVTL | HLA-A02:01 | SSX1 | Liver; Melanoma; Thyroid | 7375 |
| RLRERKQLVI | HLA-A02:01 | SSX1 | Liver; Melanoma; Thyroid | 7376 |
| KLGFKVTLP | HLA-A02:01 | SSX1 | Liver; Melanoma; Thyroid | 7377 |
| KAFDDIATY | HLA-A02:01 | SSX1 | Liver; Melanoma; Thyroid | 7378 |
| RIIPKIMPK | HLA-A03:01 | SSX1 | Liver; Melanoma; Thyroid | 7379 |
| YVYMKRNYK | HLA-A03:01 | SSX1 | Liver; Melanoma; Thyroid | 7380 |
| RIIPKIMPKK | HLA-A03:01 | SSX1 | Liver; Melanoma; Thyroid | 7381 |
| VTLPPFMCNK | HLA-A03:01 | SSX1 | Liver; Melanoma; Thyroid | 7382 |
| RIPKIMPK | HLA-A11:01 | SSX1 | Liver; Melanoma; Thyroid | 7383 |
| MTFGRLHRIIPK | HLA-A11:01 | SSX1 | Liver; Melanoma; Thyroid | 7384 |
| VTLPPFMQNK | HLA-A11:01 | SSX1 | Liver; Melanoma; Thyroid | 7385 |
| ATYFSKKEWKK | HLA-A11:01 | SSX1 | Liver; Melanoma; Thyroid | 7386 |
| KYSEKISYV | HLA-A24:02 | SSX1 | Liver; Melanoma; Thyroid | 7387 |
| VYMKRNYKAM | HLA-A24:02 | SSX1 | Liver; Melanoma; Thyroid | 7388 |
| NYKAMTKLGF | HLA-A24:02 | SSX1 | Liver; Melanoma; Thyroid | 7389 |
| AFDDIATYF | HLA-A24:02 | SSX1 | Liver; Melanoma; Thyroid | 7390 |
| KAMTKLGFK | HLA-A30:01 | SSX1 | Liver; Melanoma; Thyroid | 7391 |
| RIIPKIMPK | HLA-A30:01 | SSX1 | Liver; Melanoma; Thyroid | 7392 |
| YVYMKRNYK | HLA-A30:01 | SSX1 | Liver; Melanoma; Thyroid | 7393 |
| RIIPKIMPKK | HLA-A30:01 | SSX1 | Liver; Melanoma; Thyroid | 7394 |
| KYSEKISYV | HLA-A30:01 | SSX1 | Liver; Melanoma; Thyroid | 7395 |
| MTFGRLHR | HLA-A33:03 | SSX1 | Liver; Melanoma; Thyroid | 7396 |
| YVYMKRNYK | HLA-A33:03 | SSX1 | Liver; Melanoma; Thyroid | 7397 |
| HAWTHRLRER | HLA-A33:03 | SSX1 | Liver; Melanoma; Thyroid | 7398 |
| NISEKINKR | HLA-A33:03 | SSX1 | Liver; Melanoma; Thyroid | 7399 |
| NDFDNDHNR | HLA-A33:03 | SSX1 | Liver; Melanoma; Thyroid | 7400 |
| HPQMTFGRL | HLA-B07:02 | SSX1 | Liver; Melanoma; Thyroid | 7401 |
| RPRDDAKAS | HLA-B07:02 | SSX1 | Liver; Melanoma; Thyroid | 7402 |
| GPQNDGKQL | HLA-B07:02 | SSX1 | Liver; Melanoma; Thyroid | 7403 |
| EHPQMTFGRL | HLA-B07:02 | SSX1 | Liver; Melanoma; Thyroid | 7404 |
| YMKRNYKAM | HLA-B08:01 | SSX1 | Liver; Melanoma; Thyroid | 7405 |
| VYMKRNYKAM | HLA-B08:01 | SSX1 | Liver; Melanoma; Thyroid | 7406 |
| YMKRNYKAMTKL | HLA-B08:01 | SSX1 | Liver; Melanoma; Thyroid | 7407 |
| KLGFKVTL | HLA-B08:01 | SSX1 | Liver; Melanoma; Thyroid | 7408 |
| EWKKMKYSE | HLA-B08:01 | SSX1 | Liver; Melanoma; Thyroid | 7409 |
| RLRERKQLV | HLA-B08:01 | SSX1 | Liver; Melanoma; Thyroid | 7410 |
| KQLVIYEEI | HLA-B13:02 | SSX1 | Liver; Melanoma; Thyroid | 7411 |
| MTFGRLHRI | HLA-B13:02 | SSX1 | Liver; Melanoma; Thyroid | 7412 |
| KAMTKLGFKV | HLA-B13:02 | SSX1 | Liver; Melanoma; Thyroid | 7413 |
| RLHRIIPKI | HLA-B13:02 | SSX1 | Liver; Melanoma; Thyroid | 7414 |
| RLRERKQLV | HLA-B13:02 | SSX1 | Liver; Melanoma; Thyroid | 7415 |
| KAFDDIATY | HLA-B46:01 | SSX1 | Liver; Melanoma; Thyroid | 7416 |
| YMKRNYKAM | HLA-B46:01 | SSX1 | Liver; Melanoma; Thyroid | 7417 |
| YSEKISYVY | HLA-B46:01 | SSX1 | Liver; Melanoma; Thyroid | 7418 |
| ISYVYMKRNY | HLA-B46:01 | SSX1 | Liver; Melanoma; Thyroid | 7419 |
| YMKRNYKAM | HLA-C01:02 | SSX1 | Liver; Melanoma; Thyroid | 7420 |
| MTFGRLHRI | HLA-C01:02 | SSX1 | Liver; Melanoma; Thyroid | 7421 |
| AFDDIATYF | HLA-C01:02 | SSX1 | Liver; Melanoma; Thyroid | 7422 |
| SGPQNDGKQL | HLA-C01:02 | SSX1 | Liver; Melanoma; Thyroid | 7423 |
| TLPPFMCN | HLA-C01:02 | SSX1 | Liver; Melanoma; Thyroid | 7424 |
| SGPKRGKHAW | HLA-C01:02 | SSX1 | Liver; Melanoma; Thyroid | 7425 |
| MTFGRLHRI | HLA-C03:04 | SSX1 | Liver; Melanoma; Thyroid | 7426 |
| KAFDDIATY | HLA-C03:04 | SSX1 | Liver; Melanoma; Thyroid | 7427 |
| YMKRNYKAM | HLA-C03:04 | SSX1 | Liver; Melanoma; Thyroid | 7428 |
| VTLPPFMCN | HLA-C03:04 | SSX1 | Liver; Melanoma; Thyroid | 7429 |
| FKVTLPPFM | HLA-C03:04 | SSX1 | Liver; Melanoma; Thyroid | 7430 |
| AFDDIATYF | HLA-C04:01 | SSX1 | Liver; Melanoma; Thyroid | 7431 |
| KAFDDIATYF | HLA-C04:01 | SSX1 | Liver; Melanoma; Thyroid | 7432 |
| RSKAFDDIATYF | HLA-C04:01 | SSX1 | Liver; Melanoma; Thyroid | 7433 |
| AFDDIATY | HLA-C04:01 | SSX1 | Liver; Melanoma; Thyroid | 7434 |
| FDDIATYF | HLA-C04:01 | SSX1 | Liver; Melanoma; Thyroid | 7435 |
| MTFGRLHRI | HLA-C07:01 | SSX1 | Liver; Melanoma; Thyroid | 7436 |
| LRERKQLVI | HLA-C07:01 | SSX1 | Liver; Melanoma; Thyroid | 7437 |
| KRNYKAMTKL | HLA-C07:01 | SSX1 | Liver; Melanoma; Thyroid | 7438 |
| FKVTIPPFM | HLA-C07:01 | SSX1 | Liver; Melanoma; Thyroid | 7439 |
| KAFDDIATY | HLA-C07:01 | SSX1 | Liver; Melanoma; Thyroid | 7440 |
| KRGKHAWTH | HLA-C07:01 | SSX1 | Liver; Melanoma; Thyroid | 7441 |
| SYVYMKRNY | HLA-C07:01 | SSX1 | Liver; Melanoma; Thyroid | 7442 |
| MTFGRLHRI | HLA-C07:02 | SSX1 | Liver; Melanoma; Thyroid | 7443 |
| KYSEKISYV | HLA-C07:02 | SSX1 | Liver; Melanoma; Thyroid | 7444 |
| YMKRNYKAM | HLA-C07:02 | SSX1 | Liver; Melanoma; Thyroid | 7445 |
| KYSEKISYVY | HLA-C07:02 | SSX1 | Liver; Melanoma; Thyroid | 7446 |
| KYSEKISY | HLA-C07:02 | SSX1 | Liver; Melanoma; Thyroid | 7447 |
| KRQKHAWTH | HLA-C07:02 | SSX1 | Liver; Melanoma; Thyroid | 7448 |
| YSDQELAY | HLA-A01:01 | STAR | Adrenal Gland | 7449 |
| VVDQPMERLY | HLA-A01:01 | STAR | Adrenal Gland | 7450 |
| YSDQELAYL | HLA-A01:01 | STAR | Adrenal Gland | 7451 |
| AMGEWNPNV | HLA-A02:01 | STAR | Adrenal Gland | 7452 |
| KVMSKVVPDV | HLA-A02:01 | STAR | Adrenal Gland | 7453 |
| VMSKVVPDV | HLA-A02:01 | STAR | Adrenal Gland | 7454 |
| RLYEELVERM | HLA-A02:01 | STAR | Adrenal Gland | 7455 |
| MLLATFKL | HLA-A02:01 | STAR | Adrenal Gland | 7456 |
| AVMAISQEL | HLA-A02:01 | STAR | Adrenal Gland | 7457 |
| KLTWLLSIDI | HLA-A02:01 | STAR | Adrenal Gland | 7458 |
| WLPKSIINQV | HLA-A02:01 | STAR | Adrenal Gland | 7459 |
| RLYEELVERMEA | HLA-A02:01 | STAR | Adrenal Gland | 7460 |
| MLLATFKLCA | HLA-A02:01 | STAR | Adrenal Gland | 7461 |
| RLYEELVER | HLA-A02:01 | STAR | Adrenal Gland | 7462 |
| TLYSDQELA | HLA-A02:01 | STAR | Adrenal Gland | 7463 |
| TLYSDQELAYL | HLA-A02:01 | STAR | Adrenal Gland | 7464 |
| ALGGPTPST | HLA-A02:01 | STAR | Adrenal Gland | 7465 |
| YLQQGEEAM | HLA-A02:01 | STAR | Adrenal Gland | 7466 |
| TLYSDQEL | HLA-A02:01 | STAR | Adrenal Gland | 7467 |
| SSYRHMRNMK | HLA-A03:01 | STAR | Adrenal Gland | 7468 |
| SAWSPTLPLK | HLA-A03:01 | STAR | Adrenal Gland | 7469 |
| KVMSKVVPDVGK | HLA-A03:01 | STAR | Adrenal Gland | 7470 |
| RLYEELVER | HLA-A03:01 | STAR | Adrenal Gland | 7471 |
| AWSPTLPLK | HLA-A03:01 | STAR | Adrenal Gland | 7472 |
| SAWSPTLPLK | HLA-A11:01 | STAR | Adrenal Gland | 7473 |
| SSYRHMRNMK | HLA-A11:01 | STAR | Adrenal Gland | 7474 |
| SIDLKGWLPK | HLA-A11:01 | STAR | Adrenal Gland | 7475 |
| AVMAISQELNR | HLA-A11:01 | STAR | Adrenal Gland | 7476 |
| STWINQVRR | HLA-ALL:01 | STAR | Adrenal Gland | 7477 |
| LYSDQELAYL | HLA-A24:02 | STAR | Adrenal Gland | 7478 |
| RWILPTTCASAW | HLA-A24:02 | STAR | Adrenal Gland | 7479 |
| TWLLSIDLKGW | HLA-A24:02 | STAR | Adrenal Gland | 7480 |
| VVPDVGKVF | HLA-A24:02 | STAR | Adrenal Gland | 7481 |
| VLSQTQVDF | HLA-A24:02 | STAR | Adrenal Gland | 7482 |
| KTKLTWLLS | HLA-A30:01 | STAR | Adrenal Gland | 7483 |
| KTKLTWLLSI | HLA-A30:01 | STAR | Adrenal Gland | 7484 |
| SSYRHMRNMK | HLA-A30:01 | STAR | Adrenal Gland | 7485 |
| NVKEIKVLQK | HLA-A30:01 | STAR | Adrenal Gland | 7486 |
| SYRHMRNMK | HLA-A30:01 | STAR | Adrenal Gland | 7487 |
| CAQLHWYTR | HLA-A33:03 | STAR | Adrenal Gland | 7488 |
| DFVSVRCAKR | HLA-A33:03 | STAR | Adrenal Gland | 7489 |
| FVSVRCAKR | HLA-A33:03 | STAR | Adrenal Gland | 7490 |
| DFANHLRKR | HLA-A33:03 | STAR | Adrenal Gland | 7491 |
| STWINQVRR | HLA-A33:03 | STAR | Adrenal Gland | 7492 |
| RPRWILPTT | HLA-B07:02 | STAR | Adrenal Gland | 7493 |
| RPRWILPTTC | HLA-B07:02 | STAR | Adrenal Gland | 7494 |
| SPSKTKLTWL | HLA-B07:02 | STAR | Adrenal Gland | 7495 |
| LPKSIINQVL | HLA-B07:02 | STAR | Adrenal Gland | 7496 |
| QPAVPNCAQL | HLA-B07:02 | STAR | Adrenal Gland | 7497 |
| CPRPRWIL | HLA-B07:02 | STAR | Adrenal Gland | 7498 |
| NQVRRRSSL | HLA-B08:01 | STAR | Adrenal Gland | 7499 |
| QVRRRSSLL | HLA-B08:01 | STAR | Adrenal Gland | 7500 |
| QVRRRSSL | HLA-B08:01 | STAR | Adrenal Gland | 7501 |
| NVKEIKVL | HLA-B08:01 | STAR | Adrenal Gland | 7502 |
| HLRKRLESH | HLA-B08:01 | STAR | Adrenal Gland | 7503 |
| TLYSDQEL | HLA-B08:01 | STAR | Adrenal Gland | 7504 |
| KEIKVLQKI | HLA-B13:02 | STAR | Adrenal Gland | 7505 |
| MERLYEELV | HLA-B13:02 | STAR | Adrenal Gland | 7506 |
| GEWNPNVKEI | HLA-B13:02 | STAR | Adrenal Gland | 7507 |
| SQQDNGDKY | HLA-B13:02 | STAR | Adrenal Gland | 7508 |
| RLYEELVER | HLA-B13:02 | STAR | Adrenal Gland | 7509 |
| SSYRHMRNM | HLA-B46:01 | STAR | Adrenal Gland | 7510 |
| SAWSPTLPL | HLA-B46:01 | STAR | Adrenal Gland | 7511 |
| YLQQGEEAM | HLA-B46:01 | STAR | Adrenal Gland | 7512 |
| VVPDVGKVF | HLA-B46:01 | STAR | Adrenal Gland | 7513 |
| HLRKRLESH | HLA-B46:01 | STAR | Adrenal Gland | 7514 |
| SAWSPTLPL | HLA-C01:02 | STAR | Adrenal Gland | 7515 |
| YSDQELAYL | HLA-C01:02 | STAR | Adrenal Gland | 7516 |
| CASAWSPTL | HLA-C01:02 | STAR | Adrenal Gland | 7517 |
| ILPTTCASAW | HLA-C01:02 | STAR | Adrenal Gland | 7518 |
| VGPRDFVSV | HLA-C01:02 | STAR | Adrenal Gland | 7519 |
| SAWSPTLPL | HLA-C03:04 | STAR | Adrenal Gland | 7520 |
| LAAEAAGNL | HLA-C03:04 | STAR | Adrenal Gland | 7521 |
| CASAWSPTL | HLA-C03:04 | STAR | Adrenal Gland | 7522 |
| FANHLRKRL | HLA-C03:04 | STAR | Adrenal Gland | 7523 |
| YSDQELAYL | HLA-C04:01 | STAR | Adrenal Gland | 7524 |
| LYEELVERM | HLA-C04:01 | STAR | Adrenal Gland | 7525 |
| SAWSPTLPL | HLA-C04:01 | STAR | Adrenal Gland | 7526 |
| VFRLEVVV | HLA-C04:01 | STAR | Adrenal Gland | 7527 |
| LRQQAVMAI | HLA-C07:01 | STAR | Adrenal Gland | 7528 |
| YRHMRNMKGL | HLA-C07:01 | STAR | Adrenal Gland | 7529 |
| SSYRHMRNM | HLA-C07:01 | STAR | Adrenal Gland | 7530 |
| RRGSTCVL | HLA-C07:01 | STAR | Adrenal Gland | 7531 |
| KRRQSTCVL | HLA-C07:01 | STAR | Adrenal Gland | 7532 |
| RRGSTCVLA | HLA-C07:01 | STAR | Adrenal Gland | 7533 |
| LRQQAVMAI | HLA-C07:02 | STAR | Adrenal Gland | 7534 |
| YRHMRNMIKGL | HLA-C07:02 | STAR | Adrenal Gland | 7535 |
| KRRGSTCVL | HLA-C07:02 | STAR | Adrenal Gland | 7536 |
| IRAEHGPTCM | HLA-C07:02 | STAR | Adrenal Gland | 7537 |
| LYSDQELAY | HLA-C07:02 | STAR | Adrenal Gland | 7538 |
| WSNAISALY | HLA-A01:01 | SYCN | Pancreas | 7539 |
| DWSNAISALY | HLA-A01:01 | SYCN | Pancreas | 7540 |
| HSDGTRICAKLY | HLA-A01:01 | SYCN | Pancreas | 7541 |
| LYDKSDPYY | HLA-A01:01 | SYCN | Pancreas | 7542 |
| LLALALASV | HLA-A02:01 | SYCN | Pancreas | 7543 |
| LLLALALASV | HLA-A02:01 | SYCN | Pancreas | 7544 |
| ILGDWSNAI | HLA-A02:01 | SYCN | Pancreas | 7545 |
| PLLLALALASV | HLA-A02:01 | SYCN | Pancreas | 7546 |
| LLALALASVP | HLA-A02:01 | SYCN | Pancreas | 7547 |
| RPLLLALALASV | HLA-A02:01 | SYCN | Pancreas | 7548 |
| RPLLLALALA | HLA-A02:01 | SYCN | Pancreas | 7549 |
| YLPSNWANTA | HLA-A02:01 | SYCN | Pancreas | 7550 |
| LLLALALASVE | HLA-A02:01 | SYCN | Pancreas | 7551 |
| ALALASVPCA | HLA-A02:01 | SYCN | Pancreas | 7552 |
| KLYDKSDPYY | HLA-A02:01 | SYCN | Pancreas | 7553 |
| PLRPLLLAL | HLA-A02:01 | SYCN | Pancreas | 7554 |
| ALASVPCA | HLA-A02:01 | SYCN | Pancreas | 7555 |
| SLESGADLPYL | HLA-A02:01 | SYCN | Pancreas | 7556 |
| ALASVPCAQ | HLA-A02:01 | SYCN | Pancreas | 7557 |
| KLYDKSDPYY | HLA-A03:01 | SYCN | Pancreas | 7558 |
| KLYDKSDPY | HLA-A03:01 | SYCN | Pancreas | 7559 |
| RTCAKLYDK | HLA-A03:01 | SYCN | Pancreas | 7560 |
| LTVWSRQGK | HLA-A03:01 | SYCN | Pancreas | 7561 |
| GTYPRLEEY | HLA-A03:01 | SYCN | Pancreas | 7562 |
| RTCAKLYDK | HLA-A11:01 | SYCN | Pancreas | 7563 |
| ASSLVVAPR | HLA-A11:01 | SYCN | Pancreas | 7564 |
| GTYPRLEEYR | HLA-A11:01 | SYCN | Pancreas | 7565 |
| GTYPRLEEYRR | HLA-A11:01 | SYCN | Pancreas | 7566 |
| KFSAGTYPRL | HLA-A24:02 | SYCN | Pancreas | 7567 |
| MSPLRPLLL | HLA-A24:02 | SYCN | Pancreas | 7568 |
| YYENCCGGAEL | HLA-A24:02 | SYCN | Pancreas | 7569 |
| EYRRGILGDW | HLA-A24:02 | SYCN | Pancreas | 7570 |
| PYLPSNWANTA | HLA-A24:02 | SYCN | Pancreas | 7571 |
| PYLPSNWAN | HLA-A24:02 | SYCN | Pancreas | 7572 |
| NWANTASSL | HLA-A24:02 | SYCN | Pancreas | 7573 |
| KTHKFSAGT | HLA-A30:01 | SYCN | Pancreas | 7574 |
| KACKTHKFSA | HLA-A30:01 | SYCN | Pancreas | 7575 |
| WSRQGKAGK | HLA-A30:01 | SYCN | Pancreas | 7576 |
| GTRTCAKLY | HLA-A30:01 | SYCN | Pancreas | 7577 |
| RTCAKLYDK | HLA-A30:01 | SYCN | Pancreas | 7578 |
| NAISALYCR | HLA-A33:03 | SYCN | Pancreas | 7579 |
| TASSLVVAPR | HLA-A33:03 | SYCN | Pancreas | 7580 |
| ASSLVVAPR | HLA-A33:03 | SYCN | Pancreas | 7581 |
| TYPRLEEYR | HLA-A33:03 | SYCN | Pancreas | 7582 |
| DLKHSDGTR | HLA-A33:03 | SYCN | Pancreas | 7583 |
| SPLRPLLLAL | HLA-B07:02 | SYCN | Pancreas | 7584 |
| RPLLLALAL | HLA-B07:02 | SYCN | Pancreas | 7585 |
| YPRLEEYRRGIL | HLA-B07:02 | SYCN | Pancreas | 7586 |
| APRCELTVW | HLA-B07:02 | SYCN | Pancreas | 7587 |
| YPRLEEYRRGIL | HLA-B08:01 | SYCN | Pancreas | 7588 |
| SPLRPLLLAL | HLA-B08:01 | SYCN | Pancreas | 7589 |
| MSPLRPLLLAL | HLA-B08:01 | SYCN | Pancreas | 7590 |
| WANTASSI | HLA-B08:01 | SYCN | Pancreas | 7591 |
| SPLRPLLL | HLA-B08:01 | SYCN | Pancreas | 7592 |
| VVAPRCEL | HLA-B08:01 | SYCN | Pancreas | 7593 |
| GDWSNAISA | HLA-B13:02 | SYCN | Pancreas | 7594 |
| GDWSNAISAL | HLA-B13:02 | SYCN | Pancreas | 7595 |
| LESGADLPYL | HLA-B13:02 | SYCN | Pancreas | 7596 |
| RLEEYRRGI | HLA-B13:02 | SYCN | Pancreas | 7597 |
| RQGKAGKT | HLA-B13:02 | SYCN | Pancreas | 7598 |
| RQGKAGKTH | HLA-B13:02 | SYCN | Pancreas | 7599 |
| WSNAISALY | HLA-B46:01 | SYCN | Pancreas | 7600 |
| GTYPRLEEY | HLA-B46:01 | SYCN | Pancreas | 7601 |
| FSAGTYPRL | HLA-B46:01 | SYCN | Pancreas | 7602 |
| KLYDKSDPY | HLA-B46:01 | SYCN | Pancreas | 7603 |
| PLRPLLLAL | HLA-B46:01 | SYCN | Pancreas | 7604 |
| MSPLRPLLL | HLA-C01:02 | SYCN | Pancreas | 7605 |
| FSAGTYPRL | HLA-C01:02 | SYCN | Pancreas | 7606 |
| LVVAPRCEL | HLA-C01:02 | SYCN | Pancreas | 7607 |
| VAPRCELTV | HLA-C01:02 | SYCN | Pancreas | 7608 |
| LRPLLLAL | HLA-C01:02 | SYCN | Pancreas | 7609 |
| FSAGTYPRL | HLA-C03:04 | SYCN | Pancreas | 7610 |
| WANTASSLV | HLA-C03:04 | SYCN | Pancreas | 7611 |
| LSLESGADL | HLA-C03:04 | SYCN | Pancreas | 7612 |
| WANTASSL | HLA-C03:04 | SYCN | Pancreas | 7613 |
| LYDKSDPYY | HLA-C04:01 | SYCN | Pancreas | 7614 |
| YYENCCGGAEL | HLA-C04:01 | SYCN | Pancreas | 7615 |
| MSPLRPLLL | HLA-C04:01 | SYCN | Pancreas | 7616 |
| NWANTASSL | HLA-C04:01 | SYCN | Pancreas | 7617 |
| MSPLRPLLL | HLA-C07:01 | SYCN | Pancreas | 7618 |
| WSNAISALY | HLA-C07:01 | SYCN | Pancreas | 7619 |
| GTYPRLEEY | HLA-C07:01 | SYCN | Pancreas | 7620 |
| YRRGILGD | HLA-C07:01 | SYQN | Pancreas | 7621 |
| ASADLKHSD | HLA-C07:01 | SYCN | Pancreas | 7622 |
| EYRRGILGD | HLA-C07:01 | SYCN | Pancreas | 7623 |
| MSPLRPLLL | HLA-C07:02 | SYCN | Pancreas | 7624 |
| LRPLLLALAL | HLA-C07:02 | SYCN | Pancreas | 7625 |
| WSNAISALY | HLA-C07:02 | SYCN | Pancreas | 7626 |
| LRPLLLAL | HLA-C07:02 | SYCN | Pancreas | 7627 |
| TYPRLEEY | HLA-C07:02 | SYCN | Pancreas | 7628 |
| CVDAEGMEVY | HLA-A01:01 | TG | Thyroid | 7629 |
| FTATSFGHPY | HLA-A01:01 | TG | Thyroid | 7630 |
| FSLFIQSLY | HLA-A01:01 | TG | Thyroid | 7631 |
| RTSSKTAFY | HLA-A01:01 | TG | Thyroid | 7632 |
| YSLEHSTDDY | HLA-A01:01 | TQ | Thyroid | 7633 |
| YSDFSTPLAH | HLA-A01:01 | TG | Thyroid | 7634 |
| STFTETTLY | HLA-A01:01 | TG | Thyroid | 7635 |
| ITESASLYF | HLA-A01:01 | TG | Thyroid | 7636 |
| YSDFSTPLA | HLA-A01:01 | TG | Thyroid | 7637 |
| WVSANIFEY | HLA-A01:01 | TG | Thyroid | 7638 |
| QVDQFLGVPY | HLA-A01:01 | TG | Thyroid | 7639 |
| TTEPEISCDFY | HLA-A01:01 | TG | Thyroid | 7640 |
| FTDLIQSGSF | HLA-A01:01 | TG | Thyroid | 7641 |
| STSPGVSEDCLY | HLA-A01:01 | TG | Thyroid | 7642 |
| STTEPEISCDFY | HLA-A01:01 | TG | Thyroid | 7643 |
| LAAQSTLSFY | HLA-A01:01 | TG | Thyroid | 7644 |
| RVEAAATWYY | HLA-A01:01 | TG | Thyroid | 7645 |
| ITESASLY | HLA-A01:01 | TG | Thyroid | 7646 |
| ILEDKVKNFY | HLA-A01:01 | TG | Thyroid | 7647 |
| SLEHSTDDY | HLA-A01:01 | TG | Thyroid | 7648 |
| LAEITESASLY | HLA-A01:01 | TG | Thyroid | 7649 |
| FTDLIQSGS | HLA-A01:01 | TG | Thyroid | 7650 |
| LLADVQFAL | HLA-A02:01 | TG | Thyroid | 7651 |
| WLFKHLFSA | HLA-A02:01 | TG | Thyroid | 7652 |
| FLLFLQHAI | HLA-A02:01 | TG | Thyroid | 7653 |
| NLFGGKFLV | HLA-A02:01 | TG | Thyroid | 7654 |
| SLLELPEFL | HLA-A02:01 | TG | Thyroid | 7655 |
| TLLASICWV | HLA-A02:01 | TG | Thyroid | 7656 |
| SLACVPCPV | HLA-A02:01 | TG | Thyroid | 7657 |
| YLLCPFPPM | HLA-A02:01 | TG | Thyroid | 7658 |
| FTLLASICWV | HLA-A02:01 | TG | Thyroid | 7659 |
| SLTEKVFKV | HLA-A02:01 | TG | Thyroid | 7660 |
| IMQYFSHFI | HLA-A02:01 | TG | Thyroid | 7661 |
| SLLELPEFLL | HLA-A02:01 | TG | Thyroid | 7662 |
| GLLDQVAAL | HLA-A02:01 | TG | Thyroid | 7663 |
| SLLSYEASV | HLA-A02:01 | TG | Thyroid | 7664 |
| LLDEIYDTI | HLA-A02:01 | TG | Thyroid | 7665 |
| VIFDANAPV | HLA-A02:01 | TG | Thyroid | 7666 |
| YLNVFIPQNY | HLA-A02:01 | TG | Thyroid | 7667 |
| FLAAVGNLI | HLA-A02:01 | TG | Thyroid | 7668 |
| LMMQKFEKV | HLA-A02:01 | TG | Thyroid | 7669 |
| NMLSGLYNPI | HLA-A02:01 | TG | Thyroid | 7670 |
| LLQTFQVFI | HLA-A02:01 | TG | Thyroid | 7671 |
| MLSGLYNPI | HLA-A02:01 | TG | Thyroid | 7672 |
| LLLDEIYDTI | HLA-A02:01 | TG | Thyroid | 7673 |
| ELLADVQFAL | HLA-A02:01 | TG | Thyroid | 7674 |
| FLVNVGQFNL | HLA-A02:01 | TG | Thyroid | 7675 |
| YLLCPFPPMA | HLA-A02:01 | TG | Thyroid | 7676 |
| FLASLLELPEFL | HLA-A02:01 | TG | Thyroid | 7677 |
| FLSSGSGEV | HLA-A02:01 | TG | Thyroid | 7678 |
| GLTTELFSPV | HLA-A02:01 | TG | Thyroid | 7679 |
| KQVDQFLGV | HLA-A02:01 | TG | Thyroid | 7680 |
| SLWVEVDLLI | HLA-A02:01 | TG | Thyroid | 7681 |
| WGLLDQVAAL | HLA-A02:01 | TG | Thyroid | 7682 |
| MLPGLTTEL | HLA-A02:01 | TG | Thyroid | 7683 |
| ALVLEIFTLL | HLA-A02:01 | TG | Thyroid | 7684 |
| QNLFGGKFLV | HLA-A02:01 | TG | Thyroid | 7685 |
| SLLSYEASVPSV | HLA-A02:01 | TQ | Thyroid | 7686 |
| FLAAVGNLIV | HLA-A02:01 | TG | Thyroid | 7687 |
| SLFIQSLYEA | HLA-A02:01 | TG | Thyroid | 7688 |
| HWLFKHLFSA | HLA-A02:01 | TG | Thyroid | 7689 |
| ASLLELPEFL | HLA-A02:01 | TG | Thyroid | 7690 |
| HLMQKFEKV | HLA-A02:01 | TG | Thyroid | 7691 |
| FLASLLEL | HLA-A02:01 | TG | Thyroid | 7692 |
| KIMQYFSHFI | HLA-A02:01 | TG | Thyroid | 7693 |
| LLADVQFALG | HLA-A02:01 | TG | Thyroid | 7694 |
| MLSGLYNPIV | HLA-A02:01 | TG | Thyroid | 7695 |
| ALVLEIFTL | HLA-A02:01 | TG | Thyroid | 7696 |
| GLYNPIVPSA | HLA-A02:01 | TG | Thyroid | 7697 |
| GLREDLLSL | HLA-A02:01 | TG | Thyroid | 7698 |
| SLQDVPLAA | HLA-A02:01 | TG | Thyroid | 7699 |
| SLQDVPLAAL | HLA-A02:01 | TG | Thyroid | 7700 |
| VLNDAQTKL | HLA-A02:01 | TG | Thyroid | 7701 |
| FLVAKGIRL | HLA-A02:01 | TG | Thyroid | 7702 |
| SLYEAGQQDV | HLA-A02:01 | TG | Thyroid | 7703 |
| DLGDVMETV | HLA-A02:01 | TG | Thyroid | 7704 |
| FLREPPARA | HLA-A02:01 | TG | Thyroid | 7705 |
| VIFDANAPVAV | HLA-A02:01 | TG | Thyroid | 7706 |
| GLLSSPSVL | HLA-A02:01 | TG | Thyroid | 7707 |
| SLEEKSLSL | HLA-A02:01 | TG | Thyroid | 7708 |
| FLAAVGNLIVV | HLA-A02:01 | TG | Thyroid | 7709 |
| ELAETGLEL | HLA-A02:01 | TG | Thyroid | 7710 |
| GLLSSPSVLL | HLA-A02:01 | TG | Thyroid | 7711 |
| VLPSLTEKV | HLA-A02:01 | TG | Thyroid | 7712 |
| SLALSSVVV | HLA-A02:01 | TG | Thyroid | 7713 |
| SLWVEVDLL | HLA-A02:01 | TG | Thyroid | 7714 |
| ELPEFLLFL | HLA-A02:01 | TG | Thyroid | 7715 |
| QLAEITESA | HLA-A02:01 | TG | Thyroid | 7716 |
| GLLSSPSV | HLA-A02:01 | TG | Thyroid | 7717 |
| RLEDIPVASI | HLA-A02:01 | TG | Thyroid | 7718 |
| ELAETGLELL | HLA-A02:01 | TG | Thyroid | 7719 |
| GLINRAKAV | HLA-A02:01 | TG | Thyroid | 7720 |
| ALLSNSSML | HLA-A02:01 | TG | Thyroid | 7721 |
| QLLDQVAA | HLA-A02:01 | TG | Thyroid | 7722 |
| TLYPEAQV | HLA-A02:01 | TG | Thyroid | 7723 |
| KLMGISIRINK | HLA-A03:01 | TG | Thyroid | 7724 |
| RLYFGTSGY | HLA-A03:01 | TG | Thyroid | 7725 |
| HTYPFGWYQK | HLA-A03:01 | TG | Thyroid | 7726 |
| RLILPQMPK | HLA-A03:01 | TG | Thyroid | 7727 |
| RTSGLLSSWK | HLA-A03:01 | TG | Thyroid | 7728 |
| CLSFCQLQK | HLA-A03:01 | TG | Thyroid | 7729 |
| SLYFTCTLY | HLA-A03:01 | TG | Thyroid | 7730 |
| CRLILPQMPK | HLA-A03:01 | TG | Thyroid | 7731 |
| RLLHGVGDK | HLA-A03:01 | TG | Thyroid | 7732 |
| ACLSFCQLQK | HLA-A03:01 | TG | Thyroid | 7733 |
| SRLYFGTSGY | HLA-A03:01 | TG | Thyroid | 7734 |
| RLGVNVTWK | HLA-A03:01 | TG | Thyroid | 7735 |
| IIDMASAWAK | HLA-A03:01 | TG | Thyroid | 7736 |
| SLSSQKHWLFK | HLA-A03:01 | TG | Thyroid | 7737 |
| RLQQNLEGGK | HLA-A03:01 | TG | Thyroid | 7738 |
| AIQVGTSWK | HLA-A03:01 | TG | Thyroid | 7739 |
| VVLPSLTEK | HLA-A03:01 | TG | Thyroid | 7740 |
| SVFPPGPLIC | HLA-A03:01 | TG | Thyroid | 7741 |
| SVFPPGPLICS | HLA-A03:01 | TG | Thyroid | 7742 |
| AIFPSRGLAR | HLA-A03:01 | TG | Thyroid | 7743 |
| ATSCPPTIK | HLA-A03:01 | TG | Thyroid | 7744 |
| GLYNPIVFS | HLA-A03:01 | TG | Thyroid | 7745 |
| KLLVKIMSY | HLA-A03:01 | TG | Thyroid | 7746 |
| ILLEPYLFW | HLA-A03:01 | TG | Thyroid | 7747 |
| HTYPFGWYQK | HLA-A11:01 | TG | Thyroid | 7748 |
| STFTETTLYR | HLA-A11:01 | TG | Thyroid | 7749 |
| SSQKHWLFK | HLA-A11:01 | TG | Thyroid | 7750 |
| VVLPSLTEK | HLA-A11:01 | TG | Thyroid | 7751 |
| STLSFYQRR | HLA-A11:01 | TG | Thyroid | 7752 |
| RLILPQMPK | HLA-A11:01 | TG | Thyroid | 7753 |
| IIDMASAWAK | HLA-A11:01 | TG | Thyroid | 7754 |
| GSFQLHLDSK | HLA-A11:01 | TG | Thyroid | 7755 |
| RTSGLLSSWK | HLA-A11:01 | TG | Thyroid | 7756 |
| ATSCPPTIK | HLA-A11:01 | TG | Thyroid | 7757 |
| ATSNFSAVR | HLA-A11:01 | TG | Thyroid | 7758 |
| KLMGISIRNK | HLA-A11:01 | TG | Thyroid | 7759 |
| AFSQTHLMQK | HLA-A11:01 | TG | Thyroid | 7760 |
| VHTYPFGWYQK | HLA-A11:01 | TG | Thyroid | 7761 |
| ATNSQLFRR | HLA-A11:01 | TG | Thyroid | 7762 |
| SSQDDGLINR | HLA-A11:01 | TG | Thyroid | 7763 |
| AVQSVISGR | HLA-A11:01 | TG | Thyroid | 7764 |
| ATPWPDFVPR | HLA-A11:01 | TG | Thyroid | 7765 |
| TTLYRILQR | HLA-A11:01 | TG | Thyroid | 7766 |
| SQDDGLINR | HLA-A11:01 | TG | Thyroid | 7767 |
| SVQVGCLTR | HLA-ALI:01 | TG | Thyroid | 7768 |
| VTFSSPQRR | HLA-A11:01 | TG | Thyroid | 7769 |
| GTFNFSQFFQQ | HLA-A11:01 | TG | Thyroid | 7770 |
| RFPDAFVTF | HLA-A24:02 | TG | Thyroid | 7771 |
| ADYADLLQTF | HLA-A24:02 | TG | Thyroid | 7772 |
| SGYFSQHDLF | HLA-A24:02 | TG | Thyroid | 7773 |
| SYNRFPDAFVTF | HLA-A24:02 | TG | Thyroid | 7774 |
| GYFSQHDLF | HLA-A24:02 | TG | Thyroid | 7775 |
| SYRVGVFGF | HLA-A24:02 | TG | Thyroid | 7776 |
| RWASPRVARF | HLA-A24:02 | TG | Thyroid | 7777 |
| NRFPDAFVTF | HLA-A24:02 | TG | Thyroid | 7778 |
| DYADLLQTF | HLA-A24:02 | TG | Thyroid | 7779 |
| VYLKKGAII | HLA-A24:02 | TG | Thyroid | 7780 |
| RFPDAFVTFSSF | HLA-A24:02 | TG | Thyroid | 7781 |
| SYNREPDAF | HLA-A24:02 | TG | Thyroid | 7782 |
| FYPAYEGQP | HLA-A24:02 | TG | Thyroid | 7783 |
| IFPAETIRF | HLA-A24:02 | TG | Thyroid | 7784 |
| SADYADLLQTF | HLA-A24:02 | TG | Thyroid | 7785 |
| KFLVNVGQF | HLA-A24:02 | TG | Thyroid | 7786 |
| IYDTIFAGL | HLA-A24:02 | TG | Thyroid | 7787 |
| PYMPQCDAF | HLA-A24:02 | TG | Thyroid | 7788 |
| LYFGTSGYF | HLA-A24:02 | TG | Thyroid | 7789 |
| EFMPVQCKF | HLA-A24:02 | TG | Thyroid | 7790 |
| NYKEFSELL | HLA-A24:02 | TG | Thyroid | 7791 |
| EFSRKVPTF | HLA-A24:02 | TG | Thyroid | 7792 |
| QYPGSYSDF | HLA-A24:02 | TG | Thyroid | 7793 |
| FYTRLPPQKL | HLA-A24:02 | TG | Thyroid | 7794 |
| SSQKHWLFK | HLA-A30:01 | TG | Thyroid | 7795 |
| KSRTSGLLS | HLA-A30:01 | TG | Thyroid | 7796 |
| GTRQLGRPK | HLA-A30:01 | TG | Thyroid | 7797 |
| KVKNFYTRL | HLA-A30:01 | TG | Thyroid | 7798 |
| SGRFRCPTK | HLA-A30:01 | TG | Thyroid | 7799 |
| SSKTAFYQA | HLA-A30:01 | TG | Thyroid | 7800 |
| RSQENPSPK | HLA-A30:01 | TG | Thyroid | 7801 |
| GTRGTFNFS | HLA-A30:01 | TG | Thyroid | 7802 |
| KSRTSGLLSS | HLA-A30:01 | TG | Thyroid | 7803 |
| RARGNVFMY | HLA-A30:01 | TG | Thyroid | 7804 |
| GTRSAIGKPK | HLA-A30:01 | TG | Thyroid | 7805 |
| RARMQSLMGS | HLA-A30:01 | TG | Thyroid | 7806 |
| KSRLEDIPV | HLA-A30:01 | TG | Thyroid | 7807 |
| KSRLEDIPVA | HLA-A30:01 | TG | Thyroid | 7808 |
| GTRTSTSPG | HLA-A30:01 | TG | Thyroid | 7809 |
| ATRDYFIIC | HLA-A30:01 | TG | Thyroid | 7810 |
| RNKVPMSEK | HLA-A30:01 | TG | Thyroid | 7811 |
| KLMGISIRNK | HLA-A30:01 | TG | Thyroid | 7812 |
| AKKDGTMNK | HLA-A30:01 | TG | Thyroid | 7813 |
| WSKYISSLK | HLA-A30:01 | TG | Thyroid | 7814 |
| YQRWEAQNK | HLA-A30:01 | TG | Thyroid | 7815 |
| SQKDRGSGK | HLA-A30:01 | TG | Thyroid | 7816 |
| HTYPFGWYQK | HLA-A30:01 | TG | Thyroid | 7817 |
| CTGFGFLNV | HLA-A30:01 | TG | Thyroid | 7818 |
| YTRLPFQKL | HLA-A30:01 | TG | Thyroid | 7819 |
| NFQQVYLWK | HLA-A30:01 | TG | Thyroid | 7820 |
| MPKALFRKK | HLA-A30:01 | TG | Thyroid | 7821 |
| KVILEDKVK | HLA-A30:01 | TG | Thyroid | 7822 |
| MQYFSHFIR | HLA-A33:03 | TG | Thyroid | 7823 |
| STHGRLLGR | HLA-A33:03 | TG | Thyroid | 7824 |
| EAFAEQFLR | HLA-A33:03 | TG | Thyroid | 7825 |
| IMQYFSHFIR | HLA-A33:03 | TG | Thyroid | 7826 |
| TTLYRILQR | HLA-A33:03 | TG | Thyroid | 7827 |
| FVTFSSFQR | HLA-A33:03 | TG | Thyroid | 7828 |
| DMASAWAKR | HLA-A33:03 | TG | Thyroid | 7829 |
| STLSFYQRR | HLA-A33:03 | TG | Thyroid | 7830 |
| LLVKIMSYR | HLA-A33:03 | TG | Thyroid | 7831 |
| MIFDLVHSYNR | HLA-A33:03 | TG | Thyroid | 7832 |
| EAIRAIFPSR | HLA-A33:03 | TG | Thyroid | 7833 |
| HTYPFGWYQK | HLA-A33:03 | TG | Thyroid | 7834 |
| MAGCWAGPR | HLA-A33:03 | TG | Thyroid | 7835 |
| MMIFDLVHSYNR | HLA-A33:03 | TG | Thyroid | 7836 |
| EVYGTRQLGR | HLA-A33:03 | TG | Thyroid | 7837 |
| DANAPVAVR | HLA-A33:03 | TG | Thyroid | 7838 |
| EFSELLPNR | HLA-A33:03 | TG | Thyroid | 7839 |
| YAAPPLAER | HLA-A33:03 | TG | Thyroid | 7840 |
| SSQDDGLINR | HLA-A33:03 | TG | Thyroid | 7841 |
| AVQSVISGR | HLA-A33:03 | TG | Thyroid | 7842 |
| FILDELTAR | HLA-A33:03 | TG | Thyroid | 7843 |
| TLYRILQRR | HLA-A33:03 | TG | Thyroid | 7844 |
| STLSFYQR | HLA-A33:03 | TG | Thyroid | 7845 |
| DVASIHLLTAR | HLA-A33:03 | TG | Thyroid | 7846 |
| DKVKNFYTR | HLA-A33:03 | TG | Thyroid | 7847 |
| EVRQTQLKR | HLA-A33:03 | TG | Thyroid | 7848 |
| RPQPRENIL | HLA-B07:02 | TG | Thyroid | 7849 |
| RPLPFLTPF | HLA-B07:02 | TG | Thyroid | 7850 |
| QPRACQRPQL | HLA-B07:02 | TG | Thyroid | 7851 |
| RPASPTEAGL | HLA-B07:02 | TG | Thyroid | 7852 |
| APENYGHGSL | HLA-B07:02 | TG | Thyroid | 7853 |
| FPSRGLARLAL | HLA-B07:02 | TG | Thyroid | 7854 |
| SPRVARFAT | HLA-B07:02 | TG | Thyroid | 7855 |
| GPRPSRASC | HLA-B07:02 | TG | Thyroid | 7856 |
| RPQPRENILL | HLA-B07:02 | TG | Thyroid | 7857 |
| RPLPFLTPFSSL | HLA-B07:02 | TG | Thyroid | 7858 |
| FPSRGLARL | HLA-B07:02 | TG | Thyroid | 7859 |
| APVAVRSKV | HLA-B07:02 | TG | Thyroid | 7860 |
| VPISTHGRL | HLA-B07:02 | TG | Thyroid | 7861 |
| LPFLTPFSSL | HLA-B07:02 | TG | Thyroid | 7862 |
| YPAYEGQFSL | HLA-B07:02 | TG | Thyroid | 7863 |
| KPMSLDSWQSL | HLA-B07:02 | TG | Thyroid | 7864 |
| APNASVLVF | HLA-B07:02 | TG | Thyroid | 7865 |
| SPDDSAGASAL | HLA-B07:02 | TG | Thyroid | 7866 |
| APSFCPLVVL | HLA-B07:02 | TG | Thyroid | 7867 |
| IYRKPGISL | HLA-B07:02 | TG | Thyroid | 7868 |
| WPAIDGSFL | HLA-B07:02 | TG | Thyroid | 7869 |
| YPSLQDVPL | HLA-B07:02 | TG | Thyroid | 7870 |
| IPVASLPDL | HLA-B07:02 | TG | Thyroid | 7871 |
| SPTEAGLTTEL | HLA-B07:02 | TG | Thyroid | 7872 |
| ILQRRFLAV | HLA-B08:01 | TG | Thyroid | 7873 |
| SAMRHLYLL | HLA-B08:01 | TG | Thyroid | 7874 |
| FLRTVQAL | HLA-B08:01 | TG | Thyroid | 7875 |
| LMMQKFEKV | HLA-B08:01 | TG | Thyroid | 7876 |
| RILQRRFLAV | HLA-B08:01 | TG | Thyroid | 7877 |
| YRILQRRFLAV | HLA-B08:01 | TG | Thyroid | 7878 |
| LQRRFLAV | HLA-B08:01 | TG | Thyroid | 7879 |
| SAMRHLYL | HLA-B08:01 | TG | Thyroid | 7880 |
| KQRARMQSL | HLA-B08:01 | TG | Thyroid | 7881 |
| YQRRRFSPDDSA | HLA-B08:01 | TG | Thyroid | 7882 |
| SQLFRRAVL | HLA-B08:01 | TG | Thyroid | 7883 |
| CLRQKPANVL | HLA-B08:01 | TG | Thyroid | 7884 |
| MPKALFRKKVIL | HLA-B08:01 | TG | Thyroid | 7885 |
| HLMQKFEKV | HLA-B08:01 | TG | Thyroid | 7886 |
| QLFRRAVL | HLA-B08:01 | TG | Thyroid | 7887 |
| CLRQKPANV | HLA-B08:01 | TG | Thyroid | 7888 |
| ALFRKKVIL | HLA-B08:01 | TG | Thyroid | 7889 |
| EAKLRVLQF | HLA-B08:01 | TG | Thyroid | 7890 |
| ECQWREHSL | HLA-B08:01 | TG | Thyroid | 7891 |
| DLLGRFTDL | HLA-B08:01 | TG | Thyroid | 7892 |
| GWYQKPMSL | HLA-B08:01 | TG | Thyroid | 7893 |
| ALKFLASL | HLA-B08:01 | TG | Thyroid | 7894 |
| DAQTKLLAV | HLA-B08:01 | TG | Thyroid | 7895 |
| SLEEKSLSL | HLA-B08:01 | TG | Thyroid | 7896 |
| DPSIRHFDV | HLA-B08:01 | TG | Thyroid | 7897 |
| EACLITTL | HLA-B08:01 | TG | Thyroid | 7898 |
| YRKPGISL | HLA-B08:01 | TG | Thyroid | 7899 |
| CASERQQAL | HLA-B08:01 | TG | Thyroid | 7900 |
| GLINRAKAV | HLA-B08:01 | TG | Thyroid | 7901 |
| DLTPAKLL | HLA-B08:01 | TG | Thyroid | 7902 |
| TDMMIFDLV | HLA-B13:02 | TG | Thyroid | 7903 |
| RDYFIICPI | HLA-B13:02 | TG | Thyroid | 7904 |
| RDYFIICPII | HLA-B13:02 | TG | Thyroid | 7905 |
| LEIFTILLASI | HLA-B13:02 | TG | Thyroid | 7906 |
| CDAFGSWEPV | HLA-B13:02 | TG | Thyroid | 7907 |
| SEQAFLRTV | HLA-B13:02 | TG | Thyroid | 7908 |
| TTDMMIFDLV | HLA-B13:02 | TG | Thyroid | 7909 |
| LEPYLFWQI | HLA-B13:02 | TG | Thyroid | 7910 |
| YEASVPSVPI | HLA-B13:02 | TG | Thyroid | 7911 |
| ADLLQTFQV | HLA-B13:02 | TG | Thyroid | 7912 |
| CTEDEACSFFTV | HLA-B13:02 | TG | Thyroid | 7913 |
| FDLRNCWCV | HLA-B13:02 | TG | Thyroid | 7914 |
| LELPEFLLFL | HLA-B13:02 | TG | Thyroid | 7915 |
| VQFALGLPF | HLA-B13:02 | TG | Thyroid | 7916 |
| RDLCCDGFV | HLA-B13:02 | TG | Thyroid | 7917 |
| DEACSFFTV | HLA-B13:02 | TG | Thyroid | 7918 |
| SQYPSLQDY | HLA-B13:02 | TG | Thyroid | 7919 |
| ALTWVQTHI | HLA-B13:02 | TG | Thyroid | 7920 |
| AQCPSLCNV | HLA-B13:02 | TG | Thyroid | 7921 |
| GQYRASQKD | HLA-B13:02 | TG | Thyroid | 7922 |
| GQWRQVQCN | HLA-B13:02 | TG | Thyroid | 7923 |
| NLFGGKFLV | HLA-B13:02 | TG | Thyroid | 7924 |
| SQTHLMQKF | HLA-B13:02 | TG | Thyroid | 7925 |
| RQSRAPQAL | HLA-B13:02 | TG | Thyroid | 7926 |
| KQVDQFLGV | HLA-B13:02 | TG | Thyroid | 7927 |
| RLLGRSQAI | HLA-B13:02 | TG | Thyroid | 7928 |
| EQTPERLFV | HLA-B13:02 | TG | Thyroid | 7929 |
| GQQDVFPVL | HLA-B13:02 | TG | Thyroid | 7930 |
| VQCDVQQV | HLA-B13:02 | TG | Thyroid | 7931 |
| GQFSLEEKS | HLA-B13:02 | TG | Thyroid | 7932 |
| LAAQSTLSF | HLA-B46:01 | TG | Thyroid | 7933 |
| FMYHAPENY | HLA-B46:01 | TG | Thyroid | 7934 |
| WTSDNVACM | HLA-B46:01 | TG | Thyroid | 7935 |
| YINSTDTSY | HLA-B46:01 | TG | Thyroid | 7936 |
| FSLFIQSLY | HLA-B46:01 | TG | Thyroid | 7937 |
| FTATSFGHPY | HLA-B46:01 | TG | Thyroid | 7938 |
| MIFDLVHSY | HLA-B46:01 | TG | Thyroid | 7939 |
| LAKEVSCPM | HLA-B46:01 | TG | Thyroid | 7940 |
| LAVSGPFHY | HLA-B46:01 | TG | Thyroid | 7941 |
| FQNMLSGLY | HLA-B46:01 | TG | Thyroid | 7942 |
| AASGNFSLF | HLA-B46:01 | TG | Thyroid | 7943 |
| FAGLDLPSTF | HLA-B46:01 | TG | Thyroid | 7944 |
| VQFALGLPF | HLA-B46:01 | TG | Thyroid | 7945 |
| YLLCPFPPM | HLA-B46:01 | TG | Thyroid | 7946 |
| LAAQSTLSFY | HLA-B46:01 | TG | Thyroid | 7947 |
| FGFLNVSQL | HLA-B46:01 | TG | Thyroid | 7948 |
| HSTDDYASF | HLA-B46:01 | TG | Thyroid | 7949 |
| RSKVPDSEF | HLA-B46:01 | TG | Thyroid | 7950 |
| SSRFPLGESF | HLA-B46:01 | TG | Thyroid | 7951 |
| NLIVVTASY | HLA-B46:01 | TG | Thyroid | 7952 |
| PLFPPREAF | HLA-B46:01 | TG | Thyroid | 7953 |
| AARAPGACF | HLA-B46:01 | TG | Thyroid | 7954 |
| VSLDSWQSL | HLA-B46:01 | TG | Thyroid | 7955 |
| RLYFGTSGY | HLA-B46:01 | TG | Thyroid | 7956 |
| SRFPLGESF | HLA-B46:01 | TG | Thyroid | 7957 |
| NATCPGVTY | HLA-B46:01 | TG | Thyroid | 7958 |
| KLLVKIMSY | HLA-B46:01 | TG | Thyroid | 7959 |
| QAIPGTRSA | HLA-B46:01 | TG | Thyroid | 7960 |
| ILRLGDQEF | HLA-B46:01 | TG | Thyroid | 7961 |
| MLPGLTTEL | HLA-C01:02 | TG | Thyroid | 7962 |
| FTNFQQVYL | HLA-C01:02 | TG | Thyroid | 7963 |
| ILPQMPKAL | HLA-C01:02 | TG | Thyroid | 7964 |
| SAMRHLYLL | HLA-C01:02 | TG | Thyroid | 7965 |
| LLSSPSVLL | HLA-C01:02 | TG | Thyroid | 7966 |
| LLADVQFAL | HLA-C01:02 | TG | Thyroid | 7967 |
| ASLYFTCTL | HLA-C01:02 | TG | Thyroid | 7968 |
| SLEEKSLSL | HLA-C01:02 | TG | Thyroid | 7969 |
| YLLCPFPPM | HLA-C01:02 | TG | Thyroid | 7970 |
| CSAMRHLYL | HLA-C01:02 | TG | Thyroid | 7971 |
| RAQQQAIAL | HLA-C01:02 | TG | Thyroid | 7972 |
| FATSCPPTI | HLA-C01:02 | TG | Thyroid | 7973 |
| FGFLNVSQL | HLA-C01:02 | TG | Thyroid | 7974 |
| FSAVRDLCL | HLA-C01:02 | TG | Thyroid | 7975 |
| AAATWYYSL | HLA-C01:02 | TG | Thyroid | 7976 |
| SCPPTIKEL | HLA-C01:02 | TG | Thyroid | 7977 |
| VAPNASVL | HLA-C01:02 | TG | Thyroid | 7978 |
| AIPGTRSAI | HLA-C01:02 | TG | Thyroid | 7979 |
| NAPSFCPL | HLA-C01:02 | TG | Thyroid | 7980 |
| NAPVAVRSK | HLA-C01:02 | TG | Thyroid | 7981 |
| SGPTGSAM | HLA-C01:02 | TG | Thyroid | 7982 |
| VDPASGEEL | HLA-C01:02 | TG | Thyroid | 7983 |
| AQPLRPCEL | HLA-C01:02 | TG | Thyroid | 7984 |
| ELPEFLLFL | HLA-C01:02 | TG | Thyroid | 7985 |
| VSPGYVPAC | HLA-C01:02 | TG | Thyroid | 7986 |
| FEPTGFQNM | HLA-C01:02 | TG | Thyroid | 7987 |
| FSPDDSAGASAL | HLA-C01:02 | TG | Thyroid | 7988 |
| FIPGSLTAR | HLA-C01:02 | TG | Thyroid | 7989 |
| FATSCPPTI | HLA-C03:04 | TG | Thyroid | 7990 |
| WSVFPPGPL | HLA-C03:04 | TG | Thyroid | 7991 |
| LAAQSTLSF | HLA-C03:04 | TG | Thyroid | 7992 |
| MSLDSWQSL | HLA-C03:04 | TG | Thyroid | 7993 |
| FGFLNVSQL | HLA-C03:04 | TG | Thyroid | 7994 |
| AATSNFSAV | HLA-C03:04 | TG | Thyroid | 7995 |
| SALSPAAVI | HLA-C03:04 | TG | Thyroid | 7996 |
| YLLCPFPPM | HLA-C03:04 | TG | Thyroid | 7997 |
| YPAYEGQFSL | HLA-C03:04 | TG | Thyroid | 7998 |
| LAKEVSCPM | HLA-C03:04 | TG | Thyroid | 7999 |
| AAATWYYSL | HLA-C03:04 | TG | Thyroid | 8000 |
| SAMRHLYLL | HLA-C03:04 | TG | Thyroid | 8001 |
| ISGPTGSAM | HLA-C03:04 | TG | Thyroid | 8002 |
| WTSDNVACM | HLA-C03:04 | TG | Thyroid | 8003 |
| AAVGNLIVV | HLA-C03:04 | TG | Thyroid | 8004 |
| CADSQGREL | HLA-C03:04 | TG | Thyroid | 8005 |
| YAAPPLAER | HLA-C03:04 | TG | Thyroid | 8006 |
| FIICPIIDM | HLA-C03:04 | TG | Thyroid | 8007 |
| FATPWPDFV | HLA-C03:04 | TG | Thyroid | 8008 |
| CASERQQAL | HLA-C03:04 | TG | Thyroid | 8009 |
| YASFSRAL | HLA-C03:04 | TG | Thyroid | 8010 |
| RAIFPSRGL | HLA-C03:04 | TG | Thyroid | 8011 |
| PAYEGQFSL | HLA-C03:04 | TG | Thyroid | 8012 |
| LAETGLEL | HLA-C03:04 | TG | Thyroid | 8013 |
| FSLFIQSL | HLA-C03:04 | TG | Thyroid | 8014 |
| FLTPFSSL | HLA-C03:04 | TG | Thyroid | 8015 |
| YADTQSCTHSL | HLA-C03:04 | TG | Thyroid | 8016 |
| FSAVRDLCL | HLA-C03:04 | TG | Thyroid | 8017 |
| FTNFQQVYL | HLA-C03:04 | TG | Thyroid | 8018 |
| IYDTIFAGL | HLA-C04:01 | TG | Thyroid | 8019 |
| RFPDAFVTF | HLA-C04:01 | TG | Thyroid | 8020 |
| SYSDESTPL | HLA-C04:01 | TG | Thyroid | 8021 |
| FYQALQNSL | HLA-C04:01 | TG | Thyroid | 8022 |
| FYQEQAGSI | HLA-C04:01 | TG | Thyroid | 8023 |
| TFPAETIRF | HLA-C04:01 | TG | Thyroid | 8024 |
| RFPLGESFL | HLA-C04:01 | TG | Thyroid | 8025 |
| SQPAGSTLF | HLA-C04:01 | TG | Thyroid | 8026 |
| GQDSPAVYL | HLA-C04:01 | TG | Thyroid | 8027 |
| YREAASGNF | HLA-C04:01 | TG | Thyroid | 8028 |
| LQDVPLAAL | HLA-C04:01 | TG | Thyroid | 8029 |
| FYPAYEGQF | HLA-C04:01 | TG | Thyroid | 8030 |
| SYNRFPDAF | HLA-C04:01 | TG | Thyroid | 8031 |
| MLPGLTTEL | HLA-C04:01 | TG | Thyroid | 8032 |
| TEDEACSFF | HLA-C04:01 | TG | Thyroid | 8033 |
| IFDANAPVAV | HLA-C04:01 | TG | Thyroid | 8034 |
| HFDVAHVST | HLA-C04:01 | TG | Thyroid | 8035 |
| VVDPSIRHF | HLA-C04:01 | TG | Thyroid | 8036 |
| IFDLVHSYN | HLA-C04:01 | TG | Thyroid | 8037 |
| IFDLVHSY | HLA-C04:01 | TG | Thyroid | 8038 |
| CVDEAGQEL | HLA-C04:01 | TG | Thyroid | 8039 |
| HFDVAHVSTA | HLA-C04:01 | TG | Thyroid | 8040 |
| SFCPLVVL | HLA-C04:01 | TG | Thyroid | 8041 |
| TYDQESHQV | HLA-C04:01 | TG | Thyroid | 8042 |
| GQDLTPAKL | HLA-C04:01 | TG | Thyroid | 8043 |
| HFDLRNCWC | HLA-C04:01 | TG | Thyroid | 8044 |
| YRKPGISLI | HLA-C07:01 | TG | Thyroid | 8045 |
| YRILQRRFL | HLA-C07:01 | TG | Thyroid | 8046 |
| RRFLAVQSV | HLA-C07:01 | TG | Thyroid | 8047 |
| TRLPFQKLM | HLA-C07:01 | TG | Thyroid | 8048 |
| YRVGVFGFL | HLA-C07:01 | TG | Thyroid | 8049 |
| SRFPLGESF | HLA-C07:01 | TG | Thyroid | 8050 |
| LREPPARAL | HLA-C07:01 | TG | Thyroid | 8051 |
| WREHSLRPL | HLA-C07:01 | TG | Thyroid | 8052 |
| RRFQAPEPL | HLA-C07:01 | TG | Thyroid | 8053 |
| CRQGSWSVF | HLA-C07:01 | TG | Thyroid | 8054 |
| LRQKPANVL | HLA-C07:01 | TG | Thyroid | 8055 |
| YTRLPFQKLM | HLA-C07:01 | TG | Thyroid | 8056 |
| YREAASGNF | HLA-C07:01 | TG | Thyroid | 8057 |
| ARATNSQLF | HLA-C07:01 | TG | Thyroid | 8058 |
| ISRFPLGESFL | HLA-C07:01 | TG | Thyroid | 8059 |
| QRARMQSLM | HLA-C07:01 | TG | Thyroid | 8060 |
| RRFPEVSGY | HLA-C07:01 | TG | Thyroid | 8061 |
| ARGNVFMY | HLA-C07:01 | TG | Thyroid | 8062 |
| RNPNYPYEF | HLA-C07:01 | TG | Thyroid | 8063 |
| CNGPPEQVF | HLA-C07:01 | TG | Thyroid | 8064 |
| SCPPTIKEL | HLA-C07:01 | TG | Thyroid | 8065 |
| GRNPNYPYEF | HLA-C07:01 | TG | Thyroid | 8066 |
| RARGNVFMY | HLA-C07:01 | TG | Thyroid | 8067 |
| RNGDYQAVQ | HLA-C07:01 | TG | Thyroid | 8068 |
| KRSLWVEVD | HLA-C07:01 | TG | Thyroid | 8069 |
| RRFPEVSG | HLA-C07:01 | TG | Thyroid | 8070 |
| TRDYFIICP | HLA-C07:01 | TG | Thyroid | 8071 |
| MIFDLVHSY | HLA-C07:01 | TG | Thyroid | 8072 |
| RRVSPGYVP | HLA-C07:01 | TG | Thyroid | 8073 |
| YRKPGISLL | HLA-C07:02 | TG | Thyroid | 8074 |
| YRILQRRFL | HLA-C07:02 | TG | Thyroid | 8075 |
| SYSDESTPL | HLA-C07:02 | TG | Thyroid | 8076 |
| FYQALQNSL | HLA-C07:02 | TG | Thyroid | 8077 |
| FYQEQAGSL | HLA-C07:02 | TG | Thyroid | 8078 |
| YRVGVFGFL | HLA-C07:02 | TG | Thyroid | 8079 |
| TRLPFQKLM | HLA-C07:02 | TG | Thyroid | 8080 |
| ARATNSQLF | HLA-C07:02 | TG | Thyroid | 8081 |
| LYFGTSGYF | HLA-C07:02 | TG | Thyroid | 8082 |
| RRFLAVQSV | HLA-C07:02 | TG | Thyroid | 8083 |
| SRFPLGESF | HLA-C07:02 | TG | Thyroid | 8084 |
| CRQGSWSVF | HLA-C07:02 | TG | Thyroid | 8085 |
| FWSKYISSL | HLA-C07:02 | TG | Thyroid | 8086 |
| RRFQAPEPL | HLA-C07:02 | TG | Thyroid | 8087 |
| SYNRFPDAF | HLA-C07:02 | TG | Thyroid | 8088 |
| FYPAYEGQF | HLA-C07:02 | TG | Thyroid | 8089 |
| GRNPNYPYEF | HLA-C07:02 | TG | Thyroid | 8090 |
| YRKPGISL | HLA-C07:02 | TG | Thyroid | 8091 |
| NRFPDAFVTF | HLA-C07:02 | TG | Thyroid | 8092 |
| ARGNVFMY | HLA-C07:02 | TG | Thyroid | 8093 |
| RRFPEVSGY | HLA-C07:02 | TG | Thyroid | 8094 |
| RNPNYPYEF | HLA-C07:02 | TG | Thyroid | 8095 |
| ARVEAAATWY | HLA-C07:02 | TG | Thyroid | 8096 |
| NYKEFSELL | HLA-C07:02 | TG | Thyroid | 8097 |
| NYGHGSLEL | HLA-C07:02 | TG | Thyroid | 8098 |
| NRFPDAFVT | HLA-C07:02 | TG | Thyroid | 8099 |
| ASEVFTSFQY | HLA-A01:01 | TGM4 | Prostate | 8100 |
| WTGDYEGGTAPY | HLA-A01:01 | TGM4 | Prostate | 8101 |
| MTHDSVWNFH | HLA-A01:01 | TGM4 | Prostate | 8102 |
| WTGSAPILQQYY | HLA-A01:01 | TGM4 | Prostate | 8103 |
| GSAPILQQYY | HLA-A01:01 | TGM4 | Prostate | 8104 |
| WTGSAPILQQY | HLA-A01:01 | TGM4 | Prostate | 8105 |
| MASEVFTSFQY | HLA-A01:01 | TGM4 | Prostate | 8106 |
| SSPNAILGKY | HLA-A01:01 | TGM4 | Prostate | 8107 |
| GSAPILQQY | HLA-A01:01 | TGM4 | Prostate | 8108 |
| LNDTGCHY | HLA-A01:01 | TGM4 | Prostate | 8109 |
| ILGSFELQLY | HLA-A01:01 | TGM4 | Prostate | 8110 |
| VLDPRTPSDHY | HLA-A01:01 | TGM4 | Prostate | 8111 |
| DTERNLTVDTY | HLA-A01:01 | TGM4 | Prostate | 8112 |
| VTSSPNAILGKY | HLA-A01:01 | TGM4 | Prostate | 8113 |
| KSEENILY | HLA-A01:01 | TGM4 | Prostate | 8114 |
| LTDVKFSLE | HLA-A01:01 | TGM4 | Prostate | 8115 |
| VMDHAFLLL | HLA-A01:01 | TGM4 | Prostate | 8116 |
| KSEENILYL | HLA-A01:01 | TGM4 | Prostate | 8117 |
| FSEVNGDRL | HLA-A01:01 | TGM4 | Prostate | 8118 |
| LTDVKFSL | HLA-A01:01 | TGM4 | Prostate | 8119 |
| ILNDTGCHYV | HLA-A02:01 | TGM4 | Prostate | 8120 |
| FQYPEFSIEL | HLA-A02:01 | TGM4 | Prostate | 8121 |
| ILGKYQLNV | HLA-A02:01 | TGM4 | Prostate | 8122 |
| LLGNSVNFTV | HLA-A02:01 | TGM4 | Prostate | 8123 |
| QVMDHAFLL | HLA-A02:01 | TGM4 | Prostate | 8124 |
| VMDHAFLLL | HLA-A02:01 | TGM4 | Prostate | 8125 |
| VLDCCISLL | HLA-A02:01 | TGM4 | Prostate | 8126 |
| ILGSFELQL | HLA-A02:01 | TGM4 | Prostate | 8127 |
| RQVMDHAFLL | HLA-A02:01 | TGM4 | Prostate | 8128 |
| WVFAGILTTV | HLA-A02:01 | TGM4 | Prostate | 8129 |
| FIVYDTRFV | HLA-A02:01 | TGM4 | Prostate | 8130 |
| MMSFEKGQGV | HLA-A02:01 | TGM4 | Prostate | 8131 |
| RLIWLVKMV | HLA-A02:01 | TGM4 | Prostate | 8132 |
| IPLTDVKFSL | HLA-A02:01 | TGM4 | Prostate | 8133 |
| VLLGNSVNFTV | HLA-A02:01 | TGM4 | Prostate | 8134 |
| YILNDIGCHYV | HLA-A02:01 | TGM4 | Prostate | 8135 |
| MMDASKELQV | HLA-A02:01 | TGM4 | Prostate | 8136 |
| KMVNGQEEL | HLA-A02:01 | TGM4 | Prostate | 8137 |
| AILGKYQLNV | HLA-A02:01 | TGM4 | Prostate | 8138 |
| AMMSFEKGQGV | HLA-A02:01 | TGM4 | Prostate | 8139 |
| TLAIPLIDV | HLA-A02:01 | TGM4 | Prostate | 8140 |
| IMASEVFTS | HLA-A02:01 | TGM4 | Prostate | 8141 |
| SLAILDDEPV | HLA-A02:01 | TGM4 | Prostate | 8142 |
| SMTHDSVWNFHV | HLA-A02:01 | TGM4 | Prostate | 8143 |
| GQCWVFAGI | HLA-A02:01 | TGM4 | Prostate | 8144 |
| QVMDHAFLLL | HLA-A02:01 | TGM4 | Prostate | 8145 |
| ILTTVLRAL | HLA-A02:01 | TGM4 | Prostate | 8146 |
| FGQCWVFAGI | HLA-A02:01 | TGM4 | Prostate | 8147 |
| DVLLGNSVNFTV | HLA-A02:01 | TGM4 | Prostate | 8148 |
| WVFAGILTT | HLA-A02:01 | TGM4 | Prostate | 8149 |
| EYILNDTGCHYV | HLA-A02:01 | TGM4 | Prostate | 8150 |
| VMDHAFLL | HLA-A02:01 | TGM4 | Prostate | 8151 |
| CIFKNTLAI | HLA-A02:01 | TGM4 | Prostate | 8152 |
| ILDDEPVIR | HLA-A02:01 | TGM4 | Prostate | 8153 |
| ILDDEPVIRG | HLA-A02:01 | TGM4 | Prostate | 8154 |
| PLTDVKFSL | HLA-A02:01 | TGM4 | Prostate | 8155 |
| AILDDEPVI | HLA-A02:01 | TGM4 | Prostate | 8156 |
| TLQNESGKEVTV | HLA-A02:01 | TGM4 | Prostate | 8157 |
| ILDDEPVI | HLA-A02:01 | TGM4 | Prostate | 8158 |
| TLQNESGKEV | HLA-A02:01 | TGM4 | Prostate | 8159 |
| VFAGILTTV | HLA-A02:01 | TGM4 | Prostate | 8160 |
| SLESLGISSL | HLA-A02:01 | TGM4 | Prostate | 8161 |
| VLLGNSVNF | HLA-A02:01 | TGM4 | Prostate | 8162 |
| ALGIPARSV | HLA-A02:01 | TGM4 | Prostate | 8163 |
| VLDPRTPSD | HLA-A02:01 | TGM4 | Prostate | 8164 |
| SIAKHTLVV | HLA-A02:01 | TGM4 | Prostate | 8165 |
| KGYDGWQAV | HLA-A02:01 | TGM4 | Prostate | 8166 |
| FLNQDNAV | HLA-A02:01 | TGM4 | Prostate | 8167 |
| SVQSDDVLL | HLA-A02:01 | TGM4 | Prostate | 8168 |
| SLQTSDHGTV | HLA-A02:01 | TGM4 | Prostate | 8169 |
| ALQNVNILG | HLA-A02:01 | TGM4 | Prostate | 8170 |
| QVSEVTLTL | HLA-A02:01 | TGM4 | Prostate | 8171 |
| YVNENGEKI | HLA-A02:01 | TGM4 | Prostate | 8172 |
| AMCAMMSFEK | HLA-A03:01 | TGM4 | Prostate | 8173 |
| KMAKLCDLNK | HLA-A03:01 | TGM4 | Prostate | 8174 |
| QLYTGKKMAK | HLA-A03:01 | TGM4 | Prostate | 8175 |
| SVNFTVILK | HLA-A03:01 | TGM4 | Prostate | 8176 |
| SLLTESSLK | HLA-A03:01 | TGM4 | Prostate | 8177 |
| ILGKYQLNVK | HLA-A03:01 | TGM4 | Prostate | 8178 |
| ISLLTESSLK | HLA-A03:01 | TGM4 | Prostate | 8179 |
| ISMETTSIGK | HLA-A03:01 | TGM4 | Prostate | 8180 |
| AWMKRPDLPK | HLA-A03:01 | TGM4 | Prostate | 8181 |
| TLAIPLTDVK | HLA-A03:01 | TGM4 | Prostate | 8182 |
| RAMCAMMSFEK | HLA-A03:01 | TGM4 | Prostate | 8183 |
| NSVNFTVILK | HLA-A03:01 | TGM4 | Prostate | 8184 |
| STGPNPSIAK | HLA-A03:01 | TGM4 | Prostate | 8185 |
| AILGKYQLNVK | HLA-A03:01 | TGM4 | Prostate | 8186 |
| KTGPKKFIVK | HLA-A03:01 | TGM4 | Prostate | 8187 |
| VTSSPNAILGK | HLA-A03:01 | TGM4 | Prostate | 8188 |
| YLLFNPWCK | HLA-A03:01 | TGM4 | Prostate | 8189 |
| ILYLLENPWCK | HLA-A03:01 | TGM4 | Prostate | 8190 |
| IIAEIVESK | HLA-A03:01 | TGM4 | Prostate | 8191 |
| VLNQPLQSY | HLA-A03:01 | TGM4 | Prostate | 8192 |
| SVNFTVILK | HLA-A11:01 | TGM4 | Prostate | 8193 |
| AMCAMMSFEK | HLA-A11:01 | TGM4 | Prostate | 8194 |
| ISMETTSIGK | HLA-A11:01 | TGM4 | Prostate | 8195 |
| NSVNFTVILK | HLA-A11:01 | TGM4 | Prostate | 8196 |
| STGPNPSIAK | HLA-ALL:01 | TGM4 | Prostate | 8197 |
| SLLTESSLK | HLA-A11:01 | TGM4 | Prostate | 8198 |
| TSSPNAILGK | HLA-A11:01 | TGM4 | Prostate | 8199 |
| HVWTDAWMK | HLA-A11:01 | TGM4 | Prostate | 8200 |
| GSFELQLYTGKK | HLA-A11:01 | TGM4 | Prostate | 8201 |
| GSFELQLYTGK | HLA-A11:01 | TGM4 | Prostate | 8202 |
| SSPNAILGK | HLA-A11:01 | TGM4 | Prostate | 8203 |
| ATLQNESGK | HLA-A11:01 | TGM4 | Prostate | 8204 |
| SVNFTVILKR | HLA-A11:01 | TGM4 | Prostate | 8205 |
| VTSSPNAILGK | HLA-A11:01 | TGM4 | Prostate | 8206 |
| KTGPKKFIVK | HLA-A11:01 | TGM4 | Prostate | 8207 |
| VNFTVILKR | HLA-A11:01 | TGM4 | Prostate | 8208 |
| QTSSPVFRR | HLA-A11:01 | TGM4 | Prostate | 8209 |
| SYHQLKLEF | HLA-A24:02 | TGM4 | Prostate | 8210 |
| TYINSLAIL | HLA-A24:02 | TGM4 | Prostate | 8211 |
| VFTSFQYPEF | HLA-A24:02 | TGM4 | Prostate | 8212 |
| IFIVYDTRF | HLA-A24:02 | TGM4 | Prostate | 8213 |
| YYNTKQAVCF | HLA-A24:02 | TGM4 | Prostate | 8214 |
| IMASEVFTSF | HLA-A24:02 | TGM4 | Prostate | 8215 |
| RAMCAMMSF | HLA-A24:02 | TGM4 | Prostate | 8216 |
| QYPEFSIEL | HLA-A24:02 | TGM4 | Prostate | 8217 |
| KTYINSLAIL | HLA-A24:02 | TGM4 | Prostate | 8218 |
| VYDTRFVF | HLA-A24:02 | TGM4 | Prostate | 8219 |
| QSYHQLKLEF | HLA-A24:02 | TGM4 | Prostate | 8220 |
| EFQTSSPVF | HLA-A24:02 | TGM4 | Prostate | 8221 |
| VLLGNSVNF | HLA-A24:02 | TGM4 | Prostate | 8222 |
| IFIVYDTRFVF | HLA-A24:02 | TGM4 | Prostate | 8223 |
| IVYDTRFVF | HLA-A24:02 | TGM4 | Prostate | 8224 |
| KTYINSLAI | HLA-A30:01 | TGM4 | Prostate | 8225 |
| AQKIVLITK | HLA-A30:01 | TGM4 | Prostate | 8226 |
| KFIVKLSSK | HLA-A30:01 | TGM4 | Prostate | 8227 |
| MAKLCDLNK | HLA-A30:01 | TGM4 | Prostate | 8228 |
| RAMCAMMSFEK | HLA-A30:01 | TGM4 | Prostate | 8229 |
| KQVKEINAQK | HLA-A30:01 | TGM4 | Prostate | 8230 |
| AIRKGDIFI | HLA-A30:01 | TGM4 | Prostate | 8231 |
| KTGPKKFIVK | HLA-A30:01 | TGM4 | Prostate | 8232 |
| VLRALGIPA | HLA-A30:01 | TGM4 | Prostate | 8233 |
| RPVKENFLHM | HLA-A30:01 | TGM4 | Prostate | 8234 |
| SVNFTVILK | HLA-A30:01 | TGM4 | Prostate | 8235 |
| KMAKLCDLNK | HLA-A30:01 | TGM4 | Prostate | 8236 |
| QVKEINAQK | HLA-A30:01 | TGM4 | Prostate | 8237 |
| RRRDITYEYK | HLA-A30:01 | TGM4 | Prostate | 8238 |
| RRRDITYEY | HLA-A30:01 | TGM4 | Prostate | 8239 |
| RLIWLVKMV | HLA-A30:01 | TGM4 | Prostate | 8240 |
| RKGDIFIVY | HLA-A30:01 | TGM4 | Prostate | 8241 |
| DTRFVFSEV | HLA-A30:01 | TGM4 | Prostate | 8242 |
| NVKTGNHILK | HLA-A30:01 | TGM4 | Prostate | 8243 |
| GDRLIWLVK | HLA-A30:01 | TGM4 | Prostate | 8244 |
| HTLVVLDPR | HLA-A33:03 | TGM4 | Prostate | 8245 |
| HVWTDAWMKR | HLA-A33:03 | TGM4 | Prostate | 8246 |
| HAFLLLSSER | HLA-A33:03 | TGM4 | Prostate | 8247 |
| EFQTSSPVFR | HLA-A33:03 | TGM4 | Prostate | 8248 |
| QTSSPVFRR | HLA-A33:03 | TGM4 | Prostate | 8249 |
| DIFIVYDTR | HLA-A33:03 | TGM4 | Prostate | 8250 |
| SVNFTVILKR | HLA-A33:03 | TGM4 | Prostate | 8251 |
| FQTSSPVFRR | HLA-A33:03 | TGM4 | Prostate | 8252 |
| MVFMPDEDER | HLA-A33:03 | TGM4 | Prostate | 8253 |
| NFHVWTDAWMKR | HLA-A33:03 | TGM4 | Prostate | 8254 |
| STKAVGQDR | HLA-A33:03 | TGM4 | Prostate | 8255 |
| AFLLLSSER | HLA-A33:03 | TGM4 | Prostate | 8256 |
| EFQTSSPVFRR | HLA-A33:03 | TGM4 | Prostate | 8257 |
| ESSLKPTDR | HLA-A33:03 | TGM4 | Prostate | 8258 |
| EVFTSFQYP | HLA-A33:03 | TGM4 | Prostate | 8259 |
| SIELPNTGR | HLA-A33:03 | TGM4 | Prostate | 8260 |
| EFSIELPNTGR | HLA-A33:03 | TGM4 | Prostate | 8261 |
| VNFTVILKR | HLA-A33:03 | TGM4 | Prostate | 8262 |
| SPVFRRGQV | HLA-B07:02 | TGM4 | Prostate | 8263 |
| RDPVLVCRAM | HLA-B07:02 | TGM4 | Prostate | 8264 |
| SPVFRRGQVF | HLA-B07:02 | TGM4 | Prostate | 8265 |
| LPNTGRIGQL | HLA-B07:02 | TGM4 | Prostate | 8266 |
| KPTDRRDPV | HLA-B07:02 | TGM4 | Prostate | 8267 |
| KPTDRRDPVI | HLA-B07:02 | TGM4 | Prostate | 8268 |
| APYKWTGSA | HLA-B07:02 | TGM4 | Prostate | 8269 |
| NPSIAKHTL | HLA-B07:02 | TGM4 | Prostate | 8270 |
| RPVKENFLHM | HLA-B07:02 | TGM4 | Prostate | 8271 |
| PARSVTGF | HLA-B07:02 | TGM4 | Prostate | 8272 |
| APYKWTGSAPI | HLA-B07:02 | TGM4 | Prostate | 8273 |
| FPQCHPGQV | HLA-B07:02 | TGM4 | Prostate | 8274 |
| QPLQSYHQL | HLA-B07:02 | TGM4 | Prostate | 8275 |
| GPKKFIVKL | HLA-B07:02 | TGM4 | Prostate | 8276 |
| GPSPLTAI | HLA-B07:02 | TGM4 | Prostate | 8277 |
| YPEFSIEL | HLA-B07:02 | TGM4 | Prostate | 8278 |
| ILKRKTAAL | HLA-B08:01 | TGM4 | Prostate | 8279 |
| FTVILKRKTAAL | HLA-B08:01 | TGM4 | Prostate | 8280 |
| VILKRKTAAL | HLA-B08:01 | TGM4 | Prostate | 8281 |
| LKRKTAAL | HLA-B08:01 | TGM4 | Prostate | 8282 |
| TVILKRKTAAL | HLA-B08:01 | TGM4 | Prostate | 8283 |
| ILKRKTAALQ | HLA-B08:01 | TGM4 | Prostate | 8284 |
| VILKRKTAA | HLA-B08:01 | TGM4 | Prostate | 8285 |
| ILKRKTAA | HLA-B08:01 | TGM4 | Prostate | 8286 |
| ILKRKTAALQNV | HLA-B08:01 | TGM4 | Prostate | 8287 |
| HLRLVLNQPL | HLA-B08:01 | TGM4 | Prostate | 8288 |
| SIKCKPWNF | HLA-B08:01 | TGM4 | Prostate | 8289 |
| EINAQKIVL | HLA-B08:01 | TGM4 | Prostate | 8290 |
| NPSIAKHTL | HLA-B08:01 | TGM4 | Prostate | 8291 |
| NCIFKNTL | HLA-B08:01 | TGM4 | Prostate | 8292 |
| DSKTYINSL | HLA-B08:01 | TGM4 | Prostate | 8293 |
| NAILGKYQL | HLA-B08:01 | TGM4 | Prostate | 8294 |
| DVKFSLESL | HLA-B08:01 | TGM4 | Prostate | 8295 |
| AILGKYQL | HLA-B08:01 | TGM4 | Prostate | 8296 |
| PLTDVKFSL | HLA-B08:01 | TGM4 | Prostate | 8297 |
| GPKKFIVKL | HLA-B08:01 | TGM4 | Prostate | 8298 |
| WEFQTSSPV | HLA-B13:02 | TGM4 | Prostate | 8299 |
| KENFLHMSV | HLA-B13:02 | TGM4 | Prostate | 8300 |
| RQVMDHAFLL | HLA-B13:02 | TGM4 | Prostate | 8301 |
| LEFSTGPNPSI | HLA-B13:02 | TGM4 | Prostate | 8302 |
| YDTRFVFSEV | HLA-B13:02 | TGM4 | Prostate | 8303 |
| WEFQTSSPVF | HLA-B13:02 | TGM4 | Prostate | 8304 |
| IDFLNQDNAV | HLA-B13:02 | TGM4 | Prostate | 8305 |
| RGFIIAEIV | HLA-B13:02 | TGM4 | Prostate | 8306 |
| KTYINSLAI | HLA-B13:02 | TGM4 | Prostate | 8307 |
| GQCWVFAGI | HLA-B13:02 | TGM4 | Prostate | 8308 |
| SEVNGDRLIWLV | HLA-B13:02 | TGM4 | Prostate | 8309 |
| RLIWLVKMV | HLA-B13:02 | TGM4 | Prostate | 8310 |
| GQLLVCNCI | HLA-B13:02 | TGM4 | Prostate | 8311 |
| GQVFHLRLV | HLA-B13:02 | TGM4 | Prostate | 8312 |
| KGYDGWQAV | HLA-B13:02 | TGM4 | Prostate | 8313 |
| KEINAQKI | HLA-B13:02 | TGM4 | Prostate | 8314 |
| GQEELHVI | HLA-B13:02 | TGM4 | Prostate | 8315 |
| FQYPEFSI | HLA-B13:02 | TGM4 | Prostate | 8316 |
| SIAKHTLVV | HLA-B13:02 | TGM4 | Prostate | 8317 |
| LQSYHQLKL | HLA-B13:02 | TGM4 | Prostate | 8318 |
| MASEVFTSF | HLA-B46:01 | TGM4 | Prostate | 8319 |
| RAMCAMMSF | HLA-B46:01 | TGM4 | Prostate | 8320 |
| IVYDTRFVF | HLA-B46:01 | TGM4 | Prostate | 8321 |
| FIVYDTRFVF | HLA-B46:01 | TGM4 | Prostate | 8322 |
| FAGILTTVL | HLA-B46:01 | TGM4 | Prostate | 8323 |
| FTSFQYPEF | HLA-B46:01 | TGM4 | Prostate | 8324 |
| CRAMCAMMSF | HLA-B46:01 | TGM4 | Prostate | 8325 |
| KTYINSLAI | HLA-B46:01 | TGM4 | Prostate | 8326 |
| YVNENGEKI | HLA-B46:01 | TGM4 | Prostate | 8327 |
| GSAPILQQY | HLA-B46:01 | TGM4 | Prostate | 8328 |
| MASEVFTSF | HLA-B46:01 | TGM4 | Prostate | 8329 |
| LAIPLTDVKF | HLA-B46:01 | TGM4 | Prostate | 8330 |
| VLNQPLQSY | HLA-B46:01 | TGM4 | Prostate | 8331 |
| ILNDIGCHY | HLA-B46:01 | TGM4 | Prostate | 8332 |
| RRRDITYEY | HLA-B46:01 | TGM4 | Prostate | 8333 |
| VSHHTWEF | HLA-B46:01 | TGM4 | Prostate | 8334 |
| SIKCKPWNF | HLA-B46:01 | TGM4 | Prostate | 8335 |
| KGYDGWQAV | HLA-B46:01 | TGM4 | Prostate | 8336 |
| AVSHHTWEF | HLA-B46:01 | TGM4 | Prostate | 8337 |
| SAHDTERNL | HLA-B46:01 | TGM4 | Prostate | 8338 |
| NAVSHHTWEP | HLA-B46:01 | TGM4 | Prostate | 8339 |
| VMDHAFLLL | HLA-C01:02 | TGM4 | Prostate | 8340 |
| VLDCCISLL | HLA-C01:02 | TGM4 | Prostate | 8341 |
| KTYINSLAI | HLA-C01:02 | TGM4 | Prostate | 8342 |
| MASEVFTSP | HLA-C01:02 | TGM4 | Prostate | 8343 |
| FAGILTTVL | HLA-C01:02 | TGM4 | Prostate | 8344 |
| RAMCAMMSF | HLA-C01:02 | TGM4 | Prostate | 8345 |
| VTSSPNAIL | HLA-C01:02 | TGM4 | Prostate | 8346 |
| CGPSPLTAI | HLA-C01:02 | TGM4 | Prostate | 8347 |
| IVYDTRFVF | HLA-C01:02 | TGM4 | Prostate | 8348 |
| KSEENILYL | HLA-C01:02 | TGM4 | Prostate | 8349 |
| CIFKNTLAI | HLA-C01:02 | TGM4 | Prostate | 8350 |
| ESTGPNPSI | HLA-C01:02 | TGM4 | Prostate | 8351 |
| QYPEFSIEL | HLA-C01:02 | TGM4 | Prostate | 8352 |
| SAHDTERNL | HLA-C01:02 | TGM4 | Prostate | 8353 |
| ILTTVLRAL | HLA-C01:02 | TGM4 | Prostate | 8354 |
| FCCGPSPL | HLA-C01:02 | TGM4 | Prostate | 8355 |
| RRPVKENFL | HLA-C01:02 | TGM4 | Prostate | 8356 |
| SSPVFRRGQ | HLA-C01:02 | TGM4 | Prostate | 8357 |
| SSPVFRRGQV | HLA-C01:02 | TGM4 | Prostate | 8358 |
| ILKRKTAAL | HLA-C01:02 | TGM4 | Prostate | 8359 |
| VFCCGPSPL | HLA-C01:02 | TGM4 | Prostate | 8360 |
| MASEVFTSF | HLA-C03:04 | TGM4 | Prostate | 8361 |
| FAGILTTVL | HLA-C03:04 | TGM4 | Prostate | 8362 |
| RAMCAMMSF | HLA-C03:04 | TGM4 | Prostate | 8363 |
| FSTGPNPSI | HLA-C03:04 | TGM4 | Prostate | 8364 |
| AALQNVNIL | HLA-C03:04 | TGM4 | Prostate | 8365 |
| MSVQSDDVL | HLA-C03:04 | TGM4 | Prostate | 8366 |
| IVYDTRFVF | HLA-C03:04 | TGM4 | Prostate | 8367 |
| KTYINSLAI | HLA-C03:04 | TGM4 | Prostate | 8368 |
| IAKHTLVVL | HLA-C03:04 | TGM4 | Prostate | 8369 |
| ISLLTESSL | HLA-C03:04 | TGM4 | Prostate | 8370 |
| MSFEKGQGV | HLA-C03:04 | TGM4 | Prostate | 8371 |
| FQYPEFSIEL | HLA-C03:04 | TGM4 | Prostate | 8372 |
| SAHDTERNL | HLA-C03:04 | TGM4 | Prostate | 8373 |
| QVSEVTLTL | HLA-C03:04 | TGM4 | Prostate | 8374 |
| NAILGKYQL | HLA-C03:04 | TGM4 | Prostate | 8375 |
| KSEENILYL | HLA-C03:04 | TGM4 | Prostate | 8376 |
| VSEVTLTL | HLA-C03:04 | TGM4 | Prostate | 8377 |
| VMDHAFLLL | HLA-C04:01 | TGM4 | Prostate | 8378 |
| VLDCCISLL | HLA-C04:01 | TGM4 | Prostate | 8379 |
| QYPEFSIEL | HLA-C04:01 | TGM4 | Prostate | 8380 |
| RAMCAMMSF | HLA-C04:01 | TGM4 | Prostate | 8381 |
| SFEKGQGVL | HLA-C04:01 | TGM4 | Prostate | 8382 |
| FQYPEFSIEL | HLA-C04:01 | TGM4 | Prostate | 8383 |
| WEFQTSSPVF | HLA-C04:01 | TGM4 | Prostate | 8384 |
| MASEVFTSF | HLA-C04:01 | TGM4 | Prostate | 8385 |
| FSEVNGDRL | HLA-C04:01 | TGM4 | Prostate | 8386 |
| FHVWTDAWM | HLA-C04:01 | TGM4 | Prostate | 8387 |
| EFQTSSPVF | HLA-C04:01 | TGM4 | Prostate | 8388 |
| VYDTRFVFS | HLA-C04:01 | TGM4 | Prostate | 8389 |
| VYDTRFVF | HLA-C04:01 | TGM4 | Prostate | 8390 |
| YYNTKQAVCF | HLA-C04:01 | TGM4 | Prostate | 8391 |
| IFIVYDTRF | HLA-C04:01 | TGM4 | Prostate | 8392 |
| VMDHAFLL | HLA-C04:01 | TGM4 | Prostate | 8393 |
| VFCCGPSPL | HLA-C04:01 | TGM4 | Prostate | 8394 |
| VLDCCISL | HLA-C04:01 | TGM4 | Prostate | 8395 |
| HYNWQATL | HLA-C04:01 | TGM4 | Prostate | 8396 |
| FRRGQVFHL | HLA-C07:01 | TGM4 | Prostate | 8397 |
| RRRDITYEY | HLA-C07:01 | TGM4 | Prostate | 8398 |
| MSFEKGQGV | HLA-C07:01 | TGM4 | Prostate | 8399 |
| HYVGAARSI | HLA-C07:01 | TGM4 | Prostate | 8400 |
| HRRPVKENF | HLA-C07:01 | TGM4 | Prostate | 8401 |
| QVMDHAFLL | HLA-C07:01 | TGM4 | Prostate | 8402 |
| DHYNWQATL | HLA-C07:01 | TGM4 | Prostate | 8403 |
| IRKGDIFIV | HLA-C07:01 | TGM4 | Prostate | 8404 |
| CRAMCAMMSF | HLA-C07:01 | TGM4 | Prostate | 8405 |
| FHVWTDAWM | HLA-C07:01 | TGM4 | Prostate | 8406 |
| KTYINSLAI | HLA-C07:01 | TGM4 | Prostate | 8407 |
| FRRGQVFHLRL | HLA-C07:01 | TGM4 | Prostate | 8408 |
| RRGQVFHL | HLA-C07:01 | TGM4 | Prostate | 8409 |
| RRDPVLVC | HLA-C07:01 | TGM4 | Prostate | 8410 |
| RRDITYEY | HLA-C07:01 | TGM4 | Prostate | 8411 |
| RKGDIFIVY | HLA-C07:01 | TGM4 | Prostate | 8412 |
| KSEENILYL | HLA-C07:01 | TGM4 | Prostate | 8413 |
| RRGQVFHLR | HLA-C07:01 | TGM4 | Prostate | 8414 |
| RRGQVFHLRL | HLA-C07:01 | TGM4 | Prostate | 8415 |
| ERKEYILND | HLA-C07:01 | TGM4 | Prostate | 8416 |
| DRRDPVLVC | HLA-C07:01 | TGM4 | Prostate | 8417 |
| FRRGQVFHL | HLA-C07:02 | TGM4 | Prostate | 8418 |
| SYHQLKLEF | HLA-C07:02 | TGM4 | Prostate | 8419 |
| RRRDITYEY | HLA-C07:02 | TGM4 | Prostate | 8420 |
| TYINSLAIL | HLA-C07:02 | TGM4 | Prostate | 8421 |
| QYPEFSIEL | HLA-C07:02 | TGM4 | Prostate | 8422 |
| HYVGAARSI | HLA-C07:02 | TGM4 | Prostate | 8423 |
| RRPVKENFL | HLA-C07:02 | TGM4 | Prostate | 8424 |
| FQYPEFSIEL | HLA-C07:02 | TGM4 | Prostate | 8425 |
| FHVWTDAWM | HLA-C07:02 | TGM4 | Prostate | 8426 |
| DHYNWQATL | HLA-C07:02 | TGM4 | Prostate | 8427 |
| HRRPVKENF | HLA-C07:02 | TGM4 | Prostate | 8428 |
| KRPDLPKGY | HLA-C07:02 | TGM4 | Prostate | 8429 |
| RKGDIFIVY | HLA-C07:02 | TGM4 | Prostate | 8430 |
| IRKGDIFIVY | HLA-C07:02 | TGM4 | Prostate | 8431 |
| FKNTLAIPL | HLA-C07:02 | TGM4 | Prostate | 8432 |
| AKHTLVVL | HLA-C07:02 | TGM4 | Prostate | 8433 |
| RRGQVFHL | HLA-C07:02 | TGM4 | Prostate | 8434 |
| RGDDANRNY | HLA-A01:01 | TNP1 | Testis | 8435 |
| KRGDDANRNY | HLA-A01:01 | TNP1 | Testis | 8436 |
| SRKRGDDANRNY | HLA-A01:01 | TNP | Testis | 8437 |
| GDDANRNY | HLA-A01:01 | TNP | Testis | 8438 |
| MSTSRKLKS | HLA-A01:01 | TNP1 | Testis | 8439 |
| KLKSHGMRR | HLA-A02:01 | TNP1 | Testis | 8440 |
| KSRSPHKGV | HLA-A02:01 | TNP1 | Testis | 8441 |
| KLKSHGMRRS | HLA-A02:01 | TNP1 | Testis | 8442 |
| RKLKSHGMRR | HLA-A02:01 | TNP1 | Testis | 8443 |
| STSRKLKSHGM | HLA-A02:01 | TNP1 | Testis | 8444 |
| SKSRSPHKGV | HLA-A02:01 | TNP1 | Testis | 8445 |
| MSTSRKLKSHGM | HLA-A02:01 | TNP1 | Testis | 8446 |
| RKYRKGNLK | HLA-A02:01 | TNP1 | Testis | 8447 |
| KSHGMRRSK | HLA-A02:01 | TNP1 | Testis | 8448 |
| KSRSPHKGVK | HLA-A02:01 | TNP1 | Testis | 8449 |
| RGDDANRNY | HLA-A02:01 | TNP1 | Testis | 8450 |
| SRSPHKGV | HLA-A02:01 | TNP1 | Testis | 8451 |
| KRGDDANRN | HLA-A02:01 | TNP1 | Testis | 8452 |
| NLKSRKRGD | HLA-A02:01 | TNP1 | Testis | 8453 |
| ANRNYRSHL | HLA-A02:01 | TNP1 | Testis | 8454 |
| KLKSHGMRRSK | HLA-A03:01 | TNP1 | Testis | 8455 |
| KLKSHGMRR | HLA-A03:01 | TNP1 | Testis | 8456 |
| RSKSRSPHK | HLA-A03:01 | TNP1 | Testis | 8457 |
| RKYRKGNLK | HLA-A03:01 | TNP1 | Testis | 8458 |
| RSPHKGVKR | HLA-A03:01 | TNP1 | Testis | 8459 |
| RSKSRSPHK | HLA-A11:01 | TNP1 | Testis | 8460 |
| KSHGMRRSK | HLA-A11:01 | TNP1 | Testis | 8461 |
| KSRSPHKGVK | HLA-A11:01 | TNP1 | Testis | 8462 |
| GSKRKYRKGNLK | HLA-A11:01 | TNP1 | Testis | 8463 |
| RSPHKGVKR | HLA-A11:01 | TNP1 | Testis | 8464 |
| RKYRKGNLK | HLA-A11:01 | TNP1 | Testis | 8465 |
| STSRKLKSH | HLA-A11:01 | TNP1 | Testis | 8466 |
| KYRKGNLKS | HLA-A24:02 | TNP1 | Testis | 8467 |
| KYRKGNLKSR | HLA-A24:02 | TNP1 | Testis | 8468 |
| KYRKGNLK | HLA-A24:02 | TNP1 | Testis | 8469 |
| RSKSRSPHK | HLA-A30:01 | TNP1 | Testis | 8470 |
| KSHGMRRSK | HLA-A30:01 | TNP1 | Testis | 8471 |
| KSRSPHKGV | HLA-A30:01 | TNP1 | Testis | 8472 |
| HGMRRSKSR | HLA-A33:03 | TNP1 | Testis | 8473 |
| KLKSHGMRR | HLA-A33:03 | TNP1 | Testis | 8474 |
| GVKRGGSKR | HLA-A33:03 | TNP1 | Testis | 8475 |
| YRKGNLKSR | HLA-A33:03 | TNP1 | Testis | 8476 |
| KYRKGNLKSR | HLA-A33:03 | TNP1 | Testis | 8477 |
| KSRSPHKGV | HLA-B07:02 | TNP1 | Testis | 8478 |
| SPHKGVKRG | HLA-B07:02 | TNP1 | Testis | 8479 |
| TSRKLKSHGM | HLA-B07:02 | TNP1 | Testis | 8480 |
| ANRNYRSHL | HLA-B07:02 | TNP1 | Testis | 8481 |
| SPHKGVKR | HLA-B07:02 | TNP1 | Testis | 8482 |
| SRKLKSHGM | HLA-B08:01 | TNP1 | Testis | 8483 |
| SKRKYRKGNL | HLA-B08:01 | TNP1 | Testis | 8484 |
| TSRKLKSHGM | HLA-B08:01 | TNP1 | Testis | 8485 |
| NLKSRKRGD | HLA-B08:01 | TNP1 | Testis | 8486 |
| KRKYRKGNL | HLA-B08:01 | TNP1 | Testis | 8487 |
| TSRKLKSH | HLA-B08:01 | TNP1 | Testis | 8488 |
| KSRSPHKGV | HLA-B13:02 | TNP1 | Testis | 8489 |
| SKSRSPHKGV | HLA-B13:02 | TNP1 | Testis | 8490 |
| ANRNYRSHL | HLA-B13:02 | TNP1 | Testis | 8491 |
| RSPHKGVKR | HLA-B13:02 | TNP1 | Testis | 8492 |
| GVKRGGSKR | HLA-B13:02 | TNP1 | Testis | 8493 |
| RKYRKQNLKS | HLA-B13:02 | TNP1 | Testis | 8494 |
| KSHGMRRSK | HLA-B46:01 | TNP1 | Testis | 8495 |
| KSRSPHKGV | HLA-B46:01 | TNP1 | Testis | 8496 |
| RGDDANRNY | HLA-B46:01 | TNP1 | Testis | 8497 |
| TSRKLKSH | HLA-B46:01 | TNP1 | Testis | 8498 |
| ANRNYRSH | HLA-B46:01 | TNP1 | Testis | 8499 |
| DANRNYRSH | HLA-B46:01 | TNP1 | Testis | 8500 |
| KSRSPHKGV | HLA-C01:02 | TNP1 | Testis | 8501 |
| ANRNYRSHL | HLA-C01:02 | TNP1 | Testis | 8502 |
| MSTSRKLKSHGM | HLA-C01:02 | TNP1 | Testis | 8503 |
| RSPHKGVKR | HLA-C01:02 | TNP1 | Testis | 8504 |
| RSPHKGVK | HLA-C01:02 | TNP1 | Testis | 8505 |
| KSHGMRRSK | HLA-C01:02 | TNP1 | Testis | 8506 |
| KSRSPHKGV | HLA-C03:04 | TNP1 | Testis | 8507 |
| MSTSRKLKSHGM | HLA-C03:04 | TNP1 | Testis | 8508 |
| RGDDANRNY | HLA-C03:04 | TNP1 | Testis | 8509 |
| DANRNYRSHL | HLA-C03:04 | TNP1 | Testis | 8510 |
| DANRNYRSH | HLA-C03:04 | TNP1 | Testis | 8511 |
| ANRNYRSHL | HLA-C03:04 | TNP1 | Testis | 8512 |
| KLKSHGMRR | HLA-C04:01 | TNP1 | Testis | 8513 |
| KSRSPHKGV | HLA-C04:01 | TNP1 | Testis | 8514 |
| RGDDANRNY | HLA-C04:01 | TNP1 | Testis | 8515 |
| RGDDANRN | HLA-C04:01 | TNP1 | Testis | 8516 |
| NRNYRSHL | HLA-C04:01 | TNP1 | Testis | 8517 |
| SRKLKSHGM | HLA-C07:01 | TNP1 | Testis | 8518 |
| KRKYRKGNL | HLA-C07:01 | TNP1 | Testis | 8519 |
| YRKGNLKSR | HLA-C07:01 | TNP1 | Testis | 8520 |
| KRGGSKRKY | HLA-C07:01 | TNP1 | Testis | 8521 |
| KRGDDANRNY | HLA-C07:01 | TNP1 | Testis | 8522 |
| RRSKSRSP | HLA-C07:01 | TNP1 | Testis | 8523 |
| SRKLKSHGM | HLA-C07:02 | TNP1 | Testis | 8524 |
| KRKYRKGNL | HLA-C07:02 | TNP1 | Testis | 8525 |
| YRKGNLKSR | HLA-C07:02 | TNP1 | Testis | 8526 |
| KRGGSKRKY | HLA-C07:02 | TNP1 | Testis | 8527 |
| NRNYRSHL | HLA-C07:02 | TNP1 | Testis | 8528 |
| SRSPHKGVK | HLA-C07:02 | TNP1 | Testis | 8529 |
| WLDVQVSNTY | HLA-A01:01 | TPD52L3 | Testis | 8530 |
| RTETSVGTY | HLA-A01:01 | TPD52L3 | Testis | 8531 |
| ARTETSVGTY | HLA-A01:01 | TPD52L3 | Testis | 8532 |
| LTEPEQREL | HLA-A01:01 | TPD52L3 | Testis | 8533 |
| KLGLTALVGL | HLA-A02:01 | TPD52L3 | Testis | 8534 |
| KLEAEIVTL | HLA-A02:01 | TPD52L3 | Testis | 8535 |
| GLMGTIKSKV | HLA-A02:01 | TPD52L3 | Testis | 8536 |
| KLTKLEAEI | HLA-A02:01 | TPD52L3 | Testis | 8537 |
| ALSTMGTLI | HLA-A02:01 | TPD52L3 | Testis | 8538 |
| GLIFNKYTL | HLA-A02:01 | TPD52L3 | Testis | 8539 |
| TLICRKLGGV | HLA-A02:01 | TPD52L3 | Testis | 8540 |
| RSFEGLMGTI | HLA-A02:01 | TPD52L3 | Testis | 8541 |
| KLGLTALV | HLA-A02:01 | TPD52L3 | Testis | 8542 |
| ALVGLRQNL | HLA-A02:01 | TPD52L3 | Testis | 8543 |
| KLGGVKKSA | HLA-A02:01 | TPD52L3 | Testis | 8544 |
| GLTALVGL | HLA-A02:01 | TPD52L3 | Testis | 8545 |
| TKLEAEIVTL | HLA-A02:01 | TPD52L3 | Testis | 8546 |
| RSFEGLIFNK | HLA-A03:01 | TPD52L3 | Testis | 8547 |
| STMGTLICRK | HLA-A03:01 | TPD52L3 | Testis | 8548 |
| VTLRHVLAAK | HLA-A03:01 | TPD52L3 | Testis | 8549 |
| GLMGTIKSK | HLA-A03:01 | TPD52L3 | Testis | 8550 |
| RSFEGLMGTIK | HLA-A03:01 | TPD52L3 | Testis | 8551 |
| STMGTLICRK | HLA-A11:01 | TPD52L3 | Testis | 8552 |
| RSFEGLIFNK | HLA-A11:01 | TPD52L3 | Testis | 8553 |
| STMGTLICR | HLA-A11:01 | TPD52L3 | Testis | 8554 |
| TFRSFEGLIF | HLA-A24:02 | TPD52L3 | Testis | 8555 |
| SFEGLIFNKYTL | HLA-A24:02 | TPD52L3 | Testis | 8556 |
| SFEGLMGTI | HLA-A24:02 | TPD52L3 | Testis | 8557 |
| TYESHSTSEL | HLA-A24:02 | TPD52L3 | Testis | 8558 |
| KKSATFRSF | HLA-A24:02 | TPD52L3 | Testis | 8559 |
| KTKLTKLEA | HLA-A30:01 | TPD52L3 | Testis | 8560 |
| RSFEGLIFNK | HLA-A30:01 | TPD52L3 | Testis | 8561 |
| KTSAALSTM | HLA-A30:01 | TPD52L3 | Testis | 8562 |
| VSNTYVKQK | HLA-A30:01 | TPD52L3 | Testis | 8563 |
| RSFEGLIFN | HLA-A30:01 | TPD52L3 | Testis | 8564 |
| STMGTLICR | HLA-A33:03 | TPD52L3 | Testis | 8565 |
| HVLAAKERR | HLA-A33:03 | TPD52L3 | Testis | 8566 |
| LSTMGTLICR | HLA-A33:03 | TPD52L3 | Testis | 8567 |
| GVKKSATFR | HLA-A33:03 | TPD52L3 | Testis | 8568 |
| EDLTEPEQR | HLA-A33:03 | TPD52L3 | Testis | 8569 |
| EAEIVTLR | HLA-A33:03 | TPD52L3 | Testis | 8570 |
| MPHARTETSV | HLA-B07:02 | TPD52L3 | Testis | 8571 |
| YVKQKTSAAL | HLA-B07:02 | TPD52L3 | Testis | 8572 |
| MPHARTETS | HLA-B07:02 | TPD52L3 | Testis | 8573 |
| NPKGEGSRI | HLA-B07:02 | TPD52L3 | Testis | 8574 |
| SAALSTMGTL | HLA-B07:02 | TPD52L3 | Testis | 8575 |
| YVKQKTSAAL | HLA-B08:01 | TPD52L3 | Testis | 8576 |
| YVKQKTSAA | HLA-B08:01 | TPD52L3 | Testis | 8577 |
| ELKTKLTKL | HLA-B08:01 | TPD52L3 | Testis | 8578 |
| ELKRKLGL | HLA-B08:01 | TPD52L3 | Testis | 8579 |
| LIFNKYTL | HLA-B08:01 | TPD52L3 | Testis | 8580 |
| RSFEGLMGTI | HLA-B13:02 | TPD52L3 | Testis | 8581 |
| AEIVTLRHV | HLA-B13:02 | TPD52L3 | Testis | 8582 |
| LDVQVSNTYV | HLA-B13:02 | TPD52L3 | Testis | 8583 |
| ALSTMGTLI | HLA-B13:02 | TPD52L3 | Testis | 8584 |
| KLEAEIVTL | HLA-B13:02 | TPD52L3 | Testis | 8585 |
| NLSKSWLDV | HLA-B13:02 | TPD52L3 | Testis | 8586 |
| KTSAALSTM | HLA-B46:01 | TPD52L3 | Testis | 8587 |
| YVKQKTSAA | HLA-B46:01 | TPD5213 | Testis | 8588 |
| AALSTMGTL | HLA-B46:01 | TPD52L3 | Testis | 8589 |
| TSVGTYESH | HLA-B46:01 | TPD52L3 | Testis | 8590 |
| RSFEGLIF | HLA-B46:01 | TPD52L3 | Testis | 8591 |
| KTSAALSTM | HLA-C01:02 | TPD52L3 | Testis | 8592 |
| AALSTMGTL | HLA-C01:02 | TPD52L3 | Testis | 8593 |
| YVKQKTSAAL | HLA-C01:02 | TPD52L3 | Testis | 8594 |
| TEPEQREL | HLA-C01:02 | TPD52L3 | Testis | 8595 |
| ESHSTSEL | HLA-C01:02 | TPD52L3 | Testis | 8596 |
| YESHSTSEL | HLA-C01:02 | TPD52L3 | Testis | 8597 |
| AALSTMGTL | HLA-C03:04 | TPD52L3 | Testis | 8598 |
| KTSAALSTM | HLA-C03:04 | TPD52L3 | Testis | 8599 |
| SAALSTMGTL | HLA-C03:04 | TPD52L3 | Testis | 8600 |
| LTEPEQREL | HLA-C03:04 | TPD52L3 | Testis | 8601 |
| YESHSTSEL | HLA-C03:04 | TPD52L3 | Testis | 8602 |
| YESHSTSEL | HLA-C04:01 | TPD52L3 | Testis | 8603 |
| FRSFEGLIF | HLA-C04:01 | TPD52L3 | Testis | 8604 |
| SFEGLMGTI | HLA-C04:01 | TPD52L3 | Testis | 8605 |
| TYESHSTSEL | HLA-C04:01 | TPD52L3 | Testis | 8606 |
| SFEGLIFN | HLA-C04:01 | TPD52L3 | Testis | 8607 |
| KLEAEIVTL | HLA-C04:01 | TPD52L3 | Testis | 8608 |
| FRSFEGLIP | HLA-C07:01 | TPD52L3 | Testis | 8609 |
| KRKLGLTAL | HLA-C07:01 | TPD52L3 | Testis | 8610 |
| FRSFEGLM | HLA-C07:01 | TPD52L3 | Testis | 8611 |
| RSFEGLIF | HLA-C07:01 | TPD52L3 | Testis | 8612 |
| LTEPEQREL | HLA-C07:01 | TPD52L3 | Testis | 8613 |
| KSATFRSF | HLA-C07:01 | TPD52L3 | Testis | 8614 |
| FRSFEGLIF | HLA-C07:02 | TPD52L3 | Testis | 8615 |
| KRKLGLTAL | HLA-C07:02 | TPD52L3 | Testis | 8616 |
| TFRSFEGLM | HLA-C07:02 | TPD52L3 | Testis | 8617 |
| ARTETSVGTY | HLA-C07:02 | TPD52L3 | Testis | 8618 |
| FADFCMGMY | HLA-A01:01 | TSHR | Thyroid | 8619 |
| SVDLYTHSEY | HLA-A01:01 | TSHR | Thyroid | 8620 |
| SVDLYTHSEYY | HLA-A01:01 | TSHR | Thyroid | 8621 |
| DTETPLALAY | HLA-A01:01 | TSHR | Thyroid | 8622 |
| VIDKDAFGGVY | HLA-A01:01 | TSHR | Thyroid | 8623 |
| FLLALLPLV | HLA-A02:01 | TSHR | Thyroid | 8624 |
| FLMCNLAFA | HLA-A02:01 | TSHR | Thyroid | 8625 |
| SLFSWLYRL | HLA-A02:01 | TSHR | Thyroid | 8626 |
| KMFPDLTKV | HLA-A02:01 | TSHR | Thyroid | 8627 |
| ALLGNVFVL | HLA-A02:01 | TSHR | Thyroid | 8628 |
| CFLLALLPLV | HLA-A02:01 | TSHR | Thyroid | 8629 |
| FLYAIFTKA | HLA-A02:01 | TSHR | Thyroid | 8630 |
| SLFSWLYRLPL | HLA-A02:01 | TSHR | Thyroid | 8631 |
| CMAPISFYA | HLA-A02:01 | TSHR | Thyroid | 8632 |
| KSLFSWLYRL | HLA-A02:01 | TSHR | Thyroid | 8633 |
| RFLMCNLAFA | HLA-A02:01 | TSHR | Thyroid | 8634 |
| SLLALLGNV | HLA-A02:01 | TSHR | Thyroid | 8635 |
| SLLDVSQTSV | HLA-A02:01 | TSHR | Thyroid | 8636 |
| ILNKPLITV | HLA-A02:01 | TSHR | Thyroid | 8637 |
| SLLVENVAV | HLA-A02:01 | TSHR | Thyroid | 8638 |
| FLRIVVWFV | HLA-A02:01 | TSHR | Thyroid | 8639 |
| KLYNNGFTSV | HLA-A02:01 | TSHR | Thyroid | 8640 |
| ALLGNVFV | HLA-A02:01 | TSHR | Thyroid | 8641 |
| ALAYIVFVL | HLA-A02:01 | TSHR | Thyroid | 8642 |
| ITFAMRLDRK | HLA-A03:01 | TSHR | Thyroid | 8643 |
| WLYRLPLGRK | HLA-A03:01 | TSHR | Thyroid | 8644 |
| ALKELPLLK | HLA-A03:01 | TSHR | Thyroid | 8645 |
| KLDAVYLNK | HLA-A03:01 | TSHR | Thyroid | 8646 |
| ILLSKFGICK | HLA-A03:01 | TSHR | Thyroid | 8647 |
| SLPPSTQTLK | HLA-A03:01 | TSHR | Thyroid | 8648 |
| ITFAMRLDRK | HLA-A11:01 | TSHR | Thyroid | 8649 |
| LILLTSHYK | HLA-A11:01 | TSHR | Thyroid | 8650 |
| KSLFSWLYR | HLA-A11:01 | TSHR | Thyroid | 8651 |
| AIFTKAFQR | HLA-A11:01 | TSHR | Thyroid | 8652 |
| AVSGKQFCK | HLA-A11:01 | TSHR | Thyroid | 8653 |
| SSMQSLRQR | HLA-A11:01 | TSHR | Thyroid | 8654 |
| KSLSFETQK | HLA-A11:01 | TSHR | Thyroid | 8655 |
| VYSTDIFFI | HLA-A24:02 | TSHR | Thyroid | 8656 |
| HYKLNVPRF | HLA-A24:02 | TSHR | Thyroid | 8657 |
| SYPSHCCAF | HLA-A24:02 | TSHR | Thyroid | 8658 |
| LYAIFTKAF | HLA-A24:02 | TSHR | Thyroid | 8659 |
| AYIVFVLTL | HLA-A24:02 | TSHR | Thyroid | 8660 |
| IYVSIDVTL | HLA-A24:02 | TSHR | Thyroid | 8661 |
| PYMTSIPVNAF | HLA-A24:02 | TSHR | Thyroid | 8662 |
| IARNTWTLK | HLA-A30:01 | TSHR | Thyroid | 8663 |
| KIRLRHACA | HLA-A30:01 | TSHR | Thyroid | 8664 |
| AYRGQRVPPK | HLA-A30:01 | TSHR | Thyroid | 8665 |
| RTIPSHAFS | HLA-A30:01 | TSHR | Thyroid | 8666 |
| KLDAVYLNK | HLA-A30:01 | TSHR | Thyroid | 8667 |
| GYKFLRIVV | HLA-A30:01 | TSHR | Thyroid | 8668 |
| KNKYLTVIDK | HLA-A30:01 | TSHR | Thyroid | 8669 |
| ALKELPLLK | HLA-A30:01 | TSHR | Thyroid | 8670 |
| DIMGYKFLR | HLA-A33:03 | TSHR | Thyroid | 8671 |
| YAIFTKAFQR | HLA-A33:03 | TSHR | Thyroid | 8672 |
| EDIMGYKFLR | HLA-A33:03 | TSHR | Thyroid | 8673 |
| AIFTKAFQR | HLA-A33:03 | TSHR | Thyroid | 8674 |
| WYAITFAMR | HLA-A33:03 | TSHR | Thyroid | 8675 |
| EHLKELIAR | HLA-A33:03 | TSHR | Thyroid | 8676 |
| YVKIYITVR | HLA-A33:03 | TSHR | Thyroid | 8677 |
| LPMDTETPL | HLA-B07:02 | TSHR | Thyroid | 8678 |
| RPADLLQLVL | HLA-B07:02 | TSHR | Thyroid | 8679 |
| IPSHAFSNL | HLA-B07:02 | TSHR | Thyroid | 8680 |
| LPLGRKSLSF | HLA-B07:02 | TSHR | Thyroid | 8681 |
| IPSLPPSTQTL | HLA-B07:02 | TSHR | Thyroid | 8682 |
| APISFYAL | HLA-B07:02 | TSHR | Thyroid | 8683 |
| RPADLLQL | HLA-B07:02 | TSHR | Thyroid | 8684 |
| SLRQRKSVNAL | HLA-B08:01 | TSHR | Thyroid | 8685 |
| YLNKNKYLTV | HLA-B08:01 | TSHR | Thyroid | 8686 |
| TLKKLPLSL | HLA-B08:01 | TSHR | Thyroid | 8687 |
| FSWLYRLPL | HLA-B08:01 | TSHR | Thyroid | 8688 |
| DLLQLVLL | HLA-B08:01 | TSHR | Thyroid | 8689 |
| TWTLKKLPL | HLA-B08:01 | TSHR | Thyroid | 8690 |
| MRLDRKIRL | HLA-B08:01 | TSHR | Thyroid | 8691 |
| TDIFFILEI | HLA-B13:02 | TSHR | Thyroid | 8692 |
| TDFICMAPI | HLA-B13:02 | TSHR | Thyroid | 8693 |
| ADFCMGMYLLLI | HLA-B13:02 | TSHR | Thyroid | 8694 |
| FTDFICMAPI | HLA-B13:02 | TSHR | Thyroid | 8695 |
| AQAYRGQRV | HLA-B13:02 | TSHR | Thyroid | 8696 |
| RPADLLQLV | HLA-B13:02 | TSHR | Thyroid | 8697 |
| KMFPDLTKV | HLA-B13:02 | TSHR | Thyroid | 8698 |
| GQISEEYMQ | HLA-B13:02 | TSHR | Thyroid | 8699 |
| YAFNGTKLDAVY | HLA-B46:01 | TSHR | Thyroid | 8700 |
| FTSVQGYAF | HLA-B46:01 | TSHR | Thyroid | 8701 |
| YAKVSICLPM | HLA-B46:01 | TSHR | Thyroid | 8702 |
| MTSIPVNAF | HLA-B46:01 | TSHR | Thyroid | 8703 |
| LSYPSHCCAF | HLA-B46:01 | TSHR | Thyroid | 8704 |
| YAIFTKAF | HLA-B46:01 | TSHR | Thyroid | 8705 |
| VAFVIVCCCY | HLA-B46:01 | TSHR | Thyroid | 8706 |
| ALNSPLHQEY | HLA-B46:01 | TSHR | Thyroid | 8707 |
| SLPPSTQTL | HLA-C01:02 | TSHR | Thyroid | 8708 |
| MAPISFYAL | HLA-C01:02 | TSHR | Thyroid | 8709 |
| FSWLYRLPL | HLA-C01:02 | TSHR | Thyroid | 8710 |
| YAITFAMRL | HLA-C01:02 | TSHR | Thyroid | 8711 |
| RLPLGRKSL | HLA-C01:02 | TSHR | Thyroid | 8712 |
| LLPLGRKSL | HLA-C01:02 | TSHR | Thyroid | 8713 |
| YAITFAMRL | HLA-C03:04 | TSHR | Thyroid | 8714 |
| ISFYALSAI | HLA-C03:04 | TSHR | Thyroid | 8715 |
| HAFSNLPNI | HLA-C03:04 | TSHR | Thyroid | 8716 |
| FSWLYRLPL | HLA-C03:04 | TSHR | Thyroid | 8717 |
| YSTDIFFIL | HLA-C03:04 | TSHR | Thyroid | 8718 |
| LAYIVEVLTL | HLA-C03:04 | TSHR | Thyroid | 8719 |
| FADFCMGMYL | HLA-C03:04 | TSHR | Thyroid | 8720 |
| VYSGPSLLL | HLA-C04:01 | TSHR | Thyroid | 8721 |
| FADFCMGMYL | HLA-C04:01 | TSHR | Thyroid | 8722 |
| SYPSHCCAF | HLA-C04:01 | TSHR | Thyroid | 8723 |
| SLPPSTQTL | HLA-C04:01 | TSHR | Thyroid | 8724 |
| MFPDLTKVY | HLA-C04:01 | TSHR | Thyroid | 8725 |
| FFEEQEDEI | HLA-C04:01 | TSHR | Thyroid | 8726 |
| IFNTGLKMF | HLA-C04:01 | TSHR | Thyroid | 8727 |
| FYNLSKVTHI | HLA-C04:01 | TSHR | Thyroid | 8728 |
| VFVLLILL | HLA-C04:01 | TSHR | Thyroid | 8729 |
| MRLDRKIRL | HLA-C07:01 | TSHR | Thyroid | 8730 |
| IRNTRNLTY | HLA-C07:01 | TSHR | Thyroid | 8731 |
| LRHACAIMV | HLA-C07:01 | TSHR | Thyroid | 8732 |
| YKLNVPREL | HLA-C07:01 | TSHR | Thyroid | 8733 |
| MRPADLLQL | HLA-C07:01 | TSHR | Thyroid | 8734 |
| HNMEDVYEL | HLA-C07:01 | TSHR | Thyroid | 8735 |
| HNNAHYYVF | HLA-C07:01 | TSHR | Thyroid | 8736 |
| KRMAVLIF | HLA-C07:01 | TSHR | Thyroid | 8737 |
| MRPADLLQL | HLA-C07:02 | TSHR | Thyroid | 8738 |
| IRNTRNLTY | HLA-C07:02 | TSHR | Thyroid | 8739 |
| MRLDRKIRL | HLA-C07:02 | TSHR | Thyroid | 8740 |
| SYPSHCCAF | HLA-C07:02 | TSHR | Thyroid | 8741 |
| AYIVFVLTL | HLA-C07:02 | TSHR | Thyroid | 8742 |
| HNNAHYYVF | HLA-C07:02 | TSHR | Thyroid | 8743 |
| IYVSIDVTL | HLA-C07:02 | TSHR | Thyroid | 8744 |
| YQDWQQNLY | HLA-A01:01 | TSPAN10 | Melanoma | 8745 |
| ASYQDWQQNLY | HLA-A01:01 | TSPAN10 | Melanoma | 8746 |
| VSAVSLAGY | HLA-A01:01 | TSPAN10 | Melanoma | 8747 |
| LLDQVQLGL | HLA-A01:01 | TSPAN10 | Melanoma | 8748 |
| FLSNFPFSL | HLA-A02:01 | TSPAN10 | Melanoma | 8749 |
| FLSNFPFSLL | HLA-A02:01 | TSPAN10 | Melanoma | 8750 |
| FLLDQVQLGL | HLA-A02:01 | TSPANIQ | Melanoma | 8751 |
| IFLSNFPPSL | HLA-A02:01 | TSPAN10 | Melanoma | 8752 |
| FLVLEAVAGA | HLA-A02:01 | TSPAN10 | Melanoma | 8753 |
| ALAIGLWGL | HLA-A02:01 | TSPAN10 | Melanoma | 8754 |
| SLLGLLALA | HLA-A02:01 | TSPAN10 | Melanoma | 8755 |
| YLIFLSNFPFSL | HLA-A02:01 | TSPAN10 | Melanoma | 8756 |
| GLLALAIGL | HLA-A02:01 | TSPAN10 | Melanoma | 8757 |
| FLLDQVQL | HLA-A02:01 | TSPAN10 | Melanoma | 8758 |
| ALGGLVVSA | HLA-A02:01 | TSPAN10 | Melanoma | 8759 |
| GLALGGLVV | HLA-A02:01 | TSPAN10 | Melanoma | 8760 |
| SLAGYLGAL | HLA-A02:01 | TSPAN10 | Melanoma | 8761 |
| AIGLWGLAVK | HLA-A03:01 | TSPAN10 | Melanoma | 8762 |
| SLSPGSSCVK | HLA-A03:01 | TSPAN10 | Melanoma | 8763 |
| VVSAVSLAGY | HLA-A03:01 | TSPAN10 | Melanoma | 8764 |
| ALAIGLWGLAVK | HLA-A03:01 | TSPAN10 | Melanoma | 8765 |
| RLLGALAAR | HLA-A03:01 | TSPAN10 | Melanoma | 8766 |
| SLSPGSSCVK | HLA-A11:01 | TSPAN10 | Melanoma | 8767 |
| AIGLWGLAVK | HLA-A11:01 | TSPAN10 | Melanoma | 8768 |
| VVSAVSLAGY | HLA-A11:01 | TSPAN10 | Melanoma | 8769 |
| AIAVVLLQG | HLA-A11:01 | TSPAN10 | Melanoma | 8770 |
| AVAGALVVA | HLA-A11:01 | TSPAN10 | Melanoma | 8771 |
| KYLIFLSNF | HLA-A24:02 | TSPAN10 | Melanoma | 8772 |
| KYLIFLSNFPF | HLA-A24:02 | TSPAN10 | Melanoma | 8773 |
| VKYLIFLSNF | HLA-A24:02 | TSPAN10 | Melanoma | 8774 |
| SYQDWQQNLYF | HLA-A24:02 | TSPAN10 | Melanoma | 8775 |
| SYQDWQQNL | HLA-A24:02 | TSPAN10 | Melanoma | 8776 |
| AARLLGALA | HLA-A30:01 | TSPAN10 | Melanoma | 8777 |
| AARRGAAYG | HLA-A30:01 | TSPAN10 | Melanoma | 8778 |
| RGFSGGILA | HLA-A30:01 | TSPAN10 | Melanoma | 8779 |
| SYQDWQQNL | HLA-A30:01 | TSPAN10 | Melanoma | 8780 |
| RFLLDQVQL | HLA-A30:01 | TSPAN10 | Melanoma | 8781 |
| DQCGFGVLR | HLA-A33:03 | TSPANI0 | Melanoma | 8782 |
| CGPPLRRWLR | HLA-A33:03 | TSPAN10 | Melanoma | 8783 |
| RLLGALAAR | HLA-A33:03 | TSPAN10 | Melanoma | 8784 |
| DSLEHTLR | HLA-A33:03 | TSPAN10 | Melanoma | 8785 |
| HYQDDPDLR | HLA-A33:03 | TSPAN10 | Melanoma | 8786 |
| TPKKGPAPSL | HLA-B07:02 | TSPAN10 | Melanoma | 8787 |
| LPADPMLGLAL | HLA-B07:02 | TSPAN10 | Melanoma | 8788 |
| SPGVQACSL | HLA-B07:02 | TSPAN10 | Melanoma | 8789 |
| LPADPMLQL | HLA-B07:02 | TSPAN10 | Melanoma | 8790 |
| APSLSPGSSC | HLA-B07:02 | TSPAN10 | Melanoma | 8791 |
| LLAARLLGAL | HLA-B08:01 | TSPAN10 | Melanoma | 8792 |
| ALAARRGAA | HLA-B08:01 | TSPAN10 | Melanoma | 8793 |
| FLSNFPFSL | HLA-B08:01 | TSPAN10 | Melanoma | 8794 |
| DAAQRVVYL | HLA-B08:01 | TSPAN10 | Melanoma | 8795 |
| PPETKHQAL | HLA-B08:01 | TSPAN10 | Melanoma | 8796 |
| DPMLGLAL | HLA-B08:01 | TSPAN10 | Melanoma | 8797 |
| LEAVAGALV | HLA-B13:02 | TSPAN10 | Melanoma | 8798 |
| LEAVAGALVV | HLA-B13:02 | TSPAN10 | Melanoma | 8799 |
| LAFLVLEAV | HLA-B13:02 | TSPAN10 | Melanoma | 8800 |
| GLALGGLVV | HLA-B13:02 | TSPAN10 | Melanoma | 8801 |
| GLWGLAVKG | HLA-B13:02 | TSPAN10 | Melanoma | 8802 |
| RLDADAAQRV | HLA-B13:02 | TSPAN10 | Melanoma | 8803 |
| LAARRGAAY | HLA-B46:01 | TSPAN10 | Melanoma | 8804 |
| FLSNFPFSL | HLA-B46:01 | TSPAN10 | Melanoma | 8805 |
| LAARLLGAL | HLA-B46:01 | TSPAN10 | Melanoma | 8806 |
| FSLLGLLAL | HLA-B46:01 | TSPAN10 | Melanoma | 8807 |
| SSCVKYLIF | HLA-B46:01 | TSPAN10 | Melanoma | 8808 |
| VHRPPTSGC | HLA-B46:01 | TSPAN10 | Melanoma | 8809 |
| TLRVAIAHY | HLA-B46:01 | TSPAN10 | Melanoma | 8810 |
| FLSNFPFSL | HLA-C01:02 | TSPAN10 | Melanoma | 8811 |
| LSNFPFSLL | HLA-C01:02 | TSPAN10 | Melanoma | 8812 |
| YLEGCGPPL | HLA-C01:02 | TSPAN10 | Melanoma | 8813 |
| SSPGVQACSL | HLA-C01:02 | TSPAN10 | Melanoma | 8814 |
| NFPFSLLGL | HLA-C01:02 | TSPAN10 | Melanoma | 8815 |
| ADPMLGLAL | HLA-C01:02 | TSPAN10 | Melanoma | 8816 |
| LAASGGYAI | HLA-C03:04 | TSPAN10 | Melanoma | 8817 |
| FSLLGLLAL | HLA-C03:04 | TSPAN10 | Melanoma | 8818 |
| LAARLLGAL | HLA-C03:04 | TSPAN10 | Melanoma | 8819 |
| YAIAVVLL | HLA-C03:04 | TSPAN10 | Melanoma | 8820 |
| LSNFPFSLL | HLA-C03:04 | TSPAN10 | Melanoma | 8821 |
| YQDDPDLRF | HLA-C04:01 | TSPAN10 | Melanoma | 8822 |
| FLSNFPFSL | HLA-C04:01 | TSPAN10 | Melanoma | 8823 |
| YQDWQQNLY | HLA-C04:01 | TSPAN10 | Melanoma | 8824 |
| NFPFSLLGL | HLA-C04:01 | TSPAN10 | Melanoma | 8825 |
| GYAIAVVLL | HLA-C04:01 | TSPAN10 | Melanoma | 8826 |
| LRRWLRANL | HLA-C07:01 | TSPAN10 | Melanoma | 8827 |
| FLSNFPFSL | HLA-C07:01 | TSPAN10 | Melanoma | 8828 |
| HRPPTSGCL | HLA-C07:01 | TSPAN10 | Melanoma | 8829 |
| RGFSGGILAF | HLA-C07:01 | TSPAN10 | Melanoma | 8830 |
| CLGPVPRED | HLA-C07:01 | TSPAN10 | Melanoma | 8831 |
| SYQDWQQNL | HLA-C07:02 | TSPAN10 | Melanoma | 8832 |
| FLSNFPFSL | HLA-C07:02 | TSPAN10 | Melanoma | 8833 |
| HRPPTSGCL | HLA-C07:02 | TSPAN10 | Melanoma | 8834 |
| KKGPAPSL | HLA-C07:02 | TSPAN10 | Melanoma | 8835 |
| SSCPAFLRY | HLA-A01:01 | UBQLN3 | Testis | 8836 |
| YTDIMDPML | HLA-A01:01 | UBQLN3 | Testis | 8837 |
| RSSCPAFLRY | HLA-A01:01 | UBQLN3 | Testis | 8838 |
| LTGLSRLGLAY | HLA-A01:01 | UBQLN3 | Testis | 8839 |
| YTDIMDPMLN | HLA-A01:01 | UBQLN3 | Testis | 8840 |
| YTDIMDPMLNAV | HLA-A01:01 | UBQLN3 | Testis | 8841 |
| RLYDYLQQL | HLA-A02:01 | UBQLN3 | Testis | 8842 |
| IMDPMLNAV | HLA-A02:01 | UBQLN3 | Testis | 8843 |
| FLSPPFLHML | HLA-A02:01 | UBQLN3 | Testis | 8844 |
| SLMRQHVSV | HLA-A02:01 | UBQLN3 | Testis | 8845 |
| FLHMLQDLV | HLA-A02:01 | UBQLN3 | Testis | 8846 |
| IRLYDYLQQL | HLA-A02:01 | UBQLN3 | Testis | 8847 |
| LLWFMPCLA | HLA-A02:01 | UBQLN3 | Testis | 8848 |
| LLLWFMPCL | HLA-A02:01 | UBQLN3 | Testis | 8849 |
| FLSPPFLHM | HLA-A02:01 | UBQLN3 | Testis | 8850 |
| GLLTGLSRL | HLA-A02:01 | UBQLN3 | Testis | 8851 |
| VQDPHLIKV | HLA-A02:01 | UBQLN3 | Testis | 8852 |
| GLGDSANRV | HLA-A02:01 | UBQLN3 | Testis | 8853 |
| GLLSNTGLV | HLA-A02:01 | UBQLN3 | Testis | 8854 |
| VLATEAPRL | HLA-A02:01 | UBQLN3 | Testis | 8855 |
| QLHENPQSL | HLA-A02:01 | UBQLN3 | Testis | 8856 |
| RLYDYLQQLH | HLA-A03:01 | UBQLN3 | Testis | 8857 |
| LIKVTVKTPK | HLA-A03:01 | UBQLN3 | Testis | 8858 |
| APVQDPHLIK | HLA-A03:01 | UBQLN3 | Testis | 8859 |
| GLSRLGLAYR | HLA-A03:01 | UBQLN3 | Testis | 8860 |
| RLYDYLQQL | HLA-A03:01 | UBQLN3 | Testis | 8861 |
| RLLLWFMPC | HLA-A03:01 | UBQLN3 | Testis | 8862 |
| GLSRLGLAY | HLA-A03:01 | UBQLN3 | Testis | 8863 |
| RSSCPAFLR | HLA-A11:01 | UBQLN3 | Testis | 8864 |
| APVQDPHLIK | HLA-A11:01 | UBQLN3 | Testis | 8865 |
| TDTCTIQQLK | HLA-A11:01 | UBQLN3 | Testis | 8866 |
| SSCPAFLRY | HLA-A11:01 | UBQLN3 | Testis | 8867 |
| SVAGGIESR | HLA-A11:01 | UBQLN3 | Testis | 8868 |
| PVQDPHLIK | HLA-A11:01 | UBQLN3 | Testis | 8869 |
| RFPNFLGII | HLA-A24:02 | UBQLN3 | Testis | 8870 |
| IYPADGPPAF | HLA-A24:02 | UBQLN3 | Testis | 8871 |
| RVPFAPLSF | HLA-A24:02 | UBQLN3 | Testis | 8872 |
| TYLQGTASAL | HLA-A24:02 | UBQLN3 | Testis | 8873 |
| MYTDIMDPML | HLA-A24:02 | UBQLN3 | Testis | 8874 |
| PLPNPPPEVF | HLA-A24:02 | UBQLN3 | Testis | 8875 |
| GFPDQPSSL | HLA-A24:02 | UBQLN3 | Testis | 8876 |
| IRNRFPNFLGI | HLA-A30:01 | UBQLN3 | Testis | 8877 |
| KGRSSCPAFL | HLA-A30:01 | UBQLN3 | Testis | 8878 |
| RSMGFLNREA | HLA-A30:01 | UBQLN3 | Testis | 8879 |
| LSRLGLAYR | HLA-A30:01 | UBQLN3 | Testis | 8880 |
| PVQDPHLIK | HLA-A30:01 | UBQLN3 | Testis | 8881 |
| RLYDYLQQL | HLA-A30:01 | UBQLN3 | Testis | 8882 |
| RQIEQGLQV | HLA-A30:01 | UBQLN3 | Testis | 8883 |
| GVRDGLTVHL | HLA-A30:01 | UBQLN3 | Testis | 8884 |
| LSRLGLAYR | HLA-A33:03 | UBQLN3 | Testis | 8885 |
| QVLATEAPR | HLA-A33:03 | UBQLN3 | Testis | 8886 |
| IMRQTLEFLR | HLA-A33:03 | UBQLN3 | Testis | 8887 |
| MANPRALQALR | HLA-A33:03 | UBQLN3 | Testis | 8888 |
| SVAGGIESR | HLA-A33:03 | UBQLN3 | Testis | 8889 |
| DAPDIRNR | HLA-A33:03 | UBQLN3 | Testis | 8890 |
| QLKEEISQR | HLA-A33:03 | UBQLN3 | Testis | 8891 |
| ESVAIKGR | HLA-A33:03 | UBQLN3 | Testis | 8892 |
| YPRSLRPDGM | HLA-B07:02 | UBQLN3 | Testis | 8893 |
| SPAYPRSL | HLA-B07:02 | UBQLN3 | Testis | 8894 |
| LPQGSPAPV | HLA-B07:02 | UBQLN3 | Testis | 8895 |
| LPSPAYPRSL | HLA-B07:02 | UBQLN3 | Testis | 8896 |
| FPALDSAEL | HLA-B07:02 | UBQLN3 | Testis | 8897 |
| YPADGPPAF | HLA-B07:02 | UBQLN3 | Testis | 8898 |
| LPNPPPEVF | HLA-B07:02 | UBQLN3 | Testis | 8899 |
| NPQSLGTYL | HLA-B07:02 | UBQLN3 | Testis | 8900 |
| SLMRQHVSV | HLA-B08:01 | UBQLN3 | Testis | 8901 |
| VIKRQHRAM | HLA-B08:01 | UBQLN3 | Testis | 8902 |
| LMRQHVSV | HLA-B08:01 | UBQLN3 | Testis | 8903 |
| LVIKRQHRAM | HLA-B08:01 | UBQLN3 | Testis | 8904 |
| DPHLIKVTV | HLA-B08:01 | UBQLN3 | Testis | 8905 |
| ILKDPDSL | HLA-B08:01 | UBQLN3 | Testis | 8906 |
| TGLVRQLVL | HLA-B08:01 | UBQLN3 | Testis | 8907 |
| DQPPAFSL | HLA-B08:01 | UBQLN3 | Testis | 8908 |
| RQIEQGLQV | HLA-B13:02 | UBQLN3 | Testis | 8909 |
| RDGLTVHLVI | HLA-B13:02 | UBQLN3 | Testis | 8910 |
| RDGLTVHLV | HLA-B13:02 | UBQLN3 | Testis | 8911 |
| LSFSPTAAI | HLA-B13:02 | UBQLN3 | Testis | 8912 |
| LESIPGGYNV | HLA-B13:02 | UBQLN3 | Testis | 8913 |
| RLYDYLQQL | HLA-B13:02 | UBQLN3 | Testis | 8914 |
| VQDPHLIKV | HLA-B13:02 | UBQLN3 | Testis | 8915 |
| HLIKVTVKT | HLA-B13:02 | UBQLN3 | Testis | 8916 |
| FSVTDTCTI | HLA-B46:01 | UBQLN3 | Testis | 8917 |
| FLSPPFLHM | HLA-B46:01 | UBQLN3 | Testis | 8918 |
| LSFSPTAAI | HLA-B46:01 | UBQLN3 | Testis | 8919 |
| AAMANPRAL | HLA-B46:01 | UBQLN3 | Testis | 8920 |
| NAIKGRSSC | HLA-B46:01 | UBQLN3 | Testis | 8921 |
| AAIPGIPEP | HLA-B46:01 | UBQLN3 | Testis | 8922 |
| QLHENPQSL | HLA-B46:01 | UBQLN3 | Testis | 8923 |
| YPADGPPAF | HLA-B46:01 | UBQLN3 | Testis | 8924 |
| SVPEFVTQL | HLA-C01:02 | UBQLN3 | Testis | 8925 |
| LSPPFLHML | HLA-C01:02 | UBQLN3 | Testis | 8926 |
| YLQGTASAL | HLA-C01:02 | UBQLN3 | Testis | 8927 |
| FLSPPFLHM | HLA-C01:02 | UBQLN3 | Testis | 8928 |
| SIPGGYNVL | HLA-C01:02 | UBQLN3 | Testis | 8929 |
| NLPDLVSGL | HLA-C01:02 | UBQLN3 | Testis | 8930 |
| PSPAYPRSL | HLA-C01:02 | UBQLN3 | Testis | 8931 |
| AAMANPRAL | HLA-C03:04 | UBQLN3 | Testis | 8932 |
| LSFSPTAAI | HLA-C03:04 | UBQLN3 | Testis | 8933 |
| FSVTDTCTI | HLA-C03:04 | UBQLN3 | Testis | 8934 |
| FSLGLLTGL | HLA-C03:04 | UBQLN3 | Testis | 8935 |
| FLSPPFLHM | HLA-C03:04 | UBQLN3 | Testis | 8936 |
| KAHPDQLVL | HLA-C03:04 | UBQLN3 | Testis | 8937 |
| LATEAPRLL | HLA-C03:04 | UBQLN3 | Testis | 8938 |
| FPDQPSSLM | HLA-C04:01 | UBQLN3 | Testis | 8939 |
| MYTDIMDPM | HLA-C04:01 | UBQLN3 | Testis | 8940 |
| YTDIMDPML | HLA-C04:01 | UBQLN3 | Testis | 8941 |
| FLSPPFLHM | HLA-C04:01 | UBQLN3 | Testis | 8942 |
| LYDYLQQL | HLA-C04:01 | UBQLN3 | Testis | 8943 |
| IMDPMLNAV | HLA-C04:01 | UBQLN3 | Testis | 8944 |
| VRDGLTVHL | HLA-C04:01 | UBQLN3 | Testis | 8945 |
| VFPALDSAEL | HLA-C04:01 | UBQLN3 | Testis | 8946 |
| IRNRFPNFL | HLA-C07:01 | UBQLN3 | Testis | 8947 |
| NRFPNELGI | HLA-C07:01 | UBQLN3 | Testis | 8948 |
| GRSSCPAFL | HLA-C07:01 | UBQLN3 | Testis | 8949 |
| RLYDYLQQL | HLA-C07:01 | UBQLN3 | Testis | 8950 |
| IRLYDYLQQL | HLA-C07:01 | UBQLN3 | Testis | 8951 |
| VRDGLTVHL | HLA-C07:01 | UBQLN3 | Testis | 8952 |
| IRSQDRVL | HLA-C07:01 | UBQLN3 | Testis | 8953 |
| SIPGGYNVL | HLA-C07:01 | UBQLN3 | Testis | 8954 |
| KAHPDQLVL | HLA-C07:01 | UBQLN3 | Testis | 8955 |
| IRNRFPNFL | HLA-C07:02 | UBQLN3 | Testis | 8956 |
| NRFPNFLGI | HLA-C07:02 | UBQLN3 | Testis | 8957 |
| RLYDYLQQL | HLA-C07:02 | UBQLN3 | Testis | 8958 |
| MYTDIMDPM | HLA-C07:02 | UBQLN3 | Testis | 8959 |
| NRVPFAPLSF | HLA-C07:02 | UBQLN3 | Testis | 8960 |
| MRQHVSVPEF | HLA-C07:02 | UBQLN3 | Testis | 8961 |
| NRVPFAPL | HLA-C07:02 | UBQLN3 | Testis | 8962 |

TABLE 1B
Tumor Epitope Sequences

| | SEQ | | | HLA | MS | | Allele | |
|---|---|---|---|---|---|---|---|---|
| Epitope | ID NO: | Target | Sample | class | validation | Allele (MS) | (predicted) | Cancer |
| AYSEKVTEF | 3534 | KLK2 | MDAPCa2b | I | X | HLA-C*06:02 | HLA- | Prostate |
| | | | | | | | A24:02 | |
| GLWTGGKDTCGV | 3468 | KLK2 | MDAPCa2b | I | | | HLA- | Prostate |
| | | | | | | | A02:01 | |
| HPEDTGQVF | 3988 | KLK3 | MDAPCa2b, | I | X | HLA-C*07:01 | HLA- | Prostate |
| | | | human prostate | | | HLA-C*04:01 | C07:01 | |
| HPEYNRPLL | 4143 | KLK4 | MDAPCa2b, | I | X | HLA-C*07:01 | HLA- | Prostate |
| | | | human prostate, | | | HLA-B*07:02 | B07:02 | |
| | | | breast cancer | | | | | |
| QRVPVSHSF | 3544 | KLK2 | MDAPCa2b, | I | X | HLA-C*07:01 | HLA- | Prostate |
| | | | human prostate | | | HLA-C*07:02 | A24:02 | |
| SESDTIRSI | 4176 | KLK4 | mantle cell | I | | | HLA- | Prostate |
| | | | lymphoma, | | | | B13:02 | |
| | | | breast cancer | | | | | |
| SLFHPEDTGQV | 3775 | KLK3 | MDAPCa2b | I | | | HLA- | Prostate |
| | | | | | | | A02:01 | |
| SLQCVSLHL | 3456 | KLK2 | MDAPCa2b, | I | | | HLA- | Prostate |
| | | | human prostate | | | | A02:01 | |
| VILLGRHSL | 3891 | KLK3 | PXD004023 | I | | | HLA- | Prostate |
| | | | | | | | B08:01 | |
| VLVHPQWVL | 3757 | KLK3 | MDAPCa2b | I | | | HLA | Prostate |
| | | | | | | | A02:01 | |
| LFHPEDTGQVF | 3827 | KLK3 | Human prostate | I | X | HLA-A*24:02 | HLA- | Prostate |
| | | | | | | | A24:02 | |
| RPRSLQCVSL | 3578 | KLK2 | Human prostate | I | | | HLA- | Prostate |
| | | | | | | | B07:02 | |
| GYLQGLVSF | 4094 | KLK4 | Human prostate | I | X | HLA-A*24:02 | HLA- | Prostate |
| | | | | | | | A24:02 | |
| IRNKSVILL | 3974 | KLK3 | Human prostate | I | X | HLA-C*06:02 | HLA- | Prostate |
| | | | | | | HLA-C*07:02 | C07:01 | |
| KLQCVDLHV | 3740 | KLK3 | Human prostate | I | X | HLA-A*02:01 | HLA- | Prostate |
| | | | | | | | A02:01 | |
| LLANGRMPTV | 4029 | KLK4 | Human prostate | I | X | HLA-A*02:01 | HLA- | Prostate |
| | | | | | | | A02:01 | |
| LRPGDDSTL | 3767 | KLK3 | Human prostate | I | X | HLA-C*07:02 | HLA- | Prostate |
| | | | | | | | C07:02 | |
| MPALPMVL | 3874 | KLK3 | Human prostate | I | X | HLA-B*07:02 | HLA- | Prostate |
| | | | | | | | 807:02 | |
| NRPLLANDL | 4216 | KLK4 | Human prostate | I | X | HLA-C*06:02 | HLA- | Prostate |
| | | | | | | HLA-C*07:02 | C01:02 | |
| SLQCVSLHL | 3456 | KLK2 | Human prostate | I | X | HLA-A*02:01 | HLA- | Prostate |
| | | | | | | | A02:01 | |
| TWIAPPLQV | 3784 | KLK3 | Human prostate | I | X | HLA-C*04:01 | HLA- | Prostate |
| | | | | | | | A02:01 | |
| VFQVSHSF | 3828 | KLK3 | Human prostate | I | X | HLA-C*07:02 | HLA- | Prostate |
| | | | | | | | A24:02 | |
| YSEKVTEFML | 3454 | KLK2 | Human prostate | I | X | HLA-A*01:01 | HLA- | Prostate |
| | | | | | | | A01:01 | |
| LLSHGAVIEV | 831 | ANKRD | CD165, | I | X | HLA-A*02:01 | HLA- | Breast |
| | | 30A | HCC1500, | | | | A02:01 | |
| | | | CAMA1 | | | | | |
| SIPTKALEL | 942 | ANKRD | B721, A375, | I | | | HLA- | Breast |
| | | 30A | CAMA1, | | | | C01:02 | |
| | | | HCC1500 | | | | | |
| SQYSGQLKV | 927 | ANKRD | B721 | I | | | HLA- | Breast |
| | | 30A | | | | | B13:02 | |
| SVPNKALEL | 941 | ANKRD | B721, A375 | I | X | HLA-C*04:01 | HLA- | Breast |
| | | 30A | | | | | C01:02 | |
| SLSKILDTV | 826 | ANKRD | CAMA1, | I | X | HLA-A*02:01 | HLA- | Breast |
| | | 30A | HCC1500 | | | | A02:01 | |
| SIPTKALEL | 942 | ANKRD | CAMA1 | I | X | HLA-C*04:01 | HLA- | Breast |
| | | 30A | | | | | C01:02 | |
| SLDQKLFQL | 827 | ANKRD | CAMA1, | I | X | HLA-A*02:01 | HLA- | Breast |
| | | 30A | HCC1500 | | | | A02:01 | |
| SVPNKALEL | 941 | ANKRD | HCC1500 | I | | | HLA- | Breast |
| | | 30A | | | | | C01:02 | |
| DSLFFLRGR | 6132 | PRAME | expi293 | I | | | HLA- | Lung squam.; |
| | | | | | | | A33:03 | Melanoma; |
| | | | | | | | | Ovarian; |
| | | | | | | | | Uterine |
| ELFSYLIEK | 6108 | PRAME | melanoma, | I | | | HLA- | Lung squam.; |
| | | | Human | | | | A03:01 | Melanoma; |
| | | | expi293 | | | | | Ovarian; |
| | | | | | | | | Uterine |
| FYDPEPILC | 6166 | PRAME | Jurkat | I | | | HLA- | Lung squam.; |
| | | | | | | | C04:01 | Melanoma; |
| | | | | | | | | Ovarian; |
| | | | | | | | | Uterine |
| SISALQSL | 6161 | PRAME | A375 | I | | | HLA- | Lung squam.; |
| | | | | | | | C03:04 | Melanoma; |
| | | | | | | | | Ovarian; |
| | | | | | | | | Uterine |
| ITDDQLLAL | 6088 | PRAME | A375 | I | | | HLA- | Lung squam.; |
| | | | | | | | A01:01 | Melanoma; |
| | | | | | | | | Ovarian; |
| | | | | | | | | Uterine |
| KRKKNVLRL | 6173 | PRAME | Human | I | | | HLA- | Lung squam.; |
| | | | melanoma | | | | C07:01 | Melanoma; |
| | | | | | | | | Ovarian; |
| | | | | | | | | Uterine |
| LQSLLQHLI | 6146 | PRAME | Human | I | | | HLA- | Lung squam.; |
| | | | melanoma | | | | B13:02 | Melanoma; |
| | | | | | | | | Ovarian; |
| | | | | | | | | Uterine |
| LSHIHASSY | 6152 | PRAME | Human | I | | | HLA- | Lung squam. |
| | | | melanoma | | | | B46:01 | Melanoma; |
| | | | | | | | | Ovarian; |
| | | | | | | | | Uterine |
| PYLGQMINL | 6120 | PRAME | melanoma, | I | | | HLA- | Lung squam.; |
| | | | Human | | | | A24:02 | Melanoma; |
| | | | A375, expi293 | | | | | Ovarian; |
| | | | | | | | | Uterine |
| QLLALLPSL | 6093 | PRAME | H1 | I | | | HLA- | Lung squam.; |
| | | | | | | | A02:01 | Melanoma; |
| | | | | | | | | Ovarian; |
| | | | | | | | | Uterine |
| SFYGNSISI | 6174 | PRAME | expi293 | I | | | HLA- | Lung squam.; |
| | | | | | | | C07:01 | Melanoma; |
| | | | | | | | | Ovarian; |
| | | | | | | | | Uterine |
| SLLQHLIGL | 6095 | PRAME | JEKO, ovarian | I | X | HLA-A*02:01 | HLA- | Lung squam.; |
| | | | Expi293, | | | | A02:01 | Melanoma; |
| | | | MDAPCa2b, | | | | | Ovarian; |
| | | | A375, buman | | | | | Uterine |
| | | | melanoma. | | | | | |
| | | | carcinoma, H1, | | | | | |
| | | | LNZ308, JY, | | | | | |
| | | | Hek293, HeLa, | | | | | |
| | | | Roja, THP1 | | | | | |
| SPSVSQLSVL | 6139 | PRAME | Human | I | | | HLA- | Lung squam.; |
| | | | melanoma, lung | | | | B07:02 | Melanoma; |
| | | | cancer, JY, H1, | | | | | Ovarian; |
| | | | A375 | | | | | Uterine |
| SPYLGQMINL | 6138 | PRAME | melanoma | I | | | HLA- | Lung squam.; |
| | | | | | | | B07:02 | Melanoma; |
| | | | | | | | | Ovarian; |
| | | | | | | | | Uterine |
| ISPRRLVEL | 6159 | PRAME | melanoma, | I | | | HLA- | Lung squam.; |
| | | | Human | | | | C01:02 | Melanoma; |
| | | | A375 | | | | | Ovarian; |
| | | | | | | | | Uterine |
| VLYPVPLESY | 6105 | PRAME | melanoma, lung | I | | | HLA- | Lung squam.; |
| | | | Human | | | | A03:01 | Melanoma; |
| | | | cancer | | | | | Ovarian; |
| | | | | | | | | Uterine |
| VSPEPLQAL | 6156 | PRAME | A375 | I | | | HLA- | Lung squam.; |
| | | | | | | | C01:02 | Melanoma; |
| | | | | | | | | Ovarian; |
| | | | | | | | | Uterine |
| YLHARLREL | 6140 | PRAME | melanoma, | I | | | HLA- | Lung squam.; |
| | | | Human | | | | B08:01 | Melanoma; |
| | | | expi293 | | | | | Ovarian; |
| | | | | | | | | Uterine |
| RLDQLLRHV | 6104 | PRAME | Human | I | | | HLA- | Lung squam.; |
| | | | melanoma | | | | A02:01 | Melanoma; |
| | | | | | | | | Ovarian; |
| | | | | | | | | Uterine |
| EVDPASNTY | 4638 | MAGEA | GBM, A375 | I | X | HLA-A*01:01 | HLA- | Lung squam. |
| | | 4 | | | | | A01:01 | |
| GVYDGREHTV | 4653 | MAGEA | Lung tumor, | I | X | HLA-A*02:01 | HLA- | Lung squam. |
| | | 4 | GBM, OvCa13, | | | | A02:01 | |
| | | | Breast tumor, | | | | | |
| | | | A375, | | | | | |
| | | | MSV000082648 | | | | | |
| KEVDPASNTY | 4640 | MAGEA | OvCa114, | I | | | HLA- | Lung squam. |
| | | 4 | A375, Lung | | | | A01:01 | |
| | | | tumor | | | | | |
| KVDELAHFL | 4648 | MAGEA | A375 | I | X | HLA-A*02:01 | HLA- | Lung squam. |
| | | 4 | | | | | A02:01 | |
| QIFPKTGL | 4692 | MAGEA | A375 | I | | | HLA- | Lung squam. |
| | | 4 | | | | | B08:01 | |
| QSPQGASAL | 4707 | MAGEA | A375 | I | | | HLA- | Lung squam. |
| | | 4 | | | | | C01:02 | |
| SALPTTISF | 4699 | MAGEA | Lung tumor | I | | | HLA- | Lung squam. |
| | | 4 | | | | | B46:01 | |
| TVYGEPRKL | 4722 | MAGEA | Human | I | | | HLA- | Lung squam. |
| | | 4 | melanoma. | | | | C07:01 | |
| | | | A375 | | | | | |
| VYGEPRKL | 4727 | MAGEA | A375 | I | | | HLA- | Lung squam. |
| | | 4 | | | | | C07:02 | |
| YPSLREAAL | 4682 | MAGEA | A375 | I | | | HLA- | Lung squam, |
| | | 4 | | | | | B07:02 | |
| ALLEEEEGV | 4644 | MAGEA | A375 | I | | | HLA- | Lung squam. |
| | | 4 | | | | | A02:01 | |
| KVLEHVVRV | 4643 | MAGEA | A375 | I | X | HLA-A*02:01 | HLA- | Lung squam. |
| | | 4 | | | | | A02:01 | |

TABLE 1C
Tumor Epitope Sequences

| | SEQ ID | | | HLA | MS | | Allele | |
|---|---|---|---|---|---|---|---|---|
| Epitope | NO: | Target | Sample | class | validation | Allele (MS) | (predicted) | Cancer |
| AYSEKVTEF | 3534 | KLK2 | MDAPCa2b | I | X | HLA-C*06:02 | HLA- | Prostate |
| | | | | | | | A24:02 | |
| HPEDTGQVF | 3988 | KLK3 | MDAPCa2b, | I | X | HLA-C*07:01 | HLA- | Prostate |
| | | | human prostate | | | HLA-C*04:01 | C07:01 | |
| HPEYNRPLL | 4143 | KLK4 | MDAPCa2b. | I | IX | HLA-C*07:01 | HLA- | Prostate |
| | | | human | | | HLA-B*07:02 | B07:02 | |
| | | | prostate, breast | | | | | |
| | | | cancer | | | | | |
| QRVPVSHSF | 3544 | KLK2 | MDAPCa2b, | I | IX | HLA-C*07:01 | HLA- | Prostate |
| | | | human prostate | | | HLA-C*07:02 | A24:02 | |
| LFHPEDTGQ | 3827 | KLK3 | Human | I | X | HLA-A*24:02 | HLA- | Prostate |
| VF | | | prostate | | | | A24:02 | |
| GYLQGLVSF | 4094 | KLK4 | Human | I | | HLA-A*24:02 | HLA- | Prostate |
| | | | prostate | | | | A24:02 | |
| IRNKSVILL | 3974 | KLK3 | Human | I | IX | HLA-C*06:02 | HLA- | Prostate |
| | | | prostate | | | HLA-C*07:02 | C07:01 | |
| KLQCVDLHV | 3740 | KLK3 | Human | I | | HLA-A*02:01 | HLA- | Prostate |
| | | | prostate | | | | A02:01 | |
| LLANGRMPT | 4029 | KLK4 | Human | I | X | HLA-A*02:01 | HLA- | Prostate |
| V | | | prostate | | | | A02:01 | |
| LRPGDDSTL | 3767 | KLK3 | Human | I | X | HLA-C*07:02 | HLA- | Prostate |
| | | | prostate | | | | C07:02 | |
| MPALPMVL | 3874 | KLK3 | Human | I | X | HLA-B*07:02 | HLA- | Prostate |
| | | | prostate | | | | B07:02 | |
| NRPLLANDL | 4216 | KLK4 | Human | I | X | HLA-C*06:02 | HLA- | Prostate |
| | | | prostate | | | HLA-C*07:02 | C01:02 | |
| SLQCVSLHL | 3456 | KLK2 | Human | I | X | HLA-A*02:01 | HLA- | Prostate |
| | | | prostate | | | | A02:01 | |
| TWIAPPLQV | 3784 | KLK3 | Human | I | X | HLA-C*04:01 | HLA- | Prostate |
| | | | prostate | | | | A02:01 | |
| VFQVSHSF | 3828 | KLK3 | Human | I | X | HLA-C*07:02 | HLA- | Prostate |
| | | | prostate | | | | A24:02 | |
| YSEKVTEFM | 3454 | KLK2 | Human | I | X | HLA-A*01:01 | HLA- | Prostate |
| L | | | prostate | | | | A01:01 | |
| LLSHGAVIE | 831 | ANKRD30A | CD165, | I | X | HLA-A*02:01 | HLA- | Breast |
| V | | | HCC1500, | | | | A02:01 | |
| | | | CAMA1 | | | | | |
| SVPNKALEL | 941 | ANKRD30A | B721, A375 | I | X | HLA-C*04:01 | HLA- | Breast |
| | | | | | | | C01:02 | |
| SLSKILDTV | 826 | ANKRD30A | CAMA1, | I | X | HLA-A*02:01 | HLA- | Breast |
| | | | HCC1500 | | | | A02:01 | |
| SIPTKALEL | 942 | ANKRD30A | CAMA1 | I | X | HLA-C*04:01 | HLA- | Breast |
| | | | | | | | C01:02 | |
| SLDQKLFQL | 827 | ANKRD30A | CAMA1, | I | | HLA-A*02:01 | HLA- | Breast |
| | | | HCC1500 | | | | A02:01 | |
| SLLQHLIGL | 6095 | PRAME | Expi293, | I | X | HLA-A*02:01 | HLA- | Lung squam.; |
| | | | MDAPCa2b, | | | | A02:01 | Melanoma; |
| | | | A375, human | | | | | Ovarian; Uterine |
| | | | melanoma, | | | | | |
| | | | JEKO, ovarian | | | | | |
| | | | carcinoma, H1, | | | | | |
| | | | LNZ308, JY | | | | | |
| | | | Hek293, HeLa, | | | | | |
| | | | Roja, THPI | | | | | |
| EVDPASNTY | 4638 | MAGEA4 | GBM, A375 | I | X | HLA-A*01:01 | HLA- | Lung squam. |
| | | | | | | | A01:01 | |
| GVYDGREHT | 4653 | MAGEA4 | 8 | I | X | HLA-A*02:01 | HLA- | Lung |
| V | | | GBM, | | | | A02:01 | squam. |
| | | | OvCa13, | | | | | |
| | | | Breast tumor, | | | | | |
| | | | Lung tumor, | | | | | |
| | | | A375, | | | | | |
| | | | MSV00008264 | | | | | |
| KVDELAHFL | 4648 | MAGEA4 | A375 | I | X | HLA-A*02:01 | HLA- | Lung squam. |
| | | | | | | | A02:01 | |
| KVLEHVVRV | 4643 | MAGEA4 | A375 | I | X | HLA-A*02:01 | HLA- | Lung |
| | | | | | | | A02:01 | squam. |

In aspects, provided herein are compositions comprising tissue-specific antigens. In some embodiments, the compositions comprise antigenic peptides, including tissue-specific antigens. In some embodiments, the tissue-specific antigens comprise tumor epitope sequence(s) as provided herein. In some embodiments, also provided herein are compositions comprising polynucleotides that code for the tissue-specific antigens.

In some embodiments, the size of the antigenic peptides provided herein comprise, but is not limited to, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino molecule residues, and any range derivable therein.

In some embodiments, the antigenic peptides are equal to or less than 50 amino acids. In some embodiments, the antigenic peptides are equal to about 20 to about 30 amino acids. A longer peptide can be designed in several ways. For example, when the HLA-binding regions are predicted or known, a longer peptide can consist of either: individual binding peptides with an extension of 0-10 amino acids toward the N- and C-terminus of each corresponding gene product. A longer peptide can also consist of a concatenation of some or all of the binding peptides with extended sequences for each.

The antigenic peptides and polypeptides can bind to or can be predicted to bind to an HLA protein. The antigenic peptide can have or can be predicted to have an IC50 of about less than 1000 nM, about less than 500 nM, about less than 250 nM, about less than 200 nM, about less than 150 nM, about less than 100 nM, or about less than 50 nM. In some embodiments, the antigenic peptides do not induce an autoimmune response and/or invoke immunological tolerance when administered to a subject.

### Identification of Tissue-Specific Antigen

In some aspects, the present disclosure provides methods of identifying tissue-specific antigens. In some embodiments, the tissue-specific antigen can be a tumor tissue specific epitope sequence.

In some embodiments, the methods provided herein comprise identifying an epitope sequence, which binds to or is predicted to bind to a protein encoded by a MHC allele expressed by a human subject, and is encoded by a tissue-specific antigen epitope gene that has an expression level in a tumor from a target tissue that is at least 2 fold greater than an expression level of the tissue-specific antigen epitope gene in each tissue of a plurality of non-target tissues that are different than the target tissue.

In some embodiments, the methods provided herein comprise identifying an epitope gene that has a higher expression level in a target tissue than in a non-target tissue. For example, the methods can comprise identifying an epitope gene that has a higher expression level in human pancreatic tissue than in human breast tissue, human lung tissue, or other human essential tissues. In some cases, the expression level in human pancreatic tissue can be at least 2 fold higher than in human breast tissue. In some embodiments, the step of identifying an epitope gene that has a higher expression level in a target tissue than in a non-target tissue comprises comparing expression level of the epitope gene in the target tissue versus in the non-target tissue. The comparison can be done by looking up the expression level of the epitope gene, at mRNA transcript or protein level, or both, profiled in compiled datasets, like TCGA (portal.gdc.cancer.gov/, last accessed September 2018), GTEX (gtexportal.org/home/, last accessed September 2018), GENT (medicalgenome.kribb.re.kr/GENT/, last accessed September 2018), The Human Protein Atlas (proteinatlas.org/, last accessed September 2018), Expression Atlas (ebi.ac.uk/gxa/home, last accessed September 2018), BioXpress (hive.biochemistry.gwu.edu/tools/bioxpress, last accessed September 2018), MERAV (merav.wi.mit.edu, last accessed September 2018), Global Cancer Map (globalcancermap.com/, last accessed September 2018), and CGAP (cgap.nci.nih.gov/, last accessed September 2018). Alternatively, the comparison can be done by experimental methods for assessing gene expression level, such as, but not limited to, techniques for assessing mRNA transcripts level like real time RT-PCR (real time-polymerase chain reaction), microarray, Northern blot, ISH (in situ hybridization), and RNA-seq (RNA sequencing), and techniques for assessing protein expression level like mass spectrometry, protein array, peptide array, immunostaining, and Western blot. Alternatively, the comparison can be done by: 1) first looking up profiled expression level in complied datasets, such as those discussed above, and 2) then experimentally validating the expression level in the tissues of interest.

In some embodiments, the methods provided herein comprise identifying a tumor epitope gene that has a higher expression level in a tumor from a target tissue than in each tissue of a plurality of non-target tissues that are different than the target tissue. For example, a prostate tumor is from prostate tissue, the methods provided herein can comprise identifying a tumor epitope gene that has a higher expression level in the prostate tumor than in each of a plurality of non-target tissues that are different than prostate, such as, but not limited to, brain, colon, lung, heart, and bone marrow.

In some embodiments, the methods provided herein comprise identifying a tumor epitope gene that has a higher expression level in a tumor from a target tissue than in an essential tissue. In some embodiments, the target tissue is a non-essential tissue. In some embodiments, an essential tissue comprises brain, colon, heart, bone marrow, or lung. In some embodiments, a non-essential tissue comprises thyroid, pancreas, adrenal, fallopian, prostate, breast, ovary, or cervix.

As provided herein, the tissue from which a tumor is derived from can be termed as target tissue, and other tissues or in some cases, essential tissues, can be termed as off-target tissues. In some embodiments, the methods provided herein comprise identifying tissue-specific antigen based on its absolute expression level in target tissue and off-target tissues. The expression level can be, in some cases, evaluated by RNA-seq reads. In some cases, the expression level can be expressed in units like “transcripts per million” (TPM) by which it can mean that the gene of interest has certain number of mRNA transcripts over one million total mRNA transcripts in a tissue of concern. In some embodiments, TPM can denominate protein coding mRNA transcripts, and non-protein coding genes are excluded for consideration. In some embodiments, the methods provided herein comprise identifying epitope sequence that is encoded by a tumor epitope gene that has an expression level of at least about 100 TPM in the target tissue, and has an expression level of at most about 5 TPM in off-target tissues. In some embodiments, the expression level of the epitope gene in the target tissue can be at least 10 TPM, at least 20 TPM, at least 30 TPM, at least 40 TPM, at least 50 TPM, at least 60 TPM, at least 70 TPM, at least 80 TPM, at least 90 TPM, at least 100 TPM, at least 110 TPM, at least 120 TPM, at least 130 TPM, at least 140 TPM, at least 150 TPM, at least 200 TPM, at least 300 TPM, at least 400 TPM, at least 500 TPM, at least 600 TPM, at least 700 TPM, at least 800 TPM, at least 1000 TPM, at least 2000 TPM, at least 3000 TPM, at least 5000 TPM, at least 10⁴ TPM, or greater. In some embodiments, the expression level of the epitope gene in off-target tissues can be at most 1000 TPM, at most 500 TPM, at most 100 TPM, at most 50 TPM, at most 20 TPM, at most 10 TPM, at most 9 TPM, at most 8 TPM, at most 7 TPM, at most 6 TPM, at most 5 TPM, at most 4 TPM, at most 3 TPM, at most 2 TPM, at most 1 TPM, at most 0.9 TPM, at most 0.8 TPM, at most 0.7 TPM, at most 0.6 TPM, at most 0.5 TPM, at most 0.4 TPM, at most 0.3 TPM, at most 0.2 TPM, at most 0.1 TPM, at most 0.050 TPM, at most 0.02 TPM, at most 0.010 TPM, at most 0.005 TPM, at most 0.002 TPM, at most 0.001 TPM, or lower.

In some embodiments, the methods comprise use of a computer algorithm to screen for tissue-specific epitope genes as provided herein. The computer algorithm can be constructed to access and examine available database containing expression data of a number of genes in different types of tissues. The computer algorithm can also be constructed to extract and compare the expression data as provided by various database, in order to identify genes of interest, e.g., tissue-specific genes, e.g., tissue-specific tumor epitope genes. In some embodiments, the computer algorithm can be constructed to report and display the screening results as can be viewed, extracted, and/or further processed by other computer algorithms. For example, the computer algorithm as provided herein can comprise different modules, among which there is one or more modules for identifying tissue-specific genes as provided herein, and there is also one or more modules for identifying epitope sequences from the identified tissue-specific genes.

In some embodiments, the methods provided herein comprise identifying an epitope sequence that can bind to or can be predicted to bind to a protein encoded by a MHC allele. In some embodiments, the MHC allele is expressed by a human subject. In some embodiments, the identification of epitope sequence that can bind to or can be predicted to bind to a protein encoded by a MHC allele expressed by a human subject is based on MHC binding affinity prediction, for example by one or more prediction algorithms. In some embodiments, the identification is based on experimental validation as will be discussed below. In some embodiments, the identification is based on both algorithm prediction and experimental validation. In some embodiments, the computer algorithms applicable to the subject matter include, but not limited to, evolutionary algorithms, artificial neural network-based algorithms, algorithms involving ant colony, hidden Markov models, support vector machines, and motif search, and any combination thereof. The computer algorithm can be based on convolutional neural networks (artificial intelligence or deep learning). The algorithms applicable the subject matter can be based on any appropriate prediction models. Non-limiting exemplary affinity prediction programs, tools, or online resources can include NetMHC, NetMHCIIpan, SVRMHC, DeepMHC, BiodMHC, sNebula, MHCPred, EpiToolKit, FRED, NNAlign, ProPred, HLA-DR4Pred, EpiTOP, CTLPred, TEPITOPEpan, SMM-align, ICES, GPS-MBA, EpiJen, PREDIVAC, EpicCapo, Epitopemap, ARB, EpiDOCK, HLArestrictor, MULTIPRED, MHCcluster, IMS (Immunogenetic Management Software), PAAQD, mHC2Pred, TEpredict, TepiTool, MMBPred, MHCMIR, HLAV3D, MHCBench, FDR4, LIGAP, MHC, HLAPred, HLA, POPISK, BiodMHC, MultiRTA, and MHC-BPS.

In some embodiments, the methods provided herein comprise identifying an epitope sequence that can bind to or can be predicted to bind to a protein encoded by a MHC allele and can be or can be predicted to be presented by an antigen-presenting cell. In some embodiments, the MHC allele is expressed by a human subject. In some embodiments, the antigen-presenting cell is a human antigen-presenting cell. The identification of affinity binding to MHC allele and presentation by APC can be based on prediction algorithms, experimental validation, or both.

### Therapeutic Methods and Compositions

Provided herein is a therapeutic composition comprising a peptide identified according to the method disclosed herein or a peptide as provided herein. Also provided herein is a method of providing an anti-tumor immunity in a mammal comprising administering to the mammal a polynucleic acid comprising a sequence encoding a peptide identified according to a method described herein. Provided herein is a method of providing an anti-tumor immunity in a mammal comprising administering to the mammal an effective amount of a peptide with a sequence of a peptide identified according to a method described herein. Provided herein is a method of providing an anti-tumor immunity in a mammal comprising administering to the mammal a cell comprising a peptide comprising the sequence of a peptide identified according to a method described herein. Provided herein is a method of providing an anti-tumor immunity in a mammal comprising administering to the mammal a cell comprising a polynucleic acid comprising a sequence encoding a peptide comprising the sequence of peptide identified according to a method described herein. In some embodiments, the cell presents the peptide as an HLA-peptide complex.

Provided herein is a therapeutic composition comprising a polynucleotide that comprises a sequence coding for a peptide identified according to the method disclosed herein or a peptide as provided herein. Also provided herein is a method of treating a disease or disorder in a subject, the method comprising administering to the subject a polynucleic acid comprising a sequence encoding a peptide identified according to a method described herein or a peptide as provided herein.

Provided herein is a method of treating a disease or disorder in a subject, the method comprising administering to the subject an effective amount of a peptide comprising the sequence of a peptide identified according to a method described herein or a peptide as provided herein. Provided herein is a method of treating a disease or disorder in a subject, the method comprising administering to the subject a cell comprising a peptide comprising the sequence of a peptide identified according to a method described herein or a peptide as provided herein. Provided herein is a method of treating a disease or disorder in a subject, the method comprising administering to the subject a cell comprising a polynucleic acid comprising a sequence encoding a peptide comprising the sequence of a peptide identified according to a method described herein or a peptide as provided herein. In some embodiments, wherein the disease or disorder is cancer. In some embodiments, the method further comprises administering an immune checkpoint inhibitor to the subject.

In some embodiments the present invention is directed to a therapeutic or pharmaceutical composition, e.g., a vaccine composition capable of raising a tissue-specific antigen response (e.g., a humoral or cell-mediated immune response). In some embodiments, the pharmaceutical composition comprises antigen therapeutic (e.g., peptides, polynucleotides, TCR, CAR, cells containing TCR or CAR, dendritic cell containing polypeptide, dendritic cell containing polynucleotide, antibody, etc.) described herein corresponding to tissue-specific antigen identified herein.

In some embodiments, a pharmaceutical composition provided herein comprises at least one antigen specific T cell comprising a T cell receptor (TCR) specific to at least one tissue-specific antigen peptide sequence provided herein. In some embodiments, the T cells are prepared by incubating FMS-like tyrosine kinase 3 receptor ligand (FLT3L) with a population of immune cells from a biological sample and incubating at least one T cell of the biological sample with an APC presenting the at least one tissue-specific antigen peptide sequence.

A person skilled in the art will be able to select antigenic therapeutics by testing, for example, the generation of T cells in vitro as well as their efficiency and overall presence, the proliferation, affinity and expansion of certain T cells for certain peptides, and the functionality of the T cells, e.g. by analyzing the IFN-γ production or tumor killing by T cells. The most efficient peptides can then combined as an immunogenic composition.

In some embodiments of the present invention the different antigenic peptides and/or polypeptides are selected so that one pharmaceutical composition comprises antigenic peptides and/or polypeptides capable of associating with different MHC molecules, such as different MHC class I molecule. In some embodiments, a pharmaceutical composition comprises antigenic peptides and/or polypeptides capable of associating with the most frequently occurring MHC class I molecules. Hence, immunogenic compositions described herein comprise different peptides capable of associating with at least 2, at least 3, or at least 4 MHC class I or class II molecules.

In some embodiments, a pharmaceutical composition described herein is capable of raising a specific cytotoxic T cells response, specific helper T cell response, or a B cell response.

In some embodiments, a pharmaceutical composition described herein can further comprise an adjuvant and/or a carrier. Examples of useful adjuvants and carriers are given herein below. Polypeptides and/or polynucleotides in the composition can be associated with a carrier such as e.g. a protein or an antigen-presenting cell such as e.g. a dendritic cell (DC) capable of presenting the peptide to a T cell or a B cell. In further embodiments, DC-binding peptides are used as carriers to target the antigenic peptides and polynucleotides encoding the tissue-specific antigen peptides to dendritic cells (Sioud et al. FASEB J 27: 3272-3283 (2013)).

In embodiments, the antigenic polypeptides or polynucleotides of the present disclosure can be provided as antigen presenting cells (e.g., dendritic cells) containing such polypeptides or polynucleotides. In other embodiments, such antigen presenting cells are used to stimulate T cells for use in patients.

In some embodiments, the antigen presenting cells are dendritic cells. In related embodiments, the dendritic cells are autologous dendritic cells that are pulsed with the antigenic peptide or nucleic acid. The antigenic peptide can be any suitable peptide that gives rise to an appropriate T cell response. T cell therapy using autologous dendritic cells pulsed with peptides from a tumor associated antigen is disclosed in Murphy et al. (1996) The Prostate 29, 371-380 and Tjua et al. (1997) The Prostate 32, 272-278. In some embodiments, the T cell is a CTL. In some embodiments, the T cell is a HTL.

Thus, one embodiment of the present invention provides a pharmaceutical composition containing at least one antigen presenting cell (e.g., a dendritic cell) that is pulsed or loaded with one or more antigenic polypeptides or polynucleotides described herein. In embodiments, such APCs are autologous (e.g., autologous dendritic cells). Alternatively, peripheral blood mononuclear cells (PBMCs) isolated from a patient can be loaded with antigenic peptides or polynucleotides ex vivo. In related embodiments, such APCs or PBMCs are injected back into the patient.

The polynucleotide of the present disclosure can be any suitable polynucleotide that is capable of transducing the dendritic cell, thus resulting in the presentation of a tissue-specific antigenic peptide and induction of immunity. In some embodiments, the polynucleotide can be naked DNA that is taken up by the cells by passive loading. In another embodiment, the polynucleotide is part of a delivery vehicle, for example, a liposome, virus like particle, plasmid, or expression vector. In another embodiment, the polynucleotide is delivered by a vector-free delivery system, for example, high performance electroporation and high-speed cell deformation). In embodiments, such antigen presenting cells (APCs) (e.g., dendritic cells) or peripheral blood mononuclear cells (PBMCs) are used to stimulate a T cell (e.g., an autologous T cell). In related embodiments, the T cell is a CTL. In other related embodiments, the T cell is an HTL. Such T cells are then injected into the patient. In some embodiments, CTL is injected into the patient. In some embodiments, HTL is injected into the patient. In some embodiments, both CTL and HTL are injected into the patient. Administration of either therapeutic can be performed simultaneously or sequentially and in any order.

In aspects, the present disclosure provides therapeutic compositions comprising immune cells, e.g., T cells that target tissue-specific antigens as provided herein, and methods of generating the compositions. In some embodiments, T cells are stimulated with one or more of the antigens described herein ex vivo. In some embodiments, the T cells that have been induced to recognize and target the tissue-specific antigens ex vivo are infused into the patient. In some embodiments, the infused T cells are from the patient himself/herself. In some embodiments, the infused T cells are from another subject.

In aspects, the present disclosure provides therapeutic compositions comprising TCRs that target the tissue-specific antigens provided herein and methods for generating the compositions. The TCRs provided herein can recognize one or more specific antigens. For instance, in some cases, the TCRs can be engineered to be bi-specific. In some cases, the TCRs can recognize one particular antigen specifically. In some cases, the TCRs can recognize one particular antigen specifically. In some embodiments, TCRs recognizing one or more of the tissue-specific antigens are identified a priori, for example, from a healthy donor. In some embodiments, the TCR(s) are knocked into T cells from the patient or other subject, e.g., the T cells are genetically modified to express the TCR(s) that are identified as recognizing one or more of the tissue-specific antigens. In some embodiments, the genetically modified T cells are infused into the patient.

In aspects, the present disclosure provides a method of discovering a TCR that recognizes an epitope, e.g., tissue-specific antigen. In some embodiments, the method comprises obtaining a T cell from a donor, and contacting the T cell with an antigen peptide in complex with an HLA of an APC from the donor. In some embodiments, the contacting can induce proliferation of the T cell. In some embodiments, the method further comprising determining a sequence of a TCR that recognizes the antigen peptide. In some embodiments, the donor is known to have zero or reduced immune tolerance to a tissue of origin of the antigen peptide. Without wishing to be bound to a certain theory, a subject, e.g., a human, can normally develop immune tolerance to proteins or peptides that are encoded by almost all normal genes (e.g., wild-type genes) of the subject in a healthy somatic tissue. However, in some cases, when a tissue of the same species is heterologous to the subject, the subject can have zero or low immune tolerance to proteins or peptides that are normally expressed in such tissue, for instance, a female human being can have low to none immune tolerance to human prostate-specific peptides (e.g., peptides specifically expressed in human prostate), and a male human being can have low to none immune tolerance to human ovary-specific peptides (e.g., peptides specifically expressed in human ovary). In some other cases, when a subject's immune system is deficient in developing immune tolerance to one or more of its own tissues, the subject can also have low to none immune tolerance to peptides specifically expressed in the one or more tissues, for instance, a type I diabetic subject can have autoimmunity against pancreas-specific peptide.

In some embodiments of the method of TCR discovery provided herein, the donor is a female subject, and the antigen peptide is specific to a tissue selected from the group consisting of: Bulbourethral gland, epididymis, penis, prostate, scrotum, seminal vesicle, testicle. In some embodiments, the donor is a female subject, and the antigen peptide is specific to prostate. In some embodiments, the donor is a male subject, and the antigen peptide is specific to a tissue selected from the group consisting of: Bartholin's gland, fallopian tube, ovary, Skene's gland, uterus, cervix, vagina, and any combination thereof. In some embodiments, the donor is a male subject, and the antigen peptide is specific to ovary. In some embodiments, the TCR discovered by contacting prostate-specific antigen peptide with T cells from female subject can be used for treatment of prostate cancer. In some embodiments, the TCR discovered by contacting ovary-specific antigen peptide with T cells from male subject can be used for treatment of ovarian cancer.

In some embodiments, the donor is a Type I diabetes patient, and the antigen peptide is specific to pancreas. In some embodiments, the TCR discovered by contacting pancreas-specific antigen peptide with T cells from Type I diabetic subject can be used for treatment of pancreas cancer. In some embodiments, the donor has auto-immune thyroid condition, and the antigen peptide is specific to thyroid. In some embodiments, the TCR discovered by contacting thyroid-specific antigen peptide with T cells from a subject with auto-immune thyroid condition can be used for treatment of thyroid cancer.

In aspects, the present disclosure provides therapeutic compositions comprising antibodies or functional part thereof that target the tissue-specific antigens provided herein and methods for generating the compositions. The antibodies provided herein can recognize one or more specific antigens. In some cases, the antibody as described herein can recognize one particular antigen specifically. In some embodiments, antibodies provided herein can find particular use for its specific binding to tissue-specific antigens that are expressed on cell surface. In some embodiments, antibodies provided herein can find particular use for its specific binding to tissue-specific antigens that are secreted outside of cells. In some embodiments, the antibodies can be isolated, recombinant, or purified for the therapeutic composition. Production of antibodies or functional part thereof can be carried out by techniques available to one skilled in the art. In some embodiments, antibodies can be produced by hybridomas or by such B cell culture. They can be harvested and for instance used for anticancer therapy. In some embodiments, they can be humanized before use in order to reduce side-effects.

The pharmaceutical compositions (e.g., immunogenic compositions) described herein for therapeutic treatment are intended for parenteral, topical, nasal, oral or local administration. In some embodiments, the pharmaceutical compositions described herein are administered parenterally, e.g., intravenously, subcutaneously, intradermally, or intramuscularly. In embodiments, the composition can be administered intratumorally. The compositions can be administered at the site of surgical excision to induce a local immune response to the tumor. In some embodiments, described herein are compositions for parenteral administration which comprise a solution of the antigenic peptides and immunogenic compositions are dissolved or suspended in an acceptable carrier, for example, an aqueous carrier. A variety of aqueous carriers can be used, e.g., water, buffered water, 0.9% saline, 0.3% glycine, hyaluronic acid and the like. These compositions can be sterilized by conventional, well known sterilization techniques, or can be sterile filtered. The resulting aqueous solutions can be packaged for use as is, or lyophilized, the lyophilized preparation being combined with a sterile solution prior to administration. The compositions can contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

The concentration of antigenic peptides and polynucleotides described herein in the pharmaceutical formulations can vary widely, i.e., from less than about 0.1%, usually at or at least about 2% to as much as 20% to 50% or more by weight, and will be selected by fluid volumes, viscosities, etc., according to the particular mode of administration selected.

The antigenic peptides and polynucleotides described herein can also be administered via liposomes, which target the peptides to a particular cells tissue, such as lymphoid tissue. Liposomes are also useful in increasing the half-life of the peptides. Liposomes include emulsions, foams, micelles, insoluble monolayers, liquid crystals, phospholipid dispersions, lamellar layers and the like. In these preparations the peptide to be delivered is incorporated as part of a liposome, alone or in conjunction with a molecule which binds to, e.g., a receptor prevalent among lymphoid cells, such as monoclonal antibodies which bind to the DEC205 antigen, or with other therapeutic or immunogenic compositions. Thus, liposomes filled with a desired peptide or polynucleotide described herein can be directed to the site of lymphoid cells, where the liposomes then deliver the selected therapeutic/immunogenic polypeptide/polynucleotide compositions. Liposomes can be formed from standard vesicle-forming lipids, which generally include neutral and negatively charged phospholipids and a sterol, for example, cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369.

For targeting to the immune cells, an antigenic polypeptides or polynucleotides to be incorporated into the liposome for cell surface determinants of the desired immune system cells. A liposome suspension containing a peptide can be administered intravenously, locally, topically, etc. in a dose which varies according to, inter alia, the manner of administration, the polypeptide or polynucleotide being delivered, and the stage of the disease being treated.

In some embodiments, antigenic polypeptides and polynucleotides are targeted to dendritic cells. In some embodiments, the antigenic polypeptides and polynucleotides are target to dendritic cells using the markers DEC205, XCR1, CD197, CD80, CD86, CD123, CD209, CD273, CD283, CD289, CD184, CD85h, CD85j, CD85k, CD85d, CD85g, CD85a, TSLP receptor, Clec9a or CD1a.

For solid compositions, conventional or nanoparticle nontoxic solid carriers can be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more antigenic polypeptides or polynucleotides described herein at a concentration of 25%-75%.

For aerosol administration, the antigenic polypeptides or polynucleotides can be supplied in finely divided form along with a surfactant and propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides can be employed. The surfactant can constitute 0.1%-20% by weight of the composition, or 0.25-5%. The balance of the composition can be propellant. A carrier can also be included as desired, as with, e.g., lecithin for intranasal delivery.

Additional methods for delivering the antigenic polynucleotides described herein are also known in the art. For instance, the nucleic acid can be delivered directly, as “naked DNA”. This approach is described, for instance, in Wolff et al., Science 247: 1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles.

For therapeutic or immunization purposes, mRNA encoding the antigenic peptides, or peptide binding agents can also be administered to the patient. In some embodiments, the mRNA is self-amplifying RNA. In a further embodiment, the self-amplifying RNA is a part of a synthetic lipid nanoparticle formulation (Geall et al., Proc Natl Acad Sci USA. 109: 14604-14609 (2012)).

The nucleic acids can also be delivered complexed to cationic compounds, such as cationic lipids. Lipid-mediated gene delivery methods are described, for instance, in WO 96/18372, WO 93/24640; Mannino & Gould-Fogerite, BioTechniques 6(7): 682-691 (1988); U.S. Pat. No. 5,279,833; WO 91/06309; and Felgner et al., Proc. Nat. Acad. Sci. USA 84: 7413-7414 (1987).

The antigenic peptides and polypeptides described herein can also be expressed by attenuated viruses, such as vaccinia or fowlpox. This approach involves the use of vaccinia virus as a vector to express nucleotide sequences that encode the peptide described herein. Upon introduction into an acutely or chronically infected host or into a noninfected host, the recombinant vaccinia virus expresses the immunogenic peptide, and thereby elicits a host CTL response. Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vectors useful for therapeutic administration or immunization of the peptides described herein will be apparent to those skilled in the art from the description herein.

Adjuvants are any substance whose admixture into the pharmaceutical composition increases or otherwise modifies the immune response to the therapeutic agent. Carriers are scaffold structures, for example a polypeptide or a polysaccharide, to which a tissue-specific antigenic polypeptide or polynucleotide, is capable of being associated. Optionally, adjuvants are conjugated covalently or non-covalently to the polypeptides or polynucleotides described herein.

The ability of an adjuvant to increase the immune response to an antigen is typically manifested by a significant increase in immune-mediated reaction, or reduction in disease symptoms. For example, an increase in humoral immunity can be manifested by a significant increase in the titer of antibodies raised to the antigen, and an increase in T cell activity can be manifested in increased cell proliferation, or cellular cytotoxicity, or cytokine secretion. An adjuvant can also alter an immune response, for example, by changing a primarily humoral or T helper 2 response into a primarily cellular, or T helper I response.

Suitable adjuvants are known in the art (see, WO 2015/095811) and include, but are not limited to poly(I:C), poly-ICLC, STING agonist, 1018 ISS, aluminium salts. Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206. Montanide ISA 50V, Montanide ISA-51, OK-432, OM-174, OM-197-MP-EC, ONTAK, PepTel®, vector system, PLG microparticles, resiquimod, SRL172, virosomes and other virus-like particles. YF-17D, VEGF trap, R848, beta-glucan, Pam3Cys, Pam3CSK4, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants also include incomplete Freund's or GM-CSF. Several immunological adjuvants (e.g., MF59) specific for dendritic cells and their preparation have been described previously (Dupuis M, et al., Cell Immunol. 1998: 186(1):18-27; Allison A C; Dev Biol Stand. 1998; 92:3-11) (Mosca et al. Frontiers in Bioscience, 2007; 12:4050-4060) (Gamvrellis et al. Immunol & Cell Biol. 2004; 82: 506-516). Also cytokines can be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, PGE1, PGE2, IL-1, IL-1b, IL-4, IL-6 and CD40L) (U.S. Pat. No. 5,849,589 incorporated herein by reference in its entirety) and acting as immunoadjuvants (e.g., IL-12) (Gabrilovich D I, et al., J Immunother Emphasis Tumor Immunol. 1996 (6):414-418).

CpG immunostimulatory oligonucleotides have also been reported to enhance the effects of adjuvants in a vaccine setting. Without being bound by theory, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell immunogenic pharmaceutical compositions, autologous cellular immunogenic pharmaceutical compositions and polysaccharide conjugates in both prophylactic and therapeutic immunogenic pharmaceutical compositions. Importantly, it enhances dendritic cell maturation and differentiation, resulting in enhanced activation of TH1 cells and strong cytotoxic T-lymphocyte (CTL) generation, even in the absence of CD4 T cell help. The TH I bias induced by TLR9 stimulation is maintained even in the presence of adjuvants such as alum or incomplete Freund's adjuvant (IFA) that normally promote a TH2 bias. CpG oligonucleotides show even greater adjuvant activity when formulated or co-administered with other adjuvants or in formulations such as microparticles, nano particles, lipid emulsions or similar formulations, which are especially necessary for inducing a strong response when the antigen is relatively weak. They also accelerate the immune response and enabled the antigen doses to be reduced with comparable antibody responses to the full-dose immunogenic pharmaceutical composition without CpG in some experiments (Arthur M. Krieg, Nature Reviews, Drug Discovery, 5, June 2006, 471-484). U.S. Pat. No. 6,406,705 B1 describes the combined use of CpG oligonucleotides, non-nucleic acid adjuvants and an antigen to induce an antigen-specific immune response. A commercially available CpG TLR9 antagonist is dSLIM (double Stem Loop Immunomodulator) by Mologen (Berlin, GERMANY), which is a component of the pharmaceutical composition described herein. Other TLR binding molecules such as RNA binding TLR 7, TLR 8 and/or TLR 9 can also be used.

Other examples of useful adjuvants include, but are not limited to, chemically modified CpGs (e.g. CpR, Idera), polyTCLC, Poly(I:C)(e.g. polyi:CI2U), non-CpG bacterial DNA or RNA, ssRNA40 for TLR8, as well as immunoactive small molecules and antibodies such as cyclophosphamide, sunitinib, bevacizumab, celebrex, NCX-4016, sildenafil, tadalafil, vardenafil, sorafinib, XL-999. CP-547632, pazopanib, AZD2171, ipilimumab, tremelimumab, and SC58175, which can act therapeutically and/or as an adjuvant. The amounts and concentrations of adjuvants and additives useful in the context of the present invention can readily be determined by the skilled artisan without undue experimentation. Additional adjuvants include colony-stimulating factors, such as Granulocyte Macrophage Colony Stimulating Factor (GM-CSF, sargramostim).

In some embodiments, a pharmaceutical composition according to the present invention comprises more than one different adjuvants. Furthermore, the invention encompasses a therapeutic composition comprising any adjuvant substance including any of the above or combinations thereof. It is also contemplated that the antigenic therapeutic (e.g., a humoral or cell-mediated immune response). In some embodiments, the pharmaceutical composition comprises tissue-specific antigen therapeutics (e.g., peptides, polynucleotides, TCR, CAR, cells containing TCR or CAR, dendritic cell containing polypeptide, dendritic cell containing polynucleotide, antibody, etc.) and the adjuvant can be administered separately in any appropriate sequence.

A carrier can be present independently of an adjuvant. The function of a carrier can for example be to increase the molecular weight of in particular mutant in order to increase their activity or immunogenicity, to confer stability, to increase the biological activity, or to increase serum half-life. Furthermore, a carrier can aid presenting peptides to T cells. The carrier can be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. In some embodiments, the carrier comprises a human fibronection type III domain (Koide et al. Methods Enzymol. 2012; 503:135-56). For immunization of humans, the carrier must be a physiologically acceptable carrier acceptable to humans and safe. However, tetanus toxoid and/or diptheria toxoid are suitable carriers In some embodiments of the invention. Alternatively, the carrier can be dextrans for example sepharose.

In some embodiments, the polypeptides can be synthesized as multiply linked peptides as an alternative to coupling a polypeptide to a carrier to increase immunogenicity. Such molecules are also known as multiple antigenic peptides (MAPS).

Tissue-specific antigens as described herein that induce an immune response can be used as a composition when combined with an acceptable carrier or excipient. Such compositions are useful for in vitro or in vivo analysis or for administration to a subject in vivo or ex vivo for treating a subject with a disease.

Thus, pharmaceutical compositions can include, in addition to active ingredient, a pharmaceutically acceptable excipient, carrier, buffer, stabilizer or other materials well known to those skilled in the art. Such materials should be non-toxic and should not interfere with the efficacy of the active ingredient. The precise nature of the carrier or other material will depend on the route of administration.

Pharmaceutical formulations comprising a protein of interest, e.g., a tissue-specific antigen described herein, can be prepared for storage by mixing the antigen having the desired degree of purity with optional physiologically acceptable carriers, excipients or stabilizers (Remington's Pharmaceutical Sciences 16th edition, Oslo, A. Ed. (1980)), in the form of lyophilized formulations or aqueous solutions. Acceptable carriers, excipients, or stabilizers are those that are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

Acceptable carriers are physiologically acceptable to the administered patient and retain the therapeutic properties of the compounds with/in which it is administered. Acceptable carriers and their formulations are generally described in, for example, Remington' pharmaceutical Sciences (18th Edition, ed. A. Gennaro, Mack Publishing Co., Easton, PA 1990). One exemplary carrier is physiological saline. A pharmaceutically acceptable carrier is a pharmaceutically acceptable material, composition or vehicle, such as a liquid or solid filler, diluent, excipient, solvent or encapsulating material, involved in carrying or transporting the subject compounds from the administration site of one organ, or portion of the body, to another organ, or portion of the body, or in an in vitro assay system. Acceptable carriers are compatible with the other ingredients of the formulation and not injurious to a subject to whom it is administered. Nor should an acceptable carrier alter the specific activity of the tissue-specific antigens.

In one aspect, provided herein are pharmaceutically acceptable or physiologically acceptable compositions including solvents (aqueous or non-aqueous), solutions, emulsions, dispersion media, coatings, isotonic and absorption promoting or delaying agents, compatible with pharmaceutical administration. Pharmaceutical compositions or pharmaceutical formulations therefore refer to a composition suitable for pharmaceutical use in a subject. The pharmaceutical compositions and formulations include an amount of a tissue-specific antigen as provided herein (or polynucleotide encoding the tissue-specific antigen) and a pharmaceutically or physiologically acceptable carrier. Compositions can be formulated to be compatible with a particular route of administration (i.e., systemic or local). Thus, compositions include carriers, diluents, or excipients suitable for administration by various routes.

In some embodiments, a composition further comprises an acceptable additive in order to improve the stability of the tissue-specific antigen in the composition and/or to control the release rate of the composition. Acceptable additives do not alter the specific activity of the tissue-specific antigens. Exemplary acceptable additives include, but are not limited to, a sugar such as mannitol, sorbitol, glucose, xylitol, trehalose, sorbose, sucrose, galactose, dextran, dextrose, fructose, lactose and mixtures thereof. Acceptable additives can be combined with acceptable carriers and/or excipients such as dextrose. Alternatively, exemplary acceptable additives include, but are not limited to, a surfactant such as polysorbate 20 or polysorbate 80 to increase stability of the peptide and decrease gelling of the solution. The surfactant can be added to the composition in an amount of 0.01% to 5% of the solution. Addition of such acceptable additives increases the stability and half-life of the composition in storage.

The pharmaceutical composition can be administered, for example, by injection. Compositions for injection include aqueous solutions (where water soluble) or dispersions and sterile powders for the extemporaneous preparation of sterile injectable solutions or dispersion. For intravenous administration, suitable carriers include physiological saline, bacteriostatic water, or phosphate buffered saline (PBS). The carrier can be a solvent or dispersion medium containing, for example, water, ethanol, polyol (for example, glycerol, propylene glycol, and liquid polyethylene glycol, and the like), and suitable mixtures thereof. Fluidity can be maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid and thimerosal. Isotonic agents, for example, sugars, polyalcohols such as manitol, sorbitol, and sodium chloride can be included in the composition. The resulting solutions can be packaged for use as is, or lyophilized: the lyophilized preparation can later be combined with a sterile solution prior to administration. For intravenous, injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as needed. Sterile injectable solutions can be prepared by incorporating an active ingredient in the required amount in an appropriate solvent with one or a combination of ingredients enumerated above, as required, followed by filtered sterilization. Generally, dispersions are prepared by incorporating the active ingredient into a sterile vehicle which contains a basic dispersion medium and the required other ingredients from those enumerated above. In the case of sterile powders for the preparation of sterile injectable solutions, the preferred methods of preparation are vacuum drying and freeze drying which yields a powder of the active ingredient plus any additional desired ingredient from a previously sterile-filtered solution thereof.

Compositions can be conventionally administered intravenously, such as by injection of a unit dose, for example. For injection, an active ingredient can be in the form of a parenterally acceptable aqueous solution which is substantially pyrogen-free and has suitable pH, isotonicity and stability. One can prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required. Additionally, compositions can be administered via aerosolization.

In some embodiments, the composition is lyophilized, for example, to increase shelf-life in storage. When the compositions are considered for use in medicaments or any of the methods provided herein, it is contemplated that the composition can be substantially free of pyrogens such that the composition will not cause an inflammatory reaction or an unsafe allergic reaction when administered to a human patient. Testing compositions for pyrogens and preparing compositions substantially free of pyrogens are well understood to one or ordinary skill of the art and can be accomplished using commercially available kits.

Acceptable carriers can contain a compound that stabilizes, increases or delays absorption, or increases or delays clearance. Such compounds include, for example, carbohydrates, such as glucose, sucrose, or dextrans; low molecular weight proteins: compositions that reduce the clearance or hydrolysis of peptides; or excipients or other stabilizers and/or buffers. Agents that delay absorption include, for example, aluminum monostearate and gelatin. Detergents can also be used to stabilize or to increase or decrease the absorption of the pharmaceutical composition, including liposomal carriers. To protect from digestion the compound can be complexed with a composition to render it resistant to acidic and enzymatic hydrolysis, or the compound can be complexed in an appropriately resistant carrier such as a liposome.

The compositions can be administered in a manner compatible with the dosage formulation, and in a therapeutically effective amount. The quantity to be administered depends on the subject to be treated, capacity of the subject's immune system to utilize the active ingredient, and degree of binding capacity desired. Precise amounts of active ingredient required to be administered depend on the judgment of the practitioner and are peculiar to each individual. Suitable regimes for initial administration and booster shots arc also variable, but are typified by an initial administration followed by repeated doses at one or more hour intervals by a subsequent injection or other administration. Alternatively, continuous intravenous infusions sufficient to maintain concentrations in the blood are contemplated.

Peptide-based immunogenic pharmaceutical compositions can be formulated using any of the well-known techniques, carriers, and excipients as suitable and as understood in the art. The polypeptides can be a cocktail of multiple polypeptides containing the same sequence, or a cocktail of multiple copies of different polypeptides. The peptides can be modified, such as for example by lipidation, or attachment to a carrier protein. Lipidation can be the covalent attachment of a lipid group to a polypeptide. Lipidated peptides, or lipidated polypeptides, can stabilize structures and can enhance efficacy of the treatment.

Lipidation can be classified into several different types, such as N-myristoylation, palmitoylation, GPI-anchor addition, prenylation, and several additional types of modifications. N-myristoylation is the covalent attachment of myristate, a C14 saturated acid, to a glycine residue. Palmitoylation is thioester linkage of long-chain fatty acids (C16) to cysteine residues. GPI-anchor addition is glycosyl-phosphatidylinositol (GPI) linkage via amide bond. Prenylation is the thioether linkage of an isoprenoid lipid (e.g. farnesyl (C-15), geranylgeranyl (C-20)) to cysteine residues. Additional types of modifications can include attachment of S-diacylglycerol by a sulfur atom of cysteines, O-octanoyl conjugation via serine or threonine residues, S-archaeol conjugation to cysteine residues, and cholesterol attachment.

Fatty acids for generating a lipidated peptides can include C2 to C30 saturated, monounsaturated, or polyunsaturated fatty acyl groups. Exemplary fatty acids can include palmitoyl, myristoyl, stearoyl and decanoyl groups. In some instances, a lipid moiety that has adjuvant property is attached to a polypeptide of interest to elicit or enhance immunogenicity in the absence of an extrinsic adjuvant. A lipidated peptide or lipopeptide can be referred to as a self-adjuvant lipopeptide. Any of the fatty acids described above and elsewhere herein can elicit or enhance immunogenicity of a polypeptide of interest. A fatty acid that can elicit or enhance immunogenicity can include palmitoyl, myristoyl, stearoyl, lauroyl, octanoyl, and decanoyl groups.

Polypeptides such as naked peptides or lipidated peptides can be incorporated into a liposome. Sometimes, lipidated peptides can be incorporated into a liposome. For example, the lipid portion of the lipidated peptide can spontaneously integrate into the lipid bilayer of a liposome. Thus, a lipopeptide can be presented on the “surface” of a liposome.

Exemplary liposomes suitable for incorporation in the formulations include, and are not limited to, multilamellar vesicles (MLV), oligolamellar vesicles (OLV), unilamellar vesicles (UV), small unilamellar vesicles (SUV), medium-sized unilamellar vesicles (MUV), large unilamellar vesicles (LUV), giant unilamellar vesicles (GUV), multivesicular vesicles (MVV), single or oligolamellar vesicles made by reverse-phase evaporation method (REV), multilamellar vesicles made by the reverse-phase evaporation method (MLV-REV), stable plurilamellar vesicles (SPLV), frozen and thawed MLV (FATMLV), vesicles prepared by extrusion methods (VET), vesicles prepared by French press (FPV), vesicles prepared by fusion (FUV), dehydration-rehydration vesicles (DRV), and bubblesomes (BSV).

Depending on the method of preparation, liposomes can be unilamellar or multilamellar, and can vary in size with diameters ranging from about 0.02 μm to greater than about 10 μm. Liposomes can adsorb many types of cells and then release an incorporated agent (e.g., a peptide described herein). In some cases, the liposomes fuse with the target cell, whereby the contents of the liposome then empty into the target cell. A liposome can be endocytosed by cells that are phagocytic. Endocytosis can be followed by intralysosomal degradation of liposomal lipids and release of the encapsulated agents.

The liposomes provided herein can also comprise carrier lipids. In some embodiments the carrier lipids are phospholipids. Carrier lipids capable of forming liposomes include, but are not limited to dipalmitoylphosphatidylcholine (DPPC), phosphatidylcholine (PC; lecithin), phosphatidic acid (PA), phosphatidylglycerol (PG), phosphatidylethanolamine (PE), phosphatidylserine (PS). Other suitable phospholipids further include distearoylphosphatidylcholine (DSPC), dimyristoylphosphatidylcholine (DMPC), dipalmitoylphosphatidyglycerol (DPPG), distearoylphosphatidyglycerol (DSPG), dimyristoylphosphatidylglycerol (DMPG), dipalmitoylphosphatidic acid (DPPA); dimyristoylphosphatidic acid (DMPA), distearoylphosphatidic acid (DSPA), dipalmitoylphosphatidylserine (DPPS), dimyristoylphosphatidylserine (DMPS), distearoylphosphatidylserine (DSPS), dipalmitoylphosphatidyethanolamine (DPPE), dimyristoylphosphatidylethanolamine (DMPE), distearoylphosphatidylethanolamine (DSPE) and the like, or combinations thereof. In some embodiments, the liposomes further comprise a sterol (e.g., cholesterol) which modulates liposome formation. The carrier lipids can be any known non-phosphate polar lipids.

A pharmaceutical composition can be encapsulated within liposomes using well-known technology. Biodegradable microspheres can also be employed as carriers for the pharmaceutical compositions of this invention.

The pharmaceutical composition can be administered in liposomes or microspheres (or microparticles). Methods for preparing liposomes and microspheres for administration to a patient are well known to those of skill in the art. Essentially, material is dissolved in an aqueous solution, the appropriate phospholipids and lipids added, along with surfactants if required, and the material dialyzed or sonicated, as necessary.

Microspheres formed of polymers or proteins are well known to those skilled in the art, and can be tailored for passage through the gastrointestinal tract directly into the blood stream. Alternatively, the compound can be incorporated and the microspheres, or composite of microspheres, implanted for slow release over a period of time ranging from days to months.

A polypeptide can also be attached to a carrier protein for delivery. The carrier protein can be an immunogenic carrier element and can be attached by any recombinant technology. Exemplary carrier proteins include Mariculture keyhole limpet hemocyanin (mcKLH), PEGylated mcKLH, Blue Carrier* Proteins, bovine serum albumin (BSA), cationized BSA, ovalbumin, and bacterial proteins such as tetanus toxoid (TT).

A polypeptide can also be prepared as multiple antigenic peptides (MAPs). Peptides may be attached at the N-terminus or the C-terminus to small non-immunogenic cores. Peptides built upon this core can offer highly localized peptide density. The core can be a dendritic core residue or matrix composed of bifunctional units. Suitable core molecules for constructing MAPs can include ammonia, ethylenediamine, aspartic acid, glutamic acid, and lysine. For example, a lysine core molecule can be attached via peptide bonds through each of its amino groups to two additional lysines.

A polypeptide can be chemically synthesized, or recombinantly expressed in a cell system or a cell-free system. A peptide can be synthesized, such as by a liquid-phase synthesis, a solid-phase synthesis, or by microwave assisted peptide synthesis. A polypeptide can be modified, such as for example, by acylation, alkylation, amidation, arginylation, polyglutamylation, polyglycylation, butyrylation, gamma-carboxylation, glycosylation, malonylation, hydroxylation, iodination, nucleotide addition (e.g. ADP-ribosylation), oxidation, phosphorylation, adenylylation, propionylation, S-glutathionylation, S-nitrosylation, succinylation, sulfation, glycation, palmitoylation, myristoylation, isoprenylation or prenylation (e.g. farnesylation or geranylgeranylation), glypiation, lipoylation, attachement of flavin moiety (e.g. FMN or FAD), attachment of heme C, phosphopantetheinylation, retinylidene Schiff base formation, diphthamide formation, ethanolamine phosphoglycerol attachment, hypusine formuation, biotinylation, pegylation, ISGylation, SUMUylation, ubiquitination, Neddylation, Pupylation, citrullination, deamidation, eliminylation, carbamylation, or a combination thereof.

After generation of a polypeptide, the polypeptide can be subjected to one or more rounds of purification steps to remove impurities. The purification step can be a chromatographic step utilizing separation methods such as affinity-based, size-exclusion based, ion-exchange based, or the like. In some cases, the polypeptide is at most 30%, 40%, 50%, 60%, 70%, 80%, 90%, 95%, 99%, 99.9%, or 100% pure or without the presence of impurities. In some cases, the polypeptide is at least 30%, 40%, 50%, 60%, 70%, 80%, 90%, 95%, 99%, 99.9%, or 100% pure or without the presence of impurities.

A polypeptide can include natural amino acids, unnatural amino acids, or a combination thereof. An amino acid residue can refer to a molecule containing both an amino group and a carboxyl group. Suitable amino acids include, without limitation, both the D- and L-isomers of the naturally-occurring amino acids, as well as non-naturally occurring amino acids prepared by organic synthesis or other metabolic routes. The term amino acid, as used herein, includes, without limitation, α-amino acids, natural amino acids, non-natural amino acids, and amino acid analogs.

The term “α-amino acid” can refer to a molecule containing both an amino group and a carboxyl group bound to a carbon which is designated the a-carbon.

The term “β-amino acid” can refer to a molecule containing both an amino group and a carboxyl group in a p configuration.

“Naturally occurring amino acid” can refer to anyone of the twenty amino acids commonly found in peptides synthesized in nature, and known by the one letter abbreviations A, R, N, C, D, Q, E, G, H, I, L, K, M, F, P, S, T, W, Y and V. A table showing a summary of the properties of natural amino acids can be found, e.g., in U.S. Patent Application Publication No. 20130123169, which is herein incorporated by reference.

A peptide provided herein can comprise one or more hydrophobic, polar, or charged amino acids. “Hydrophobic amino acids” include small hydrophobic amino acids and large hydrophobic amino acids. “Small hydrophobic amino acid” can be glycine, alanine, proline, and analogs thereof. “Large hydrophobic amino acids” can be valine, leucine, isoleucine, phenylalanine, methionine, tryptophan, and analogs thereof. “Polar amino acids” can be serine, threonine, asparagine, glutamine, cysteine, tyrosine, and analogs thereof. “Charged amino acids” can be lysine, arginine, histidine, aspartate, glutamate, and analogs thereof.

A peptide provided herein can comprise one or more amino acid analogs. An “amino acid analog” can be a molecule which is structurally similar to an amino acid and which can be substituted for an amino acid in the formation of a peptidomimetic macrocycle Amino acid analogs include, without limitation, β-amino acids and amino acids where the amino or carboxy group is substituted by a similarly reactive group (e.g., substitution of the primary amine with a secondary or tertiary amine, or substitution of the carboxy group with an ester).

A peptide provided herein can comprises one or more non-natural amino acids. A “non-natural amino acid” can be an amino acid which is not one of the twenty amino acids commonly found in peptides synthesized in nature, and known by the one letter abbreviations A, R, N, C, D, Q, E, G, H, I, L, K, M, F, P, S, T, W, Y and V. Non-natural amino acids or amino acid analogs include structures disclosed, e.g., in U.S. Patent Application Publication No. 20130123169, which is herein incorporated by reference.

Amino acid analogs can include β-amino acid analogs. Examples of β-amino acid analogs and analogs of alanine, valine, glycine, leucine, arginine, lysine, aspartic acids, glutamic acids, cysteine, methionine, phenylalanine, tyrosine, proline, serine, threonine, and tryptophan can include structures disclosed, e.g., in U.S. Patent Application Publication No. 20130123169, which is herein incorporated by reference.

Amino acid analogs can be racemic. In some instances, the D isomer of the amino acid analog is used. In some cases, the L isomer of the amino acid analog is used. In some instances, the amino acid analog comprises chiral centers that are in the R or S configuration. Sometimes, the amino group(s) of a β-amino acid analog is substituted with a protecting group, e.g., tert-butyloxycarbonyl (BOC group), 9-fluorenylmethyloxycarbonyl (FMOC), tosyl, and the like. Sometimes, the carboxylic acid functional group of a P-amino acid analog is protected, e.g., as its ester derivative. In some cases, the salt of the amino acid analog is used.

A “non-essential” amino acid residue can be a residue that can be altered from the wild-type sequence of a polypeptide without abolishing or substantially altering its essential biological or biochemical activity (e.g., receptor binding or activation). An “essential” amino acid residue can be a residue that, when altered from the wild-type sequence of the polypeptide, results in abolishing or substantially abolishing the polypeptide's essential biological or biochemical activity.

A “conservative amino acid substitution” can be one in which the amino acid residue is replaced with an amino acid residue having a similar side chain. Families of amino acid residues having similar side chains have been defined in the art. These families can include amino acids with basic side chains (e.g., K, R, H), acidic side chains (e.g., D, E), uncharged polar side chains (e.g., G. N, Q, S, T, Y, C), nonpolar side chains (e.g., A, V, L, I, P, F, M, W), beta-branched side chains (e.g., T, V,) and aromatic side chains (e.g., Y, F, W, H). Thus, a predicted nonessential amino acid residue in a polypeptide, for example, can be replaced with another amino acid residue from the same side chain family. Other examples of acceptable substitutions can be substitutions based on isosteric considerations (e.g. norleucine for methionine) or other properties (e.g. 2-thienylalanine for phenylalanine, or 6-Cl-tryptophan for tryptophan).

Nucleic acid-based immunogenic pharmaceutical compositions can also be administered to a subject. Nucleic acid-based immunogenic pharmaceutical compositions can be formulated using any of the well-known techniques, carriers, and excipients as suitable and as understood in the art. The nucleic acid can be DNA, genomic DNA or cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods. The immunogenic pharmaceutical composition can be a DNA-based immunogenic pharmaceutical composition, an RNA-based immunogenic pharmaceutical composition, a hybrid DNA/RNA based immunogenic pharmaceutical composition, or a hybrid nucleic acid/peptide based immunogenic pharmaceutical composition. The peptide can be a peptide derived from a peptide in Table 1A, Table 1B, Table 1C or Table 2, a peptide that has a sequence that is at least 40%, 50%, 60%, 70%, 80%, 90%, 95%, or more in sequence homology to a peptide in Table 1A, Table 1B, Table 1C or Table 2, or a peptide that has a sequence that is at most 40%, 50%, 60%, 70%, 80%, 90%, 95%, or less in sequence homology to a peptide in Table 1A, Table 1B, Table 1C or Table 2.

A nucleic acid described herein can contain phosphodiester bonds, although in some cases, as outlined below (for example in the construction of primers and probes such as label probes), nucleic acid analogs are included that can have alternate backbones, comprising, for example, phosphoramide, phosphorothioate, O-methylphosphoroamidite linkages, and peptide nucleic acid backbones and linkages. Other analog nucleic acids include those with bicyclic structures including locked nucleic acids, positive backbones and non-ribose backbones. Nucleic acids containing one or more carbocyclic sugars are also included within the definition of nucleic acids. Locked nucleic acids (LNAs) are also included within the definition of nucleic acid analogs. LNAs are a class of nucleic acid analogues in which the ribose ring is “locked” by a methylene bridge connecting the 2′-0 atom with the 4′-C atom. These modifications of the ribose-phosphate backbone can be done to increase the stability and half-life of such molecules in physiological environments. For example, PNA:DNA and LNA-DNA hybrids can exhibit higher stability and thus can be used in some embodiments. The nucleic acids can be single stranded or double stranded, as specified, or contain portions of both double stranded or single stranded sequence. Depending on the application, the nucleic acids can be DNA (including, e.g., genomic DNA, mitochondrial DNA, and cDNA), RNA (including, e.g., mRNA and rRNA) or a hybrid, where the nucleic acid contains any combination of deoxyribose- and ribo-nucleotides, and any combination of bases, including uracil, adenine, thymine, cytosine, guanine, inosine, xathanine hypoxathanine, isocytosine, isoguanine, etc.

A nucleic acid-based immunogenic pharmaceutical compositions can be in the form of a vector. A vector can be a circular plasmid or a linear nucleic acid. A circular plasmid or linear nucleic acid can be capable of directing expression of a particular nucleotide sequence in an appropriate subject cell. A vector can have a promoter operably linked to the polypeptide-encoding nucleotide sequence, which can be operably linked to termination signals. A vector can contain sequences required for proper translation of the nucleotide sequence. The vector comprising the nucleotide sequence of interest can be chimeric, meaning that at least one of its components can be heterologous with respect to at least one of its other components. The expression of the nucleotide sequence in an expression cassette can be under the control of a constitutive promoter or of an inducible promoter, which can initiate transcription only when the host cell is exposed to some particular internal or external stimulus.

The vector can be a plasmid. A plasmid can be useful for transfecting cells with nucleic acid encoding the polypeptide, and the transformed host cells can be cultured and maintained under conditions wherein expression of the polypeptide takes place.

A plasmid can comprise a nucleic acid sequence that encodes one or more of the various polypeptides disclosed herein. A single plasmid can contain coding sequence for a single polypeptide, or coding sequence for more than one polypeptide. Sometimes, the plasmid can further comprise coding sequence that encodes an adjuvant, such as an immune stimulating molecule, such as a cytokine.

A plasmid can further comprise an initiation codon, which can be upstream of the coding sequence, and a stop codon, which can be downstream of the coding sequence. The initiation and termination codon can be in frame with the coding sequence. A plasmid can also comprise a promoter that is operably linked to the coding sequence, and an enhancer upstream of the coding sequence. The enhancer can be human actin, human myosin, human hemoglobin, human muscle creatine or a viral enhancer such as one from CMV, FMDV, RSV or EBV.

A plasmid can also comprise a mammalian origin of replication in order to maintain the plasmid extrachromosomally and produce multiple copies of the plasmid in a cell. A plasmid can also comprise a regulatory sequence, which can be well suited for gene expression in a cell into which the plasmid is administered. The coding sequence can comprise a codon that can allow more efficient transcription of the coding sequence in the host cell.

The nucleic acid based immunogenic pharmaceutical compositions can also be a linear nucleic acid immunogenic pharmaceutical composition, or linear expression cassette, that is capable of being efficiently delivered to a subject via electroporation and expressing one or more polypeptides disclosed herein.

Cell-based immunogenic pharmaceutical compositions can also be administered to a subject. For example, an antigen presenting cell (APC) based immunogenic pharmaceutical composition can be formulated using any of the well-known techniques, carriers, and excipients as suitable and as understood in the art. APCs include monocytes, monocyte-derived cells, macrophages, and dendritic cells. Sometimes, an APC based immunogenic pharmaceutical composition can be a dendritic cell-based immunogenic pharmaceutical composition.

A dendritic cell-based immunogenic pharmaceutical composition can be prepared by any methods well known in the art. In some cases, dendritic cell-based immunogenic pharmaceutical compositions can be prepared through an ex vivo or in vivo method. The ex vivo method can comprise the use of autologous DCs pulsed ex vivo with the polypeptides described herein, to activate or load the DCs prior to administration into the patient. The in vivo method can comprise targeting specific DC receptors using antibodies coupled with the polypeptides described herein. The DC-based immunogenic pharmaceutical composition can further comprise DC activators such as TLR3, TLR-7-8, and CD40 agonists. The DC-based immunogenic pharmaceutical composition can further comprise adjuvants, and a pharmaceutically acceptable carrier.

An adjuvant can be used to enhance the immune response (humoral and/or cellular) elicited in a patient receiving the immunogenic pharmaceutical composition. Sometimes, adjuvants can elicit a Th1-type response. Other times, adjuvants can elicit a Th2-type response. A Th1-type response can be characterized by the production of cytokines such as IFN-γ as opposed to a Th2-type response which can be characterized by the production of cytokines such as IL-4, IL-5 and IL-10.

In some aspects, lipid-based adjuvants, such as MPLA and MDP, can be used with the immunogenic pharmaceutical compositions disclosed herein. Monophosphoryl lipid A (MPLA), for example, is an adjuvant that causes increased presentation of liposomal antigen to specific T Lymphocytes. In addition, a muramyl dipeptide (MDP) can also be used as a suitable adjuvant in conjunction with the immunogenic pharmaceutical formulations described herein.

Adjuvant can also comprise stimulatory molecules such as cytokines. Non-limiting examples of cytokines include: CCL20, a-interferon (IFN-a), β-interferon (IFN-β), γ-interferon, platelet derived growth factor (PDGF), TNFa, TNFp, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, IL-28, MHC, CD80, CD86, IL-1, IL-2, IL-4, IL-5, IL-6, IL-10, IL-18, MCP-1, MIP-1a, MIP-1-, IL-8, L-selectin, P-selectin, E-selectin, CD34, GlyCAM-1, MadCAM-1, LFA-1, VLA-1, Mac-1, pl50.95, PECAM, ICAM-1, ICAM-2, ICAM-3, CD2, LFA-3, M-CSF, G-CSF, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-1, DR3, TRAMP, Apo-3, AIR, LARD, NGRF, DR4, DRS, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K. SAP-I, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F. TAP1, and TAP2.

Additional adjuvants include: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, IL-22, nerve growth factor, vascular endothelial growth factor, Fas, TNF receptor, Fit, Apo-l, p55, WSL-1, DR3, TRAMP, Apo-3. AIR, LARD, NGRF, DR4, DRS, KILLER, TRAIL-R2, TRICK2, DR6, Caspase ICE, Fos, c-jun, Sp-1, Ap-l, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5. TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

In some aspects, an adjuvant can be a modulator of a toll like receptor. Examples of modulators of toll-like receptors include TLR-9 agonists and are not limited to small molecule modulators of toll-like receptors such as Imiquimod. Other examples of adjuvants that are used in combination with an immunogenic pharmaceutical composition described herein can include and are not limited to saponin, CpG ODN and the like. Sometimes, an adjuvant is selected from bacteria toxoids, polyoxypropylene-polyoxyethylene block polymers, aluminum salts, liposomes, CpG polymers, oil-in-water emulsions, or a combination thereof. Sometimes, an adjuvant is an oil-in-water emulsion. The oil-in-water emulsion can include at least one oil and at least one surfactant, with the oil(s) and surfactant(s) being biodegradable (metabolisable) and biocompatible. The oil droplets in the emulsion can be less than 5 μm in diameter, and can even have a sub-micron diameter, with these small sizes being achieved with a microfluidiser to provide stable emulsions. Droplets with a size less than 220 nm can be subjected to filter sterilization.

In some instances, an immunogenic pharmaceutical composition can include carriers and excipients (including but not limited to buffers, carbohydrates, mannitol, proteins, polypeptides or amino acids such as glycine, antioxidants, bacteriostats, chelating agents, suspending agents, thickening agents and/or preservatives), water, oils including those of petroleum, animal, vegetable or synthetic origin, such as peanut oil, soybean oil, mineral oil, sesame oil and the like, saline solutions, aqueous dextrose and glycerol solutions, flavoring agents, coloring agents, detackifiers and other acceptable additives, adjuvants, or binders, other pharmaceutically acceptable auxiliary substances as required to approximate physiological conditions, such as pH buffering agents, tonicity adjusting agents, emulsifying agents, wetting agents and the like. Examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. In another instances, the pharmaceutical preparation is substantially free of preservatives. In other instances, the pharmaceutical preparation can contain at least one preservative. It will be recognized that, while any suitable carrier known to those of ordinary skill in the art can be employed to administer the pharmaceutical compositions described herein, the type of carrier will vary depending on the mode of administration.

An immunogenic pharmaceutical composition can include preservatives such as thiomersal or 2-phenoxyethanol. In some instances, the immunogenic pharmaceutical composition is substantially free from (e.g. <10 μg/ml) mercurial material e.g. thiomersal-free, α-Tocopherol succinate may be used as an alternative to mercurial compounds.

For controlling the tonicity, a physiological salt such as sodium salt can be included in the immunogenic pharmaceutical composition. Other salts can include potassium chloride, potassium dihydrogen phosphate, disodium phosphate, and/or magnesium chloride, or the like.

An immunogenic pharmaceutical composition can have an osmolality of between 200 mOsm/kg and 400 mOsm/kg, between 240-360 mOsm/kg, or within the range of 290-310 mOsm/kg.

An immunogenic pharmaceutical composition can comprise one or more buffers, such as a Tris buffer; a borate buffer; a succinate buffer; a histidine buffer (particularly with an aluminum hydroxide adjuvant): or a citrate buffer. Buffers, in some cases, are included in the 5-20 mM range.

The pH of the immunogenic pharmaceutical composition can be between about 5.0 and about 8.5, between about 6.0 and about 8.0, between about 6.5 and about 7.5, or between about 7.0 and about 7.8.

An immunogenic pharmaceutical composition can be sterile. The immunogenic pharmaceutical composition can be non-pyrogenic e.g. containing <1 EU (endotoxin unit, a standard measure) per dose, and can be <0.1 EU per dose. The composition can be gluten free.

An immunogenic pharmaceutical composition can include detergent e.g. a polyoxyethylene sorbitan ester surfactant (known as ‘Tweens’), or an octoxynol (such as octoxynol-9 (Triton X-100) or t-octylphenoxypolyethoxyethanol). The detergent can be present only at trace amounts. The immunogenic pharmaceutical composition can include less than 1 mg/mL of each of octoxynol-10 and polysorbate 80. Other residual components in trace amounts can be antibiotics (e.g. neomycin, kanamycin, polymyxin B).

An immunogenic pharmaceutical composition can be formulated as a sterile solution or suspension, in suitable vehicles, well known in the art. The pharmaceutical compositions can be sterilized by conventional, well-known sterilization techniques, or can be sterile filtered. The resulting aqueous solutions can be packaged for use as is, or lyophilized, the lyophilized preparation being combined with a sterile solution prior to administration.

An immunogenic pharmaceutical composition can be formulated with one or more pharmaceutically acceptable salts. Pharmaceutically acceptable salts can include those of the inorganic ions, such as, for example, sodium, potassium, calcium, magnesium ions, and the like. Such salts can include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, if the agent(s) contain a carboxy group or other acidic group, it can be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine, triethanolamine, and the like.

Pharmaceutical compositions comprising, for example, an active agent such as a peptide, a nucleic acid, an antibody or fragments thereof, and/or an APC described herein, in combination with one or more adjuvants can be formulated to comprise certain molar ratios. For example, molar ratios of about 99:1 to about 1:99 of an active agent such as a peptide, a nucleic acid, an antibody or fragments thereof, and/or an APC described herein, in combination with one or more adjuvants can be used. In some instances, the range of molar ratios of an active agent such as a peptide, a nucleic acid, an antibody or fragments thereof, and/or an APC described herein, in combination with one or more adjuvants can be selected from about 80:20 to about 20:80; about 75:25 to about 25:75, about 70:30 to about 30:70, about 66:33 to about 33:66, about 60:40 to about 40:60: about 50:50; and about 90:10 to about 10:90. The molar ratio of an active agent such as a peptide, a nucleic acid, an antibody or fragments thereof, and/or an APC described herein, in combination with one or more adjuvants can be about 1:9, and in some cases can be about 1:1. The active agent such as a peptide, a nucleic acid, an antibody or fragments thereof, and/or an APC described herein, in combination with one or more adjuvants can be formulated together, in the same dosage unit e.g., in one vial, suppository, tablet, capsule, an aerosol spray; or each agent, form, and/or compound can be formulated in separate units, e.g., two vials, suppositories, tablets, two capsules, a tablet and a vial, an aerosol spray, and the like.

In some instances, an immunogenic pharmaceutical composition can be administered with an additional agent. The choice of the additional agent can depend, at least in part, on the condition being treated. The additional agent can include, for example, any agents having a therapeutic effect for a pathogen infection (e.g. viral infection), including, e.g., drugs used to treat inflammatory conditions such as an NSAID, e.g., ibuprofen, naproxen, acetaminophen, ketoprofen, or aspirin. As another example, formulations can additionally contain one or more supplements, such as vitamin C, E or other anti-oxidants.

A pharmaceutical composition comprising an active agent such as a peptide, a nucleic acid, an antibody or fragments thereof, and/or an APC described herein, in combination with one or more adjuvants can be formulated in conventional manner using one or more physiologically acceptable carriers, comprising excipients, diluents, and/or auxiliaries, e.g., which facilitate processing of the active agents into preparations that can be administered. Proper formulation can depend at least in part upon the route of administration chosen. The agent(s) described herein can be delivered to a patient using a number of routes or modes of administration, including oral, buccal, topical, rectal, transdermal, transmucosal, subcutaneous, intravenous, and intramuscular applications, as well as by inhalation.

The active agents can be formulated for parenteral administration (e.g., by injection, for example bolus injection or continuous infusion) and can be presented in unit dose form in ampoules, pre-filled syringes, small volume infusion or in multi-dose containers with an added preservative. The compositions can take such forms as suspensions, solutions, or emulsions in oily or aqueous vehicles, for example solutions in aqueous polyethylene glycol.

For injectable formulations, the vehicle can be chosen from those known in art to be suitable, including aqueous solutions or oil suspensions, or emulsions, with sesame oil, corn oil, cottonseed oil, or peanut oil, as well as elixirs, mannitol, dextrose, or a sterile aqueous solution, and similar pharmaceutical vehicles. The formulation can also comprise polymer compositions which are biocompatible, biodegradable, such as poly(lactic-co-glycolic)acid. These materials can be made into micro or nanospheres, loaded with drug and further coated or derivatized to provide superior sustained release performance. Vehicles suitable for periocular or intraocular injection include, for example, suspensions of therapeutic agent in injection grade water, liposomes and vehicles suitable for lipophilic substances. Other vehicles for periocular or intraocular injection are well known in the art.

In some instances, pharmaceutical composition is formulated in accordance with routine procedures as a pharmaceutical composition adapted for intravenous administration to human beings. Typically, compositions for intravenous administration are solutions in sterile isotonic aqueous buffer. Where necessary, the composition can also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

When administration is by injection, the active agent can be formulated in aqueous solutions, specifically in physiologically compatible buffers such as Hanks solution, Ringer's solution, or physiological saline buffer. The solution can contain formulatory agents such as suspending, stabilizing and/or dispersing agents. Alternatively, the active compound can be in powder form for constitution with a suitable vehicle, e.g., sterile pyrogen-free water, before use. In another embodiment, the pharmaceutical composition does not comprise an adjuvant or any other substance added to enhance the immune response stimulated by the peptide. In another embodiment, the pharmaceutical composition comprises a substance that inhibits an immune response to the peptide.

In addition to the formulations described previously, the active agents can also be formulated as a depot preparation. Such long acting formulations can be administered by implantation or transcutaneous delivery (for example subcutaneously or intramuscularly), intramuscular injection or use of a transdermal patch. Thus, for example, the agents can be formulated with suitable polymeric or hydrophobic materials (for example as an emulsion in an acceptable oil) or ion exchange resins, or as sparingly soluble derivatives, for example, as a sparingly soluble salt.

In cases, pharmaceutical compositions comprising one or more agents exert local and regional effects when administered topically or injected at or near particular sites of infection. Direct topical application, e.g., of a viscous liquid, solution, suspension, dimethylsulfoxide (DMSO)-based solutions, liposomal formulations, gel, jelly, cream, lotion, ointment, suppository, foam, or aerosol spray, can be used for local administration, to produce for example local and/or regional effects. Pharmaceutically appropriate vehicles for such formulation include, for example, lower aliphatic alcohols, polyglycols (e.g., glycerol or polyethylene glycol), esters of fatty acids, oils, fats, silicones, and the like. Such preparations can also include preservatives (e.g., p-hydroxybenzoic acid esters) and/or antioxidants (e.g., ascorbic acid and tocopherol). See also Dermatological Formulations: Percutaneous absorption, Barry (Ed.), Marcel Dekker Incl. 1983. In another embodiment, local/topical formulations comprising a transporter, carrier, or ion channel inhibitor are used to treat epidermal or mucosal viral infections.

Pharmaceutical compositions can contain a cosmetically or dermatologically acceptable carrier. Such carriers are compatible with skin, nails, mucous membranes, tissues and/or hair, and can include any conventionally used cosmetic or dermatological carrier meeting these requirements. Such carriers can be readily selected by one of ordinary skill in the art. In formulating skin ointments, an agent or combination of agents can be formulated in an oleaginous hydrocarbon base, an anhydrous absorption base, a water-in-oil absorption base, an oil-in-water water-removable base and/or a water-soluble base. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Ointments and creams can, for example, be formulated with an aqueous or oily base with the addition of suitable thickening and/or gelling agents. Lotions can be formulated with an aqueous or oily base and will in general also containing one or more emulsifying agents, stabilizing agents, dispersing agents, suspending agents, thickening agents, or coloring agents. The construction and use of transdermal patches for the delivery of pharmaceutical agents is well known in the art. Such patches can be constructed for continuous, pulsatile, or on demand delivery of pharmaceutical agents.

Lubricants which can be used to form pharmaceutical compositions and dosage forms can include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

The pharmaceutical compositions can be in any form suitable for topical application, including aqueous, aqueous-alcoholic or oily solutions, lotion or serum dispersions, aqueous, anhydrous or oily gels, emulsions obtained by dispersion of a fatty phase in an aqueous phase (O/W or oil in water) or, conversely, (W/O or water in oil), microemulsions or alternatively microcapsules, microparticles or lipid vesicle dispersions of ionic and/or nonionic type. These compositions can be prepared according to conventional methods. The amounts of the various constituents of the compositions are those conventionally used in the art. These compositions in particular constitute protection, treatment or care creams, milks, lotions, gels or foams for the face, for the hands, for the body and/or for the mucous membranes, or for cleansing the skin. The compositions can also consist of solid preparations constituting soaps or cleansing bars.

Pharmaceutical compositions can contain adjuvants such as hydrophilic or lipophilic gelling agents, hydrophilic or lipophilic active agents, preserving agents, antioxidants, solvents, fragrances, fillers, sunscreens, odor-absorbers and dyestuffs. The amounts of these various adjuvants are those conventionally used in the fields considered and, for example, are from about 0.01% to about 20% of the total weight of the composition. Depending on their nature, these adjuvants can be introduced into the fatty phase, into the aqueous phase and/or into the lipid vesicles.

In instances relating to topical/local application, the pharmaceutical compositions can include one or more penetration enhancers. For example, the formulations can comprise suitable solid or gel phase carriers or excipients that increase penetration or help delivery of agents or combinations of agents of the invention across a permeability barrier, e.g., the skin. Many of these penetration-enhancing compounds are known in the art of topical formulation, and include, e.g., water, alcohols (e.g., terpenes like methanol, ethanol, 2-propanol), sulfoxides (e.g., dimethyl sulfoxide, decylmethyl sulfoxide, tetradecylmethyl sulfoxide), pyrrolidones (e.g., 2-pyrrolidone, N-methyl-2-pyrrolidone, N-(2-hydroxyethyl)pyrrolidone), laurocapram, acetone, dimethylacetamide, dimethylformamidc, tetrahydrofurfuryl alcohol, L-α-amino acids, anionic, cationic, amphoteric or nonionic surfactants (e.g., isopropyl myristate and sodium lauryl sulfate), fatty acids, fatty alcohols (e.g., oleic acid), amines, amides, clofibric acid amides, hexamcthylene lauramide, proteolytic enzymes, α-bisabolol, d-limonene, urea and N,N-diethyl-m-toluamide, and the like. Additional examples include humectants (e.g., urea), glycols (e.g., propylene glycol and polyethylene glycol), glycerol monolaurate, alkanes, alkanols, ORGELASE, calcium carbonate, calcium phosphate, various sugars, starches, cellulose derivatives, gelatin, and/or other polymers. In another embodiment, the pharmaceutical compositions will include one or more such penetration enhancers.

The pharmaceutical compositions for local/topical application can include one or more antimicrobial preservatives such as quaternary ammonium compounds, organic mercurials, p-hydroxy benzoates, aromatic alcohols, chlorobutanol, and the like.

The pharmaceutical compositions can be formulated into aerosol solutions, suspensions or dry powders. The aerosol can be administered through the respiratory system or nasal passages. For example, one skilled in the art will recognize that a composition of the present invention can be suspended or dissolved in an appropriate carrier, e.g., a pharmaceutically acceptable propellant, and administered directly into the lungs using a nasal spray or inhalant. For example, an aerosol formulation comprising a transporter, carrier, or ion channel inhibitor can be dissolved, suspended or emulsified in a propellant or a mixture of solvent and propellant, e.g., for administration as a nasal spray or inhalant. Aerosol formulations can contain any acceptable propellant under pressure, such as a cosmetically or dermatologically or pharmaceutically acceptable propellant, as conventionally used in the art.

An aerosol formulation for nasal administration is generally an aqueous solution designed to be administered to the nasal passages in drops or sprays. Nasal solutions can be similar to nasal secretions in that they are generally isotonic and slightly buffered to maintain a pH of about 5.5 to about 6.5, although pH values outside of this range can additionally be used. Antimicrobial agents or preservatives can also be included in the formulation.

An aerosol formulation for inhalations and inhalants can be designed so that the agent or combination of agents is carried into the respiratory tree of the subject when administered by the nasal or oral respiratory route. Inhalation solutions can be administered, for example, by a nebulizer. Inhalations or insufflations, comprising finely powdered or liquid drugs, can be delivered to the respiratory system as a pharmaceutical aerosol of a solution or suspension of the agent or combination of agents in a propellant, e.g., to aid in disbursement. Propellants can be liquefied gases, including halocarbons, for example, fluorocarbons such as fluorinated chlorinated hydrocarbons, hydrochlorofluorocarbons, and hydrochlorocarbons, as well as hydrocarbons and hydrocarbon ethers.

Halocarbon propellants can include fluorocarbon propellants in which all hydrogens are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. Hydrocarbon propellants useful in the invention include, for example, propane, isobutane, n-butane, pentane, isopentane and neopentane. A blend of hydrocarbons can also be used as a propellant.

Ether propellants include, for example, dimethyl ether as well as the ethers. An aerosol formulation of the invention can also comprise more than one propellant. For example, the aerosol formulation can comprise more than one propellant from the same class, such as two or more fluorocarbons; or more than one, more than two, more than three propellants from different classes, such as a fluorohydrocarbon and a hydrocarbon. Pharmaceutical compositions of the present invention can also be dispensed with a compressed gas, e.g., an inert gas such as carbon dioxide, nitrous oxide or nitrogen.

Aerosol formulations can also include other components, for example, ethanol, isopropanol, propylene glycol, as well as surfactants or other components such as oils and detergents. These components can serve to stabilize the formulation and/or lubricate valve components.

The aerosol formulation can be packaged under pressure and can be formulated as an aerosol using solutions, suspensions, emulsions, powders and semisolid preparations. For example, a solution aerosol formulation can comprise a solution of an agent of the invention such as a transporter, carrier, or ion channel inhibitor in (substantially) pure propellant or as a mixture of propellant and solvent. The solvent can be used to dissolve the agent and/or retard the evaporation of the propellant. Solvents can include, for example, water, ethanol and glycols. Any combination of suitable solvents can be use, optionally combined with preservatives, antioxidants, and/or other aerosol components.

An aerosol formulation can be a dispersion or suspension. A suspension aerosol formulation can comprise a suspension of an agent or combination of agents of the instant invention, e.g., a transporter, carrier, or ion channel inhibitor, and a dispersing agent. Dispersing agents can include, for example, sorbitan trioleate, oleyl alcohol, oleic acid, lecithin and corn oil. A suspension aerosol formulation can also include lubricants, preservatives, antioxidant, and/or other aerosol components.

An aerosol formulation can similarly be formulated as an emulsion. An emulsion aerosol formulation can include, for example, an alcohol such as ethanol, a surfactant, water and a propellant, as well as an agent or combination of agents of the invention, e.g., a transporter, carrier, or ion channel. The surfactant used can be nonionic, anionic or cationic. One example of an emulsion aerosol formulation comprises, for example, ethanol, surfactant, water and propellant. Another example of an emulsion aerosol formulation comprises, for example, vegetable oil, glyceryl monostearate and propane.

The pharmaceutical compounds can be formulated for administration as suppositories. A low melting wax, such as a mixture of triglycerides, fatty acid glycerides, Witepsol S55 (trademark of Dynamite Nobel Chemical, Germany), or cocoa butter is first melted and the active component is dispersed homogeneously, for example, by stirring. The molten homogeneous mixture is then poured into convenient sized molds, allowed to cool, and to solidify.

The pharmaceutical compositions can be formulated for vaginal administration. Pessaries, tampons, creams, gels, pastes, foams or sprays containing in addition to the active ingredient such carriers as are known in the art to be appropriate.

The pharmaceutical compositions can be attached releasably to biocompatible polymers for use in sustained release formulations on, in or attached to inserts for topical, intraocular, periocular, or systemic administration. The controlled release from a biocompatible polymer can be utilized with a water soluble polymer to form a instillable formulation, as well. The controlled release from a biocompatible polymer, such as for example, PLGA microspheres or nanospheres, can be utilized in a formulation suitable for intra ocular implantation or injection for sustained release administration, as well. Any suitable biodegradable and biocompatible polymer can be used.

### Production of Tissue-Specific Antigens

The present disclosure is based, at least in part, on the ability to present the immune system of the patient with one or more tissue-specific antigens. One of skill in the art from this disclosure and the knowledge in the art will appreciate that there are a variety of ways in which to produce such tissue-specific antigens. In general, such tissue-specific antigens can be produced either in vitro or in vivo. Tissue-specific antigens can be produced in vitro as peptides or polypeptides, which can then be formulated into a vaccine or pharmaceutical composition and administered to a subject. As described in further detail herein, such in vitro production can occur by a variety of methods known to one of skill in the art such as, for example, peptide synthesis or expression of a peptide/polypeptide from a DNA or RNA molecule in any of a variety of bacterial, eukaryotic, or viral recombinant expression systems, followed by purification of the expressed peptide/polypeptide. Alternatively, tissue-specific antigens can be produced in vivo by introducing molecules (e.g., DNA, RNA, viral expression systems, and the like) that encode tissue-specific antigens into a subject, whereupon the encoded tissue-specific antigens are expressed. The methods of in vitro and in vivo production of antigens are also further described herein as they relate to pharmaceutical compositions and methods of delivery of the therapy.

In Vitro Peptide Polypeptide Synthesis

Proteins or peptides of the present disclosure, e.g., tissue-specific antigenic peptides, e.g., tissue-specific antigens comprising tumor epitope sequence as provided herein, can be made by any technique known to those of skill in the art, including the expression of proteins, polypeptides or peptides through standard molecular biological techniques, the isolation of proteins or peptides from natural sources, in vitro translation, or the chemical synthesis of proteins or peptides.

Peptides of the present disclosure can be readily synthesized chemically utilizing reagents that are free of contaminating bacterial or animal substances (Merrifield RB: Solid phase peptide synthesis. I. The synthesis of a tetrapeptide. J. Am. Chem. Soc. 85:2149-54, 1%3). In some embodiments, antigenic peptides of the present disclosure are prepared by (1) parallel solid-phase synthesis on multi-channel instruments using uniform synthesis and cleavage conditions; (2) purification over a RP-HPLC column with column stripping; and re-washing, but not replacement, between peptides; followed by (3) analysis with a limited set of the most informative assays. The Good Manufacturing Practices (GMP) footprint can be defined around the set of peptides for an individual patient, thus requiring suite changeover procedures only between syntheses of peptides for different patients.

Alternatively, a nucleic acid (e.g., a polynucleotide) encoding an antigenic peptide of the present disclosure can be used to produce the antigenic peptide in vitro. The polynucleotide can be, e.g., DNA, cDNA, PNA, CNA, RNA, either single- and/or double-stranded, or native or stabilized forms of polynucleotides, such as e.g. polynucleotides with a phosphorothiate backbone, or combinations thereof and it can contain introns so long as it codes for the peptide. In one embodiment in vitro translation is used to produce the peptide. Many exemplary systems exist that one skilled in the art could utilize (e.g., Retic Lysate IVT Kit, Life Technologies, Waltham, MA). An expression vector capable of expressing a polypeptide can also be prepared. Expression vectors for different cell types are well known in the art and can be selected without undue experimentation. Generally, the DNA is inserted into an expression vector, such as a plasmid, in proper orientation and correct reading frame for expression. If necessary, the DNA can be linked to the appropriate transcriptional and translational regulatory control nucleotide sequences recognized by the desired host (e.g., bacteria), although such controls are generally available in the expression vector. The vector is then introduced into the host bacteria for cloning using standard techniques (see, e.g., Sambrook et al. (1989) Molecular Cloning, A Laboratory Manual, Cold Spring Harbor Laboratory, Cold Spring Harbor, N.Y.).

Expression vectors comprising the isolated polynucleotides, as well as host cells containing the expression vectors, are also contemplated. The antigenic peptides can be provided in the form of RNA or cDNA molecules encoding the desired antigenic peptides. One or more antigenic peptides of the disclosure can be encoded by a single expression vector.

In some embodiments, the polynucleotides can comprise the coding sequence for the tissue-specific antigenic peptide fused in the same reading frame to a polynucleotide which aids, for example, in expression and/or secretion of a polypeptide from a host cell (e.g., a leader sequence which functions as a secretory sequence for controlling transport of a polypeptide from the cell). The polypeptide having a leader sequence is a preprotein and can have the leader sequence cleaved by the host cell to form the mature form of the polypeptide.

In some embodiments, the polynucleotides can comprise the coding sequence for the antigenic peptide of the present disclosure fused in the same reading frame to a marker sequence that allows, for example, for purification of the encoded polypeptide, which can then be incorporated into a personalized vaccine or immunogenic composition. For example, the marker sequence can be a hexa-histidine tag (SEQ ID NO: 8965) supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. Additional tags include, but are not limited to, Calmodulin tags, FLAG tags, Myc tags, S tags, SBP tags, Softag 1, Softag 3, V5 tag, Xpress tag, Isopeptag, SpyTag, Biotin Carboxyl Carrier Protein (BCCP) tags, GST tags, fluorescent protein tags (e.g., green fluorescent protein tags), maltose binding protein tags, Nus tags, Strep-tag, thioredoxin tag, TC tag, Ty tag, and the like.

In some embodiments, the polynucleotides can comprise the coding sequence for one or more of the tissue-specific antigenic peptides fused in the same reading frame to create a single concatamerized antigenic peptide construct capable of producing multiple antigenic peptides.

In some embodiments, isolated nucleic acid molecules having a nucleotide sequence at least 60% identical, at least 65% identical, at least 70% identical, at least 75% identical, at least 80% identical, at least 85% identical, at least 90% identical, at least 95% identical, or at least 96%, 97%, 98% or 99% identical to a polynucleotide encoding a tissue-specific antigenic peptide of the present disclosure, can be provided.

Isolated tissue-specific antigenic peptides described herein can be produced in vitro (e.g., in the laboratory) by any suitable method known in the art. Such methods range from direct protein synthetic methods to constructing a DNA sequence encoding isolated polypeptide sequences and expressing those sequences in a suitable transformed host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g. Zoeller et al., Proc. Nat'l. Acad. Sci. USA 81:5662-5066 (1984) and U.S. Pat. No. 4,588,585.

In some embodiments, a DNA sequence encoding a polypeptide as provided herein would be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest is produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly

Once assembled (e.g., by synthesis, site-directed mutagenesis, or another method), the polynucleotide sequences encoding a particular isolated polypeptide can be inserted into an expression vector and optionally operatively linked to an expression control sequence appropriate for expression of the protein in a desired host. Proper assembly can be confirmed by nucleotide sequencing, restriction mapping, and expression of a biologically active polypeptide in a suitable host. As well known in the art, in order to obtain high expression levels of a transfected gene in a host, the gene can be operatively linked to transcriptional and translational expression control sequences that are functional in the chosen expression host.

Recombinant expression vectors can be used to amplify and express DNA encoding the tissue-specific antigenic peptides described herein. Recombinant expression vectors are replicable DNA constructs which have synthetic or cDNA-derived DNA fragments encoding a tissue-specific antigenic peptide or a bioequivalent analog operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail herein. Such regulatory elements can include an operator sequence to control transcription. The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence: or a ribosome binding site is operatively linked to a coding sequence if it is positioned so as to permit translation. Generally, operatively linked means contiguous, and in the case of secretory leaders, means contiguous and in reading frame. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

Useful expression vectors for producing polypeptides of the present disclosure in eukaryotic hosts, especially mammals or humans include, for example, vectors comprising expression control sequences from SV40, bovine papilloma virus, adenovirus and cytomegalovirus. Useful expression vectors for bacterial hosts include known bacterial plasmids, such as plasmids from Escherichia coli, including pCR 1, pBR322, pMB9 and their derivatives, wider host range plasmids, such as M13 and filamentous single-stranded DNA phages.

Suitable host cells for expression of a polypeptide of the present disclosure can include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin. Cell-free translation systems could also be employed. Appropriate cloning and expression vectors for use with bacterial, fungal, yeast, and mammalian cellular hosts are well known in the art (see Pouwels et al., Cloning Vectors: A Laboratory Manual, Elsevier, N.Y., 1985).

Various mammalian or insect cell culture systems can also be employed to express recombinant protein as provided herein. Expression of recombinant proteins in mammalian cells can be performed because such proteins are generally correctly folded, appropriately modified and completely functional. Examples of suitable mammalian host cell lines include the COS-7 lines of monkey kidney cells, described by Gluzman (Cell 23:175, 1981), and other cell lines capable of expressing an appropriate vector including, for example, L cells, C127, 3T3, Chinese hamster ovary (CHO), 293, HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, Bio/Technology 6:47 (1988).

The proteins as provided herein produced by a transformed host can be purified according to any suitable method. Such standard methods include chromatography (e.g., ion exchange, affinity and sizing column chromatography, and the like), centrifugation, differential solubility, or by any other standard technique for protein purification. Affinity tags such as hexahistidine (SEQ ID NO: 8965), maltose binding domain, influenza coat sequence, glutathione-S-transferase, and the like can be attached to the protein to allow easy purification by passage over an appropriate affinity column. Isolated proteins can also be physically characterized using such techniques as proteolysis, nuclear magnetic resonance and x-ray crystallography. For example, supernatants from systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-HPLC) steps employing hydrophobic RP-HPLC media, e.g., silica gel having pendant methyl or other aliphatic groups, can be employed to further purify a cancer stem cell protein-Fc composition. Some or all of the foregoing purification steps, in various combinations, can also be employed to provide a homogeneous recombinant protein.

Recombinant protein as described herein produced in bacterial culture can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

In Vivo Peptide/Polypeptide Synthesis

The present disclosure also contemplates the use of nucleic acid molecules as vehicles for delivering antigenic peptides/polypeptides to the subject in need thereof, in vivo, in the form of, e.g., DNA/RNA vaccines (see, e.g., WO2012/159643, and WO2012/159754, hereby incorporated by reference in their entireties).

In some embodiments, antigens can be administered to a patient in need thereof by use of a plasmid. These are plasmids which usually consist of a strong viral promoter to drive the in vivo transcription and translation of the gene (or complementary DNA) of interest (Mor, et al., (1995). The Journal of Immunology 155 (4): 2039-2046). Intron A can sometimes be included to improve mRNA stability and hence increase protein expression (Leitner, et al. (1997).The Journal of Immunology 159 (12): 6112-6119). Plasmids also include a strong polyadenylation/transcriptional termination signal, such as bovine growth hormone or rabbit beta-globulin polyadenylation sequences (Alarcon et al., (1999). Adv. Parasitol. Advances in Parasitology 42: 343-410; Robinson et al., (2000). Adv. Virus Res. Advances in Virus Research 55: 1-74; Böhmet al., (1996). Journal of Immunological Methods 193 (1): 29-40.). Multicistronic vectors are sometimes constructed to express more than one immunogen, or to express an immunogen and an immunostimulatory protein (Lewis et al., (1999). Advances in Virus Research (Academic Press) 54: 129-88).

In some embodiments, plasmids can be introduced into animal tissues by a number of different methods. Among others, two approaches can be injection of DNA in saline, using a standard hypodermic needle, and gene gun delivery. Injection in saline can be normally conducted intramuscularly (IM) in skeletal muscle, or intradermally (ID), with DNA being delivered to the extracellular spaces. This can be assisted by electroporation by temporarily damaging muscle fibers with myotoxins such as bupivacaine; or by using hypertonic solutions of saline or sucrose (Alarcon et al., (1999). Adv. Parasitol. Advances in Parasitology 42: 343-410). Immune responses to this method of delivery can be affected by many factors, including needle type, needle alignment, speed of injection, volume of injection, muscle type, and age, sex and physiological condition of the animal being injected(Alarcon et al., (1999). Adv. Parasitol. Advances in Parasitology 42: 343-410).

Gene gun delivery, also useful for the present disclosure, can ballistically accelerate plasmid DNA (pDNA) that has been adsorbed onto gold or tungsten microparticles into the target cells, using compressed helium as an accelerant (Alarcon et al., (1999). Adv. Parasitol. Advances in Parasitology 42: 343-410: Lewis et al., (1999). Advances in Virus Research (Academic Press) 54: 129-88).

Alternative delivery methods can include aerosol instillation of naked DNA on mucosal surfaces, such as the nasal and lung mucosa, (Lewis et al., (1999). Advances in Virus Research (Academic Press) 54: 129-88) and topical administration of pDNA to the eye and vaginal mucosa (Lewis et al., (1999) Advances in Virus Research (Academic Press) 54: 129-88). Mucosal surface delivery can be achieved using cationic liposome-DNA preparations, biodegradable microspheres, attenuated Shigella or Listeria vectors for oral administration to the intestinal mucosa, and recombinant adenovirus vectors. DNA or RNA can also be delivered to cells following mild mechanical disruption of the cell membrane, temporarily permeabilizing the cells. Such a mild mechanical disruption of the membrane can be accomplished by gently forcing cells through a small aperture (Ex vivo Cytosolic Delivery of Functional Macromolecules to Immune Cells, Sharei et al, PLOS ONE|DOI:10.1371/journal.pone.0118803 Apr. 13, 2015).

In some embodiments, a vaccine or pharmaceutical composition comprising tissue specific antigen can include separate DNA plasmids encoding, for example, one or more antigenic peptides/polypeptides as identified according to the disclosure. As discussed herein, the exact choice of expression vectors can depend upon the peptide/polypeptides to be expressed, and is well within the skill of the ordinary artisan. The expected persistence of the DNA constructs (e.g., in an episomal, non-replicating, non-integrated form in the muscle cells) is expected to provide an increased duration of protection.

One or more antigenic peptides of the present disclosure can be encoded and expressed in vivo using a viral based system (e.g., an adenovirus system, an adeno associated virus (AAV) vector, a poxvirus, or a lentivirus). In one embodiment, the vaccine or pharmaceutical composition can include a viral based vector for use in a human patient in need thereof, such as, for example, an adenovirus (see, e.g., Baden et al. First-in-human evaluation of the safety and immunogenicity of a recombinant adenovirus serotype 26 HIV-1 Env vaccine (IPCAVD 001). J Infect Dis. 2013 Jan. 15; 207(2):240-7, hereby incorporated by reference in its entirety). Plasmids that can be used for adeno associated virus, adenovirus, and lentivirus delivery have been described previously (see e.g., U.S. Pat. Nos. 6,955,808 and 6,943,019, and U.S. Patent application No. 20080254008, hereby incorporated by reference).

The peptides and polypeptides of the disclosure can also be expressed by a vector, e.g., a nucleic acid molecule as herein-discussed, e.g., RNA or a DNA plasmid, a viral vector such as a poxvirus, e.g., orthopox virus, avipox virus, or adenovirus, AAV or lentivirus. This approach involves the use of a vector to express nucleotide sequences that encode the peptide of the disclosure. Upon introduction into an acutely or chronically infected host or into a noninfected host, the vector can express the immunogenic peptide, and thereby can elicit a host CTL response.

Among vectors that can be used in the practice of the disclosure, integration in the host genome of a cell is possible with retrovirus gene transfer methods, often resulting in long term expression of the inserted transgene. In some embodiments, the retrovirus is a lentivirus. Additionally, high transduction efficiencies have been observed in many different cell types and target tissues. The tropism of a retrovirus can be altered by incorporating foreign envelope proteins, expanding the potential target population of target cells. A retrovirus can also be engineered to allow for conditional expression of the inserted transgene, such that only certain cell types are infected by the lentivirus. Cell type specific promoters can be used to target expression in specific cell types. Lentiviral vectors are retroviral vectors (and hence both lentiviral and retroviral vectors can be used in the practice of the disclosure). Moreover, lentiviral vectors are able to transduce or infect non-dividing cells and typically produce high viral titers. Selection of a retroviral gene transfer system can therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the desired nucleic acid into the target cell to provide permanent expression. Widely used retroviral vectors that can be used in the practice of the disclosure include those based upon murine leukemia virus (MuLV), gibbon ape leukemia virus (GaLV), Simian Immunodeficiency virus (SIV), human immunodeficiency virus (HIV), and combinations thereof (see, e.g., Buchscher et al., (1992) J. Virol. 66:2731-2739; Johann et al., (1992) J. Virol. 66:1635-1640; Sommnerfelt et al., (1990) Virol. 176:58-59; Wilson et al., (1998) J. Virol. 63:2374-2378; Miller et al., (1991) J. Virol. 65:2220-2224; PCT/US94/05700).

Also useful in the practice of the disclosure is a minimal non-primate lentiviral vector, such as a lentiviral vector based on the equine infectious anemia virus (EIAV) (see, e.g., Balagaan, (2006) J Gene Med; 8: 275-285, Published online 21 Nov. 2005 in Wiley InterScience (interscience.wiley.com). DOI: 10.1002/jgm.845). The vectors can have cytomegalovirus (CMV) promoter driving expression of the target gene. Accordingly, the disclosure contemplates amongst vector(s) useful in the practice of the disclosure: viral vectors, including retroviral vectors and lentiviral vectors.

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for delivery to the Brain, see, e.g.. US Patent Publication Nos. US20110293571; US20040013648, US20070025970, US20090111106 and U.S. Pat. No. 7,259,015. In another embodiment lentiviral vectors are used to deliver vectors to the brain of those being treated for a disease, e.g., glioma. As to lentivirus vector systems useful in the practice of the disclosure, mention is made of U.S. Pat. Nos. 6,428,953, 6,165,782, 6,013,516, 5,994,136, 6,312,682, and 7,198,784, and documents cited therein. In an embodiment herein the delivery is via an lentivirus. Zou et al, administered about 10 μL of a recombinant lentivirus having a titer of 1×10⁹ transducing units (TU)/mL by an intrathecal catheter. These sort of dosages can be adapted or extrapolated to use of a retroviral or lentiviral vector in the present disclosure. For transduction in tissues such as the brain, it is necessary to use very small volumes, so the viral preparation is concentrated by ultracentrifugation. Other methods of concentration such as ultrafiltration or binding to and elution from a matrix can be used. In other embodiments the amount of lentivirus administered can be 1×10⁵ or about 1×10⁵ plaque forming units (PFU), 5×10⁵ or about 5×10⁵ PFU, 1×10⁶ or about 1×10⁶ PFU, 5×10⁶ or about 5×10⁶ PFU, 1×10⁷ or about 1×10⁷ PFU, 5×10⁷ or about 5×10⁷ PFU, 1×10⁸ or about 1×10⁸ PFU, 5×10⁸ or about 5×10⁸ PFU, 1×10⁹ or about 1×10⁹ PFU, 5×10⁹ or about 5×10⁹PFU, 1×10¹⁰ or about 1×10¹⁰ PFU or 5×10¹⁰ or about 5×10¹⁰ PFU as total single dosage for an average human of 75 kg or adjusted for the weight and size and species of the subject. One of skill in the art can determine suitable dosage. Suitable dosages for a virus can be determined empirically.

Also useful in the practice of the disclosure is an adenovirus vector. One advantage is the ability of recombinant adenoviruses to efficiently transfer and express recombinant genes in a variety of mammalian cells and tissues in vitro and in vivo, resulting in the high expression of the transferred nucleic acids. Further, the ability to productively infect quiescent cells, expands the utility of recombinant adenoviral vectors. In addition, high expression levels ensure that the products of the nucleic acids will be expressed to sufficient levels to generate an immune response (see e.g., U.S. Pat. No. 7,029,848, hereby incorporated by reference). As to adenovirus vectors useful in the practice of the disclosure, mention is made of U.S. Pat. No. 6,955,808. The adenovirus vector used can be selected from the group consisting of the Ad5, Ad35, Ad11, C6, and C7 vectors. The sequence of the Adenovirus 5 (“Ad5”) genome has been published. (Chroboczek, J., Bieber, F., and Jacrot, B. (1992) The Sequence of the Genome of Adenovirus Type 5 and Its Comparison with the Genome of Adenovirus Type 2, Virology 186, 280-285; the contents if which is hereby incorporated by reference). Ad35 vectors are described in U.S. Pat. Nos. 6,974,695, 6,913,922, and 6,869,794. Ad11 vectors are described in U.S. Pat. No. 6,913,922. C6 adenovirus vectors are described in U.S. Pat. Nos. 6,780,407; 6,537,594; 6,309,647; 6,265,189; 6,156,567; 6,090,393; 5,942,235 and 5,833,975. C7 vectors are described in U.S. Pat. No. 6,277,558. Adenovirus vectors that are E1-defective or deleted, E3-defective or deleted, and/or E4-defective or deleted can also be used. Certain adenoviruses having mutations in the E1 region have improved safety margin because E1-defective adenovirus mutants are replication-defective in non-permissive cells, or, at the very least, are highly attenuated. Adenoviruses having mutations in the E3 region can have enhanced the immunogenicity by disrupting the mechanism whereby adenovirus down-regulates MHC class I molecules. Adenoviruses having E4 mutations can have reduced immunogenicity of the adenovirus vector because of suppression of late gene expression. Such vectors can be particularly useful when repeated re-vaccination utilizing the same vector is desired. Adenovirus vectors that are deleted or mutated in El, E3, E4, E1 and E3, and E1 and E4 can be used in accordance with the present disclosure. Furthermore, “gutless” adenovirus vectors, in which all viral genes are deleted, can also be used in accordance with the present disclosure. Such vectors require a helper virus for their replication and require a special human 293 cell line expressing both E1a and Cre, a condition that does not exist in natural environment. Such “gutless” vectors are non-immunogenic and thus the vectors can be inoculated multiple times for re-vaccination. The “gutless” adenovirus vectors can be used for insertion of heterologous inserts/genes such as the transgenes of the present disclosure, and can even be used for co-delivery of a large number of heterologous inserts/genes. In some embodiments, the delivery is via an adenovirus, which can be at a single booster dose. In some embodiments, the adenovirus is delivered via multiple doses. In terms of in vivo delivery, AAV is advantageous over other viral vectors due to low toxicity and low probability of causing insertional mutagenesis because it doesn't integrate into the host genome. AAV has a packaging limit of 4.5 or 4.75 Kb. Constructs larger than 4.5 or 4.75 Kb result in significantly reduced virus production. There are many promoters that can be used to drive nucleic acid molecule expression. AAV ITR can serve as a promoter and is advantageous for eliminating the need for an additional promoter element. For ubiquitous expression, the following promoters can be used: CMV, CAG, CBh, PGK, SV40, Ferritin heavy or light chains, etc. For brain expression, the following promoters can be used: SynapsinI for all neurons, CaMKIIalpha for excitatory neurons, GAD67 or GAD65 or VGAT for GABAergic neurons, etc. Promoters used to drive RNA synthesis can include: Pol III promoters such as U6 or H1. The use of a Pol II promoter and intronic cassettes can be used to express guide RNA (gRNA). With regard to AAV vectors useful in the practice of the disclosure, mention is made of U.S. Pat. Nos. 5,658,785, 7,115,391, 7,172,893, 6,953,690, 6,936,466, 6,924,128, 6,893,865, 6,793,926, 6,537,540, 6,475,769 and 6,258,595, and documents cited therein. As to AAV. the AAV can be AAV1, AAV2, AAV5 or any combination thereof. One can select the AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. In some embodiments the delivery is via an AAV. The dosage can be adjusted to balance the therapeutic benefit against any side effects.

In some embodiments, effectively activating a cellular immune response for a vaccine or pharmaceutical composition can be achieved by expressing the relevant antigens in a vaccine or pharmaceutical composition in a non-pathogenic microorganism. Well-known examples of such microorganisms are Mycobacterium bovis BCG. Salmonella and Pseudomonas (See, U.S. Pat. No. 6,991,797, hereby incorporated by reference in its entirety).

In some embodiments, a Poxvirus is used in the vaccine or immunogenic composition. These include orthopoxvirus, avipox, vaccinia, MVA, NYVAC, canarypox, ALVAC, fowlpox, TROVAC, etc. (see e.g., Verardi et al., Hum Vaccin Immunother. 2012 July; 8(7):961-70; and Moss, Vaccine. 2013: 31(39): 4220-4222). Poxvirus expression vectors were described in 1982 and quickly became widely used for vaccine development as well as research in numerous fields. Advantages of the vectors include simple construction, ability to accommodate large amounts of foreign DNA and high expression levels. Information concerning poxviruses that can be used in the practice of the disclosure, such as Chordopoxvirinae subfamily poxviruses (poxviruses of vertebrates), for instance, orthopoxviruses and avipoxviruses, e.g., vaccinia virus (e.g., Wyeth Strain, WR Strain (e.g., ATCC® VR-1354), Copenhagen Strain, NYVAC, NYVAC.1, NYVAC.2, MVA, MVA-BN), canarypox virus (e.g., Wheatley C93 Strain, ALVAC), fowlpox virus (e.g., FP9 Strain, Webster Strain, TROVAC), dovepox, pigeonpox, quailpox, and raccoon pox, inter alia, synthetic or non-naturally occurring recombinants thereof, uses thereof, and methods for making and using such recombinants can be found in scientific and patent literature.

In some embodiments, the vaccinia virus is used in the vaccine or pharmaceutical composition to express a tissue-specific antigen. (Rolph et al., Recombinant viruses as vaccines and immunological tools. Curr Opin Immunol 9:517-524, 1997). The recombinant vaccinia virus is able to replicate within the cytoplasm of the infected host cell and the polypeptide of interest can therefore induce an immune response. Moreover, Poxviruses have been widely used as vaccine or pharmaceutical composition vectors because of their ability to target encoded antigens for processing by the major histocompatibility complex class I pathway by directly infecting immune cells, in particular antigen-presenting cells, but also due to their ability to self-adjuvant.

In some embodiments, ALVAC is used as a vector in a vaccine or immunogenic composition. ALVAC is a canarypox virus that can be modified to express foreign transgenes and has been used as a method for vaccination against both prokaryotic and eukaryotic antigens (Hong H, Lee D S, Conkright W, et al. Phase I clinical trial of a recombinant canarypoxvirus (ALVAC) vaccine expressing human carcinoembryonic antigen and the B7.1 co-stimulatory molecule. Cancer Immunol Immunother 2000:49:504-14; von Mehren M, Arlen P, Tsang K Y, et al. Pilot study of a dual gene recombinant avipox vaccine containing both carcinoembryonic antigen (CEA) and B7.1 transgenes in patients with recurrent CEA-expressing adenocarcinomas. Clin Cancer Res 2000:6:2219-28: Musey L, Ding Y, Elizaga M, et al. HIV-1 vaccination administered intramuscularly can induce both systemic and mucosal T cell immunity in HIV-1-uninfected individuals. J Immunol 2003:171:1094-101: Paoletti E. Applications of pox virus vectors to vaccination: an update. Proc Nat Acad Sci USA 1996; 93:11349-53; U.S. Pat. No. 7,255,862). In a phase I clinical trial, an ALVAC virus expressing the tissue-specific antigen CEA showed an excellent safety profile and resulted in increased CEA-specific T-cell responses in selected patients, objective clinical responses, however, were not observed (Marshall J L, Hawkins M J, Tsang K Y, et al. Phase I study in cancer patients of a replication-defective avipox recombinant vaccine that expresses human carcinoembryonic antigen. J Clin Oncol 1999:17:332-7).

In some embodiments, a Modified Vaccinia Ankara (MVA) virus can be used as a viral vector for an antigen vaccine or immunogenic composition. MVA is a member of the Orthopoxvirus family and has been generated by about 570 serial passages on chicken embryo fibroblasts of the Ankara strain of Vaccinia virus (CVA) (for review see Mayr. A., et al., Infection 3, 6-14, 1975). As a consequence of these passages, the resulting MVA virus contains 31 kilobases less genomic information compared to CVA, and is highly host-cell restricted (Meyer. H, et al., J. Gen. Virol. 72, 1031-1038, 1991). MVA is characterized by its extreme attenuation, namely, by a diminished virulence or infectious ability, but still holds an excellent immunogenicity. When tested in a variety of animal models, MVA was proven to be avirulent, even in immuno-suppressed individuals. Moreover, MVA-BN®-HER2 is a candidate immunotherapy designed for the treatment of HER-2-positive breast cancer and is currently in clinical trials. (Mandl et al., Cancer Immunol Immunother. January 2012; 61(1): 19-29). Methods to make and use recombinant MVA has been described (e.g., see U.S. Pat. Nos. 8,309,098 and 5,185,146 hereby incorporated in its entirety).

In some embodiments, recombinant viral particles of the vaccine or pharmaceutical composition are administered to patients in need thereof.

### Modification to Peptide/Polypeptide

In some embodiments, the present disclosure includes modified antigenic peptides. A modification can include a covalent chemical modification that does not alter the primary amino acid sequence of the antigenic peptide itself. Modifications can produce peptides with desired properties, for example, prolonging the in vivo half-life, increasing the stability, reducing the clearance, altering the immunogenicity or allergenicity, enabling the raising of particular antibodies, cellular targeting, antigen uptake, antigen processing, MHC affinity, MHC stability, or antigen presentation. Changes to an antigenic peptide that can be carried out include, but are not limited to, conjugation to a carrier protein, conjugation to a ligand, conjugation to an antibody, PEGylation, polysialylation HESylation, recombinant PEG mimetics, Fc fusion, albumin fusion, nanoparticle attachment, nanoparticulate encapsulation, cholesterol fusion, iron fusion, acylation, amidation, glycosylation, side chain oxidation, phosphorylation, biotinylation, the addition of a surface active material, the addition of amino acid mimetics, or the addition of unnatural amino acids.

In some embodiments, the present disclosure also includes various modifications to overcome issues associated with short plasma half-life or susceptibility to protease degradation, including conjugating or linking the polypeptide sequence to any of a variety of non-proteinaceous polymers, e.g., polyethylene glycol (PEG), polypropylene glycol, or polyoxyalkylenes (see, for example, typically via a linking moiety covalently bound to both the protein and the nonproteinaceous polymer, e.g., a PEG). Such PEG conjugated biomolecules have been shown to possess clinically useful properties, including better physical and thermal stability, protection against susceptibility to enzymatic degradation, increased solubility, longer in vivo circulating half-life and decreased clearance, reduced immunogenicity and antigenicity, and reduced toxicity.

PEGs suitable for conjugation to a polypeptide sequence are generally soluble in water at room temperature, and have the general formula R(O—CH₂—CH₂)nO-R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure.

The present disclosure also contemplates compositions of conjugates wherein the PEGs have different n values and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods know in the art. For example, cation exchange chromatography can be used to separate conjugates, and a fraction is then identified which contains the conjugate having, for example, the desired number of PEGs attached, purified free from unmodified protein sequences and from conjugates having other numbers of PEGs attached.

PEG can be bound to a polypeptide of the present disclosure via a terminal reactive group (a “spacer”). The spacer is, for example, a terminal reactive group which mediates a bond between the free amino or carboxyl groups of one or more of the polypeptide sequences and polyethylene glycol. The PEG having the spacer which can be bound to the free amino group includes N-hydroxysuccinylimide polyethylene glycol which can be prepared by activating succinic acid ester of polyethylene glycol with N-hydroxy succinylimide. Another activated polyethylene glycol which can be bound to a free amino group is 2,4-bis(O-methoxypolyethyleneglycol)-6-chloro-s-triazine which can be prepared by reacting polyethylene glycol monomethyl ether with cyanuric chloride. The activated polyethylene glycol which is bound to the free carboxyl group includes polyoxyethylenediamine.

Conjugation of one or more of the polypeptide sequences of the present disclosure to PEG having a spacer can be carried out by various conventional methods. For example, the conjugation reaction can be carried out in solution at a pH of from 5 to 10, at temperature from 4° C. to room temperature, for 30 minutes to 20 hours, utilizing a molar ratio of reagent to protein of from 4:1 to 30:1. Reaction conditions can be selected to direct the reaction towards producing predominantly a desired degree of substitution. In general, low temperature, low pH (e.g., pH=5), and short reaction time tend to decrease the number of PEGs attached, whereas high temperature, neutral to high pH (e.g., pH>7), and longer reaction time tend to increase the number of PEGs attached. Various means known in the art can be used to terminate the reaction. In some embodiments the reaction is terminated by acidifying the reaction mixture and freezing at, e.g., −20° C.

The present disclosure also contemplates the use of PEG mimetics. Recombinant PEG mimetics have been developed that retain the attributes of PEG (e.g., enhanced serum half-life) while conferring several additional advantageous properties. By way of example, simple polypeptide chains (comprising, for example, Ala, Glu, Gly, Pro, Ser and Thr) capable of forming an extended conformation similar to PEG can be produced recombinantly already fused to the peptide or protein drug of interest (e.g., Amunix's XTEN technology: Mountain View. CA). This obviates the need for an additional conjugation step during the manufacturing process. Moreover, established molecular biology techniques enable control of the side chain composition of the polypeptide chains, allowing optimization of immunogenicity and manufacturing properties.

Glycosylation can affect the physical properties of proteins and can also be important in protein stability, secretion, and subcellular localization. The present disclosure also includes compositions comprising polypeptides with glycosylation modification. Proper glycosylation can be important for biological activity. In fact, some genes from eukaryotic organisms, when expressed in bacteria (e.g., E. coli) which lack cellular processes for glycosylating proteins, yield proteins that are recovered with little or no activity by virtue of their lack of glycosylation. Addition of glycosylation sites can be accomplished by altering the amino acid sequence. The alteration to the polypeptide can be made, for example, by the addition of, or substitution by, one or more serine or threonine residues (for O-linked glycosylation sites) or asparagine residues (for N-linked glycosylation sites). The structures of N-linked and O-linked oligosaccharides and the sugar residues found in each type can be different. One type of sugar that is commonly found on both is N-acetylneuraminic acid (hereafter referred to as sialic acid). Sialic acid is usually the terminal residue of both N-linked and O-linked oligosaccharides and, by virtue of its negative charge, may confer acidic properties to the glycoprotein. Embodiments of the present disclosure comprise the generation and use of N-glycosylation variants.

The polypeptide sequences of the present disclosure can optionally be altered through changes at the DNA level, particularly by mutating the DNA encoding the polypeptide at preselected bases such that codons are generated that will translate into the desired amino acids. Another means of increasing the number of carbohydrate moieties on the polypeptide is by chemical or enzymatic coupling of glycosides to the polypeptide. Removal of carbohydrates can be accomplished chemically or enzymatically, or by substitution of codons encoding amino acid residues that are glycosylated. Chemical deglycosylation techniques are known, and enzymatic cleavage of carbohydrate moieties on polypeptides can be achieved by the use of a variety of endo- and exo-glycosidases.

Additional suitable components and molecules for conjugation include, for example, molecules for targeting to the lymphatic system, thyroglobulin; albumins such as human serum albumin (HAS); tetanus toxoid: Diphtheria toxoid; polyamino acids such as poly(D-lysine:D-glutamic acid); VP6 polypeptides of rotaviruses; influenza virus hemagglutinin, influenza virus nucleoprotein; Keyhole Limpet Hemocyanin (KLH); and hepatitis B virus core protein and surface antigen: or any combination of the foregoing.

Fusion of albumin to one or more polypeptides of the present disclosure can, for example, be achieved by genetic manipulation, such that the DNA coding for HSA, or a fragment thereof, is joined to the DNA coding for the one or more polypeptide sequences. Thereafter, a suitable host can be transformed or transfected with the fused nucleotide sequences in the form of, for example, a suitable plasmid, so as to express a fusion polypeptide. The expression can be effected in vitro from, for example, prokaryotic or eukaryotic cells, or in vivo from, for example, a transgenic organism. In some embodiments of the present disclosure, the expression of the fusion protein is performed in mammalian cell lines, for example, CHO cell lines. Transformation is used broadly herein to refer to the genetic alteration of a cell resulting from the direct uptake, incorporation and expression of exogenous genetic material (exogenous DNA) from its surroundings and taken up through the cell membrane(s). Transformation occurs naturally in some species of bacteria, but it can also be effected by artificial means in other cells. Furthermore, albumin itself can be modified to extend its circulating half-life. Fusion of the modified albumin to one or more polypeptides can be attained by the genetic manipulation techniques described above or by chemical conjugation; the resulting fusion molecule has a half-life that exceeds that of fusions with non-modified albumin. (See WO2011/051489). Several albumin-binding strategies have been developed as alternatives for direct fusion, including albumin binding through a conjugated fatty acid chain (acylation). Because serum albumin is a transport protein for fatty acids, these natural ligands with albumin—binding activity have been used for half-life extension of small protein therapeutics. For example, insulin detemir (LEVEMIR), an approved product for diabetes, comprises a myristyl chain conjugated to a genetically-modified insulin, resulting in a long-acting insulin analog.

Another type of modification provided by the present disclosure is to conjugate (e.g., link) one or more additional components or molecules at the N- and/or C-terminus of a polypeptide sequence as provided herein, such as another protein (e.g., a protein having an amino acid sequence heterologous to the subject protein), or a carrier molecule. Thus, an exemplary polypeptide sequence can be provided as a conjugate with another component or molecule.

In some embodiments, a conjugate modification as provided herein can result in a polypeptide sequence that retains activity with an additional or complementary function or activity of the second molecule. For example, a polypeptide sequence can be conjugated to a molecule, e.g., to facilitate solubility, storage, in vivo or shelf half-life or stability, reduction in immunogenicity, delayed or controlled release in vivo, etc. Other functions or activities include a conjugate that reduces toxicity relative to an unconjugated polypeptide sequence, a conjugate that targets a type of cell or organ more efficiently than an unconjugated polypeptide sequence, or a drug to further counter the causes or effects associated with a disorder or disease as set forth herein (e.g., diabetes).

A polypeptide as provided herein can also be conjugated to large, slowly metabolized macromolecules such as proteins; polysaccharides, such as sepharose, agarose, cellulose, cellulose beads; polymeric amino acids such as polyglutamic acid, polylysine; amino acid copolymers; inactivated virus particles; inactivated bacterial toxins such as toxoid from diphtheria, tetanus, cholera, leukotoxin molecules; inactivated bacteria; and dendritic cells.

Additional candidate components and molecules for conjugation to the polypeptide sequence of the present disclosure can include those suitable for isolation or purification. Particular non-limiting examples include binding molecules, such as biotin (biotin-avidin specific binding pair), an antibody, a receptor, a ligand, a lectin, or molecules that comprise a solid support, including, for example, plastic or polystyrene beads, plates or beads, magnetic beads, test strips, and membranes. Purification methods such as cation exchange chromatography can be used to separate conjugates by charge difference, which effectively separates conjugates into their various molecular weights. The content of the fractions obtained by cation exchange chromatography can be identified by molecular weight using conventional methods, for example, mass spectroscopy, SDS-PAGE, or other known methods for separating molecular entities by molecular weight.

In some embodiments, the amino- or carboxyl-terminus of a polypeptide sequence of the present disclosure can be fused with an immunoglobulin Fc region (e.g., human Fc) to form a fusion conjugate (or fusion molecule). Fc fusion conjugates have been shown to increase the systemic half-life of biopharmaceuticals, and thus the biopharmaceutical product can require less frequent administration.

Fc can bind to the neonatal Fc receptor (FcRn) in endothelial cells that line the blood vessels, and, upon binding, the Fc fusion molecule can be protected from degradation and re-released into the circulation, keeping the molecule in circulation longer. This Fc binding can be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates.

The present disclosure also contemplates the use of other modifications, currently known or developed in the future, of the polypeptides to improve one or more properties. One such method for prolonging the circulation half-life, increasing the stability, reducing the clearance, or altering the immunogenicity or allergenicity of a polypeptide of the present disclosure can involve modification of the polypeptide sequences by hesylation, which utilizes hydroxyethyl starch derivatives linked to other molecules in order to modify the molecule's characteristics. Various aspects of hesylation are described in, for example, U.S. Patent Appln. Nos. 2007/0134197 and 2006/0258607.

In some aspects, a peptide derivative such as a tissue-specific antigen provided herein can comprise an affinity enhanced tissue-specific antigen. Such an affinity enhanced tissue-specific antigen can comprise one or more substitutions or modifications that provide enhanced immunogenicity compared to an unmodified versions of the tissue-specific antigen.

For example, an affinity enhanced tissue-specific antigen can be prepared or derived from a parent peptide, wherein affinity enhanced tissue-specific antigen contains a non-natural amino acid substituted in place of a naturally occurring amino acid residue at one or more primary anchor positions, for example at one primary anchor position, or at two primary anchor positions.

A parent peptide can be an MHCI restricted antigen and the peptide derivative can be a MHCI restricted antigen that binds at least the same MHCI molecule as the parent peptide, e.g., if the parent peptide binds HLA-A*0201, then the peptide derivative also binds HLA-A*0201. In addition, the peptide derivative may be able to trigger an expansion of T-cells that are able to bind the parent peptide when it is complexed with MHC.

The peptide derivatives may also ha % e increased immunogenicity in comparison to the parent peptide. In some embodiments, the peptide derivative exhibits at least one, or at least two, or at least three. or at least tour, or all five of the following properties.

A first property is that the peptide derivative generates a T-cell immune response that is greater than the T-cell immune response generated by the parent peptide In one embodiment, the parent peptide generates a detectable T-cell immune response, but the peptide derivative generates a T-cell immune response which is greater than the T-cell immune response generated b the parent peptide. In another embodiment, the parent peptide does not generate a detectable T-cell immune response., whereas the peptide derivative generates a T-cell immune response that can be detected. In additional embodiments, die immune response may be T-cell lysis of target cells, cytokine release, and/or T-cell degranulation.

A second property is that the peptide derivative binds to MHCI with an affinity that is higher than the affinity with which the parent peptide binds to MHCI, i.e., the peptide derivative has a lower K_{D} than the parent peptide.

A third property is that the affinity of T-cell receptors for the complex formed between MI-ICI and a peptide derivative is higher than the affinity of T-cell receptors for the complex formed between MHCI and the parent peptide. This increased affinity ma % be determined using a tetramer assay (Laugel, B., et al., 2007, J. Bicol. Chem. 282, 23799-23810; Holmberg, K., et al., 2003. J. Immunol 171.2427-2434: Yee, C., et al., 1999. J. Immunol. 162, 2227-2234).

A fourth property is that a complex formed between M1 HCl and a peptide derivative is more stable (i.e., has a slower off-rate) than a complex formed between MHCI and the parent peptide.

A fifth property is that the peptide derivative of triggers an expansion of a broader number of T-cell clones that recognize the parent peptide than are triggered by the parent peptide.

Method of Manufacturing Antigen Specific T Cells for Therapy:

Provided herein are methods for antigen specific T cell manufacturing. Provided herein are methods of preparing T cell compositions, such as therapeutic T cell compositions. For example, a method can comprise expanding or inducing antigen specific T cells. Preparing (e.g., inducing or expanding) T cells can also refer to manufacturing T cells, and broadly encompasses procedures to isolate, stimulate, culture, induce, and/or expand any type of T cells (e.g., CD4⁺ T cells and CD8⁺ T cells). In one aspect, provided herein is a method of preparing at least one antigen specific T cell comprising a T cell receptor (TCR) specific to at least one antigen peptide sequence, the method comprising incubating an APC with a population of immune cells from a biological sample depleted of cells expressing CD14 and/or CD25. In some embodiments, the method comprises preparing at least one antigen specific T cell comprising a T cell receptor (TCR) specific to at least one antigen peptide sequence, the method comprising incubating an APC with a population of immune cells from a biological sample depleted of cells expressing CD11b and/or CD19. In some embodiments, the method comprises incubating an APC with a population of immune cells from a biological sample depleted of cells expressing any CD11b and/or CD19 and/or CD14 and/or CD25 or any combination thereof.

In a second aspect, provided here is a method of preparing at least one antigen specific T cell comprising a T cell receptor (TCR) specific to at least one antigen peptide sequence, the method comprising incubating a FMS-like tyrosine kinase 3 receptor ligand (FLT3L)-stimulated APC with a population of immune cells from a biological sample.

In a third aspect, provided herein is a method of preparing a pharmaceutical composition comprising at least one antigen specific T cell comprising a T cell receptor (TCR) specific to at least one antigen peptide sequence, the method comprising: incubating FMS-like tyrosine kinase 3 receptor ligand (FLT3L) with a population of immune cells from a biological sample for a first time period; and thereafter incubating at least one T cell of the biological sample with an APC.

In a fourth aspect, provided herein is a method of preparing at least one antigen specific T cell comprising a T cell receptor (TCR) specific to at least one antigen peptide sequence, the method comprising incubating a population of immune cells from a biological sample with one or more APC preparations for one or more separate time periods of less than 28 days from incubating the population of immune cells with a first APC preparation of the one or more APC preparations, wherein at least one antigen specific memory T cell is expanded, or at least one antigen specific naïve T cell is induced.

In a fifth aspect, provided herein is a method of preparing at least one antigen specific T cell comprising a T cell receptor (TCR) specific to at least one antigen peptide sequence, the method comprising incubating a population of immune cells from a biological sample with 3 or less APC preparations for 3 or less separate time periods, wherein at least one antigen specific memory T cell is expanded or at least one antigen specific naïve T cell is induced.

In some embodiments, a method of preparing antigen specific T cells comprises a T cell receptor (TCR) specific to at least one antigen peptide sequence comprises incubating a population of immune cells from a biological sample with one or more APC preparations for one or more separate time periods, thereby stimulating T cells to become antigen specific T cells, wherein a percentage of antigen specific T cells is at least about 0.00001%, 0.00002%, 0.00005%, 0.0001%, 0.0005%,0.001%,0.005%,0.01%,0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90% or 95% of total CD4⁺ T cells, total CD8⁺ T cells, total T cells or total immune cells. In some embodiments, a method of preparing antigen specific T cells comprises a T cell receptor (TCR) specific to at least one antigen peptide sequence comprises incubating a population of immune cells from a biological sample with 3 or less APC preparations for 3 or less separate time periods, thereby stimulating T cells to become antigen specific T cells. In some embodiments, a method of preparing antigen specific T cells comprises a T cell receptor (TCR) specific to at least one antigen peptide sequence comprises incubating a population of immune cells from a biological sample with 2 or less APC preparations for 2 or less separate time periods, thereby stimulating T cells to become antigen specific T cells.

In some embodiments, provided herein is a method that comprises incubating a population of immune cells from a biological sample with one or more APC preparations for one or more separate time periods, thereby stimulating T cells to become antigen specific T cells, wherein the APC preparation is a PBMC cell population from which cells expressing one or more cell surface markers are depleted prior to antigen loading of the APC population. In some embodiments, CD14+ cells are depleted prior to antigen loading of an APC population. In some embodiments, CD25+ cells are depleted prior to antigen loading of an APC population. In some embodiments, CD11b+ cells are depleted prior to antigen loading of an APC population. In some embodiments, CD19+ cells are depleted prior to antigen loading of an APC population. In some embodiments, CD3+ cells are depleted prior to antigen loading of an APC population. In some embodiments, CD25+ cells and CD14+ cells are depleted prior to antigen loading of an APC population.

In some embodiments, CD11b+ and CD25+ cells are depleted prior to antigen loading of an APC population. In some embodiments, CD11b+ and CD14+ cells are depleted prior to antigen loading of an APC population. In some embodiments, CD11b+, CD14+ and CD25+ cells are depleted prior to antigen loading of an APC population. In some embodiments, CD11b+, and CD19+ cells are depleted prior to antigen loading of an APC population. In some embodiments, CD11b+, CD19+ and CD25+ cells are depleted prior to antigen loading of an APC population. In some embodiments, CD11b+, CD14+, CD19+ and CD25+ cells are depleted prior to antigen loading of an APC population. In some embodiments, the method comprises adding to any of the depleted APC population described above, an APC enriched cell PBMC-derived population that are depleted of CD3+ cell. In some embodiments, the APC enriched cell PBMC-derived population is depleted of CD3+ and cells depleted of any one or more of CD11b+, CD14+, CD19+, or CD25+.

In some embodiments, a biological sample comprises peripheral blood mononuclear cells (PBMCs). In some embodiments, the method comprises adding to a PBMC sample, a composition comprising one or more antigenic peptides or nucleic acids encoding the same, thereby loading the APCs within the PBMCs with antigens for antigen presentation to T cells in the PBMC.

In some embodiments, a method comprises: (a) obtaining a biological sample from a subject comprising at least one antigen presenting cell (APC): (b) enriching cells expressing CD11c from the biological sample, thereby obtaining a CD11c⁺ cell enriched sample; (c) incubating the CD11c⁺ cell enriched sample with at least one cytokine or growth factor for a first time period; (d) incubating at least one peptide with the CD11c⁺ enriched sample of (c) for a second time period, thereby obtaining an APC peptide loaded sample; (e) incubating the APC peptide loaded sample with one or more cytokines or growth factors for a third time period, thereby obtaining a matured APC sample; (f) incubating APCs of the matured APC sample with a CD11b and/or CD14 and/or CD25 depleted sample comprising PBMCs for a fourth time period; (g) incubating the PBMCs with APCs of a matured APC sample for a fifth time period; (h) incubating the PBMCs with APCs of a matured APC sample for a sixth time period; and (i) administering at least one T cell of the PBMCs to a subject in need thereof.

In some embodiments, a method comprises: (a) obtaining a biological sample from a subject comprising at least one antigen presenting cell (APC); (b) enriching cells expressing CD14 from the biological sample, thereby obtaining a CD14⁺ cell enriched sample; (c) incubating the CD14⁺ cell enriched sample with at least one cytokine or growth factor for a first time period; (d) incubating at least one peptide with the CD14⁺ enriched sample of (c) for a second time period, thereby obtaining an APC peptide loaded sample: (e) incubating the APC peptide loaded sample with one or more cytokines or growth factors for a third time period, thereby obtaining a matured APC sample; (f) incubating APCs of the matured APC sample with a CD14 and/or CD25 depleted sample comprising PBMCs for a fourth time period; (g) incubating the PBMCs with APCs of a matured APC sample for a fifth time period; (h) incubating the PBMCs with APCs of a matured APC sample for a sixth time period; and (i) administering at least one T cell of the PBMCs to a subject in need thereof.

In some embodiments, a method comprises: (a) obtaining a biological sample from a subject comprising at least one APC and at least one PBMC; (b) depleting cells expressing CD11b and/or CD19 from the biological sample, thereby obtaining a CD11b and/or CD19 cell depleted sample; (c) incubating the CD11b and/or CD19 cell depleted sample with FLT3L for a first time period; (d) incubating at least one peptide with the CD11b and/or CD19 cell depleted sample of (c) for a second time period, thereby obtaining an APC peptide loaded sample: (e) incubating the APC peptide loaded sample with the at least one PBMC for a third time period, thereby obtaining a first stimulated PBMC sample; (f) incubating a PBMC of the first stimulated PBMC sample with an APC of a matured APC sample for a fourth time period, thereby obtaining a second stimulated PBMC sample: (g) incubating a PBMC of the second stimulated PBMC sample with an APC of a matured APC sample for a fifth time period, thereby obtaining a third stimulated PBMC sample; (h) administering at least one T cell of the third stimulated PBMC sample to a subject in need thereof.

In some embodiments, a method comprises: (a) obtaining a biological sample from a subject comprising at least one APC and at least one PBMC; (b) depleting cells expressing CD11b and/or CD19 and/or CD14 and/or CD25 from the biological sample, thereby obtaining a CD11b and/or CD19 cell depleted sample; (c) incubating the CD11b and/or CD19 and/or CD14 and/or CD25 cell depleted sample with FLT3L for a first time period; (d) incubating at least one peptide with the CD11b and/or CD19 and/or CD14 and/or CD25 cell depleted sample of (c) for a second time period, thereby obtaining an APC peptide loaded sample: (e) incubating the APC peptide loaded sample with the at least one PBMC for a third time period, thereby obtaining a first stimulated PBMC sample; (f) incubating a PBMC of the first stimulated PBMC sample with an APC of a matured APC sample for a fourth time period, thereby obtaining a second stimulated PBMC sample; (g) incubating a PBMC of the second stimulated PBMC sample with an APC of a matured APC sample for a fifth time period, thereby obtaining a third stimulated PBMC sample; (h) administering at least one T cell of the third stimulated PBMC sample to a subject in need thereof.

In some embodiments, a method comprises: (a) obtaining a biological sample from a subject comprising at least one APC and at least one PBMC; (b) depleting cells expressing CD14 and/or CD25 from the biological sample, thereby obtaining a CD14 and/or CD25 cell depleted sample; (c) incubating the CD14 and/or CD25 cell depleted sample with FLT3L for a first time period; (d) incubating at least one peptide with the CD14 and/or CD25 cell depleted sample of (c) for a second time period, thereby obtaining an APC peptide loaded sample; (e) incubating the APC peptide loaded sample with the at least one PBMC for a third time period, thereby obtaining a first stimulated PBMC sample; (f) incubating a PBMC of the first stimulated PBMC sample with an APC of a matured APC sample for a fourth time period, thereby obtaining a second stimulated PBMC sample; (g) incubating a PBMC of the second stimulated PBMC sample with an APC of a matured APC sample for a fifth time period, thereby obtaining a third stimulated PBMC sample: (h) administering at least one T cell of the third stimulated PBMC sample to a subject in need thereof.

In some embodiments, a method of preparing at least one antigen specific T cell comprising a T cell receptor (TCR) specific to at least one antigen peptide sequence comprises incubating an APC with a population of immune cells from a biological sample depleted of cells expressing CD14 and/or CD25.

In some embodiments, provided herein is a method of preparing at least one antigen specific T cell comprising a T cell receptor (TCR) specific to at least one antigen peptide sequence, the method comprising incubating a population of immune cells from a biological sample with one or more APC preparations for one or more separate time periods of less than 28 days from incubating the population of immune cells with a first APC preparation of the one or more APC preparations, wherein at least one antigen specific memory T cell is expanded, or at least one antigen specific naïve T cell is induced. In some embodiments, provided herein is a method of preparing at least one antigen specific T cell comprising a T cell receptor (TCR) specific to at least one antigen peptide sequence, the method comprising incubating a population of immune cells from a biological sample with 3 or less APC preparations for 3 or less separate time periods, wherein at least one antigen specific memory T cell is expanded or at least one antigen specific naïve T cell is induced.

In some embodiments, a method of preparing antigen specific T cells comprises a T cell receptor (TCR) specific to at least one antigen peptide sequence comprises contacting a population of immune cells (e.g., PBMCs) to APCs. In some embodiments, a method of preparing antigen specific T cells comprises a T cell receptor (TCR) specific to at least one antigen peptide sequence comprises incubating a population of immune cells (e.g., PBMCs) with APCs for a time period. In some embodiments, the population of immune cells is from a biological sample. In some embodiments, the population of immune cells is from a sample (e.g., a biological sample) depleted of CD14 expressing cells. In some embodiments, the population of immune cells is from a sample (e.g., a biological sample) depleted of CD25 expressing cells. In some embodiments, the population of immune cells is from a sample (e.g., a biological sample) depleted of CD14 expressing cells and CD25 expressing cells.

In some embodiments, a method of preparing at least one antigen specific T cell comprising a T cell receptor (TCR) specific to at least one antigen peptide sequence comprises incubating a FMS-like tyrosine kinase 3 receptor ligand (FLT3L)-stimulated APC with a population of immune cells from a biological sample. In some embodiments, provided herein is a method of preparing a pharmaceutical composition comprising at least one antigen specific T cell comprising a T cell receptor (TCR) specific to at least one antigen peptide sequence, the method comprising: incubating FMS-like tyrosine kinase 3 receptor ligand (FLT3L) with a population of immune cells from a biological sample for a first time period: and thereafter incubating at least one T cell of the biological sample with an APC.

In some embodiments, a method of preparing at least one antigen specific T cell comprising a T cell receptor (TCR) specific to at least one antigen peptide sequence comprises contacting a population of immune cells from a sample (e.g., a biological sample) with FMS-like tyrosine kinase 3 receptor ligand (FLT3L). In some embodiments, a method of preparing at least one antigen specific T cells comprises a T cell receptor (TCR) specific to at least one antigen peptide sequence comprises contacting a population of immune cells from a sample (e.g., a biological sample) with FMS-like tyrosine kinase 3 receptor ligand (FLT3L)-stimulated APCs. In some embodiments, a method of preparing at least one antigen specific T cells comprises a T cell receptor (TCR) specific to at least one antigen peptide sequence comprises incubating a population of immune cells from a sample (e.g., a biological sample) with FMS-like tyrosine kinase 3 receptor ligand (FLT3L)-stimulated APCs. In some embodiments, a method of preparing a pharmaceutical composition comprising at least one antigen specific T cell comprising a T cell receptor (TCR) specific to at least one antigen peptide sequence comprises incubating FMS-like tyrosine kinase 3 receptor ligand (FLT3L) with a population of immune cells from a biological sample (e.g., for a time period): and then contacting T cells of the biological sample to APCs. In some embodiments, a method of preparing at least one antigen specific T cell comprising a T cell receptor (TCR) specific to at least one antigen peptide sequence comprises contacting a population of immune cells from a sample (e.g., a biological sample) to one or more APC preparations. In some embodiments, a method of preparing at least one antigen specific T cell comprising a T cell receptor (TCR) specific to at least one antigen peptide sequence comprises incubating a population of immune cells from a sample (e.g., a biological sample) to one or more APC preparations for one or more separate time periods. In some embodiments, a method of preparing at least one antigen specific T cell comprising a T cell receptor (TCR) specific to at least one antigen peptide sequence comprises incubating a population of immune cells from a sample (e.g., a biological sample) to one or more APC preparations for 1, 2, 3, 4, 5, 6, 7, 8, 9 or 10 separate time periods. In some embodiments, the one or more separate time periods is less than 28 days calculated from incubating the population of immune cells with a first APC preparation of the one or more APC preparations.

In some embodiments, a method of preparing antigen specific T cells comprises a T cell receptor (TCR) specific to at least one antigen peptide sequence comprises incubating a population of immune cells to APCs for a time period, wherein the population of immune cells is from a biological sample comprising PBMCs. In some embodiments, a method of preparing antigen specific T cells comprises a T cell receptor (TCR) specific to at least one antigen peptide sequence comprises incubating a population of immune cells to APCs for a time period, wherein the population of immune cells is from a biological sample depleted of CD14 and/or CD25 expressing cells.

In some embodiments, a method of preparing antigen specific T cells comprising a T cell receptor (TCR) specific to at least one antigen peptide sequence comprises incubating a population of immune cells from a biological sample with FMS-like tyrosine kinase 3 receptor ligand (FLT3L)-stimulated APCs for a time period.

In some embodiments, a method of preparing a pharmaceutical composition comprising antigen specific T cells comprising a T cell receptor (TCR) specific to at least one antigen peptide sequence comprises incubating FMS-like tyrosine kinase 3 receptor ligand (FLT3L) with a population of immune cells from a biological sample; and then contacting T cells of the biological sample with APCs.

In some embodiments, a method of preparing antigen specific T cells comprising a T cell receptor (TCR) specific to at least one antigen peptide sequence comprises incubating a population of immune cells from a biological sample with one or more APC preparations for one or more separate time periods, thereby inducing or expanding antigen specific T cells, wherein the one or more separate time periods is less than 28 days calculated from incubating the population of immune cells with a first APC preparation of the one or more APC preparations. In some embodiments, incubating a population of immune cells from a biological sample with one or more APC preparations for one or more separate time periods is performed in a medium containing IL-7, IL-15, or a combination thereof. In some embodiments, the medium further comprises an indoleamine 2,3-dioxygenase-I (IDO) inhibitor, an anti-PD-1 antibody, IL-12, or a combination thereof. The IDO inhibitor can be epacadostat, navoximod, 1-Methyltryptophan, or a combination thereof. In some embodiments, the IDO inhibitor may increase the number of antigen-specific CD8⁺ cells. In some embodiments, the IDO inhibitor may maintain the functional profile of memory CD8⁺ T cell responses. The PD-1 antibody may increase the absolute number of antigen-specific memory CD8+ T cell responses. The PD-1 antibody may increase proliferation rate of the cells treated with such antibody. The additional of IL-12 can result in an increase of antigen-specific cells and/or an increase in the frequency of CD8⁺ T cells.

In some embodiments, a method of preparing antigen specific T cells comprising a T cell receptor (TCR) specific to at least one antigen peptide sequence comprises incubating a population of immune cells comprising from a biological sample with one or more APC preparations for one or more separate time periods, thereby expanding or inducing antigen specific T cells, wherein a percentage of antigen specific T cells, antigen specific CD4⁺ T cells, or antigen specific CD8⁺ T cells is at least about 0.00001%, 0.00002%, 0.00005%, 0.0001%, 0.0005%, 0.001%, 0.005%, 0.01%, 0.05%, 0.1%, 0.5%, 1%, 2%, 3%, 4%, 5%, 6%, 7%,8%,9/, 10%., 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90% or 95% of total T cells, total CD4⁺ T cells, total CD8⁺ T cells, total immune cells, or total cells.

In some embodiments, a method of preparing antigen specific T cells comprises a T cell receptor (TCR) specific to at least one antigen peptide sequence comprises incubating a population of immune cells from a biological sample with 3 or less APC preparations for 3 or less separate time periods, thereby stimulating T cells to become antigen specific T cells.

In some embodiments, the population of immune cells is from a biological sample depleted of CD14 and/or CD25 expressing cells. In some embodiments, the APCs are FMS-like tyrosine kinase 3 receptor ligand (FLT3L)-stimulated APCs. In some embodiments, the APCs comprise one or more APC preparations. In some embodiments, the APC preparations comprise 3 or less APC preparations. In some embodiments, the APC preparations are incubated with the immune cells sequentially within one or more separate time periods.

In some embodiments, the biological sample is from a subject. In some embodiments, the subject is a human. For example, the subject can be a patient or a donor. In some embodiments, the subject has a disease or disorder. In some embodiments, the disease or disorder is cancer. In some embodiments, the antigen specific T cells comprise CD4⁺ and/or CD8⁺ T cells. In some embodiments, the antigen specific T cells comprise CD4 enriched T cells and/or CD8 enriched T cells. For example, a CD4⁺ T cell and/or CD8⁺ T cell can be isolated from, enriched from, or purified from a biological sample from a subject comprising PBMCs. In some embodiments, the antigen specific T cells are naïve CD4⁺ and/or naïve CD8⁺ T cells. In some embodiments, the antigen specific T cells are memory CD4⁺ and/or memory CD8⁺ T cells.

In some embodiments, the at least one antigen peptide sequence comprises a mutation selected from (A) a point mutation and the cancer antigen peptide binds to the HLA protein of the subject with an IC₅₀ less than 500 nM and a greater affinity than a corresponding wild-type peptide, (B) a splice-site mutation, (C) a frameshift mutation, (D) a read-through mutation, (E) a gene-fusion mutation, and combinations thereof. In some embodiments, each of the at least one antigen peptide sequence binds to a protein encoded by an HLA allele expressed by the subject. In some embodiments, each of the at least one antigen peptide sequence comprises a mutation that is not present in non-cancer cells of the subject. In some embodiments, each of the at least one antigen peptide sequences is encoded by an expressed gene of the subject's cancer cells. In some embodiments, one or more of the at least one antigen peptide sequence has a length of from 8-50 naturally occurring amino acids. In some embodiments, the at least one antigen peptide sequence comprises a plurality of antigen peptide sequences. In some embodiments, the plurality of antigen peptide sequences comprises from 2-50, 3-50, 4-50, 5-5-, 6-50, 7-50, 8-50, 9-50, or 10-50 antigen peptide sequences.

In some embodiments, the APCs comprise APCs loaded with one or more antigen peptides comprising one or more of the at least one antigen peptide sequence. In some embodiments, the APCs are autologous APCs or allogenic APCs. In some embodiments, the APCs comprise dendritic cells (DCs).

In some embodiments, a method comprises depleting CD14 and/or CD25 expressing cells from the biological sample. In some embodiments, depleting CD14⁺ cells comprises contacting a CD14 binding agent to the APCs. In some embodiments, the APCs are derived from CD14⁺ monocytes. In some embodiments, the APCs are enriched from the biological sample. For example, an APC can be isolated from, enriched from, or purified from a biological sample from a subject comprising PBMCs.

In some embodiments, the APCs are stimulated with one or more cytokines or growth factors. In some embodiments, the one or more cytokines or growth factors comprise GM-CSF, IL-4, FLT3L, or a combination thereof. In some embodiments, the one or more cytokines or growth factors comprise IL-4, IFN-γ, LPS, GM-CSF, TNF-α, IL-1β, PGE1, IL-6, IL-7 or a combination thereof.

In some embodiments, the APCs are from a second biological sample. In some embodiments, the second biological sample is from the same subject.

In some embodiments, a percentage of antigen specific T cells in the method is at least about 1%, 2%, 3%, 4%, 5%, 6%, 7%, 8%, 9%, 10%, 11%, 12%, 13%, 14%, 15%, 16%, 17%, 18%, 19%, or 20% of total T cells or total immune cells. In some embodiments, a percentage of antigen specific T cells in the method is from about 0.1% to about 5%, from about 5% to 10%, from about 10% to 15%, from about 15% to 20%, from about 20% to 25%, from about 25% to 30%, from about 30% to 35%, from about 35% to about 40%, from about 40% to about 45%, from about 45% to about 50%, from about 50% to about 55%, from about 55% to about 60%, from about 60% to 65%, or from about 65% to about 70% of total T cells or total immune cells. In some embodiments, a percentage of antigen specific CD8⁺ T cells in the method is at least about 1%, 2%, 3%, 4%, 5%, 6%, 7%, 8%, 9%, 10%, 11%, 12%, 13%, 14%, 15%, 16%, 17%, 18%, 19%, or 20% of total T cells or total immune cells. In some embodiments, a percentage of antigen specific naïve CD8⁺ T cells in the method is at least about 1%, 2%, 3%, 4%, 5%, 6%, 7%, 8%, 9%, 10%, 11%, 12%, 13%, 14%, 15%, 16%, 17%, 18%, 19%, or 20% of total T cells or total immune cells. In some embodiments, a percentage of antigen specific memory CD8 T cells in the method is at least about 1%, 2%, 3%, 4%, 5%, 6%, 7%, 8%, 9%, 10%, 11%, 12%, 13%, 14%, 15%, 16%, 17%, 18%, 19%, or 20% of total T cells or total immune cells. In some embodiments, a percentage of antigen specific CD4⁺ T cells in the method is at least about 1%, 2%, 3%, 4%, 5%, 6%, 7%, 8%, 9%, 10%, 11%, 12%, 13%, 14%, 15%, 16%, 17%, 18%, 19%, or 20% of total T cells or total immune cells. In some embodiments, a percentage of antigen specific CD4⁺ T cells in the method is at least about 1%, 2%, 3%, 4%, 5%, 6%, 7%, 8%, 9%, 10%, 11%, 12%, 13%, 14%, 15%, 16%, 17%, 18%, 19%, or 20% of total T cells or total immune cells. In some embodiments, a percentage of antigen specific T cells in the biological sample is at most about 0.5%, 1%, 2%, 3%, 4%, 5%, 6%, 7%, 8%, 9%, 10%, 11%, 12%, 13%, 14%, 15%, 16%, 17%, 18%, 19%, or 20%. In some embodiments, a percentage of antigen specific CD8⁺ T cells in the biological sample is at most about 0.5%, 1%, 2%, 3%, 4%, 5%, 6%, 7%, 8%, 9%, 10%, 11%, 12%, 13%, 14%, 15%, 16%, 17%, 18%, 19%, or 20%. In some embodiments, a percentage of antigen specific naïve CD8⁺ T cells in the biological sample is at most about 0.5%, 1%, 2%, 3%, 4%, 5%, 6%, 7%, 8%, 9%, 10%, 11%, 12%, 13%, 14%, 15%, 16%, 17%, 18%, 19%, or 20%. In some embodiments, a percentage of antigen specific memory CD8⁺ T cells in the biological sample is at most about 0.5%, 1%, 2%, 3%, 4%, 5%, 6%, 7%, 8%, 9%, 10%, 11%, 12%, 13%, 14%, 15%, 16%, 17%, 18%, 19%, or 20%. In some embodiments, a percentage of antigen specific CD4⁺ T cells in the biological sample is at most about 0.5%, 1%, 2%, 3%, 4%, 5%, 6%, 7%, 8%, 9%, 10%, 11%, 12%, 13%, 14%, 15%, 16%, 17%, 18%, 19%, or 20%.

In some embodiments, a biological sample is freshly obtained from a subject or is a frozen sample.

In some embodiments, a method comprises incubating one or more of the APC preparations with a first medium comprising at least one cytokine or growth factor for a first time period. In some embodiments, the first time period is at lease 1, 2, 3, 4, 5, 6, 7, 8, 9, 10, 11, 12, 13, 14, 15, 16, or 17, or 18 days. In some embodiments, the first time period is no more than 2, 3, 4, 5, 6, 7, 8, 9, 10, 11, 12, 13, 14, 15, 16, 17, or 18 days. In some embodiments, the first time period is at least 1, 2 3, 4, 5, 6, 7, 8, or 9 days.

In some embodiments, the first time period is no more than 3, 4, 5, 6, 7, 8, 9, or 10 days. In some embodiments, the at least one cytokine or growth factor comprises GM-CSF, IL-4, FLT3L, TNF-α, IL-1β, PGE1, IL-6, IL-7, IFN-γ, LPS, IFN-α, R848, LPS, ss-ma40, poly I:C, or any combination thereof.

In some embodiments, a method comprises incubating one or more of the APC preparations with at least one peptide for a second time period. In some embodiments, the second time period is no more than 1 hour.

In some embodiments, a method comprises incubating one or more of the APC preparations with a second medium comprising one or more cytokines or growth factors for a third time period, thereby obtaining matured APCs. In some embodiments, the one or more cytokines or growth factors comprises GM-CSF (granulocyte macrophage colony-stimulating factor), IL-4, FLT3L, IFN-γ, LPS, TNF-α, IL-1β, PGE1, IL-6, IL-7, IFN-α, R848 (resiquimod), LPS, ss-rna40, poly I:C, CpG, or a combination thereof. In some embodiments, the third time period is no more than 2, 3, 4, 5, 6, 7, 8, 9, 10, 11, 12, 13, 14, 15, 16, 17, or 18 days. In some embodiments, the third time period is at least 1, 2, 3, 4, 5, 6, 7, 8, 9, 10, 11, 12, 13, 14, 15, 16, or 17 days. In some embodiments, the third time period is no more than 2, 3, 4, or 5 days. In some embodiments, the third time period is at least 1, 2, 3, or 4 days.

In some embodiment, the method further comprises removing the one or more cytokines or growth factors of the second medium after the third time period and before a start of the fourth time period.

Antigen Loaded PBMCs for T Cell Induction In Vitro

In some embodiments, the methods provided herein comprise isolating PBMCs from a human blood sample, and directly loading the PBMCs with antigens. PBMCs directly contacted with antigens can readily take up antigens by phagocytosis and present antigens to T cells that may be in the culture or added to the culture. In some embodiments, the methods provided herein comprise isolating PBMCs from a human blood sample, and nucleofecting or electroporating a polynucleotide, such as an mRNA, that encodes one or more antigens into the PBMCs. In some embodiments, antigens delivered to PBMCs, instead of antigen presenting cells maturing to DCs, provides a great advantage in terms of time and manufacturing efficiency. The PBMCs may be further depleted of one or more cell types. In some embodiments, the PBMCs may be depleted of CD3+ cells for an initial period of antigen loading and the CD3+ cells returned to the culture for the PBMCs to stimulate the CD3+ T cells. In some embodiments, the PBMCs may be depleted of CD25+ cells. In some embodiments, the PBMCs may be depleted of CD14+ cells. In some embodiments, the PBMCs may be depleted of CD19+ cells. In some embodiments, the PBMCs may be depleted of both CD14 and CD25 expressing cells. In some embodiments, CD11b+ cells are depleted from the PBMC sample before antigen loading. In some embodiments, CD11b+ and CD25+ cells are depleted from the PBMC sample before antigen loading.

In some embodiments, the PBMCs isolated from a human blood sample may be handled as minimally as possible prior to loading with antigens. Increased handling of PBMCs, for example freezing and thawing cells, multiple cell depletion steps, etc., may impair cell health and viability.

In some embodiments, the PBMCs are allogeneic to the subject of therapy. In some embodiments the PBMCs are allogeneic to the subject of adoptive cell therapy with antigen specific T cells.

In some embodiments, the PBMCs are HLA-matched for the subject of therapy. In some embodiments, the PBMCs are allogeneic, and matched for the subject's HLA subtypes, whereas the CD3+ T cells are autologous. The PBMCs are loaded with the respective antigens (e.g. derived from analysis of a peptide presentation analysis platform such as RECON), cocultured with subject's PBMC comprising T cells in order to stimulate antigen specific T cells.

In some embodiments, mRNA is used as the immunogen for uptake and antigen presenting. One advantage of using mRNA over peptide antigens to load PBMCs is that RNA is self adjuvanting, and does not require additional adjuvants. Another advantage of using mRNA is that the peptides are processed and presented endogenously. In some embodiments, the mRNA comprises shortmer constructs, encoding 9-10 amino acid peptides comprising an epitope. In some embodiments, the mRNA comprises longmer constructs, encoding bout 25 amino acid peptides. In some embodiments, the mRNA comprises a concatenation of multiple epitopes. In some embodiments, the concatemers may comprise one or more epitopes from the same antigenic protein. In some embodiments, the concatemers may comprise one or epitopes from several different antigenic proteins. Several embodiments are described in the Examples section. Antigen loading of PBMCs by antigen loading may comprise various mechanisms of delivery ad incorporation of nucleic acid into the PBMCs. In some embodiments, the delivery or mechanism of incorporation includes transfection, electroporation, nucleofection, chemical delivery, for example, lipid encapsulated or liposome mediated delivery.

Use of antigen loaded PBMCs to stimulate T cells saves the maturation time required in a method that generates DCs from a PBMC sample prior to T cell stimulation. In some embodiments, use of antigen loaded PBMCs, for example, mRNA loaded PBMCs as APCs reduces the total manufacturing time by 1, 2, 3, 4, 5, 6, 7, 8, 9, or 10 days. In some embodiments, use of antigen loaded PBMCs as APCs reduces the total manufacturing time by 3 days. In some embodiments, use of antigen loaded PBMCs as APCs reduces the total manufacturing time by 4 days. In some embodiments, use of antigen loaded PBMCs as APCs reduces the total manufacturing time by 5 days. In some embodiments, use of antigen loaded PBMCs as APCs reduces the total manufacturing time by 6 days. In some embodiments, use of antigen loaded PBMCs as APCs reduces the total manufacturing time by 7 days.

In some embodiments, use of mRNA as antigen may be preferred because it is easy to design and manufacture nucleic acids, and transfect the PBMCs. In some embodiments, mRNA loaded PBMCs can stimulate T cells and generate higher antigen specific T cells. In some embodiments, mRNA loaded PBMCs can stimulate T cells and generate higher yield of antigen specific T cells. In some embodiments, mRNA loaded PBMCs can stimulate T cells and generate antigen specific T cells that have higher representation of the input antigens, i.e., reactive to diverse antigens. In some embodiments, mRNA loaded PBMCs can stimulate T cells that have at least 1, 2, 3, 4, 5, 6, 7, 8, 9, 10 or more antigen reactivity in the pool of expanded cells. In some embodiments, the mRNA loaded PBMCs can stimulate T cells that have at least 1, 2, 3, 4, 5, 6, 7, 8, 9, 10 or more antigen reactivity than conventional antigen loaded APCs (such as peptide loaded DCs).

### EXAMPLES

The examples provided below are for illustrative purposes only and do not to limit the scope of the claims provided herein.

### Example 1. Identification of Tissue-Specific Gene Expression and Tissue-Specific Antigen

Examples 1 and 2 exemplify-the methods of identification of tissue-specific antigen or epitope sequence according to some embodiments of the present disclosure. Here, systematic efforts were taken to discover tissue-specific antigens capable of eliciting a TCR-mediated response.

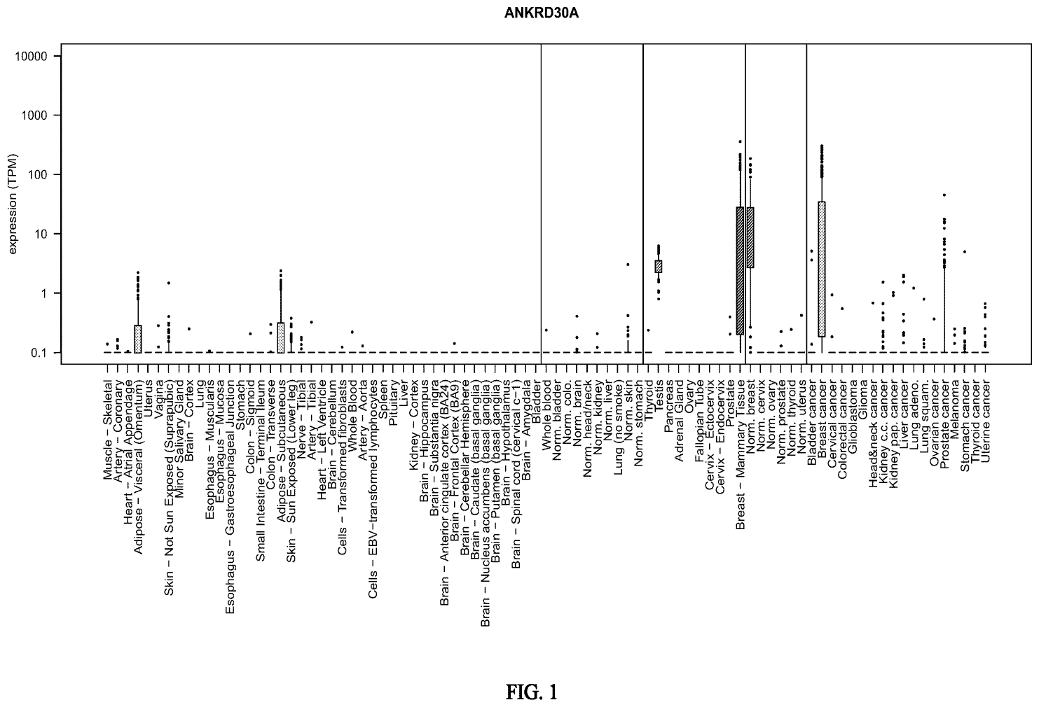

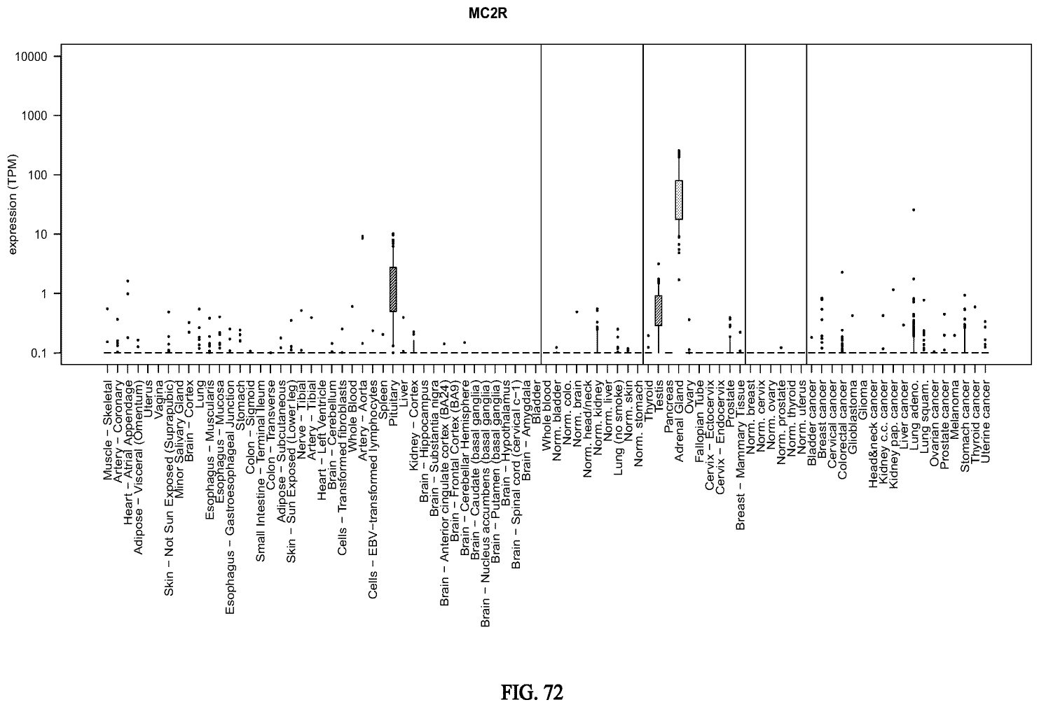

As a first step, gene expression in cancer and non-cancer tissue types profiled in the TCGA and GTEX data sets was screened through by a bioinformatic program. Each tissue type was categorized as essential or non-essential. All tumor tissues were considered non-essential whereas normal tissues could be considered essential (e.g. brain, colon, etc.) or non-essential (e.g. ovary, prostate, thyroid, etc). This process uncovered a small set of genes whose expression profile was restricted in the desired way. FIGS. 1-72 are boxplots illustrating expression levels of these genes ANKRD30A, COL10A1, CTCFL, PPIAL4G, POTEE, DLL3, MMP13, SSX1, DCAF4L2, MAGEA4, MAGEA11, MAGEC2, MAGEA12, PRAME, CLDN6, EPYC, KLK3, KLK2, KLK4, TGM4, POTEG, RLN1, POTEH, SLC45A2, TSPAN10, PAGE5, CSAG1, PRDM7, TG, TSHR, RSPH6A, SCXB, HIST1H4K, ALPPL2, PRM2, PRM1, TNP1, LELP1, HMGB4, AKAP4, CETN1, UBQLN3, ACTL7A, ACTL9, ACTRT2, PGK2, C2orf53, KIF21B, ADAD1, SPATA8, CCDC70, TPD52L3, ACTL71B, DMRTB1, SYCN, CELA2A, CELA21B, PNLIPRP1, CTRC-AMY2A, SERPINI2, RBPJL, AQP12A, IAPP, KIRREL2, G6PC2, AQP12B, CYP11B1, CYP11B2, STAR, CYP11A1, and MC2R in a number of different normal tissues and tumors, respectively.

As illustrated in the plots, these gene were identified as specific to the respective tissues as indicated on the top of each plot.

Next, the sequences of the genes identified in the first step were scanned through by the same bioinformatic program to find short peptide sequences with high likelihood of being presented on a common MHC I allele. Table 1A summarizes the findings on tissue-specific antigens and their corresponding cancer type, in which the respective tissue-specific gene from which the respective tissue-specific antigen was identified has relatively high expression level.

To validate this exemplary approach, a bioinformatics approach was used to identify of tissue-specific antigens. Table 2 summarizes a list of tissue-specific antigens ranked based on use of two different exemplary, algorithms that predict binding affinity of the peptides to HLA molecules. As can be seen in Table 2, for each peptide, their rank ranges by both programs were comparable. The total number of peptides in the data set was 8,962.

TABLE 2
Predictions of candidate tissue-specific antigens using two different algorithms

| Peptide | SEQ ID | | Rank range peptides | Rank range peptides fall |
|---|---|---|---|---|
| Sequence | NO: | Allele | fall within (algorithm 1) | within (algorithm 2) |
| YAITFAMRL | 8711 | HLA- | 0001-0100 | 0001-0100 |
| | | C03:04 | | |
| SLYEHLVNL | 6852 | HLA- | 0001-0100 | 0001-0100 |
| | | A02:01 | | |
| HTYPFGWYQK | 7726 | HLA- | 0001-0100 | 0001-0100 |
| | | A11:01 | | |
| FAINSEMFL | 2980 | HLA- | 0001-0100 | 0001-0100 |
| | | C03:04 | | |
| RPAPPGRYL | 7008 | HLA- | 0001-0100 | 0001-0100 |
| | | B07:02 | | |
| FVISGVLTL | 1668 | HLA- | 0001-0100 | 0001-0100 |
| | | C03:04 | | |
| SSLYSYFQK | 7179 | HLA- | 0050-0150 | 0050-0150 |
| | | A11:01 | | |
| RLFFRVCLK | 2641 | HLA- | 0050-0150 | 0050-0150 |
| | | A03:01 | | |
| VLTSGIVFV | 1665 | HLA- | 0150-0250 | 0150-0250 |
| | | A02:01 | | |
| YAGNNMHSL | 2066 | HLA- | 0200-0300 | 0200-0300 |
| | | C03:04 | | |
| RLFRTKTWK | 2229 | HLA- | 0250-0350 | 0250-0350 |
| | | A03:01 | | |
| VASDMMVSL | 575 | HLA- | 0300-0400 | 0300-0400 |
| | | C03:04 | | |
| YRKPGISLL | 8045 | HLA- | 0350-0450 | 0350-0450 |
| | | C07:02 | | |
| RLYFGTSGY | 7725 | HLA- | 0400-0500 | 0400-0500 |
| | | A03:01 | | |
| FIANLPPEL | 6601 | HLA- | 0400-0500 | 0400-0500 |
| | | C03:04 | | |
| SYPPLHEWAF | 4489 | HLA- | 0450-0550 | 0450-0550 |
| | | A24:02 | | |
| FAGILTTVL | 8323 | HLA- | 0550-0650 | 0550-0650 |
| | | C03:04 | | |
| VLDGLDVLL | 6098 | HLA- | 0800-0900 | 0800-0900 |
| | | A02:01 | | |
| YRYLCISHL | 7252 | HLA- | 1100-1200 | 1100-1200 |
| | | C07:02 | | |
| YMIPAKTLVQV | 2220 | HLA- | 1100-1200 | 1100-1200 |
| | | A02:01 | | |
| CVDAEGMEVY | 7629 | HLA- | 1300-1400 | 1300-1400 |
| | | A01:01 | | |
| HLMQKFEKV | 7691 | HLA- | 1350-1450 | 1350-1450 |
| | | A02:01 | | |
| IAHDLRLLL | 1262 | HLA- | 1550-1650 | 1550-1650 |
| | | C03:04 | | |
| VTLPPFMCNK | 7382 | HLA- | 1650-1750 | 1650-1750 |
| | | A03:01 | | |
| SSFSTTINY | 4573 | HLA- | 1800-1900 | 1800-1900 |
| | | A11:01 | | |
| LSDQAVLAL | 287 | HLA- | 1850-1950 | 1850-1950 |
| | | C03:04 | | |
| FLQQPEDLV | 3335 | HLA- | 2000-2100 | 2000-2100 |
| | | A02:01 | | |
| NYLSLPRPR | 1229 | HLA- | 2100-2200 | 2100-2200 |
| | | A33:03 | | |
| LYRDTGLVK | 1909 | HLA- | 2150-2250 | 2150-2250 |
| | | A30:01 | | |
| FSGNQVWRY | 4916 | HLA- | 2150-2250 | 2150-2250 |
| | | A01:01 | | |
| MRLCWAWEL | 1074 | HLA- | 2350-2450 | 2350-2450 |
| | | C07:01 | | |
| YAYASGNL | 6947 | HLA- | 2450-2550 | 2450-2550 |
| | | C03:04 | | |
| DIFIVYDTR | 8250 | HLA- | 2600-2700 | 2600-2700 |
| | | A33:03 | | |
| STFTETTLY | 7635 | HLA- | 2600-2700 | 2600-2700 |
| | | A01:01 | | |
| AMILPSSSL | 2185 | HLA- | 2750-2850 | 2750-2850 |
| | | C03:04 | | |
| TMDDDTAVLV | 5305 | HLA- | 2750-2850 | 2750-2850 |
| | | A02:01 | | |
| TFFFDPENF | 2243 | HLA- | 2800-2900 | 2800-2900 |
| | | A24:02 | | |
| RLHDGIADI | 4931 | HLA- | 2850-2950 | 2850-2950 |
| | | A02:01 | | |
| YRAYYTFLNF | 2999 | HLA- | 3100-3200 | 3100-3200 |
| | | C07:02 | | |
| SYKSFESDK | 5233 | HLA- | 3200-3300 | 3200-3300 |
| | | A30:01 | | |
| AYYTFLNFM | 3005 | HLA- | 3250-3350 | 3250-3350 |
| | | C07:02 | | |
| GPQNDGKQL | 7403 | HLA- | 3500-3600 | 3500-3600 |
| | | B07:02 | | |
| NIKHHYCYV | 227 | HLA- | 3500-3600 | 3500-3600 |
| | | A30:01 | | |
| NRGQRLLAF | 1360 | HLA- | 3500-3600 | 3500-3600 |
| | | C07:02 | | |
| APDMSRML | 1518 | HLA- | 4300-4400 | 4300-4400 |
| | | B07:02 | | |
| IYAGNNMHSL | 2016 | HLA- | 4400-4500 | 4400-4500 |
| L | | A24:02 | | |
| RALGFEPRK | 1591 | HLA- | 4400-4500 | 4400-4500 |
| | | A03:01 | | |
| VPNKALEL | 908 | HLA- | 4450-4550 | 4450-4550 |
| | | B07:02 | | |
| SAWSPTLPL | 7511 | HLA- | 4600-4700 | 4600-4700 |
| | | C01:02 | | |
| SSCPPQPCTK | 4298 | HLA- | 4600-4700 | 4600-4700 |
| | | A30:01 | | |
| GPGSGPLLRL | 2042 | HLA- | 4700-4800 | 4700-4800 |
| | | B07:02 | | |
| SVSESDTIR | 4077 | HLA- | 4800-4900 | 4800-4900 |
| | | A33:03 | | |
| SVVQVAKAT | 3039 | HLA- | 4950-5050 | 4950-5050 |
| GK | | A11:01 | | |
| FQQELALLK | 5025 | HLA- | 5150-5250 | 5150-5250 |
| | | A03:01 | | |
| RALCVIALLV | 1711 | HLA- | 5200-5300 | 5200-5300 |
| | | B13:02 | | |
| LTSGIVFVI | 1712 | HLA- | 5300-5400 | 5300-5400 |
| | | B13:02 | | |
| LAFLVLEAV | 8800 | HLA- | 5350-5450 | 5350-5450 |
| | | B13:02 | | |
| FTREFLGAL | 6759 | HLA- | 5600-5700 | 5600-5700 |
| | | B46:01 | | |
| DRRCQLNIL | 5594 | HLA- | 5750-5850 | 5750-5850 |
| | | C07:01 | | |
| RLFDDDETGK | 1583 | HLA- | 6200-6300 | 6200-6300 |
| I | | A02:01 | | |
| AAMANPRAL | 8920 | HLA- | 6250-6350 | 6250-6350 |
| | | B46:01 | | |
| LFHPEDTGQV | 3827 | HLA- | 6450-6550 | 6450-6550 |
| F | | A24:02 | | |
| QLSEEQNTGI | 5480 | HLA- | 6550-6650 | 6550-6650 |
| | | A02:01 | | |
| FTSFQYPEF | 8324 | HLA- | 6600-6700 | 6600-6700 |
| | | B46:01 | | |
| LSDYKEKQIL | 5721 | HLA- | 6950-7050 | 6950-7050 |
| KV | | A01:01 | | |
| LAIPLTDVKF | 8330 | HLA- | 6950-7050 | 6950-7050 |
| | | B46:01 | | |
| PSCLKKLLQR | 4318 | HLA- | 7200-7300 | 7200-7300 |
| | | A33:03 | | |
| CQPSCLKKL | 4287 | HLA- | 7250-7350 | 7250-7350 |
| | | C07:01 | | |
| FSRAVAAKW | 6763 | HLA- | 7300-7400 | 7300-7400 |
| | | B46:01 | | |
| HRDFSGHPNF | 5674 | HLA- | 7350-7450 | 7350-7450 |
| | | C04:01 | | |
| MSATTVSSL | 1932 | HLA- | 7550-7650 | 7550-7650 |
| | | C04:01 | | |
| KLLQRCFEK | 4285 | HLA- | 7800-7900 | 7800-7900 |
| | | A02:01 | | |
| KLGFKVTLP | 7377 | HLA- | 7800-7900 | 7800-7900 |
| | | A02:01 | | |
| GVACKGREQL | 7283 | HLA- | 8200-8300 | 8200-8300 |
| | | A02:01 | | |
| ACWPAFTVL | 1885 | HLA- | 8300-8400 | 8300-8400 |
| | | A24:02 | | |
| RHNVICQLL | 5775 | HLA- | 8300-8400 | 8300-8400 |
| | | B07:02 | | |
| RAMRCCRPRY | 6370 | HLA- | 8450-8550 | 8450-8550 |
| RP | | B13:02 | | |
| RHNVICQL | 5868 | HLA- | 8600-8700 | 8600-8700 |
| | | B07:02 | | |
| SKSRSPHKGV | 8445 | HLA- | 8750-8850 | 8750-8850 |
| | | A02:01 | | |
| RYYRQRQRS | 6327 | HLA- | 8800-8900 | 8800-8900 |
| | | A02:01 | | |
| SCQTRRRAM | 6323 | HLA- | 8850-8950 | 8850-8950 |
| | | A02:01 | | |

### Example 2. Confirmation of HLA-Binding Affinity and Immunogenicity

The following example demonstrates quantification of binding affinities of HLA class I and class II peptides (HLA binding assays), and test of the ability of each test peptide to expand T cells (immunogenicity assays). Experimental protocol described below are exemplary and non-limiting, other protocols following similar principle can also be used to test HLA binding affinity and immunogenicity of the peptide as described herein.

HLA binding assays can be performed with peptides that are either motif-bearing or not motif-bearing. An exemplary detailed description of the protocol utilized to measure the binding stability of peptides to Class I MHC has been published (Harndahl et al. J Immunol Methods. 374:5-12, 2011). Briefly, synthetic genes encoding biotinylated MHC-I heavy and light chains are expressed in E. coli and purified from inclusion bodies using standard methods. The light chain (β2m) is radio-labeled with iodine (1251), and combined with the purified MHC-I heavy chain and peptide of interest at 18° C. to initiate pMHC-1 complex formation. These reactions are carried out in streptavidin coated microplates to bind th biotinylated MHC-T heavy chains to the surface and allow measurement of radiolabeled light chain to monitor complex formation. Dissociation is initiated by addition of higher concentrations of unlabeled light-chain and incubation at 37° C. Stability is defined as the length of time in hours it takes for half of the complexes to dissociate, as measured by scintillation counts

Live cell/flow cytometry-based assays can also be used, e.g., an assay utilizing a TAP-deficient hybridoma cell line T2 (American Type Culture Collection (ATCC Accession No. CRL-1992). Manassas, Va.). TAP deficiency in this cell line leads to inefficient loading of MHCI in the ER and an excess of empty MHCIs. Salter and Cresswell, EMBO J. 5:943-49 (1986); Salter, Immunogenetics 21:23546 (1985). Empty MHCIs are highly unstable and short-lived. When T2 cells are cultured at reduced temperatures, empty MHCIs appear transiently on the cell surface, where they can be stabilized by exogenous addition of MHCI-binding peptides. To perform this binding assay, peptide-receptive MHCIs were induced by culturing aliquots of 107 T2 cells overnight at 26° C. in serum free AIM-V medium alone, or in medium containing escalating concentrations (0.1 to 100 μM) of peptide. Cells were then washed twice with PBS, and subsequently incubated with a fluorescent tagged HLA-A0201-specific monoclonal antibody. BB7.2, to quantify cell surface expression. Samples were acquired on a FACS Calibur instrument (Becton Dickinson) and the mean fluorescence intensity (MFI) determined using the accompanying Cellquest software.

Immunogenicity assays are used to test the ability of each test peptide to expand T cells. Mature professional APCs are prepared for these assays in the following way. Monocytes are enriched from healthy human donor PBMCs using a bead-based kit (Miltenyi). Enriched cells are plated in GM-CSF and IL-4 to induce immature DCs. After 5 days, immature DCs are incubated at 37° C. with each peptide for 1 hour before addition of a cytokine maturation cocktail (GM-CSF, IL-1β, IL-4, IL-6, TNFα, PGE1β). Cells are incubated at 37° C. to mature DCs.

After maturation of DCs. PBMCs (either bulk or enriched for T cells) are added to mature dendritic cells with proliferation cytokines. Cultures are monitored for peptide-specific T cells using a combination of functional assays and/or tetramer staining. Parallel immunogenicity assays with the modified and parent peptides allow for comparisons of the relative efficiency with which the peptides expanded peptide-specific T cells.

Tetramer Staining. MHC tetramers are used to measure peptide-specific T cell expansion in the immunogenicity assays. For the assessment, tetramer is added to 1×10⁵ cells in PBS containing 1% FCS and 0.1% sodium azide (FACS buffer) according to manufacturer's instructions. Cells are incubated in the dark for 20 minutes at room temperature. Antibodies specific for T cell markers, such as CD8, are then added to a final concentration suggested by the manufacturer, and the cells are incubated in the dark at 4° C., for 20 minutes. Cells are washed with cold FACS buffer and resuspended in buffer containing 1% formaldehyde. Cells are acquired on a FACS Calibur (Becton Dickinson) instrument, and are analyzed by use of Cellquest software (Becton Dickinson). For analysis of tetramer positive cells, the lymphocyte gate is taken from the forward and side-scatter plots. Data are reported as the percentage of cells that were CD8⁺/Tetramer⁺.

Intracellular cytokine staining. In the absence of well-established tetramer staining to identify antigen-specific T cell populations, antigen-specificity can be estimated using assessment of cytokine production using well-established flow cytometry assays. Briefly, T cells are stimulated with the peptide of interest and compared to a control. After stimulation, production of cytokines by CD4⁺ T cells (e.g., IFNγ and TNFα) are assessed by intracellular staining. These cytokines, especially IFNγ, used to identify stimulated cells.

EISPOT.

Peptide-specific T cells are functionally enumerated using the ELISPOT assay (BD Biosciences), which measures the release of IFNgamma from T cells on a single cell basis. Target cells (T2 or HLA-A0201 transfected C1Rs) were pulsed with 10 uM peptide for 1 hour at 37° C., and washed three times. 1×10⁵ peptide-pulsed targets are co-cultured in the ELISPOT plate wells with varying concentrations of T cells (5×10² to 2×10³) taken from the immunogenicity culture. Plates are developed according to the manufacturer's protocol, and analyzed on an ELISPOT reader (Cellular Technology Ltd.) with accompanying software. Spots corresponding to the number of IFNgamma-producing T cells are reported as the absolute number of spots per number of T cells plated. T cells expanded on modified peptides are tested not only for their ability to recognize targets pulsed with the modified peptide, but also for their ability to recognize targets pulsed with the parent peptide.

CD107 Staining.

CD107a and b are expressed on the cell surface of CD8⁺ T cells following activation with cognate peptide. The lytic granules of T cells have a lipid bilayer that contains lysosomal-associated membrane glycoproteins (“LAMPs”), which include the molecules CD107a and b. When cytotoxic T cells are activated through the T cell receptor, the membranes of these lytic granules mobilize and fuse with the plasma membrane of the T cell. The granule contents are released, and this leads to the death of the target cell. As the granule membrane fuses with the plasma membrane, C107a and b are exposed on the cell surface, and therefore are markers of degranulation. Because degranulation as measured by CD107 a and b staining is reported on a single cell basis, the assay is used to functionally enumerate peptide-specific T cells. To perform the assay, peptide is added to HLA-A0201-transfected cells C1R to a final concentration of 20 μM, the cells were incubated for 1 hour at 37° C., and washed three times. 1×10⁵ of the peptide-pulsed CIR cells were aliquoted into tubes, and antibodies specific for CD107 a and b are added to a final concentration suggested by the manufacturer (Becton Dickinson). Antibodies are added prior to the addition of T cells in order to “capture” the CD107 molecules as they transiently appear on the surface during the course of the assay, 1×10⁵ T cells from the immunogenicity culture are added next, and the samples were incubated for 4 hours at 37° C. The T cells are further stained for additional cell surface molecules such as CD8 and acquired on a FACS Calibur instrument (Becton Dickinson). Data is analyzed using the accompanying Cellquest software, and results were reported as the percentage of CD8⁺ CD107 a and b⁺ cells.

Cytotoxicity Assays.

Cytotoxic activity is measured using a chromium release assay. Target T2 cells are labeled for 1 hour at 37° C. with Na⁵¹Cr and washed 5×10³ target T2 cells were then added to varying numbers of T cells from the immunogenicity culture. Chromium release is measured in supernatant harvested after 4 hours of incubation at 37° C. The percentage of specific lysis is calculated as:

Experimental release-spontaneous release/Total release-spontaneous release×100.

### Example 3. Selection of Tissue-Specific Antigens for a Tumor-Specific Vaccine

This example illustrates the procedure for the selection of peptide epitopes for vaccine compositions of the invention. The peptides in the composition can be in the form of a nucleic acid sequence, either single or one or more sequences (i.e., minigene) that encodes peptide(s), or may be single and/or polyepitopic peptides.

Epitopes are selected which, upon administration, mimic immune responses that have been observed to be correlated with tumor clearance. For example, vaccine can include 1-2 epitopes that come from at least one tissue-specific antigen region. Epitopes from one region can be used in combination with epitopes from one or more additional tissue-specific antigen regions.

Epitopes can be selected, for example, that have a binding affinity of an IC₅₀ of 500 nM or less for an HLA class I molecule, or for class II, an IC₅₀ of 1000 nM or less.

When creating a polyepitopic compositions, e.g. a minigene, it is typically desirable to generate the smallest peptide possible that encompasses the epitopes of interest. The principles employed are similar, if not the same, as those employed when selecting a peptide comprising nested epitopes. Additionally, however, upon determination of the nucleic acid sequence to be provided as a minigene, the peptide sequence encoded thereby is analyzed to determine whether any “junctional epitopes” have been created. A junctional epitope is a potential HLA binding epitope, e.g., as predicted by motif analysis. Junctional epitopes are generally to be avoided because the recipient may bind to an HLA molecule and generate an immune response to that epitope, which is not present in a native protein sequence.

Peptide epitopes for inclusion in vaccine compositions are, for example, selected from those listed in the Tables. A vaccine composition comprised of selected peptides, when administered, is safe, efficacious, and elicits an immune response similar in magnitude of an immune response that inhibits tumor growth.

### Example 4. Composition for Prophylactic or Therapeutic Uses

Immunogenic or vaccine compositions of the present disclosure are used to inhibit tumor growth. For example, a polyepitopic composition (or a nucleic acid comprising the same) containing multiple tissue-specific epitopes is administered to individuals having tumors. The dose of peptide for the immunization is from about 1 to about 50,000 μg, generally 100-5,000 μg, for a 70 kg patient. The initial administration may be followed by booster dosages at 4 weeks followed by evaluation of the magnitude of the immune response in the patient, by techniques that determine the presence of epitope-specific CTL populations in a PBMC sample. Additional booster doses are administered as required. The composition is found to be both safe and efficacious to inhibit tumor growth.

Alternatively, the polyepitopic composition can be administered as a nucleic acid, for example as RNA, in accordance with methodologies known in the art and disclosed herein.

Tissue-specific antigen binding agents, such as TCR or CARs can be administered in accordance with methodologies known in the art and disclosed herein. The binding agents can be administered as polynucleotides, for example DNA or RNA, encoding the binding agents as part of cellular therapy. Alternatively, the binding agents can be prepared as antibodies or fragments thereof capable of recognizing the specific peptide: MHC complex coupled to cytotoxic agents or T cell binding agents capable of re-directing patient T cells to tumor cells expressing the epitopes listed in the Tables.

Tissue-specific antigen peptides, polynucleotides, binding agents, or cells expressing these molecules can be delivered to the same patient via multiple methodologies known in the art, and can further be combined with other cancer therapies (e.g., chemotherapy, surgery, radiation, checkpoint inhibitors, etc.).

### Example 5. Identification of Tissue-Specific Antigen

This example illustrates an exemplary process for identification of tissue-specific antigen.

Step 1.

RNA-Seq-based data were acquired from GTEx and TCGA. Expression was merged (by summation) to the gene symbol level (considering protein-coding genes only), and each sample was scaled such that its values summed to 1,000,000. These values represent transcripts per million (TPM).

Step 2.

Genes were identified as being expressed highly in cancer and weakly expressed or absent in essential tissues. Implicitly, genes highly expressed in cancer and non-essential tissues (but not in essential tissues) were still considered as valid targets). The tissues listed in Table 3A were deemed as ESSENTIAL. The tissues in Table 3B were used to represent tumors.

TABLE 3A
From GTEx:

| | |
|---|---|
| Adipose - Subcutaneous | Colon - Transverse |
| Adipose - Visceral (Omentum) | Esophagus - Gastroesophageal Junction |
| Artery - Aorta | Esophagus - Mucosa |
| Artery - Coronary | Esophagus - Muscularis |
| Artery - Tibial | Heart - Atrial Appendage |
| Bladder | Heart - Left Ventricle |
| Brain - Amygdala | Kidney - Cortex |
| Brain - Anterior cingulate corte | Liver |
| Brain - Caudate (basal ganglia) | Lung |
| Brain - Cerebellar Hemisphere | Minor Salivary Gland |
| Brain - Cerebellum | Muscle - Skeletal |
| Brain - Cortex | Nerve - Tibial |
| Brain - Frontal Cortex (BA9) | Pituitary |
| Brain - Hippocampus | Skin - Not Sun Exposed (Suprapubic) |
| Brain - Hypothalamus | Skin - Sun Exposed (Lower leg) |
| Brain - Nucleus accumbens | Small Intestine - Terminal Ileum |
| Brain - Putamen (basal ganglia) | Spleen |
| Brain - Spinal cord (cervical c-1) | Stomach |
| Brain - Substantia nigra | Uterus |
| EBV-transformed lymphocytes | Vagina |
| Cells - Transformed fibroblasts | Whole Blood |
| Colon - Sigmoid | |

From TCGA (non-tumor normal samples):

| | |
|---|---|
| Bladder | Liver |
| Brain | Skin |
| Colon | Stomach |
| Throat (“head and neck”) | Non-smoker Lung |
| Kidney | |

TABLE 3B
From GTEx (normal tissues used as surrogates for tumor tissues):

| | |
|---|---|
| Adrenal Gland | Testis |
| Pancreas | |

From TCGA (tumor tissues):

| | |
|---|---|
| Bladder cancer | Liver cancer |
| Breast cancer | Lung adenocarcinoma |
| Cervical cancer | Lung squamous cell carcinoma |
| Colorectal cancer | Melanoma |
| Glioblastoma | Ovarian cancer |
| Glioma | Prostate cancer |
| Head&neck cancer | Stomach cancer |
| Kidney clear cell cancer | Thyroid cancer |
| Kidney papillary cancer | Uterine cancer |

The following calculations were carried out to select candidate genes:
i. For each combination of essential tissue (tissues listed in Table 2A above) and gene, the 95th percentile value expression value was calculated across available samples (using quantile function in R, default parameters, as described in R Core Team (2015). R: A language and environment for statistical computing. R Foundation for Statistical Computing, Vienna, Austria). This was then summarized to a single value per gene using a max operation across different tissues, which was referred to as the “essential expression” of each gene. The initial set of candidate genes comprised all genes with essential expression less than 20 TPM.
ii. For each combination of tumor tissue (tissues listed in Table 2B above) and candidate gene, the 75th percentile expression value was calculated across available samples (also using quantile function in R). If this values was at least 10 times greater than the gene's essential expression, then the gene was considered as a candidate for the given tumor type.

Step 3.

For each gene with suitably restricted expression, all the protein coding sequences of all distinct transcript isoforms (per the Gencode V19 annotation) were digested (in silico) into all possible peptides of lengths 8, 9, 10, 11, and 12. If a peptide was also found in the protein sequence of a gene with an essential score greater than 20 (as might happen in the case of gene paralogs pairs for which one gene has restricted expression and the other does not) then the peptide was excluded as a candidate. The remaining candidate peptides were scored for binding potential using NetMHCpan-v3.0 and RECON for the following HLA I alleles:

TABLE 4

| HLA-A | HLA-B | HLA-C |
|---|---|---|
| HLA-A02:01 | HLA-B07:02 | HLA-C07:01 |
| HLA-A01:01 | HLA-B08:01 | HLA-C07:02 |
| HLA-A03:01 | HLA-B13:02 | HLA-C04:01 |
| HLA-A24:02 | HLA-B46:01 | HLA-C01:02 |
| HLA-A11:01 | — | HLA-C07:02 |
| HLA-A24:02 | — | HLA-C03:04 |
| HLA-A02:01 | — | — |
| HLA-A33:03 | — | — |
| HLA-A30:01 | — | — |

Step 4.

For each combination of gene and allele, a peptide was considered to be a positive hit if its predicted binding (per NetMHCpan3.0 or RECON) placed it in the N top-scoring peptides. N was calculated as max(3,0.001*P), where P is the number of peptides evaluated for the given gene-allele combination.

### Example 6. T Cell Manufacturing Protocol 1

This example provides an example of T cell manufacturing protocol.

Materials:
DC media (Cellgenix)
CD14 microbeads, human, Miltenyi #130-050-201
Cytokines and/or growth factors
T cell media (AIM V+RPMI 1640 glutamax⁺ serum+PenStrep)
Peptide stocks—1 mM per peptide (HIV A02—5-10 peptides, HIV B07—5-10 peptides,
DOM—4-8 peptides, PIN—6-12 peptides)

Step 1: Monocyte Isolation for DC Prep
1. Calculate the approximate number of PBMCs to thaw based on expected DC yield for each donor.
2. Thaw PBMCs and resuspend at ˜1×10⁶-1×10⁸ cells/mL in DC media.
3. Add benzonase (1:1000 dilution) and place in incubator with cap loosened.
4. Perform CD14⁺ monocyte enrichment according to manufacturer protocol.
5. Plate enriched cells in 6-well plates at 1×10⁵-1×10⁷ per well in DC media with one or more cytokines and/or growth factors selected from GM-CSF, IL-4, FLT3L, TNF-α, IL-1β, PGE1, IL-6, TL-7, IFN-α, R848, LPS, ss-rna40, and polyI:C.

Step 2: Peptide Loading and Maturation
1. Count DCs and split the cells according to the experimental conditions in 15 mL tubes, 0.01-1 million cells per condition.
2. Spin id 1200 rpm for 5 min and resuspend in 50-400 μL DC medium. Add peptide(s) and place in incubator with loosened cap for 0.5-3 hrs. Volumes were calculated for peptide pools at a concentration of 1 mM per peptide. A volume of each separate pool of A02 (5 peptides) and B07 (5 peptides) was added per well for a final concentration of 0.001-100 μM per peptide.
3. After 0.5-3 hrs add 200 μL to 1.5 mL of DC media containing maturation mix and transfer the cells to 24 well plate.
   The maturation mix contains one or more cytokines selected from GM-CSF, IL-4, FLT3L, TNF-α, IL-1β, PGE1, IL-6, IL-7, IFN-α, R848, LPS, ss-ma40, and polyI:C.

Step 3: Setting Up Long Term Stimulation (LTS) Experiment
1. Carefully remove all media from the wells of the DC plates, transferring each well to a separate well in a 24-well deepwell block.
2. Wash each well with 0.5-3 mL T cell media and combine with DC media in the deepwell block.
3. Add 100 μL to 2 mL T cell media to each well.
4. Spin down DCs at 1200 rpm for 5 min.
5. Remove all supernatant, resuspend DCs in 100 μL to 2 mL T cell media and transfer back into the correct wells.
6. Thaw PBMCs in T cell media and resuspend at 0.5×10⁶-4×10⁶ cells/mL in T cell media with IL-7 and IL-15.
7. Add 0.5-3 mL of prepared PBMCs to each well.

Step 4: Feeding LTS
Check with glucose meter if the media is yellow. If glucose remains high, feed culture with IL-7 and IL-15 to the well. If glucose is low, expand the cells to 6 well plate (4 mL/well) and supplement with IL-15 and IL-7. If glucose is very low, expand to 6 mL/well in a 6-well plate.

Step 5: Feeding LTS
Feed cultures every 1-4 days, adding fresh IL-15/IL-7 and expanding the culture volume as needed when glucose concentration becomes low.

Step 6: Re-Stimulation
Count T cells and repeat from step 3 on a new batch of peptide-loaded DCs. Freeze leftover cells for analysis.

Step 7: Feeding LTS
Feed cultures every −1-5 days.

Step 8: Re-Stimulation
Count T cells and repeat from step 3 on a new batch of peptide-loaded DCs. Freeze leftover cells for analysis.

Step 9: Feeding LTS
Feed cultures every 1-5 days.

Step 10
Count T cells and freeze for analysis.

### Example 7. T Cell Manufacturing Protocol 2

This protocol can be an alternative to the protocol described in Example 6.

Materials:
AIM V media (Invitrogen)
Media 1 (RPMI 1640 glutamax+serum+PenStrep)
Media 2 (AIM V+RPMI 1640 glutamax+serum+PenStrep)

Procedure:
Step 1: Plate 4 million PBMCs in each well of 24 well plate with one or more cytokines in Media 2. The one or more cytokines are selected from GM-CSF, IL-4, FLT3L, TNF-α, IL-1β, PGE1, IL-6, IL-7, IFN-α, R848, LPS, ss-rna40, and polyI:C.
Step 2: Peptide loading and maturation in Media 2
1. Make stock peptide pool of interest (except for no peptide condition) at 0.001-100 μM for shortmers and 0.001-100 μM for longmers final concentration in respective wells and mix.
2. Incubate for 0.5-3 hr.
3. Make stock maturation cocktail and add to each well after incubation and mix. The maturation cocktail contains one or more cytokines selected from GM-CSF, IL-4, FLT3L, TNF-α, IL-1β, PGE1, IL-6, IL-7, IFN-α, R848, LPS, ss-rna40, and polyI:C.
Step 3: Add human serum to each well at a final concentration of 2.5-20% by volume and mix.
Step 4: Carefully replace 50-90% of the media with fresh Media I supplemented with IL-7 and IL-15 to a final concentration of 0.005-500 ng/mL each.
Step 5: Carefully replace 50-90% of the media with fresh Media I supplemented with IL-7 and IL-15 to a final concentration of 0.005-500 ng/mL each every 1-5 days.
In case the wells turn orange to yellow on non-feeding days (glucose readout in case of clear media), change 25-75% of existing media with fresh Media 1 and IL-7/IL-15.
Step 6: Count and freeze (or proceed to the following steps to carry the T cell simulation to step 8 and/or step 10 of protocol 1).
During the culturing steps from step 1 to step 6, peptide-loaded DCs can be prepared in parallel according to the procedures in protocol 1 “Step I” and “Step 2”.
Count T cells and stimulate T cells with a new batch of peptide-loaded DCs. Freeze leftover cells for analysis. The T cell stimulation procedure can be carried out according to the procedures in protocol 1 “Step 3”.
Step 7: Count T cells and repeat T cell stimulation procedures in protocol 1 “step 3” on a new batch of peptide-loaded DCs. Freeze leftover cells for analysis.
Step 8: Count T cells and freeze for analysis.

### Example 8. T Cell Manufacturing Protocol 3

Materials:
AIM V media (Invitrogen)
Human FLT3L, preclinical CellGenix #1415-050 Stock 50 ng/μL
TNF-α, preclinical CellGenix #1406-050 Stock 10 ng/μL
IL-1β, preclinical CellGenix #1411-050 Stock 10 ng/μL
PGE1 or Alprostadil—Cayman from Czech republic Stock 0.5 μg/μL
R10 media-RPMI 1640 glutamax+10% Human serum+1% PenStrep
20/80 Media-18% AIM V+72% RPMI 1640 glutamax+10% Human Serum+1% PenStrep
IL7 Stock 5 ng/μL
IL15 Stock 5 ng/μL

Procedure:

Step 1: Plate 5 million PBMCs (or cells of interest) in each well of 24 well plate with FLT3L in 2 mL AIM V media

Step 2: Peptide loading and maturation-in AIMV
1. Mix peptide pool of interest (except for no peptide condition) with PBMCs (or cells of interest) in respective wells.
2. Incubate for 1 hr.
3. Mix Maturation cocktail (including TNF-α, IL-1β, PGE1, and IL-7) to each well after incubation.
Step 3: Add human serum to each well at a final concentration of 10% by volume and mix.
Step 4: Replace the media with fresh RPMI+10% HS media supplemented with 1L7+IL15.
Step 5: Replace the media with fresh 20/80 media supplemented with IL7+IL 15 during the period of incubation every 1-6 days.
Step 6: Plate 5 million PBMCs (or cells of interest) in each well of new 6-well plate with FLT3L in 2 mL AIM V media
Step 7: Peptide loading and maturation for re-stimulation-(new plates)
1. Mix peptide pool of interest (except for no peptide condition) with PBMCs (or cells of interest) in respective wells 2. Incubate for 1 hr.
3. Mix Maturation cocktail to each well after incubation
Step 8: Re-stimulation:
1. Count first stimulation FLT3L cultures and add 5 million cultured cells to the new Re-stimulation plates.
2. Bring the culture volume to 5 mL (AIM V) and add 500 ul of Human serum (10% by volume)
Step 9: Remove 3 mL of the media and add 6 mL of RPMI+10% HS media supplemented with 1L7+IL15.
Step 10: Replace 75% of the media with fresh 20/80 media supplemented with IL7+IL 15.
Step 11: Repeat re-stimulation if needed.

### Example 9. T Cell Induction Protocol

T Cell Induction #1

| | |
|---|---|
| Step 7 | Autologous Cells |
| | Apheresis Bag #2 |
| Step 8 | CD25⁺ depletion (+/−CD14⁺ depletion) |
| Step 8a | Add FLT3L |
| Step 9 | Addition Patient Specific Peptides and incubation |
| Step 10 | Incubation of depleted PMBCs with FLT3L and peptides |

### T Cell Induction #2

| | |
|---|---|
| Step 11 | T Cell Washing and Resuspension in T cell Media |
| Step 12 | Incubation of T cells with Matured DC (from DC Derivation) |

### T Cell Induction #3

| | |
|---|---|
| Step 11 | T Cell Washing and Resuspension in T cell Media |
| Step 12 | Incubation of T cells with Matured DC (from DC Derivation) |

### Harvest & Cryopreservation

| | | | |
|---|---|---|---|
| Step 15 | | T Cell Harvest | Release Testing: |
| | | | Mycoplasma |
| Step 1 | Drug | Wash and Suspension | Release Testing: Sterility, |
| | Substance | in Final Formulation | Endotoxin, Cell Phenotype, |
| | (DS) | | TNC Count, Viability, Cell |
| | | | Concentration, Potency |
| Step 17 | Drug | DS Fill and | |
| | Product | Cryopreservation | |
| | (DP) | Store in vapor phase | |
| | | of liquid nitrogen | |

### Example 10. T Cell Manufacturing

Provided herein is a T cell therapy where T cells primed and responsive against antigenic peptides specific for a tissue-specific epitope is administered to the subject. Provided herein are methods for generating tissue-specific epitope responsive T cells for the therapy. The method can comprise generating tissue-specific epitope responsive T cells ex vivo by priming T cells with APCs expressing tissue-specific T cell epitopes and expanding the activated T cells to obtain tissue-specific epitope responsive CD8+ and CD4+ including a population of these cells exhibiting memory phenotype (see, e.g., WO2019094642, incorporated by reference in its entirety). Target tissue-specific antigen responsive T cells are generated ex vivo and immunogenicity is validated using an in vitro antigen-specific T cell assay. Mass spectrometry can be used to validate that cells that express the antigen of interest can process and present the peptides on the relevant HLA molecules. Additionally, the ability of these T cells to kill cells presenting the peptide is confirmed using a cytotoxicity assay.

### Generation of Target Tumor Cell Antigen Responsive T Cells Ex Vivo

Materials:
AIM V media (Invitrogen)
Human FLT3L, preclinical CellGenix #1415-050 Stock 50 ng/μL
TNF-α, preclinical CellGenix #1406-050 Stock 10 ng/μL
IL-1β, preclinical CellGenix #1411-050 Stock 10 ng/μL
PGE1 or Alprostadil—Cayman from Czech republic Stock 0.5 μg/μL
R10 media-RPMI 1640 glutamax+10% Human serum+1% PenStrep
20/80 Media-18% AIM V+72% RPMI 1640 glutamax+10% Human Serum+1% PenStrep
IL7 Stock 5 ng/μL
IL 15 Stock 5 ng/μL

### Procedure:

Step 1: Plate 5 million PBMCs (or cells of interest) in each well of 24 well plate with FLT3L in 2 mL AIM V media
Step 2: Peptide loading and maturation-in AIMV
1. Mix peptide pool of interest (except for no peptide condition) with PBMCs (or cells of interest) in respective wells.
2. Incubate for 1 hr.
3. Mix Maturation cocktail (including TNF-α, IL-1β, PGE1, and IL-7) to each well after incubation.
Step 3: Add human serum to each well at a final concentration of 10% by volume and mix.
Step 4: Replace the media with fresh RPMI+10% HS media supplemented with 1L7+IL15.
Step 5: Replace the media with fresh 20/80 media supplemented with IL7+IL15 during the period of incubation every 1-6 days.
Step 6: Plate 5 million PBMCs (or cells of interest) in each well of new 6-well plate with FLT3L in 2 ml AIM V media
Step 7: Peptide loading and maturation for re-stimulation-(new plates)
1. Mix peptide pool of interest (except for no peptide condition) with PBMCs (or cells of interest) in respective wells
2. Incubate for 1 hr.
3. Mix Maturation cock-tail to each well after incubation
Step 8: Re-stimulation:
1. Count first stimulation FLT3L cultures and add 5 million cultured cells to the new Re-stimulation plates.
2. Bring the culture volume to 5 mL (AIM V) and add 500 μL of Human serum (10% by volume) Step 9: Remove 3 mL of the media and add 6 mL of RPMI+10% HS media supplemented with IL7+IL 15.
Step 10: Replace 75% of the media with fresh 20/80 media supplemented with IL7+IL 15.
Step 11: Repeat re-stimulation if needed.

### Analysis of Antigen-Specific Induction

MHC tetramers are purchased or manufactured on-site according to methods known by one of ordinary skill and are used to measure peptide-specific T cell expansion in the immunogenicity assays. For the assessment, tetramer is added to 1×10⁵ cells in PBS containing 1% FCS and 0.1% sodium azide (FACS buffer) according to manufacturer's instructions. Cells are incubated in the dark for 20 minutes at room temperature. Antibodies specific for T cell markers, such as CD8, are then added to a final concentration suggested by the manufacturer, and the cells are incubated in the dark at 4° C. for 20 minutes. Cells are washed with cold FACS buffer and resuspended in buffer containing 1% formaldehyde. Cells are acquired on a LSR Fortessa (Becton Dickinson) instrument and are analyzed by use of FlowJo software (Becton Dickinson). For analysis of tetramer positive cells, the lymphocyte gate is taken from the forward and side-scatter plots. Data are reported as the percentage of cells that were CD8⁺/tetramer⁺.

### Evaluation of Presentation of Tissue-Specify Antigens

The affinity of the tissue-specific epitope s for HLA alleles and stability of the tissue-specific epitopes with the HLA alleles can be determined as described herein. An exemplary detailed description of the protocol utilized to measure the binding affinity of peptides to Class I MHC has been published (Sette et al, Mol. Immunol. 31(11):813-22, 1994). In brief, MHCI complexes were prepared and bound to radiolabeled reference peptides. Peptides were incubated at varying concentrations with these complexes for 2 days, and the amount of remaining radiolabeled peptide bound to MHCI was measured using size exclusion gel-filtration. The lower the concentration of test peptide needed to displace the reference radiolabeled peptide demonstrates a stronger affinity of the test peptide for MHCI. Peptides with affinities to MHCI<50 nM are generally considered strong binders while those with affinities <150 nM are considered intermediate binders and those <500 nM are considered weak binders (Fritsch et al, 2014).

An exemplary detailed description of the protocol utilized to measure the binding stability of peptides to Class I MHC has been published (Harndahl et al. J Immunol Methods. 374:5-12, 2011). Briefly, synthetic genes encoding biotinylated MHC-1 heavy and light chains are expressed in E. coli and purified from inclusion bodies using standard methods. The light chain (β2m) is radio-labeled with iodine (1251), and combined with the purified MHC-1 heavy chain and peptide of interest at 18° C. to initiate pMHC-I complex formation. These reactions are carried out in streptavidin coated microplates to bind the biotinylated MHC-I heavy chains to the surface and allow measurement of radiolabeled light chain to monitor complex formation. Dissociation is initiated by addition of higher concentrations of unlabeled light-chain and incubation at 37° C. Stability is defined as the length of time in hours it takes for half of the complexes to dissociate, as measured by scintillation counts.

To assess whether antigens could be processed and presented from the larger polypeptide context, peptides eluted from HLA molecules isolated from cells expressing the genes of interest were analyzed by tandem mass spectrometry (MS/MS).

For analysis of presentation of tissue-specific antigens, cell lines are utilized that were lentivirally transduced to express the tissue-specific antigens. HLA molecules are either isolated based on the natural expression of the cell lines or the cell lines are lentivirally transduced or transiently transfected to express the HLA of interest. 293T cells are transduced with a lentiviral vector encoding various regions of a tissue-specific polypeptides. Greater than 50 million cells expressing peptides encoded by a tissue-specific polypeptide are cultured and peptides were eluted from HLA-peptide complexes using an acid wash. Eluted peptides are then analyzed by targeted MS/MS with parallel reaction monitoring (PRM).

### HLA Class I Binding and Stability

A subset of the peptides used for affinity measurements are also used for stability measurements using the assay described. Less than 50 nM can be considered by the field as a strong binder, 50-150 nM can be considered an intermediate binder, 150-500 nM can be considered a weak binder, and greater than 500 nM can be considered a very weak binder.

Immunogenicity assays are used to test the ability of each test peptide to expand T cells. Mature professional APCs are prepared for these assays in the following way. Monocytes are enriched from healthy human donor PBMCs using a bead-based kit (Miltenyi). Enriched cells are plated in GM-CSF and IL-4 to induce immature DCs. After 5 days, immature DCs are incubated at 37° C. with each peptide for 1 hour before addition of a cytokine maturation cocktail (GM-CSF, IL-1β, IL-4, IL-6, TNFα, PGE1β). Cells are incubated at 37° C. to mature DCs.

### Assessment of Cytotoxic Capacity of Antigen-Specific T Cells In Vitro

Cytotoxicity activity can be measured with the detection of cleaved Caspase 3 in target cells by Flow cytometry. Target cancer cells are engineered to express the tissue-specific peptide along and the proper MHC-I allele. Mock-transduced target cells (i.e. not expressing the tissue-specific peptide) are used as a negative control. The cells are labeled with CFSE to distinguish them from the stimulated PBMCs used as effector cells. The target and effector cells are co-cultured for 6 hours before being harvested. Intracellular staining is performed to detect the cleaved form of Caspase 3 in the CFSE-positive target cells. The percentage of specific lysis is calculated as: Experimental cleavage of Caspase 3/spontaneous cleavage of Caspase 3 (measured in the absence of the specific peptide expression)×100.

In some examples, cytotoxicity activity is assessed by co-culturing induced T cells with a population of tissue-specific antigen-specific T cells with target cells expressing the corresponding HLA, and by determining the relative growth of the target cells, along with measuring the apoptotic marker Annexin V in the target cells specifically. Target cells are engineered to express the tissue-specific peptide or the tissue-specific peptide is exogenously loaded. Mock-transduced target cells (i.e. not expressing the tissue-specific peptide), target cells loaded with tissue-specific peptides, or target cells with no peptide loaded are used as a negative control. The cells are also transduced to stably express GFP allowing the tracking of target cell growth. The GFP signal or Annexin-V signal arc measured over time with an IncuCyte S3 apparatus. Annexin V signal originating from effector cells is filtered out by size exclusion. Target cell growth and death is expressed as GFP and Annexin-V area (mm²) over time, respectively.

### Enrichment of Target Antigen Activated T Cells

Tissue-specific antigen responsive T cells may be further enriched. In this example, multiple avenues for enrichment of antigen responsive T cells are explored. After the initial stimulation of tissue-specific antigen-specific T cells, an enrichment procedure can be used prior to further expansion of these cells. As an example, stimulated cultures and pulsed with the same tissue-specific peptides used for the initial stimulation on day 13, and cells upregulating 4-1BB are enriched using Magnetic-Assisted Cell Separation (MACS; Miltenyi). These cells can then be further expanded, for example, using anti-CD3 and anti-CD28 microbeads and low-dose IL-2.

### Immunogenicity Assays for Selected Peptides

After maturation of DCs, PBMCs (either bulk or enriched for T cells) are added to mature dendritic cells with proliferation cytokines. Cultures are monitored for tissue-specific peptide-specific T cells using a combination of functional assays and/or tetramer staining. Parallel immunogenicity assays with the tissue-specific peptides allowed for comparisons of the relative efficiency with which the peptides expanded peptide-specific T cells. In some embodiments, the peptides elicit an immune response in the T cell culture comprises detecting an expression of a FAS ligand, granzyme, perforins, IFN, TNF, or a combination thereof in the T cell culture.

Immunogenicity can be measured by a tetramer assay. MHC tetramers are purchased or manufactured on-site, and are used to measure peptide-specific T cell expansion in the immunogenicity assays. For the assessment, tetramer is added to 1×10\hat{ }5 cells in PBS containing 1% FCS and 0.1% sodium azide (FACS buffer) according to manufacturer's instructions. Cells are incubated in the dark for 20 minutes at room temperature. Antibodies specific for T cell markers, such as CD8, are then added to a final concentration suggested by the manufacturer, and the cells are incubated in the dark at 4 degrees Celsius for 20 minutes. Cells are washed with cold FACS buffer and resuspended in buffer containing 1% formaldehyde. Cells are acquired on a FACS Calibur (Becton Dickinson) instrument, and are analyzed by use of Cellquest software (Becton Dickinson). For analysis of tetramer positive cells, the lymphocyte gate is taken from the forward and side-scatter plots. Data are reported as the percentage of cells that were CD8⁺/Tetramer⁺.

Immunogenicity can be measured by intracellular cytokine staining. In the absence of well-established tetramer staining to identify tissue-specific antigen-specific T cell populations, antigen-specificity can be estimated using assessment of cytokine production using well-established flow cytometry assays. Briefly, T cells are stimulated with the tissue-specific peptide of interest and compared to a control. After stimulation, production of cytokines by CD4+ T cells (e.g., IFNγ and TNFα) are assessed by intracellular staining. These cytokines, especially IFNγ, used to identify stimulated cells.

In some embodiments the immunogenicity is measured by measuring a protein or peptide expressed by the T cell, using ELISpot assay. Peptide-responsive T cells are functionally enumerated using the ELISpot assay (BD Biosciences), which measures the release of IFNγ from T cells on a single cell basis. Target cells are pulsed with 10 μM tissue-specific peptide for one hour at 37 degrees C., and washed three times, 1×10\hat{ }5 peptide-pulsed targets are co-cultured in the ELISPOT plate wells with varying concentrations of T cells (5×10\hat{ }2 to 2×10\hat{ }3) taken from the immunogenicity culture. Plates are developed according to the manufacturer's protocol, and analyzed on an ELISPOT reader (Cellular Technology Ltd.) with accompanying software. Spots corresponding to the number of IFN gamma-producing T cells are reported as the absolute number of spots per number of T cells plated. T cells expanded on modified peptides are tested not only for their ability to recognize targets pulsed with the modified peptide, but also for their ability to recognize targets pulsed with the parent peptide.

CD107a and CD107b are expressed on the cell surface of CD8+ T cells following activation with tissue-specific peptide. The lytic granules of T cells have a lipid bilayer that contains lysosomal-associated membrane glycoproteins (“LAMPs”), which include the molecules CD107a and b. When cytotoxic T cells are activated through the T cell receptor, the membranes of these lytic granules mobilize and fuse with the plasma membrane of the T cell. The granule contents are released, and this leads to the death of the target cell. As the granule membrane fuses with the plasma membrane, C107a and b are exposed on the cell surface, and therefore are markers of degranulation. Because degranulation as measured by CD107a and b staining is reported on a single cell basis, the assay is used to functionally enumerate tissue-specific peptide-specific T cells. To perform the assay, peptide is added to HLA-transfected cells to a final concentration of 20 μM, the cells are incubated for 1 hour at 37 degrees C., and washed three times, 1×10\hat{ }5 of the peptide-pulsed cells were aliquoted into tubes, and antibodies specific for CD107a and b are added to a final concentration suggested by the manufacturer (Becton Dickinson). Antibodies are added prior to the addition of T cells in order to “capture” the CD107 molecules as they transiently appear on the surface during the course of the assay, 1×10\hat{ }5 T cells from the immunogenicity culture are added next, and the samples were incubated for 4 hours at 37 degrees C. The T cells are further stained for additional cell surface molecules such as CD8 and acquired on a FACS Calibur instrument (Becton Dickinson). Data is analyzed using the accompanying Cellquest software, and results are reported as the percentage of CD8+ CD107 a and b+ cells.

Cytotoxic activity is measured using a chromium release assay. Target T2 cells are labeled for 1 hour at 37 degrees C., with Na⁵¹Cr and washed 5×10\hat{ }3 target cells are then added to varying numbers of T cells from the immunogenicity culture. Chromium release is measured in supernatant harvested after 4 hours of incubation at 37 degrees C. The percentage of specific lysis is calculated as:

Experimental release-spontaneous release/Total release-spontaneous release×100

Immunogenicity assays are carried out to assess whether each peptide can elicit a T cell response by tissue-specific antigen-specific expansion. A positive result demonstrates that a peptide can induce a T cell response. Several tissue-specific peptides are tested for their capacity to elicit CD8+ T cell responses with multimer readouts as described. Each positive result was measured with a second multimer preparation to avoid any preparation biases. In an exemplary assay, T cells were co-cultured with monocyte-derived dendritic cells loaded with tissue-specific epitope for 10 days. CD8+ T cells were analyzed for tissue-specific antigen-specificity for tissue-specific epitope using multimers (initial: BV421 and PE: validation: APC and BUV396).

While antigen-specific CD8+ T cell responses are readily assessed using well-established HLA Class 1 multimer technology, CD4+ T cell responses require a separate assay to evaluate because HLA Class II multimer technology is not well-established. In order to assess CD4+ T cell responses, T cells are re-stimulated with the tissue-specific peptide of interest. After stimulation, production of cytokines by CD4+ T cells (e.g., IFNγ and TNFα) are assessed by intracellular staining. These cytokines, especially IFNγ, used to identify stimulated cells.

### Cell Expansion and Preparation

To prepare APCs, the following method is employed (a) obtain of autologous immune cells from the peripheral blood of the patient; enrich monocytes and dendritic cells in culture; load tissue-specific peptides and mature DCs.

### T Cell Induction (Protocol 1)

First induction: (a) Obtaining autologous T cells from an apheresis bag; (b) Depleting CD25+ cells and CD14+ cells, alternatively, depleting only CD25+ cells; (c) Washing the peptide loaded and mature DC cells, resuspending in the T cell culture media; (d) Incubating T cells with the matured DC.

Second induction: (a) Washing T cells, and resuspending in T cell media, and optionally evaluating a small aliquot from the cell culture to determine the cell growth, comparative growth and induction of T cell subtypes and antigen specificity and monitoring loss of cell population; (b) Incubating T cells with mature DC.

Third induction: (a) Washing T cells, and resuspending in T cell media, and optionally evaluating a small aliquot from the cell culture to determine the cell growth, comparative growth and induction of T cell subtypes and tissue-specific antigen specificity and monitoring loss of cell population; (b) Incubating T cells with mature DC.

To harvest peptide activated t cells and cryopreserve the T cells, the following method can be employed (a) Washing and resuspension of the final formulation comprising the activated T cells which are at an optimum cell number and proportion of cell types that constitutes the desired characteristics of the Drug Substance (DS). The release criteria testing include inter alia, Sterility, Endotoxin, Cell Phenotype, TNC Count, Viability, Cell Concentration, Potency; (b) Filling drug substance in suitable enclosed infusion bags; (c) Preservation until time of use.

### Methods of Functional Characterization of the CD4+ and CD8+Tissue-Specific Antigen-Specific T Cells.

T cell manufacturing processes were developed to raise memory and de novo CD4+ and CD8+ T cell responses to tissue-specific antigens through multiple rounds of ex-vivo T cell stimulation, generating a tissue-specific antigen-reactive T cell product for use in adoptive cell therapy. Detailed characterization of the stimulated T cell product can be used to test the many potential variables these processes utilize.

To probe T cell functionality and/or specificity, an assay was developed to simultaneously detect tissue-specific antigen-specific T cell responses and characterize their magnitude and function. This assay employs the following steps. First T cell-APC co-cultures were used to elicit reactivity in tissue-specific antigen-specific T cells. Optionally, sample multiplexing using fluorescent cell barcoding is employed. To identify tissue-specific antigen-specific CD8+ T cells and to examine T cell functionality, staining of peptide-MHC multimers and multiparameter intracellular and/or cell surface cell marker staining were probed simultaneously using FACS analysis. The results of this streamlined assay demonstrated its application to study T cell responses induced from a healthy donor. Tissue-specific antigen-specific T cell responses induced toward peptides are identified in a donor. The magnitude, specificity and functionality of the induced T cell responses are also compared. Briefly, different T cell samples are barcoded with different fluorescent dyes at different concentrations (see, e.g.. Example 19). Each sample receives a different concentration of fluorescent dye or combination of multiple dyes at different concentrations. Samples are resuspended in phosphate-buffered saline (PBS) and then fluorophores dissolved in DMSO (typically at 1:50 dilution) are added to a maximum final concentration of 5 μM. After labeling for 5 min at 37° C., excess fluorescent dye is quenched by the addition of protein-containing medium (e.g. RPMI medium containing 10% pooled human type AB serum). Uniquely barcoded T cell cultures are challenged with autologous APC pulsed with the tissue-specific antigen peptides as described above.

The differentially labeled samples are combined into one FACS tube or well, and pelleted again if the resulting volume is greater than 100 μL. The combined, barcoded sample (typically 100 μL) is stained with surface marker antibodies including fluorochrome conjugated peptide-MHC multimers. After fixation and permeabilization, the sample is additionally stained intracellularly with antibodies targeting TNF-α and IFN-γ.

The cell marker profile and MHC tetramer staining of the combined, barcoded T cell sample are then analyzed simultaneously by flow cytometry on flow cytometer. Unlike other methods that analyze cell marker profiles and MHC tetramer staining of a T cell sample separately, the simultaneous analysis of the cell marker profile and MHC tetramer staining of a T cell sample described in this example provides information about the percentage of T cells that are both tissue-specific antigen specific and that have increased cell marker staining. Other methods that analyze cell marker profiles and MHC tetramer staining of a T cell sample, separately determine the percentage of T cells of a sample that are tissue-specific antigen specific, and separately determine the percentage of T cells that have increased cell marker staining, only allowing correlation of these frequencies.

The simultaneous analysis of the cell marker profile and MHC tetramer staining of a T cell sample described in this example does not rely on correlation of the frequency of tissue-specific antigen specific T cells and the frequency of T cells that have increased cell marker staining: rather, it provides a frequency of T cells that are both tissue-specific antigen specific and that have increased cell marker staining. The simultaneous analysis of the cell marker profile and MHC tetramer staining of a T cell sample described in this example allows for determination on a single cell level, those cells that are both tissue-specific antigen specific and that have increased cell marker staining.

To evaluate the success of a given induction process, a recall response assay may be used followed by a multiplexed, multiparameter flow cytometry panel analysis. A sample taken from an induction culture is labeled with a unique two-color fluorescent cell barcode. The labeled cells are incubated on tissue-specific antigen-loaded DCs or unloaded DCs overnight to stimulate a functional response in the tissue-specific antigen-specific cells. The next day, uniquely labeled cells are combined prior to antibody and multimer staining.

### Exemplary Materials for T Cell Culture are Provided Below:

Materials: AIM V media (Invitrogen)Human FLT3L; preclinical CellGenix #1415-050 Stock 50 ng/μL TNFα; preclinical CellGenix #1406-050 Stock 10 ng/μL; IL-1β, preclinical CellGenix #1411-050 Stock 10 ng/μL; PGE1 or Alprostadil—Cayman from Czech republic Stock 0.5 μg/μL; R10 media-RPMI 1640 glutamax+10% Human serum+1% PenStrep: 20/80 Media-18% AIM V+72% RPMI 1640 glutamax+10% Human Serum+1% PenStrep; IL7 Stock 5 ng/μL: IL15 Stock 5 ng/μL; DC media (Cellgenix); CD14 microbeads, human, Miltenyi #130-050-201. Cytokines and/or growth factors. T cell media (AIM V+RPMI 1640 glutamax+serum+PenStrep), Peptide stocks—1 mM per peptide tissue-specific peptides).

### Example 11. Discovery Approach

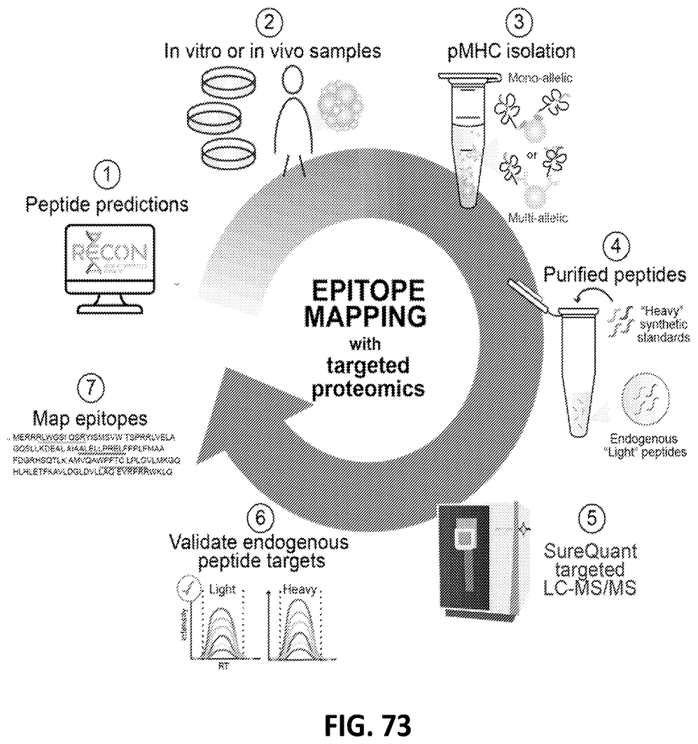

In this example, a discovery approach for MHC-epitope and cognate TCRs for effective T cell therapeutics is described (FIG. 73).

### MHC-I Peptide Enrichment

Frozen cell pellets endogenously expressing MHC molecules (untagged) or biotin acceptor peptide (BAP)-tagged MHC molecules were lysed by pipetting and end-over-end rotation for twenty minutes using lysis buffer [20 mM Tris-Cl pH 8, 100 mM NaCl, 6 mM MgCl2, 1.5% (v/v) Triton X-100, 60 mM octyl B-D-glucopyranoside, 0.2 mM of 2-Iodoacetamide, 1 mM EDTA pH 8, 1 mM PMSF, 1× complete EDTA-free protease inhibitor cocktail (Roche)] plus benzonase nuclease for twenty minutes. Tissue samples were homogenized in lysis buffer plus benzonase nuclease. All lysates were cleared by centrifugation. Samples with untagged MHC molecules were subsequently incubated with GammaBind Plus Sepharose Beads (GE Healthcare) pre-charged with a pan-HLA A/B/C antibody (clone W6/32) overnight at 4 C with end-over-end rotation. BAP-tagged samples were biotinylated with 0.56 μM biotin, 1 mM ATP, and 1 μM BirA biotin ligase for 10 minutes, and subsequently incubated with High-Capacity NeutrAvidin Agarose resin for 30 minutes at 4° C. with end-over-end rotation. Following enrichment, beads were washed 2× with wash buffer A [20 mM Tris-Cl pH 8, 100 mM, NaCl, 60 mM octyl B-D-glucopyranoside, 0.2 mM of 2-Iodoacetamide, 1 mM EDTA pH 8] and wash buffer B [10 mM Tris-Cl pH 8] using a positive pressure manifold. MHC molecules were eluted using 10% acetic acid and peptides were isolated using 10K molecular weight cut-off filtration following filter passivation with 1% bovine serum albumin (BSA). If required, samples were next reduced and alkylated using 5 mM Bond-Breaker TCEP solution at 60° C. for 30 min followed by 15 mM 2-Iodoacetamide for 30 min, protected from light. Samples were next acidified using 100% formic acid and desalted using 10 mg Sep-Pak tC18 μElution plates with peptide elutions at 15% acetonitrile and 50% acetonitrile, which were subsequently pooled. The volume of eluted peptides was reduced using vacuum centrifugation.

### Discovery Mass Spectrometry MHC-Peptide Analysis

For discovery approach and analyses (unbiased identification of presented MHC-peptides), peptides were resuspended in 3% acetonitrile, 5% formic acid and analyzed using liquid chromatography-mass spectrometry with a data dependent acquisition (DDA) methodology.

### Spectral Searching of Internal and Published DDA Datasets for Class I MHC Peptides

Raw mass spectra files generated in house or published datasets accessed using the PRoteomics IDEntifications (PRIDE) database repository or Mass Spectrometry Interactive Virtual Environment (MassIVE) database repository were searched using Spectrum Mill software package (version BI.07.04.210) against all UCSC Genome Browser genes (January 2018, Homo sapiens) and common contaminants. Searches included oxidated methionine as a variable modification in all searches, and carboxymethylation of cystine residues as a variable modification when sample processing included cystine reduction and alkylation steps. A minimum scored peak intensity (SPI) of 50% & PSM FDR estimate <1% was used to filter results. All sequences between 7 and 17 amino acids in length were considered.

### Targeted Mass Spectrometry MHC-Peptide Analysis

For targeted analyses, isolated MHC-I peptides were labeled using an isobaric labeling reagent from the tandem mass tag (TMT) 10-plex reagent set (Thermo Fisher). Dried peptides were resuspended in 50 mM HEPES buffer pH 8.5 and combined with 33.3 μg of TMT solubilized in 100% anhydrous acetonitrile. Peptides were incubated for 1 hour at room temperature after which the reaction was quenched with hydroxylamine. Peptides were subsequently dried by vacuum centrifugation, and resuspended in 3% acetonitrile, 5% formic acid. Prior to analysis, heavy isotope-labeled synthetic peptides corresponding to epitope targets of interest were labeled with Super Heavy TMT labeling reagent (Thermo Fisher) as—previously described. Dried, labeled synthetic peptides were resuspended in 3% acetonitrile, 5% formic acid and 100 fmol of each peptide was added to the isolated, TMT-10plex labeled enriched peptide mixture.

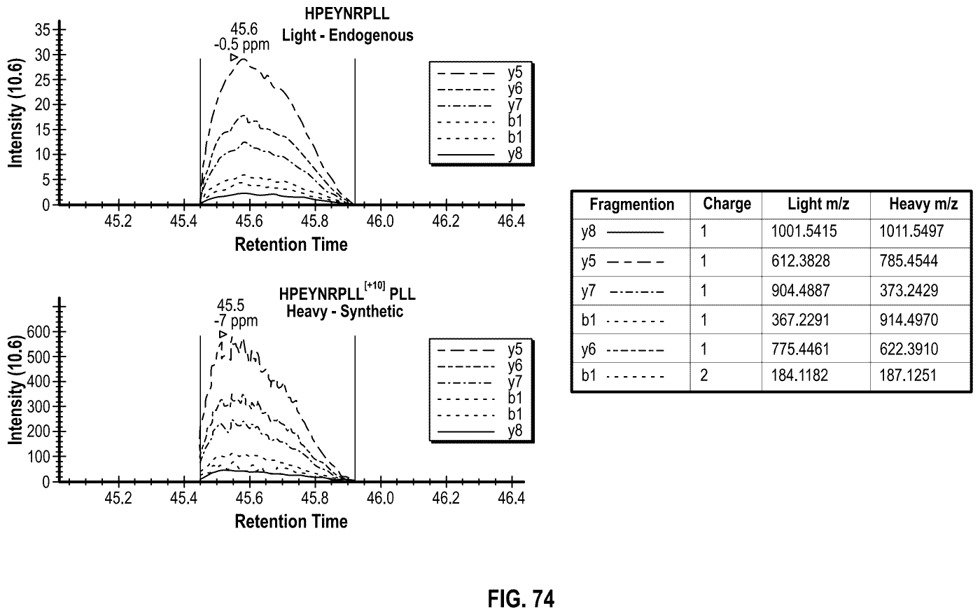

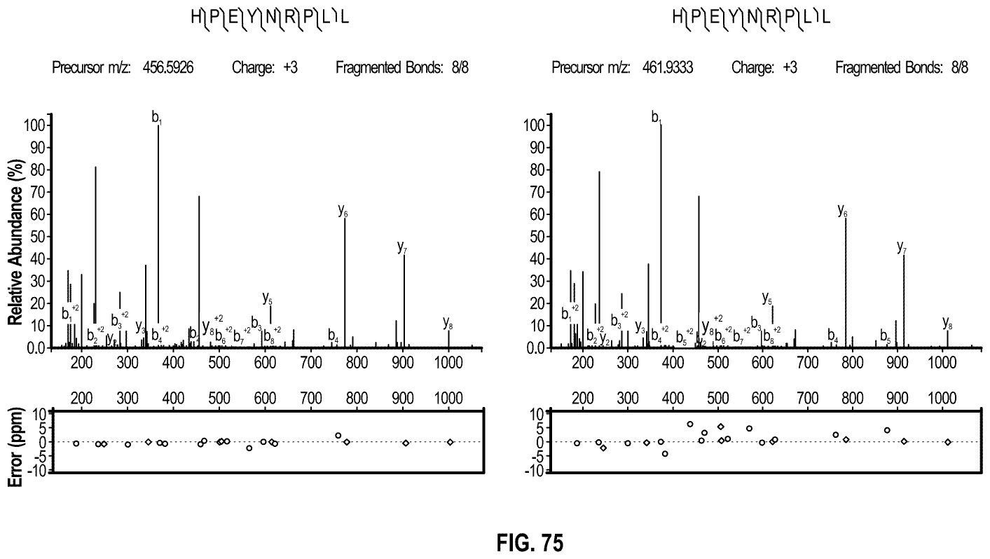

Peptides were analyzed using SureQuant targeted data acquisition strategy, where the heavy isotope labeled synthetic peptide serves as a trigger to guide the acquisition of spectrum corresponding to the light (unlabeled) endogenous MHC peptide using mass offset triggering and pseudo-spectral matching. All analyses were analyzed in Skyline, where the detection of an endogenous peptide was verified by matching retention times and spectral similarity between the heavy synthetic peptide and the light endogenous peptide (FIG. 74). Spectral similarity metrics include the dot product score and manual validation by comparing intensity distributions of 6 pre-selected product ions. Chromatographs of 6 characteristic fragment ions for the light (endogenous) and heavy isotope-labeled synthetic peptide sequence “HPEYNRPLL (SEQ ID NO: 8%9)” derived from KLK4 (HLA*B-07:02, where the endogenous peptide was identified in a human prostate specimen) are shown. Matched chromatography retention times and a high dot product similarity score (0.992, calculated using Skyline software) of peptide fragment ions provide validation that this epitope is processed and presented on the HLA-B*07:02 molecule. Two exemplary spectrums showing spectral validation of endogenous peptides using targeted proteomics are shown in FIG. 75. Spectrum for the light (endogenous) HPEYNRPLL (SEQ ID NO: 4143) epitope identified on a human prostate specimen (left) and the corresponding heavy isotope-labeled synthetic peptide (right) are shown. B and Y fragment ions are shown, and display high spectral similarity, confirming detection of the endogenous epitope. For each peptide, the top 200 more intense ions were plotted, and corresponding mass error of highlighted b and y ions are plotted below spectrum plots.

### NeoStim TCR Identification Protocol

In vitro T cell inductions were used to prime, enrich, and expand antigen specific T cells. Healthy human donor PMBCs were seeded into multiple wells of a GREX 24 well flask with FLT3-L in AIM-V media (Invitrogen). Inducing peptides, TNF-α, IL-1β, PGE1, and TL-7 were added into wells after 24 hours. After an overnight incubation, human serum was added to the wells to a final concentration of 5%. The culture media was increased to 7 mL 48 hours following the addition of human serum, the added media contained 5% human serum, IL-7, and IL-15. The IL-7 and IL-15 concentration was maintained throughout the culture by supplementing the cultures with the cytokines every 48-72 hours.

On Day 13 of culture, the inducing peptides are reintroduced to the cultures for 24 hours. The cultures are then harvested and wells with the same inducing peptides were combined to achieve a total cell number >100e6. These pooled samples were then enriched for CD137 using the Miltenyi CD137 GMP MACS kit and LS columns with a 70 um pre-separation filter.

Enriched cultures were then expanded in AIM-V media containing IL-2, IL-7, IL-15, human serum, anti-CD28 antibody, and in some cases, glucose, non-essential amino acids, and vitamins for 24 hours. In some cases, inducing peptides may have been added in an increasing peptide concentration for the three days following enrichment (days 15, 16, and 17 of the culture). On day 19 of the culture, the culture volume was increased to 6 mL via the addition of AIM-V media containing IL-2, IL-7, IL-15, human serum, glucose, non-essential amino acids, and vitamins.

The cultures were harvested on Day 26 following the start of the culture. Once harvested the cells were frozen in FBS supplemented with 10% DMSO or analyzed for multimer staining immediately after harvest. The frozen samples were moved to long term liquid nitrogen storage.

The cells were stained with CD14, CD16, CD19, CD8, and CD4 as linage markers and a suite of multimers loaded with the inducing peptides. Antigen specific cells were identified as CD14− CD16− CD19− CD4− CD8+ positive for the unique peptide fluorophores and negative for the other fluorophores

### Multimer Results

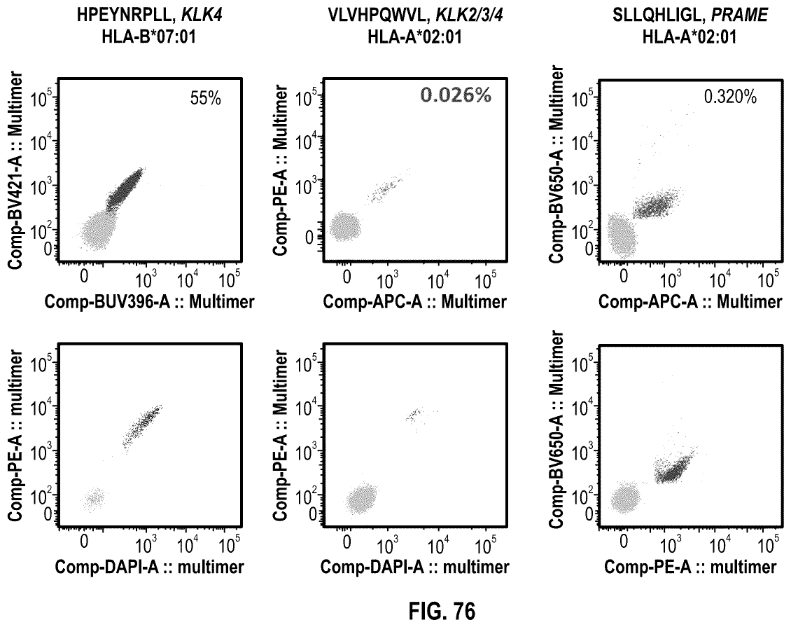

FIG. 76 depicts exemplary flow cytometry plots of peptide-MHC multimer staining of target epitopes after naive T cell inductions in healthy donors with the indicated HLA-I molecules. Multimer positive populations and the percentage of multimer positive cells is shown. The top panel displays positive sample identifications using a combinatorial multimer analysis. The bottom panel displays results from a confirmation combinatorial analysis performed on frozen samples following the initial identification from the top panel. Multimer positive cells from analyses in the bottom panel are sorted for downstream TCR identification.

### TCR Identification

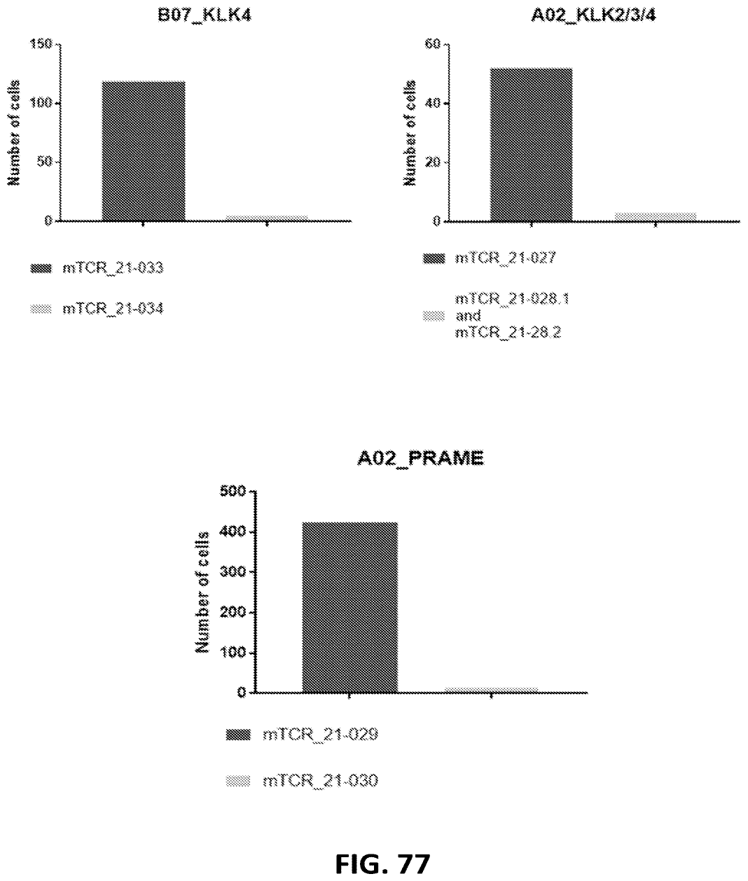

FIG. 77 depicts graphs showing exemplary TCR clonotypes identified from the 10× genomics pipeline. Each graph originated from a single sorted, multimer positive, population. The samples in this case all contained two unique TCR clonotypes, identified by a paired alpha and beta sequence. In the case where the 10× genomics pipeline identified a clonotype that contained multiple of either alpha or beta sequences, all possible combinations were synthesized for antigen specificity and avidity.

### Transfection and Lentivirus Production

Lentivirus encoding antigen-specific TCRs was prepared by the LV-MAX Lentiviral Production System supplied by Gibco using the protocol to produce Lentivirus in a 50 mL conical tube. Following the transient transfection, the lentivirus was tittered using Lenti-X GoStix from Takara and then concentrated 10 fold using Lenti-X Concentrator from Takara.

### CD8 Transduction of Jurkat Cells

2e6 CD8 Jurkat cells were plated in a 24 well plate in 1 mL RPMI supplemented with 10% FBS and 200 μL Lentiblast. Concentrated virus was added to the well, at GV ˜40,000 add 100 uL, adding at most 1 mL to each culture. The cells were spinfected at 2400 rpm, 32C, for 45 minutes and incubated overnight. On the following day the plates were spun and either the media was changed to fresh RPMI with no virus, or the spinfection was repeated for a total of 2 times.

The cells were cultured for a total of 7 days in the 24 well plate before they were expanded to a GREX 24 flask and put under puro selection. Following 48 hours of selection, the cells were used for downstream analyses.

### Jurkat TCR-pMHC Recognition Assay

The coculture is to be done at an effector to target ratio of 5:1. The target cell number can vary between 50,000 and 10,000 cells with an according number of effector cells to maintain the ratio. For adherent cells, the target cells are plated for between 2 hours and overnight before peptide is added. Peptides are serially diluted to a range between 10 μM and 0.1 nM final concentration and are added at least 1 hour prior to addition of Jurkat cells. Prior to addition to the coculture, Jurkat cells are washed and resuspended in RPMI supplemented with 10% FBS.

The cells were co-cultured overnight before harvest and staining for CD69 expression via flow using a CD8, CD3, and murine TCR constant antibodies as lineage markers for effector cells.

### TCR Avidity Measurements

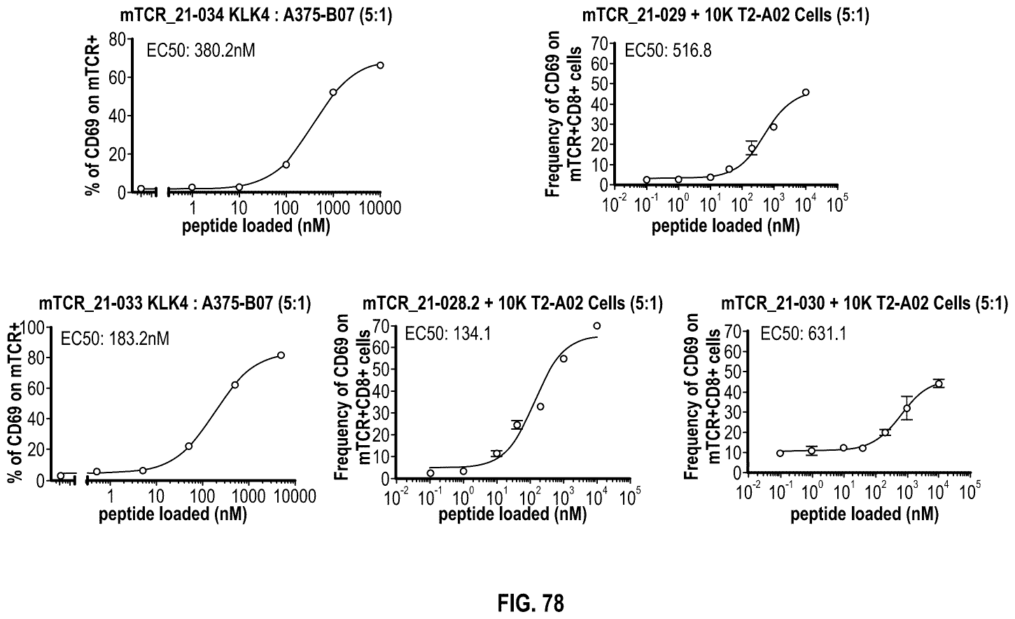

Target A375 cells or T2 cells were transduced to overexpress the allele of interest. A375 cells were plated at a density of 50 k per well and T2 cells were plated at a density of 10K per well, and were peptide pulsed for 1 hour at a final concentration between 10e3 and 10e-1 nM. Cells were co-cultured with Jurkat effector cells transduced to express the TCR of interest overnight at a 5:1 effector:target ratio before harvest. Cells were stained for CD69 expression using flow cytometry with CD8, CD3, and murine TCR constant antibodies as cell linear markers for effector cells. Data is reported as percentage of CD69 positive cells among TCR-expressing Jurkat cells. FIG. 78 depicts exemplary plots showing avidity of exemplary TCRs. The plots reflect the CD69 expression on transduced Jurkat cells (identified by the co-expression of murine TCR. CD8, and CD3) following an overnight coculture with a target cell line presenting the HLA and loaded with a variable amount of peptide. Of the seven TCRs tested, five of them show increased expression of CD69 in a peptide dependent manner. The concentration required to achieve a 50% activation (EC50) is calculated from these plots and the results are shown on the plot.

The following Table 5 shows exemplary results of TCR discovery using the protocols above.

TABLE 5
TCR discovery results

| | | | | Reactive T cell |
|---|---|---|---|---|
| | SEQ | | | population |
| Epitope | ID NO: | Allele | Gene | gencrated |
| SLSKILDTV | 826 | A02:01 | ANKRD30A | Y |
| LLSHGAVIEV | 831 | A02:01 | ANKRD30A | Y |
| SLQCVSLHL | 3456 | A02:01 | KLK2 | Y |
| VLVHPQWVL | 3757 | A02:01 | KLK2/3/4 | Y |
| SLFHPEDTGQV | 3775 | A02:01 | KLK3 | Y |
| HPEYNRPLL | 4143 | B07:02 | KLK4 | Y |
| KIWEELSVLEV | 8966 | A02:01 | MAGEA3 | Y |
| KVLEHVVRV | 4643 | A02:01 | MAGEA4 | Y |
| GLSNLTHVL | 8967 | A02:01 | PRAME | Y |
| SLLQHLIGL | 6095 | A02:01 | PRAME | Y |
| PYLGQMINL | 6120 | A24:02 | PRAME | Y |
| SPSVSQLSVL | 6139 | B07:02 | PRAME | Y |
| MPMQDIKMIL | 8968 | B07:02 | PRAME | Y |

A T cell population reactive to each of the above epitope:MHC complexes has been generated.

### Endogenous TCR Activity Assay

MDA-PCa-2b cells were plated at 50K/well in F12K media. The next day the cultures were treated with a cocktail of interferon alpha, beta, and gamma all at 1 U/uL final concentration. The next day the cells were washed with RPMI supplemented with 10% FBS and Glutamax. The cultures were then pulsed with peptide at a final concentration of 2 μM for 1 hour before the addition of effector cells.

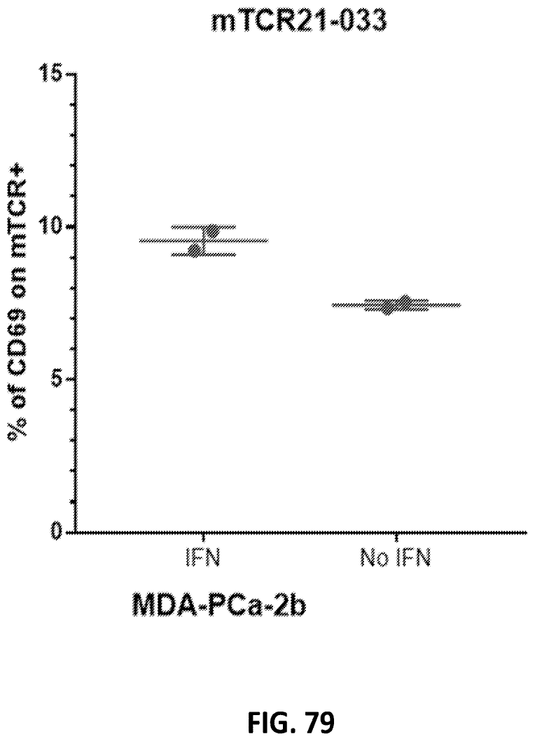

The cells were co-cultured overnight before harvest and staining for CD69 expression via flow using a CD8, CD3, and murine TCR constant antibodies as lineage markers for effector cells and HLA-B07 as a lineage marker for the target cells. FIG. 79 depicts exemplary plots showing endogenous activity of two different exemplary TCRs, avidity of exemplary TCRs, the plots here reflect the activation of two different TCR sequences (hereafter named mTCR21-033 and mTCR-034) following a coculture with the cell line MDA-PCa-2b which is endogenous for both HLA-B07 and KLK4. These plots are showing an increase in activation of mTCR21-033, but not mTCR21-034 following a 24-hour treatment with a cocktail of interferons (IFN). The IFN treatment increases the expression of surface HLA on cell lines, and the increased surface expression of the HLA can provide more expression of HLA-B07 bound to the KLK4 epitope.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-94. (canceled)

95. A method of treating a disease or condition in a human subject in need thereof, comprising:
administering a composition to the human subject, wherein the composition comprises:
(a) a tissue-specific antigen peptide comprising an epitope sequence of a protein, wherein the epitope sequence is expressed by a diseased cell of a target tissue of the human subject;
(b) a polynucleotide encoding the tissue-specific antigen peptide;
(c) one or more antigen presenting cells (APCs) presenting the epitope sequence;
(d) a T cell receptor (TCR) specific to a complex of (i) the epitope sequence and (ii) a protein encoded by an HLA allele of the human subject; or
(e) a population of immune cells from a biological sample comprising at least one antigen specific T cell comprising the TCR;
wherein the epitope sequence binds to or is predicted to bind to a protein encoded by an HLA allele expressed by the human subject, and
wherein the tissue-specific antigen epitope gene has an expression level in the diseased cell of a target tissue of the human subject that is at least 2 fold more than an expression level of the tissue-specific antigen epitope gene in cells of each non-target tissue of the subject, wherein the tissue-specific antigen is specific to a non-essential tissue, and
wherein the non-essential tissue is a tissue not required for survival of a human.

96. The method of claim 95, wherein human subject does not have the non-essential tissue or the function of the non-essential tissue is substituted.

97. The method of claim 95, wherein human subject has the non-essential tissue.

98. The method of claim 95, wherein the tissue-specific antigen peptide comprising an epitope sequence of a protein is expressed in the target tissue and not expressed in each tissue of a plurality of non-target tissues that are different than the target tissue.

99. The method of claim 95, wherein the diseased cell is a tumor cell of the target tissue.

100. The method of claim 95, wherein an mRNA expression level of a gene encoding the tissue-specific antigen peptide is at the most about 5 mRNA transcripts in a non-target essential tissue per one million total mRNA transcripts in each respective non-target tissue of the subject; and wherein an mRNA expression level of the gene encoding the tissue-specific antigen peptide is at least about 100 mRNA transcripts per one million total mRNA transcripts in the target tissue.

101. The method of claim 96, wherein
(i) the human subject is a female subject, and the tissue-specific antigen is specific to a non-essential tissue selected from the group consisting of: Bulbourethral gland, epididymis, penis, prostate, scrotum, seminal vesicle, testicle, and any combination thereof,
(ii) the human subject is a male subject, and the tissue-specific antigen is specific to a non-essential tissue selected from the group consisting of: Bartholin's gland, fallopian tube, ovary, Skene's gland, uterus, cervix, vagina, and any combination thereof,
(iii) the human subject is a Type I diabetes patient, and the tissue-specific antigen is specific to pancreas; or
(iv) the human subject has auto-immune thyroid condition, and the tissue-specific antigen is specific to thyroid.

102. The method of claim 96, wherein the human subject is a female subject, and the tissue-specific antigen is specific to prostate.

103. The method of claim 95, wherein the tissue-specific antigen peptide of (a) is an isolated, purified, and/or synthetic peptide; and wherein the tissue-specific antigen peptide further comprises an accessory sequence flanking the epitope sequence.

104. The method of claim 95, wherein the polynucleotide encoding the tissue-specific antigen peptide of (b) is an mRNA.

105. The method of claim 95, wherein the at least one antigen specific T cell comprises an exogenous sequence encoding the TCR.

106. The method of claim 95, wherein the tissue-specific antigen peptide comprising the epitope sequence of the polypeptide is encoded by the gene selected from the group consisting of ANKRD30A, COL10A1, CTCFL, PPIAL4G, POTEE, DLL3, MMP13, SSX1, DCAF4L2, MAGEA4, MAGEA11, MAGEC2, MAGEA12, PRAME, CLDN6, EPYC, KLK3, KLK2, KLK4, TGM4, POTEG, RLN1, POTEH, SLC45A2, TSPAN10, PAGE5, CSAG1, PRDM7, TG, TSHR, RSPH6A, SCXB, HIST1H4K, ALPPL2, PRM2, PRM1, TNP1, LELP1, HMGB4, AKAP4, CETN1, UBQLN3, ACTL7A, ACTL9, ACTRT2, PGK2, C2orf53, KIF2B, ADAD1, SPATA8, CCDC70, TPD52L3, ACTL7B, DMRTB1, SYCN, CELA2A, CELA2B, PNLIPRP1, CTRC, AMY2A, SERPINI2, RBPJL, AQP12A, IAPP, KIRREL2, G6PC2, AQP12B, CYP11BI, CYP11B2, STAR, CYP11A1, and MC2R; or
wherein the epitope sequence has from 70% to 100% sequence identity to a peptide sequence selected from the group consisting of SEQ ID NOs 1-8962.

107. The method of claim 95, wherein the epitope sequence has from 70% to 100% sequence identity to a peptide sequence selected from the group consisting of SEQ ID NOs: 3441-4274, 5285-6084, 6580-6845, and 8100-8434.

108. The method of claim 95, wherein the protein comprising the tissue-specific antigen peptide comprising the epitope sequence is KLK2, KLK3 or KLK4, and wherein the non-essential tissue is prostate.

109. The method of claim 95, wherein the protein comprising the tissue-specific antigen peptide comprising the epitope sequence is KLK2.

110. The method of claim 95, wherein the epitope sequence has from 70% to 100% sequence identity to a peptide sequence selected from the group consisting of SEQ ID NOs: 3441-3721, 3723, 3729, 3736, 3744, 3752, 3757, 3778, 3794, 3804, 3811, 3813, 3816, 3822, 3824, 3829, 3831, 3833, 3835, 3844, 3850, 3854, 3854, 3869, 3879, 3905, 3916, 3923, 3928, 3944, 3959, 3961, 3972, 3975, 3993, 3998, 3999, and 4001.

111. The method of claim 95, wherein the epitope sequence is selected from the group consisting of AYSEKVTEF (SEQ ID NO: 3534), GLWTGGKDTCGV (SEQ ID NO: 3468), QRVPVSHSF (SEQ ID NO: 3544), SLQCVSLHL (SEQ ID NO: 3456), VILLGRHSL (SEQ ID NO: 3891), RPRSLQCVSL (SEQ ID NO: 3578), SLQCVSLHL (SEQ ID NO: 3456), and YSEKVTEFML (SEQ ID NO: 3454).

112. The method of claim 95, wherein the TCR is specific to a complex of:
1) the epitope sequence is AYSEKVTEF (SEQ ID NO: 3534) and the human subject expresses a protein encoded by an HLA-C06:02 or HLA-A24:02 allele,
2) the epitope sequence is GLWTGGKDTCGV (SEQ ID NO: 3468) and the human subject expresses a protein encoded by an HLA-A02:01 allele,
3) the epitope sequence is QRVPVSHSF (SEQ ID NO: 3544) and the human subject expresses a protein encoded by an HLA-C*07:01, HLA-C*07:02 or HLA-A24:02 allele,
4) the epitope sequence is SLQCVSLHL (SEQ ID NO: 3456) and the human subject expresses a protein encoded by an HLA-A02:01 allele,
5) the epitope sequence is RPRSLQCVSL (SEQ ID NO: 3578) and the human subject expresses a protein encoded by an HLA-B07:02 allele,
6) the epitope sequence is SLQCVSLHL (SEQ ID NO: 3456) and the human subject expresses a protein encoded by an HLA-A02:01 allele, or
7) the epitope sequence is YSEKVTEFML (SEQ ID NO: 3454) and the human subject expresses a protein encoded by an HLA-A01:01 allele.

113. The method of claim 112, wherein the antigen specific T cell comprises the TCR of any one of 1)-7).

114. A method of preparing T cells comprising a T cell receptor (TCR) specific to a complex of (i) an epitope sequence of a tissue specific antigen peptide and (ii) a protein encoded by an HLA allele of a human subject, the method comprising:
(a) contacting a T cell ex vivo with an antigen peptide in complex with an HLA of an APC, wherein the T cell has reduced on no tolerance for the antigenic peptide,
(b) determining a sequence of a TCR of the T cell that recognizes the antigen peptide in complex with the HLA, wherein the T cell has reduced or no immune tolerance to a tissue of origin of the antigen peptide;
(c) (i) expanding the T cell in presence of the antigen peptide in complex with the HLA, or (ii) expressing a recombinant polynucleic acid encoding the sequence of the TCR in isolated T cells;
thereby preparing the T cells comprising the TCR; and
wherein the T cell is from a subject, wherein the tissue-specific antigen is specific to a non-essential tissue, and wherein the non-essential tissue is a tissue not required for survival of a human.

115. A composition comprising:
(a) a tissue-specific antigen peptide comprising an epitope sequence of a polypeptide encoded by a gene selected from the group consisting of KLK2, KLK3 and KLK4;
(b) a polynucleotide encoding the tissue-specific antigen peptide;
(c) one or more antigen presenting cells (APCs) presenting the epitope sequence;
(d) a T cell receptor (TCR) or an antibody, or a functional part thereof that is specific to a complex of (i) the epitope sequence and (ii) a protein encoded by an HLA allele; or
(e) a population of immune cells from a biological sample comprising at least one antigen specific T cell comprising the TCR.
